SEQUENCE LISTING (411 sequences)

SEQ ID NO: 1 - protein, 296 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKDYKDDDDKGSGATNFSLLKQAGDVEENPGPASAGSGEGRGSLLTCGDVEENPGPATGNSA

SEQ ID NO: 2 - protein, 701 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 3 - protein, 701 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAAIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 4 - protein, 549 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKDYKDDDDKNSAWRWLPCQRHATSATNFSLLKQAGDVEENPGPGGSEGRGSLLTCGDVEENPGPSGYPYDVPDYAHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKAS

SEQ ID NO: 5 - protein, 247 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKDYKDDDDKNSA

SEQ ID NO: 6 - protein, 247 aa, synthetic construct; identical to SEQ ID NO: 5

SEQ ID NO: 7 - protein, 249 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKDYKDDDDKNSAWR

SEQ ID NO: 8 - protein, 247 aa, synthetic construct; identical to SEQ ID NO: 5

SEQ ID NO: 9 - protein, 296 aa, synthetic construct; identical to SEQ ID NO: 1

SEQ ID NO: 10 - protein, 296 aa, synthetic construct; identical to SEQ ID NO: 1

SEQ ID NO: 11 - protein, 296 aa, synthetic construct; identical to SEQ ID NO: 1

SEQ ID NO: 12 - protein, 714 aa, synthetic construct
MSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRASTGSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 13 - protein, 613 aa, synthetic construct
MADAQTRRRERRAEKQAQWKAANTGSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 14 - protein, 609 aa, synthetic construct
MASGPRPRGTRGKGRRIRRTGSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 15 - protein, 234 aa, synthetic construct
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLYGMDELYK

SEQ ID NO: 16 - protein, 281 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKKLSHGFPPEVEEQDDGTLPMSCAQESGMDRHPAACASARINV

SEQ ID NO: 17 - protein, 792 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDEFVDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 18 - protein, 999 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDEFVDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSAAGGSGGSAAASSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 19 - protein, 797 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGSGDMRPEIWIAQELRRIGDEFNAYYARRTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 20 - protein, 1004 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGSGDMRPEIWIAQELRRIGDEFNAYYARRTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSAAGGSGGSAAASSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 21 - protein, 797 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGGSGGSGRPEIWMTQGLRRLGDEANAYYARRTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 22 - protein, 959 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGGSGGSGRPEIWMTQGLRRLGDEANAYYARRTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGTGGPGDELYRQSLEIISRYLREQATGAKDTKPMGRSGATSRKALETLRRVGDGVQRNHETAFQGMLRKLDIKNEDDVKSLSRVMIHVFSDGVTNWGRIVTLISFGAFVAKHLKTINQESCIEPLAESITDVLVRTKRDWLVKQRGWDGFVEFFHVEDLEGGGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 23 - protein, 999 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDELVDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSAAGGSGGSAAASSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 24 - protein, 959 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGGSGGSGRPEIWMTQGLRRLGDEINAYYARRTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGTGGPGDELYRQSLEIISRYLREQATGAKDTKPMGRSGATSRKALETLRRVGDGVQRNHETAFQGMLRKLDIKNEDDVKSLSRVMIHVFSDGVTNWGRIVTLISFGAFVAKHLKTINQESCIEPLAESITDVLVRTKRDWLVKQRGWDGFVEFFHVEDLEGGGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 25 - protein, 781 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASPSRLEEELRRRLTEPTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 26 - protein, 909 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASPSRLEEELRRRLTEPTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGGTAEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSGAGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 27 - protein, 784 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGPGRLEEELRRRLSPGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 28 - protein, 912 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGPGRLEEELRRRLSPGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGGTAEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSGAGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 29 - protein, 784 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGPGRLEQEIRARLSPGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 30 - protein, 912 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGPGRLEQEIRARLSPGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGGTAEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSGAGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 31 - protein, 330 aa, synthetic construct
MVAGHASGSPAFGTASHSNCEHEEIHLAGSIQPHGALLVVSEHDHRVIQASANAAEFLNLGSVLGVPLAEIDGDLLIKILPHLDPTAEGMPVAVRCRIGNPSTEYCGLMHRPPEGGLIIELERAGPSIDLSGTLAPALERIRTAGSLRALCDDTVLLFQQCTGYDRVMVYRFDEQGHGLVFSECHVPGLESYFGNRYPSSTVPQMARQLYVRQRVRVLVDVTYQPVPLEPRLSPLTGRDLDMSGCFLRSMSPCHLQFLKDMGVRATLAVSLVVGGKLWGLVVCHHYLPRFIRFELRAICKRLAERIATRITALESLESRLEEELRRRLTE

SEQ ID NO: 32 - protein, 784 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGGSGAHIVMVDAYKPTKGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 33 - protein, 904 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGGSGAHIVMVDAYKPTKGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGTSGGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 34 - protein, 947 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGGSGAHIVMVDAYKPTKGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNTSATNFSLLKQAGDVEENPGPGGSEGRGSLLTCGDVEENPGPGTSGGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIG

SEQ ID NO: 35 - protein, 914 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGGSGAHIVMVDAYKPTKGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGGTENLYFQSGTSGGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 36 - protein, 235 aa, synthetic construct
MGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQL

SEQ ID NO: 37 - protein, 1238 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDELVDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSGGVIPDYFKQSFPEGYSWERSMTYEDGGICIATNDITMEGDSFINKIHFKGTNFPPNGPVMQKRTVGWEASTEKMYERDGVLKGDVKMKLLLKGGGHYRCDYRTTYKVKQKPVKLPDYHFVDHRIEILSHDKDYNKVKLYEHAVARNSTDSMDELYKGGSGGMVSKGEETITSVIKPDMKNKLRMEGNVNGHAFVIEGEGSGKPFEGIQTIDLEVKEGAPLPFAYDILTTAFHYGNRVFTKYPRSGSGSSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 38 - DNA, 1895 bp, synthetic construct
  source 1..1895: mol_type = other DNA
aggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcgaaacatgaggatcacccatgtactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctcatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggta

SEQ ID NO: 39 - DNA, 2328 bp, synthetic construct
  source 1..2328: mol_type = other DNA
gatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgc

SEQ ID NO: 40 - DNA, 2392 bp, synthetic construct
  source 1..2392: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgccataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 41 - DNA, 1870 bp, synthetic construct
  source 1..1870: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaagaattccgcgtggcgctggcttccttgccagcgccacgcgactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctcatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttcta

SEQ ID NO: 42 - DNA, 1870 bp, synthetic construct
  source 1..1870: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaagaattccgcgtagcgctggcttccttgccagcgccacgcgactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctcatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttcta

SEQ ID NO: 43 - DNA, 1870 bp, synthetic construct
  source 1..1870: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaagaattccgcgtagcgctagcttccttgccagcgccacgcgactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctcatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttcta

SEQ ID NO: 44 - DNA, 1888 bp, synthetic construct
  source 1..1888: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaagaattccgcgtagcgctggctttgccagcgccacgcgaaacatgaggatcacccatgtactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctcatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttcta

SEQ ID NO: 45 - DNA, 1888 bp, synthetic construct
  source 1..1888: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaagaattccgcgtagcgctagctttgccagcgccacgcgaaacatgaggatcacccatgtactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctcatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttcta

SEQ ID NO: 46 - DNA, 2044 bp, synthetic construct
  source 1..2044: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcgaaggagcagacgatatggcgtcgctccaatactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctcatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttcta

SEQ ID NO: 47 - DNA, 2038 bp, synthetic construct
  source 1..2038: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcggtaagggccctgaagaagggcccaactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctcatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttcta

SEQ ID NO: 48 - DNA, 2047 bp, synthetic construct
  source 1..2047: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcggtaggctcgtctgagctcattagctccgagccaactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctcatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttcta

SEQ ID NO: 49 - DNA, 2432 bp, synthetic construct
  source 1..2432: mol_type = other DNA
taatacgactcactatagggagacccaagctggctagaggatcgaacccggtaccgccaccatgtccaagacaatcgtgcttagcgtgggagaagcaactcgaaccctcacagaaatccaatcaacagccgaccgacagatttttgaagagaaagtgggtcccttggttggccggctcaggctcacagccagcttgagacagaacggagccaagactgcctatcgagtgaatctcaagctggaccaggccgatgtagtggacagtggtttgcccaaggttagatacacgcaagtgtggtcacacgatgtcactattgtggcaaactccaccgaggcaagcaggaagtccctgtatgacctgaccaaatctttggtggcgacgagccaagtagaggatctggtggtaaacctggtgcccctgggaagagcctcaaccggttctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 50 - DNA, 2129 bp, synthetic construct
  source 1..2129: mol_type = other DNA
taatacgactcactatagggagacccaagctggctagaggatcgaacccggtaccgccaccatggccgacgcacaaacacgacgacgtgagcgtcgcgctgagaaacaagctcaatggaaagctgcaaacaccggttctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 51 - DNA, 2117 bp, synthetic construct
  source 1..2117: mol_type = other DNA
taatacgactcactatagggagacccaagctggctagaggatcgaacccggtaccgccaccatggcctctggtcctcgtccccgtggtactcgtggtaaaggtcgccgtattcgtcgcaccggttctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 52 - DNA, 2835 bp, synthetic construct
  source 1..2835: mol_type = other DNA
cggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagactgcagcctcaggagatctgggcccctacttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcaccatggtggcgaattctccaggcgatctgacggttcactaaacgagctctgcttatataggcctcccaccgtacacgccacctcgacatactcgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagggatcctctagtcagctgacgcgtgctagcgatatcggcgcgccagcatttaaatctgtacagaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgcccggatcaacgtgtagcgctagctttgccagcgccacacgaaacatgaggatcacccatgtcggccgcactcctcaggtgcaggctgcctatcagaaggtggtggctggtgtggccaatgccctggctcacaaataccactgagatctttttccctctgccaaaaattatggggacatcatgaagccccttgggcatctgacttctggctaataaaggaaatttattttcattgcaatagtgtgttggaattttttgtgtctctcactcggaaggacatatgggagggcaaatcatttaaaacatcagaatgagt

SEQ ID NO: 53 - DNA, 2482 bp, synthetic construct
  source 1..2482: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcgggtcgggcaccggtgctccacccaatctctgggcagcgcagcgctacggccgtgagctcagaaggatgtccgatgagttcgtcgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 54 - DNA, 3103 bp, synthetic construct
  source 1..3103: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcgggtcgggcaccggtgctccacccaatctctgggcagcgcagcgctacggccgtgagctcagaaggatgtccgatgagttcgtcgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcgcggccggaggtagcggcggaagcgcggccgcttcaagtaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 55 - DNA, 2497 bp, synthetic construct
  source 1..2497: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcgggtcgggctctggagacatgcggccagagatttggatcgcacaggaactgaggcgcattggcgatgagttcaatgcatactatgcccgaagaaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 56 - DNA, 3118 bp, synthetic construct
  source 1..3118: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcgggtcgggctctggagacatgcggccagagatttggatcgcacaggaactgaggcgcattggcgatgagttcaatgcatactatgcccgaagaaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcgcggccggaggtagcggcggaagcgcggccgcttcaagtaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 57 - DNA, 2497 bp, synthetic construct
  source 1..2497: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggaggtagcggcggatctgggcgaccagaaatctggatgacacaaggtttacgcagactcggagatgaggcaaatgcttactatgctagacggaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 58 - DNA, 2983 bp, synthetic construct
  source 1..2983: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggaggtagcggcggatctgggcgaccagaaatctggatgacacaaggtttacgcagactcggagatgaggcaaatgcttactatgctagacggaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggtaccggcggtccaggggacgagttgtaccggcagtcgctggagattatctctcgctaccttcgggagcaggccaccggagccaaggacacaaagccaatgggcaggtctggggccaccagcaggaaggcgctggaaaccttacgacgggttggggatggcgtgcagcgcaaccacgagactgccttccaaggcatgcttcggaaactggacatcaaaaacgaagatgatgtgaaatcgttgtctagggtgatgatccatgttttcagcgacggcgtaacaaactggggcaggattgtgactctcatttcttttggtgcctttgtggctaaacacttgaaaaccataaaccaagaaagctgcatcgaaccattagcagaaagtatcacagacgttctcgtaaggacaaaacgggactggctagttaaacaaagaggctgggatgggtttgtggagttcttccatgtagaggacctagaaggtggcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 59 - DNA, 3103 bp, synthetic construct
  source 1..3103: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcgggtcgggcaccggtgctccacccaatctctgggcagcgcagcgctacggccgtgagctcagaaggatgtccgatgagctggtcgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcgcggccggaggtagcggcggaagcgcggccgcttcaagtaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 60 - DNA, 2983 bp, synthetic construct
  source 1..2983: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggaggtagcggcggatctgggcgaccagaaatctggatgacacaaggtttacgcagactcggagatgagataaatgcttactatgctagacggaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggtaccggcggtccaggggacgagttgtaccggcagtcgctggagattatctctcgctaccttcgggagcaggccaccggagccaaggacacaaagccaatgggcaggtctggggccaccagcaggaaggcgctggaaaccttacgacgggttggggatggcgtgcagcgcaaccacgagactgccttccaaggcatgcttcggaaactggacatcaaaaacgaagatgatgtgaaatcgttgtctagggtgatgatccatgttttcagcgacggcgtaacaaactggggcaggattgtgactctcatttcttttggtgcctttgtggctaaacacttgaaaaccataaaccaagaaagctgcatcgaaccattagcagaaagtatcacagacgttctcgtaaggacaaaacgggactggctagttaaacaaagaggctgggatgggtttgtggagttcttccatgtagaggacctagaaggtggcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 61 - DNA, 2449 bp, synthetic construct
  source 1..2449: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcccatcccgcctggaggaagaacttcggaggagacttactgagcctaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 62 - DNA, 2833 bp, synthetic construct
  source 1..2833: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcccatcccgcctggaggaagaacttcggaggagacttactgagcctaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggaggtaccgccgaagttcaattacaggaatcgggtggaggtctggtacaacctgggggctctcttcgcctgagttgcactgccagtggagttacgatttctgcacttaatgctatggcgatgggttggtatcgtcaggccccaggggaacgtcgcgtcatggtcgctgccgtttccgaacgtggcaatgctatgtaccgcgagtctgttcagggccgcttcacggttacccgcgattttacaaataaaatggtatcgttgcaaatggacaacttaaagccagaggacactgctgtgtactactgtcacgtccttgaagatcgtgtggattcctttcatgattattgggggcaggggactcaggtcactgtatcctcaggagctggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 63 - DNA, 2458 bp, synthetic construct
  source 1..2458: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggatcaggtccaggacgcctggaggaagaacttcggaggagactttctcctggaaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 64 - DNA, 2842 bp, synthetic construct
  source 1..2842: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggatcaggtccaggacgcctggaggaagaacttcggaggagactttctcctggaaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggaggtaccgccgaagttcaattacaggaatcgggtggaggtctggtacaacctgggggctctcttcgcctgagttgcactgccagtggagttacgatttctgcacttaatgctatggcgatgggttggtatcgtcaggccccaggggaacgtcgcgtcatggtcgctgccgtttccgaacgtggcaatgctatgtaccgcgagtctgttcagggccgcttcacggttacccgcgattttacaaataaaatggtatcgttgcaaatggacaacttaaagccagaggacactgctgtgtactactgtcacgtccttgaagatcgtgtggattcctttcatgattattgggggcaggggactcaggtcactgtatcctcaggagctggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 65 - DNA, 2458 bp, synthetic construct
  source 1..2458: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggatcaggtccaggccgcctggagcaggaaattcgggcaagactttctcctggaaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 66 - DNA, 2842 bp, synthetic construct
  source 1..2842: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggatcaggtccaggccgcctggagcaggaaattcgggcaagactttctcctggaaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggaggtaccgccgaagttcaattacaggaatcgggtggaggtctggtacaacctgggggctctcttcgcctgagttgcactgccagtggagttacgatttctgcacttaatgctatggcgatgggttggtatcgtcaggccccaggggaacgtcgcgtcatggtcgctgccgtttccgaacgtggcaatgctatgtaccgcgagtctgttcagggccgcttcacggttacccgcgattttacaaataaaatggtatcgttgcaaatggacaacttaaagccagaggacactgctgtgtactactgtcacgtccttgaagatcgtgtggattcctttcatgattattgggggcaggggactcaggtcactgtatcctcaggagctggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 67 - DNA, 1118 bp, synthetic construct
  source 1..1118: mol_type = other DNA
taatacgactcactatagggagacccaagctggctagcgtttaaacttaagcttatggtagcaggtcatgcctctggcagccccgcattcgggaccgcctctcattcgaattgcgaacatgaagagatccacctcgccggctcgatccagccgcatggcgcgcttctggtcgtcagcgaacatgatcatcgcgtcatccaggccagcgccaacgccgcggaatttctgaatctcggaagcgtactcggcgttccgctcgccgagatcgacggcgatctgttgatcaagatcctgccgcatctcgatcccaccgccgaaggcatgccggtcgcggtgcgctgccggatcggcaatccctctacggagtactgcggtctgatgcatcggcctccggaaggcgggctgatcatcgaactcgaacgtgccggcccgtcgatcgatctgtcaggcacgctggcgccggcgctggagcggatccgcacggcgggttcactgcgcgcgctgtgcgatgacaccgtgctgctgtttcagcagtgcaccggctacgaccgggtgatggtgtatcgtttcgatgagcaaggccacggcctggtattctccgagtgccatgtgcctgggctcgaatcctatttcggcaaccgctatccgtcgtcgactgtcccgcagatggcgcggcagctgtacgtgcggcagcgcgtccgcgtgctggtcgacgtcacctatcagccggtgccgctggagccgcggctgtcgccgctgaccgggcgcgatctcgacatgtcgggctgcttcctgcgctcgatgtcgccgtgccatctgcagttcctgaaggacatgggcgtgcgcgccaccctggcggtgtcgctggtggtcggcggcaagctgtggggcctggttgtctgtcaccattatctgccgcgcttcatccgtttcgagctgcgggcgatctgcaaacggctcgccgaaaggatcgcgacgcggatcaccgcgcttgagagcctcgagtcccgcctggaggaagaacttcggaggagacttactgagtaatctagagggccctattctatagtgtcacctaaatgctagagctcgctgatcagcctcgactgtgccttcta

SEQ ID NO: 68 - DNA, 2458 bp, synthetic construct
  source 1..2458: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggaggtagcggagcccacatcgtgatggtggacgcctacaagccgacgaagggaaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 69 - DNA, 2818 bp, synthetic construct
  source 1..2818: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggaggtagcggagcccacatcgtgatggtggacgcctacaagccgacgaagggaaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggtaccagcggaggcgccatggttgataccttatcaggtttatcaagtgagcaaggtcagtccggtgatatgacaattgaagaagatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 70 - DNA, 2947 bp, synthetic construct
  source 1..2947: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggaggtagcggagcccacatcgtgatggtggacgcctacaagccgacgaagggaaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatactagtgccacaaacttctctctgctaaagcaagcaggtgatgttgaagaaaacccagggcctggagggtccgagggcaggggaagtctcctaacatgcggggacgtggaggaaaatcccggaccaggtaccagcggaggcgccatggttgataccttatcaggtttatcaagtgagcaaggtcagtccggtgatatgacaattgaagaagatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattggataagggcccgtttaaacccgctgatcagcctcgactgtgccttcta

SEQ ID NO: 71 - DNA, 2848 bp, synthetic construct
  source 1..2848: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggaggtagcggagcccacatcgtgatggtggacgcctacaagccgacgaagggaaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggaggtacggagaatttgtattttcagagcggtaccagcggaggcgccatggttgataccttatcaggtttatcaagtgagcaaggtcagtccggtgatatgacaattgaagaagatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 72 - DNA, 890 bp, synthetic construct
  source 1..890: mol_type = other DNA
taatacgactcactatagggagacccaagctggctagttaagcttgccaccatgggcgagagccttttcaagggcccgagggactacaacccgatctccagcaccatctgtcacctgaccaacgagagcgacggtcacaccactagtctgtacggcatcggcttcggccccttcatcatcaccaacaagcatctgttcaggaggaataacggcacactgctggtgcaaagcctgcacggcgtgttcaaagtgaagaacacaaccaccctgcaacagcacctgatcgacggcagggacatgattatcatcaggatgcccaaggacttccccccctttccccagaaactgaagttcagggagccacaaagggaggagcgaatctgcctggtgaccaccaacttccagaccaagtccatgagcagcatggtctctgataccagctgcaccttccccagcagcgacggcatcttctggaagcactggattcagacgaaggatggccaatgcggcagcccattggtgagcactagggacggcttcatcgtgggcatccacagcgccagcaattttaccaataccaacaactacttcacgagcgtgccgaaaaacttcatggagctgttgaccaatcaagaggcgcagcagtgggtgagcggctggaggctgaacgccgacagcgttctttggggcggacataaggtgttcatggtcaagcccgaggaacccttccagcccgttaaggaagccactcagctttgataactcgagtctagagggcccgcggttcgaacaaaaactcatctcagaagaggatctgaatatgcataccggtcatcatcaccatcaccattgagtttaaacccgctgatcagcctcgactgtgccttcta

SEQ ID NO: 73 - DNA, 3820 bp, synthetic construct
  source 1..3820: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcgggtcgggcaccggtgctccacccaatctctgggcagcgcagcgctacggccgtgagctcagaaggatgtccgatgagctggtcgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggaagtggtggtgtgatccctgactacttcaagcagagcttccccgagggctacagctgggagcgcagcatgacctacgaggacggcggcatctgcatcgccaccaacgacatcacaatggagggggacagcttcatcaacaagatccacttcaagggcacgaacttcccccccaacggccccgtgatgcagaagaggaccgtgggctgggaggccagcaccgagaagatgtacgagcgcgacggcgtgctgaagggcgacgtgaagatgaagctgctgctgaagggcggcggccactatcgctgcgactaccgcaccacctacaaggtcaagcagaagcccgtaaagctgcccgactaccacttcgtggaccaccgcatcgagatcctgagccacgacaaggactacaacaaggtgaagctgtacgagcacgccgtggcccgcaactccaccgacagcatggacgagctgtacaagggtggcagcggtggcatggtgagcaagggcgaggagaccattacaagcgtgatcaagcctgacatgaagaacaagctgcgcatggagggcaacgtgaacggccacgccttcgtgatcgagggcgagggcagcggcaagcccttcgagggcatccagacgattgatttggaggtgaaggagggcgccccgctgcccttcgcctacgacatcctgaccaccgccttccactacggcaaccgcgtgttcaccaagtacccacggtcgggaagtggctcaagtaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 74 - protein, 393 aa, synthetic construct
PLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGQGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 75 - protein, 385 aa, synthetic construct
RTPMPQEFADSISQLVTQKFREVTTDLTPMHARHKALAGIVMTKGLDARQAQVVALSSGTKCISGEHLSDQGLVVNDCHAEVVARRAFLHFLYTQLELHLSKRREDSERSIFVRLKEGGYRLRENILFHLYVSTSPCGDARLHSPYEITTDLHSSKHLVRKFRGHLRTKIESGQGTVPVRGPSAVQTWDGVLLGEQLITMSCTDKIARWNVLGLQGALLSHFVEPVYLQSIVVGSLHHTGHLARVMSHRMEGVGQLPASYRHNRPLLSGVSDTEARQPGKSPPFSMNWVVGSADLEIINATTGRRSCGGPSRLCKHVLSARWARLYGRLSTRTPSPGDTPSMYCEAKLGAHTYQSVKQQLFKAFQKAGLGTWVRKPPEQQQFLLT

SEQ ID NO: 76 - protein, 379 aa, synthetic construct
HYEGRHIQYAKISQIVKERFNQLISNRSEYLKYSSSLAAFIIERAGQHEVVAIGTGEYNYSQDIKPDGRVLHDTHAVVTARRSLLRYFYRQLLLFYSKNPAMMEKSIFCTEPTSNLLTLKQNINICLYMNQLPKGSAQIKSQLRLNPHSISAFEANEELCLHVAVEGKIYLTVYCPKDGVNRISSMSSSDKLTRWEVLGVQGALLSHFIQPVYISSILIGDGNCSDTRGLEIAIKQRVDDALTSKLPMFYLVNRPHISLVPSAYPLQMNLEYKFLSLNWAQGDVSLEIVDGLSGKITESSPFKSGMSMASRLCKAAMLSRFNLLAKEAKKELLEAGTYHAAKCMSASYQEAKCKLKSYLQQHGYGSWIVKSPCIEQFNM

SEQ ID NO: 77 - protein, 383 aa, synthetic construct
SVENILTHEQRCAALVSAGFDLLLDERSPYWACKGTVAGVILEREIPRARGHVKEIYKLVALGTGSSCCAGWLEFSGQQLHDCHGLVIARRALLRFLFRQLLLATQGGPKGKEQSVLAPQPGPGPPFTLKPRVFLHLYISNTPKGAARDIYLPPTSEGGLPHSPPMRLQAHVLGQLKPVCYVAPSLCDTHVGCLSASDKLARWAVLGLGGALLAHLVSPLYSTSLILADSCHDPPTLSRAIHTRPCLDSVLGPCLPPPYVRTALHLFAGPPVAPSEPTPDTCRGLSLNWSLGDPGIEVVDVATGRVKANAALGPPSRLCKASFLRAFHQAARAVGKPYLLALKTYEAAKAGPYQEARRQLSLLLDQQGLGAWPSKPLVGKFRN

SEQ ID NO: 78 - protein, 502 aa, synthetic construct
MWTADEIAQLCYEHYGIRLPKKGKPEPNHEWTLLAAVVKIQSPADKACDTPDKPVQVTKEVVSMGTGTKCIGQSKMRKNGDILNDSHAEVIARRSFQRYLLHQLQLAATLKEDSIFVPGTQKGVWKLRRDLIFVFFSSHTPCGDASIIPMLEFEDQPCCPVFRNWAHNSSVEASSNLEAPGNERKCEDPDSPVTKKMRLEPGTAAREVTNGAAHHQSFGKQKSGPISPGIHSCDLTVEGLATVTRIAPGSAKVIDVYRTGAKCVPGEAGDSGKPGAAFHQVGLLRVKPGRGDRTRSMSCSDKMARWNVLGCQGALLMHLLEEPIYLSAVVIGKCPYSQEAMQRALIGRCQNVSALPKGFGVQELKILQSDLLFEQSRSAVQAKRADSPGRLVPCGAAISWSAVPEQPLDVTANGFPQGTTKKTIGSLQARSQISKVELFRSFQKLLSRIARDKWPHSLRVQKLDTYQEYKEAASSYQEAWSTLRKQVFGSWIRNPPDYHQFK

SEQ ID NO: 79 - protein, 1226 aa, Homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLRSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 80 - protein, 701 aa, Homo sapiens
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 81 - protein, 739 aa, Homo sapiens
MASVLGSGRGSGGLSSQLKCKSKRRRRRRSKRKDKVSILSTFLAPFKHLSPGITNTEDDDTLSTSSAEVKENRNVGNLAARPPPSGDRARGGAPGAKRKRPLEEGNGGHLCKLQLVWKKLSWSVAPKNALVQLHELRPGLQYRTVSQTGPVHAPVFAVAVEVNGLTFEGTGPTKKKAKMRAAELALRSFVQFPNACQAHLAMGGGPGPGTDFTSDQADFPDTLFQEFEPPAPRPGLAGGRPGDAALLSAAYGRRRLLCRALDLVGPTPATPAAPGERNPVVLLNRLRAGLRYVCLAEPAERRARSFVMAVSVDGRTFEGSGRSKKLARGQAAQAALQELFDIQMPGHAPGRARRTPMPQEFADSISQLVTQKFREVTTDLTPMHARHKALAGIVMTKGLDARQAQVVALSSGTKCISGEHLSDQGLVVNDCHAEVVARRAFLHFLYTQLELHLSKRREDSERSIFVRLKEGGYRLRENILFHLYVSTSPCGDARLHSPYEITTDLHSSKHLVRKFRGHLRTKIESGEGTVPVRGPSAVQTWDGVLLGEQLITMSCTDKIARWNVLGLQGALLSHFVEPVYLQSIVVGSLHHTGHLARVMSHRMEGVGQLPASYRHNRPLLSGVSDAEARQPGKSPPFSMNWVVGSADLEIINATTGRRSCGGPSRLCKHVLSARWARLYGRLSTRTPSPGDTPSMYCEAKLGAHTYQSVKQQLFKAFQKAGLGTWVRKPPEQQQFLLTL

SEQ ID NO: 82 - protein, 576 aa, Homo sapiens
MASNNHWFQSSQVPSFAQMLKKNLPVQPATKTITTPTGWSSESYGLSKMASKVTQVTGNFPEPLLSKNLSSISNPVLPPKKIPKEFIMKYKRGEINPVSALHQFAQMQRVQLDLKETVTTGNVMGPYFAFCAVVDGIQYKTGLGQNKKESRSNAAKLALDELLQLDEPEPRILETSGPPPFPAEPVVLSELAYVSKVHYEGRHIQYAKISQIVKERFNQLISNRSEYLKYSSSLAAFIIERAGQHEVVAIGTGEYNYSQDIKPDGRVLHDTHAVVTARRSLLRYFYRQLLLFYSKNPAMMEKSIFCTEPTSNLLTLKQNINICLYMNQLPKGSAQIKSQLRLNPHSISAFEANEELCLHVAVEGKIYLTVYCPKDGVNRISSMSSSDKLTRWEVLGVQGALLSHFIQPVYISSILIGDGNCSDTRGLEIAIKQRVDDALTSKLPMFYLVNRPHISLVPSAYPLQMNLEYKFLSLNWAQGDVSLEIVDGLSGKITESSPFKSGMSMASRLCKAAMLSRFNLLAKEAKKELLEAGTYHAAKCMSASYQEAKCKLKSYLQQHGYGSWIVKSPCIEQFNM

SEQ ID NO: 83 - protein, 583 aa, Homo sapiens
MASASQGADDDGSRRKPRLAASLQISPQPRPWRPLPAQAQSAWGPAPAPATYRAEGGWPQVSVLRDSGPGAGAGVGELGAARAWENLGEQMGKAPRVPVPPAGLSLPLKDPPASQAVSLLTEYAASLGIFLLFREDQPPGPCFPFSVSAELDGVVCPAGTANSKTEAKQQAALSALCYIRSQLENPESPQTSSRPPLAPLSVENILTHEQRCAALVSAGFDLLLDERSPYWACKGTVAGVILEREIPRARGHVKEIYKLVALGTGSSCCAGWLEFSGQQLHDCHGLVIARRALLRFLFRQLLLATQGGPKGKEQSVLAPQPGPGPPFTLKPRVFLHLYISNTPKGAARDIYLPPTSEGGLPHSPPMRLQAHVLGQLKPVCYVAPSLCDTHVGCLSASDKLARWAVLGLGGALLAHLVSPLYSTSLILADSCHDPPTLSRAIHTRPCLDSVLGPCLPPPYVRTALHLFAGPPVAPSEPTPDTCRGLSLNWSLGDPGIEVVDVATGRVKANAALGPPSRLCKASFLRAFHQAARAVGKPYLLALKTYEAAKAGPYQEARRQLSLLLDQQGLGAWPSKPLVGKFRN

SEQ ID NO: 84 - protein, 502 aa, Homo sapiens; identical to SEQ ID NO: 78

SEQ ID NO: 85 - protein, 13 aa, synthetic construct
SRLEEELRRRLTE

SEQ ID NO: 86 - protein, 13 aa, synthetic construct
GRLEEELRRRLSP

SEQ ID NO: 87 - protein, 13 aa, synthetic construct
GRLEQEIRARLSP

SEQ ID NO: 88 - protein, 792 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDELVDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 89 - protein, 1005 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAGGSGGSGGSGGSAAAQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDELVDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 90 - protein, 999 aa, synthetic construct; identical to SEQ ID NO: 23

SEQ ID NO: 91 - protein, 794 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSTGAPPNLWAAQRYGRELRRMSDEFVDSFKKASQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 92 - protein, 993 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSTGAPPNLWAAQRYGRELRRMSDEFVDSFKKASQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSAAASSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 93 - protein, 1081 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDEFVDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGGTENLYFQSAASSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNGSSELIKENMHMKRPSVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKDYKDDDDKGSGATNFSLLKQAGDVEENPGPASAGSGEGRGSLLTCGDVEENPGPATGNSA

SEQ ID NO: 94 - protein, 1081 aa, synthetic construct; identical to SEQ ID NO: 93

SEQ ID NO: 95 - protein, 385 aa, synthetic construct
QLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 96 - protein, 385 aa, synthetic construct
QLHLPQVLADAVSRLVLGKFGDLTDNFSSPHARRKVLAGVVMTTGTDVKDAKVISVSTGTKCINGEYMSDRGLALNDCHAAIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 97 - protein, 153 aa, synthetic construct
QLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPA

SEQ ID NO: 98 - protein, 232 aa, synthetic construct
DRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 99 - DNA, 5372 bp, synthetic construct
  source 1..5372: mol_type = other DNA
cttatcgaaattaatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcgggtcgggcaccggtgctccacccaatctctgggcagcgcagcgctacggccgtgagctcagaaggatgtccgatgagttcgtcgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggaggtacggagaatttgtattttcagagcgccgcttcaagtaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatggatccagcgagctgattaaggagaacatgcacatgaagcgcccatcggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcgaaacatgaggatcacccatgtgccgctatggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttctggcaccggtatggcatctatgactggaggccaacagatgggtcctgcaaccgggaattccgcgtagcgctagctttgccagcgccacgcgaaacatgaggatcacccatgtactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctatcgatgtgagcaagggcgaagaagataacaaggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcag

SEQ ID NO: 100 - protein, 117 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 101 - protein, 121 aa, synthetic construct
MSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLG

SEQ ID NO: 102 - protein, 23 aa, synthetic construct
MADAQTRRRERRAEKQAQWKAAN

SEQ ID NO: 103 - protein, 19 aa, synthetic construct
MASGPRPRGTRGKGRRIRR

SEQ ID NO: 104 - protein, 305 aa, synthetic construct
LCQRHAKHEDHPCTSATNFSLLKQAGDVEENPGPGGSEGRGSLLTCGDVEENPGPSGYPYDVPDYAHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKAS

SEQ ID NO: 105 - DNA, 52 bp, synthetic construct
  source 1..52: mol_type = other DNA
cgcgtagcgctagctttgccagcgccacgcgaaacatgaggatcacccatgt

SEQ ID NO: 106 - DNA, 34 bp, synthetic construct
  source 1..34: mol_type = other DNA
cgcgtggcgctggcttccttgccagcgccacgcg

SEQ ID NO: 107 - DNA, 34 bp, synthetic construct
  source 1..34: mol_type = other DNA
cgcgtagcgctggcttccttgccagcgccacgcg

SEQ ID NO: 108 - DNA, 34 bp, synthetic construct
  source 1..34: mol_type = other DNA
cgcgtagcgctagcttccttgccagcgccacgcg

SEQ ID NO: 109 - DNA, 58 bp, synthetic construct
  source 1..58: mol_type = other DNA
cgcgtagcgctagctttgccagcgccacgcgaaggagcagacgatatggcgtcgctcc

SEQ ID NO: 110 - DNA, 54 bp, synthetic construct
  source 1..54: mol_type = other DNA
cgcgtagcgctagctttgccagcgccacgcggtaagggccctgaagaagggccc

SEQ ID NO: 111 - DNA, 63 bp, synthetic construct
  source 1..63: mol_type = other DNA
cgcgtagcgctagctttgccagcgccacgcggtaggctcgtctgagctcattagctccgagcc

SEQ ID NO: 112 - protein, 43 aa, unidentified
ATNFSLLKQAGDVEENPGPASAGSGEGRGSLLTCGDVEENPGP

SEQ ID NO: 113 - protein, 301 aa, synthetic construct
RWLPCQRHATSATNFSLLKQAGDVEENPGPGGSEGRGSLLTCGDVEENPGPSGYPYDVPDYAHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKAS

SEQ ID NO: 114 - protein, 299 aa, synthetic construct
LPCQRHATSATNFSLLKQAGDVEENPGPGGSEGRGSLLTCGDVEENPGPSGYPYDVPDYAHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKAS

SEQ ID NO: 115 - protein, 30 aa, synthetic construct
ASGSGTGAPPNLWAAQRYGRELRRMSDEFV

SEQ ID NO: 116 - protein, 28 aa, synthetic construct
APPNLWAAQRYGRELRRMSDEFVDSFKK

SEQ ID NO: 117 - protein, 195 aa, synthetic construct
SNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNN

SEQ ID NO: 118 - protein, 35 aa, synthetic construct
ASGSGSGDMRPEIWIAQELRRIGDEFNAYYARRTG

SEQ ID NO: 119 - protein, 35 aa, synthetic construct
ASGGSGGSGRPEIWMTQGLRRLGDEANAYYARRTG

SEQ ID NO: 120 - protein, 156 aa, synthetic construct
DELYRQSLEIISRYLREQATGAKDTKPMGRSGATSRKALETLRRVGDGVQRNHETAFQGMLRKLDIKNEDDVKSLSRVMIHVFSDGVTNWGRIVTLISFGAFVAKHLKTINQESCIEPLAESITDVLVRTKRDWLVKQRGWDGFVEFFHVEDLEGG

SEQ ID NO: 121 - protein, 30 aa, synthetic construct
ASGSGTGAPPNLWAAQRYGRELRRMSDELV

SEQ ID NO: 122 - protein, 14 aa, synthetic construct
SRLEEELRRRLTEP

SEQ ID NO: 123 - protein, 122 aa, synthetic construct
EVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSS

SEQ ID NO: 124 - protein, 18 aa, synthetic construct
GSGPGRLEEELRRRLSPG

SEQ ID NO: 125 - protein, 21 aa, synthetic construct
ASGSGPGRLEQEIRARLSPGT

SEQ ID NO: 126 - protein, 22 aa, synthetic construct
ASGGSGAHIVMVDAYKPTKGTG

SEQ ID NO: 127 - protein, 115 aa, synthetic construct
MVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIG

SEQ ID NO: 128 - protein, 242 aa, synthetic construct
VIPDYFKQSFPEGYSWERSMTYEDGGICIATNDITMEGDSFINKIHFKGTNFPPNGPVMQKRTVGWEASTEKMYERDGVLKGDVKMKLLLKGGGHYRCDYRTTYKVKQKPVKLPDYHFVDHRIEILSHDKDYNKVKLYEHAVARNSTDSMDELYKGGSGGMVSKGEETITSVIKPDMKNKLRMEGNVNGHAFVIEGEGSGKPFEGIQTIDLEVKEGAPLPFAYDILTTAFHYGNRVFTKYPR

SEQ ID NO: 129 - protein, 9 aa, synthetic construct
GTENLYFQS

SEQ ID NO: 130 - protein, 18 aa, synthetic construct
GSGGTENLYFQSGTSGGA

SEQ ID NO: 131 - DNA, 25 bp, synthetic construct
  source 1..25: mol_type = other DNA
ggagcagacgatatggcgtcgctcc

SEQ ID NO: 132 - DNA, 19 bp, synthetic construct
  source 1..19: mol_type = other DNA
gggccctgaagaagggccc

SEQ ID NO: 133 - DNA, 29 bp, synthetic construct
  source 1..29: mol_type = other DNA
ggctcgtctgagctcattagctccgagcc

SEQ ID NO: 134 - protein, 31 aa, synthetic construct
GALFDKSCSDRAMESTESRHYPVFENPKQGK

SEQ ID NO: 135 - protein, 26 aa, synthetic construct
ARIFSPHEPILEEPADRHPNRKARGQ

SEQ ID NO: 136 - protein, 26 aa, synthetic construct
ARLHSPYEITTDLHSSKHLVRKFRGH

SEQ ID NO: 137 - protein, 32 aa, synthetic construct
AQIKSQLRLNPHSISAFEANEELCLHVAVEGK

SEQ ID NO: 138 - protein, 30 aa, synthetic construct
AARDIYLPPTSEGGLPHSPPMRLQAHVLGQ

SEQ ID NO: 139 - protein, 10 aa, synthetic construct
KSCSDRAMES

SEQ ID NO: 140 - protein, 13 aa, synthetic construct
FSPHEPILEEPAD

SEQ ID NO: 141 - protein, 11 aa, synthetic construct
PYEITTDLHSS

SEQ ID NO: 142 - protein, 6 aa, synthetic construct
QLRLNP

SEQ ID NO: 143 - protein, 9 aa, synthetic construct
PPTSEGGLP

SEQ ID NO: 144 - protein, 305 aa, synthetic construct; identical to SEQ ID NO: 104

SEQ ID NO: 145 - protein, 305 aa, synthetic construct; identical to SEQ ID NO: 104

SEQ ID NO: 146 - protein, 308 aa, synthetic construct
LCQRHAKEQTIWRRSNTSATNFSLLKQAGDVEENPGPGGSEGRGSLLTCGDVEENPGPSGYPYDVPDYAHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKAS

SEQ ID NO: 147 - protein, 306 aa, synthetic construct
LCQRHAVRALKKGPTSATNFSLLKQAGDVEENPGPGGSEGRGSLLTCGDVEENPGPSGYPYDVPDYAHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKAS

SEQ ID NO: 148 - protein, 309 aa, synthetic construct
LCQRHAVGSSELISSEPTSATNFSLLKQAGDVEENPGPGGSEGRGSLLTCGDVEENPGPSGYPYDVPDYAHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKAS

SEQ ID NO: 149 - protein, 69 aa, synthetic construct
LCQRHTKHEDHPCRPHSSGAGCLSEGGGWCGQCPGSQIPLRSFSLCQKLWGHHEAPWASDFWLIKEIYF

SEQ ID NO: 150 - protein, 556 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDEFVDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 151 - protein, 765 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDEFVDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSAAGGSGGSAAASSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNN

SEQ ID NO: 152 - protein, 561 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGSGDMRPEIWIAQELRRIGDEFNAYYARRTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 153 - protein, 561 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGGSGGSGRPEIWMTQGLRRLGDEANAYYARRTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 154 - protein, 765 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDELVDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSAAGGSGGSAAASSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNN

SEQ ID NO: 155 - protein, 725 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGGSGGSGRPEIWMTQGLRRLGDEINAYYARRTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGTGGPGDELYRQSLEIISRYLREQATGAKDTKPMGRSGATSRKALETLRRVGDGVQRNHETAFQGMLRKLDIKNEDDVKSLSRVMIHVFSDGVTNWGRIVTLISFGAFVAKHLKTINQESCIEPLAESITDVLVRTKRDWLVKQRGWDGFVEFFHVEDLEGG

SEQ ID NO: 156 - protein, 545 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASPSRLEEELRRRLTEPTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 157 - protein, 673 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASPSRLEEELRRRLTEPTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGGTAEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSS

SEQ ID NO: 158 - protein, 548 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGPGRLEEELRRRLSPGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 159 - protein, 676 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGPGRLEEELRRRLSPGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGGTAEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSS

SEQ ID NO: 160 - protein, 715 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGGSGAHIVMVDAYKPTKGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSTSATNFSLLKQAGDVEENPGPGGSEGRGSLLTCGDVEENPGPGTSGGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIG

SEQ ID NO: 161 - protein, 680 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGGSGAHIVMVDAYKPTKGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGGTENLYFQSGTSGGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 162 - protein, 1004 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDELVDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSGGVIPDYFKQSFPEGYSWERSMTYEDGGICIATNDITMEGDSFINKIHFKGTNFPPNGPVMQKRTVGWEASTEKMYERDGVLKGDVKMKLLLKGGGHYRCDYRTTYKVKQKPVKLPDYHFVDHRIEILSHDKDYNKVKLYEHAVARNSTDSMDELYKGGSGGMVSKGEETITSVIKPDMKNKLRMEGNVNGHAFVIEGEGSGKPFEGIQTIDLEVKEGAPLPFAYDILTTAFHYGNRVFTKYPRSGSGSSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNN

SEQ ID NO: 163 - protein, 556 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDELVDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 164 - protein, 769 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAGGSGGSGGSGGSAAAQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDELVDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 165 - protein, 766 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDELVDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSAAGGSGGSAAASSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNG

SEQ ID NO: 166 - protein, 558 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSTGAPPNLWAAQRYGRELRRMSDEFVDSFKKASQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 167 - protein, 759 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSTGAPPNLWAAQRYGRELRRMSDEFVDSFKKASQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSAAASSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNN

SEQ ID NO: 168 - protein, 913 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYANFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPASSGGELDELVYLLDGPGYDPIHCDVVTRGGSHLFNFDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 169 - protein, 1125 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYANFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPASSGGELDELVYLLDGPGYDPIHCDVVTRGGSHLFNFDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSAAGGSGGSAAAQGSVVIVGRIILSGSGSITAYSQQTRGLLGCIITSLTGRDKNQVEGEVQVVSTATQSFLATCVNGVCWTVYHGAGSKTLAGPKGPITQMYTNVDQDLVGWQAPPGARSLTPCTCGSSDLYLVTRHADVIPVRRRGDSRGSLLSPRPVSYLKGSSGGPLLCPSGHAVGIFRAAVCTRGVAKAVDFVPVESMETTMRSESGSGTMSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 170 - DNA, 63 bp, synthetic construct
  source 1..63: mol_type = other DNA
aattccgcgtagcgctagctttgccagcgccacgcgaaacatgaggatcacccatgtactagt

SEQ ID NO: 171 - RNA, 63 nt, synthetic construct
  source 1..63: mol_type = other RNA
aattccgcgtagcgctagctttgccagcgccacgcgaaacatgaggatcacccatgtactagt

SEQ ID NO: 172 - protein, 15 aa, synthetic construct
LCQRHAKHEDHPCTS

SEQ ID NO: 173 - DNA, 2035 bp, synthetic construct
  source 1..2035: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcgaaacatgaggatcacccatgtactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctcatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttcta

SEQ ID NO: 174 - DNA, 63 bp, synthetic construct
  source 1..63: mol_type = other DNA
aattccgcgtagcgctggctttgccagcgccacgcgaaacatgaggatcacccatgtactagt

SEQ ID NO: 175 - RNA, 63 nt, synthetic construct
  source 1..63: mol_type = other RNA
aattccgcgtagcgctggctttgccagcgccacgcgaaacatgaggatcacccatgtactagt

SEQ ID NO: 176 - protein, 17 aa, synthetic construct
RWLCQRHAKHEDHPCTS

SEQ ID NO: 177 - DNA, 2034 bp, synthetic construct
  source 1..2034: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctggctttgccagcgccacgcgaaacatgaggatcacccatgtactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctcatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttct

SEQ ID NO: 178 - DNA, 63 bp, synthetic construct
  source 1..63: mol_type = other DNA
aattccgcgtggcgctagctttgccagcgccacgcgaaacatgaggatcacccatgtactagt

SEQ ID NO: 179 - RNA, 63 nt, synthetic construct
  source 1..63: mol_type = other RNA
aattccgcgtggcgctagctttgccagcgccacgcgaaacatgaggatcacccatgtactagt

SEQ ID NO: 180 - protein, 5 aa, synthetic construct
NSAWR

SEQ ID NO: 181 - DNA, 2034 bp, synthetic construct
  source 1..2034: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtggcgctagctttgccagcgccacgcgaaacatgaggatcacccatgtactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctcatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttct

SEQ ID NO: 182 - DNA, 60 bp, synthetic construct
  source 1..60: mol_type = other DNA
aattccgcgtagcgctagctacatgaggatcacccatgttgccagcgccacgcgactagt

SEQ ID NO: 183 - RNA, 60 nt, synthetic construct
  source 1..60: mol_type = other RNA
aattccgcgtagcgctagctacatgaggatcacccatgttgccagcgccacgcgactagt

SEQ ID NO: 184 - protein, 14 aa, synthetic construct
LHEDHPCCQRHATS

SEQ ID NO: 185 - DNA, 2031 bp, synthetic construct
  source 1..2031: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctacatgaggatcacccatgttgccagcgccacgcgactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctcatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttct

SEQ ID NO: 186 - DNA, 72 bp, synthetic construct
  source 1..72: mol_type = other DNA
aattccgcgtagcgctagctttgccagcgccacgcgaaggagcagacgatatggcgtcgctccaatactagt

SEQ ID NO: 187 - RNA, 72 nt, synthetic construct
  source 1..72: mol_type = other RNA
aattccgcgtagcgctagctttgccagcgccacgcgaaggagcagacgatatggcgtcgctccaatactagt

SEQ ID NO: 188 - protein, 18 aa, synthetic construct
LCQRHAKEQTIWRRSNTS

SEQ ID NO: 189 - DNA, 2043 bp, synthetic construct
  source 1..2043: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcgaaggagcagacgatatggcgtcgctccaatactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctcatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttct

SEQ ID NO: 190 - DNA, 75 bp, synthetic construct
  source 1..75: mol_type = other DNA
aattccgcgtagcgctagctttgccagcgccacgcggtaggctcgtctgagctcattagctccgagccaactagt

SEQ ID NO: 191 - RNA, 75 nt, synthetic construct
  source 1..75: mol_type = other RNA
aattccgcgtagcgctagctttgccagcgccacgcggtaggctcgtctgagctcattagctccgagccaactagt

SEQ ID NO: 192 - protein, 19 aa, synthetic construct
LCQRHAVGSSELISSEPTS

SEQ ID NO: 193 - DNA, 2046 bp, synthetic construct
  source 1..2046: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcggtaggctcgtctgagctcattagctccgagccaactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctcatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttct

SEQ ID NO: 194 - DNA, 66 bp, synthetic construct
  source 1..66: mol_type = other DNA
aattccgcgtagcgctagctttgccagcgccacgcggtaagggccctgaagaagggcccaactagt

SEQ ID NO: 195 - RNA, 66 nt, synthetic construct
  source 1..66: mol_type = other RNA
aattccgcgtagcgctagctttgccagcgccacgcggtaagggccctgaagaagggcccaactagt

SEQ ID NO: 196 - protein, 16 aa, synthetic construct
LCQRHAVRALKKGPTS

SEQ ID NO: 197 - DNA, 2037 bp, synthetic construct
  source 1..2037: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcggtaagggccctgaagaagggcccaactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctcatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttct

SEQ ID NO: 198 - protein, 1005 aa, synthetic construct; identical to SEQ ID NO: 89

SEQ ID NO: 199 - DNA, 3121 bp, synthetic construct
  source 1..3121: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagagtaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcggccggaggtagcggcggaagcggtggctctggaggctcagcggccgctcaattacacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcgggtcgggcaccggtgctccacccaatctctgggcagcgcagcgctacggccgtgagctcagaaggatgtccgatgagctggtcgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 200 - protein, 794 aa, synthetic construct; identical to SEQ ID NO: 91

SEQ ID NO: 201 - DNA, 2488 bp, synthetic construct
  source 1..2488: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctaccggtgctccacccaatctctgggcagcgcagcgctacggccgtgagctcagaaggatgtccgatgagttcgtcgattccttcaaaaaggctagccagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 202 - protein, 993 aa, synthetic construct; identical to SEQ ID NO: 92

SEQ ID NO: 203 - DNA, 3085 bp, synthetic construct
  source 1..3085: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctaccggtgctccacccaatctctgggcagcgcagcgctacggccgtgagctcagaaggatgtccgatgagttcgtcgattccttcaaaaaggctagccagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcgcggccgcctcaagtaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 204 - protein, 913 aa, synthetic construct; identical to SEQ ID NO: 168

SEQ ID NO: 205 - DNA, 2845 bp, synthetic construct
  source 1..2845: mol_type = other DNA
taatacgactcactatagggagacccaagctggctagaggatcgaacccttaaggccaccatggcgagcaattttacccagtttgtgcttgtggacaacggcggcaccggggacgtgacggtggccccctccaattttgccaatggcattgcagaatggataagctctaacagcaggagccaggcatacaaggtgacctgcagcgtgaggcagtcaagcgctcaaaacaggaagtacaccattaaggtcgaagtgcccaaaggagcttggaggtcttacctgaacatggaactgacaattcctatcttcgcgaccaatagcgactgtgagctgatcgtgaaggccatgcaaggcctgctgaaagacgggaatcccatacccagcgccatcgccgctaactcaggcatttacgctaatttcactcagttcgtactggttgacaatgggggaaccggcgacgttaccgtggctccaagcaacttcgctaacgggatcgccgagtggatcagcagtaattcacgctcccaagcctacaaagtaacctgctctgtacggcagagttcagcccagaaccgaaagtataccatcaaagtggaggtgccgaagggcgcctggcggagctatctgaatatggagctgaccatccccatctttgccacgaacagcgattgcgagctcatcgtcaaggcgatgcagggcttgctgaaggatggcaaccctatcccgagcgcaatagcagccaacagcggcatctatgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcgtcgtccggtggagaacttgatgaattggtatacttactagatgggccaggttatgaccctatacattgcgatgtagtgacaaggggcggcagccaccttttcaattttgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 206 - protein, 1125 aa, synthetic construct; identical to SEQ ID NO: 169

SEQ ID NO: 207 - DNA, 3481 bp, synthetic construct
  source 1..3481: mol_type = other DNA
taatacgactcactatagggagacccaagctggctagaggatcgaacccttaaggccaccatggcgagcaattttacccagtttgtgcttgtggacaacggcggcaccggggacgtgacggtggccccctccaattttgccaatggcattgcagaatggataagctctaacagcaggagccaggcatacaaggtgacctgcagcgtgaggcagtcaagcgctcaaaacaggaagtacaccattaaggtcgaagtgcccaaaggagcttggaggtcttacctgaacatggaactgacaattcctatcttcgcgaccaatagcgactgtgagctgatcgtgaaggccatgcaaggcctgctgaaagacgggaatcccatacccagcgccatcgccgctaactcaggcatttacgctaatttcactcagttcgtactggttgacaatgggggaaccggcgacgttaccgtggctccaagcaacttcgctaacgggatcgccgagtggatcagcagtaattcacgctcccaagcctacaaagtaacctgctctgtacggcagagttcagcccagaaccgaaagtataccatcaaagtggaggtgccgaagggcgcctggcggagctatctgaatatggagctgaccatccccatctttgccacgaacagcgattgcgagctcatcgtcaaggcgatgcagggcttgctgaaggatggcaaccctatcccgagcgcaatagcagccaacagcggcatctatgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcgtcgtccggtggagaacttgatgaattggtatacttactagatgggccaggttatgaccctatacattgcgatgtagtgacaaggggcggcagccaccttttcaattttgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcgcggccggaggtagcggcggaagcgcggccgctcaggggtctgttgttattgttggtagaattattttatctggtagtggtagtatcacggcctactcccaacagacgcggggcctacttggttgcatcatcactagcctcacaggccgggacaagaaccaggtcgaaggggaggttcaagtggtttctaccgcaacacaatctttcctggcgacctgcgtcaacggcgtgtgctggactgtctaccatggcgctggctcgaagaccctagccggtccaaaaggtccaatcacccaaatgtacaccaatgtagaccaggacctcgtcggctggcaggcgcctccaggggcgcgctccttgacaccatgcacctgtggcagctcggacctttacttggtcacgagacatgctgatgtcattccggtgcgccggcgaggcgacagcaggggaagtctactctcccccaggcccgtctcctacctgaaaggctcctcaggtggtccattgctttgcccttcggggcacgctgtgggcatcttccgggctgctgtgtgcacccggggggtcgcgaaggcggtggacttcgtgcccgttgagtctatggaaactaccatgcggtctgagagtggatcaggtaccatgagcgagctgattaaggagaacatgcacatgaagctgtacatggagggcaccgtgaacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 208 - protein, 189 aa, Hepacivirus hominis
APITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSS

SEQ ID NO: 209 - protein, 186 aa, Hepacivirus hominis
APITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTD

SEQ ID NO: 210 - protein, 180 aa, Hepacivirus hominis
APITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMR

SEQ ID NO: 211 - protein, 180 aa, Hepacivirus hominis
APITAYSQQTRGLLGCIITSLTGRDKNQVEGEVQVVSTATQSFLATCVNGVCWTVYHGAGSKTLAGPKGPITQMYTNVDQDLVGWQAPPGARSLTPCTCGSSDLYLVTRHADVIPVRRRGDSRGSLLSPRPVSYLKGSSGGPLLCPSGHAVGIFRAAVCTRGVAKAVDFVPVESMETTMR

SEQ ID NO: 212 - protein, 180 aa, Hepacivirus hominis
APITAYAQQTRGLLGTIVVSMTGRDKTEQAGEIQVLSTVTQSFLGTSISGVLWTVYHGAGNKTLAGSRGPVTQMYSSAEGDLVGWPSPPGTKSLEPCTCGAVDLYLVTRNADVIPARRRGDKRGALLSPRPLSTLKGSSGGPVLCPRGHAVGVFRAAVCSRGVAKSIDFIPVETLDIVTR

SEQ ID NO: 213 - protein, 180 aa, Hepacivirus hominis
APITAYAQQTRGLLGTIVTSLTGRDKNVVTGEVQVLSTATQTFLGTTVGGVIWTVYHGAGSRTLAGAKHPALQMYTNVDQDLVGWPAPPGAKSLEPCACGSSDLYLVTRDADVIPARRRGDSTASLLSPRPLACLKGSSGGPVMCPSGHVAGIFRAAVCTRGVAKSLQFIPVETLSTQAR

SEQ ID NO: 214 - protein, 180 aa, Hepacivirus hominis
APITAYAQQTRGLFSTIVTSLTGRDTNENCGEVQVLSTATQSFLGTAVNGVMWTVYHGAGAKTISGPKGPVNQMYTNVDQDLVGWPAPPGVRSLAPCTCGSADLYLVTRHADVIPVRRRGDTRGALLSPRPISILKGSSGGPLLCPMGHRAGIFRAAVCTRGVAKAVDFVPVESLETTMR

SEQ ID NO: 215 - protein, 180 aa, Hepacivirus hominis
APITAYAQQTRGVLGAIVLSLTGRDKNEAEGEVQFLSTATQTFLGICINGVMWTLFHGAGSKTLAGPKGPVVQMYTNVDKDLVGWPSPPGKGSLTRCTCGSADLYLVTRHADVIPARRRGDTRASLLSPRPISYLKGSSGGPIMCPSGHVVGVFRAAVCTRGVAKALEFVPVENLETTMR

SEQ ID NO: 216 - protein, 180 aa, Hepacivirus hominis
APITAYAQQTRGLVGTIVTSLTGRDKNEAEGEVQVVSTATQSFLATTINGVLWTVYHGAGSKNLAGPKGPVCQMYTNVDQDLVGWPAPLGARSLAPCTCGSSDLYLVTRGADVIPARRRGDTRAALLSPRPISTLKGSSGGPLMCPSGHVVGLFRAAVCTRGVAKALDFIPVENMDTTMR

SEQ ID NO: 217 - protein, 180 aa, Hepacivirus hominis
APISAYAQQTRGLISTLVVSLTGRDKNETAGEVQVLSTSTQTFLGTNVGGVMWGPYHGAGTRTVAGRGGPVLQMYTSVSDDLVGWPAPPGSKSLEPCSCGSADLYLVTRNADVLPLRRKGDGTASLLSPRPVSSLKGSSGGPVLCPQSHCVGIFRAAVCTRGVAKAVQFVPIEKMQVAQR

SEQ ID NO: 218 - protein, 180 aa, Hepacivirus hominis
APITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVTKAVDFIPVENLETTMR

SEQ ID NO: 219 - protein, 180 aa, Hepacivirus hominis
APITAYSQQTRGLLGCIITSLTGRDKNQVEGEVQVVSTATQSFLATCVNGVCWTVYHGAGSKTLAAPKGPITQMYTNVDQDLVGWPKPPGARSLTPCTCGSSDLYLVTRHADVIPVRRRGDSRGSLLSPRPVSYLKGSSGGPLLCPFGHAVGIFRAAVCTRGVAKAVDFVPVESMETTMR

SEQ ID NO: 220 - protein, 180 aa, Hepacivirus hominis
APITAYAQQTRGLLGTIVVSMTGRDKTEQAGEIQVLSTVTQSFLGTTISGVLWTVYHGAGNKTLAGSRGPVTQMYSSAEGDLVGWPSPPGTKSLEPCTCGAVDLYLVTRNADVIPARRRGDKRGALLSPRPLSTLKGSSGGPVLCPRGHAVGVFRAAVCSRGVAKSIDFIPVETLDIVTR

SEQ ID NO: 221 - protein, 180 aa, Hepacivirus hominis
APITAYTQQTRGLLGAIVVSLTGRDKNEQAGQVQVLSSVTQTFLGTSISGVLWTVYHGAGNKTLAGPKGPVTQMYTSAEGDLVGWPSPPGTKSLDPCTCGAVDLYLVTRNADVIPVRRKDDRRGALLSPRPLSTLKGSSGGPVLCSRGHAVGLFRAAVCARGVAKSIDFIPVESLDVATR

SEQ ID NO: 222 - protein, 180 aa, Hepacivirus hominis; identical to SEQ ID NO: 213

SEQ ID NO: 223 - protein, 186 aa, synthetic construct
APITAYAQQTRGLLGCIITSLTGRDKNQVEGEVQIMSTATQTFLATCINGVCWAVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTD

SEQ ID NO: 224 - protein, 186 aa, synthetic construct
APITAYAQQTRGLLGCIITSLTGRDKNQVEGEVQIVSTATQTLLATCINGVCWAVYHGAGTRTIASPKGPVIQMYTNVDKDLVGWPAPQGSRSLTPCTCGSSDLYLVTRHADVIPVRRRGDRRGSLLSPRPISYLKGSSGGPLLCPAGHAVGLFRAAVCTRGVAKAVYFIPVENLETTMRSPVFTD

SEQ ID NO: 225 - protein, 13 aa, Hepacivirus hominis
GCVVIVGRIVLSG

SEQ ID NO: 226 - protein, 45 aa, Hepacivirus hominis
STWVLVGGVLAALAAYCLSTGCVVIVGRIVLSGKPAIIPDREVLY

SEQ ID NO: 227 - protein, 51 aa, Hepacivirus hominis
STWVLVGGVLAALAAYCLSTGCVVIVGRIVLSGKPAGSSGSSIIPDREVLY

SEQ ID NO: 228 - protein, 63 aa, Hepacivirus hominis
IDTKYIMTCMSADLEVVTSTWVLVGGVLAALAAYCLSTGCVVIVGRIVLSGKPAIIPDREVLY

SEQ ID NO: 229 - protein, 12 aa, Hepacivirus hominis
GSVVIVGRIILS

SEQ ID NO: 230 - protein, 14 aa, Hepacivirus hominis
GCVVIVGRIVLSGK

SEQ ID NO: 231 - protein, 14 aa, Hepacivirus hominis
GCVCIIGRLHINQR

SEQ ID NO: 232 - protein, 14 aa, Hepacivirus hominis
GCVVIVGHIELEGK

SEQ ID NO: 233 - protein, 14 aa, Hepacivirus hominis
GSVVIVGRVVLSGQ

SEQ ID NO: 234 - protein, 14 aa, Hepacivirus hominis
GSVAIVGRIILSGR

SEQ ID NO: 235 - protein, 14 aa, Hepacivirus hominis
GCVVICGRIVTSGK

SEQ ID NO: 236 - protein, 14 aa, Hepacivirus hominis
GSVVVVGRVVLGSN

SEQ ID NO: 237 - protein, 54 aa, Hepacivirus hominis
STWVLVGGVLAALAAYCLSTGCVVIVGRIVLSGKPAIIPDREVLYQEFDEMEEC

SEQ ID NO: 238 - protein, 54 aa, Hepacivirus hominis
STWVLVGGVLAALAAYCLSTGCVVIVGRVVLSGKPAIIPDREVLYREFDEMEEC

SEQ ID NO: 239 - protein, 54 aa, Hepacivirus hominis
STWVLVGGVLAALAAYCLSTGCVVIVGRIVLSGRPAIIPDREVLYREFDEMEEC

SEQ ID NO: 240 - protein, 54 aa, Hepacivirus hominis
STWVLVGGVLAALAAYCLTTGSVVIVGRIILSGRPAIVPDRELLYQEFDEMEEC

SEQ ID NO: 241 - protein, 54 aa, Hepacivirus hominis
STWVLVGGVLAALAAYCLTTGSVVIVGRIILSGRPAIIPDRELLYQEFDEMEEC

SEQ ID NO: 242 - protein, 54 aa, Hepacivirus hominis
STWVLVGGVLAALAAYCLTTGSVVIVGRIILSGKPAVVPDRELLYQEFDEMEEC

SEQ ID NO: 243 - protein, 54 aa, Hepacivirus hominis
STWVLVGGVLAALAAYCLTTGSVVIVGRIILSGRPAVIPDRELLYREFDEMEEC

SEQ ID NO: 244 - protein, 54 aa, Hepacivirus hominis
STWVLAGGVLAAVAAYCLATGCVCIIGRLHVNQRAVVAPDKEVLYEAFDEMEEC

SEQ ID NO: 245 - protein, 54 aa, Hepacivirus hominis
STWVLAGGVLAAVAAYCLATGCVSIIGRLHINGRAVVAPDKEVLYEAFDEMEEC

SEQ ID NO: 246 - protein, 54 aa, Hepacivirus hominis
SSWVLAGGVLAAVAAYCLATGCISIIGRLHLNDRVVVAPDKEILYEAFDEMEEC

SEQ ID NO: 247 - protein, 54 aa, Hepacivirus hominis
STWVLAGGVLAAVAAYCLATGCVSIIGRLHLNDQVVVTPDKEILYEAFDEMEEC

SEQ ID NO: 248 - protein, 54 aa, Hepacivirus hominis
STWVLLGGVLAALAAYCLSVGCVVIVGHIELEGKPALVPDKEVLYQQYDEMEEC

SEQ ID NO: 249 - protein, 54 aa, Hepacivirus hominis
STWVLLGGVLAAVAAYCLSVGCVVIVGHIELGGKPALVPDKEVLYQQYDEMEEC

SEQ ID NO: 250 - protein, 4 aa, synthetic construct
SGTS

SEQ ID NO: 251 - protein, 4 aa, synthetic construct
GSGS

SEQ ID NO: 252 - protein, 182 aa, synthetic construct
MAPITAYAQQTRGLLGCIITSLTGRDKNQVEGEVQIVSTAAQTFLATCINGVCWTVYHGAGTRTIASPKGPVIQMYTNVDKDLVGWPAPQGSRSLTPCTCGSSDLYLVTRHADVIPVRRRGDSRGSLLSPRPISYLKGSSGGPLLCPAGHAVGIFRAAVCTRGVAKAVDFIPVESLETTMRS

SEQ ID NO: 253 - protein, 195 aa, synthetic construct
MKKKGSVVIVGRIVLNGAYAQQTRGLLGCIITSLTGRDKNQVEGEVQIVSTAAQTFLATCINGVCWTVYHGAGTRTIASPKGPVIQMYTNVDKDLVGWPAPQGSRSLTPCTCGSSDLYLVTRHADVIPVRRRGDSRGSLLSPRPISYLKGSSGGPLLCPAGHAVGIFRAAVCTRGVAKAVDFIPVESLETTMRSP

SEQ ID NO: 254 - protein, 195 aa, synthetic construct
MKKKGSVVIVGRIVLNGAYAQQTRGEEGCQETSQTGRDKNQVEGEVQIVSTAAQTFLATCINGVCWTVYHGAGTRTIASPKGPVIQMYTNVDKDLVGWPAPQGSRSLTPCTCGSSDLYLVTRHADVIPVRRRGDSRGSLLSPRPISYLKGSSGGPLLCPAGHAVGIFRAAVCTRGVAKAVDFIPVESLETTMRSP

SEQ ID NO: 255 - protein, 197 aa, synthetic construct
MKKKGSVVIVGRINLSGDTAYAQQTRGEEGCQETSQTGRDKNQVEGEVQIVSTAAQTFLATCINGVCWTVYHGAGTRTIASPKGPVIQMYTNVDKDLVGWPAPQGSRSLTPCTCGSSDLYLVTRHADVIPVRRRGDSRGSLLSPRPISYLKGSSGGPLLCPAGHAVGIFRAAVCTRGVAKAVDFIPVESLETTMRSP

SEQ ID NO: 256 - protein, 197 aa, synthetic construct
MKKKGSVVIVGRINLSGDTAYAQQTRGEEGCQETSQTGRDKNQVEGEVQIVSTATQTFLATCINGVCWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVCTRGVAKAVDFIPVESLETTMRSP

SEQ ID NO: 257 - protein, 197 aa, synthetic construct
MKKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSP

SEQ ID NO: 258 - protein, 197 aa, synthetic construct
MKKKGSVVIVGRINLSGDTAYAQQTRGEQGCQKTSHTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSP

SEQ ID NO: 259 - protein, 197 aa, synthetic construct
MKKKGSVVIVGRINLSGDTAYAQQTRGEQGTQKTSHTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSP

SEQ ID NO: 260 - protein, 196 aa, synthetic construct
KKKGSVVIVGRINLSGDTAYAQQTRGEEGCQETSQTGRDKNQVEGEVQIVSTATQTFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSP

SEQ ID NO: 261 - protein, 21 aa, synthetic construct
GELDELVYLLDGPGYDPIHSD

SEQ ID NO: 262 - DNA, 537 bp, synthetic construct
  source 1..537: mol_type = other DNA
atcacggcctactcccaacagacgcggggcctacttggttgcatcatcactagcctcacaggccgggacaagaaccaggtcgaaggggaggttcaagtggtttctaccgcaacacaatctttcctggcgacctgcgtcaacggcgtgtgctggactgtctaccatggcgctggctcgaagaccctagccggtccaaaaggtccaatcacccaaatgtacaccaatgtagaccaggacctcgtcggctggcaggcgcctccaggggcgcgctccttgacaccatgcacctgtggcagctcggacctttacttggtcacgagacatgctgatgtcattccggtgcgccggcgaggcgacagcaggggaagtctactctcccccaggcccgtctcctacctgaaaggctccgcaggtggtccattgctttgcccttcggggcacgctgtgggcatcttccgggctgctgtgtgcacccggggggtcgcgaaggcggtggacttcgtgcccgttgagtctatggaaactaccatgcggtct

SEQ ID NO: 263 - protein, 179 aa, synthetic construct
ITAYSQQTRGLLGCIITSLTGRDKNQVEGEVQVVSTATQSFLATCVNGVCWTVYHGAGSKTLAGPKGPITQMYTNVDQDLVGWQAPPGARSLTPCTCGSSDLYLVTRHADVIPVRRRGDSRGSLLSPRPVSYLKGSAGGPLLCPSGHAVGIFRAAVCTRGVAKAVDFVPVESMETTMRS

SEQ ID NO: 264 - DNA, 567 bp, synthetic construct
  source 1..567: mol_type = other DNA
gcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctgggcagtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctccgcggggggtccgctgttgtgccccgcgggacacgccgtgggcctattcagggccgcggtgtgcacccgtggagtggctaaagcggtggactttatccctgtggagaacctagagacaaccatgagatccccggtgttcacggacaactcctct

SEQ ID NO: 265 - protein, 189 aa, synthetic construct
APITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSAGGPLLCPAGHAVGLFRAAVCTRGVAKAVDFIPVENLETTMRSPVFTDNSS

SEQ ID NO: 266 - protein, 33 aa, synthetic construct
GELGRLVYLLDGPGYDPIHCSLAYGDASTLVVF

SEQ ID NO: 267 - protein, 18 aa, synthetic construct
GELGRLVYLLDGPGYDPI

SEQ ID NO: 268 - protein, 15 aa, synthetic construct
HCSLAYGDASTLVVF

SEQ ID NO: 269 - protein, 33 aa, synthetic construct
GELGRPVYVLGDPGYYATHCIYATTNDALIFSV

SEQ ID NO: 270 - protein, 18 aa, synthetic construct
GELGRPVYVLGDPGYYAT

SEQ ID NO: 271 - protein, 15 aa, synthetic construct
HCIYATTNDALIFSV

SEQ ID NO: 272 - protein, 33 aa, synthetic construct
GELGRIPSDTYDLAVGALHCPFYLVSGLVYLDG

SEQ ID NO: 273 - protein, 33 aa, synthetic construct
GELGRLVYLLDGPGYDPIHCDVVTRGGSHLFNF

SEQ ID NO: 274 - protein, 33 aa, synthetic construct
GELDELVYLLDGPGYDPIHCDVVTRGGSHLFNF

SEQ ID NO: 275 - protein, 21 aa, synthetic construct
GELGRLVYLLDGPGYDPIHCD

SEQ ID NO: 276 - protein, 20 aa, synthetic construct
GELDELVYLLDGPGYDPIHS

SEQ ID NO: 277 - protein, 36 aa, synthetic construct
RQIKIWFQNRRMKWKKGELDELVYLLDGPGYDPIHS

SEQ ID NO: 278 - DNA, 99 bp, synthetic construct
  source 1..99: mol_type = other DNA
ggagaacttgatgaattggtatacttactagatgggccaggttatgaccctatacattgcgatgtagtgacaaggggcggcagccaccttttcaatttt

SEQ ID NO: 279 - protein, 677 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYANFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPASSGGELDELVYLLDGPGYDPIHCDVVTRGGSHLFNFDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 280 - protein, 888 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYANFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPASSGGELDELVYLLDGPGYDPIHCDVVTRGGSHLFNFDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSAAGGSGGSAAAQGSVVIVGRIILSGSGSITAYSQQTRGLLGCIITSLTGRDKNQVEGEVQVVSTATQSFLATCVNGVCWTVYHGAGSKTLAGPKGPITQMYTNVDQDLVGWQAPPGARSLTPCTCGSSDLYLVTRHADVIPVRRRGDSRGSLLSPRPVSYLKGSSGGPLLCPSGHAVGIFRAAVCTRGVAKAVDFVPVESMETTMRSES

SEQ ID NO: 281 - protein, 769 aa, synthetic construct; identical to SEQ ID NO: 164

SEQ ID NO: 282 - protein, 557 aa, synthetic construct
ASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSTGAPPNLWAAQRYGRELRRMSDEFVDSFKKASQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 283 - protein, 759 aa, synthetic construct; identical to SEQ ID NO: 167

SEQ ID NO: 284 - protein, 677 aa, synthetic construct; identical to SEQ ID NO: 279

SEQ ID NO: 285 - protein, 888 aa, synthetic construct; identical to SEQ ID NO: 280

SEQ ID NO: 286 - protein, 7 aa, synthetic construct
ENLYFQG

SEQ ID NO: 287 - protein, 999 aa, synthetic construct; identical to SEQ ID NO: 18

SEQ ID NO: 288 - protein, 999 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDEVVDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSAAGGSGGSAAASSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 289 - protein, 999 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDEGVDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSAAGGSGGSAAASSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 290 - protein, 797 aa, synthetic construct; identical to SEQ ID NO: 21

SEQ ID NO: 291 - protein, 797 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGGSGGSGRPEIWMTQGLRRLGDEGNAYYARRTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 292 - DNA, 2461 bp, synthetic construct
  source 1..2461: mol_type = other DNA
ctctagactgcagcctcaggagatctgggcccctacttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcaccatggtggcgaattctccaggcgatctgacggttcactaaacgagctctgcttatataggcctcccaccgtacacgccacctcgacatactcgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagggatcctctagtcagctgacgcgtgctagcgatatcggcgcgccagcactcaccatcgtcccgctgaggatcacccagcggccacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtaggcggccgcactcctcaggtgcaggctgcctatcagaaggtggtggctggtgtggccaatgccctggctcacaaataccactgagatctttttccctctgccaaaa

SEQ ID NO: 293 - DNA, 2461 bp, synthetic construct
  source 1..2461: mol_type = other DNA
ctctagactgcagcctcaggagatctgggcccctacttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcaccatggtggcgaattctccaggcgatctgacggttcactaaacgagctctgcttatataggcctcccaccgtacacgccacctcgacatactcgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagggatcctctagtcagctgacgcgtgctagcgatatcggcgcgccagcactcaccatcgtcccgctgaggatcacccagcggccacgatagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtaggcggccgcactcctcaggtgcaggctgcctatcagaaggtggtggctggtgtggccaatgccctggctcacaaataccactgagatctttttccctctgccaaaa

SEQ ID NO: 294 - DNA, 2461 bp, synthetic construct
  source 1..2461: mol_type = other DNA
ctctagactgcagcctcaggagatctgggcccctacttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcaccatggtggcgaattctccaggcgatctgacggttcactaaacgagctctgcttatataggcctcccaccgtacacgccacctcgacatactcgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagggatcctctagtcagctgacgcgtgctagcgatatcggcgcgccagcactcaccatcgtcccgctgaggataacccagcggccacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtaggcggccgcactcctcaggtgcaggctgcctatcagaaggtggtggctggtgtggccaatgccctggctcacaaataccactgagatctttttccctctgccaaaa

SEQ ID NO: 295 - DNA, 2461 bp, synthetic construct
  source 1..2461: mol_type = other DNA
ctctagactgcagcctcaggagatctgggcccctacttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcaccatggtggcgaattctccaggcgatctgacggttcactaaacgagctctgcttatataggcctcccaccgtacacgccacctcgacatactcgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagggatcctctagtcagctgacgcgtgctagcgatatcggcgcgccagcactcaccatcgtcccgctgaggataacccagcggccacgatagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtaggcggccgcactcctcaggtgcaggctgcctatcagaaggtggtggctggtgtggccaatgccctggctcacaaataccactgagatctttttccctctgccaaaa

SEQ ID NO: 296 - DNA, 1120 bp, synthetic construct
  source 1..1120: mol_type = other DNA
taatacgactcactatagggagacccaagctggctagcggtaccgccaccatggagaccgacaccctgctcctgtgggtgttgttgctctgggtcccaggttctaccggcgacggaggaggcggctatccctatgacgtaccggattatgccggtgaacttgatgaattggtatacttactagatgggccaggttatgaccctatacatagtggcggtagtgggggacgtacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggatgaactataccaactcgagaatggcggcataagcctgctggttcagaacacatcctggatgctgctgctgctgctttccctctccctcctccaagccctggacttcatttctctgtaatctagagggcccgtttaaacccgctgatcagcctcgactgtgccttctagttgccagccatctgt

SEQ ID NO: 297 - DNA, 1071 bp, synthetic construct
  source 1..1071: mol_type = other DNA
ttatcgaaattaatacgactcactatagggagacccaagctggctagctggtctccatcgtgcccgctgaggatcacccagcggccacgatagagaccgacaccctgctcctgtgggtgttgttgctctgggtcccaggttctaccggcgacggaggaggcggctatccctacgacgtaccggattacgccggtggcggtagtgggggacgtacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggatgaactataccaactcgagaatggcggcataagcctgctggttcagaacacatcctggatgctgctgctgctgctttccctctccctcctccaagccctggacttcatttctctgtaatctagagggcccgtttaaacccgctgatcagcct

SEQ ID NO: 298 - DNA, 1071 bp, synthetic construct
  source 1..1071: mol_type = other DNA
ttatcgaaattaatacgactcactatagggagacccaagctggctagctggtctccatcgtgcccgctgaggataacccagcggccacgatagagaccgacaccctgctcctgtgggtgttgttgctctgggtcccaggttctaccggcgacggaggaggcggctatccctacgacgtaccggattacgccggtggcggtagtgggggacgtacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggatgaactataccaactcgagaatggcggcataagcctgctggttcagaacacatcctggatgctgctgctgctgctttccctctccctcctccaagccctggacttcatttctctgtaatctagagggcccgtttaaacccgctgatcagcct

SEQ ID NO: 299 - DNA, 2461 bp, synthetic construct
  source 1..2461: mol_type = other DNA
ctctagactgcagcctcaggagatctgggcccctacttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcaccatggtggcgaattctccaggcgatctgacggttcactaaacgagctctgcttatataggcctcccaccgtacacgccacctcgacatactcgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagggatcctctagtcagctgacgcgtgctagcgatatcggcgcgccagcactcaccaccgtcccgctgaggatcacccagcggccacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtaggcggccgcactcctcaggtgcaggctgcctatcagaaggtggtggctggtgtggccaatgccctggctcacaaataccactgagatctttttccctctgccaaaa

SEQ ID NO: 300 - DNA, 2461 bp, synthetic construct
  source 1..2461: mol_type = other DNA
ctctagactgcagcctcaggagatctgggcccctacttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcaccatggtggcgaattctccaggcgatctgacggttcactaaacgagctctgcttatataggcctcccaccgtacacgccacctcgacatactcgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagggatcctctagtcagctgacgcgtgctagcgatatcggcgcgccagcactcaccaccgtcccgctgaggataacccagcggccacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtaggcggccgcactcctcaggtgcaggctgcctatcagaaggtggtggctggtgtggccaatgccctggctcacaaataccactgagatctttttccctctgccaaaa

SEQ ID NO: 301 - DNA, 2032 bp, synthetic construct
  source 1..2032: mol_type = other DNA
taatacgactcactatagggagacccaagctggctagcgtttaaacttaagcttgccaccatggcattgcccgtgaccgccctgctgctgccactggccttgttgctccacgccgcgcggccatatccctacgatgtgcccgattacgctaccggtatggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtccacgctcgagatttccggcggcggaggaagtggcgagggcaggggaagtctcctaacatgcggggacgtggaggaaaaccctaggccttacatgaggatcacccatgttaggcccagggttgactacaaagaccatgacggtgattataaagatcatgacatcgactacaaggacgacgatgacaagggtaccatggagggcagaggaagtcttctaacatgcggtgacgtggaggagaatcccggccctggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattgatctagagggcccgtttaaacccgctgatcagcctcgactgtgccttcta

SEQ ID NO: 302 - DNA, 3049 bp, synthetic construct
  source 1..3049: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcgaaacatgaggatcacccatgtgccgctatggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttctggcaccggtatggcatctatgactggaggccaacagatgggtcctgcaaccgggaattccgcgtagcgctagctttgccagcgccacgcgaaggagcagacgatatggcgtcgctccaatactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctatcgatgtgagcaagggcgaagaagataacaaggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttcta

SEQ ID NO: 303 - DNA, 3043 bp, synthetic construct
  source 1..3043: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcgaaacatgaggatcacccatgtgccgctatggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttctggcaccggtatggcatctatgactggaggccaacagatgggtcctgcaaccgggaattccgcgtagcgctagctttgccagcgccacgcggtaagggccctgaagaagggcccaactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctatcgatgtgagcaagggcgaagaagataacaaggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttcta

SEQ ID NO: 304 - DNA, 3052 bp, synthetic construct
  source 1..3052: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcgaaacatgaggatcacccatgtgccgctatggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttctggcaccggtatggcatctatgactggaggccaacagatgggtcctgcaaccgggaattccgcgtagcgctagctttgccagcgccacgcggtaggctcgtctgagctcattagctccgagccaactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctatcgatgtgagcaagggcgaagaagataacaaggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttcta

SEQ ID NO: 305 - DNA, 2065 bp, synthetic construct
  source 1..2065: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcgaaacatgaggatcacccatgtactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctcatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtaccttcaagtagagctacaccactggtgtagctccacttgataccatgaggatcacccatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttcta

SEQ ID NO: 306 - DNA, 3571 bp, synthetic construct
  source 1..3571: mol_type = other DNA
taatacgactcactatagggagacccaagctggctagaggatcgaacccttaaggccaccatggcgagcaattttacccagtttgtgcttgtggacaacggcggcaccggggacgtgacggtggccccctccaattttgccaatggcattgcagaatggataagctctaacagcaggagccaggcatacaaggtgacctgcagcgtgaggcagtcaagcgctcaaaacaggaagtacaccattaaggtcgaagtgcccaaaggagcttggaggtcttacctgaacatggaactgacaattcctatcttcgcgaccaatagcgactgtgagctgatcgtgaaggccatgcaaggcctgctgaaagacgggaatcccatacccagcgccatcgccgctaactcaggcatttacgctaatttcactcagttcgtactggttgacaatgggggaaccggcgacgttaccgtggctccaagcaacttcgctaacgggatcgccgagtggatcagcagtaattcacgctcccaagcctacaaagtaacctgctctgtacggcagagttcagcccagaaccgaaagtataccatcaaagtggaggtgccgaagggcgcctggcggagctatctgaatatggagctgaccatccccatctttgccacgaacagcgattgcgagctcatcgtcaaggcgatgcagggcttgctgaaggatggcaaccctatcccgagcgcaatagcagccaacagcggcatctatgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcccatcccgcctggaggaagaacttcggaggagacttactgagcctaccggctccggagccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgatgtggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctgtgacggggagcggaggtaccgccgaagttcaattacaggaatcgggtggaggtctggtacaacctgggggctctcttcgcctgagttgcactgccagtggagttacgatttctgcacttaatgctatggcgatgggttggtatcgtcaggccccaggggaacgtcgcgtcatggtcgctgccgtttccgaacgtggcaatgctatgtaccgcgagtctgttcagggccgcttcacggttacccgcgattttacaaataaaatggtatcgttgcaaatggacaacttaaagccagaggacactgctgtgtactactgtcacgtccttgaagatcgtgtggattcctttcatgattattgggggcaggggactcaggtcactgtatcctcaggagctggatccggaggtggaggctccggcaccatgatggatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtctccagcatgcatagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 307 - DNA, 3571 bp, synthetic construct
  source 1..3571: mol_type = other DNA
taatacgactcactatagggagacccaagctggctagaggatcgaacccttaaggccaccatggcgagcaattttacccagtttgtgcttgtggacaacggcggcaccggggacgtgacggtggccccctccaattttgccaatggcattgcagaatggataagctctaacagcaggagccaggcatacaaggtgacctgcagcgtgaggcagtcaagcgctcaaaacaggaagtacaccattaaggtcgaagtgcccaaaggagcttggaggtcttacctgaacatggaactgacaattcctatcttcgcgaccaatagcgactgtgagctgatcgtgaaggccatgcaaggcctgctgaaagacgggaatcccatacccagcgccatcgccgctaactcaggcatttacgctaatttcactcagttcgtactggttgacaatgggggaaccggcgacgttaccgtggctccaagcaacttcgctaacgggatcgccgagtggatcagcagtaattcacgctcccaagcctacaaagtaacctgctctgtacggcagagttcagcccagaaccgaaagtataccatcaaagtggaggtgccgaagggcgcctggcggagctatctgaatatggagctgaccatccccatctttgccacgaacagcgattgcgagctcatcgtcaaggcgatgcagggcttgctgaaggatggcaaccctatcccgagcgcaatagcagccaacagcggcatctatgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcccatcccgcctggaggaagaacttcggaggagacttactgagcctaccggctccggagccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgatgtggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctgggacggggagcggaggtaccgccgaagttcaattacaggaatcgggtggaggtctggtacaacctgggggctctcttcgcctgagttgcactgccagtggagttacgatttctgcacttaatgctatggcgatgggttggtatcgtcaggccccaggggaacgtcgcgtcatggtcgctgccgtttccgaacgtggcaatgctatgtaccgcgagtctgttcagggccgcttcacggttacccgcgattttacaaataaaatggtatcgttgcaaatggacaacttaaagccagaggacactgctgtgtactactgtcacgtccttgaagatcgtgtggattcctttcatgattattgggggcaggggactcaggtcactgtatcctcaggagctggatccggaggtggaggctccggcaccatgatggatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtctccagcatgcatagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 308 - DNA, 3571 bp, synthetic construct
  source 1..3571: mol_type = other DNA
taatacgactcactatagggagacccaagctggctagaggatcgaacccttaaggccaccatggcgagcaattttacccagtttgtgcttgtggacaacggcggcaccggggacgtgacggtggccccctccaattttgccaatggcattgcagaatggataagctctaacagcaggagccaggcatacaaggtgacctgcagcgtgaggcagtcaagcgctcaaaacaggaagtacaccattaaggtcgaagtgcccaaaggagcttggaggtcttacctgaacatggaactgacaattcctatcttcgcgaccaatagcgactgtgagctgatcgtgaaggccatgcaaggcctgctgaaagacgggaatcccatacccagcgccatcgccgctaactcaggcatttacgctaatttcactcagttcgtactggttgacaatgggggaaccggcgacgttaccgtggctccaagcaacttcgctaacgggatcgccgagtggatcagcagtaattcacgctcccaagcctacaaagtaacctgctctgtacggcagagttcagcccagaaccgaaagtataccatcaaagtggaggtgccgaagggcgcctggcggagctatctgaatatggagctgaccatccccatctttgccacgaacagcgattgcgagctcatcgtcaaggcgatgcagggcttgctgaaggatggcaaccctatcccgagcgcaatagcagccaacagcggcatctatgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcccatcccgcctggaggaagaacttcggaggagacttactgagcctaccggctccggagccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgatgtggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaatactctctgacggggagcggaggtaccgccgaagttcaattacaggaatcgggtggaggtctggtacaacctgggggctctcttcgcctgagttgcactgccagtggagttacgatttctgcacttaatgctatggcgatgggttggtatcgtcaggccccaggggaacgtcgcgtcatggtcgctgccgtttccgaacgtggcaatgctatgtaccgcgagtctgttcagggccgcttcacggttacccgcgattttacaaataaaatggtatcgttgcaaatggacaacttaaagccagaggacactgctgtgtactactgtcacgtccttgaagatcgtgtggattcctttcatgattattgggggcaggggactcaggtcactgtatcctcaggagctggatccggaggtggaggctccggcaccatgatggatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtctccagcatgcatagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 309 - DNA, 3568 bp, synthetic construct
  source 1..3568: mol_type = other DNA
taatacgactcactatagggagacccaagctggctagaggatcgaacccttaaggccaccatggcgagcaattttacccagtttgtgcttgtggacaacggcggcaccggggacgtgacggtggccccctccaattttgccaatggcattgcagaatggataagctctaacagcaggagccaggcatacaaggtgacctgcagcgtgaggcagtcaagcgctcaaaacaggaagtacaccattaaggtcgaagtgcccaaaggagcttggaggtcttacctgaacatggaactgacaattcctatcttcgcgaccaatagcgactgtgagctgatcgtgaaggccatgcaaggcctgctgaaagacgggaatcccatacccagcgccatcgccgctaactcaggcatttacgctaatttcactcagttcgtactggttgacaatgggggaaccggcgacgttaccgtggctccaagcaacttcgctaacgggatcgccgagtggatcagcagtaattcacgctcccaagcctacaaagtaacctgctctgtacggcagagttcagcccagaaccgaaagtataccatcaaagtggaggtgccgaagggcgcctggcggagctatctgaatatggagctgaccatccccatctttgccacgaacagcgattgcgagctcatcgtcaaggcgatgcagggcttgctgaaggatggcaaccctatcccgagcgcaatagcagccaacagcggcatctatgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggatcaggtccaggacgcctggaggaagaacttcggaggagactttctcctggaaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgatgtggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctgtgacggggagcggaggtaccgccgaagttcaattacaggaatcgggtggaggtctggtacaacctgggggctctcttcgcctgagttgcactgccagtggagttacgatttctgcacttaatgctatggcgatgggttggtatcgtcaggccccaggggaacgtcgcgtcatggtcgctgccgtttccgaacgtggcaatgctatgtaccgcgagtctgttcagggccgcttcacggttacccgcgattttacaaataaaatggtatcgttgcaaatggacaacttaaagccagaggacactgctgtgtactactgtcacgtccttgaagatcgtgtggattcctttcatgattattgggggcaggggactcaggtcactgtatcctcaggagctggatccggaggtggaggctccggcaccatgatggatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtctccagcatgcatagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 310 - DNA, 3568 bp, synthetic construct
  source 1..3568: mol_type = other DNA
taatacgactcactatagggagacccaagctggctagaggatcgaacccttaaggccaccatggcgagcaattttacccagtttgtgcttgtggacaacggcggcaccggggacgtgacggtggccccctccaattttgccaatggcattgcagaatggataagctctaacagcaggagccaggcatacaaggtgacctgcagcgtgaggcagtcaagcgctcaaaacaggaagtacaccattaaggtcgaagtgcccaaaggagcttggaggtcttacctgaacatggaactgacaattcctatcttcgcgaccaatagcgactgtgagctgatcgtgaaggccatgcaaggcctgctgaaagacgggaatcccatacccagcgccatcgccgctaactcaggcatttacgctaatttcactcagttcgtactggttgacaatgggggaaccggcgacgttaccgtggctccaagcaacttcgctaacgggatcgccgagtggatcagcagtaattcacgctcccaagcctacaaagtaacctgctctgtacggcagagttcagcccagaaccgaaagtataccatcaaagtggaggtgccgaagggcgcctggcggagctatctgaatatggagctgaccatccccatctttgccacgaacagcgattgcgagctcatcgtcaaggcgatgcagggcttgctgaaggatggcaaccctatcccgagcgcaatagcagccaacagcggcatctatgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggatcaggtccaggacgcctggaggaagaacttcggaggagactttctcctggaaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgatgtggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctgggacggggagcggaggtaccgccgaagttcaattacaggaatcgggtggaggtctggtacaacctgggggctctcttcgcctgagttgcactgccagtggagttacgatttctgcacttaatgctatggcgatgggttggtatcgtcaggccccaggggaacgtcgcgtcatggtcgctgccgtttccgaacgtggcaatgctatgtaccgcgagtctgttcagggccgcttcacggttacccgcgattttacaaataaaatggtatcgttgcaaatggacaacttaaagccagaggacactgctgtgtactactgtcacgtccttgaagatcgtgtggattcctttcatgattattgggggcaggggactcaggtcactgtatcctcaggagctggatccggaggtggaggctccggcaccatgatggatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtctccagcatgcatagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 311 - DNA, 3568 bp, synthetic construct
  source 1..3568: mol_type = other DNA
taatacgactcactatagggagacccaagctggctagaggatcgaacccttaaggccaccatggcgagcaattttacccagtttgtgcttgtggacaacggcggcaccggggacgtgacggtggccccctccaattttgccaatggcattgcagaatggataagctctaacagcaggagccaggcatacaaggtgacctgcagcgtgaggcagtcaagcgctcaaaacaggaagtacaccattaaggtcgaagtgcccaaaggagcttggaggtcttacctgaacatggaactgacaattcctatcttcgcgaccaatagcgactgtgagctgatcgtgaaggccatgcaaggcctgctgaaagacgggaatcccatacccagcgccatcgccgctaactcaggcatttacgctaatttcactcagttcgtactggttgacaatgggggaaccggcgacgttaccgtggctccaagcaacttcgctaacgggatcgccgagtggatcagcagtaattcacgctcccaagcctacaaagtaacctgctctgtacggcagagttcagcccagaaccgaaagtataccatcaaagtggaggtgccgaagggcgcctggcggagctatctgaatatggagctgaccatccccatctttgccacgaacagcgattgcgagctcatcgtcaaggcgatgcagggcttgctgaaggatggcaaccctatcccgagcgcaatagcagccaacagcggcatctatgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggatcaggtccaggacgcctggaggaagaacttcggaggagactttctcctggaaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgatgtggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaatactctctgacggggagcggaggtaccgccgaagttcaattacaggaatcgggtggaggtctggtacaacctgggggctctcttcgcctgagttgcactgccagtggagttacgatttctgcacttaatgctatggcgatgggttggtatcgtcaggccccaggggaacgtcgcgtcatggtcgctgccgtttccgaacgtggcaatgctatgtaccgcgagtctgttcagggccgcttcacggttacccgcgattttacaaataaaatggtatcgttgcaaatggacaacttaaagccagaggacactgctgtgtactactgtcacgtccttgaagatcgtgtggattcctttcatgattattgggggcaggggactcaggtcactgtatcctcaggagctggatccggaggtggaggctccggcaccatgatggatcaagtccaactggtggagtctggtggcgctttggtgcagccaggtggctctctgcgtttgtcctgtgccgcttctggcttcccagtgaaccgctattccatgcgctggtatcgccaggctccaggcaaagagcgtgagtgggtagccggtatgtccagcgcgggtgatcgtagctcctatgaagactccgtgaagggccgtttcaccatcagccgtgacgatgcccgtaacacggtgtatctgcaaatgaacagcttgaaacctgaagatacggccgtgtattactgtaatgtgaacgtgggcttcgagtattggggccaaggcacccaggtcaccgtctccagcatgcatagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 312 - DNA, 918 bp, synthetic construct
  source 1..918: mol_type = other DNA
agctcgtttagtgaaccgtcagatcgcctggagacgccatccacgctgttttgacctccatagaagacaccgggaccgatccagcctccggactctagcgtttaaacttaagcttgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggaaatgaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatcctaatctagagggccctattctatagtgtcacctaaatgctagagctcgctgatcagcctcgactgtgccttcta

SEQ ID NO: 313 - DNA, 954 bp, synthetic construct
  source 1..954: mol_type = other DNA
agctcgtttagtgaaccgtcagatcgcctggagacgccatccacgctgttttgacctccatagaagacaccgggaccgatccagcctccggactctagcgtttaaacttaagcttgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggaaatgaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatcccgcctggaggaagaacttcggaggagacttactgagtaatctagagggccctattctatagtgtcacctaaatgctagagctcgctgatcagcctcgactgtgccttcta

SEQ ID NO: 314 - DNA, 3538 bp, synthetic construct
  source 1..3538: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctaccggtgctccacccaatctctgggcagcgcagcgctacggccgtgagctcagaaggatgtccgatgagttcgtcgattccttcaaaaaggctagccagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggatcaggtccaggacgcctggaggaagaacttcggaggagactttctcctggaaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaatactctctgacggggagcggaggtaccgccgaagttcaattacaggaatcgggtggaggtctggtacaacctgggggctctcttcgcctgagttgcactgccagtggagttacgatttctgcacttaatgctatggcgatgggttggtatcgtcaggccccaggggaacgtcgcgtcatggtcgctgccgtttccgaacgtggcaatgctatgtaccgcgagtctgttcagggccgcttcacggttacccgcgattttacaaataaaatggtatcgttgcaaatggacaacttaaagccagaggacactgctgtgtactactgtcacgtccttgaagatcgtgtggattcctttcatgattattgggggcaggggactcaggtcactgtatcctcaggagctggatctggagcctcaagtaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 315 - DNA, 2845 bp, synthetic construct; identical to SEQ ID NO: 205
  source 1..2845: mol_type = other DNA

SEQ ID NO: 316 - DNA, 3484 bp, synthetic construct
  source 1..3484: mol_type = other DNA
taatacgactcactatagggagacccaagctggctagaggatcgaacccttaaggccaccatggcgagcaattttacccagtttgtgcttgtggacaacggcggcaccggggacgtgacggtggccccctccaattttgccaatggcattgcagaatggataagctctaacagcaggagccaggcatacaaggtgacctgcagcgtgaggcagtcaagcgctcaaaacaggaagtacaccattaaggtcgaagtgcccaaaggagcttggaggtcttacctgaacatggaactgacaattcctatcttcgcgaccaatagcgactgtgagctgatcgtgaaggccatgcaaggcctgctgaaagacgggaatcccatacccagcgccatcgccgctaactcaggcatttacgctaatttcactcagttcgtactggttgacaatgggggaaccggcgacgttaccgtggctccaagcaacttcgctaacgggatcgccgagtggatcagcagtaattcacgctcccaagcctacaaagtaacctgctctgtacggcagagttcagcccagaaccgaaagtataccatcaaagtggaggtgccgaagggcgcctggcggagctatctgaatatggagctgaccatccccatctttgccacgaacagcgattgcgagctcatcgtcaaggcgatgcagggcttgctgaaggatggcaaccctatcccgagcgcaatagcagccaacagcggcatctatgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcgtcgtccggtggagaacttgatgaattggtatacttactagatgggccaggttatgaccctatacattgcgatgtagtgacaaggggcggcagccaccttttcaattttgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacgggggggagcgcggccggaggtagcggcggaagcgcggccgctcaggggtctgttgttattgttggtagaattattttatctggtagtggtagtatcacggcctactcccaacagacgcggggcctacttggttgcatcatcactagcctcacaggccgggacaagaaccaggtcgaaggggaggttcaagtggtttctaccgcaacacaatctttcctggcgacctgcgtcaacggcgtgtgctggactgtctaccatggcgctggctcgaagaccctagccggtccaaaaggtccaatcacccaaatgtacaccaatgtagaccaggacctcgtcggctggcaggcgcctccaggggcgcgctccttgacaccatgcacctgtggcagctcggacctttacttggtcacgagacatgctgatgtcattccggtgcgccggcgaggcgacagcaggggaagtctactctcccccaggcccgtctcctacctgaaaggctcctcaggtggtccattgctttgcccttcggggcacgctgtgggcatcttccgggctgctgtgtgcacccggggggtcgcgaaggcggtggacttcgtgcccgttgagtctatggaaactaccatgcggtctgagagtggatcaggtaccatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 317 - DNA, 3484 bp, synthetic construct
  source 1..3484: mol_type = other DNA
taatacgactcactatagggagacccaagctggctagaggatcgaacccttaaggccaccatggcgagcaattttacccagtttgtgcttgtggacaacggcggcaccggggacgtgacggtggccccctccaattttgccaatggcattgcagaatggataagctctaacagcaggagccaggcatacaaggtgacctgcagcgtgaggcagtcaagcgctcaaaacaggaagtacaccattaaggtcgaagtgcccaaaggagcttggaggtcttacctgaacatggaactgacaattcctatcttcgcgaccaatagcgactgtgagctgatcgtgaaggccatgcaaggcctgctgaaagacgggaatcccatacccagcgccatcgccgctaactcaggcatttacgctaatttcactcagttcgtactggttgacaatgggggaaccggcgacgttaccgtggctccaagcaacttcgctaacgggatcgccgagtggatcagcagtaattcacgctcccaagcctacaaagtaacctgctctgtacggcagagttcagcccagaaccgaaagtataccatcaaagtggaggtgccgaagggcgcctggcggagctatctgaatatggagctgaccatccccatctttgccacgaacagcgattgcgagctcatcgtcaaggcgatgcagggcttgctgaaggatggcaaccctatcccgagcgcaatagcagccaacagcggcatctatgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcgtcgtccggtggagaacttgatgaattggtatacttactagatgggccaggttatgaccctatacattgcgatgtagtgacaaggggcggcagccaccttttcaattttgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaggccgaccgagcaggaccaattctctgtgacgggggggagcgcggccggaggtagcggcggaagcgcggccgctcaggggtctgttgttattgttggtagaattattttatctggtagtggtagtatcacggcctactcccaacagacgcggggcctacttggttgcatcatcactagcctcacaggccgggacaagaaccaggtcgaaggggaggttcaagtggtttctaccgcaacacaatctttcctggcgacctgcgtcaacggcgtgtgctggactgtctaccatggcgctggctcgaagaccctagccggtccaaaaggtccaatcacccaaatgtacaccaatgtagaccaggacctcgtcggctggcaggcgcctccaggggcgcgctccttgacaccatgcacctgtggcagctcggacctttacttggtcacgagacatgctgatgtcattccggtgcgccggcgaggcgacagcaggggaagtctactctcccccaggcccgtctcctacctgaaaggctcctcaggtggtccattgctttgcccttcggggcacgctgtgggcatcttccgggctgctgtgtgcacccggggggtcgcgaaggcggtggacttcgtgcccgttgagtctatggaaactaccatgcggtctgagagtggatcaggtaccatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 318 - DNA, 3037 bp, synthetic construct
  source 1..3037: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggagaagatgttgtctgctgtcattcaatctacggcaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggtaccatgaaaaagaaaggttctgttgttattgttggtagaattaatttatctggtgacacggcctactcccaacagacgcggggcctagaaggttgccaagagactagccaaacaggccgggacaagaaccaggtcgaaggggaggttcaagtggtttctaccgcaacacaatctttcctggcgacctccatcaacggcgtgctttggactgtctaccatggcgctggcaccagaaccattgccagcccaaaaggtccagtgacccaaatgtacaccaatgtagacaaggacctcgtcggctggcaggcgcctcaagggtcacgctccttgacaccatgcacctgtggcagctcggacctttacttggtcacgagacatgctgatgtcattccggtgcgccggcgaggcgacagcaggggaagtctactctcccccaggcccatctcctacctgaaaggctcctcaggtggtccattgctttgccctgctgggcacgctgtgggcatcttccgggctgctgtgagtacccggggggtcgcgaaggcggtggacttcattcccgttgagtctctggaaactaccatgcggtctccaggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 319 - DNA, 3037 bp, synthetic construct
  source 1..3037: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggagaagatgttgtctgctgtcattcaatctacggcaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggtaccatgaaaaagaaaggttctgttgttattgttggtagaattaatttatctggtgacacggcctactcccaacagacgcggggcctagaaggttgccaagagactagccaaacaggccgggacaagaaccaggtcgaaggggaggttcaagtggtttctaccgcaacacaatctttcctggcgacctccatcaacggcgtgctttggactgtctaccatggcgctggcaccagaaccattgccagcccaaaaggtccagtgacccaaatgtacaccaatgtagacaaggacctcgtcggctggcaggcgcctcaagggtcacgctccttgacaccatgcacctgtggcagctcggacctttacttggtcacgagacatgctgatgtcattccggtgcgccggcgaggcgacagcaggggaagtctactctcccccaggcccatctcctacctgaaaggctccgcaggtggtccattgctttgccctgctgggcacgctgtgggcatcttccgggctgctgtgagtacccggggggtcgcgaaggcggtggacttcattcccgttgagtctctggaaactaccatgcggtctccaggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 320 - DNA, 3037 bp, synthetic construct
  source 1..3037: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggagaagatgttgtctgctgtcattcaatctacggcaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctgggacggggagcggtaccatgaaaaagaaaggttctgttgttattgttggtagaattaatttatctggtgacacggcctactcccaacagacgcggggcctagaaggttgccaagagactagccaaacaggccgggacaagaaccaggtcgaaggggaggttcaagtggtttctaccgcaacacaatctttcctggcgacctccatcaacggcgtgctttggactgtctaccatggcgctggcaccagaaccattgccagcccaaaaggtccagtgacccaaatgtacaccaatgtagacaaggacctcgtcggctggcaggcgcctcaagggtcacgctccttgacaccatgcacctgtggcagctcggacctttacttggtcacgagacatgctgatgtcattccggtgcgccggcgaggcgacagcaggggaagtctactctcccccaggcccatctcctacctgaaaggctcctcaggtggtccattgctttgccctgctgggcacgctgtgggcatcttccgggctgctgtgagtacccggggggtcgcgaaggcggtggacttcattcccgttgagtctctggaaactaccatgcggtctccaggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 321 - DNA, 3037 bp, synthetic construct
  source 1..3037: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggagaagatgttgtctgctgtcattcaatctacggcaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctgggacggggagcggtaccatgaaaaagaaaggttctgttgttattgttggtagaattaatttatctggtgacacggcctactcccaacagacgcggggcctagaaggttgccaagagactagccaaacaggccgggacaagaaccaggtcgaaggggaggttcaagtggtttctaccgcaacacaatctttcctggcgacctccatcaacggcgtgctttggactgtctaccatggcgctggcaccagaaccattgccagcccaaaaggtccagtgacccaaatgtacaccaatgtagacaaggacctcgtcggctggcaggcgcctcaagggtcacgctccttgacaccatgcacctgtggcagctcggacctttacttggtcacgagacatgctgatgtcattccggtgcgccggcgaggcgacagcaggggaagtctactctcccccaggcccatctcctacctgaaaggctccgcaggtggtccattgctttgccctgctgggcacgctgtgggcatcttccgggctgctgtgagtacccggggggtcgcgaaggcggtggacttcattcccgttgagtctctggaaactaccatgcggtctccaggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 322 - DNA, 3037 bp, synthetic construct
  source 1..3037: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggagaagatgttgtctgctgtcattcaatctacggcaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaatactctctgacggggagcggtaccatgaaaaagaaaggttctgttgttattgttggtagaattaatttatctggtgacacggcctactcccaacagacgcggggcctagaaggttgccaagagactagccaaacaggccgggacaagaaccaggtcgaaggggaggttcaagtggtttctaccgcaacacaatctttcctggcgacctccatcaacggcgtgctttggactgtctaccatggcgctggcaccagaaccattgccagcccaaaaggtccagtgacccaaatgtacaccaatgtagacaaggacctcgtcggctggcaggcgcctcaagggtcacgctccttgacaccatgcacctgtggcagctcggacctttacttggtcacgagacatgctgatgtcattccggtgcgccggcgaggcgacagcaggggaagtctactctcccccaggcccatctcctacctgaaaggctcctcaggtggtccattgctttgccctgctgggcacgctgtgggcatcttccgggctgctgtgagtacccggggggtcgcgaaggcggtggacttcattcccgttgagtctctggaaactaccatgcggtctccaggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 323 - DNA, 3037 bp, synthetic construct
  source 1..3037: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcggagaagatgttgtctgctgtcattcaatctacggcaccggtgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaatactctctgacggggagcggtaccatgaaaaagaaaggttctgttgttattgttggtagaattaatttatctggtgacacggcctactcccaacagacgcggggcctagaaggttgccaagagactagccaaacaggccgggacaagaaccaggtcgaaggggaggttcaagtggtttctaccgcaacacaatctttcctggcgacctccatcaacggcgtgctttggactgtctaccatggcgctggcaccagaaccattgccagcccaaaaggtccagtgacccaaatgtacaccaatgtagacaaggacctcgtcggctggcaggcgcctcaagggtcacgctccttgacaccatgcacctgtggcagctcggacctttacttggtcacgagacatgctgatgtcattccggtgcgccggcgaggcgacagcaggggaagtctactctcccccaggcccatctcctacctgaaaggctccgcaggtggtccattgctttgccctgctgggcacgctgtgggcatcttccgggctgctgtgagtacccggggggtcgcgaaggcggtggacttcattcccgttgagtctctggaaactaccatgcggtctccaggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 324 - DNA, 5421 bp, synthetic construct
  source 1..5421: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcgaaacatgaggatcacccatgtgccgctatggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttctggcaccggtatggcatctatgactggaggccaacagatgggtcctgcaaccgggaattccgcgtagcgctagctttgccagcgccacgcgaaacatgaggatcacccatgtactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctatcgatgtgagcaagggcgaagaagataacaaggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctctagagtcgacgggccgcggtaacaattgttatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcgggtcgggcaccggtgctccacccaatctctgggcagcgcagcgctacggccgtgagctcagaaggatgtccgatgagttcgtcgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgatgtggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggaggtacggagaatttgtattttcagagcggctcgagtggtggatcagggagcgccgcttcaggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 325 - DNA, 5421 bp, synthetic construct
  source 1..5421: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcgaaacatgaggatcacccatgtgccgctatggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttctggcaccggtatggcatctatgactggaggccaacagatgggtcctgcaaccgggaattccgcgtagcgctagctttgccagcgccacgcgaaacatgaggatcacccatgtactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctatcgatgtgagcaagggcgaagaagataacaaggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctctagagtcgacgggccgcggtaacaattgttatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcgggtcgggcaccggtgctccacccaatctctgggcagcgcagcgctacggccgtgagctcagaaggatgtccgatgagctggtcgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgatgtggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggaggtacggagaatttgtattttcagagcggctcgagtggtggatcagggagcgccgcttcaggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 326 - DNA, 5421 bp, synthetic construct
  source 1..5421: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcgaaacatgaggatcacccatgtgccgctatggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttctggcaccggtatggcatctatgactggaggccaacagatgggtcctgcaaccgggaattccgcgtagcgctagctttgccagcgccacgcgaaacatgaggatcacccatgtactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctatcgatgtgagcaagggcgaagaagataacaaggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctctagagtcgacgggccgcggtaacaattgttatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcgggtcgggcaccggtgctccacccaatctctgggcagcgcagcgctacggccgtgagctcagaaggatgtccgatgaggtggtcgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgatgtggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggaggtacggagaatttgtattttcagagcggctcgagtggtggatcagggagcgccgcttcaggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 327 - DNA, 5748 bp, synthetic construct
  source 1..5748: mol_type = other DNA
taatacgactcactatagggagacccaagctggctaggtaagcttggtaccgagctcggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcgaaacatgaggatcacccatgtgccgctatggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttctggcaccggtatggcatctatgactggaggccaacagatgggtcctgcaaccgggaattccgcgtagcgctagctttgccagcgccacgcgaaacatgaggatcacccatgtactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctatcgatgtgagcaagggcgaagaagataacaaggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctctagagtcgacgggccgcggtaacaattgttatggcgagcaattttacccagtttgtgcttgtggacaacggcggcaccggggacgtgacggtggccccctccaattttgccaatggcattgcagaatggataagctctaacagcaggagccaggcatacaaggtgacctgcagcgtgaggcagtcaagcgctcaaaacaggaagtacaccattaaggtcgaagtgcccaaaggagcttggaggtcttacctgaacatggaactgacaattcctatcttcgcgaccaatagcgactgtgagctgatcgtgaaggccatgcaaggcctgctgaaagacgggaatcccatacccagcgccatcgccgctaactcaggcatttacgctaatttcactcagttcgtactggttgacaatgggggaaccggcgacgttaccgtggctccaagcaacttcgctaacgggatcgccgagtggatcagcagtaattcacgctcccaagcctacaaagtaacctgctctgtacggcagagttcagcccagaaccgaaagtataccatcaaagtggaggtgccgaagggcgcctggcggagctatctgaatatggagctgaccatccccatctttgccacgaacagcgattgcgagctcatcgtcaaggcgatgcagggcttgctgaaggatggcaaccctatcccgagcgcaatagcagccaacagcggcatctatgggggcagtgggagcggtgcaggatctggtagtccagctgggggaggagcaccgggtagcggtggggggtctaccggtgctccacccaatctctgggcagcgcagcgctacggccgtgagctcagaaggatgtccgatgagttcgtcgattccttcaaaaaggctagccagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaatactctctgacggggagcggaggtacggagaatttgtattttcagagcgccgcttcaggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaagggcccgtttaaacccgctgatcagcctcgactgtgccttcta

SEQ ID NO: 328 - protein, 334 aa, synthetic construct
METDTLLLWVLLLWVPGSTGDGGGGYPYDVPDYAGELDELVYLLDGPGYDPIHSGGSGGRTMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYQLENGGISLLVQNTSWMLLLLLSLSLLQALDFISL

SEQ ID NO: 329 - protein, 315 aa, synthetic construct
IETDTLLLWVLLLWVPGSTGDGGGGYPYDVPDYAGGGSGGRTMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYQLENGGISLLVQNTSWMLLLLLSLSLLQALDFISL

SEQ ID NO: 330 - protein, 640 aa, synthetic construct
MALPVTALLLPLALLLHAARPYPYDVPDYATGMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGSGEGRGSLLTCGDVEENPRPYMRITHVRPRVDYKDHDGDYKDHDIDYKDDDDKGTMEGRGSLLTCGDVEENPGPGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 331 - protein, 296 aa, synthetic construct; identical to SEQ ID NO: 1

SEQ ID NO: 332 - protein, 233 aa, synthetic construct
LCQRHAKHEDHPCTSATNFSLLKQAGDVEENPGPGGSEGRGSLLTCGDVEENPGPSGYPYDVPDYAHMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFK

SEQ ID NO: 333 - protein, 81 aa, synthetic construct
SYTTGVAPLDTMRITHGKRYRSTARTTYTFAKPMAANYLKNQPMYVFRKTELKHSKTELNFKEWQKAFTDVMGMDELYKAS

SEQ ID NO: 334 - protein, 1155 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYANFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASPSRLEEELRRRLTEPTGSGAGDRHPNRKARGQLRTKIESGQGTIPVRSNASIQTWDGVLQGERLLTMSCSDKIAMWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSVTGSGGTAEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSGAGSGGGGSGTMMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSMHSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 335 - protein, 1155 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYANFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASPSRLEEELRRRLTEPTGSGAGDRHPNRKARGQLRTKIESGQGTIPVRSNASIQTWDGVLQGERLLTMSCSDKIAMWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSGTGSGGTAEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSGAGSGGGGSGTMMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSMHSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 336 - protein, 1155 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYANFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASPSRLEEELRRRLTEPTGSGAGDRHPNRKARGQLRTKIESGQGTIPVRSNASIQTWDGVLQGERLLTMSCSDKIAMWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQYSLTGSGGTAEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSGAGSGGGGSGTMMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSMHSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 337 - protein, 1154 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYANFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGPGRLEEELRRRLSPGTGDRHPNRKARGQLRTKIESGQGTIPVRSNASIQTWDGVLQGERLLTMSCSDKIAMWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSVTGSGGTAEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSGAGSGGGGSGTMMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSMHSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 338 - protein, 1154 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYANFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGPGRLEEELRRRLSPGTGDRHPNRKARGQLRTKIESGQGTIPVRSNASIQTWDGVLQGERLLTMSCSDKIAMWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSGTGSGGTAEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSGAGSGGGGSGTMMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSMHSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 339 - protein, 1154 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYANFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGPGRLEEELRRRLSPGTGDRHPNRKARGQLRTKIESGQGTIPVRSNASIQTWDGVLQGERLLTMSCSDKIAMWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQYSLTGSGGTAEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSGAGSGGGGSGTMMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSMHSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 340 - protein, 241 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQEMTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGS

SEQ ID NO: 341 - protein, 253 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQEMTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSRLEEELRRRLTE

SEQ ID NO: 342 - protein, 1144 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSTGAPPNLWAAQRYGRELRRMSDEFVDSFKKASQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGPGRLEEELRRRLSPGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQYSLTGSGGTAEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSGAGSGASSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 343 - protein, 913 aa, synthetic construct; identical to SEQ ID NO: 168

SEQ ID NO: 344 - protein, 1125 aa, synthetic construct; identical to SEQ ID NO: 169

SEQ ID NO: 345 - protein, 1125 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYANFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPASSGGELDELVYLLDGPGYDPIHCDVVTRGGSHLFNFDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVERPTEQDQFSVTGSAAGGSGGSAAAQGSVVIVGRIILSGSGSITAYSQQTRGLLGCIITSLTGRDKNQVEGEVQVVSTATQSFLATCVNGVCWTVYHGAGSKTLAGPKGPITQMYTNVDQDLVGWQAPPGARSLTPCTCGSSDLYLVTRHADVIPVRRRGDSRGSLLSPRPVSYLKGSSGGPLLCPSGHAVGIFRAAVCTRGVAKAVDFVPVESMETTMRSESGSGTMSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 346 - protein, 977 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGEDVVCCHSIYGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGTMKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 347 - protein, 977 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGEDVVCCHSIYGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGTMKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 348 - protein, 977 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGEDVVCCHSIYGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSGTGSGTMKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 349 - protein, 977 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGEDVVCCHSIYGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSGTGSGTMKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 350 - protein, 977 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGEDVVCCHSIYGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQYSLTGSGTMKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSSGGPLLCPAGHAVGIFRAAVSTRGVAKAVDFIPVESLETTMRSPGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 351 - protein, 977 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGEDVVCCHSIYGTGDRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQYSLTGSGTMKKKGSVVIVGRINLSGDTAYSQQTRGLEGCQETSQTGRDKNQVEGEVQVVSTATQSFLATSINGVLWTVYHGAGTRTIASPKGPVTQMYTNVDKDLVGWQAPQGSRSLTPCTCGSSDLYLVTRHADVIPVRRRGDSRGSLLSPRPISYLKGSAGGPLLCPAGHAVGIFRAAVSTRGVAKAVDFIPVESLETTMRSPGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 352 - protein, 812 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDEFVDRHPNRKARGQLRTKIESGQGTIPVRSNASIQTWDGVLQGERLLTMSCSDKIAMWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGGTENLYFQSGSSGGSGSAAGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 353 - protein, 812 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDELVDRHPNRKARGQLRTKIESGQGTIPVRSNASIQTWDGVLQGERLLTMSCSDKIAMWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGGTENLYFQSGSSGGSGSAAGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 354 - protein, 812 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPAASGSGTGAPPNLWAAQRYGRELRRMSDEVVDRHPNRKARGQLRTKIESGQGTIPVRSNASIQTWDGVLQGERLLTMSCSDKIAMWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTGSGGTENLYFQSGSSGGSGSAAGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 355 - protein, 922 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYANFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGSGSGAGSGSPAGGGAPGSGGGSTGAPPNLWAAQRYGRELRRMSDEFVDSFKKASQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQYSLTGSGGTENLYFQSAASGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 356 - DNA, 2032 bp, synthetic construct; identical to SEQ ID NO: 301
  source 1..2032: mol_type = other DNA

SEQ ID NO: 357 - DNA, 250 bp, synthetic construct
  source 1..250: mol_type = other DNA
taatacgactcactatagggagacccaagcttggtaccactagtgccacaaacttctctctgctaaagcaagcaggtgatgttgaagaaaacccagggcctggagggtccgagggcaggggaagtctcctaacatgcggggacgtggaggaaaatcccggaccagaattcgccacctgatctagagggccctattctatagtgtcacctaaatgctagagctcgctgatcagcctcgactgtgccttcta

SEQ ID NO: 358 - protein, 640 aa, synthetic construct; identical to SEQ ID NO: 330

SEQ ID NO: 359 - protein, 46 aa, synthetic construct
TSATNFSLLKQAGDVEENPGPGGSEGRGSLLTCGDVEENPGPEFAT

SEQ ID NO: 360 - protein, 19 aa, unidentified
ATNFSLLKQAGDVEENPGP

SEQ ID NO: 361 - protein, 18 aa, Thosea asigna virus
EGRGSLLTCGDVEENPGP

SEQ ID NO: 362 - protein, 20 aa, Equine rhinitis A virus
QCTNYALLKLAGDVESNPGP

SEQ ID NO: 363 - protein, 22 aa, unidentified
VKQTLNFDLLKLAGDVESNPGP

SEQ ID NO: 364 - protein, 10 aa, synthetic construct
EDVVCCHSIY

SEQ ID NO: 365 - protein, 14 aa, synthetic construct
LYQEFDEMEECSQH

SEQ ID NO: 366 - protein, 10 aa, synthetic construct
DEMEECSQHL

SEQ ID NO: 367 - protein, 13 aa, synthetic construct
QEFEDVVPCSMGS

SEQ ID NO: 368 - protein, 9 aa, synthetic construct
EDVVCCHSI

SEQ ID NO: 369 - protein, 9 aa, synthetic construct
DEMEECSQH

SEQ ID NO: 370 - protein, 20 aa, synthetic construct
CMSADLEVVTSTWVLVGGVL

SEQ ID NO: 371 - protein, 20 aa, synthetic construct
CMSADLEVVTSTWVLVGGVL

SEQ ID NO: 372 - protein, 20 aa, synthetic construct
CMQADLEVMTSTWVLAGGVL

SEQ ID NO: 373 - protein, 20 aa, synthetic construct
CMQADLEIMTSSWVLAGGVL

SEQ ID NO: 374 - protein, 20 aa, synthetic construct
CMSADLEVTTSTWVLLGGVL

SEQ ID NO: 375 - protein, 20 aa, synthetic construct
YQEFDEMEECSQHLPYIEQG

SEQ ID NO: 376 - protein, 20 aa, synthetic construct
YQEFDEMEECASHLPYIEQG

SEQ ID NO: 377 - protein, 20 aa, synthetic construct
YEAFDEMEECASRAALIEEG

SEQ ID NO: 378 - protein, 20 aa, synthetic construct
YEAFDEMEECASKAALIEEG

SEQ ID NO: 379 - protein, 20 aa, synthetic construct
YQQYDEMEECSQAAPYIEQA

SEQ ID NO: 380 - protein, 20 aa, synthetic construct
WISSECTTPCSGSWLRDVWD

SEQ ID NO: 381 - protein, 20 aa, synthetic construct
WINEDCSTPCSGSWLRDVWD

SEQ ID NO: 382 - protein, 20 aa, synthetic construct
WITEDCPIPCSGSWLRDVWD

SEQ ID NO: 383 - protein, 20 aa, synthetic construct
WITEDCPVPCSGSWLQDIWD

SEQ ID NO: 384 - protein, 20 aa, synthetic construct
WINEDYPSPCSDDWLRTIWD

SEQ ID NO: 385 - protein, 20 aa, synthetic construct
GADTEDVVCCSMSYSWTGAL

SEQ ID NO: 386 - protein, 20 aa, synthetic construct
EEASEDVVCCSMSYTWTGAL

SEQ ID NO: 387 - protein, 20 aa, synthetic construct
SEEDDSVVCCSMSYSWTGAL

SEQ ID NO: 388 - protein, 20 aa, synthetic construct
SDQEDSVICCSMSYSWTGAL

SEQ ID NO: 389 - protein, 20 aa, synthetic construct
DSEEQSVVCCSMSYSWTGAL

SEQ ID NO: 390 - DNA, 4665 bp, synthetic construct
  source 1..4665: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgccataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagcgcccatcggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcgaaacatgaggatcacccatgtgccgctatggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttctggcaccggtatggcatctatgactggaggccaacagatgggtcctgcaaccgggaattccgcgtagcgctagctttgccagcgccacgcgaaacatgaggatcacccatgtactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctatcgatgtgagcaagggcgaagaagataacaaggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttcta

SEQ ID NO: 391 - DNA, 4665 bp, synthetic construct
  source 1..4665: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggatccagcgagctgattaaggagaacatgcacatgaagcgcccatcggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcgaaacatgaggatcacccatgtgccgctatggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttctggcaccggtatggcatctatgactggaggccaacagatgggtcctgcaaccgggaattccgcgtagcgctagctttgccagcgccacgcgaaacatgaggatcacccatgtactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctatcgatgtgagcaagggcgaagaagataacaaggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttcta

SEQ ID NO: 392 - DNA, 5376 bp, synthetic construct
  source 1..5376: mol_type = other DNA
taatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcgggtcgggcaccggtgctccacccaatctctgggcagcgcagcgctacggccgtgagctcagaaggatgtccgatgaggtggtcgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccagggctcgactttttacagcttttattaaggcagggctcggggcatgggtcgagaagccgaccgagcaggaccaattctctctgacggggagcgcggccggaggtagcggcggaagcgcggccgcctcaagtaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatggatccagcgagctgattaaggagaacatgcacatgaagcgcccatcggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcgaaacatgaggatcacccatgtgccgctatggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttctggcaccggtatggcatctatgactggaggccaacagatgggtcctgcaaccgggaattccgcgtagcgctagctttgccagcgccacgcgaaacatgaggatcacccatgtactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctatcgatgtgagcaagggcgaagaagataacaaggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttcta

SEQ ID NO: 393 - protein, 332 aa, synthetic construct
LCQRHAKHEDHPCAAMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGTGMASMTGGQQMGPATGNSA

SEQ ID NO: 394 - protein, 15 aa, synthetic construct
LCQRHAKHEDHPCTS

SEQ ID NO: 395 - RNA, 66 nt, synthetic construct
  source 1..66: mol_type = other RNA
aagaattccgcgtagcgctagctttgccagcgccacgcgaaacatgaggatcacccatgtactagt

SEQ ID NO: 396 - RNA, 66 nt, synthetic construct
  source 1..66: mol_type = other RNA
aagaattccgcgtagcgctggctttgccagcgccacgcgaaacatgaggatcacccatgtactagt

SEQ ID NO: 397 - RNA, 66 nt, synthetic construct
  source 1..66: mol_type = other RNA
aagaattccgcgtggcgctagctttgccagcgccacgcgaaacatgaggatcacccatgtactagt

SEQ ID NO: 398 - RNA, 63 nt, synthetic construct
  source 1..63: mol_type = other RNA
aagaattccgcgtagcgctagctacatgaggatcacccatgttgccagcgccacgcgactagt

SEQ ID NO: 399 - RNA, 75 nt, synthetic construct
  source 1..75: mol_type = other RNA
aagaattccgcgtagcgctagctttgccagcgccacgcgaaggagcagacgatatggcgtcgctccaatactagt

SEQ ID NO: 400 - RNA, 78 nt, synthetic construct
  source 1..78: mol_type = other RNA
aagaattccgcgtagcgctagctttgccagcgccacgcggtaggctcgtctgagctcattagctccgagccaactagt

SEQ ID NO: 401 - RNA, 69 nt, synthetic construct
  source 1..69: mol_type = other RNA
aagaattccgcgtagcgctagctttgccagcgccacgcggtaagggccctgaagaagggcccaactagt

SEQ ID NO: 402 - DNA, 4367 bp, synthetic construct
  source 1..4367: mol_type = other DNA
cttatcgaaattaatacgactcactatagggagacccaagctggctagaggatcgaacccttaaggccaccatggcgtccaatttcactcagtttgtgctggttgacaacggcgggaccggggacgttacggtagccccctcaaactttgccaacggtatagcggagtggataagcagcaattctaggagtcaagcatacaaagttacatgcagcgtgcgccaatctagcgctcagaatcgcaagtacaccattaaagtagaggtccccaagggagcctggagaagctatcttaacatggagttgaccataccaatcttcgctaccaactctgactgtgaactcattgtgaaagccatgcaaggtctgctcaaggatggtaacccaattccgtccgctatcgctgccaactctgggatttacgggggcagtgggagcggtgcaggatctggtagtccagctgggggaggagcaccgggtagcggtggggggtctcagctgcacctgccccaggttctcgcagacgccgtatcccgccttgtactgggcaagtttggtgatcttactgacaatttttcatctcctcatgcgaggcggaaagtactcgcaggcgtcgtcatgacgaccggaactgacgtgaaagacgccaaagtcatctctgtctccacgggcacaaagtgcataaacggggagtacatgagcgaccgggggctggcactgaatgattgtcacgctgaaataatatctaggcgatctctgcttagatttctctacactcaactcgaattgtaccttaacaacaaagatgaccagaaacgcagtatatttcagaaatcagaacgcggcggatttcgacttaaggaaaacgttcagttccacttgtatatcagcacatccccttgcggtgacgcccgaatcttttccccgcacgagccgatattggaggagcccgcggctagcgggtcgggcaccggtgctccacccaatctctgggcagcgcagcgctacggccgtgagctcagaaggatgtccgatgagttcgtcgacagacatcctaataggaaggctagaggccaacttcggacgaagattgaaagtggccagggtactatcccggtgcggtccaacgctagtattcaaacgtgggacggagtccttcaaggtgaacggctgttgacaatgagctgctcagacaaaatcgcgcgctggaatgtagtgggaatccaaggcagcctcttgagcatattcgtagaacccatatatttctcatccattattttgggctctctgtatcatggtgaccatctgtcaagggctatgtaccaacgaatttctaatatcgaggatcttcctccactctatacactcaataagcctctcttgtccgggatatcaaacgctgaggcccgccagccagggaaagctcctaacttcagtgttaactggaccgttggtgattctgcgatagaggtcatcaacgccacgacaggtaaggatgagctcggtagagcctcacgcctgtgtaaacacgcgttgtattgtagatggatgagagtacatgggaaggtcccatctcacttgctccgaagcaagatcactaagcctaatgtgtatcatgagtcaaaactcgcggctaaagaataccaggcagccaaagctcgactttttacagcttttattaaggcagggctcggggcatgggtcgagaagccgaccgagcaggaccaattctctctgacggggagcggaggtacggagaatttgtattttcagagcgccgcttcaagtaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatggatccagcgagctgattaaggagaacatgcacatgaagcgcccatcggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggattacaaggatgacgatgacaaaggtagcggggcaactaattttagcttactcaaacaggctggggacgtcgaggagaatccaggccctgcatccgctggctctggagaaggacgaggctccttgctcacctgtggagatgtcgaagagaacccaggtcctgcaaccgggaattccgcgtagcgctagctttgccagcgccacgcgaaacatgaggatcacccatgtactagtgccacaaacttctctctgctaaagcaagcaggtgatgttgaagaaaacccagggcctggagggtccgagggcaggggaagtctcctaacatgcggggacgtggaggaaaatcccggcccatccggatatccctacgatgtgcccgattacgctatcgatgtgagcaagggcgaagaagataacaaggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggcagcggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagagataccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggaatggacgagctgtataaggctagctaagcggccgctcgagtctagagggcccgcggttcgaaggtaagcctatccctaaccctctcctcggtctcgattctacgcgtaccggtcatcatcaccatcaccattgagtttaaacccgctgatcag

SEQ ID NO: 403 - DNA, 19 bp, synthetic construct
  source 1..19: mol_type = other DNA
acatgaggatcacccatgt

SEQ ID NO: 404 - protein, 6 aa, synthetic construct
DDIVPC

SEQ ID NO: 405 - protein, 6 aa, synthetic construct
  SITE 1: note = Acetylated residue
DEMEEC

SEQ ID NO: 406 - protein, 8 aa, unidentified
DYKDDDDK

SEQ ID NO: 407 - RNA, 11 nt, synthetic construct
  source 1..11: mol_type = other RNA
aaaaaaaaaaa

SEQ ID NO: 408 - RNA, 27 nt, synthetic construct
  source 1..27: mol_type = other RNA
  misc_difference 1: note = a, c, u, g, unknown or other
  misc_difference 2: note = a, c, u, g, unknown or other
  misc_difference 6: note = a, c, u, g, unknown or other
  misc_difference 7: note = a, c, u, g, unknown or other
  misc_difference 8: note = a, c, u, g, unknown or other
  misc_difference 12: note = a, c, u, g, unknown or other
  misc_difference 13: note = a, c, u, g, unknown or other
  misc_difference 14: note = a, c, u, g, unknown or other
  misc_difference 15: note = a, c, u, g, unknown or other
  misc_difference 16: note = a, c, u, g, unknown or other
  misc_difference 20: note = a, c, u, g, unknown or other
  misc_difference 21: note = a, c, u, g, unknown or other
  misc_difference 22: note = a, c, u, g, unknown or other
  misc_difference 26: note = a, c, u, g, unknown or other
  misc_difference 27: note = a, c, u, g, unknown or other
nntagnnntagnnnnnccannnccann

SEQ ID NO: 409 - RNA, 46 nt, synthetic construct
  source 1..46: mol_type = other RNA
ctcaccatcgtcccgctgaggatcacccagcggccacgatagtgag

SEQ ID NO: 410 - RNA, 46 nt, synthetic construct
  source 1..46: mol_type = other RNA
ctcaccaccgtcccgctgaggatcacccagcggccacgatggtgag

SEQ ID NO: 411 - protein, 305 aa, synthetic construct
LCQRHAKHEDHPCTSATNFSLLKQAGDVEENPGPGGSEGRGSLLTCGDVEENPGPSGYPYDVPDYAIDVSKGEEDNKASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKAS

(19) United States
(12) Patent Application Publication
(10) Pub. No.: US 2024/0263193 A1
(43) Pub. Date: Aug. 8, 2024

(54) COMPOSITIONS AND METHODS FOR CONTROLLED MRNA TRANSLATION AND STABILITY

(71) Applicant: TRUSTEES OF BOSTON UNIVERSITY, Boston, MA (US)

(72) Inventors: Alexander Michael Marzilli, Cambridge, MA (US); John Tuan Ngo, Cambridge, MA (US)

(73) Assignee: TRUSTEES OF BOSTON UNIVERSITY, Boston, MA (US)

(21) Appl. No.: 18/392,928

(22) Filed: Dec. 21, 2023

Related U.S. Application Data
(60) Provisional application No. 63/434,275, filed on Dec. 21, 2022.

(51) Int. Cl. C12N 15/87; C07K 14/005; C12N 9/78; C12N 15/11

(52) U.S. Cl. CPC C12N 15/87; C07K 14/005; C12N 9/78; C12N 15/11; C12Y 305/04; C12N 2310/20; C12N 2310/531; C12N 2795/18022

(57) ABSTRACT

The technology described herein is directed to compositions, kits, systems and methods related to an engineered, inducible adenosine deaminase (iAD) enzymes, including but not limited to, an engineered inducible adenosine deaminase acting on RNA (ADAR) enzyme, which can be activated in the presence of an inducer. Also described are synthetic RNA molecules, to which the iAD can be specifically recruited to edit at least one target codon, leading to decreased or increased translation of the RNA molecules depending on the specific construct. The technology described herein is also directed to systems comprising the iAD and synthetic RNA molecule, nucleic acids and vectors encoding the iAD and synthetic RNA molecule, and methods of using such systems, nucleic acids, and vectors.

### CROSS-REFERENCE TO RELATED APPLICATIONS

This application claims benefit under 35 U.S.C. § 119(e) of U.S. Provisional Application No. 63/434,275 filed Dec. 21, 2022, the contents of which are incorporated herein by reference in their entirety.

### GOVERNMENT SUPPORT

This invention was made with government support under contract No. R35-GM128859 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Mar. 22, 2024, is named 701586-000107USPT_SL.xml and is 718,305 bytes in size.

### TECHNICAL FIELD

The technology described herein relates to methods and compositions for editing RNAs using an engineered inducible Adenosine Deaminase enzymes, including an Adenosine Deaminase Acting on RNA (iADAR) enzyme that is capable, in the presence of an inducer, to deaminate one or more adenosines in target RNAs, where the target RNA comprises a target codon, to regulate gene expression of a gene of interest.

### BACKGROUND

Nucleic acid editing carries enormous potential for biological research and the development of therapeutics. Current tools for DNA or RNA editing rely on introducing exogenous proteins into living organisms, which is subject to potential risks or technical barriers due to possible aberrant effector activity, delivery limits and immunogenicity. Moreover, nucleic acid based medicines, including messenger RNA (mRNA) based vaccines and therapeutics have rapidly developed in the past several years and have emerged as a promising technology with many potential applications in both medicine and basic science research. Instead of producing and delivering a protein directly to cells/organisms/patients, nucleic acids (including mRNAs) are delivered to cells via lipid nanoparticles (LNP) or other agents. Upon entry, ribosome mediated-translation results in the production of proteins encoded by the delivered nucleic acid sequences. A limitation of mRNA-based agents is that uptake of the mRNA to any human cell type will result in its translation and thus expression of the encoded protein. Thus, a limitation of mRNA based medicines is the limited control over translation of an encoded protein sequence.

Genome editing is a powerful tool for biomedical research and development of therapeutics for diseases. Editing technologies using engineered nucleases, such as zinc finger nucleases (ZFNs), transcription activator-like effector nucleases (TALENs), and Cas proteins of CRISPR system have been applied to manipulate the genome in a myriad of organisms. Recently, taking advantage of the deaminase proteins, such as Adenosine Deaminase Acting on RNA (ADAR), new tools were developed for RNA editing. In mammalian cells, there are three types of ADAR proteins, Adar1 (two isoforms, p110 and p150), Adar2 and Adar3 (catalytically inactive). The catalytic substrate of ADAR protein is double-stranded RNA, and ADAR can remove the —NH2 group from an adenosine (A) nucleobase, changing A to inosine (I). Inosine preferentially base pairs with cytosine, and therefore the cell's transcriptional and translational machinery interprets inosine as guanosine. To achieve targeted RNA editing, the ADAR protein or its catalytic domain was fused with a λN peptide, a SNAP-tag or a Cas protein (dCas13b), and a guide RNA was designed to recruit the chimeric ADAR protein to the target site. Alternatively, overexpressing ADAR1 or ADAR2 proteins together with an R/G motif-bearing guide RNA was also reported to enable targeted RNA editing.

However, currently available ADAR-mediated RNA editing technologies have certain limitations. Over-expression of ADAR1 has recently been reported to confer oncogenicity in multiple myelomas due to aberrant hyper-editing on RNAs, and to generate substantial global off-targeting edits. In addition, ectopic expression of proteins or their domains of non-human origin has potential risk of eliciting immunogenicity.

There is a need for control of the translation of nucleic acid based therapeutics. In particular, there is a need for an inducible ADAR system that avoids overexpression of ADARs and can rapidly activate ADAR to tailor the adenosine deaminase activity in a rapid and controlled manner.

### SUMMARY

Provided herein are compositions, kits, systems and methods related to an engineered, inducible adenosine deaminase (iAD) enzymes, including but not limited to, an engineered inducible adenosine deaminase acting on RNA (ADAR) enzyme, which can be activated in the presence of an inducer. Without wishing to be limited to theory, ADAR is used as an exemplary engineered inducible adenosine deaminase (iAD), but it is envisioned that the methods, compositions and systems disclosed herein are applicable to other adenosine deaminase enzymes, including but not limited to ADAR, ADAD and ADAT. Disclosed herein are inducible AR (iAR) proteins, e.g., inducible ADAR (iADAR) enzymes that can transition from an OFF (“iADAR-OFF”) to an ON (“iADAR-ON”) state in the presence of an inducer, therefore enabling rapid and controllable regulation of the adenosine deaminase activity. Also described are synthetic RNA molecules, to which the iAD can be specifically recruited to edit at least one stop codon into a non-stop codon, leading to decreased or increased translation of the RNA molecules depending on the specific construct. For example, when the iADAR is ON state, it can affect the translation of a gene of interest (GOI), depending on the target nucleic acid construct that the iADAR acts on, resulting in translation of a GOI being turned ON or OFF. By way of example only, an iADAR in the on state (iADAR-ON) can edit A→I, therefore changing a STOP (UAG) codon to UIG, therefore eliminating the STOP codon.

In one embodiment, if the STOP codon, which is present in a double stranded transcript region, herein referred to as a “ds-STOP region” is upstream (e.g., 5′) of an open reading frame (ORF), such as a GOI (referred to herein as an “target activation construct” or “TAC”), the iADAR-ON can remove the STOP codon resulting in translation of the downstream GOI. Thus, in this embodiment, in the presence of an inducer, gene translation is ON. That is—in the presence of the inducer, the translation of the GOI is switched from OFF→ON. In another embodiment, if the ds-STOP region comprising the STOP codon is located between a 5′ GOI and a 3′ polyA signal (referred to herein as an “inactivation construct” or “TIC”), an iADAR-ON can edit and remove the STOP codon, resulting in translation of the polyA tail, stalling of the ribosome, and leading to NON-STOP decay of the mRNA GOI. Thus, in this embodiment, in the presence of an inducer, gene translation is OFF. That is, in the presence of an inducer, the translation of the GOI is switched from ON→OFF. In some embodiments, the mRNA encoding the GOI is also destroyed by the cell.

In other aspects described herein are synthetic RNA molecules, to which the iAD can be specifically recruited to edit at least one start codon into a non-start codon, leading to decreased translation of the RNA molecules and/or altered translation initiation sites depending on the specific construct.

In other aspects described herein are synthetic RNA molecules, to which the iAD can be specifically recruited to edit at least one non-start codon into a start codon, leading to increased translation of the RNA molecules.

In other aspects described herein are synthetic RNA molecules, to which the iAD can be specifically recruited to edit at least one sense codon into a mutated sense codon, leading to an alteration of the structure and/or function of the RNA and/or encoded polypeptide, depending on the specific construct.

One aspect of the technology relates to an inducible adenosine deaminase enzymes (iAD), for example, but not limited to inducible ADAR (iADAR) enzymes. Other aspects disclosed herein relates to another inducible aminase enzyme, such as an inducible ADAR, ADAD or ADAT.

The technology described herein is also directed to systems comprising the iAD and synthetic RNA molecule, nucleic acids and vectors encoding the iAD and synthetic RNA molecule, and methods of using such systems, nucleic acids, and vectors.

Another aspect of the technology relates to synthetic nucleic acid constructs that iADAR effectuates.

Another aspect of the technology relates to systems and cells comprising an iADAR and a nucleic effector construct, e.g., an activation construct or inactivation construct as disclosed herein.

Another aspect relates to nucleic acid constructs that function as an activation construct or inactivation construct. Another aspect relates to nucleic acid encoding an iADAR and one or more of a target activation construct (TA-construct or TAC) or target inactivation construct (TI-construct or TIC).

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1A-1E—ADAR2-DD Can Edit Reporter UAG Stop Codons in dsRNA Hairpins. FIG. 1A) Stop codon editing reporter composed of a single mRNA transcript encoding for mCherry-FLAG (red) and HA-mNeonGreen (green) separated by a dsRNA hairpin containing a UAG stop codon (dark gray) and an RNA-binding element (light gray). In the absence of recoding, only mCherry is translated by host ribosomes. Figure discloses “AAAAAAAAAAA” as SEQ ID NO: 407. FIG. 1B) Co-expression of a fusion protein containing an RNA binding domain and a hyperactive ADAR-deaminase domain (orange and yellow) leads to colocalization of substrate & enzyme, editing of UAG stop codon to UIG, read-through by ribosome of RNA elements, and expression of mNeonGreen. Figure discloses “AAAAAAAAAAA” as SEQ ID NO: 407. FIG. 1C) Representative images of HEK293FT cells co-transfected with a UAG-MS2 editing reporter and MCP-ADAR2(E488Q)-TagBFP or catalytically inactive ADAR2(E396A). Scale-bar=250 μm. FIG. 1D) Stop codon editing assay by Western blot analysis of HEK293FT cells transfected with mRNA reporters containing different number of stop codons and/or MS2 elements. FIG. 1E) Orthogonality of ADAR2 editing of reporters with different RNA-binding domains and RNA elements observed by representative micrographs of HEK293FT cells co-transfected with reporters and ADAR variants. Images are overlays of mCherry (magenta) and mNeonGreen (green) fluorescence. Control lane were transfected only with the reporter. Scale bar=500 μm.

FIG. 2A-2D—Final Stop Codon Editing Leads to Reduced Protein Expression. FIG. 2A) Stop codon editing reporter composed of a single mRNA transcript encoding for a destabilized EGFP variant (EGFPd2—green), UAG stop codon in a dsRNA hairpin (dark gray), an RNA-binding element (light gray), and polyA tail. No other stop codons are present outside of the loop. In the absence of editing, EGFPd2 is translated by the host ribosome. Figure discloses “AAAAAAAAAAA” as SEQ ID NO: 407. FIG. 2B) Plasmid architecture of a reporter than turns off EGFPd2 with RNA editing. Bidirectional CMV (BiCMV) drives expression of a constitutive dTomato (red) and an editable EGFPd2 construct via separate transcripts. Figure discloses “AAAAAAAAAAA” as SEQ ID NO: 407. FIG. 2C) ADAR-DD leads to editing of all reporter stop codons, translation and ribosome stalling at the polyA tail, recruitment of proteins associated with non-stop decay (Ski7), and mRNA degradation by exonucleases/exosome. FIG. 2D) HEK293FT cells were transfected with inactive and active ADAR variants with the Non-Stop-Decay reporter construct and fluorescent images were collected 48 hours later. Overlay of dTomato and EGFPd2 shows relative extent of expression. Scale bar=200 μm.

FIG. 3A-3H—Engineered, Drug-Inducible ADAR2-DD by Chemical Disruption of Intramolecular Binding Domains. FIG. 3A) Crystal structure of ADAR2-DD bound to dsRNA (PDB-5ED2). RNA shown in orange, ADAR2-DD shown in cyan, 5′ Binding Loop residues amenable to insertions shown in green and the C-terminus shown in red. FIG. 3B) A model of an autoinhibitory ADAR: an insertion is made in the 5′ Binding loop (green) that does not disturb catalytic activity. Subsequent fusion of a high affinity binding partner (gray) to the C-terminus (red) leads to an equilibrium shift towards an inhibited state. The addition of a small-molecule drug that can disrupt that interaction (orange) leads to an active ADAR-DD state. FIG. 3C) Architecture of the drug-inducible ADAR proteins using BH3 derived peptides and Bcl-2 Homologs as the interaction domains. FIG. 3D) Representative fluorescent micrographs showing the relative expression of mCherry and mNeonGreen from the ADAR-editing reporter when HEK293FT cells were co-transfected with different ADAR variants. Merged channels show overlay of mCherry (magenta) and mNeonGreen (green). Table below micrographs identify the ADAR variant by BH3-peptide insertion at 5′ Binding Loop (Bad, Bim, or MS1(I17A)) and Bcl-2 Homolog fused at the C-terminus (Bcl-xL or Mcl-1), and displays whether inhibitory drugs were added. Scale bar=200 μm. FIG. 3E) Flow cytometry analysis of HEK293FT cells transfected with reporter and ADAR variants. ADAR2-BclxL represents Bad insertion variant. Drug added for BclxL was 500 nM of A-1331852 at the time of transfection, and for Mcl-1 was 2 μM of S63845. Cells were gated for ADAR transfection via BFP fluorescence, and relative fluorescence was quantified by the median of the ratio of mNeonGreen to mCherry. Bars represent mean fluorescence of three independent transfections (n=3). FIG. 3F) Drug response titration of Bad-BclxL system using A-1331852. Bad point mutant was also tested (F121L). Drug added at the time of transfection (other than Cntrl condition, where no drug was added). Relative fluorescence quantified via flow cytometry 48 hours after transfection. Mean relative fluorescence for three independent transfections (n=3) is plotted ±s.d. per drug concentration. FIG. 3G) Drug response titration of MS1-Mcl-1 system using S63845. Original MS1 (I17) and destabilized binding mutant MS1 (I17A) were both tested. Drug added at the time of transfection (other than Cntrl condition, where no drug was added). Relative fluorescence quantified for transfected cells via flow cytometry 48 hours later. Mean relative fluorescence for three independent transfections (n=3) is plotted ±s.d. per drug concentration. FIG. 3H) Flow cytometry analysis of HEK293FT cells transfected with variants of ADAR and with the EGFPd2/dTomato reporter, where editing leads to destruction of fluorescent signal. If indicated, 2 μM of S63845 was added at the time of transfection. Mean relative fluorescence for three independent transfections (n=3) is plotted and P-values that were derived from a 2-way ANOVA (groups were ADAR variant and drug) are shown for certain comparisons.

FIG. 4A-4E—Autoinhibited ADAR Variants Utilizing Repressive Epitope-Antibody Fragment Interactions Can Activate via Antigen Binding. FIG. 4A) A model of allosteric ADAR activation via competitive antigen binding. An inserted epitope at the 5′ binding loop (green) and a C-terminally (red) fused antibody fragment (gray) bind and make the ADAR adopt an inhibited state. Soluble antigen (purple) that can compete with the intramolecular interaction due to high concentration or affinity shifts the equilibrium towards an active ADAR, allosterically activating the ADAR. FIG. 4B) Crystal structure of the ALFA epitope tag and anti-ALFA nanobody (NbALFA) (PDB—6I2G). FIG. 4C) Protein architecture of ALFA-based allosteric ADAR and soluble ALFA antigen fused to miRFP. Also displayed are the amino acid sequences of ALFA variants with lowering affinity (ALFA: SRLEEELRRRLTE, SEQ ID NO: 85; AFLA-PE: GRLEEELRRRLSP, SEQ ID NO: 86; ALFA-78: GRLEQEIRARLSP, SEQ ID NO: 87). FIG. 4D) Two-dimensional contour plots of mNeonGreen vs mCherry fluorescence derived from flow-cytometry analysis of transfected HEK293FT cells (gated by BFP and mCherry). Each contour-group contains 10% of the population. Original full length ADAR-DD and catalytically inactive E396A mutant shown on left (blue and red), and ALFA insertion with NbALFA fusion without and with soluble ALFA shown on right (green and purple). Each population displays an individual replicate representative of an experiment done in triplicate. FIG. 4E) Representative fluorescent micrographs showing the relative expression of mCherry and mNeonGreen from the ADAR-editing reporter when HEK293FT cells were co-transfected with different ALFA-ADAR variants with and without soluble ALFA co-transfection. Merged channels show overlay of mCherry (magenta) and mNeonGreen (green). The table below the micrographs identify the ADAR variant by ALFA variant insertion at 5′ Binding Loop (ALFA, ALFA-PE, or ALFA-78) and whether NbALFA was fused to the C-terminus, and displays whether miRFP670-ALFA was co-transfected. Scale bar=500 μm.

FIG. 5A-5G—Autoinhibited ADAR Can Be Activated Through Proteolytic and Photolytic Cleavage. FIG. 5A) A model of proteolytic cleavage based induction of ADAR activity. In this scheme, a cut site (yellow) is inserted in the linker between the C-terminus of ADAR (red) and the N-terminus of a protein domain (gray) that constitutively binds a peptide/protein inserted at the 5′ Binding Loop (green). Irreversible proteolytic cleavage does not interfere with protein interactions but leads to relaxation of the autoinhibited state and therefore catalytically active ADAR. FIG. 5B) Crystal structure of the SpyTag (green) and SpyCatcher (gray) covalent complex (PDB—4MLI). FIG. 5C) Architecture of the ADAR that can be activated by TEV protease used in subsequent experiments. FIG. 5D) Flow cytometry analysis of HEK293FT cells transfected with editing reporter and SpyTag based ADAR variants. Conditions related to ADAR variant and TEV addition shown below (2A refers to SpyCatcher being co-expressed via 2A self-cleaving peptides and not C-terminally fused like the others). Cells were gated for ADAR transfection via BFP fluorescence, and relative fluorescence was quantified by the median of the ratio of mNeonGreen to mCherry. Bars represent mean fluorescence of three independent transfections (n=3) ±s.d., and P-Values displayed were derived from one-way ANOVA. FIG. 5E) Two-dimensional contour plots of mNeonGreen vs mCherry fluorescence derived from flow-cytometry analysis of ADAR-TEVcs transfected HEK293FT cells gated by BFP and mCherry (right two columns from D). Each contour-group contains 10% of the population. Co-transfected with a plasmid encoding TEV protease shown in red, whereas transfected without TEV protease is shown in blue. Each population displays an individual replicate representative of an experiment done in triplicate. FIG. 5F) A model for photolytic activation of ADAR variants. Similar to model A, but a photocleavable domain (i.e., PhoCl) is inserted between ADAR and the binding domain instead of a protease cleavage site. Irreversible photocleavage via purple light does not interfere with protein interactions but leads to relaxation of the autoinhibited state and therefore catalytically active ADAR. FIG. 5G) Fluorescence micrographs showing an increase in relative fluorescence of mNeonGreen (green) and mCherry (magenta) over time in cells that were transfected with the editing reporter and a PhoCl integrated Bad-BclxL construct. At time 0, cells were imaged and then illuminated with violet light from a BFP filter for 10 s. 2 hours later, the same spot was recorded and illuminated again with 10 s of violet light before being recorded for a final time 4 hours from the first illumination event. Scale bar=500 ρ.

FIG. 6A-6O are tables showing the domains of the polypeptides of SEQ ID NOs:1-37. “SID” indicate the SEQ ID NO. See also Table 1 in Example 3.

FIG. 7A-7B shows a sequence alignment of ADAR1 (DSRAD; SEQ ID NO: 79), ADAR2 (RED1; SEQ ID NO: 80), and ADAR3 (RED2; SEQ ID NO: 81).

FIG. 8A-8C shows a sequence alignment of ADAR1 (DSRAD; SEQ ID NO: 79), ADAR2 (RED1; SEQ ID NO: 80), ADAR3 (RED2; SEQ ID NO: 81), ADAD1 (SEQ ID NO: 82), and ADAD2 (SEQ ID NO: 83).

FIG. 9A-9C shows a sequence alignment of ADAT1 (SEQ ID NO: 84), ADAR1 (DSRAD; SEQ ID NO: 79), ADAR2 (RED1; SEQ ID NO: 80), ADAR3 (RED2; SEQ ID NO: 81), ADAD1 (SEQ ID NO: 82), and ADAD2 (SEQ ID NO: 83).

FIG. 10 shows a phylogenetic tree of ADAT1 (SEQ ID NO: 84), ADAR1 (DSRAD; SEQ ID NO: 79), ADAR2 (RED1; SEQ ID NO: 80), ADAR3 (RED2; SEQ ID NO: 81), ADAD1 (SEQ ID NO: 82), and ADAD2 (SEQ ID NO: 83).

FIG. 11A-11F—Fusion of heterodimers to the N and C termini leads to allosteric ADARs. FIG. 11A) General map of previous topology for creating autoinhibited ADAR enzymes. Here two heterodimeric protein components (A and B) are inserted at a specific loop and fused to the C-terminus. FIG. 11B) Quantification of fluorescent micrographs demonstrating that the second protein partner must be fused to the C-terminus. HEK cells were transfected with 50 ng of both a reporter construct and of an ADAR construct and treated with A-1331852, and two days later images were taken on an epifluorescent microscope. The images were then analyzed by the following—background was subtracted, a mask was created of transfected cells using the BFP channel, and the ratio of mNeonGreen to mCherry of the corresponding region was computed with ImageJ. FIG. 11C) ADAR2-DD crystal structure (PDB 5ED2). The C-terminus is shown in red and the insertion loop is shown in green. The distance between the two is greater than 50 Λ. FIG. 11D) Map of new autoinhibited ADAR constructs with the heterodimeric protein components fused to the N and C termini. FIG. 11E) Crystal structure showing the distance between the C (red) and N (green) termini is greater than 50 Å when folded. FIG. 11F) Quantification of fluorescent micrographs demonstrating that using both termini can lead to an allosterically activated ADAR construct. HEK cells were transfected with 50 ng of both a reporter construct and of an ADAR construct and treated with A-1331852, and two days later images were taken on an epifluorescent microscope. The images were then analyzed by the following—background was subtracted, a mask was created of transfected cells using the BFP channel, and the ratio of mNeonGreen to mCherry of the corresponding region was computed with ImageJ. Please note that this experiment was done with the experiment for FIG. 11B, and that some data points are the same. All data shown is n=1.

FIG. 12A-12D—Single plasmid constructs which encode a self-editing mRNA leads to efficient activation. FIG. 12A) General schematic for how to create a self-editing, ADAR-encoding mRNA. Upstream of an editable stop codon, an allosteric ADAR is fused to an RNA-binding protein that recognized a motif adjacent to the first stop codon. Downstream of the stop codon is a gene of interest. FIG. 12B) Schematics for testing self-editing mRNA used in subsequent experiments. BAD(F)-Bcl-xL were used as the pair, and either 1 or 2 editable stop codon loops (each containing two UAG stop codons) were downstream. FIG. 12C) Fluorescent micrographs of HEK cells which were transfected two days prior with each construct listed above (corresponding to whether the ADAR was mutated and whether there were one or two editable stop codon loops) with or without 1 uM of A-1331852. mCherry is shown in red and mNeonGreen is shown in green as a single image. Robust editing as seen by mNeonGreen fluorescent is apparent in both configurations. FIG. 12D) Quantification of the images shown in FIG. 12C. The images were then analyzed by the following—background was subtracted, a mask was created of transfected cells using the mCherry channel, and the value of mNeonGreen of the corresponding region was computed with ImageJ. All data shown is n=1.

FIG. 13A-13B show schematics of embodiments that can be delivered as a single mRNA therapeutic. For example, the RNA molecule and fusion protein components can be combined into a single deliverable. FIG. 13A shows that a Ribonucleoprotein complex can comprise a pre-assembled engineered ADAR sensor with mRNA. FIG. 13B shows that a single, self-editing mRNA construct can encode the ADAR component upstream in the first open reading frame and a downstream product (e.g., an effector protein such as a reporter, interferon, caspase, etc.) in the second open reading frame of the RNA.

FIG. 14A-14C are tables showing the domains of the polypeptides of SEQ ID NOs: 88-92. “SID” indicate the SEQ ID NO. See also Table 2 in Example 7.

FIG. 15A-15B are tables showing the domains of the polypeptides of SEQ ID NOs: 93-94. “SID” indicate the SEQ ID NO. See also Table 3 in Example 8.

FIG. 16A-16B are schematic illustrations to show the modification of the deaminase domain of adenosine deaminases, including ADAR, into an inducible system and function to change a stop codon on exemplary synthetic activation or inactivation constructs. FIG. 16A is a schematic of one embodiment, showing modification of the Deaminase domain (DD) so that the adenosine deaminase activity is constitutively on. In the embodiment shown, the DD is a heterodimer of two fragments or portions, e.g., AD-DDn and AD-DDc, however, it is envisioned that the DD can be a single polypeptide that is not split. The Table in FIG. 16A shows that when the constitutively active modified AD, e.g., ADAR is coupled with an affinity binding pair as disclosed herein, it becomes an inducible AD (iAD) or inducible ADAR (iADAR), and depending on the location of the ds-STOP region, will result in activation of a GOI or deactivation (e.g., mRNA decay) of a GOI. FIG. 16B is a schematic illustration of the iADAR fusion protein that comprises an affinity binding pair (e.g., BP1 and BP2), that when in the absence of an inducer prevents the co-factor IP6 from activating the adenosine deaminase activity. In the presence of an inducer, the binding between the affinity binding pair (e.g., BP1 and BP2) is interrupted or inhibited, thereby allowing IP6 to bind to the DD and changing the iADAR from the OFF to ON state, and adenosine deaminase activity can occur. Depending on the location of the ds-STOP region in a target construct, e.g., a target activation construct (TAC) or a target inactivation construct (TIC), the GOI expression is turned ON or OFF respectively. Depending on the affinity binding pair of the iADAR, inducers can be, but are not limited to, small molecules, proteases, light-inducible control, sound inducible control, cell cycle dependent, ultrasound or other wavelength dependent, antibodies, endogenous triggers, disease triggers, external triggers and cell-specific marker triggers, and the like.

FIG. 17 shows activity of a Grazoprevir Activated ADAR (“AD-Pep-AD-NS3”).

FIG. 18 shows SEQ ID NO: 168 (AD-Pep-AD) and SEQ ID NO: 169, exemplary iADARs using NS3 and NS3 peptide.

FIG. 19A-19B shows that fusion of an additional binding domain localizes inducer to iADAR and increases sensitivity.

FIG. 20A-20H show non-limiting examples of dsRNA stop loops with RNA motifs; see also Example 1. The RNA secondary structures were generated by RNAFold™. In FIG. 20A-20G, the yellow loop is the dsRNA stop loop, and the blue loop is the dsRNA binding motif (e.g., MS2, PP7, HIV tar, BoxB loops), which are capable of being bound by an RNA-binding domain. FIG. 20A shows UAG-UAG Stop Loop w/MS2 Loop (SEQ ID NO: 395). FIG. 20B shows UAG-UGG Stop Loop w/MS2 Loop (SEQ ID NO: 396). FIG. 20C shows UGG-UAG Stop Loop w/MS2 Loop (SEQ ID NO: 397). FIG. 20D shows UAG-UAG Stop Loop w/Internal MS2 Loop (SEQ ID NO: 398). FIG. 20E shows UAG-UAG Stop Loop w/PP7 Loop (SEQ ID NO: 399). FIG. 20F shows UAG-UAG Stop Loop w/HIV Tar Loop (SEQ ID NO: 400). FIG. 20G shows UAG-UAG Stop Loop w/BoxB Loop (SEQ ID NO: 401). FIG. 20H shows the General Secondary Structure of dsRNA Stop Loop; the dashed lines represent hydrogen bonding between base pairs, and w, x, y & z represent variables. It should be noted that not necessarily every hydrogen bond/base pairing depicted in the diagram below needs to be maintained, but enough to become a substrate for ADAR deaminase domains. Figure discloses SEQ ID NO: 408.

FIG. 21A-21E show exemplary sequences described herein (see e.g., Example 17). FIG. 21A shows CP-linker-BclxL—linker-ADAR2-DDN-Bad(L)-ADAR2(E488Q)-DDC-TagBFP (see e.g., SEQ ID NO: 198). FIG. 21B shows MCP-linker-BAD-ADAR2-DD(E488Q)-TagBFP (see e.g., SEQ ID NO: 200). FIG. 21C shows MCP-linker-BAD-ADAR2-DD(E488Q)-Bcl-xL-TagBFP (see e.g., SEQ ID NO: 202). FIG. 21D shows tdMCP_ADAR2-DDN-CP5-46-4D5E_ADAR2-DDC(E488Q)_mTagBFP (AD-Pep-AD) (see e.g., SEQ ID NO: 204) FIG. 21E shows tdMCP_ADAR2-DDN-CP5-46-4D5E ADAR2-DDC(E488Q)_NS4A/NS3(Genotype 1B)_mTagBFP (see e.g., SEQ ID NO: 206).

FIG. 22A-22D show that mutation of IP6 binding pocket reduces the background of allosteric ADAR. FIG. 22A) A thermodynamic model showing the competition between IP6 binding and cis-heterodimerization. ADAR is shown in blue, with the C-terminus shown in brighter blue. Each dimer component is shown in green and red. IP6 is shown in magenta. The affinity of each component shifts the equilibrium accordingly. FIG. 22B) Residues that contact IP6 and/or stabilize the C-terminus are shown in the crystal structure (left) or as a LigPlot (right). FIG. 22C) A plasmid map of the construct that was used in the mutational screen. BAD(V), a mutant with lowered affinity to Bcl-xL, was used because of its leakiness. FIG. 22D) Flow cytometry data of HEK cells that were co-transfected with an ADAR reporter and ADAR mutant variants of BAD(V)-BclxL in the presence of A-1331852. Two days after transfection, cells were lifted and analyzed via flow cytometry. Using FLOWJO, cells were gated by 1% of BFP fluorescence and the median of the ratio of mNeonGreen to mCherry is plotted. All data shown is n=1.

FIG. 23A-23E show that IP6 binding mutations decrease the background of MS1 and of N-terminal BAD ADAR variants. FIG. 23A) Plasmid map of MS1(I17A)/MS1(I17G)-Mcl1 ADAR variants. FIG. 23B) Quantification of fluorescent micrographs of MS1(I17A)-Mcl1 ADAR mutants. HEK cells were transfected with 50 ng of both a reporter construct and different ADAR mutant constructs and treated with S63845 (an Mcl-1 inhibitor), and two days later images were taken on an epifluorescent microscope. The images were then analyzed by the following—background was subtracted, a mask was created of transfected cells using the BFP channel, and the ratio of mNeonGreen to mCherry of the corresponding region was computed with ImageJ. FIG. 23C) Quantification of fluorescent micrographs of MS1(I17G)-Mcl1 ADAR mutants. HEK cells were transfected with 50 ng of both a reporter construct and different ADAR mutant constructs and treated with S63845 (an Mcl-1 inhibitor), and two days later images were taken on an epifluorescent microscope. The images were then analyzed by the following—background was subtracted, a mask was created of transfected cells using the BFP channel, and the ratio of mNeonGreen to mCherry of the corresponding region was computed with ImageJ. FIG. 23D) Plasmid map of N-terminal BAD fusion construct. FIG. 23E) Quantification of fluorescent micrographs of nBAD-ADAR-cBcl-xL mutants. HEK cells were transfected with 50 ng of both a reporter construct and different ADAR mutant constructs and treated with A-1331852 (a Bcl-xL inhibitor), and two days later images were taken on an epifluorescent microscope. The images were then analyzed by the following—background was subtracted, a mask was created of transfected cells using the BFP channel, and the ratio of mNeonGreen to mCherry of the corresponding region was computed with ImageJ. All data shown is n=1.

FIG. 24 shows amino acid residues in the IP6 binding pocket of ADAR.

FIG. 25A-25C show exemplary sequences described herein (see e.g., Example 17). FIG. 25A shows MCP-linker-ADAR2-DDN-Bad(F)-ADAR2(E488Q)-DDC-Bcl-xL (see e.g., SEQ ID NO: 287) and MCP-linker-ADAR2-DDN-Bad(F)-ADAR2(E488Q)-DDC-Bcl-xL-TagBFP (see e.g., SEQ ID NO: 288). FIG. 25B shows MCP-linker-ADAR2-DDN-Bad(F)-ADAR2(E488Q)-DDC-Bcl-xL-TagBFP (see e.g., SEQ ID NO: 289) and MCP-linker-ADAR2-DDN-MS1(A)-ADAR2(E488Q)-DDC-TagBFP (see e.g., SEQ ID NO: 290). FIG. 25C shows MCP-linker-ADAR2-DDN-MS1(G)-ADAR2(E488Q)-DDC-TagBFP (see e.g., SEQ ID NO: 291).

FIG. 26A-26E show editing of an upstream “AUA” to “AUI” for defining a new start codon and open reading frame (ORF). Creation of a new start codon and ORF by editing mediated conversion of a 5′ non-ORF target site. FIG. 26A) General schematic for ADAR-editing creation of a novel start codon. An RNA target substrate in the 5′UTR of a transcript contains an editable AUA target positioned in frame with a downstream ORF (EGFPd2. Co-expression of RBP-ADAR-DD leads to editing of the AUA into AUI, generating a start codon that can be interpreted as “AUG.” FIG. 26B; SEQ ID NO: 409) Sequence and secondary structure prediction of an example editable substrate as predicted by RNAfold. An MS2 motif for interaction with an MCP based RBP is highlighted. The AUA editing target is also highlighted. Conversion of this AUA into AUI generates a new start site and ORF. FIG. 26C) General plasmid map of the tested design. FIGS. 26D&26E) HEK cells were co-transfected with a plasmid encoding a bidirectional CMV promoter encoding an editing target in combination with dTomato as a transfection marker. Two editing target constructs are compared, which contain either a high-affinity “MS2-C” (FIG. 26D) or modest affinity “MS2-A” (FIG. 26E) MCP/RBP sequence⁴. Editable sequences containing “AUA” targets within the RNA motifs were tested in combination with active or inactive iADAR constructs. Levels of editing were determined by measuring EGFPd2/dTomato ratios. The AUA-containing “editable” targets were compared to control sequences containing the intended editing product (“AUG”) as a positive control. Control cells expressing the reporter without transfected iADAR constructs (“NT”—white); cells expressing the reporter in combination with an inactive MCP-ADAR(E396A) (“dADAR”—striped); cells expressing reporter in combination with active MCP-ADAR (“ADAR”—black) deaminase domains. Cells were analyzed by flow cytometry with quantification of dTomato and EGFP2 levels in single cells; plotted values represent median emission value intensities of the analyzed populations (EGFPd2:dTomato). Positively transfected cells were gated based on dTomato expression. Statistical significance determined using Prism: Two-way ANOVA (n=3 separate transfections). ****—P<0.0001, ***—P<0.001, **—P<0.01, *—P<0.05.

FIG. 27A-27D show upstream AUA to AUI editing to create an expanded ORF encoding a protein with an editing-dependent N-terminal fusion. ORF expansion was accomplished by editing of in-frame and upstream editing of AUA to an AUI. FIG. 27A) General schematic of iADAR-mediated editing of a non-coding AUA target for creation of a new (AUI) start codon and ORF. Conversion of the target AUA into AUI results in the in-frame fusion of target protein with a signal sequence (SS) for ER-mediated protein secretion in combination with an HA epitope tag. A RNA target in the 5′UTR contains an editable AUA sequence which is positioned in frame with the original start codon encoding a cytoplasmically localized GFP protein. Co-expression of an RBD-ADAR-DD leads to the creation of an upstream start codon (AUI) that leads to an ORF expansion and the encoding of a protein product containing an N-terminally fused signal peptide. Note that upon creation of the AUI new start codon, the original AUG start codon becomes read as an elongator AUG/methionine. Note also that the editing-mediated ORF expansion results in the secretion of the encoded protein into the ER lumen via the translated signal sequence. Secretion of this protein also permits the post-translational modification of GFP which contains a C-terminal site for GPI (glycosylphosphatidylinositol) linkage. GPI modification is not expected to occur for the non-edited cytoplasmic GFP, as modification with this lipid does not generally occur cytoplasmically. Thus the net result of the editing event produces a new protein product with i) higher mass, ii) altered localization, iii) altered recognition (HA tag), and iv) altered post-translational modification susceptibility. FIG. 27B) Schematic for the cell-based detection of editing-mediate protein relocation. Non-transfected cells (left) do not express any GFP, transfected cells without ADAR-editing of new start codon express GFP intracellularly (middle). Upon ADAR editing of the targeted AUA, a new start codon is generated and the ORF is expanded. The protein product of the expanded ORF encodes a secretory pathway-targeted, HA-tagged, and GPI modified protein. Thus, following editing, GFP could be localized to luminal and extracellular positions in combination with intracellular localized GFP translated from unedited or pre-edited transcripts (right). FIG. 27C) Imaging of live HEK293FT cells transfected with plasmids encoding the edit-target containing GFP-GPI encoding transcript. Cells co-transfected with plasmids encoding editing inactive (top) or active MCP-ADAR (bottom) constructs are shown. AUA-SS-GFP-GPI co-transfected with inactive dADAR showed primarily cytoplasmic localization of GFP, whereas active ADAR showed membrane and ER localization (white arrows). Scale bar is 100 μm. FIG. 27D) Confirmation of the editing-induced altered GFP localization and HA-fusion by antibody staining of the HA epitope occurs only in active MCP-ADAR transfected cells. Transfected cells were fixed and stained for HA epitope tag using an anti-HA antibody and fluorescent AF647 conjugated secondary antibody. The constitutively exported SS-HA-GFP-GPI control and MCP-ADAR conditions had anti-HA AF647 signal that colocalized with GFP signal at the plasma membrane. In contrast, cells transfected with inactive MCP-dADAR contained minimal anti-HA signal. Scale bar—50 μm.

FIG. 28A-28D show AUG to IUG editing for start codon removal and ORF modification/elimination. Functional start codons were converted to non-functional start codons with cis-acting ADAR deaminase domains. FIG. 28A) General schematic for iADAR mediated elimination of a start codon. An editable RNA motif containing a targeted start codon (AUG) is positioned within the 5′ region of a EGFPd2-encoding ORF. Upon editing, the AUG target is converted to JUG, eliminating its recognition by translation initiation machinery and thereby altering/eliminating the EGFPd2-encoding ORF. In cells without iADAR, or prior to/in the absence of editing EGFPd2 will be translated in full; following editing by RBP-ADAR-DD translation of full length EGFPd2 is blocked. FIG. 28B) Secondary structure prediction of an editable loop by RNAfold. MS2 motif is shown in yellow and the AUG start codon is shown in green. Figure discloses SEQ ID NO: 410. FIG. 28C) General plasmid map depicting the reporter scheme. FIG. 28D) Transfection of HEK cells with editable AUG reporters containing MS2-C or MS2-A motifs and active ADAR leads to a significant decrease in translational efficiency of downstream EGFPd2 compared to inactive MCP-dADAR. Cells were transfected with the two reporters and either non-ADAR encoding DNA (NT—white), inactive MCP-dADAR (dADAR—striped), or active MCP-ADAR (ADAR—black). 48 hours post-transfection, cells were trypsinized and analyzed by flow cytometry. Values are plotted as median levels of relative EGFPd2-to-dTomato emissions. Transfected cells were identified based on dTomato gating. Statistical analysis for significance performed via Prism: Two-way ANOVA (n=3 separate transfections). ****—P<0.0001, ***—P<0.001, **—P<0.01, *—P<0.05.

FIG. 29A-29C show Sense Codon Editing for altering the localization, fusion state, and activity of an mRNA encoded protein (AGG to IGG). Sense codon editing of RNA regions encoding a 2A “skipping” peptide results in altered protein targeting, localization, and activity. FIG. 29A) Schematic of sense codon ADAR editing DNA construct. A dsRNA hairpin is inserted at the C-terminus of a skipping deficient T2A-G18R mutant with a MS2-C loop. In the presence of ADAR activity, the Gly18 is rescued by deamination of the AGG codon to IGG. FIG. 29B) Multiple-sequence alignment of various known 2A “self-cleaving” or “skipping” peptides. Sequences from different viruses are shown, including: P2A, porcine teschovirus-1 (SEQ ID NO: 360); T2A, thosea asigna virus (SEQ ID NO: 361); E2A, equine rhinitis A virus (SEQ ID NO: 362); F2A, foot-and-mouth disease virus (SEQ ID NO: 363). Conserved residues that are needed for the “skipping” activity of these 2A peptides are highlighted. Mutation of these residues eliminates self-cleavage/skipping activity, resulting in the translation of an unskipped (intact) fusion protein. FIG. 29C) Reporter design for editing-induced formation of a skipping peptide. In this design, iADAR activity is utilized to convert a mutated 2A sequence into a skipping active 2A peptide. In the absence of ADAR editing/prior to editing (left), an N-terminal secretion/signal-sequence targets the intact full-length protein into the ER, including the Gal4-VP64 transcription factor sequence. Upon ending of a target codon an active 2A peptide is generated, resulting in the skipping and release of a cytoplasmic Gal4-VP64, which can then be translocated to the nucleus to activate a target gene. Generation of the active 2A peptide is mediated by base editing of an arginine encoding send codon (AGG) to a sense codon that is interpreted as a glycine (IGG). IGG (right), which is read as a glycine, the Gal4-VP64 is now expressed in the cytoplasm where it can translocate into the nucleus and turn on an H2B-mCherry reporter that is integrated with upstream UAS elements.

FIG. 30A-30F show Two-Input, dual-editing, AND-gate mRNA editing substrates. Multiple stop codons and RNA-binding motifs enable multi-input logic. FIG. 30A) Schematic of 4×UAG MS2-C ADAR-dependent reporter construct. FIG. 30B) Schematic of novel dual-input mRNA reporters. 2 upstream, editable stop codons have an MS2 motif, and the subsequent 2 stop codons have a different RNA motif (PP7, BoxB or HIV-TAR). FIG. 30C) 4×UAG-MS2-C reporter expression of mNeonGreen is dependent on active ADAR. HEK cells co-transfected with the reporter and either non-ADAR coding DNA or MCP-ADAR. FIG. 30D-30F) 2×UAG-MS2 and 2×UAG-PP7 reporter (FIG. 30D) has full expression when co-transfected with MCP-ADAR and PCP-ADAR. However, BoxB (FIG. 30E) and HIV-TAR (FIG. 30F) constructs did not show significant improvement in dual transfection compared to MCP-ADAR transfection. HEK cells co-transfected with the different reporters and different RBD-ADAR constructs. 48 hours post-transfection, cells were lifted, flow cytometry was performed and median relative mNeonGreen-to-mCherry fluorescence was computed for transfected cells based on mCherry gating. Statistical analysis for significance performed via Prism: One-way ANOVA (n=3 separate transfections). ****—P<0.0001, ***—P<0.001, **—P<0.01, *—P<0.05.

FIG. 31A-31C show editing of an internal STOP codon between fusion-dependent protein domains. Directed ADAR editing of internal STOP codons in a protein can rescue function. FIG. 31A) General schematic of an internal STOP iADAR product. Here, two polypeptide sequences (red and green) whose sequences must be fused to be functional are separated by a STOP codon and RNA binding motif (RBM). This scheme can be used for split-proteins like a split fluorescent protein (left) or multi-domain proteins like transcription factors (middle) or membrane receptors (right). FIG. 31B) Plasmid map of internal STOP codon reporter construct tested. There are two upstream, editable STOP codons, and one STOP-MS2 loop inserted in the mNeonGreen protein. Editing of all 3 STOP codons would be necessary for rescue of mNeonGreen fluorescence. FIG. 31C) The Internal STOP reporter construct functions as expected, where mNeonGreen expression is stimulated by co-transfection with active MCP-ADAR. HEK293FT cells were co-transfected with the original 4×UAG-MS2 reporter or the Internal STOP reporter and either non-coding DNA, inactive MCP-dADAR (dADAR), or active MCP-ADAR (ADAR). Relative fluorescence is diminished for the Internal-STOP compared to the 4×UAG-MS2 reporter, but is significantly increased when co-expressed with active ADAR. 48 hours post-transfection, cells were lifted, flow cytometry was performed and median relative mNeonGreen-to-mCherry fluorescence was computed for transfected cells based on mCherry gating.

FIG. 32A-32D show ADAR2-DD Mutations with ALFA-Sensing iADAR. ADAR2 mutations increase fold change of weaker antigen sensing systems. FIG. 32A) Map of previously tested ALFAtag iADAR, where the intramolecular interaction between ALFA epitope variants and the AlfaNb autoinhibit the deaminase activity. FIG. 32B) Map of newly tested constructs, which contain mutations to the ADAR2-DD and also include a GFP nanobody to improve co-localization of the activating EGFP(R96M)-ALFAtag. FIG. 32C& FIG. 32D) HEK293FT cells were transfected with 4×UAG MS2 Reporter, either EGFP(R96M) or EGFP(R96M)-ALFAtag, and ADAR2-DD mutants for the high affinity ALFA insertion (FIG. 32C) or lower affinity ALFA-PE peptide variant (FIG. 32D). Increasing the strength of the mutation in ALFA-PE constructs leads to an increased fold change. Fluorescence was measured via microscopy and relative fluorescence per cell was computed in ImageJ. Each point represents a single cell (n=1 transfection).

FIG. 33A-33C show iADAR Based Antigen AND Drug Logic. Antigen, Drug AND-Gates can be constructed using dual repressed iADAR proteins. FIG. 33A) Schematic of dual input iADAR proteins. One deaminase domain contains two intramolecular interactions (gray and green, dark gray and dark green) which can lead to autoinhibition of the ADAR independently. Adding antigen (purple) or drug (orange) alone relieves one set of autoinhibitory domains, but addition of both is necessary to activate the protein. FIG. 33B) Plasmid map of ALFA-Bcl dual input iADAR. BAD peptide (dark green) is fused to the N-terminus of the ADAR2-DD(F697Y), whereas ALFA-PE is inserted at the 5′ RNA binding site (green). There is also a tandem fusion of the AlfaNb and Bcl-xL at the C-terminus. FIG. 33C) The dual input of antigen and drug leads to highest translational efficiency. HEK293FT cells were transfected with the ALFA-Bcl iADAR and the 4×UAG MS2 reporter with either EGFP(R96M) or EGFP(R96M)-ALFA and treated with either 1 μM of A-1331852 or DMSO. Significantly, the highest expression of mNeonGreen is seen with dual addition of drug and antigen. 48 hours post-transfection, cells were lifted, flow cytometry was performed and median relative mNeonGreen-to-mCherry fluorescence was computed for transfected cells based on mCherry gating. Statistical analysis for significance performed via Prism: One-way ANOVA (n=3 separate transfections). ****—P<0.0001, ***—P<0.001, **—P<0.01, *—P<0.05.

FIG. 34A-34C show Grazoprevir-Inducible iADAR by High Affinity Peptide Based Autoinhibition. The high affinity interaction between HCV NS3(1B) protease and a binding peptide leads to antiviral drug induced iADAR. FIG. 34A) Drug inducible iADAR scheme based on intramolecular interactions. FIG. 34B) Construct maps for tested iADAR variants. Pep is inserted at the 5′ RNA binding site (green) and the NS3(1B) protease domain (red) is fused to the C-terminus of the deaminase domain. FIG. 34C) Grazoprevir can induce iADAR constructs. HEK293FT cells were transfected with the NS3 iADAR variants and the 4×UAG MS2 reporter with either 2 μM of grazoprevir or DMSO added at the time of transfection. Increased repression is seen in the K690R mutant, leading to slightly elevated fold-change. 48 hours post-transfection, cells were lifted, flow cytometry was performed and median relative mNeonGreen-to-mCherry fluorescence was computed for transfected cells based on mCherry gating. Statistical analysis for significance performed via Prism: Two-way ANOVA (n=3 separate transfections). ****—P<0.0001, ***—P<0.001, **—P<0.01, *—P<0.05.

FIG. 35A-35C show Grazoprevir-Inducible ADAR by Active Proteolysis. Ligand Inducible Connection (LiNC) of cleavage labile ADAR domain creates a functional iADAR. FIG. 35A) Schematic of ADAR-LiNC. The NS5A/5B protease cut site (green) is inserted in the ADAR2-DD (blue) at the 5′ RNA binding site, and NS3 protease domain (red) is fused to the C-terminus. In the absence of drug (top), cis-proteolysis leads to inactivation of ADAR by dissociation of the two halves of ADAR2-DD. Protease inhibitor addition (bottom) ablates cleavage, leading to correct folding of ADAR-DD and deaminase activity. FIG. 35B) Construct maps of ADAR-LiNC system. dNS3 represents a catalytically inactive protease domain as a control, which is achieved through a S139A mutation. FIG. 35C) ADAR-LiNC leads to another mechanism of grazoprevir-inducible ADAR activity. A higher fold change between the uninduced and induced condition is observed for ADAR2-DD mutants (L699G and F697Y). HEK293FT cells were transfected with the LiNC iADAR variants and the 4×UAG MS2 reporter with either 2 μM of grazoprevir or DMSO added at the time of transfection. 48 hours post-transfection, cells were lifted, flow cytometry was performed and median relative mNeonGreen-to-mCherry fluorescence was computed for transfected cells based on mCherry gating.

FIG. 36A-36E show that IRES-based iADAR Constructs Enable Novel Single Construct Design. Use of the EMCV IRES leads to robust, single transcript circuits. FIG. 36A) Map of previous iterations of single-construct designs where the iADAR sensor (blue) is translated upstream of the editable STOP codons before a regulatable payload (green). FIG. 36B) Map of novel single-construct design where the iADAR sensor is driven by a downstream IRES element. Canonical translation leads to the production of a constitutive component (red) and a regulatable downstream component. FIG. 36C) HEK293FT cells were transfected with the IRES iADAR constructs expressing Bcl-xL-BAD variants with 1 μM of A-1331852 or DMSO added at the time of transfection. FIG. 36D) Map of Drug/Protease OR-gate IRES iADAR utilizing Bcl-xL, BAD, and TEVcs. Addition of drug or proteolysis will lead to release of autoinhibition. FIG. 36E) HEK293FT cells were transfected with the Bcl-TEV IRES iADAR and either filler DNA or a plasmid encoding TEV protease (TEVp), and treated with either 1 μM of A-1331852 or DMSO at the time of transfection. The addition of TEV protease or A-1331852 led to higher iADAR activity and mNeonGreen expression. 48 hours post transfection, cells were imaged and the mean mNeonGreen-to-mCherry ratio was computed for transfected cells (gated by mCherry expression) by ImageJ. Each dot represents a single cell (n=1 transfection).

FIG. 37A-37G show Sense Codon Editing for altering the localization, fusion state, and activity of an mRNA encoded protein (AGG to IGG). Sense codon editing of RNA regions encoding a 2A “skipping” peptide results in altered protein targeting, localization, and activity. FIG. 37A) Schematic of sense codon ADAR editing DNA construct. A dsRNA hairpin is inserted at the C-terminus of a skipping deficient T2A-G18R mutant with a MS2-C loop. In the presence of ADAR activity, the Gly18 is rescued by deamination of the AGG codon to IGG. FIG. 37B) Multiple-sequence alignment of various known 2A “self-cleaving” or “skipping” peptides. Sequences from different viruses are shown, including: P2A, porcine teschovirus-1 (SEQ ID NO: 360); T2A, thosea asigna virus (SEQ ID NO: 361); E2A, equine rhinitis A virus (SEQ ID NO: 362); F2A, foot-and-mouth disease virus (SEQ ID NO: 363). Conserved residues that are needed for the “skipping” activity of these 2A peptides are highlighted. Mutation of these residues eliminates self-cleavage/skipping activity, resulting in the translation of an unskipped (intact) fusion protein. FIG. 37C) Reporter design for editing-induced formation of a skipping peptide. In this design, iADAR activity is utilized to convert a mutated 2A sequence into a skipping active 2A peptide. In the absence of ADAR editing/prior to editing (left), an N-terminal secretion/signal-sequence targets the intact full-length protein into the ER, including the Gal4-VP64 transcription factor sequence. Upon ending of a target codon an active 2A peptide is generated, resulting in the skipping and release of a cytoplasmic Gal4-VP64, which can then be translocated to the nucleus to activate a target gene. Generation of the active 2A peptide is mediated by base editing of an arginine encoding send codon (AGG) to a sense codon that is interpreted as a glycine (IGG). IGG (right), which is read as a glycine, the Gal4-VP64 is now expressed in the cytoplasm where it can translocate into the nucleus and turn on an H2B-mCherry reporter that is integrated with upstream UAS elements. FIG. 37D) Active ADAR-editing of T2A(G18R) and release of Gal4-VP64 in HEK293FT-UAS-H2B-mCherry cells leads to an increase in the median H2B-mCherry fluorescence intensity. Cells were co-transfected with 3 ng of SS-Halo-T2A*-FLAG-Gal4-VP64 and 30 ng of MCP-ADAR or MCP-dADAR. FIG. 37E & FIG. 37F) Active ADAR-editing of T2A(G18R) and release of Gal4-VP64 in HEK293FT-UAS-H2B-mCherry cells leads to an increase in the population of H2B-mCherry positive cells. Cells were co-transfected with 0.3 ng (FIG. 37E) or 0.03 ng (FIG. 37F) of SS-Halo-T2A*-FLAG-Gal4-VP64 and 30 ng of MCP-ADAR or MCP-dADAR. Characteristic H2B-mcherry histograms of TagBFP-positive cells that are expressing inactive dADAR (black) or active ADAR (gray). The dotted lines represent the threshold for calling a cell mCherry-positive, determined by the top 0.5% of non-transfected cells. FIG. 37G) Western blot of HEK293FT-UAS-H2B-mCherry cells co-transfected with the T2A* construct and MCP-ADAR constructs stained for FLAG epitope and GAPDH loading control. Blank squares were transfected with filler DNA and the square with the d represents dADAR. Predicted masses of FLAG-fusion proteins: Halo-T2A*-FLAG-Gal4VP64—67 kDa (skipping incompetent) and FLAG-Gal4VP64—30 kDa (skipping competent). Additional potential bands due to incomplete skipping of C-terminal T2A-TagBFP: Halo-T2A*-FLAG-Gal4VP64-T2A-TagBFP—93 kDa and FLAG-Gal4VP64-T2A-TagBFP—56 kDa. FIG. 37D-37F) 24 hours post-transfection, cells were trypsinized and analyzed by flow cytometry. Transfected cells were identified based on gating for TagBFP (0.5% of non-transfected cells), and H2B-mCherry positive cells were identified based on gating for mCherry (0.5% of non-transfected cells). Values in FIG. 37D are plotted as median levels of H2B-mCherry in transfected cells. Values in FIG. 37E and FIG. 37F are plotted as percentage of mCherry-positive cells in transfected cells. The histograms shown in FIG. 37E and FIG. 37F are representative of T2A* transfected cells with MCP-ADAR or MCP-dADAR. Statistical analysis for significance performed via Prism: multiple student t-tests (n=3 separate transfections). ****—P<0.0001, ***—P<0.001, **—P<0.01, *—P<0.05.

FIG. 38A-38D show exemplary START-Codon Editing AUA to AUI nucleic acid Constructs (see e.g., FIG. 26), SEQ ID NOs: 292-295.

FIG. 39A-39C show exemplary START-Codon Editing AUA to AUI nucleic acid Constructs (signal sequence & HA), SEQ ID NOs: 296-298.

FIG. 40A-40B show exemplary START-Codon Editing AUG to AUI nucleic acid Constructs (see e.g., FIG. 27), SEQ ID NOs: 299-300.

FIG. 41 shows an exemplary In-Frame Protein Sequence Editing nucleic acid construct, see e.g., SEQ ID NO: 301.

FIG. 42A-42C show exemplary Two-Input AND-Gate with Multiple STOP codons nucleic acid constructs, SEQ ID NOs: 302-304.

FIG. 43 shows an exemplary nucleic acid construct with Internal STOP Codon Substrates, see e.g., SEQ ID NO: 305.

FIG. 44A-44G show exemplary nucleic acid constructs with Inclusion of ADAR Mutations and Localization Domain for Antigen Sensing, SEQ ID NOs: 306-313.

FIG. 45 shows an exemplary Multi-Input iADAR nucleic acid construct, SEQ ID NO: 314.

FIG. 46A-46C show exemplary Mutation of NS3-peptide based system nucleic acid constructs, SEQ ID NOs: 315-317.

FIG. 47A-47F show exemplary Ligand-Inducible Connection Based iADAR nucleic acid Constructs, SEQ ID NOs: 318-323.

FIG. 48A-48C show exemplary BAD-BclxL IRES nucleic acid Constructs, SEQ ID NOs: 324-326.

FIG. 49 shows an exemplary BAD-AD-BclxL IRES nucleic acid Construct, SEQ ID NO: 327.

FIG. 50 shows exemplary AUA to AUI (signal sequence & HA) amino acid Constructs, SEQ ID NOs: 328-329.

FIG. 51 shows an exemplary In-Frame Protein Sequence Editing amino acid Construct, SEQ ID NO: 330.

FIG. 52 shows an exemplary Internal STOP Codon Substrates amino acid Construct, SEQ ID NOs: 331-333.

FIG. 53A-53D show exemplary amino acid constructs with Inclusion of ADAR Mutations and Localization Domain for Antigen Sensing, SEQ ID NOs: 334-341.

FIG. 54 shows an exemplary Multi-Input iADAR Protein amino acid Construct, SEQ ID NO: 342.

FIG. 55A-55B show exemplary Mutation of NS3-peptide based system amino acid constructs, SEQ ID NOs: 343-345.

FIG. 56A-56C show exemplary Ligand-Inducible Connection Based iADAR amino acid Constructs, SEQ ID NOs: 346-351.

FIG. 57A-57B show exemplary BAD-BclxL IRES amino acid Constructs, SEQ ID NOs: 352-354.

FIG. 58 shows an exemplary BAD-AD-BclxL IRES amino acid Construct, SEQ ID NO: 355.

FIG. 59A-59B show exemplary In-Frame Protein Sequence Editing nucleic acid constructs, SEQ ID NOs: 356-357.

FIG. 60 shows exemplary In-Frame Protein Sequence Editing amino acid constructs, SEQ ID NOs: 358-359.

FIG. 61A-61C—In vitro transcribed iADAR sensors can be directly delivered to cells. FIG. 61A) Schematic of in vitro transcribed mRNA constructs. FIG. 61B) Schematic of iADAR mRNA that is delivered by lipofectamine and is dependent on drug to turn on fluorescent protein expression. FIG. 61C) HEK293FT cells either non-transfected (NT) or transfected with catalytically inactive ADAR (dADAR), constitutively active ADAR (ADAR), or conditionally active iADAR (BAD(V)) mRNA circuits. 48 hours post-transfection, cells were analyzed for expression of downstream mNeonGreen, as determined as having a value greater than 1% of non-transfected cells.

### DETAILED DESCRIPTION

Provided herein are compositions, kits, systems and methods related to an inducible adenosine deaminase acting on RNA (ADAR) enzyme, which can be activated in the presence of an inducer. These inducible ADAR (iADAR) enzymes can transition from an OFF (“iADAR-OFF”) to an ON (“iADAR-ON”) state in the presence of an inducer. When the iADAR is ON state, it can effect the translation of a gene of interest (GOI), depending on the nucleic acid construct that the iADAR acts on, resulting in translation of a GOI being turned ON or OFF. In some embodiments, an iADAR in the on state (iADAR-ON) can edit a target codon. As used herein the term “target codon” refers to a three base pair codon (e.g., a stop codon, a start codon, a non-start codon, or a sense codon) comprising at least one adenosine nucleotide in a double-stranded region of an RNA construct, which is targeted by the activated iADAR (iADAR “ON”), and the activated iADAR deaminates the at least one adenosine nucleotide in the target codon into an mosine nucleotide. By way of example only, an iADAR in the on state (iADAR-ON) can edit a STOP (UAG) codon to UIG, therefore eliminating the STOP codon.

In one embodiment, if the STOP codon is upstream (e.g., 5′) of a GOI (referred to herein as an “activation construct”), the iADAR-ON can remove the STOP codon resulting in translation of the downstream GOI. Thus, in this embodiment, in the presence of an inducer, gene translation is ON. That is—in the presence of the inducer, the translation of the GOI is switched from OFF→ON. In another embodiment, if the STOP codon is located 5′ of a GOI and a 3′ polyA signal (referred to herein as an “inactivation construct”), an iADAR-ON can edit and remove the STOP codon, resulting in translation of the polyA tail and leading to mRNA GOI decay. Thus, in this embodiment, in the presence of an inducer, gene translation is OFF. That is, in the presence of an inducer, the translation of the GOI is switched from ON→OFF. In some embodiments, the mRNA encoding the GOI is also destroyed by the cell.

As disclosed herein, the technology described herein relates to engineered human ADAR deaminase domains (DD), such that the ADAR is modified to be in a constitutively inactive state. Normally ADAR is constitutively active in the presence of its co-factor IP6. This engineering of the DDs of the ADAR enables the ADAR to be inducible, e.g., it is an engineered inducible ADAR (iADAR) that needs an inducer to turn it on. For iADAR to become activated i.e., to be turned ON, it is allosterically modulated from an inactive (iADAR-OFF) to an active state (iADAR-ON) in response to an inducer, e.g., without limitation, a small molecule drug, target antigen-binding, protease activity, and light, or any combination of these stimuli. For illustrative purposes only, and as disclosed herein, the pairing the engineered iADAR with a synthetic mRNA transcripts that comprise a target codon (e.g., STOP, START, non-START, or SENSE codon) located in a double-stranded region of the transcript (e.g., a ds-STOP, ds-START, or ds-SENSE region or loop) that localize the iADAR to an editable target codon enables the iADAR-ON to edit the target codon, therefore, in effect remove/eliminate or mutate the target codon, to change the protein expression of, or mRNA stability of a GOI in a synthetic construct. Accordingly, the technology disclosed herein enables the selective editing of target codons (e.g., STOP, START, non-START, or SENSE codons) in synthetic mRNA transcripts based on user defined and potentially endogenous inputs.

It is envisioned that the use of the iADAR as disclosed herein is not limited to acting on the synthetic constructs defined herein, rather, the inducible iADAR can be used in any gene editing method that uses an ADAR, including but not limited to gene therapy methods, such as, but not limited to, a viral or non-viral delivery of a nucleic acid to a subject that has a target codon (e.g., a STOP, START, non-START, or SENSE codon). Uses of an iADAR as disclosed herein in gene therapy applications enables a system for improved control and/or regulation of a GOI being delivered by the viral vector or non-viral vector, for example, for safety methods enabling GOI expression only when the inducer of the iADAR is present, and/or degradation of the GOI if the delivered GOI needed to be eliminated.

Moreover, in some embodiments, the iADAR can be used in any gene editing method where the target codon (e.g., STOP, START, non-START, or SENSE codon) is inserted into a target nucleic acid sequence, for example, using gene editing methodologies such as CRISPR systems.

I. iADAR

Deaminase proteins, such as, but not limited to, Adenosine Deaminase Acting on RNA (ADAR) have recently been developed as novel tools for RNA editing. In mammalian cells, there are three types of ADAR proteins, Adar1 (two isoforms, p110 and p150), Adar2 and Adar3 (catalytically inactive). The catalytic substrate of ADAR protein is double-stranded RNA, and it can remove the NH 2 group from an adenosine (A) nucleobase, changing A to inosine (I), which is recognized as guanosine (G) and paired with cytidine (C) during subsequent cellular transcription and translation processes. Previous modifications to ADAR have been reported, where λN peptide is fused to human Adar1 or Adar2 deaminase domain to construct the λN-ADARDD system, which could be guided to bind specific RNA targets by a fusion RNA consisting of BoxB stem loop and antisense RNA. Such a modified λN-ADARDD can edit and change a target A to I by introducing an A-C mismatch at the target A base, resulting in A to G RNA base editing. Other methods for RNA editing include fusing antisense RNA to R/G motif (ADAR-recruiting RNA scaffold) to edit target RNA by overexpressing Adar1 or Adar2 proteins in mammalian cells, and using dCas13-ADAR to precisely target and edit RNA. Additionally, reports of engineered RNA that is partially complementary to the target transcript to recruit native ADAR1 or ADAR2 to change adenosine to inosine at a specific site in a target RNA have been reported, and are referred to as “LEAPER” (Leveraging Endogenous ADAR for Programmable Editing on RNA) and the ADAR-recruiting RNAs are referred to interchangeably as “dRNA” or “arRNA”, which is disclosed in UA application 20210355494, which is incorporated herein in its entirety reference.

The technology disclosed herein is directed to an inducible ADAR (iADAR), where ADAR has been engineered to be active only in the presence of an inducer, and where the iADAR can edit target codons (e.g., stop, start, non-start, or sense codons) in a synthetic mRNA transcript comprising a ds-target codon (ds-TC) region as disclosed herein, wherein the ds-TC region comprises a target codon located in a double stranded region and a binding motif (BM) for a RNA binding domain, as disclosed herein. As an exemplary example, an iADAR that is ADAR2-DD(E488Q) that is fused to the C-terminus of bacteriophage-derived MS2 coat protein (MCP), which serves as a RNA binding domain and binds a specific RNA motif. While the fusion of MCP to ADAR has previously been reported to have editing activity on dsRNA duplex between a substrate strand and a guide strand, it was for targeting the adenosine deaminase activity to a specific RNA target sequence. Herein, the inventors further engineered and improved the ADAR in that the deaminase domain (DD) has been modified to be inducible, so that adenosine deaminase activity is only ON or functional in the presence of a specific inducer, enabling inducer-dependent adenosine deaminase activity, e.g., editing a target codon (e.g., STOP, START, non-START, or SENSE codon) present on the short hairpin motif in the presence of an inducer. More specifically, the inventors have modified the deaminase domain of the AR to include (i) a RNA binding domain (RBD) that binds to a specific binding motif on a ds-TC region (e.g., ds-STOP, ds-START, or ds-SENSE region) disclosed herein, and (ii) an affinity binding pair that activates the DD in the presence of an inducer, and (iii) specific modifications to the DD polypeptide that enables the function of the adenosine deaminase to be activated only by the presence of an inducer (i.e., the DD is modified to be constitutively active so that the affinity binding pair controls the adenosine deaminase activity) therefore resulting in an inducible ADAR that is capable of target codon (e.g., stop, start, non-start, or sense codon) editing on the same strand to a target transcript or GOI when it expressed in human cells.

In some embodiments, the iAD or iADAR comprises the amino acid sequences of one of SEQ ID NOs: 1-37, 88-94, 168, 169, or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 1-37, 88-94, 168, 169, that maintains the same function.

A. iADAR Fusion Protein Components

One aspect of the technology relates to an inducible ADAR (iADAR). In some embodiments, an iADAR is a fusion protein comprising, in brief, two deaminase domains (DD) or two portions of a deaminase domain, each deaminase domain, or portion thereof, attached to, or associated with, a binding protein of a binding pair, where each of the binding proteins bind to each other in the absence of an inducer. When the two binding proteins of the binding pair bind to each other (e.g., in the absence of an inducer), it deforms the IP6-binding pocket and sterically inhibits access of the cofactor IP6 to activate ADAR, therefore the iADAR is in the inactivated or OFF state (iADAR-OFF). Without wishing to be bound by theory, deformation of the IP6 binding pocket prevents stable/ordered IP6 binding and folding of the IP6 binding pocket. “Access” of IP6 to the binding site residues may be impeded (e.g., in a solvent, IP6 may transiently interact with a couple of residues). The coordination of the IP6 interacting residues into the active, folded state of ADAR is impaired. When the inducer is present, the binding pair no-longer bind to each other, enabling access of the IP6 to the binding site in the ADAR, and activating the iADAR to the ON state (i.e., iADAR-ON).

In particular embodiments, the fusion protein comprises: (a) a first portion of a deaminase domain (DD) of an adenosine deaminase; (b) a first member of a binding pair associated with the first portion of the DD; (c) a second portion of the DD; and (d) a second member of a binding pair associated with the second portion of the DD, wherein the first member of the binding pair binds to the second member of the binding pair in the absence of an inducer, resulting in allosteric inhibition of the first and second portions of the DD, and wherein the first member of the binding pair does not bind to the second member of the binding pair in the presence of the inducer, resulting in activation of the first and second portions of the DD.

In some embodiments, in the absence of an inducer, the first and second portions of the DD allosterically inhibit the IP6 binding pocket, e.g., by deformation of the inositol hexaphosphate (IP6) binding pocket, and/or by preventing access of IP6 to a IP6 binding pocket.

In some embodiments, the iADAR fusion protein is a modified adenosine deaminase (AD) selected from any of: Adenosine Deaminase Acting on RNA (ADAR), Adenosine Deaminase TRNA Specific (ADAT), or Adenosine Deaminase Domain Containing (ADAD).

In exemplary embodiments, the iADAR is an engineered Adenosine Deaminase Acting on RNA (ADAR) fusion protein. In some embodiments, the iADAR is selected from an engineered ADAR1, ADAR2, or ADAR3 molecule. In some embodiments, the iADAR is an engineered ADAR1 polypeptide or engineered ADAR2 polypeptide.

In certain embodiments, the iADAR is engineered from a natural or endogenously ADAR present in the host cell, for example, naturally or endogenously present in the eukaryotic cell. In some embodiments, the iADAR is modified based on an iADAR that is endogenously expressed by the host cell. In certain embodiments, the iADAR is exogenous to the host cell. In some embodiments, the iADAR is encoded by a nucleic acid (e.g., DNA or RNA) as disclosed herein. In some embodiments, the method comprises introducing the iADAR or a nucleic acid construct encoding the iADAR into the host cell. In some embodiments, the method does not comprise introducing any protein into the host cell. In some embodiments, the method comprises delivery of ribonucleoprotein comprising an RNA molecule as described herein. In some embodiments, the method comprises co-delivery of the iADAR (or nucleic encoding it) and an RNA molecule as described herein. In certain embodiments, the iADAR is iADAR1 and/or iADAR 2. In some embodiments, the iADAR is one or more iADARs selected from the group consisting of hiADAR1, hiADAR2, murine iADAR1 and murine iADAR2.

In some embodiments, an iADAR2 fusion protein comprises the DD of ADAR2 and comprises residues of SEQ ID NO: 95, or a polypeptide having at least about 85%, or about 85%, or about 90%, or about 95%, or about 98% homology to SEQ ID NO: 95, where SEQ ID NO: 95 comprises E488Q modification for a constitutively active deaminase activity of ADAR2. The E488Q modification of ADAR2 increases the catalytic efficiency and rate of the enzyme as compared to the non-modified ADAR2 enzyme, which is also constitutively active. In some embodiments the iADAR2 can comprise a single polypeptide, or be split into two DD portions, e.g., a DDN and DDC fragments as disclosed herein.

RNA-Binding Domain of the iADAR

In some embodiments, the iADAR fusion protein comprises a RNA-binding domain (RBD) that binds to a binding motif for RBD, which in some embodiments, is located in the ds-TC region (e.g., ds-STOP, ds-START, or ds-SENSE region) of an RNA molecule.

In some embodiments, the RNA-binding domain is selected from the group consisting of MCP, PCP, λN, and HIV tat. In some embodiments, the RNA-binding domain comprises MCP which binds to the Binding motif for RBD (BM) that comprises MS2. In some embodiments, the RNA-binding domain comprises tandem dimers of MCP (tdMCP), which bind to the Binding motif for RBD (BM) that comprises MS2. In some embodiments, the RNA-binding domain comprises PCP which binds to the Binding motif for RBD (BM) that comprises PP7. In some embodiments, the RNA-binding domain comprises tandem dimers of PCP (tdPCP), which bind to the Binding motif for RBD (BM) that comprises PP7. In some embodiments, the RNA-binding domain comprises λN which binds to a ds-TC region (e.g., ds-STOP, ds-START, or ds-SENSE region) that comprises the Binding motif for RBD (BM) that comprises BoxB. In some embodiments, the RNA-binding domain comprises HIV Tat, which binds to a ds-TC region (e.g., ds-STOP, ds-START, or ds-SENSE region) that comprises the Binding motif for RBD (BM) that comprises TAR.

In some embodiments, the RNA-binding domain (RBD) is MCP having an amino acid sequence comprising:

(SEQ ID NO: 100)
MASNFTQFVLVDNGGTGDVTVAPSNFANGIAEWISSNSRSQAYKVTCSV
RQSSAQNRKYTIKVEVPKGAWRSYLNMELTIPIFATNSDCELIVKAMQG
LLKDGNPIPSAIAANSGIY.

In some embodiments of any of the aspects, the RNA-binding domain is MCP that comprises an amino acid of SEQ ID NO: 100. In some embodiments of any of the aspects, the sequence of the RNA-binding domain is MCP comprising SEQ ID NO: 100 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 100 that maintains the same functions as SEQ ID NO: 100 (e.g., where the RNA-binding domain MCP binds to the Binding motif for RBD (BM) MS2).

In some embodiments, the RNA-binding domain (RBD) is PCP having an amino acid sequence comprising:

(SEQ ID NO: 101)
MSKTIVLSVGEATRTLTEIQSTADRQIFEEKVGPLVGRLRLTASLRQNG
AKTAYRVNLKLDQADVVDSGLPKVRYTQVWSHDVTIVANSTEASRKSLY
DLTKSLVATSQVEDLVVNLVPLG.

In some embodiments of any of the aspects, the RNA-binding domain is PCP that comprises an amino acid of SEQ ID NO: 101. In some embodiments of any of the aspects, the sequence of the RNA-binding domain is PCP comprising SEQ ID NO: 101 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 101 that maintains the same functions as SEQ ID NO: 101 (e.g., where the RNA-binding domain PCP binds to the Binding motif for RBD (BM) PP7).

In some embodiments, the RNA-binding domain (RBD) is a mutated RBD, e.g., as disclosed in U.S. Provisional application 63/578,836, filed Aug. 25, 2023, the contents of which are incorporated herein by reference in its entirety. The mutated RBD can be derived from MCP or PCP to create a destabilized MCP or PCP. In some embodiments, the destabilized MCP or PCP comprises at least one degron domain that leads to degradation of any polypeptide comprising it when the polypeptide is not bound to its cognate binding motif in the RNA.

In some embodiments, the RNA-binding domain (RBD) is λN having an amino acid sequence comprising: MADAQTRRRERRAEKQAQWKAAN (SEQ ID NO: 102). In some embodiments of any of the aspects, the RNA-binding domain is λN that comprises an amino acid of SEQ ID NO: 102. In some embodiments of any of the aspects, the sequence of the RNA-binding domain is λN comprising SEQ ID NO: 102 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 102 that maintains the same functions as SEQ ID NO: 102 (e.g., where the RNA-binding domain λN binds to the Binding motif for RBD (BM) BoxB).

In some embodiments, the RNA-binding domain (RBD) is HIV tat having an amino acid sequence comprising: MASGPRPRGTRGKGRRIRR (SEQ ID NO: 103). In some embodiments of any of the aspects, the RNA-binding domain is HIV tat that comprises an amino acid of SEQ ID NO: 103. In some embodiments of any of the aspects, the sequence of the RNA-binding domain is HIV tat comprising SEQ ID NO: 103 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 103 that maintains the same functions as SEQ ID NO: 103 (e.g., where the RNA-binding domain HIV tat binds to the Binding motif for RBD (BM) HIV TAR).

In some embodiments the RBD is located at the N-terminal of the iAD fusion protein. In some embodiments, the iAD fusion protein comprises a RBD attached to the C-terminus of a DD, or DDN, as disclosed herein. In some embodiments, there is a linker located between the RBD and the DD or DDN. In some embodiments, the RBD is located at the most C-terminal end of the iAD fusion protein. In some embodiments, the RBD is located at an internal position of the iAD fusion protein.

### B. Deaminase Domain (DD) of Adenosine Deaminase (AD)

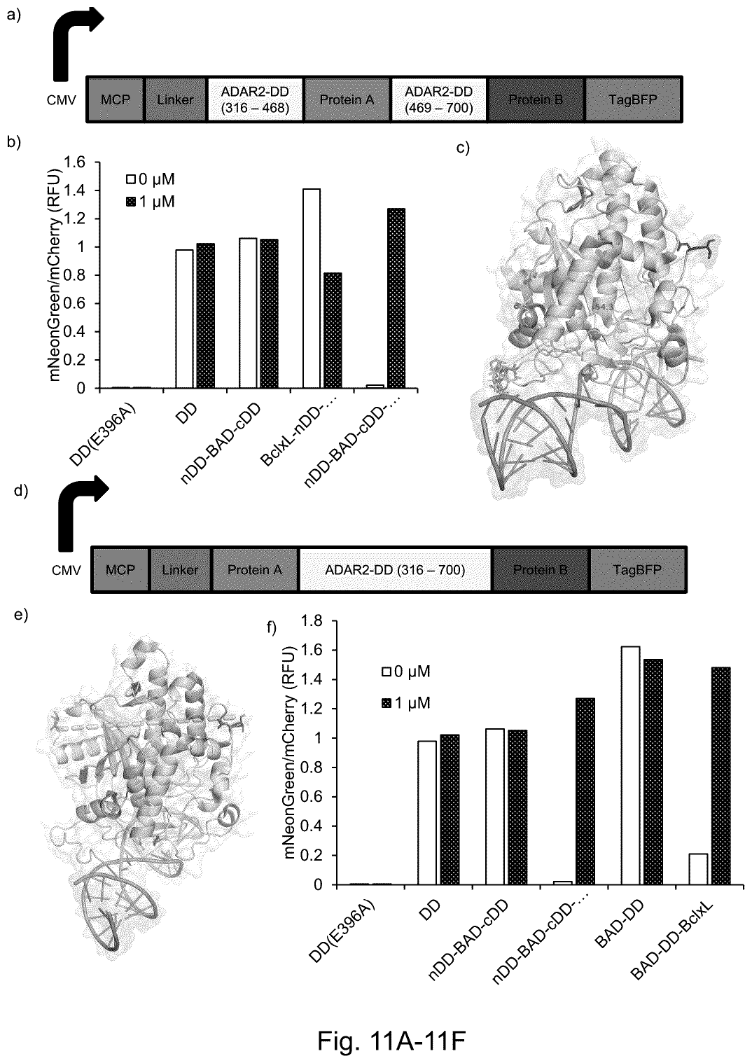

Without wishing to be bound by theory, the deaminase domain (DD) of an adenosine deaminase enzyme as disclosed herein, e.g., ADAR, ADAT, or ADAD, can be fused to 2 members of a binding pair, where the binding pair bind to each other in the absence of an inducer, preventing the activation of the AD enzyme, as disclosed herein. In some embodiments each member of the binding pair binds to different portions of the DD, there the DD is a single polypeptide, e.g., for example, see FIG. 11E. In alternative embodiments, the DD is split into two fragments or portions, where the two fragments of the DD form a heterodimer, and each member of the binding pair is fused to each of the DD-split fragments, for example, see FIG. 11A.

In some embodiments, the iADAR comprises a deaminase domain of adenosine deaminase (referred to herein as “AD-DD” or “AD-deaminase domain”) that is split into at least two fragments; (i) a first portion of the deaminase domain (AD-DDN or nDD) and (ii) a second portion of a deaminase domain (AD-DDC or cDD). Stated differently, the deaminase domain of the adenosine deaminase (AR-DD), such as but not limited to ADAR, is split into two fragments or polypeptide portions, referred to herein as AD-DDN and AD-DDC, referring to a N-terminal portion of the DD and a C-terminal portion of the DD, respectively. In some embodiments, the two polypeptide portions of DD (i.e., AD-DDN and AD-DDC), together have deaminase activity—that is, both the AD-DDN and AD-DDC are required for deaminase activity. In some embodiments, the AD-DDN has adenosine deaminase activity that is blocked or inhibited by the binding pair, BP1 and BP2 in the absence of an inducer. In some embodiments, the AD-DDC has adenosine deaminase activity that is blocked or inhibited by the binding pair, BP1 and BP2 in the absence of an inducer.

In some embodiments, the two polypeptide fragments of the deaminase domain (e.g., AD-DDN and AD-DDC) of the iADAR fusion protein are capable of converting at least one stop codon into at least one non-stop codon. In some embodiments, the AD-deaminase domain (that is split into two polypeptide fragments; AD-DDN and AD-DDC) is modified so that the adenosine deaminase is constitutively active—that is, if the binding protein pair was not associated, the AD-deaminase domain would be constitutively active (however, as it is part of the iADAR, the adenosine deaminase activity is inhibited in the absence of an inducer). In some embodiments, the AD-deaminase domain, which is split into 2 or more fragments, is a constitutively active AD-deaminase domain, and can, for example comprise one of: an E1008Q mutation in ADAR1; an E488Q mutation in ADAR2; or an E527Q mutation in ADAR3. The E1008Q, E488Q, and E527Q modifications of ADAR1, ADAR2, and ADAR3, respectively, increase the catalytic efficiency and rate of the enzyme as compared to the non-modified enzyme, which is also constitutively active.

In some embodiments, the AD-deaminase domain is from Adenosine Deaminase TRNA Specific (ADAT), for example, ADAT1. In some embodiments, the AD-deaminase domain is from Adenosine Deaminase Domain Containing (ADAD), for example, but not limited to, ADAD1 or ADAD2. In some embodiments, the AD-deaminase domain is from ADAR, ADAT, or ADAD that is a mammalian adenosine deaminase. In some embodiments, the ADAR, ADAT, or ADAD is a human adenosine deaminase.

In some embodiments, the iADAR as disclosed herein comprises a AR-deaminase domain that is split into two or more fragments at the location of a RNA binding loop, e.g., wherein the RNA binding loop is the 5′ RNA binding loop (RBL) of ADAR1, ADAR2, ADAR3, ADAD1, or ADAD2. In some embodiments, the AD-deaminase domain is split into two polypeptide fragments (e.g., AD-DDN and AD-DDC) at a 5′ RNA binding loop (RBL), where the RBL is selected from any of: residues G969 to K999 of ADAR1: GALFDKSCSDRAMESTESRHYPVFENPKQGK (SEQ ID NO: 134) of ADAR1; residues A454 to Q479 of ADAR2: ARIFSPHEPILEEPADRHPNRKARGQ (SEQ ID NO: 135); residues A493 to H518 of ADAR3: ARLHSPYEITTDLHSSKHLVRKFRGH (SEQ ID NO: 136); residues A334 to K365 of ADAD1: AQIKSQLRLNPHSISAFEANEELCLHVAVEGK (SEQ ID NO: 137); residues A347 to Q375 of ADAD2: AARDIYLPPTSEGGLPHSPPMRLQAHVLGQ (SEQ ID NO: 138); residues K974 to S986 of ADAR1: KSCSDRAMES (SEQ ID NO: 139) of ADAR1; residues F457 to D469 of ADAR2: FSPHEPILEEPAD (SEQ ID NO: 140); residues P498 to S508 of ADAR3: PYEITTDLHSS (SEQ ID NO: 141); residues Q339 to P344 of ADAD1: QLRLNP (SEQ ID NO: 142); or residues P352 to P360 of ADAD2: PPTSEGGLP (SEQ ID NO: 143).

In some embodiments, where the iADAR comprises a AD-deaminase domain from ADAR1, the AD-deaminase domain is split between any of the following: residues S977 and D978 of ADAR1 or residues T984 and E985 of ADAR1, or residues L340 and R341 of ADAD1. In some embodiments, where the iADAR comprises a AD-deaminase domain from ADAR2, the AD-deaminase domain is split between residues A468 and D469 of ADAR2 residues G357 and G358 of ADAD2. In some embodiments, where the iADAR comprises a AD-deaminase domain from ADAR3, the AD-deaminase domain is split between residues S507 and S508 of ADAR3.

In some embodiments, where the DD is a single polypeptide, it comprises amino acids 316-700 of ADAR2, for example, it comprises the amino acid residues of the following sequence:

(SEQ ID NO: 95)
QLHLPQVLADAVSRLVLGKFGDLTDNFSSPHARRKVLAGVVMTTGTDVK
DAKVISVSTGTKCINGEYMSDRGLALNDCHAEIISRRSLLRFLYTQLEL
YLNNKDDQKRSIFQKSERGGFRLKENVQFHLYISTSPCGDARIFSPHEP
ILEEPADRHPNRKARGQLRTKIESGQGTIPVRSNASIQTWDGVLQGERL
LTMSCSDKIARWNVVGIQGSLLSIFVEPIYFSSIILGSLYHGDHLSRAM
YQRISNIEDLPPLYTLNKPLLSGISNAEARQPGKAPNFSVNWTVGDSAI
EVINATTGKDELGRASRLCKHALYCRWMRVHGKVPSHLLRSKITKPNVY
HESKLAAKEYQAAKARLFTAFIKAGLGAWVEKPTEQDQFSLT,

where E488Q is present.

In some embodiments of any of the aspects, the DD of ADAR2 comprises an amino acid of SEQ ID NO: 95. In some embodiments of any of the aspects, the sequence of the DD of ADAR2 comprises SEQ ID NO: 95 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 95 that maintains the same functions as SEQ ID NO: 95 (e.g., ADAR2-DD that comprises the E488Q modification).

In some embodiments of the aspects, the sequence of the DD of ADAR 2 (e.g., ADAR2-DDC) comprises SEQ ID NO: 95 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 95 and comprises the E448Q modification (i.e., which correspond to a Q residue at position 173 in SEQ ID NO: 95), and that maintains the same functions as SEQ ID NO: 95 (e.g., ADAR2-DD, which comprises the E488Q modifications).

In some embodiments, where the DD of ADAR2 is a single polypeptide, it comprises amino acids 316-700 of ADAR2, for example, it comprises the amino acid residues of the following sequence:

(SEQ ID NO: 96)
QLHLPQVLADAVSRLVLGKFGDLTDNFSSPHARRKVLAGVVMTTG
TDVKDAKVISVSTGTKCINGEYMSDRGLALNDCHAAIISRRSLLR
FLYTQLELYLNNKDDQKRSIFQKSERGGFRLKENVQFHLYISTSP
CGDARIFSPHEPILEEPADRHPNRKARGQLRTKIESGQGTIPVRS
NASIQTWDGVLQGERLLTMSCSDKIARWNVVGIQGSLLSIFVEPI
YFSSIILGSLYHGDHLSRAMYQRISNIEDLPPLYTLNKPLLSGIS
NAEARQPGKAPNFSVNWTVGDSAIEVINATTGKDELGRASRLCKH
ALYCRWMRVHGKVPSHLLRSKITKPNVYHESKLAAKEYQAAKARL
FTAFIKAGLGAWVEKPTEQDQFSLT,

where E396A and E488Q modifications are present.

In some embodiments of any of the aspects, the DD of ADAR2 comprises an amino acid of SEQ ID NO: 96. In some embodiments of any of the aspects, the sequence of the DD of ADAR2 comprises SEQ ID NO: 96 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 96 that maintains the same functions as SEQ ID NO: 96 (e.g., ADAR2-DD that comprises E396A and E488Q modification).

In some embodiments of the aspects, the sequence of the DD of ADAR 2 (e.g., ADAR2-DDC) comprises SEQ ID NO: 96 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 96 and comprises E396A and E448Q modifications (i.e., which correspond to an A residue at position 81 in SEQ ID NO: 96 (E96A) a Q residue at position 173 in SEQ ID NO: 96), and that maintains the same functions as SEQ ID NO: 96 (e.g., ADAR2-DD, which comprises both E396A and E488Q modifications).

In some embodiments, where the DD of ADAR2 is split into two portions, for example (i) a N-terminal portion (ADAR2-DDN) and (ii) a C-terminal portion (ADAR2-DDC), the N-terminal portion comprises amino acids 316-486 of ADAR2. In some embodiments, the C-terminal portion of the AD-DD of ADAR2 comprises amino acids 469-700 of ADAR2.

In some embodiments, ADAR2-DDN comprises the amino acids of

(SEQ ID NO: 97)
QLHLPQVLADAVSRLVLGKFGDLTDNFSSPHARRKVLAGVVMTTG
TDVKDAKVISVSTGTKCINGEYMSDRGLALNDCHAEIISRRSLLR
FLYTQLELYLNNKDDQKRSIFQKSERGGFRLKENVQFHLYISTSP
CGDARIFSPHEPILEEPA.

In some embodiments of any of the aspects, the ADAR2-DDN comprises an amino acid of SEQ ID NO: 97. In some embodiments of any of the aspects, the sequence of the ADAR2-DDN comprises SEQ ID NO: 97 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 97 that maintains the same functions as SEQ ID NO: 97 (e.g., ADAR2-DDN).

In some embodiments, the ADAR-DDC comprises the amino acids of:

(SEQ ID NO: 98)
DRHPNRKARGQLRTKIESGQGTIPVRSNASIQTWDGVLQGERLLT
MSCSDKIARWNVVGIQGSLLSIFVEPIYFSSIILGSLYHGDHLSR
AMYQRISNIEDLPPLYTLNKPLLSGISNAEARQPGKAPNFSVNWT
VGDSAIEVINATTGKDELGRASRLCKHALYCRWMRVHGKVPSHLL
RSKITKPNVYHESKLAAKEYQAAKARLFTAFIKAGLGAWVEKPTE
QDQFSLT,

where E488Q is present. In some embodiments of any of the aspects, the ADAR2-DDC comprises an amino acid of SEQ ID NO: 98. In some embodiments of any of the aspects, the sequence of the ADAR2-DDC comprises SEQ ID NO: 98 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 98 that maintains the same functions as SEQ ID NO: 98 (e.g., ADAR2-DDC, which comprises E488Q modification).

In some embodiments of any of the aspects, the sequence of the ADAR2-DDC comprises SEQ ID NO: 98 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 98 and comprises E448Q modification (i.e., which corresponds to a Q residue at position 20 in SEQ ID NO: 98), and that maintains the same functions as SEQ ID NO: 98 (e.g., ADAR2-DDC, which comprises E488Q modification).

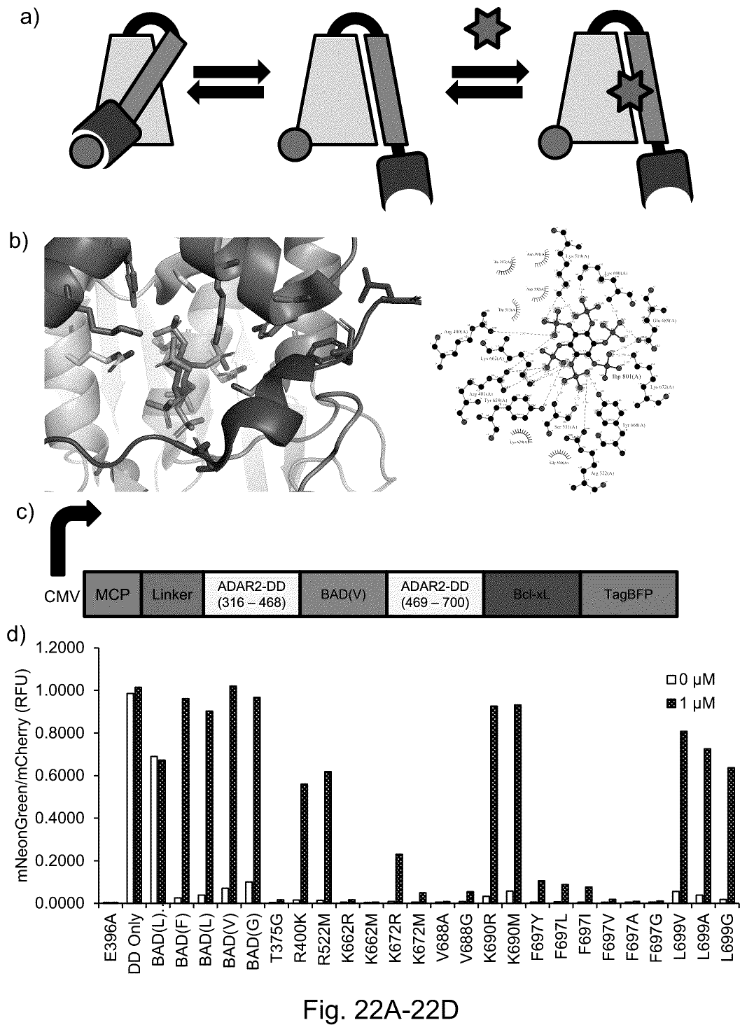

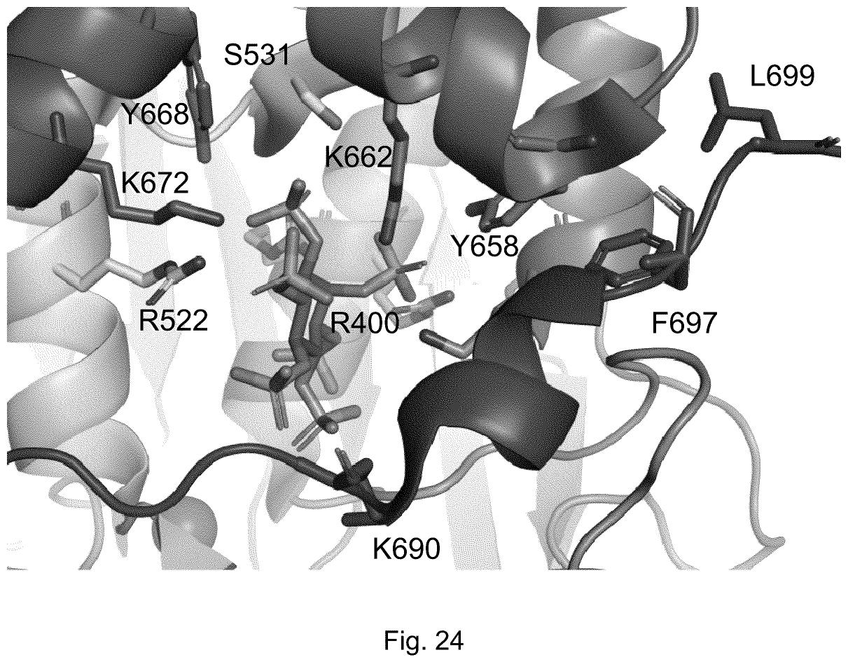

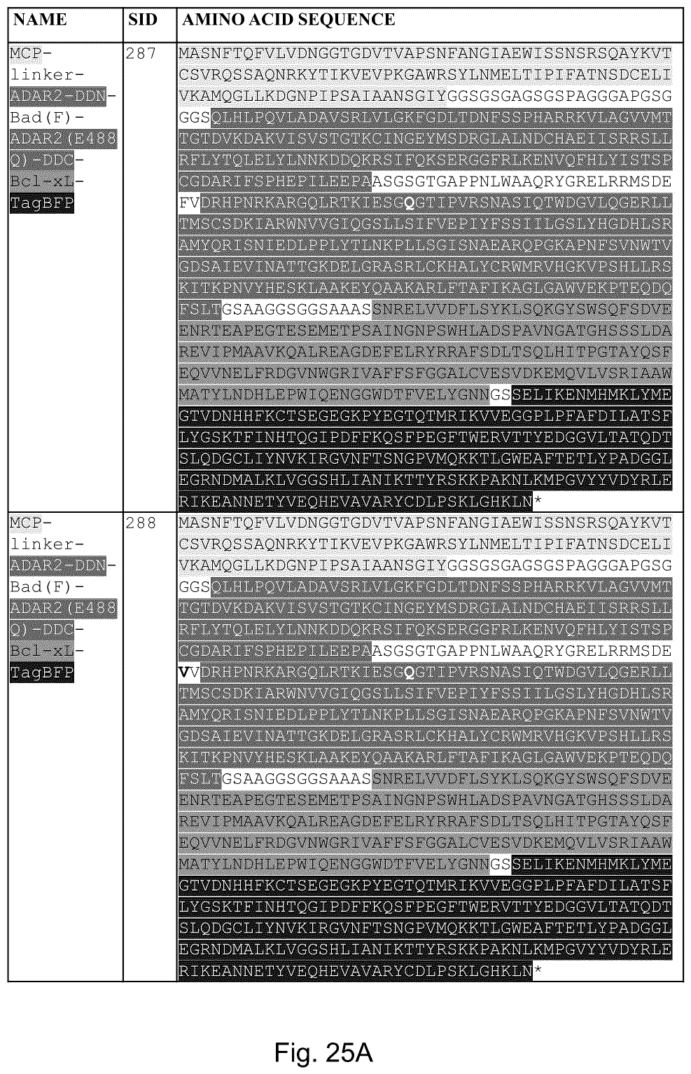

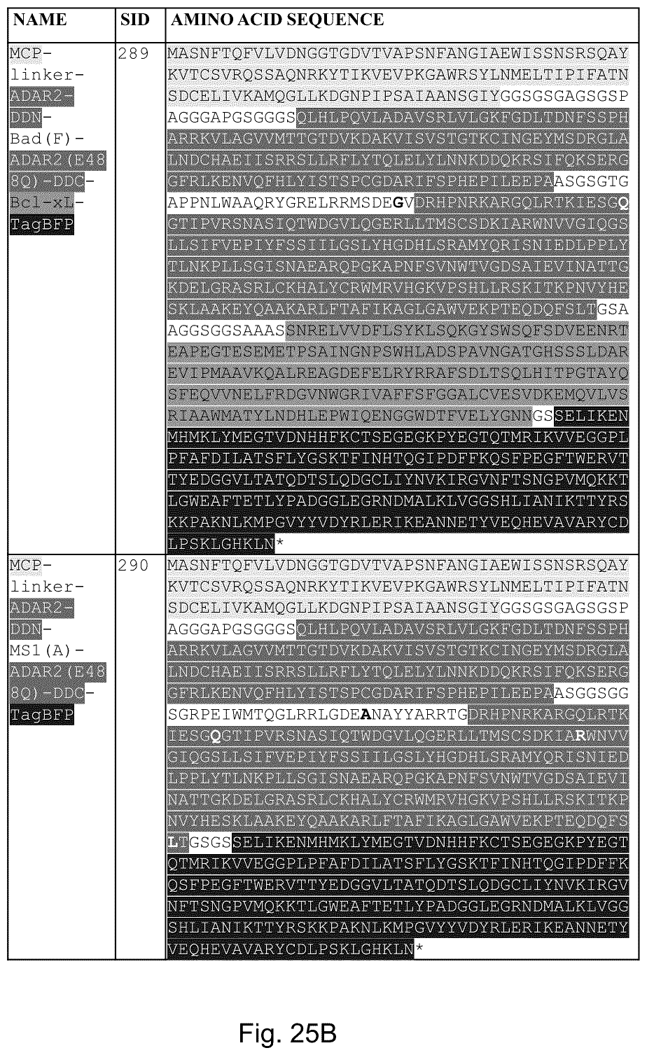

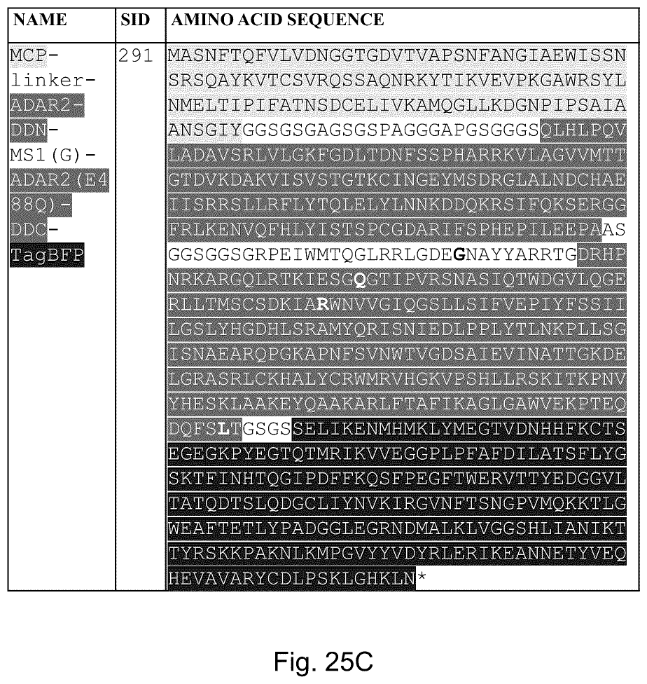

In some embodiments, the AD-deaminase domain (e.g., ADAR) comprises at least one mutation that decreases the background activity of the enzyme (e.g., activity on non-target RNAs; e.g., off-target activity). In some embodiments, the at least one mutation in the AD-deaminase domain (e.g., ADAR) is in the IP6 binding pocket (see e.g., FIG. 22-24, Example 12). In some embodiments, the at least one mutation in the AD-deaminase domain (e.g., ADAR; e.g., SEQ ID NO: 80) is in an amino acid residue selected from the group consisting of: R400, R522, S531, Y658, K662, Y668, K672, K690, F697, and L699 (see e.g., FIG. 24). In some embodiments, the at least one mutation in the AD-deaminase domain (e.g., ADAR; e.g., SEQ ID NO: 80) is in an amino acid residue selected from the group consisting of: T375, R400, R522, Y658, K662, Y668, K672, K690, F697, and L699. In some embodiments, the at least one mutation in the AD-deaminase domain (e.g., ADAR; e.g., SEQ ID NO: 80) is in an amino acid residue selected from the group consisting of: T375, R400, R522, K662, K672, V688, K690, F697, and L699 (see e.g., Table 11). In some embodiments, the at least one mutation in the AD-deaminase domain (e.g., ADAR; e.g., SEQ ID NO: 80) is selected from the group consisting of: T375G, R400K, R522M, K662R, K662M, K672R, K672M, V688A, V688G, K690R, K690M, F697Y, F697L, F6971, F697V, F697A, F697G, L699V, L699A, and L699G. In some embodiments, the at least one mutation in the AD-deaminase domain (e.g., ADAR; e.g., SEQ ID NO: 80) is selected from the group consisting of: R400K, R522M, K690R, and L699G. In some embodiments, the at least one mutation in the AD-deaminase domain (e.g., ADAR; e.g., SEQ ID NO: 80) is R522M and/or L699G.

In some embodiments, the fusion protein comprising at least one mutation in the AD-deaminase domain comprises one of SEQ ID NOs: 287-291 or an amino acid sequence that is at least at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 287-291, that maintains the same function.

### C. Affinity Binding Pairs

As disclosed herein, the first and second binding proteins of an affinity binding pair bind or interact, in the absence of an inducer, to allosterically and/or sterically inhibit the activation of the iADAR by the IP6 co-factor, therefore the iADAR is in the OFF state (iADAR-OFF). In some embodiments, in the absence of an inducer, the binding protein pair allosteric inhibit of the first and second portions of the DD (deaminase domain), where the inhibition is any one or more of: deformation of the inositol hexaphosphate (IP6) binding pocket of first and second portions of the DD, preventing access of IP6 to a IP6 binding pocket.

The affinity binding pair can comprise of two protein or polypeptides that, in the absence of an inducer, engage in protein-protein interaction with each other that link the first and the second portion of the DD together. For example, the fusion protein can comprise a first portion of a DD (AD-DDN) associated with a binding protein 1 (BP1), where the BP1 associates with BP2, where BP2 is fused to the second portion of the DD (AD-DDC). That is, in the absence of an inducer, the fusion protein comprises, not in any particular order, [AD-DDN]-[BP1]-[BP2]-[AD-DDC], where BP1 and BP2 binding pair link AD-DDN and AD-DDC and prevent IP6 access to the binding pocket. When an inducer is present, the AD-DDN and AD-DDC no longer interact and/or prevent IP6 from accessing the binding pocket and AD-DDN and AD-DDC together, have deaminase activity.

In some embodiments, BP1 and BP2 are switched positions, such that BP2 is associated with the first portion of the DD, and BP1 is associated with the second portion of the DD.

In some embodiments, the binding pair can be any linkage protein pairs or moieties that reversibly interact. As disclosed herein, there are different classes of binding pairs that can be used, for example but not limited to, simple ligand and ligand binding protein pair, antibody or antigen binding domain and peptide antigen, a repressible protease activation domain, a Degron domain, a induced-degradation domain, a induced-proximity domains, or a cytosolic sequestering domains, e.g., as disclosed in U.S. Pat. No. 11,530,246, which is incorporated herein in its entirety by reference.

In some embodiments, the first and second members of the binding pair are Bad and Bcl-xL, and the inducer of the first and second binding pairs is A-1331852. In some embodiments, the first and second members of the binding pair are Bad and Bcl-xL, and the inducer of the first and second binding pairs is ABT-737. In alternative embodiments, the first and second members of the binding pair are Bim and Bcl-xL, and the inducer of the first and second binding pairs is A-1331852. In alternative embodiments, the first and second members of the binding pair are Bim and Bcl-xL, and the inducer of the first and second binding pairs is ABT-737. In some embodiments, the first and second members of the binding pair are MS1 and MCL-1, and the inducer of the first and second binding pairs is S63845.

In alternative embodiments, the first member (BP1) of the binding pair comprises an antigen-binding domain, and the second member (BP2) of the binding pair comprises a first antigen, and the inducer comprises a second antigen, where the antigen-binding domain binds to the second antigen with a higher affinity than to the first antigen. That is, the inducer functions as a competitive inhibitor, and BP1 binds with greater affinity to the inducer than to the second antigen of BP2, thereby disrupting the interaction between BP1 and BP2.

In alternative embodiments, the first member (BP1) of the binding pair comprises an antigen-binding domain, and the second member (BP2) of the binding pair comprises a first antigen, and the inducer comprises a second antigen, where the antigen-binding domain binds to the second antigen with a similar affinity than to the first antigen. That is, the inducer functions as a competitive inhibitor, and BP1 binds with similar affinity to the inducer than to the second antigen of BP2, thereby disrupting the interaction between BP1 and BP2.

In some embodiments, the first member (BP1) of the binding pair comprises an anti-ALFA antigen binding domain, and the second member (BP2) of the binding pair comprises a first ALFA antigen, and the inducer of the first and second binding pairs comprises a second ALFA antigen, where anti-ALFA antigen binding domain binds to the second ALFA antigen with a higher affinity than to the first ALFA antigen.

In some embodiments, the first member (BP1) of the binding pair comprises an anti-ALFA antigen binding domain, and the second member (BP2) of the binding pair comprises a first ALFA antigen, and the inducer of the first and second binding pairs comprises a second ALFA antigen, where anti-ALFA antigen binding domain binds to the second ALFA antigen with a similar affinity compared to the first ALFA antigen.

In alternative embodiments, there is a cleavable linkage located either between the AD-DDN and BP1 or AD-DDC and BP2. That is, in the absence of the inducer, the BP1 and BP2 interact preventing activation of iADAR. When the inducer is present, it can cleave and separate either AD-DDN from BP1 and/or AD-DDC from BP2, therefore while BP1 and BP2 may still interact they no longer sterically inhibit access of IP6 to the binding pocket.

Accordingly, in one embodiment, the iADAR comprises a first member of the binding pair (BP1) that is associated with the AD-DDN, and a second member of the binding pair (BP2) that is associated with the AD-DDC, and where there is a cleavable linker located between the BP2 and AD-DDC. In such an embodiment, in the absence of an inducer, the BP2 is associated with the AD-DDC, therefore the BP1 and BP2 members of the binding pair interact and sterically hinder access of the IP6 cofactor to its binding site (therefore the iADAR is in the iADAR-OFF state or configuration). In the presence of an inducer that is a protease that specifically cleaves the cleavable linker, the association between BP2 and AD-DDC is broken, therefore removing or preventing the steric hindrance by the binding pair, therefore resulting in the iADAR in the iADAR-ON state.

In some embodiments, the cleavable linker is cleaved by an inducer that is a signal, e.g., light signal or sound signal. In some embodiments, the cleavable linker is cleaved by a protease or enzymatic cleavage signal, and the inducer is a protease.

TABLE 4A
List of Exemplary Binding pairs, with
the Binding Protein 1 (BP1) and
cognate Binding Protein 2 (BP2)

| BP1 | BP2 |
|---|---|
| Bim ((SEQ ID NO: 118) | Bcl-XL (SEQ ID NO: 117) |
| MS1(I)(SEQ ID NO: 119) | Mcl-1 (SEQ ID NO: 120) |
| Bad(L) (SEQ ID NO: 121) or | Bcl-XL (SEQ ID NO: 117) |
| BAD (SEQ ID NO: 116) or | |
| Bad(F) ((SEQ ID NO: 115) | |
| ALFA (SEQ ID NO: 122) or | NbALFA (SEQ ID NO: 123) |
| ALFA-PE ((SEQ ID NO: 124) or | |
| ALFA-78 ((SEQ ID NO: 125) | |
| SpyTag (SEQ ID NO: 126) | SpyCatcher (SEQ ID NO: 127) |

TABLE 4B
Exemplary Affinity Binding pairs are selected from any of:

| SEQ | Binding Pair | |
|---|---|---|
| ID NO: | (BP1) | Sequence |
| 115 | Bad(F) | ASGSGTGAPPNLWAAQRYGRELRRMSDEFV |
| 116 | Bad | APPNLWAAQRYGRELRRMSDEFVDSFKK |
| 117 | Bcl-XL | SNRELVVDFLSYKLSQKGYSWSQFSDVEENRTEAPEGTESEMETPS |
| | | AINGNPSWHLADSPAVNGATGHSSSLDAREVIPMAAVKQALREAGD |
| | | EFELRYRRAFSDLTSQLHITPGTAYQSFEQVVNELFRDGVNWGRIV |
| | | AFFSFGGALCVESVDKEMQVLVSRIAAWMATYLNDHLEPWIQENGG |
| | | WDTFVELYGNN |
| 118 | Bim | ASGSGSGDMRPEIWIAQELRRIGDEFNAYYARRTG |
| 119 | MS(1)A | ASGGSGGSGRPEIWMTQGLRRLGDEANAYYARRTG |
| 120 | Mcl1 | DELYRQSLEIISRYLREQATGAKDTKPMGRSGATSRKALETLRRVG |
| | | DGVQRNHETAFQGMLRKLDIKNEDDVKSLSRVMIHVFSDGVTNWGR |
| | | IVTLISFGAFVAKHLKTINQESCIEPLAESITDVLVRTKRDWLVKQ |
| | | RGWDGFVEFFHVEDLEGG |
| 121 | Bad(L) | ASGSGTGAPPNLWAAQRYGRELRRMSDELV |
| 122 | ALFA | SRLEEELRRRLTEP |
| 123 | NbALFA | EVQLQESGGGLVQPGGSLRLSCTASGVTISALNAMAMGWYRQAPGE |
| | | RRVMVAAVSERGNAMYRESVQGRFTVTRDFTNKMVSLQMDNLKPE |
| | | DTAVYYCHVLEDRVDSFHDYWGQGTQVTVSS |
| 124 | ALFA-PE | GSGPGRLEEELRRRLSPG |
| 125 | ALFA-78 | ASGSGPGRLEQEIRARLSPGT |
| 126 | Spy Tag | ASGGSGAHIVMVDAYKPTKGTG |
| 127 | SpyCatcher | MVDTLSGLSSEQGQSGDMTIEEDSATHIKFSKRDEDGKELAGATME |
| | | LRDSSGKTISTWISDGQVKDFYLYPGKYTFVETAAPDGYEVATAIT |
| | | FTVNEQGQVTVNGKATKGDAHIG* |
| 128 | PoC1 | VIPDYFKQSFPEGYSWERSMTYEDGGICIATNDITMEGDSFINKIH |
| | | FKGTNFPPNGPVMQKRTVGWEASTEKMYERDGVLKGDVKMKLLLKG |
| | | GGHYRCDYRTTYKVKQKPVKLPDYHFVDHRIEILSHDKDYNKVKLY |
| | | EHAVARNSTDSMDELYKGGSGGMVSKGEETITSVIKPDMKNKLRME |
| | | GNVNGHAFVIEGEGSGKPFEGIQTIDLEVKEGAPLPFAYDILTTAF |
| | | HYGNRVFTKYPR |
| 129 | TEV | GTENLYFQS |
| | (tobacco | |
| | Etch Virus) | |
| | cleavage site | |
| 130 | Linker | GSGGTENLYFQSGTSGGA |
| | comprising | |
| | a TEV | |

In some embodiments, an iAD, e.g., iADAR fusion protein comprises an AD-DDN associated with a first member of the binding pair (e.g., BP1), and an AD-DDC associated with a second member of the binding pair (e.g., BP2). In some embodiments, the BP1 is a SpyCatcher domain (e.g., SEQ ID NO: 127, or a polypeptide that that is at least 85% sequence identity to SEQ ID NO: 127. In some embodiments, where BP1 is a SpyCatcher domain, the BP2 is a Spy Tag which is associated with the AD-DDC, where the Spy Tag comprised amino acids of SEQ ID NO: 126 or a polypeptide that that is at least 85% sequence identity to SEQ ID NO: 126. In some embodiments, there is a cleavable linker located between the AD-DDN and the BP1. In alternative embodiments, there is a cleavable linker located between the AD-DDC and BP2. In some embodiments, there is a cleavable linker located between the BP1 and its attachment to the AD-DD or AD-DDN, and/or a cleavable linker located between BP2 and its attachment to AD-DD or AD-DDC.

In some embodiments the cleavable linkage comprises a Tobacco Etch Virus cleavage site (e.g., SEQ ID NO: 129). In some embodiments, the cleavable linker located between the AD-DDC and the BP2 (e.g., a SpyCatcher domain or other BP2 as disclosed herein) comprises SEQ ID NO: 130, or a linker that is at least 85% sequence identity to SEQ ID NO: 130. In some embodiments, the cleavable linker is cleaved by TEV protease (SEQ ID NO: 36)

In some embodiments, a cleavable linker located between the BP2 (e.g., Spy Catcher domain) and AD-DDC is a cleavable linker that is cleaved by light at a specific wavelength. In some embodiments, a cleavable linker that is cleaved by light at a specific wavelength and is located between the AD-DDC and the BP2 (e.g. a SpyCatcher domain) is PhoCl comprises SEQ ID NO: 130, or a linker that is at least 85% sequence identity to SEQ ID NO: 130.

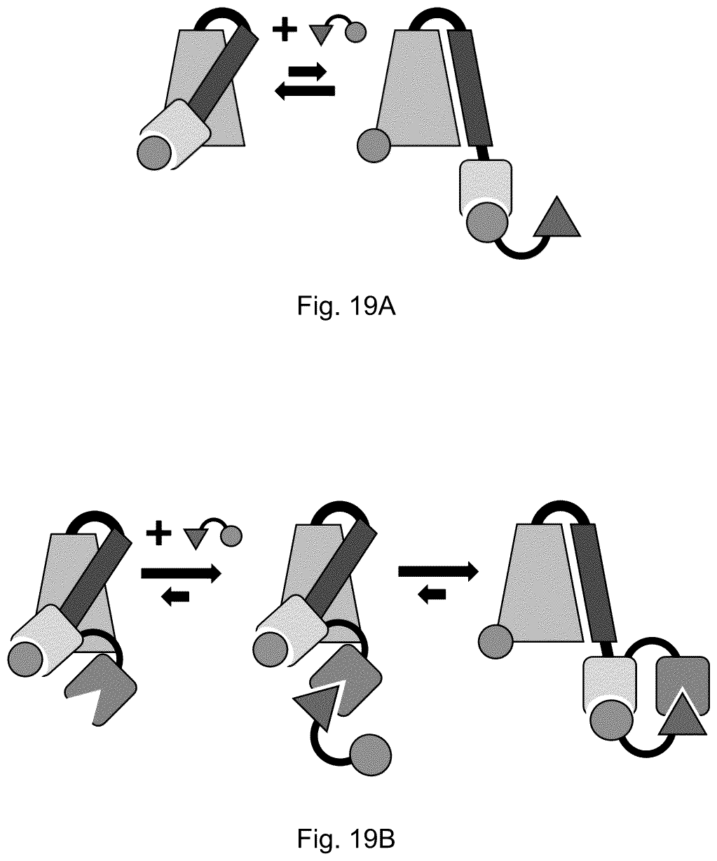

In some embodiments, the iADAR comprises a binding domain (BP1′), which is in addition to the first binding domain (BP1); the additional binding domain can localize the inducer to iADAR and increase sensitivity (see e.g., Example 10, FIG. 19A-19B). Such an additional protein binding domain can increase the local concentration of inducers and reduce the amount of inducer necessary to activate an iADAR.

As a non-limiting example, in antigen activating iADAR systems, fusing an additional antigen binding domain (BP1′) that binds a distinct epitope (BP2′) from the other antigen binding domain (BP1, which binds to the epitope BP2) serves to bind the antigen (BP2′) and increase the local concentration of the inducer, leading to increased sensitivity.

As a non-limiting example, in protease activating iADAR systems, an antigen binding domain (BP1′) would bring the protease (BP1) in close proximity to its substrate (BP2) and increase the efficiency and catalytic rate of the cleavage.

The additional binding domain (BP1′) can be any of the binding domains described herein, as long as the additional binding domain (BP1′) and its cognate antigen (BP2′) is different and distinct from the first affinity binding pair (e.g., BP1 and BP2). Non-limiting examples of the additional binding domain (BP1′) include: Bcl-XL (SEQ ID NO: 117), Mcl-1 (SEQ ID NO: 120), Bcl-XL (SEQ ID NO: 117), NbALFA (SEQ ID NO: 123), SpyCatcher (SEQ ID NO: 127).

The additional binding domain (BP1′) binds to its cognate binding domain (BP2′), non-limiting examples of which include Bim ((SEQ ID NO: 118), MS1(I) (SEQ ID NO: 119), Bad(L) (SEQ ID NO: 121), BAD (SEQ ID NO: 116), Bad(F) ((SEQ ID NO: 115), ALFA (SEQ ID NO: 122), ALFA-PE ((SEQ ID NO: 124), ALFA-78 ((SEQ ID NO: 125), SpyTag (SEQ ID NO: 126) (see e.g., Tables 4A-4B).

In some embodiments, the additional binding domain (BP1′) is a repressible protease as described further herein (e.g., NS3), and its cognate antigen (BP2′) is a peptide (e.g., an NS3-binding peptide such as ANR) as described further herein.

In some embodiments, the additional binding domain (BP1′) is linked to the first binding domain (BP1) directly or indirectly via a linker. In some embodiments, the cognate antigen (BP2′, which binds to the additional binding domain, BP1′) is linked to the inducer (for the BP1 and BP2 binding pair), directly or indirectly via a linker.

i. Inducers of the Affinity Binding Pair

Depending on the affinity binding pair of the iADAR, inducers can be, but are not limited to, small molecules, proteases, light-inducible control, sound inducible control, cell cycle dependent, ultrasound or other wavelength dependent, heat-activated triggers, antibodies, endogenous triggers, disease triggers, external triggers and cell-specific marker triggers, and the like.

Non-limiting examples of small molecule inducers include A-1331852, ABT-737, and S63845 as described further herein. In embodiments using a repressible protease and its cognate protease domain as the binding pair of the iADAR, the inducer can be a protease inhibitor, e.g., selected from grazoprevir, danoprevir, simeprevir, asunaprevir, ciluprevir, boceprevir, sovaprevir, paritaprevir, ombitasvir, paritaprevir, ritonavir, dasabuvir, and telaprevir or Table 9.

ii. Repressible Protease

In some embodiments, the affinity binding pair comprises a repressible protease, such as NS3, that binds to a peptide domain. See e.g., US20230159600A1 and US20220098246A1, which are incorporated herein by reference in their entireties.

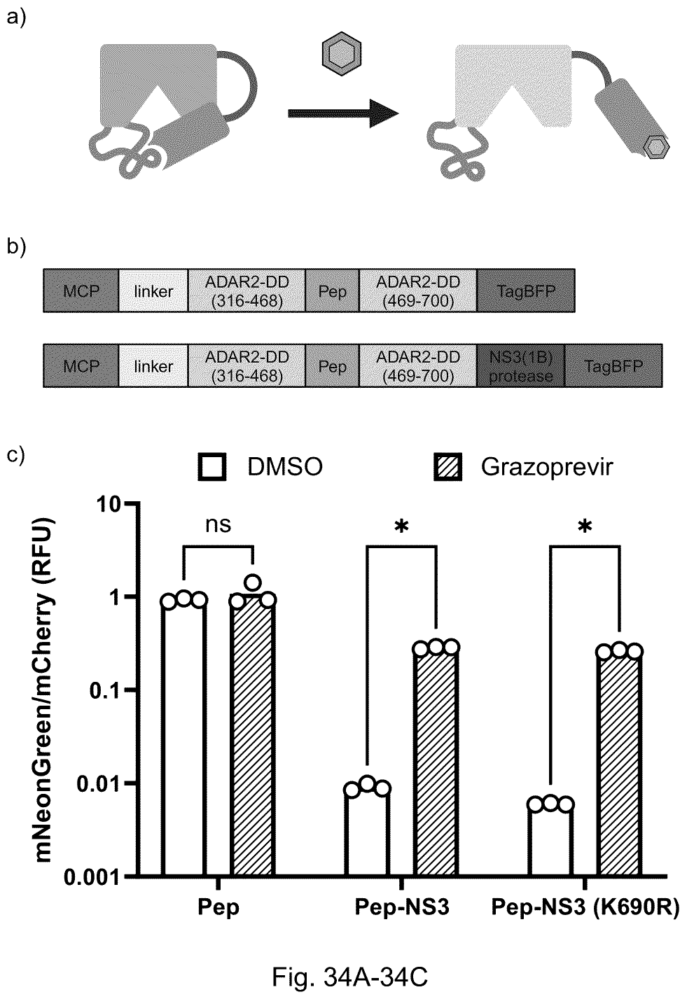

In one aspect described herein is a fusion protein comprising: (a) a first portion of a deaminase domain (DD) of an adenosine deaminase; (b) a repressible protease associated with the first portion of the DD; (c) a second portion of the DD; and (d) a protease-binding peptide associated with the second portion of the DD (see e.g., FIG. 34A-34C). In some embodiments, the repressible protease is capable of binding to the protease-binding peptide in the absence of an inhibitor for the repressible protease, resulting in allosteric inhibition of the first and second portions of the DD. In some embodiments, the repressible protease is not capable of binding to the protease-binding peptide in the presence of the inhibitor for the repressible protease, resulting in activation of the first and second portions of the DD.

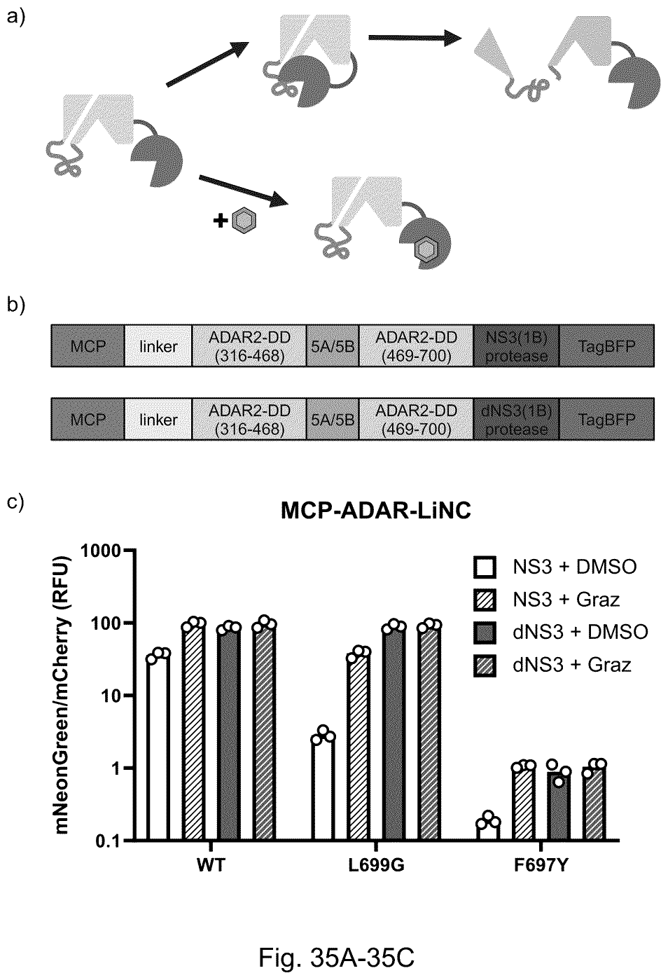

In one aspect described herein is a fusion protein comprising: (a) a first portion of a deaminase domain (DD) of an adenosine deaminase; (b) a repressible protease associated with the first portion of the DD; (c) a second portion of the DD; and (d) a protease cleavage site associated with the first and second portions of the DD (see e.g., FIG. 35A-35C). In some embodiments, the repressible protease is capable of binding to the protease cleavage site in the absence of an inhibitor for the repressible protease, resulting in cleavage of the protease cleavage site and inactivation of the first and second portions of the DD. In some embodiments, the repressible protease is not capable of binding to the protease cleavage site in the presence of the inhibitor for the repressible protease, resulting in activation of the first and second portions of the DD.

As used herein, the term “repressible protease” refers to a protease that can be inactivated by the presence or absence of a specific agent (e.g., that specifically binds to the protease). In some embodiments, a repressible protease is active (e.g., binds to a peptide domain) in the absence of the specific agent and is inactive (e.g., does not bind to a peptide domain) in the presence of the specific agent. In some embodiments, the specific agent is a protease inhibitor. In some embodiments, the protease inhibitor specifically inhibits a given repressible protease as described herein.

In some embodiments of any of the aspects, an iAD polypeptide as described herein (or an iADAR polypeptide system collectively) comprises 1, 2, 3, 4, 5, or more repressible protease(s). In some embodiments of any of the aspects, the iAD polypeptide or system comprises one repressible protease. In embodiments comprising multiple repressible proteases, the multiple repressible proteases can be different individual repressible proteases or multiple copies of the same repressible protease, or a combination of the foregoing.

Non-limiting examples of repressible proteases include hepatitis C virus proteases (e.g., NS3 and NS2-3); HIV protease; HIV1 protease; coronavirus (main) protease; SARS-CoV2 protease; Tobacco etch virus (TEV) protease; signal peptidase; proprotein convertases of the subtilisin/kexin family (furin, PCI, PC2, PC4, PACE4, PC5, PC); proprotein convertases cleaving at hydrophobic residues (e.g., Leu, Phe, Val, or Met); proprotein convertases cleaving at small amino acid residues such as Ala or Thr; proopiomelanocortin converting enzyme (PCE); chromaffin granule aspartic protease (CGAP); prohormone thiol protease; carboxypeptidases (e.g., carboxypeptidase E/H, carboxypeptidase D and carboxypeptidase Z); aminopeptidases (e.g., arginine aminopeptidase, lysine aminopeptidase, aminopeptidase B); prolyl endopeptidase; aminopeptidase N; insulin degrading enzyme; calpain; high molecular weight protease; and, caspases 1, 2, 3, 4, 5, 6, 7, 8, and 9. Other proteases include, but are not limited to, aminopeptidase N; puromycin sensitive aminopeptidase; angiotensin converting enzyme; pyroglutamyl peptidase II; dipeptidyl peptidase IV; N-arginine dibasic convertase; endopeptidase 24.15; endopeptidase 24.16; amyloid precursor protein secretases alpha, beta and gamma; angiotensin converting enzyme secretase; TGF alpha secretase; T F alpha secretase; FAS ligand secretase; TNF receptor-I and -II secretases; CD30 secretase; KL1 and KL2 secretases; IL6 receptor secretase; CD43, CD44 secretase; CD 16-1 and CD 16-11 secretases; L-selectin secretase; Folate receptor secretase; MMP 1, 2, 3, 7, 8, 9, 10, 11, 12, 13, 14, and 15; urokinase plasminogen activator; tissue plasminogen activator; plasmin; thrombin; BMP-1 (procollagen C-peptidase); ADAM 1, 2, 3, 4, 5, 6, 7, 8, 9, 10, and 11; and, granzymes A, B, C, D, E, F, G, and H. For a discussion of proteases, see, e.g., V. Y. H. Hook, Proteolytic and cellular mechanisms in prohormone and proprotein processing, RG Landes Company, Austin, Tex., USA (1998); N. M. Hooper et al., Biochem. J. 321: 265-279 (1997); Z. Werb, Cell 9 1: 439-442 (1997); T. G. Wolfsberg et al., J. Cell Biol. 131: 275-278 (1995); K. Murakami and J. D. Etlinger, Biochem. Biophys. Res. Comm. 146: 1249-1259 (1987); T. Berg et al., Biochem. J. 307: 313-326 (1995); M. J. Smyth and J. A. Trapani, Immunology Today 16: 202-206 (1995); R. V. Talanian et al., J. Biol. Chem. 272: 9677-9682 (1997); and N. A. Thomberry et a, J. Biol. Chem. 272: 17907-1791 1 (1997); International Patent Application WO2019118518; Rajakuberan et al., Methods Mol Biol. 2012; 903:393-405; Gao et al. Science 21 Sep. 2018: Vol. 361, Issue 6408, pp. 1252-1258; Tague et al., Nat Methods. 2018 July; 15(7):519-522; Lin et al. PNAS Jun. 3, 2008 105 (22) 7744-7749; U.S. patent application Ser. No. 16/832,751 filed Mar. 27, 2020; the contents of each of which are incorporated herein by reference in their entireties.

In some embodiments of any of the aspects, the repressible protease is hepatitis C virus (HCV) nonstructural protein 3 (NS3). NS3, also known as p-70, is a viral nonstructural protein that is a 70 kDa cleavage product of the hepatitis C virus polyprotein. The 631-residue HCV NS3 protein is a dual-function protein, containing the trypsin/chymotrypsin-like serine protease in the N-terminal region and a helicase and nucleoside triphosphatase in the C-terminal region. The minimal sequences required for a functional serine protease activity comprise the N-terminal 180 amino acids of the NS3 protein, which can also be referred to as “NS3a”. Deletion of up to 14 residues from the N terminus of the NS3 protein is tolerated while maintaining the serine protease activity. Accordingly, the repressible proteases described herein comprise at the least residues 14-180 of the wildtype NS3 protein.

HCV has at least seven genotypes, labeled 1 through 7, which can also be further designated with “a” and “b” subtypes. Accordingly, the repressible protease can be an HCV genotype 1 NS3, an HCV genotype 1a NS3, an HCV genotype 1b NS3, an HCV genotype 2 NS3, an HCV genotype 2a NS3, an HCV genotype 2b NS3, an HCV genotype 3 NS3, an HCV genotype 3a NS3, an HCV genotype 3b NS3, an HCV genotype 4 NS3, an HCV genotype 4a NS3, an HCV genotype 4b NS3, an HCV genotype 5 NS3, an HCV genotype 5a NS3, an HCV genotype 5b NS3, an HCV genotype 6 NS3, an HCV genotype 6a NS3, an HCV genotype 6b NS3, an HCV genotype 7 NS3, an HCV genotype 7a NS3, or an HCV genotype 7b NS3. In some embodiments of any of the aspects, the repressible protease can be any known HCV NS3 genotype, variant, or mutant, e.g., that maintains the same function. In some embodiments of any of the aspects, the NS3 sequence comprises residues 1-180 of the NS3 protein from HCV-H, HCV-1, HCV-J1, HCV-BK, HCV-JK1, HCV-J4, HCV-J, HCV-J6, C14112, HCV-J8, D14114, HCV-Nz11, or HCV-K3a (see e.g., Chao Lin, Chapter 6: HCV NS3-4A Serine Protease, Hepatitis C Viruses: Genomes and Molecular Biology, Editor: Tan SL, Norfolk (UK): Horizon Bioscience, 2006; the content of which is incorporated herein by reference in its entirety). In some embodiments of any of the aspects, the repressible protease is a chimera of 2, 3, 4, 5, or more different NS3 genotypes, variants, or mutants as described herein, such that the protease maintains its cleavage and/or binding functions.

In some embodiments of any of the aspects, the repressible protease of an iAD polypeptide as described herein comprises SEQ ID NOs: 208-224 or an amino acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 208-224 that maintains the same function.

In some embodiments of any of the aspects, the repressible protease of an iAD polypeptide as described herein does not comprise at most the first (i.e., N-terminal) residues of SEQ ID NOs: 208-224. In some embodiments of any of the aspects, the repressible protease of an iAD polypeptide as described herein comprises residues 1-180, 2-180, 3-180, 4-180, 5-180, 6-180, 7-180, 8-180, 9-180, 10-180, 11-180, 12-180, 13-180, 14-180, 15-180, 16-180, 17-180, 18-180, 19-180, 20-180, 21-180, 22-180, 23-180, 24-180, 25-180, 26-180, 27-180, 28-180, 29-180, or 30-180 of SEQ ID NOs: 208-224.

NS3 (genotype 1A), 189 aa; bold text indicates
His-57 of the catalytic triad; italicized
double underlined text indicates Asp-81 of
the catalytic triad; bold italicized
indicates Ser-139 of the catalytic triad;
double underlined text indicates Asp-168.
SEQ ID NO: 208
APITAYAQQTRGLLGCIITSLTGRDKNQVEGEVQIVSTAT
QTFLATCINGVCWAVYHGAGTRTIASPKGPVIQMYTNVD
Q [symbol] LVGWPAPQGSRSLTPCTCGSSDLYLVTRHADVIPVRR
RGDSRGSLLSPRPISYLKGSSGGPLLCPAGHAVGLFRAA
VCTRGVAKAVDFIPVENLETTMRSPVFTDNSS,
NS3 protease domain (genotype 1A)
SEQ ID NO: 209
APITAYAQQTRGLLGCIITSLTGRDKNQVEGEVQIVSTATQTFLA
TCINGVCWAVYHGAGTRTIASPKGPVIQMYTNVDQDLVGWPAPQG
SRSLTPCTCGSSDLYLVTRHADVIPVRRRGDSRGSLLSPRPISYL
KGSSGGPLLCPAGHAVGLFRAAVCTRGVAKAVDFIPVENLETTMR
SPVFTD,
NS3 (genotype 1A), 180 aa (see e.g.,
residues 1027-1206 of Hepatitis C
virus genotype 1 polyprotein, NCBI
Reference Sequence: NP_671491.1.
SEQ ID NO: 210
APITAYAQQTRGLLGCIITSLTGRDKNQVEGEVQIVSTATQTFLA
TCINGVCWTVYHGAGTRTIASPKGPVIQMYTNVDQDLVGWPAPQG
SRSLTPCTCGSSDLYLVTRHADVIPVRRRGDSRGSLLSPRPISYL
KGSSGGPLLCPAGHAVGLFRAAVCTRGVAKAVDFIPVENLETTMR,
NS3 (genotype 1B), 180 aa
(see e.g., residues 1-180 Chain A, Ns3
Protease, PDB: 4K8B_A)
SEQ ID NO: 211
APITAYSQQTRGLLGCIITSLTGRDKNQVEGEVQVVSTATQSFLA
TCVNGVCWTVYHGAGSKTLAGPKGPITQMYTNVDQDLVGWQAPPG
ARSLTPCTCGSSDLYLVTRHADVIPVRRRGDSRGSLLSPRPVSYL
KGSSGGPLLCPSGHAVGIFRAAVCTRGVAKAVDFVPVESMETTMR,
NS3 (genotype 2), 180 aa
(see e.g., residues 1031-1210 of Hepatitis C
virus genotype 2 polyprotein, NCBI Reference
Sequence: YP_001469630.1
SEQ ID NO: 212
APITAYAQQTRGLLGTIVVSMTGRDKTEQAGEIQVLSTVTQSFLG
TSISGVLWTVYHGAGNKTLAGSRGPVTQMYSSAEGDLVGWPSPPG
TKSLEPCTCGAVDLYLVTRNADVIPARRRGDKRGALLSPRPLSTL
KGSSGGPVLCPRGHAVGVFRAAVCSRGVAKSIDFIPVETLDIVTR,
NS3 (genotype 3), 180 aa
(see e.g., residues 1033-1212 of Hepatitis C
virus genotype 3 polyprotein, NCBI Reference
Sequence: YP_001469631.1)
SEQ ID NO: 213
APITAYAQQTRGLLGTIVTSLTGRDKNVVTGEVQVLSTATQTFLG
TTVGGVIWTVYHGAGSRTLAGAKHPALQMYTNVDQDLVGWPAPPG
AKSLEPCACGSSDLYLVTRDADVIPARRRGDSTASLLSPRPLACL
KGSSGGPVMCPSGHVAGIFRAAVCTRGVAKSLQFIPVETLSTQAR,
NS3 (genotype 4), 180 aa (see e.g.,
residues 1027-1206 of Hepatitis C
virus genotype 4 polyprotein, NCBI
Reference Sequence: YP_001469632.1)
SEQ ID NO: 214
APITAYAQQTRGLFSTIVTSLTGRDTNENCGEVQVLSTATQSFLG
TAVNGVMWTVYHGAGAKTISGPKGPVNQMYTNVDQDLVGWPAPPG
VRSLAPCTCGSADLYLVTRHADVIPVRRRGDTRGALLSPRPISIL
KGSSGGPLLCPMGHRAGIFRAAVCTRGVAKAVDFVPVESLETTMR,
NS3 (genotype 5), 180 aa (see e.g.,
residues 1028-1207 of Hepatitis C
virus genotype 5 polyprotein, NCBI
Reference Sequence: YP_001469633.1)
SEQ ID NO: 215
APITAYAQQTRGVLGAIVLSLTGRDKNEAEGEVQFLSTATQTFLG
ICINGVMWTLFHGAGSKTLAGPKGPVVQMYTNVDKDLVGWPSPPG
KGSLTRCTCGSADLYLVTRHADVIPARRRGDTRASLLSPRPISYL
KGSSGGPIMCPSGHVVGVFRAAVCTRGVAKALEFVPVENLETTMR,
NS3 (genotype 6), 180 aa (see e.g.,
residues 1032-1211 of Hepatitis C
virus genotype 6 polyprotein, NCBI
Reference Sequence: YP_001469634.1)
SEQ ID NO: 216
APITAYAQQTRGLVGTIVTSLTGRDKNEAEGEVQVVSTATQSFLA
TTINGVLWTVYHGAGSKNLAGPKGPVCQMYTNVDQDLVGWPAPLG
ARSLAPCTCGSSDLYLVTRGADVIPARRRGDTRAALLSPRPISTL
KGSSGGPLMCPSGHVVGLFRAAVCTRGVAKALDFIPVENMDTTMR,
NS3 (genotype 7), 180 aa (see e.g.,
residues 1031-1210 of Hepatitis C
virus genotype 7 polyprotein, NCBI
Reference Sequence: YP_009272536.1)
SEQ ID NO: 217
APISAYAQQTRGLISTLVVSLTGRDKNETAGEVQVLSTSTQTFLG
TNVGGVMWGPYHGAGTRTVAGRGGPVLQMYTSVSDDLVGWPAPPG
SKSLEPCSCGSADLYLVTRNADVLPLRRKGDGTASLLSPRPVSSL
KGSSGGPVLCPQSHCVGIFRAAVCTRGVAKAVQFVPIEKMQVAQR,
NS3 genotype la (HCV-H), 180 aa
SEQ ID NO: 218
APITAYAQQTRGLLGCIITSLTGRDKNQVEGEVQIVSTATQTFLA
TCINGVCWTVYHGAGTRTIASPKGPVIQMYTNVDQDLVGWPAPQG
SRSLTPCTCGSSDLYLVTRHADVIPVRRRGDSRGSLLSPRPISYL
KGSSGGPLLCPAGHAVGLFRAAVCTRGVTKAVDFIPVENLETTMR,
NS3 genotype 1b (HCV-BK), 180 aa
SEQ ID NO: 219
APITAYSQQTRGLLGCIITSLTGRDKNQVEGEVQVVSTATQSFLA
TCVNGVCWTVYHGAGSKTLAAPKGPITQMYTNVDQDLVGWPKPPG
ARSLTPCTCGSSDLYLVTRHADVIPVRRRGDSRGSLLSPRPVSYL
KGSSGGPLLCPFGHAVGIFRAAVCTRGVAKAVDFVPVESMETTMR,
NS3 genotype 2a (HCV-J6), 180 aa
SEQ ID NO: 220
APITAYAQQTRGLLGTIVVSMTGRDKTEQAGEIQVLSTVTQSFLG
TTISGVLWTVYHGAGNKTLAGSRGPVTQMYSSAEGDLVGWPSPPG
TKSLEPCTCGAVDLYLVTRNADVIPARRRGDKRGALLSPRPLSTL
KGSSGGPVLCPRGHAVGVFRAAVCSRGVAKSIDFIPVETLDIVTR,
NS3 genotype 2b (HCV-J8), 180 aa
SEQ ID NO: 221
APITAYTQQTRGLLGAIVVSLTGRDKNEQAGQVQVLSSVTQTFLG
TSISGVLWTVYHGAGNKTLAGPKGPVTQMYTSAEGDLVGWPSPPG
TKSLDPCTCGAVDLYLVTRNADVIPVRRKDDRRGALLSPRPLSTL
KGSSGGPVLCSRGHAVGLFRAAVCARGVAKSIDFIPVESLDVATR,
NS3 genotype 3a (HCV-Nz11), 180 aa
SEQ ID NO: 222
APITAYAQQTRGLLGTIVTSLTGRDKNVVTGEVQVLSTATQTFLG
TTVGGVIWTVYHGAGSRTLAGAKHPALQMYTNVDQDLVGWPAPPG
AKSLEPCACGSSDLYLVTRDADVIPARRRGDSTASLLSPRPLACL
KGSSGGPVMCPSGHVAGIFRAAVCTRGVAKSLQFIPVETLSTQAR,

In some embodiments of any of the aspects, a repressible protease as described herein is resistant to 1, 2, 3, 4, 5, or more different protease inhibitors as described herein. Non-limiting examples of NS3 amino acid substitutions conferring resistance to HCV NS3 protease inhibitors include: V36L (e.g., genotype 1b), V36M (e.g., genotype 2a), T54S (e.g., genotype 1b), Y56F (e.g., genotype 1b), Q80L (e.g., genotype 1b), Q80R (e.g., genotype 1b), Q80K (e.g., genotype 1a, 1b, 6a), Y1321 (e.g., genotype 1b), A156S (e.g., genotype 2a), A156G, A156T, A156V, D168A (e.g., genotype 1b), 1170V (e.g., genotype 1b), S20N, R26K, Q28R, A39T, Q41R, I71V, Q80R, Q86R, P89L, P89S, S101N, A111S, P115S, S122R, R155Q, L144F, A150V, R155W, V158L, D168A, D168G, D168H, D168N, D168V, D168E, D168Y, E176K, T178S, M179I, M179V, and M179T. See e.g., Sun et al., Gene Expr. 2018, 18(1): 63-69; Kliemann et al., World J Gastroenterol. 2016 Oct. 28, 22(40): 8910-8917; U.S. Pat. Nos. 7,208,309; 7,494,660; the contents of each of which are incorporated herein by reference in their entireties.

In some embodiments of any of the aspects, an iAD polypeptide as described herein comprises an NS3 protease comprising at least one resistance mutation as described herein or any combination thereof. In some embodiments of any of the aspects, an iAD polypeptide as described herein comprises an NS3 protease that is resistant to one protease inhibitor but responsive to at least one other protease inhibitor. In some embodiments of any of the aspects, an iAD system comprises: (a) a first iAD polypeptide comprising a repressible protease (e.g., NS3) that is resistant to a first protease inhibitor and that is susceptible to a second protease inhibitor; and (b) a second iAD polypeptide comprising a repressible protease (e.g., NS3) that is susceptible to a first protease inhibitor and that is resistant to a second protease inhibitor. Accordingly, presence of the first protease inhibitor can modulate the activity of the second iAD polypeptide but not the first iAD polypeptide, while the presence of the second protease inhibitor can modulate the activity of the first iAD polypeptide but not the second iAD polypeptide.

In some embodiments of any of the aspects, a repressible protease as described herein is sensitive to 1, 2, 3, 4, 5, or more different protease inhibitors as described herein. In some embodiments of any of the aspects, the NS3 protease comprises at least one of the following mutations: V36M, T54A, S122G, F43L, Q80K, S122R, D168Y, or any combination thereof. In some embodiments of any of the aspects, the NS3 protease comprises at least one of the following mutations: V36M, T54A, S122G, or any combination thereof; such a protease is also referred to herein as NS3^{AI}, as these mutations increase its sensitivity to asunaprevir (see e.g., SEQ ID NO: 223). In some embodiments of any of the aspects, the NS3 protease comprises at least one of the following mutations: F43L, Q80K, S122R, D168Y, or any combination thereof; such a protease is also referred to herein as NS3^{TI}, as these mutations increase its sensitivity to telaprevir (see e.g., SEQ ID NO: 224). See e.g., WO2019023164; Jacobs et al., StaPLs: versatile genetically encoded modules for engineering drug-inducible proteins, Nat Methods. 2018 July; 15(7): 523-526; the contents of each of each are incorporated herein by reference in their entireties.

NS3AI; the V36M, T54A, S122G mutations
are shown in bold double
underlined text, respectively
SEQ ID NO: 223
APITAYAQQTRGLLGCIITSLTGRDKNQVEGEVQIMSTATQTFLA
TCINGVCWAVYHGAGTRTIASPKGPVIQMYTNVDQDLVGWPAPQG
SRSLTPCTCGSSDLYLVTRHADVIPVRRRGDGRGSLLSPRPISYL
KGSSGGPLLCPAGHAVGLFRAAVCTRGVAKAVDFIPVENLETTMR
SPVFTD,
NS3TI; the F43L, Q80K, S122R, D168Y
mutations are shown in bold double
underlined text, respectively
SEQ ID NO: 224
APITAYAQQTRGLLGCIITSLTGRDKNQVEGEVQIVSTATQTLLA
TCINGVCWAVYHGAGTRTIASPKGPVIQMYTNVDKDLVGWPAPQG
SRSLTPCTCGSSDLYLVTRHADVIPVRRRGDRRGSLLSPRPISYL
KGSSGGPLLCPAGHAVGLFRAAVCTRGVAKAVYFIPVENLETTMR
SPVFTD,

In some embodiments of any of the aspects, the polypeptide further comprising a cofactor for the repressible protease. As used herein the term “cofactor for the repressible protease” refers to a molecule that increases the activity of the repressible protease. In some embodiments of any of the aspects, an iAD polypeptide as described herein comprises 1, 2, 3, 4, 5, or more cofactors for the repressible protease. In some embodiments of any of the aspects, the iAD polypeptide comprises one cofactor for each repressible protease. In embodiments comprising multiple cofactors for the repressible protease, the multiple cofactors for the repressible protease can be different individual cofactors or multiple copies of the same cofactor, or a combination of the foregoing.

In some embodiments of any of the aspects, the cofactor is an HSV NS4A domain, and the repressible protease is HSV NS3. The nonstructural protein 4a (NS4A) is the smallest of the nonstructural HCV proteins. The NS4A protein has multiple functions in the HCV life cycle, including (1) anchoring the NS3-4A complex to the outer leaflet of the endoplasmic reticulum and mitochondrial outer membrane, (2) serving as a cofactor for the NS3A serine protease, (3) augmenting NS3A helicase activity, and (4) regulating NS5A hyperphosphorylation and viral replication. The interactions between NS4A and NS4B control genome replication and between NS3 and NS4A play a role in virus assembly.

In some embodiments of any of the aspects, an iAD polypeptide as described herein comprises the portion of the NS4a polypeptide that serves as a cofactor for NS3. Deletion analysis has shown that the central region (approximately residues 21 to 34) of the 54-residue NS4A protein is essential and sufficient for the cofactor function of the NS3 serine protease. Accordingly, in some embodiments of any of the aspects, the repressible protease cofactor comprises a 14-residue region of the wildtype NS4A protein.

In some embodiments of any of the aspects, the cofactor for the repressible protease can be an HCV genotype 1 NS4A, an HCV genotype 1a NS4A, an HCV genotype 1b NS4A, an HCV genotype 2 NS4A, an HCV genotype 2a NS4A, an HCV genotype 2b NS4A, an HCV genotype 3 NS4A, an HCV genotype 3a NS4A, an HCV genotype 3b NS4A, an HCV genotype 4 NS4A, an HCV genotype 4a NS4A, an HCV genotype 4b NS4A, an HCV genotype 5 NS4A, an HCV genotype 5a NS4A, an HCV genotype 5b NS4A, an HCV genotype 6 NS4A, an HCV genotype 6a NS4A, an HCV genotype 6b NS4A, an HCV genotype 7 NS4A, an HCV genotype 7a NS4A, or an HCV genotype 7b NS4A. In some embodiments of any of the aspects, the cofactor for the repressible protease can be any known NS4A genotype, variant, or mutant, e.g., that maintains the same function. In some embodiments of any of the aspects, the NS4A sequence comprises residues 21-31 of the NS4A protein from HCV-H, HCV-1, HCV-J1, HCV-BK, HCV-JK1, HCV-J4, HCV-J, HCV-J6, C14112, HCV-J8, D14114, HCV-Nz11, or HCV-K3a (see e.g., Chao Lin 2006 supra; see e.g., Table 8).

In some embodiments of any of the aspects, the cofactor for a repressible protease of an iAD polypeptide as described herein comprises SEQ ID NOs: 48, 98, 137-156, or an amino acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 48, 98, 137-156 that maintains the same functions as one of SEQ ID NOs: 48, 98, 137-156. In some embodiments of any of the aspects, the cofactor for a repressible protease of an iAD polypeptide as described herein comprises SEQ ID NOs: 81, 93, 96, 255-276, or an amino acid sequence that is at least 95% identical to the sequence of one of SEQ ID NOs: 81, 93, 96, 255-276 that maintains the same function.

In some embodiments of any of the aspects, the cofactor for the repressible protease of an iAD polypeptide as described herein comprises residues 1-14, 1-13, 1-12, 1-11, 1-10, 2-14, 2-13, 2-12, 2-11, 2-10, 3-14, 3-13, 3-12, 3-11, 3-10, 4-14, 4-13, 4-12, 4-11, or 4-10 of any of SEQ ID NOs: 225-249.

SEQ ID NO: 225, NS4A (genotype 1A), 13 aa,
GCVVIVGRIVLSG
SEQ ID NO: 226, NS4A domain (genotype 1a)
STWVLVGGVLAALAAYCLSTGCVVIVGRIVLSGKPAIIPDREVLY
SEQ ID NO: 227, NS4
(with L6 linker in bold text)
STWVLVGGVLAALAAYCLSTGCVVIVGRIVLSGKP
AGSSGSSIIPDREVLY
SEQ ID NO: 228, NS4A domain,
IDTKYIMTCMSADLEVVTSTWVLVGGVLAALAAY
CLSTGCVVIVGRIVLSGKPAIIPDREVLY
SEQ ID NO: 229, NS4A (genotype 1B), 12 aa,
GSVVIVGRIILS;
see e.g., Chain C,
Nonstructural Protein, PDB: 4K8B C.
SEQ ID NO: 230, NS4A (genotype 1), 14 aa
(see e.g., residues 1678-1691 of Hepatitis C
virus genotype 1 polyprotein, NCBI Reference
Sequence: NP_671491.1):
GCVVIVGRIVLSGK
SEQ ID NO: 231, NS4A (genotype 2), 14 aa
(see e.g., residues 1682-1695 of Hepatitis C
virus genotype 2 polyprotein, NCBI Reference
Sequence: YP_001469630.1:
GCVCIIGRLHINQR
SEQ ID NO: 232, NS4A (genotype 3), 14 aa
(see e.g., residues 1684-1697 of Hepatitis C
virus genotype 3 polyprotein, NCBI Reference
Sequence: YP_001469631.1):
GCVVIVGHIELEGK
SEQ ID NO: 233, NS4A (genotype 4), 14 aa
(see e.g., residues 1678-1691 of Hepatitis C
virus genotype 4 polyprotein, NCBI Reference
Sequence: YP_001469632.1):
GSVVIVGRVVLSGQ
SEQ ID NO: 234, NS4A (genotype 5), 14 aa
(see e.g., residues 1679-1692 of Hepatitis C
virus genotype 5 polyprotein, NCBI Reference
Sequence: YP_001469633.1):
GSVAIVGRIILSGR
SEQ ID NO: 235, NS4A (genotype 6), 14 aa
(see e.g., residues 1683-1696 of Hepatitis C
virus genotype 6 polyprotein, NCBI Reference
Sequence: YP_001469634.1):
GCVVICGRIVTSGK
SEQ ID NO: 236, NS4A (genotype 7),
14 aa (see e.g., residues 1682-1695
of Hepatitis C
virus genotype 7 polyprotein, NCBI
Reference Sequence: YP_009272536.1):
GSVVVVGRVVLGSN

In some embodiments of any of the aspects, the NS4A sequence is selected from Table 5. In one embodiment, the NS4A comprises residues 21-31 of SEQ ID NO: 237-249 or a sequence that is at least 70% identical.

TABLE 8
Exemplary NS4A sequences
(see e.g., Chao Lin 2006 supra).
Residues 21-31 are bolded.

| SEQ | | |
|---|---|---|
| ID | Genotype | |
| NO | (strain) | Sequence |
| 237 | 1a (HCV-H) | STWVL VGGVL AALAA YCLST |
| | | GCVVI VGRIV LSGKP AIIPD |
| | | REVLY QEFDE MEEC |
| 238 | 1a (HCV-1) | STWVL VGGVL AALAA YCLST |
| | | GCVVI VGRVV LSGKP AIIPD |
| | | REVLY REFDE MEEC |
| 239 | 1a (HCV-J1) | STWVL VGGVL AALAA YCLST |
| | | GCVVI VGRIV LSGRP AIIPD |
| | | REVLY REFDE MEEC |
| 240 | 1b (HCV-BK) | STWVL VGGVL AALAA YCLTT |
| | | GSVVI VGRII LSGRP AIVPD |
| | | RELLY QEFDE MEEC |
| 241 | 1b (HCV-JK1) | STWVL VGGVL AALAA YCLTT |
| | | GSVVI VGRII LSGRP AIIPD |
| | | RELLY QEFDE MEEC |
| 242 | 1b (HCV-J4) | STWVL VGGVL AALAA YCLTT |
| | | GSVVI VGRII LSGKP AVVPD |
| | | RELLY QEFDE MEEC |
| 243 | 1b (HCV-J) | STWVL VGGVL AALAA YCLTT |
| | | GSVVI VGRII LSGRP AVIPD |
| | | RELLY REFDE MEEC |
| 244 | 2a (HCV-J6) | STWVL AGGVL AAVAA YCLAT |
| | | GCVCI IGRLH VNQRA VVAPD |
| | | KEVLY EAFDE MEEC |
| 245 | 2a (D14112) | STWVL AGGVL AAVAA YCLAT |
| | | GCVSI IGRLH INGRA VVAPD |
| | | KEVLY EAFDE MEEC |
| 246 | 2b (HCV-J8) | SSWVL AGGVL AAVAA YCLAT |
| | | GCISI IGRLH LNDRV VVAPD |
| | | KEILY EAFDE MEEC |
| 247 | 2b (D14114) | STWVL AGGVL AAVAA YCLAT |
| | | GCVSI IGRLH LNDQV VVTPD |
| | | KEILY EAFDE MEEC |
| 248 | 3a (HCV-Nz11) | STWVL LGGVL AALAA YCLSV |
| | | GCVVI VGHIE LEGKP ALVPD |
| | | KEVLY QQYDE MEEC |
| 249 | 3a (HCV-K3a) | STWVL LGGVL AAVAA YCLSV |
| | | GCVVI VGHIE LGGKP ALVPD |
| | | KEVLY QQYDE MEEC |

In some embodiments of any of the aspects, an iAD polypeptide as described herein can comprise any combination of NS3 and NS4A genotypes, variants, or mutants as described herein. In one embodiment, the NS3 and NS4A are selected from selected from the same genotype as each other. In some embodiments of any of the aspects, the NS3 is genotype 1a and the NS4A is genotype 1b. In some embodiments of any of the aspects, the NS3 is genotype 1b and the NS4A is genotype 1a.

In some embodiments of any of the aspects, an iAD polypeptide as described herein comprises an HSV NS4A domain adjacent to the NS3 repressible protease. In some embodiments of any of the aspects, the NS4A domain is N-terminal of the NS3 repressible protease. In some embodiments of any of the aspects, the NS4A domain is C-terminal of the NS3 repressible protease. In some embodiments of any of the aspects, the iAD polypeptide comprises a peptide linker between the NS4A domain and the NS3 repressible protease. Non-limiting examples of linker (e.g., between the NS4A domain and the NS3 repressible protease) include: SGTS (SEQ ID NO: 250) and GSGS (SEQ ID NO: 251).

In some embodiments of any of the aspects, any two domains as described herein in an iAD polypeptide can be joined into a single polypeptide by positioning a peptide linker, e.g., a flexible linker between them. As used herein “peptide linker” refers to an oligo- or polypeptide region from about 2 to 100 amino acids in length, which links together any of the sequences of the polypeptides as described herein. In some embodiments, linkers can include or be composed of flexible residues such as glycine and serine so that the adjacent protein domains are free to move relative to one another. Longer linkers may be used when it is desirable to ensure that two adjacent domains do not sterically interfere with one another. Linkers may be cleavable or non-cleavable.

In some embodiments of any of the aspects, the iAD comprises a TimeSTAMP domain (a time-specific tag for the age measurement of proteins). In some embodiments of any of the aspects, the TimeSTAMP comprises a repressible protease, at least one protease cleavage site, and a detectable marker. The detectable marker is removed from the iAD immediately after translation by the activity of the repressible protease until the time a protease inhibitor is added, after which newly synthesized iAD polypeptides retain their markers. TimeSTAMP allows for time-specific tagging of the age measurement of proteins, and allows sensitive and nonperturbative visualization and quantification of newly synthesized proteins of interest with exceptionally tight temporal control.

In some embodiments of any of the aspects, the repressible protease exhibits increased solubility compared to the wild-type protease. As a non-limiting example, the NS3 protease can comprise at least one of the following mutations or any combination thereof: Leu13 is substituted to Glu; Leu14 is substituted to Glu; Ile17 is substituted to Gln; Ile18 is substituted to Glu; and/or Leu21 is substituted to Gln. In some embodiments of any of the aspects, an iAD polypeptide as described herein comprises a repressible protease comprising SEQ ID NOs: 252-260, or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 307-315 that maintains the same functions (e.g., serine protease; increased solubility) as SEQ ID NOs: −252-260; see e.g., U.S. Pat. No. 6,333,186 and US Patent Publication US20020106642, the contents of each are incorporated herein by reference in their entireties.

soluble NS3, 182 aa
SEQ ID NO: 252
MAPITAYAQQTRGLLGCIITSLTGRDKNQVEGEVQIVSTAAQTFL
ATCINGVCWTVYHGAGTRTIASPKGPVIQMYTNVDKDLVGWPAPQ
GSRSLTPCTCGSSDLYLVTRHADVIPVRRRGDSRGSLLSPRPISY
LKGSSGGPLLCPAGHAVGIFRAAVCTRGVAKAVDFIPVESLETTM
RS,
soluble NS3/NS4A, 195 aa
SEQ ID NO: 253
MKKKGSVVIVGRIVLNGAYAQQTRGLLGCIITSLTGRDKNQVEGE
VQIVSTAAQTFLATCINGVCWTVYHGAGTRTIASPKGPVIQMYTN
VDKDLVGWPAPQGSRSLTPCTCGSSDLYLVTRHADVIPVRRRGDS
RGSLLSPRPISYLKGSSGGPLLCPAGHAVGIFRAAVCTRGVAKAV
DFIPVESLETTMRSP,
soluble NS3/NS4A, 195 aa
SEQ ID NO: 254
MKKKGSVVIVGRIVLNGAYAQQTRGEEGCQETSQTGRDKNQVEGE
VQIVSTAAQTFLATCINGVCWTVYHGAGTRTIASPKGPVIQMYTN
VDKDLVGWPAPQGSRSLTPCTCGSSDLYLVTRHADVIPVRRRGDS
RGSLLSPRPISYLKGSSGGPLLCPAGHAVGIFRAAVCTRGVAKAV
DFIPVESLETTMRSP,
soluble NS3/NS4A, 197 aa
SEQ ID NO: 255
MKKKGSVVIVGRINLSGDTAYAQQTRGEEGCQETSQTGRDKNQVE
GEVQIVSTAAQTFLATCINGVCWTVYHGAGTRTIASPKGPVIQMY
TNVDKDLVGWPAPQGSRSLTPCTCGSSDLYLVTRHADVIPVRRRG
DSRGSLLSPRPISYLKGSSGGPLLCPAGHAVGIFRAAVCTRGVAK
AVDFIPVESLETTMRSP,
soluble NS3/NS4A, 197 aa
SEQ ID NO: 256
MKKKGSVVIVGRINLSGDTAYAQQTRGEEGCQETSQTGRDKNQVE
GEVQIVSTATQTFLATCINGVCWTVYHGAGTRTIASPKGPVTQMY
TNVDKDLVGWQAPQGSRSLTPCTCGSSDLYLVTRHADVIPVRRRG
DSRGSLLSPRPISYLKGSSGGPLLCPAGHAVGIFRAAVCTRGVAK
AVDFIPVESLETTMRSP
soluble NS3/NS4A, 197 aa
SEQ ID NO: 257
MKKKGSVVIVGRINLSGDTAYAQQTRGEEGCQETSQTGRDKNQVE
GEVQIVSTATQTFLATSINGVLWTVYHGAGTRTIASPKGPVTQMY
TNVDKDLVGWQAPQGSRSLTPCTCGSSDLYLVTRHADVIPVRRRG
DSRGSLLSPRPISYLKGSSGGPLLCPAGHAVGIFRAAVSTRGVAK
AVDFIPVESLETTMRSP
soluble NS3/NS4A, 197 aa
SEQ ID NO: 258
MKKKGSVVIVGRINLSGDTAYAQQTRGEQGCQKTSHTGRDKNQVE
GEVQIVSTATQTFLATSINGVLWTVYHGAGTRTIASPKGPVTQMY
TNVDKDLVGWQAPQGSRSLTPCTCGSSDLYLVTRHADVIPVRRRG
DSRGSLLSPRPISYLKGSSGGPLLCPAGHAVGIFRAAVSTRGVAK
AVDFIPVESLETTMRSP
soluble NS3/NS4A, 197 aa
SEQ ID NO: 259
MKKKGSVVIVGRINLSGDTAYAQQTRGEQGTQKTSHTGRDKNQVE
GEVQIVSTATQTFLATSINGVLWTVYHGAGTRTIASPKGPVTQMY
TNVDKDLVGWQAPQGSRSLTPCTCGSSDLYLVTRHADVIPVRRRG
DSRGSLLSPRPISYLKGSSGGPLLCPAGHAVGIFRAAVSTRGVAK
AVDFIPVESLETTMRSP
NS3aH1, soluble NS3/NS4A (S139A), 196 aa
SEQ ID NO: 260
KKKGSVVIVGRINLSGDTAYAQQTRGEEGCQETSQTGRDKNQVEG
EVQIVSTATQTFLATSINGVLWTVYHGAGTRTIASPKGPVTQMYT
NVDKDLVGWQAPQGSRSLTPCTCGSSDLYLVTRHADVIPVRRRGD
SRGSLLSPRPISYLKGSAGGPLLCPAGHAVGIFRAAVSTRGVAKA
VDFIPVESLETTMRSP,

In some embodiments of any of the aspects, the repressible protease comprises mutations to increase binding affinity for a specific ligand. As a non-limiting example, NS3aH1 (e.g., SEQ ID NO: 260) comprises four mutations needed for interaction with the ANR peptide (e.g., SEQ ID NO: 261, GELDELVYLLDGPGYDPIHSD): A7S, E13L, I35V and T42S. Accordingly, in some embodiments of any of the aspects, a repressible protease as described herein comprises at least one of the following mutations: A7S, E13L, I35V and T42S, or any combination thereof.

In some embodiments of any of the aspects, an iAD polypeptide as described herein comprises a repressible protease that is catalytically active. For HCV NS3, the catalytic triad comprises His-57, Asp-81, and Ser-139. In regard to a repressible protease, “catalytically active” refers to the ability to cleave at a protease cleavage site. In some embodiments of any of the aspects, the catalytically active repressible protease can be any repressible protease as described further herein that maintains the catalytic triad, i.e., comprises no non-synonymous substitutions at His-57, Asp-81, and/or Ser-139.

In some embodiments of any of the aspects, an iAD polypeptide as described herein comprises a repressible protease that is catalytically inactive, i.e., dead. In regard to a repressible protease, “catalytically inactive” refers to the inability to cleave at a protease cleavage site. Accordingly, a catalytically inactive NS3 protease can comprise a nonsynonymous mutation at any one of His-57, Asp-81, and Ser-139. Non-limiting examples of NS3 inactivating mutations include H57A, D81A, S139A, or any combination thereof. As such, any one of SEQ ID NOs: 208-224 or SEQ ID NOs: 252-260 can comprise a H57A mutation; a D81A mutation; a S139A mutation; any nonsynonymous mutation to His-57, Asp-81, and Ser-139; or any combination thereof. In some embodiments of any of the aspects, any one of SEQ ID NOs: 208-224 or SEQ ID NOs: 252-260 can comprise a S139A mutation. In some embodiments of any of the aspects, a mutation to the catalytic triad does not disrupt other functions of the repressible protease, e.g., binding to a protease inhibitor, and/or binding to a peptide domain.

In some embodiments of any of the aspects, a catalytically-inactive repressible protease of an iAD polypeptide as described herein comprises SEQ ID NOs: 99 or 103, or an amino acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NOs: 99 or 103 that maintains the same functions as SEQ ID NOs: 262 or 263 (e.g., catalytically inactive). In some embodiments of any of the aspects, a catalytically-inactive repressible protease of an iAD polypeptide as described herein comprises SEQ ID NOs: 262 or 263, or an amino acid sequence that is at least 95% identical to the sequence of SEQ ID NOs: 262 or 263 that maintains the same functions as SEQ ID NOs: 262 or 263 (e.g., catalytically inactive, but maintaining functions of the repressible protease, e.g., binding to a protease inhibitor, and/or binding to a peptide domain).

In some embodiments of any of the aspects, a catalytically-inactive repressible protease is encoded by a nucleic acid sequence comprising SEQ ID NOs: 264 or 265 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to SEQ ID NOs: 264 or 265 that maintains the same function, or a codon-optimized version thereof. In some embodiments of any of the aspects, a catalytically-inactive repressible protease is encoded by a nucleic acid sequence comprising SEQ ID NOs: 75, 79 or a sequence that is at least 95% identical to SEQ ID NOs: 264 or 265 that maintains the same function.

SEQ ID NO: 262, NS3 (genotype 1B; S139A), 537 nt; bold text (e.g., nt 409-411 of SEQ ID NO: 75) indicates the conserved S139 residue mutated to alanine, i.e., S139A.

NS3 (genotype 1B; S139A), 537 nt; bold text (e.g., nt 409-411 of SEQ ID
NO: 75) indicates the conserved S139 residue mutated to alanine, i.e., S139A.
SEQ ID NO: 262
ATCACGGCCTACTCCCAACAGACGCGGGGCCTACTTGGTTGCATCATCACTAGCCTCACAGG
CCGGGACAAGAACCAGGTCGAAGGGGAGGTTCAAGTGGTTTCTACCGCAACACAATCTTTC
CTGGCGACCTGCGTCAACGGCGTGTGCTGGACTGTCTACCATGGCGCTGGCTCGAAGACCCT
AGCCGGTCCAAAAGGTCCAATCACCCAAATGTACACCAATGTAGACCAGGACCTCGTCGGC
TGGCAGGCGCCTCCAGGGGCGCGCTCCTTGACACCATGCACCTGTGGCAGCTCGGACCTTTA
CTTGGTCACGAGACATGCTGATGTCATTCCGGTGCGCCGGCGAGGCGACAGCAGGGGAAGT
CTACTCTCCCCCAGGCCCGTCTCCTACCTGAAAGGCTCCGCAGGTGGTCCATTGCTTTGCCCT
TCGGGGCACGCTGTGGGCATCTTCCGGGCTGCTGTGTGCACCCGGGGGGTCGCGAAGGCGGT
GGACTTCGTGCCCGTTGAGTCTATGGAAACTACCATGCGGTCT
NS3 (genotype 1A; S139A), 567 nt; bold dotted underlined text indicates His-57 of the
catalytic triad; italicized double underlined text indicates Asp-81 of the catalytic
triad; bold italicized dotted underlined text indicates Ser-139 of the catalytic triad
mutated to alanine (S139A); zig zag underlined text indicates Asp-168.
SEQ ID NO: 264
GCGCCCATCACGGCGTACGCCCAGCAGACGAGAGGCCTCCTAGGGTGTATAATCACCAGCC
TGACTGGCCGGGACAAAAACCAAGTGGAGGGTGAGGTCCAGATCGTGTCAACTGCTACCCA
AACCTTCCTGGCAACGTGCATCAATGGGGTATGCTGGGCAGTCTAC [symbol] GGGGCCGGAACG
AGGACCATCGCATCACCCAAGGGTCCTGTCATCCAGATGTATACCAATGTGGACCAA [symbol] CT
TGTGGGCTGGCCCGCTCCTCAAGGTTCCCGCTCATTGACACCCTGTACCTGCGGCTCCTCGGA
CCTTTACCTGGTCACGAGGCACGCCGATGTCATTCCCGTGCGCCGGCGAGGTGATAGCAGGG
GTAGCCTGCTTTCGCCCCGGCCCATTTCCTACTTGAAAGGCTCC [symbol] GGGGGTCCGCTGTTGT
GCCCCGCGGGACACGCCGTGGGCCTATTCAGGGCCGCGGTGTGCACCCGTGGAGTGGCTAA
AGCGGTG [symbol] TTTATCCCTGTGGAGAACCTAGAGACAACCATGAGATCCCCGGTGTTCACGG
ACAACTCCTCT
NS3 (genotype 1B; S139A), 179 aa; bold text (e.g., nt 409-411 of SEQ ID
NO: 262) indicates S139A.
SEQ ID NO: 263
ITAYSQQTRGLLGCIITSLTGRDKNQVEGEVQVVSTATQSFLATCVNGVCWTVYHGAGSKTLAG
PKGPITQMYTNVDQDLVGWQAPPGARSLTPCTCGSSDLYLVTRHADVIPVRRRGDSRGSLLSPRP
VSYLKGSAGGPLLCPSGHAVGIFRAAVCTRGVAKAVDFVPVESMETTMRS
NS3 (genotype 1A; S139A), 189 aa; bold dotted underlined text indicates His-57 of the
catalytic triad; italicized double underlined text (e.g., nt 241-243 of SEQ ID NO:
264) indicates Asp-81 of the catalytic triad; bold italicized dotted underlined text
(e.g., nt 415-417 of SEQ ID NO: 264) indicates Ser-139 of the catalytic triad mutated to
alanine (S139A); zig zag underlined text (e.g., nt 502-504 of SEQ ID NO: 264) indicates
Asp-168.
SEQ ID NO: 265
APITAYAQQTRGLLGCIITSLTGRDKNQVEGEVQIVSTATQTFLATCINGVCWAVY [symbol] GAGTRTIA
SPKGPVIQMYTNVDQ [symbol] LVGWPAPQGSRSLTPCTCGSSDLYLVTRHADVIPVRRRGDSRGSLLSPR
PISYLKGS [symbol] GGPLLCPAGHAVGLFRAAVCTRGVAKAV [symbol] FIPVENLETTMRSPVFTDNSS

In some embodiments of any of the aspects, the binding between the repressible protease and its cognate peptide domain can be disrupted by an inducer, such as a protease inhibitor. In some embodiments of any of the aspects, an iAD polypeptide as described herein is in combination with a protease inhibitor. As used herein, “in combination with” refers to two or more substances being present in the same formulation in any molecular or physical arrangement, e.g., in an admixture, in a solution, in a mixture, in a suspension, in a colloid, in an emulsion. The formulation can be a homogeneous or heterogeneous mixture. In some embodiments of any of the aspects, the active compound(s) can be comprised by a superstructure, e.g., nanoparticles, liposomes, vectors, cells, scaffolds, or the like, said superstructure is which in solution, mixture, admixture, suspension, etc., with the iAD polypeptide or iAD polypeptide system. In some embodiments of any of the aspects, the iAD polypeptide is bound to a protease inhibitor bound to the repressible protease. In some embodiments of any of the aspects, the iAD polypeptide is bound specifically to a protease inhibitor bound to the repressible protease.

In some embodiments of any of the aspects, the iAD polypeptide is in combination with 1, 2, 3, 4, 5, or more protease inhibitors. In some embodiments of any of the aspects, the iAD polypeptide is in combination with one protease inhibitor. In embodiments comprising multiple protease inhibitors, the multiple protease inhibitors can be different individual protease inhibitors or multiple copies of the same protease inhibitor, or a combination of the foregoing.

In some embodiments of any of the aspects, the protease inhibitor is grazoprevir (abbreviated as GZV or GZP; see e.g., PubChem CID: 44603531). In some embodiments of any of the aspects, the protease inhibitor is danoprevir (DNV; see e.g., PubChem CID: 11285588). In some embodiments of any of the aspects, the protease inhibitor is an approved NS3 protease inhibitor, such as but not limited to grazoprevir, danoprevir, simeprevir, asunaprevir, ciluprevir, boceprevir, sovaprevir, paritaprevir, ombitasvir, paritaprevir, ritonavir, dasabuvir, and telaprevir. Additional non-limiting examples of NS3 protease inhibitors are listed in Table 9 (see e.g., McCauley and Rudd, Hepatitis C virus NS3/4a protease inhibitors, Current Opinion in Pharmacology 2016, 30:84-92; the content of which is incorporated herein by reference in its entirety).

TABLE 9
Exemplary NS3/NS4A protease inhibitors

| Description or Name(s) | Structure |
|---|---|
| The N-terminal hexapeptide product of substrate cleavage (e.g., DDIVPC-OH (SEQ ID NO: 404)) | 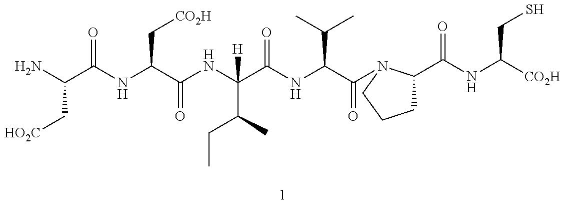 [SMILES: CC[C@H](C)[C@H](NC(=O)[C@H](CC(=O)O)NC(=O)[C@@H](N)CC(=O)O)C(=O)N[C@H](C(=O)N1CCC[C@H]1C(=O)N[C@@H](CS)C(=O)O)C(C)C] |
| One of the products of cleavage of the NS4a-NS4b peptide (e.g., Ac-DEMEEC- OH (SEQ ID NO: 405)) | 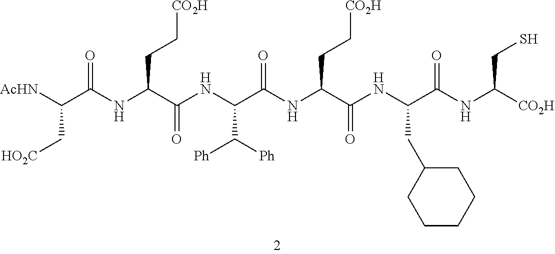 [SMILES: CC(=O)N[C@@H](CC(=O)O)C(=O)N[C@@H](CCC(=O)O)C(=O)N[C@H](C(=O)N[C@@H](CCC(=O)O)C(=O)N[C@@H](CC1CCCCC1)C(=O)N[C@@H](CS)C(=O)O)C(c1ccccc1)c1ccccc1] |
| VICTRELIS ™ boceprevir SCH503034 | 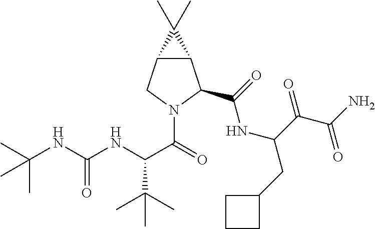 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C] |
| INCIVEK ™, INCIVIO ™, telaprevir, VX-950 | 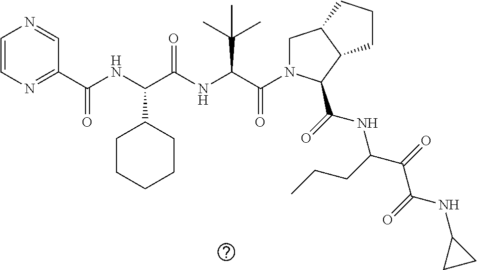 [SMILES: CCCC(NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1] |
| Ciluprevir; BILN-2061 | 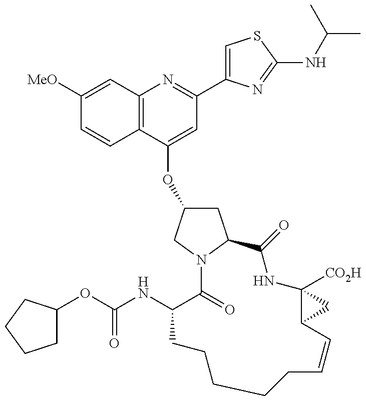 [SMILES: COc1ccc2c(O[C@@H]3C[C@H]4C(=O)N[C@]5(C(=O)O)C[C@H]5/C=C\CCCCC[C@H](NC(=O)OC5CCCC5)C(=O)N4C3)cc(-c3csc(NC(C)C)n3)nc2c1] |
| BMS-605339 | 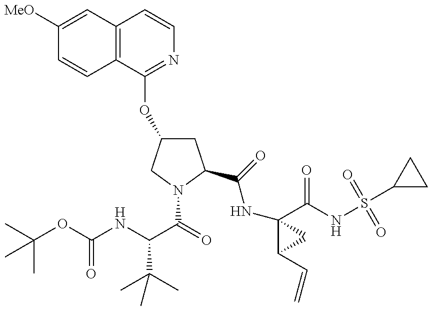 |
| MK-4519 | 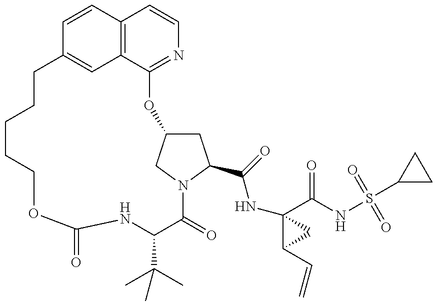 [SMILES: C=C[C@@H]1C[C@]1(NC(=O)[C@@H]1C[C@@H]2CN1C(=O)[C@H](C(C)(C)C)NC(=O)OCCCCCc1ccc3ccnc(c3c1)O2)C(=O)NS(=O)(=O)C1CC1] |
| faldaprevir, BI-201335 | 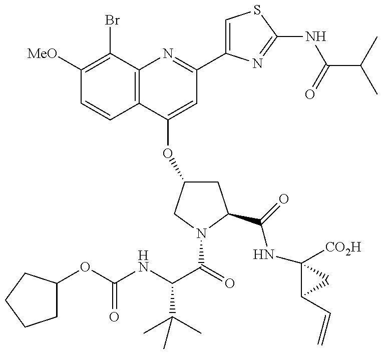 |
| Danoprevir, ITMN-191, R7227 | 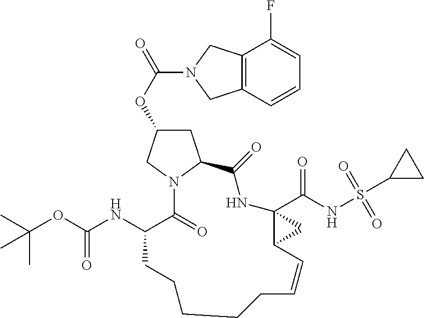 [SMILES: CC(C)(C)OC(=O)N[C@H]1CCCCC/C=C\[C@@H]2C[C@@]2(C(=O)NS(=O)(=O)C2CC2)NC(=O)[C@@H]2C[C@@H](OC(=O)N3Cc4cccc(F)c4C3)CN2C1=O] |
| SUNVEPRA ™, asunaprevir, BMS-650032 | 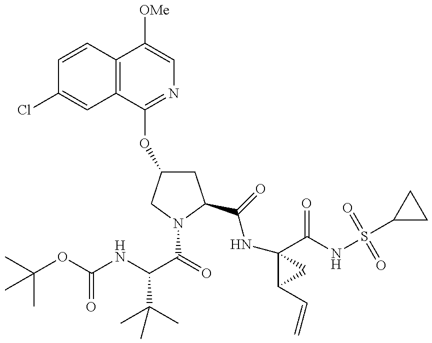 |
| VANIHEP ™, vaniprevir, MK-7009 | 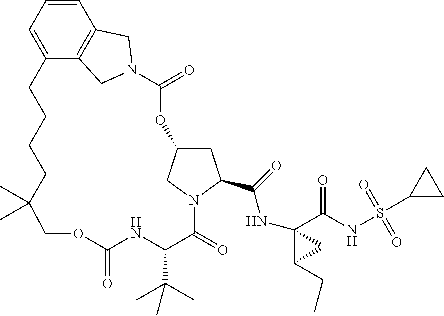 [SMILES: CC[C@@H]1C[C@]1(NC(=O)[C@@H]1C[C@@H]2CN1C(=O)[C@H](C(C)(C)C)NC(=O)OCC(C)(C)CCCCc1cccc3c1CN(C3)C(=O)O2)C(=O)NS(=O)(=O)C1CC1] |
| OLYSIO ™, simeprevir, TMC-435350 | 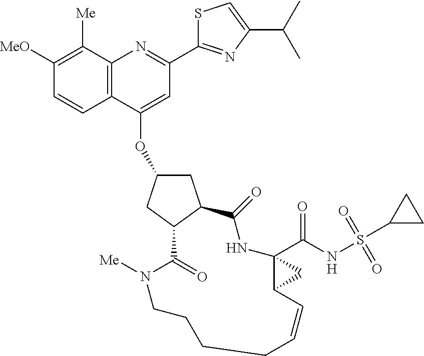 [SMILES: COc1ccc2c(O[C@@H]3C[C@H]4C(=O)N[C@]5(C(=O)NS(=O)(=O)C6CC6)C[C@H]5/C=C\CCCCN(C)C(=O)[C@@H]4C3)cc(-c3nc(C(C)C)cs3)nc2c1C] |
| Sovaprevir, ACH-1625 | 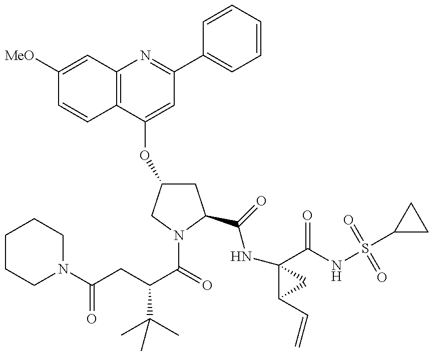 [SMILES: C=C[C@@H]1C[C@]1(NC(=O)[C@@H]1C[C@@H](Oc2cc(-c3ccccc3)nc3cc(OC)ccc23)CN1C(=O)[C@@H](CC(=O)N1CCCCC1)C(C)(C)C)C(=O)NS(=O)(=O)C1CC1] |
| Deldeprevir/neceprevir, ACH-2684 | 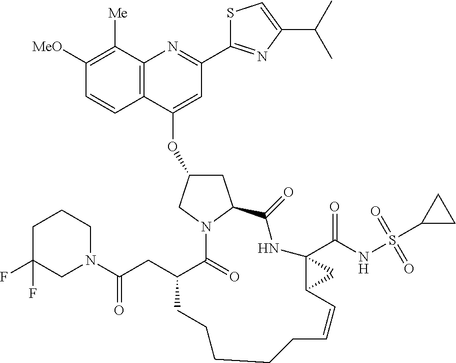 [SMILES: COc1ccc2c(O[C@@H]3C[C@H]4C(=O)N[C@]5(C(=O)NS(=O)(=O)C6CC6)C[C@H]5/C=C\CCCCC[C@H](CC(=O)N5CCCC(F)(F)C5)C(=O)N4C3)cc(-c3nc(C(C)C)cs3)nc2c1C] |
| IDX320 | 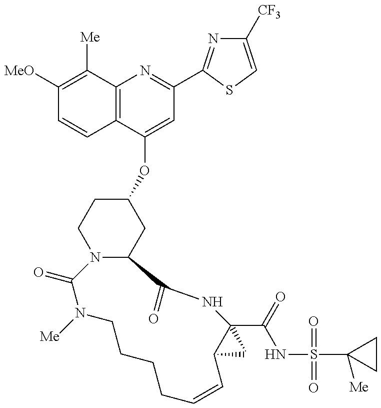 |
| GS-9256 | 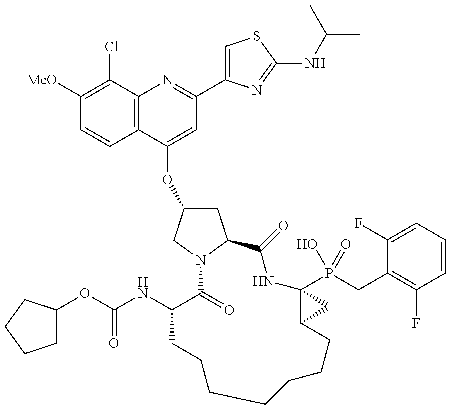 |
| PHX1766 | 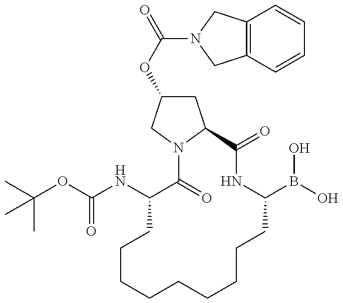 [SMILES: CC(C)(C)OC(=O)N[C@H]1CCCCCCCCC[C@@H](B(O)O)NC(=O)[C@@H]2C[C@@H](OC(=O)N3Cc4ccccc4C3)CN2C1=O] |
| MK-2748 | 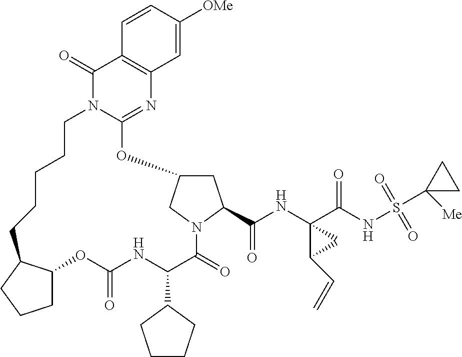 [SMILES: C=C[C@@H]1C[C@]1(NC(=O)[C@@H]1C[C@@H]2CN1C(=O)[C@H](C1CCCC1)NC(=O)O[C@@H]1CCC[C@H]1CCCCCn1c(nc3cc(OC)ccc3c1=O)O2)C(=O)NS(=O)(=O)C1(C)CC1] |
| Vedrorevir, GS-9451, GS- 9451 | 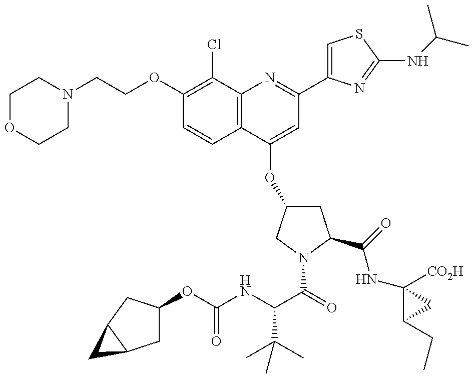 |
| MK-6325 | 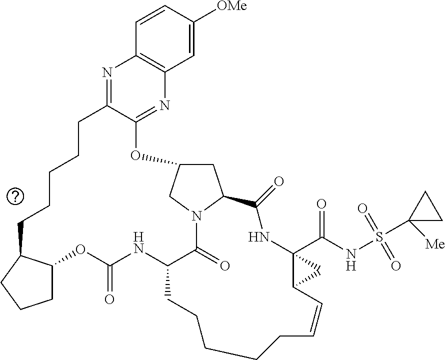 [SMILES: COc1ccc2nc3c(nc2c1)O[C@@H]1C[C@H]2C(=O)N[C@]4(C(=O)NS(=O)(=O)C5(C)CC5)C[C@H]4/C=C\CCCCC[C@H](NC(=O)O[C@@H]4CCC[C@H]4CCCCC3)C(=O)N2C1] |
| MK-8831 | 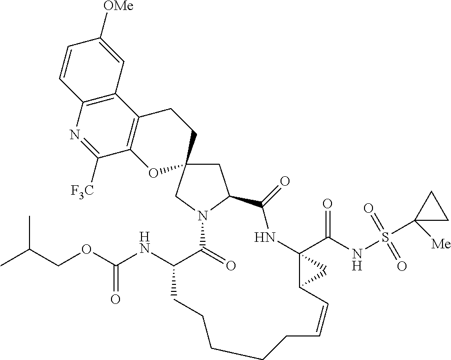 |
| VIKERA PAK ™, paritaprevir, ABT-450 | 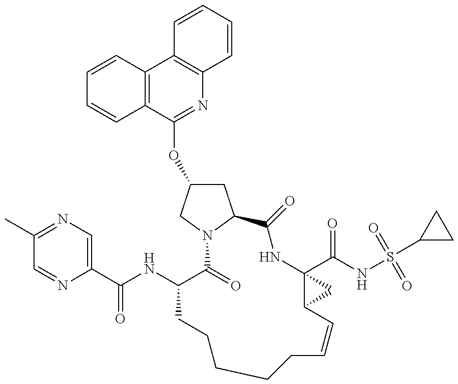 |
| ZEPATIER ™, grazoprevir, MK-5172 | 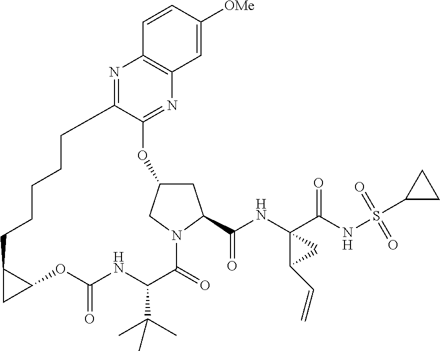 [SMILES: C=C[C@@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)NS(=O)(=O)C1CC1] |
| Glecaprevir, ABT-493 | 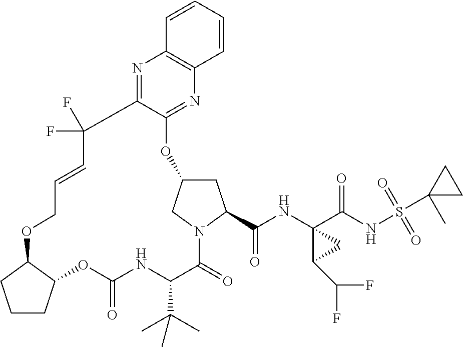 [SMILES: CC(C)(C)[C@@H]1NC(=O)O[C@@H]2CCC[C@H]2OC/C=C/C(F)(F)c2nc3ccccc3nc2O[C@@H]2C[C@@H](C(=O)N[C@]3(C(=O)NS(=O)(=O)C4(C)CC4)C[C@H]3C(F)F)N(C2)C1=O] |
| Voxilaprevir, GS-9857 | 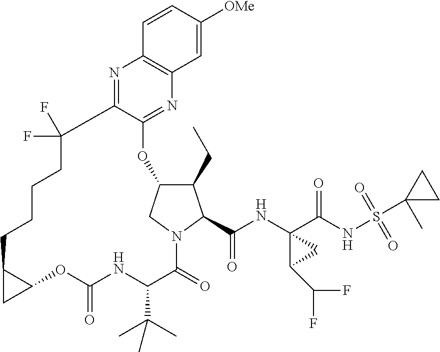 [SMILES: CC[C@@H]1[C@@H]2CN(C(=O)[C@H](C(C)(C)C)NC(=O)O[C@@H]3C[C@H]3CCCCC(F)(F)c3nc4ccc(OC)cc4nc3O2)[C@@H]1C(=O)N[C@]1(C(=O)NS(=O)(=O)C2(C)CC2)C[C@H]1C(F)F] |

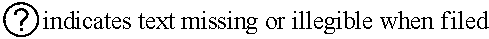

In several aspects, described herein are iAD polypeptides comprising a peptide domain. As used herein, the term “peptide domain” refers to a short polypeptide domain that can specifically bind to a repressible protease as described herein (e.g., NS3 protease). The peptide domain can also be referred to herein as a “protease-binding domain”. In some embodiments of any of the aspects, any peptide that can bind to the repressible protease can be used. In some embodiments of any of the aspects, the peptide domain comprises a protease cleavage site as described herein and is a substrate peptidomimetic. In some embodiments of any of the aspects, the peptide domain is specifically bound by but not cleaved by the repressible protease. In some embodiments of any of the aspects, an iAD polypeptide as described herein (or an iAD polypeptide system collectively) comprises 1, 2, 3, 4, 5, or more peptide domains. In some embodiments of any of the aspects, the iAD polypeptide or system comprises one peptide domain. In embodiments comprising multiple peptide domains, the multiple peptide domains can be different individual peptide domains or multiple copies of the same peptide domain, or a combination of the foregoing.

Table 10 lists non-limiting examples of peptide domains (e.g., for NS3 protease). Such inhibitory peptides cap the active site and bind via a “tyrosine” finger at an alternative NS3-4A site. The peptides are not cleaved due to a combination of geometrical constraints and impairment of the oxyanion hole function. Negligible susceptibility to known (e.g., A156V and R155K) resistance mutations of the NS3-4A protease have been observed. Accordingly, non-limiting examples of peptide domains include: K5-66, K5-66-A, K5-66-B, K6-10, K6-10A, K6-10B K5-66-R, CP5-46, CP5-46-4D5E, CP5-46-A, CP5- 46A-4D5E, Ant-CP5-46A-4D5E, and apo NS3a reader (ANR) peptides (see e.g., Kugler et al., High Affinity Peptide Inhibitors of the Hepatitis C Virus NS3-4A Protease Refractory to Common Resistant Mutants, J Biol Chem. 2012 Nov. 9; 287(46): 39224-39232; Cunningham-Bryant et al., J Am Chem Soc. 2019 Feb. 27; 141(8):3352-3355).

TABLE 10
Exemplary Peptide Domains

| SEQ | | |
|---|---|---|
| ID | | |
| NO: | Peptide | Sequence |
| 266 | K5-66 | GELGRLVYLLDGPGYDPIHC |
| | | SLAYGDASTLVVF |
| 267 | K5-66-A | GELGRLVYLLDGPGYDPI |
| 268 | K5-66-B | HCSLAYGDASTLVVF |
| 269 | K6-10 | GELGRPVYVLGDPGYYA |
| | | THCIYATTNDALIFSV |
| 270 | K6-10-A | GELGRPVYVLGDPGYYAT |
| 271 | K6-10-B | HCIYATTNDALIFSV |
| 272 | K5-66-R | GELGRIPSDTYDLAVGA |
| | | LHCPFYLVSGLVYLDG |
| 273 | CP5-46 | GELGRLVYLLDGPGYDP |
| | | IHCDVVTRGGSHLFNF |
| 274 | CP5-46- | GELDELVYLLDGPGYDP |
| | 4D5E | IHCDVVTRGGSHLFNF |
| 275 | CP5-46-A | GELGRLVYLLDGPGYDPIHCD |
| 276 | CP5-46A- | GELDELVYLLDGPGYDPIHS |
| | 4D5E | |
| 277 | Ant-CP5- | RQIK IWFQNRRMKWKKGEL |
| | 46A-4D5E | DELVYLLDGPGYDPIHS |
| 261 | ANR | GELDELVYLLDGPGYDPIHSD |

In some embodiments of any of the aspects, the peptide domain of an iAD polypeptide as described herein comprises SEQ ID NOs: 261, 266-277, or an amino acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 261, 266-277, that maintains the same functions as one of SEQ ID NOs: 261, 266-277 (e.g., binding to a repressible protease). In some embodiments of any of the aspects, the peptide domain of an iAD polypeptide as described herein comprises SEQ ID NOs: 261, 266-277, or an amino acid sequence that is at least 95% identical to the sequence of one of SEQ ID NOs: 261, 266-277, that maintains the same functions as one of SEQ ID NOs: 261, 266-277.

In some embodiments of any of the aspects, the peptide domain of an iAD polypeptide as described herein is encoded by a nucleic acid sequence comprising SEQ ID NO: 278 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to SEQ ID NO: 278 that maintains the same function or a codon-optimized version of SEQ ID NO: 278. In some embodiments of any of the aspects, the peptide domain of an iAD polypeptide as described herein is encoded by a nucleic acid sequence comprising SEQ ID NO: 278 or a sequence that is at least 95% identical to SEQ ID NO: 278 that maintains the same function.

CP5-46-5D5E, 99 nt
SEQ ID NO: 278
GGAGAACTTGATGAATTGGTATACTTACTAG
ATGGGCCAGGTTATGACCCTATACATTGCGA
TGTAGTGACAAGGGGCGGCAGCCACCTTTTC
AATTTT,

In some embodiments of any of the aspects, a peptide domain is specific for a certain genotype of repressible protease. As a non-limiting example, the peptide ANR (e.g., SEQ ID NO: 261) was selected to interact with genotype 1b NS3a (e.g., SEQ ID NO: 211) or an NS3 comprising the following mutations: A7S, E13L, 135V and T42S (e.g., SEQ ID NO: 260). Apo NS3a reader (ANR) forms a basal complex with NS3a-genotype 1b with an affinity of 10 nM, which is disrupted by NS3a-targeting drugs. Accordingly, described herein are iAD systems comprising a peptide domain (e.g., SEQ ID NO: 261, 266-277) and a repressible protease (e.g., SEQ ID NO: 211, 260).

Described herein are iAD polypeptides comprising protease cleavage sites. As used herein, the term “protease cleavage site” refers to a specific sequence or sequence motif recognized by and cleaved by the repressible protease. A cleavage site for a protease includes the specific amino acid sequence or motif recognized by the protease during proteolytic cleavage and typically includes the surrounding one to six amino acids on either side of the scissile bond, which bind to the active site of the protease and are used for recognition as a substrate. In some embodiments of any of the aspects, the protease cleavage site can be any site specifically bound by and cleaved by the repressible protease. In some embodiments of any of the aspects, an iAD polypeptide as described herein comprises 1, 2, 3, 4, 5, or more protease cleavage sites. In some embodiments of any of the aspects, the iAD polypeptide comprises one protease cleavage site. In some embodiments of any of the aspects, the iAD polypeptide comprises two protease cleavage sites. In embodiments comprising multiple protease cleavage sites, the multiple protease cleavage sites can be different individual protease cleavage sites or multiple copies of the same protease cleavage sites, or a combination of the foregoing.

As a non-limiting example, during HCV replication, the NS3-4A serine protease is responsible for the proteolytic cleavage at four junctions of the HCV polyprotein precursor: NS3/NS4A (self-cleavage), NS4A/NS4B, NS4B/NS5A, and NS5A/NS5B. Accordingly, the protease cleavage site of an iAD polypeptide as described herein can be a NS3/NS4A cleavage site, a NS4A/NS4B cleavage site, a NS4B/NS5A cleavage site, or a NS5A/NS5B cleavage site. The protease cleavage site can be a protease cleavage sites from HCV genotype 1, genotype 1a, genotype 1b, genotype 2, genotype 2a, genotype 2b, genotype 3, genotype 3a, genotype 3b, genotype 4, genotype 4a, genotype 4b, genotype 5, genotype 5a, genotype 5b, genotype 6, genotype 6a, genotype 6b, genotype 7, genotype 7a NS4A, or genotype 7b. In some embodiments of any of the aspects, the protease cleavage site can be any known NS3/NS4A protease cleavage site or variant or mutant thereof, e.g., that maintains the same function. In some embodiments of any of the aspects, the NS4A sequence comprises residues 21-31 of the NS4A protein from HCV-H, HCV-1, HCV-J1, HCV-BK, HCV-JK1, HCV-J4, HCV-J, HCV-J6, C14112, HCV-J8, D14114, HCV-Nz11, or HCV-K3a (see e.g., Chao Lin 2006 supra).

In some embodiments of any of the aspects, the protease cleavage site of an iAD polypeptide as described herein comprises SEQ ID NOs: 364-389, or an amino acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 364-389 that maintains the same function.

In some embodiments of any of the aspects, the protease cleavage site of an iAD polypeptide as described herein comprises residues 1-20, 1-19, 1-18, 1-17, 1-16, 1-15, 2-20, 2-19, 2-18, 2-17, 2-16, 2-15, 3-20, 3-19, 3-18, 3-17, 3-16, 3-15, 4-20, 4-19, 4-18, 4-17, 4-16, 4-15, 5-20, 5-19, 5-18, 5-17, 5-16, or 5-15, of any of SEQ ID NOs: 208-224.

NS5A/5B cut site (CC), 10 aa,
SEQ ID NO: 364
EDVVCCHSIY,
NS4A/4B cut site (CS), 14 aa,
SEQ ID NO: 365
LYQEFDEMEECSQH,
N3 cleavage site (NS4A/4B cut site),
SEQ ID NO: 366
DEMEECSQHL,
SEQ ID NO: 367
QEFEDVVPCSMGS,
NS5A/5B cut site,
SEQ ID NO: 368
EDVVCCHSI,
NS4A/4B cut site,
SEQ ID NO: 369
DEMEECSQH,

TABLE 13
Exemplary NS3/NS4A protease cleavage sites
(see e.g., Chao Lin 2006 supra).

| Cleavage | SEQ | | Sequence (cleavage |
|---|---|---|---|
| Site | ID | Genotype | site shown with |
| Type | NO | (Strain) | space) |
| NS3/ | 370 | 1a (HCV-H) | CMSADLEVVT STWVLVGGVL |
| NS4A | | | |
| | 371 | 1b (HCV-BK) | CMSADLEVVT STWVLVGGVL |
| | 372 | 2a (HCV-J6) | CMQADLEVMT STWVLAGGVL |
| | 373 | 2b (HCV-J8) | CMQADLEIMT SSWVLAGGVL |
| | 374 | 3a (HCV-Nz11) | CMSADLEVTT STWVLLGGVL |
| NS4A/ | 375 | 1a (HCV-H) | YQEFDEMEEC SQHLPYIEQG |
| NS4B | | | |
| | 376 | 1b (HCV-BK) | YQEFDEMEEC ASHLPYIEQG |
| | 377 | 2a (HCV-J6) | YEAFDEMEEC ASRAALIEEG |
| | 378 | 2b (HCV-J8) | YEAFDEMEEC ASKAALIEEG |
| | 379 | 3a (HCV-Nz11) | YQQYDEMEEC SQAAPYIEQA |
| NS4B/ | 380 | 1a (HCV-H) | WISSECTTPC SGSWLRDVWD |
| NS5A | | | |
| | 381 | 1b (HCV-BK) | WINEDCSTPC SGSWLRDVWD |
| | 382 | 2a (HCV-J6) | WITEDCPIPC SGSWLRDVWD |
| | 383 | 2b (HCV-J8) | WITEDCPVPC SGSWLQDIWD |
| | 384 | 3a (HCV-Nz11) | WINEDYPSPC SDDWLRTIWD |
| NS5A/ | 385 | 1a (HCV-H) | GADTEDVVCC SMSYSWTGAL |
| NS5B | | | |
| | 386 | 1b (HCV-BK) | EEASEDVVCC SMSYTWTGAL |
| | 387 | 2a (HCV-J6) | SEEDDSVVCC SMSYSWTGAL |
| | 388 | 2b (HCV-J8) | SDQEDSVICC SMSYSWTGAL |
| | 389 | 3a (HCV-Nz11) | DSEEQSVVCC SMSYSWTGAL |

In some embodiments of any of the aspects, an iAD polypeptide as described herein comprises two protease cleavage sites, with one N-terminal of the NS3-NS4A complex, and the other C-terminal of the NS3-NS4A complex (see e.g., Table 14). In some embodiments of any of the aspects, the two protease cleavage sites can be the same cleavage sites or different cleavage sites.

TABLE 14
Exemplary Protease Cleavage Site Combinations.

| N | 3/4A | | | | 4A/4B | | | |
|---|---|---|---|---|---|---|---|---|
| C | 3/4A | 4A/4B | 4B/5A | 5A/5B | 3/4A | 4A/4B | 4B/5A | 5A/5B |
| N | 4B/5A | | | | 5A/5B | | | |
| C | 3/4A | 4A/4B | 4B/5A | 5A/5B | 3/4A | 4A/4B | 4B/5A | 5A/5B |

“N” indicates N-terminal of the NS3-NS4A complex.
“C” indicates C-terminal of the NS3-NS4A complex.
“3/4A” indicates the NS3/NS4A cleavage site.
“4A/4B” indicates the NS4A/NS4B cleavage site.
“4B/5A” indicates the NS4B/NS5A cleavage site.
“5A/5B” indicates the NS5A/NS5B cleavage site.

In some embodiments of any of the aspects, an iAD polypeptide as described herein comprise any known genotypes, variants, or mutants of NS3/NS4A, NS4A/NS4B, NS4B/NS5A, and NS5A/NS5B cleavage sites. In one embodiment, the two protease cleavage sites are selected from selected from the same genotype as each other.

D. Exemplary iADAR fusion proteins.

TABLE 5
Exemplary fusion proteins are
disclosed in the following table:

| | | | Corresponding |
|---|---|---|---|
| | SEQ | | sequence |
| | ID | | including |
| | NO: | | TagBFP |
| MCP- | 150 | MASNFTQFVLVDNGGTGDVTVAPSNFANGI | 17 |
| linker- | | AEWISSNSRSQAYKVTCSVRQSSAQNRKYT | |
| ADAR2- | | IKVEVPKGAWRSYLNMELTIPIFATNSDCE | |
| DDN- | | LIVKAMQGLLKDGNPIPSAIAANSGIYGGS | |
| Bad(F)- | | GSGAGSGSPAGGGAPGSGGGSQLHLPQVLA | |
| ADAR2(E | | DAVSRLVLGKFGDLTDNFSSPHARRKVLAG | |
| 488Q)- | | VVMTTGTDVKDAKVISVSTGTKCINGEYMS | |
| DDC | | DRGLALNDCHAEIISRRSLLRFLYTQLELY | |
| | | LNNKDDQKRSIFQKSERGGFRLKENVQFHL | |
| | | YISTSPCGDARIFSPHEPILEEPAASGSGT | |
| | | GAPPNLWAAQRYGRELRRMSDEFVDRHPNR | |
| | | KARGQLRTKIESGQGTIPVRSNASIQTWDG | |
| | | VLQGERLLTMSCSDKIARWNVVGIQGSLLS | |
| | | IFVEPIYFSSIILGSLYHGDHLSRAMYQRI | |
| | | SNIEDLPPLYTLNKPLLSGISNAEARQPGK | |
| | | APNFSVNWTVGDSAIEVINATTGKDELGRA | |
| | | SRLCKHALYCRWMRVHGKVPSHLLRSKITK | |
| | | PNVYHESKLAAKEYQAAKARLFTAFIKAGL | |
| | | GAWVEKPTEQDQFSLT | |
| MCP- | 151 | MASNFTQFVLVDNGGTGDVTVAPSNFANGI | 18 |
| linker- | | AEWISSNSRSQAYKVTCSVRQSSAQNRKYT | |
| ADAR2- | | IKVEVPKGAWRSYLNMELTIPIFATNSDCE | |
| DDN- | | LIVKAMQGLLKDGNPIPSAIAANSGIYGGS | |
| Bad(F)- | | GSGAGSGSPAGGGAPGSGGGSQLHLPQVLA | |
| ADAR2(E | | DAVSRLVLGKFGDLTDNFSSPHARRKVLAG | |
| 488Q)- | | VVMTTGTDVKDAKVISVSTGTKCINGEYMS | |
| DDC-Bcl- | | DRGLALNDCHAEIISRRSLLRFLYTQLELY | |
| XL-T | | LNNKDDQKRSIFQKSERGGFRLKENVQFHL | |
| | | YISTSPCGDARIFSPHEPILEEPAASGSGT | |
| | | GAPPNLWAAQRYGRELRRMSDEFVDRHPNR | |
| | | KARGQLRTKIESGQGTIPVRSNASIQTWDG | |
| | | VLQGERLLTMSCSDKIARWNVVGIQGSLLS | |
| | | IFVEPIYFSSIILGSLYHGDHLSRAMYQRI | |
| | | SNIEDLPPLYTLNKPLLSGISNAEARQPGK | |
| | | APNFSVNWTVGDSAIEVINATTGKDELGRA | |
| | | SRLCKHALYCRWMRVHGKVPSHLLRSKITK | |
| | | PNVYHESKLAAKEYQAAKARLFTAFIKAGL | |
| | | GAWVEKPTEQDQFSLTGSAAGGSGGSAAAS | |
| | | SNRELVVDFLSYKLSQKGYSWSQFSDVEEN | |
| | | RTEAPEGTESEMETPSAINGNPSWHLADSP | |
| | | AVNGATGHSSSLDAREVIPMAAVKQALREA | |
| | | GDEFELRYRRAFSDLTSQLHITPGTAYQSF | |
| | | EQVVNELFRDGVNWGRIVAFFSFGGALCVE | |
| | | SVDKEMQVLVSRIAAWMATYLNDHLEPWIQ | |
| | | ENGGWDTFVELYGNN | |
| MCP- | 152 | MASNFTQFVLVDNGGTGDVTVAPSNFANGI | 20 |
| linker- | | AEWISSNSRSQAYKVTCSVRQSSAQNRKYT | |
| ADAR2- | | IKVEVPKGAWRSYLNMELTIPIFATNSDCE | |
| DDN-Bim- | | LIVKAMQGLLKDGNPIPSAIAANSGIYGGS | |
| ADAR2(E | | GSGAGSGSPAGGGAPGSGGGSQLHLPQVLA | |
| 488Q)- | | DAVSRLVLGKFGDLTDNFSSPHARRKVLAG | |
| DDC | | VVMTTGTDVKDAKVISVSTGTKCINGEYMS | |
| | | DRGLALNDCHAEIISRRSLLRFLYTQLELY | |
| | | LNNKDDQKRSIFQKSERGGFRLKENVQFHL | |
| | | YISTSPCGDARIFSPHEPILEEPAASGSGS | |
| | | GDMRPEIWIAQELRRIGDEFNAYYARRTGD | |
| | | RHPNRKARGQLRTKIESGQGTIPVRSNASI | |
| | | QTWDGVLQGERLLTMSCSDKIARWNVVGIQ | |
| | | GSLLSIFVEPIYFSSIILGSLYHGDHLSRA | |
| | | MYQRISNIEDLPPLYTLNKPLLSGISNAEA | |
| | | RQPGKAPNFSVNWTVGDSAIEVINATTGKD | |
| | | ELGRASRLCKHALYCRWMRVHGKVPSHLLR | |
| | | SKITKPNVYHESKLAAKEYQAAKARLFTAF | |
| | | IKAGLGAWVEKPTEQDQFSLT | |
| MCP- | 153 | MASNFTQFVLVDNGGTGDVTVAPSNFANGI | 22 |
| linker- | | AEWISSNSRSQAYKVTCSVRQSSAQNRKYT | |
| ADAR2- | | IKVEVPKGAWRSYLNMELTIPIFATNSDCE | |
| DDN- | | LIVKAMQGLLKDGNPIPSAIAANSGIYGGS | |
| MS1(A)- | | GSGAGSGSPAGGGAPGSGGGSQLHLPQVLA | |
| ADAR2(E | | DAVSRLVLGKFGDLTDNFSSPHARRKVLAG | |
| 488Q)- | | VVMTTGTDVKDAKVISVSTGTKCINGEYMS | |
| DDC | | DRGLALNDCHAEIISRRSLLRFLYTQLELY | |
| | | LNNKDDQKRSIFQKSERGGFRLKENVQFHL | |
| | | YISTSPCGDARIFSPHEPILEEPAASGGSG | |
| | | GSGRPEIWMTQGLRRLGDEANAYYARRTGD | |
| | | RHPNRKARGQLRTKIESGQGTIPVRSNASI | |
| | | QTWDGVLQGERLLTMSCSDKIARWNVVGIQ | |
| | | GSLLSIFVEPIYFSSIILGSLYHGDHLSRA | |
| | | MYQRISNIEDLPPLYTLNKPLLSGISNAEA | |
| | | RQPGKAPNFSVNWTVGDSAIEVINATTGKD | |
| | | ELGRASRLCKHALYCRWMRVHGKVPSHLLR | |
| | | SKITKPNVYHESKLAAKEYQAAKARLFTAF | |
| | | IKAGLGAWVEKPTEQDQFSLT | |
| MCP- | 154 | MASNFTQFVLVDNGGTGDVTVAPSNFANGI | 23 |
| linker- | | AEWISSNSRSQAYKVTCSVRQSSAQNRKYT | |
| ADAR2- | | IKVEVPKGAWRSYLNMELTIPIFATNSDCE | |
| DDN- | | LIVKAMQGLLKDGNPIPSAIAANSGIYGGS | |
| Bad(L)- | | GSGAGSGSPAGGGAPGSGGGSQLHLPQVLA | |
| ADAR2(E | | DAVSRLVLGKFGDLTDNFSSPHARRKVLAG | |
| 488Q)- | | VVMTTGTDVKDAKVISVSTGTKCINGEYMS | |
| DDC-Bcl- | | DRGLALNDCHAEIISRRSLLRFLYTQLELY | |
| xL | | LNNKDDQKRSIFQKSERGGFRLKENVQFHL | |
| | | YISTSPCGDARIFSPHEPILEEPAASGSGT | |
| | | GAPPNLWAAQRYGRELRRMSDELVDRHPNR | |
| | | KARGQLRTKIESGQGTIPVRSNASIQTWDG | |
| | | VLQGERLLTMSCSDKIARWNVVGIQGSLLS | |
| | | IFVEPIYFSSIILGSLYHGDHLSRAMYQRI | |
| | | SNIEDLPPLYTLNKPLLSGISNAEARQPGK | |
| | | APNFSVNWTVGDSAIEVINATTGKDELGRA | |
| | | SRLCKHALYCRWMRVHGKVPSHLLRSKITK | |
| | | PNVYHESKLAAKEYQAAKARLFTAFIKAGL | |
| | | GAWVEKPTEQDQFSLTGSAAGGSGGSAAAS | |
| | | SNRELVVDFLSYKLSQKGYSWSQFSDVEEN | |
| | | RTEAPEGTESEMETPSAINGNPSWHLADSP | |
| | | AVNGATGHSSSLDAREVIPMAAVKQALREA | |
| | | GDEFELRYRRAFSDLTSQLHITPGTAYQSF | |
| | | EQVVNELFRDGVNWGRIVAFFSFGGALCVE | |
| | | SVDKEMQVLVSRIAAWMATYLNDHLEPWIQ | |
| | | ENGGWDTFVELYGNN | |
| MCP- | 155 | MASNFTQFVLVDNGGTGDVTVAPSNFANGI | 24 |
| linker- | | AEWISSNSRSQAYKVTCSVRQSSAQNRKYT | |
| ADAR2- | | IKVEVPKGAWRSYLNMELTIPIFATNSDCE | |
| DDN- | | LIVKAMQGLLKDGNPIPSAIAANSGIYGGS | |
| MS1(I)- | | GSGAGSGSPAGGGAPGSGGGSQLHLPQVLA | |
| ADAR2(E | | DAVSRLVLGKFGDLTDNFSSPHARRKVLAG | |
| 488Q)- | | VVMTTGTDVKDAKVISVSTGTKCINGEYMS | |
| DDC-Mcl- | | DRGLALNDCHAEIISRRSLLRFLYTQLELY | |
| 1 | | LNNKDDQKRSIFQKSERGGFRLKENVQFHL | |
| | | YISTSPCGDARIFSPHEPILEEPAASGGSG | |
| | | GSGRPEIWMTQGLRRLGDEINAYYARRTGD | |
| | | RHPNRKARGQLRTKIESGQGTIPVRSNASI | |
| | | QTWDGVLQGERLLTMSCSDKIARWNVVGIQ | |
| | | GSLLSIFVEPIYFSSIILGSLYHGDHLSRA | |
| | | MYQRISNIEDLPPLYTLNKPLLSGISNAEA | |
| | | RQPGKAPNFSVNWTVGDSAIEVINATTGKD | |
| | | ELGRASRLCKHALYCRWMRVHGKVPSHLLR | |
| | | SKITKPNVYHESKLAAKEYQAAKARLFTAF | |
| | | IKAGLGAWVEKPTEQDQFSLTGSGTGGPGD | |
| | | ELYRQSLEIISRYLREQATGAKDTKPMGRS | |
| | | GATSRKALETLRRVGDGVQRNHETAFQGML | |
| | | RKLDIKNEDDVKSLSRVMIHVFSDGVTNWG | |
| | | RIVTLISFGAFVAKHLKTINQESCIEPLAE | |
| | | SITDVLVRTKRDWLVKQRGWDGFVEFFHVE | |
| | | DLEGG | |
| MCP- | 156 | MASNFTQFVLVDNGGTGDVTVAPSNFANGI | 25 |
| linker- | | AEWISSNSRSQAYKVTCSVRQSSAQNRKYT | |
| ADAR2- | | IKVEVPKGAWRSYLNMELTIPIFATNSDCE | |
| DDN- | | LIVKAMQGLLKDGNPIPSAIAANSGIYGGS | |
| ALFA- | | GSGAGSGSPAGGGAPGSGGGSQLHLPQVLA | |
| ADAR2(E | | DAVSRLVLGKFGDLTDNFSSPHARRKVLAG | |
| 488Q)- | | VVMTTGTDVKDAKVISVSTGTKCINGEYMS | |
| DDC | | DRGLALNDCHAEIISRRSLLRFLYTQLELY | |
| | | LNNKDDQKRSIFQKSERGGFRLKENVQFHL | |
| | | YISTSPCGDARIFSPHEPILEEPAASPSRL | |
| | | EEELRRRLTEPTGDRHPNRKARGQLRTKIE | |
| | | SGQGTIPVRSNASIQTWDGVLQGERLLTMS | |
| | | CSDKIARWNVVGIQGSLLSIFVEPIYFSSI | |
| | | ILGSLYHGDHLSRAMYQRISNIEDLPPLYT | |
| | | LNKPLLSGISNAEARQPGKAPNFSVNWTVG | |
| | | DSAIEVINATTGKDELGRASRLCKHALYCR | |
| | | WMRVHGKVPSHLLRSKITKPNVYHESKLAA | |
| | | KEYQAAKARLFTAFIKAGLGAWVEKPTEQD | |
| | | QFSLT | |
| MCP- | 157 | MASNFTQFVLVDNGGTGDVTVAPSNFANGI | 26 |
| linker- | | AEWISSNSRSQAYKVTCSVRQSSAQNRKYT | |
| ADAR2- | | IKVEVPKGAWRSYLNMELTIPIFATNSDCE | |
| DDN- | | LIVKAMQGLLKDGNPIPSAIAANSGIYGGS | |
| ALFA- | | GSGAGSGSPAGGGAPGSGGGSQLHLPQVLA | |
| ADAR2(E | | DAVSRLVLGKFGDLTDNFSSPHARRKVLAG | |
| 488Q)- | | VVMTTGTDVKDAKVISVSTGTKCINGEYMS | |
| DDC- | | DRGLALNDCHAEIISRRSLLRFLYTQLELY | |
| NbALFA | | LNNKDDQKRSIFQKSERGGFRLKENVQFHL | |
| | | YISTSPCGDARIFSPHEPILEEPAASPSRL | |
| | | EEELRRRLTEPTGDRHPNRKARGQLRTKIE | |
| | | SGQGTIPVRSNASIQTWDGVLQGERLLTMS | |
| | | CSDKIARWNVVGIQGSLLSIFVEPIYFSSI | |
| | | ILGSLYHGDHLSRAMYQRISNIEDLPPLYT | |
| | | LNKPLLSGISNAEARQPGKAPNFSVNWTVG | |
| | | DSAIEVINATTGKDELGRASRLCKHALYCR | |
| | | WMRVHGKVPSHLLRSKITKPNVYHESKLAA | |
| | | KEYQAAKARLFTAFIKAGLGAWVEKPTEQD | |
| | | QFSLTGSGGTAEVQLQESGGGLVQPGGSLR | |
| | | LSCTASGVTISALNAMAMGWYRQAPGERRV | |
| | | MVAAVSERGNAMYRESVQGRFTVTRDFTNK | |
| | | MVSLQMDNLKPEDTAVYYCHVLEDRVDSFH | |
| | | DYWGQGTQVTVSS | |
| MCP- | 158 | MASNFTQFVLVDNGGTGDVTVAPSNFANGI | 27 |
| linker- | | AEWISSNSRSQAYKVTCSVRQSSAQNRKYT | |
| ADAR2- | | IKVEVPKGAWRSYLNMELTIPIFATNSDCE | |
| DDN- | | LIVKAMQGLLKDGNPIPSAIAANSGIYGGS | |
| ALFA-PE- | | GSGAGSGSPAGGGAPGSGGGSQLHLPQVLA | |
| ADAR2(E | | DAVSRLVLGKFGDLTDNFSSPHARRKVLAG | |
| 488Q)- | | VVMTTGTDVKDAKVISVSTGTKCINGEYMS | |
| DDC | | DRGLALNDCHAEIISRRSLLRFLYTQLELY | |
| | | LNNKDDQKRSIFQKSERGGFRLKENVQFHL | |
| | | YISTSPCGDARIFSPHEPILEEPAASGSGP | |
| | | GRLEEELRRRLSPGTGDRHPNRKARGQLRT | |
| | | KIESGQGTIPVRSNASIQTWDGVLQGERLL | |
| | | TMSCSDKIARWNVVGIQGSLLSIFVEPIYF | |
| | | SSIILGSLYHGDHLSRAMYQRISNIEDLPP | |
| | | LYTLNKPLLSGISNAEARQPGKAPNFSVNW | |
| | | TVGDSAIEVINATTGKDELGRASRLCKHAL | |
| | | YCRWMRVHGKVPSHLLRSKITKPNVYHESK | |
| | | LAAKEYQAAKARLFTAFIKAGLGAWVEKPT | |
| | | EQDQFSLT | |
| MCP- | 159 | MASNFTQFVLVDNGGTGDVTVAPSNFANGI | 28 |
| linker- | | AEWISSNSRSQAYKVTCSVRQSSAQNRKYT | |
| ADAR2- | | IKVEVPKGAWRSYLNMELTIPIFATNSDCE | |
| DDN- | | LIVKAMQGLLKDGNPIPSAIAANSGIYGGS | |
| ALFA-PE- | | GSGAGSGSPAGGGAPGSGGGSQLHLPQVLA | |
| ADAR2(E | | DAVSRLVLGKFGDLTDNFSSPHARRKVLAG | |
| 488Q)- | | VVMTTGTDVKDAKVISVSTGTKCINGEYMS | |
| DDC- | | DRGLALNDCHAEIISRRSLLRFLYTQLELY | |
| NbALFA | | LNNKDDQKRSIFQKSERGGFRLKENVQFHL | |
| | | YISTSPCGDARIFSPHEPILEEPAASGSGP | |
| | | GRLEEELRRRLSPGTGDRHPNRKARGQLRT | |
| | | KIESGQGTIPVRSNASIQTWDGVLQGERLL | |
| | | TMSCSDKIARWNVVGIQGSLLSIFVEPIYF | |
| | | SSIILGSLYHGDHLSRAMYQRISNIEDLPP | |
| | | LYTLNKPLLSGISNAEARQPGKAPNFSVNW | |
| | | TVGDSAIEVINATTGKDELGRASRLCKHAL | |
| | | YCRWMRVHGKVPSHLLRSKITKPNVYHESK | |
| | | LAAKEYQAAKARLFTAFIKAGLGAWVEKPT | |
| | | EQDQFSLTGSGGTAEVQLQESGGGLVQPGG | |
| | | SLRLSCTASGVTISALNAMAMGWYRQAPGE | |
| | | RRVMVAAVSERGNAMYRESVQGRFTVTRDF | |
| | | TNKMVSLQMDNLKPEDTAVYYCHVLEDRVD | |
| | | SFHDYWGQGTQVTVSS | |
| MCP- | 160 | MASNFTQFVLVDNGGTGDVTVAPSNFANGI | 34 |
| linker- | | AEWISSNSRSQAYKVTCSVRQSSAQNRKYT | |
| ADAR2- | | IKVEVPKGAWRSYLNMELTIPIFATNSDCE | |
| DDN- | | LIVKAMQGLLKDGNPIPSAIAANSGIYGGS | |
| SpyTag- | | GSGAGSGSPAGGGAPGSGGGSQLHLPQVLA | |
| ADAR2(E | | DAVSRLVLGKFGDLTDNFSSPHARRKVLAG | |
| 488Q)- | | VVMTTGTDVKDAKVISVSTGTKCINGEYMS | |
| DDC -- | | DRGLALNDCHAEIISRRSLLRFLYTQLELY | |
| P2A-T2A- | | LNNKDDQKRSIFQKSERGGFRLKENVQFHL | |
| SpyCatcher | | YISTSPCGDARIFSPHEPILEEPAASGGSG | |
| | | AHIVMVDAYKPTKGTGDRHPNRKARGQLRT | |
| | | KIESGQGTIPVRSNASIQTWDGVLQGERLL | |
| | | TMSCSDKIARWNVVGIQGSLLSIFVEPIYF | |
| | | SSIILGSLYHGDHLSRAMYQRISNIEDLPP | |
| | | LYTLNKPLLSGISNAEARQPGKAPNFSVNW | |
| | | TVGDSAIEVINATTGKDELGRASRLCKHAL | |
| | | YCRWMRVHGKVPSHLLRSKITKPNVYHESK | |
| | | LAAKEYQAAKARLFTAFIKAGLGAWVEKPT | |
| | | EQDQFSLTGSGSTSATNFSLLKQAGDVEEN | |
| | | PGPGGSEGRGSLLTCGDVEENPGPGTSGGA | |
| | | MVDTLSGLSSEQGQSGDMTIEEDSATHIKF | |
| | | SKRDEDGKELAGATMELRDSSGKTISTWIS | |
| | | DGQVKDFYLYPGKYTFVETAAPDGYEVATA | |
| | | ITFTVNEQGQVTVNGKATKGDAHIG* | |
| MCP- | 161 | MASNFTQFVLVDNGGTGDVTVAPSNFANGI | 35 |
| linker- | | AEWISSNSRSQAYKVTCSVRQSSAQNRKYT | |
| ADAR2- | | IKVEVPKGAWRSYLNMELTIPIFATNSDCE | |
| DDN- | | LIVKAMQGLLKDGNPIPSAIAANSGIYGGS | |
| Spy Tag- | | GSGAGSGSPAGGGAPGSGGGSQLHLPQVLA | |
| ADAR2(E | | DAVSRLVLGKFGDLTDNFSSPHARRKVLAG | |
| 488Q)- | | VVMTTGTDVKDAKVISVSTGTKCINGEYMS | |
| DDC- | | DRGLALNDCHAEIISRRSLLRFLYTQLELY | |
| TEVcs- | | LNNKDDQKRSIFQKSERGGFRLKENVQFHL | |
| SpyCatcher | | YISTSPCGDARIFSPHEPILEEPAASGGSG | |
| | | AHIVMVDAYKPTKGTGDRHPNRKARGQLRT | |
| | | KIESGQGTIPVRSNASIQTWDGVLQGERLL | |
| | | TMSCSDKIARWNVVGIQGSLLSIFVEPIYF | |
| | | SSIILGSLYHGDHLSRAMYQRISNIEDLPP | |
| | | LYTLNKPLLSGISNAEARQPGKAPNFSVNW | |
| | | TVGDSAIEVINATTGKDELGRASRLCKHAL | |
| | | YCRWMRVHGKVPSHLLRSKITKPNVYHESK | |
| | | LAAKEYQAAKARLFTAFIKAGLGAWVEKPT | |
| | | EQDQFSLTGSGGTENLYFQSGTSGGAMVDT | |
| | | LSGLSSEQGQSGDMTIEEDSATHIKFSKRD | |
| | | EDGKELAGATMELRDSSGKTISTWISDGQV | |
| | | KDFYLYPGKYTFVETAAPDGYEVATAITFT | |
| | | VNEQGQVTVNGKATKGDAHI | |
| MCP- | 162 | MASNFTQFVLVDNGGTGDVTVAPSNFANGI | 37 |
| linker- | | AEWISSNSRSQAYKVTCSVRQSSAQNRKYT | |
| ADAR2- | | IKVEVPKGAWRSYLNMELTIPIFATNSDCE | |
| DDN- | | LIVKAMQGLLKDGNPIPSAIAANSGIYGGS | |
| Bad(L)- | | GSGAGSGSPAGGGAPGSGGGSQLHLPQVLA | |
| ADAR2(E | | DAVSRLVLGKFGDLTDNFSSPHARRKVLAG | |
| 488Q)- | | VVMTTGTDVKDAKVISVSTGTKCINGEYMS | |
| DDC- | | DRGLALNDCHAEIISRRSLLRFLYTQLELY | |
| PhoCl-Bcl- | | LNNKDDQKRSIFQKSERGGFRLKENVQFHL | |
| XL | | YISTSPCGDARIFSPHEPILEEPAASGSGT | |
| | | GAPPNLWAAQRYGRELRRMSDELVDRHPNR | |
| | | KARGQLRTKIESGQGTIPVRSNASIQTWDG | |
| | | VLQGERLLTMSCSDKIARWNVVGIQGSLLS | |
| | | IFVEPIYFSSIILGSLYHGDHLSRAMYQRI | |
| | | SNIEDLPPLYTLNKPLLSGISNAEARQPGK | |
| | | APNFSVNWTVGDSAIEVINATTGKDELGRA | |
| | | SRLCKHALYCRWMRVHGKVPSHLLRSKITK | |
| | | PNVYHESKLAAKEYQAAKARLFTAFIKAGL | |
| | | GAWVEKPTEQDQFSLTGSGSGGVIPDYFKQ | |
| | | SFPEGYSWERSMTYEDGGICIATNDITMEG | |
| | | DSFINKIHFKGTNFPPNGPVMQKRTVGWEA | |
| | | STEKMYERDGVLKGDVKMKLLLKGGGHYRC | |
| | | DYRTTYKVKQKPVKLPDYHFVDHRIEILSH | |
| | | DKDYNKVKLYEHAVARNSTDSMDELYKGGS | |
| | | GGMVSKGEETITSVIKPDMKNKLRMEGNVN | |
| | | GHAFVIEGEGSGKPFEGIQTIDLEVKEGAP | |
| | | LPFAYDILTTAFHYGNRVFTKYPRSGSGSS | |
| | | NRELVVDFLSYKLSQKGYSWSQFSDVEENR | |
| | | TEAPEGTESEMETPSAINGNPSWHLADSPA | |
| | | VNGATGHSSSLDAREVIPMAAVKQALREAG | |
| | | DEFELRYRRAFSDLTSQLHITPGTAYQSFE | |
| | | QVVNELFRDGVNWGRIVAFFSFGGALCVES | |
| | | VDKEMQVLVSRIAAWMATYLNDHLEPWIQE | |
| | | NGGWDTFVELYGNN | |
| MCP- | 163 | MASNFTQFVLVDNGGTGDVTVAPSNFANGI | 88 |
| linker- | | AEWISSNSRSQAYKVTCSVRQSSAQNRKYT | |
| ADAR2- | | IKVEVPKGAWRSYLNMELTIPIFATNSDCE | |
| DDN- | | LIVKAMQGLLKDGNPIPSAIAANSGIYGGS | |
| Bad(L)- | | GSGAGSGSPAGGGAPGSGGGSQLHLPQVLA | |
| ADAR2(E | | DAVSRLVLGKFGDLTDNFSSPHARRKVLAG | |
| 488Q)- | | VVMTTGTDVKDAKVISVSTGTKCINGEYMS | |
| DDC | | DRGLALNDCHAEIISRRSLLRFLYTQLELY | |
| Also | | LNNKDDQKRSIFQKSERGGFRLKENVQFHL | |
| known as | | YISTSPCGDARIFSPHEPILEEPAASGSGT | |
| “nDD- | | GAPPNLWAAQRYGRELRRMSDELVDRHPNR | |
| BAD- | | KARGQLRTKIESGQGTIPVRSNASIQTWDG | |
| cDD” or | | VLQGERLLTMSCSDKIARWNVVGIQGSLLS | |
| “BAD(L) | | IFVEPIYFSSIILGSLYHGDHLSRAMYQRI | |
| Only” | | SNIEDLPPLYTLNKPLLSGISNAEARQPGK | |
| | | APNFSVNWTVGDSAIEVINATTGKDELGRA | |
| | | SRLCKHALYCRWMRVHGKVPSHLLRSKITK | |
| | | PNVYHESKLAAKEYQAAKARLFTAFIKAGL | |
| | | GAWVEKPTEQDQFSLT | |
| MCP- | 164 | MASNFTQFVLVDNGGTGDVTVAPSNFANGI | 89 |
| linker- | | AEWISSNSRSQAYKVTCSVRQSSAQNRKYT | |
| BclxL - | | IKVEVPKGAWRSYLNMELTIPIFATNSDCE | |
| linker - | | LIVKAMQGLLKDGNPIPSAIAANSGIYGGS | |
| ADAR2- | | GSGAGSGSPAGGGAPGSGGGSQSNRELVVD | |
| DDN- | | FLSYKLSQKGYSWSQFSDVEENRTEAPEGT | |
| Bad(L)- | | ESEMETPSAINGNPSWHLADSPAVNGATGH | |
| ADAR2(E | | SSSLDAREVIPMAAVKQALREAGDEFELRY | |
| 488Q)- | | RRAFSDLTSQLHITPGTAYQSFEQVVNELF | |
| DDC - | | RDGVNWGRIVAFFSFGGALCVESVDKEMQV | |
| Also | | LVSRIAAWMATYLNDHLEPWIQENGGWDTF | |
| known as | | VELYGNNAAGGSGGSGGSGGSAAAQLHLPQ | |
| “BclxL- | | VLADAVSRLVLGKFGDLTDNFSSPHARRKV | |
| nDD-BAD- | | LAGVVMTTGTDVKDAKVISVSTGTKCINGE | |
| cDD” | | YMSDRGLALNDCHAEIISRRSLLRFLYTQL | |
| | | ELYLNNKDDQKRSIFQKSERGGFRLKENVQ | |
| | | FHLYISTSPCGDARIFSPHEPILEEPAASG | |
| | | SGTGAPPNLWAAQRYGRELRRMSDELVDRH | |
| | | PNRKARGQLRTKIESGQGTIPVRS | |
| | | NASIQTWDGVLQGERLLTMSCSDKIARWNV | |
| | | VGIQGSLLSIFVEPIYFSSIILGSLYHGDH | |
| | | LSRAMYQRISNIEDLPPLYTLNKPLLSGIS | |
| | | NAEARQPGKAPNFSVNWTVGDSAIEVINAT | |
| | | TGKDELGRASRLCKHALYCRWMRVHGKVPS | |
| | | HLLRSKITKPNVYHESKLAAKEYQAAKARL | |
| | | FTAFIKAGLGAWVEKPTEQDQFSLT | |
| nDD-BAD- | 165 | MASNFTQFVLVDNGGTGDVTVAPSNFANGI | 90 |
| cDD-Bcl- | | AEWISSNSRSQAYKVTCSVRQSSAQNRKYT | |
| XL (MCP- | | IKVEVPKGAWRSYLNMELTIPIFATNSDCE | |
| linker- | | LIVKAMQGLLKDGNPIPSAIAANSGIYGGS | |
| ADAR2- | | GSGAGSGSPAGGGAPGSGGGSQLHLPQVLA | |
| DDN- | | DAVSRLVLGKFGDLTDNFSSPHARRKVLAG | |
| Bad(L)- | | VVMTTGTDVKDAKVISVSTGTKCINGEYMS | |
| ADAR2(E | | DRGLALNDCHAEIISRRSLLRFLYTQLELY | |
| 488Q)- | | LNNKDDQKRSIFQKSERGGFRLKENVQFHL | |
| DDC-Bcl- | | YISTSPCGDARIFSPHEPILEEPAASGSGT | |
| xL | | GAPPNLWAAQRYGRELRRMSDELVDRHPNR | |
| (or Bad(L) | | KARGQLRTKIESGQGTIPVRSNAS | |
| | | IQTWDGVLQGERLLTMSCSDKIARWNVVGI | |
| | | QGSLLSIFVEPIYFSSIILGSLYHGDHLSR | |
| | | AMYQRISNIEDLPPLYTLNKPLLSGISNAE | |
| | | ARQPGKAPNFSVNWTVGDSAIEVINATTGK | |
| | | DELGRASRLCKHALYCRWMRVHGKVPSHLL | |
| | | RSKITKPNVYHESKLAAKEYQAAKARLFTA | |
| | | FIKAGLGAWVEKPTEQDQFSLTGSAAGGSG | |
| | | GSAAASSNRELVVDFLSYKLSQKGYSWSQF | |
| | | SDVEENRTEAPEGTESEMETPSAINGNPSW | |
| | | HLADSPAVNGATGHSSSLDAREVIPMAAVK | |
| | | QALREAGDEFELRYRRAFSDLTSQLHITPG | |
| | | TAYQSFEQVVNELFRDGVNWGRIVAFFSFG | |
| | | GALCVESVDKEMQVLVSRIAAWMATYLNDH | |
| | | LEPWIQENGGWDTFVELYGNNG | |
| MCP- | 166 | MASNFTQFVLVDNGGTGDVTVAPSNFANGI | 91 |
| linker- | | AEWISSNSRSQAYKVTCSVRQSSAQNRKYT | |
| BAD- | | IKVEVPKGAWRSYLNMELTIPIFATNSDCE | |
| ADAR2- | | LIVKAMQGLLKDGNPIPSAIAANSGIYGGS | |
| DD(E488Q | | GSGAGSGSPAGGGAPGSGGGSTGAPPNLWA | |
| ) | | AQRYGRELRRMSDEFVDSFKKASQLHLPQV | |
| Also | | LADAVSRLVLGKFGDLTDNFSSPHARRKVL | |
| known as | | AGVVMTTGTDVKDAKVISVSTGTKCINGEY | |
| “BAD- | | MSDRGLALNDCHAEIISRRSLLRFLYTQLE | |
| DD” | | LYLNNKDDQKRSIFQKSERGGFRLKENVQF | |
| | | HLYISTSPCGDARIFSPHEPILEEPADRHP | |
| | | NRKARGQLRTKIESGQGTIPVRSN | |
| | | ASIQTWDGVLQGERLLTMSCSDKIARWNVV | |
| | | GIQGSLLSIFVEPIYFSSIILGSLYHGDHL | |
| | | SRAMYQRISNIEDLPPLYTLNKPLLSGISN | |
| | | AEARQPGKAPNFSVNWTVGDSAIEVINATT | |
| | | GKDELGRASRLCKHALYCRWMRVHGKVPSH | |
| | | LLRSKITKPNVYHESKLAAKEYQAAKARLF | |
| | | TAFIKAGLGAWVEKPTEQDQFSLT | |
| MCP- | 167 | MASNFTQFVLVDNGGTGDVTVAPSNFANGI | 92 |
| linker- | | AEWISSNSRSQAYKVTCSVRQSSAQNRKYT | |
| BAD- | | IKVEVPKGAWRSYLNMELTIPIFATNSDCE | |
| ADAR2- | | LIVKAMQGLLKDGNPIPSAIAANSGIYGGS | |
| DD(E488Q | | GSGAGSGSPAGGGAPGSGGGSTGAPPNLWA | |
| )-Bcl-xL | | AQRYGRELRRMSDEFVDSFKKASQLHLPQV | |
| Also | | LADAVSRLVLGKFGDLTDNFSSPHARRKVL | |
| known as | | AGVVMTTGTDVKDAKVISVSTGTKCINGEY | |
| “BAD-DD- | | MSDRGLALNDCHAEIISRRSLLRFLYTQLE | |
| BclxL” and | | LYLNNKDDQKRSIFQKSERGGFRLKENVQF | |
| “WT” | | HLYISTSPCGDARIFSPHEPILEEPADRHP | |
| | | NRKARGQLRTKIESGQGTIPVRSN | |
| | | ASIQTWDGVLQGERLLTMSCSDKIARWNVV | |
| | | GIQGSLLSIFVEPIYFSSIILGSLYHGDHL | |
| | | SRAMYQRISNIEDLPPLYTLNKPLLSGISN | |
| | | AEARQPGKAPNFSVNWTVGDSAIEVINATT | |
| | | GKDELGRASRLCKHALYCRWMRVHGKVPSH | |
| | | LLRSKITKPNVYHESKLAAKEYQAAKARLF | |
| | | TAFIKAGLGAWVEKPTEQDQFSLTGSAAAS | |
| | | SNRELVVDFLSYKLSQKGYSWSQFSDVEEN | |
| | | RTEAPEGTESEMETPSAINGNPSWHLADSP | |
| | | AVNGATGHSSSLDAREVIPMAAVKQALREA | |
| | | GDEFELRYRRAFSDLTSQLHITPGTAYQSF | |
| | | EQVVNELFRDGVNWGRIVAFFSFGGALCVE | |
| | | SVDKEMQVLVSRIAAWMATYLNDHLEPWIQ | |
| | | ENGGWDTFVELYGNN | |
| tdMCP AD | 279 | MASNFTQFVLVDNGGTGDVTVAPSNFANGI | 168 |
| AR2-DDN- | | AEWISSNSRSQAYKVTCSVRQSSAQNRKYT | |
| CP5-46- | | IKVEVPKGAWRSYLNMELTIPIFATNSDCE | |
| 4D5E AD | | LIVKAMQGLLKDGNPIPSAIAANSGIYANF | |
| AR2- | | TQFVLVDNGGTGDVTVAPSNFANGIAEWIS | |
| DDC(E488 | | SNSRSQAYKVTCSVRQSSAQNRKYTIKVEV | |
| Q)_(AD- | | PKGAWRSYLNMELTIPIFATNSDCELIVKA | |
| Pep-AD) | | MQGLLKDGNPIPSAIAANSGIYGGSGSGAG | |
| | | SGSPAGGGAPGSGGGSQLHLPQVLADAVSR | |
| | | LVLGKFGDLTDNFSSPHARRKVLAGVVMTT | |
| | | GTDVKDAKVISVSTGTKCINGEYMSDRGLA | |
| | | LNDCHAEIISRRSLLRFLYTQLELYLNNKD | |
| | | DQKRSIFQKSERGGFRLKENVQFHLYISTS | |
| | | PCGDARIFSPHEPILEEPASSGGELDELVY | |
| | | LLDGPGYDPIHCDVVTRGGSHLFNFDRHPN | |
| | | RKARGQLRTKIESGQGTIPVRSNASIQTWD | |
| | | GVLQGERLLTMSCSDKIARWNVVGIQGSLL | |
| | | SIFVEPIYFSSIILGSLYHGDHLSRAMYQR | |
| | | ISNIEDLPPLYTLNKPLLSGISNAEARQPG | |
| | | KAPNFSVNWTVGDSAIEVINATTGKDELGR | |
| | | ASRLCKHALYCRWMRVHGKVPSHLLRSKIT | |
| | | KPNVYHESKLAAKEYQAAKARLFTAFIKAG | |
| | | LGAWVEKPTEQDQFSLT | |
| tdMCP AD | 280 | MASNFTQFVLVDNGGTGDVTVAPSNFANGI | 169 |
| AR2-DDN- | | AEWISSNSRSQAYKVTCSVRQSSAQNRKYT | |
| CP5-46- | | IKVEVPKGAWRSYLNMELTIPIFATNSDCE | |
| 4D5E | | LIVKAMQGLLKDGNPIPSAIAANSGIYANF | |
| ADAR2- | | TQFVLVDNGGTGDVTVAPSNFANGIAEWIS | |
| DDC(E488 | | SNSRSQAYKVTCSVRQSSAQNRKYTIKVEV | |
| Q)_NS4A/ | | PKGAWRSYLNMELTIPIFATNSDCELIVKA | |
| NS3 | | MQGLLKDGNPIPSAIAANSGIYGGSGSGAG | |
| (Genotype 1B) | | SGSPAGGGAPGSGGGSQLHLPQVLADAVSR | |
| | | LVLGKFGDLTDNFSSPHARRKVLAGVVMTT | |
| | | GTDVKDAKVISVSTGTKCINGEYMSDRGLA | |
| | | LNDCHAEIISRRSLLRFLYTQLELYLNNKD | |
| | | DQKRSIFQKSERGGFRLKENVQFHLYISTS | |
| | | PCGDARIFSPHEPILEEPASSGGELDELVY | |
| | | LLDGPGYDPIHCDVVTRGGSHLFNFDRHPN | |
| | | RKARGQLRTKIESGQGTIPVRSNASIQTWD | |
| | | GVLQGERLLTMSCSDKIARWNVVGIQGSLL | |
| | | SIFVEPIYFSSIILGSLYHGDHLSRAMYQR | |
| | | ISNIEDLPPLYTLNKPLLSGISNAEARQPG | |
| | | KAPNFSVNWTVGDSAIEVINATTGKDELGR | |
| | | ASRLCKHALYCRWMRVHGKVPSHLLRSKIT | |
| | | KPNVYHESKLAAKEYQAAKARLFTAFIKAG | |
| | | LGAWVEKPTEQDQFSLTGSAAGGSGGSAAA | |
| | | QGSVVIVGRIILSGSGSITAYSQQTRGLLG | |
| | | CIITSLTGRDKNQVEGEVQVVSTATQSFLA | |
| | | TCVNGVCWTVYHGAGSKTLAGPKGPITQMY | |
| | | TNVDQDLVGWQAPPGARSLTPCTCGSSDLY | |
| | | LVTRHADVIPVRRRGDSRGSLLSPRPVSYL | |
| | | KGSSGGPLLCPSGHAVGIFRAAVCTRGVAK | |
| | | AVDFVPVESMETTMRSESMASNFTQFVLVD | |
| | | NGGTGDVTVAPSNFANGIAEWISSNSRSQA | |
| | | YKVTCSVRQSSAQNRKYTIKVEVPKGAWRS | |
| | | YLNMELTIPIFATNSDCELIVKAMQGLLKD | |
| | | GNPIPSAIAANSGIYGGSGSGAGSGSPAGG | |
| | | GAPGSGGGSQSNRELVVDFLSYKLSQKGYS | |
| | | WSQFSDVEENRTEAPEGTESEMETPSAING | |
| MCP- | 28 | NPSWHLADSPAVNGATGHSSSLDAREVIPM | 198 |
| linker- | | AAVKQALREAGDEFELRYRRAFSDLTSQLH | |
| BclxL - | | ITPGTAYQSFEQVVNELFRDGVNWGRIVAF | |
| linker - | | FSFGGALCVESVDKEMQVLVSRIAAWMATY | |
| ADAR2- | | LNDHLEPWIQENGGWDTFVELYGNNAAGGS | |
| DDN- | | GGSGGSGGSAAAQLHLPQVLADAVSRLVLG | |
| Bad(L)- | | KFGDLTDNFSSPHARRKVLAGVVMTTGTDV | |
| ADAR2(E | | KDAKVISVSTGTKCINGEYMSDRGLALNDC | |
| 488Q)- | | HAEIISRRSLLRFLYTQLELYLNNKDDQKR | |
| DDC; Also | | SIFQKSERGGFRLKENVQFHLYISTSPCGD | |
| known as | | ARIFSPHEPILEEPAASGSGTGAPPNLWAA | |
| “BclxL- | | QRYGRELRRMSDELVDRHPNRKARGQLRTK | |
| nDD-BAD- | | IESGQGTIPVRSNASIQTWDGVLQ | |
| CDD” | | GERLLT | |
| | | MSCSDKIARWNVVGIQGSLLSIFVEPIYFS | |
| | | SIILGSLYHGDHLSRAMYQRISNIEDLPPL | |
| | | YTLNKPLLSGISNAEARQPGKAPNFSVNWT | |
| | | VGDSAIEVINATTGKDELGRASRLCKHALY | |
| | | CRWMRVHGKVPSHLLRSKITKPNVYHESKL | |
| | | AAKEYQAAKARLFTAFIKAGLGAWVEKPTE | |
| | | QDQFSLT | |
| MCP- | 282 | ASNFTQFVLVDNGGTGDVTVAPSNFANGIA | 200 |
| linker- | | EWISSNSRSQAYKVTCSVRQSSAQNRKYTI | |
| BAD- | | KVEVPKGAWRSYLNMELTIPIFATNSDCEL | |
| ADAR2- | | IVKAMQGLLKDGNPIPSAIAANSGIYGGSG | |
| DD | | SGAGSGSPAGGGAPGSGGGSTGAPPNLWAA | |
| (E488Q) | | QRYGRELRRMSDEFVDSFKKASQLHLPQVL | |
| Also | | ADAVSRLVLGKFGDLTDNFSSPHARRKVLA | |
| known as | | GVVMTTGTDVKDAKVISVSTGTKCINGEYM | |
| “BAD- | | SDRGLALNDCHAEIISRRSLLRFLYTQLEL | |
| DD” | | YLNNKDDQKRSIFQKSERGGFRLKENVQFH | |
| | | LYISTSPCGDARIFSPHEPILEEPADRHPN | |
| | | RKARGQLRTKIESGQGTIPVRSNA | |
| | | SIQTWD | |
| | | GVLQGERLLTMSCSDKIARWNVVGIQGSLL | |
| | | SIFVEPIYFSSIILGSLYHGDHLSRAMYQR | |
| | | ISNIEDLPPLYTLNKPLLSGISNAEARQPG | |
| | | KAPNFSVNWTVGDSAIEVINATTGKDELGR | |
| | | ASRLCKHALYCRWMRVHGKVPSHLLRSKIT | |
| | | KPNVYHESKLAAKEYQAAKARLFTAFIKAG | |
| | | LGAWVEKPTEQDQFSLT | |
| MCP- | 283 | MASNFTQFVLVDNGGTGDVTVAPSNFANGI | 202 |
| linker- | | AEWISSNSRSQAYKVTCSVRQSSAQNRKYT | |
| BAD- | | IKVEVPKGAWRSYLNMELTIPIFATNSDCE | |
| ADAR2- | | LIVKAMQGLLKDGNPIPSAIAANSGIYGGS | |
| DD(E488Q | | GSGAGSGSPAGGGAPGSGGGSTGAPPNLWA | |
| )-Bcl-xL | | AQRYGRELRRMSDEFVDSFKKASQLHLPQV | |
| Also | | LADAVSRLVLGKFGDLTDNFSSPHARRKVL | |
| known as | | AGVVMTTGTDVKDAKVISVSTGTKCINGEY | |
| “BAD-DD- | | MSDRGLALNDCHAEIISRRSLLRFLYTQLE | |
| BclxL” and | | LYLNNKDDQKRSIFQKSERGGFRLKENVQF | |
| “WT” | | HLYISTSPCGDARIFSPHEPILEEPADRHP | |
| | | NRKARGQLRTKIESGQGTIPVRSNASIQTW | |
| | | DGVLQGERLLTMSCSDKIARWNVV | |
| | | GIQGSL | |
| | | LSIFVEPIYFSSIILGSLYHGDHLSRAMYQ | |
| | | RISNIEDLPPLYTLNKPLLSGISNAEARQP | |
| | | GKAPNFSVNWTVGDSAIEVINATTGKDELG | |
| | | RASRLCKHALYCRWMRVHGKVPSHLLRSKI | |
| | | TKPNVYHESKLAAKEYQAAKARLFTAFIKA | |
| | | GLGAWVEKPTEQDQFSLTGSAAASSNRELV | |
| | | VDFLSYKLSQKGYSWSQFSDVEENRTEAPE | |
| | | GTESEMETPSAINGNPSWHLADSPAVNGAT | |
| | | GHSSSLDAREVIPMAAVKQALREAGDEFEL | |
| | | RYRRAFSDLTSQLHITPGTAYQSFEQVVNE | |
| | | LFRDGVNWGRIVAFFSFGGALCVESVDKEM | |
| | | QVLVSRIAAWMATYLNDHLEPWIQENGGWD | |
| | | TFVELYGNN | |
| tdMCP_AD | 284 | MASNFTQFVLVDNGGTGDVTVAPSNFANGI | 204 |
| AR2-DDN- | | AEWISSNSRSQAYKVTCSVRQSSAQNRKYT | |
| CP5-46- | | IKVEVPKGAWRSYLNMELTIPIFATNSDCE | |
| 4D5E_AD | | LIVKAMQGLLKDGNPIPSAIAANSGIYANF | |
| AR2- | | TQFVLVDNGGTGDVTVAPSNFANGIAEWIS | |
| DDC(E488 | | SNSRSQAYKVTCSVRQSSAQNRKYTIKVEV | |
| Q) (AD- | | PKGAWRSYLNMELTIPIFATNSDCELIVKA | |
| Pep-AD) | | MQGLLKDGNPIPSAIAANSGIYGGSGSGAG | |
| | | SGSPAGGGAPGSGGGSQLHLPQVLADAVSR | |
| | | LVLGKFGDLTDNFSSPHARRKVLAGVVMTT | |
| | | GTDVKDAKVISVSTGTKCINGEYMSDRGLA | |
| | | LNDCHAEIISRRSLLRFLYTQLELYLNNKD | |
| | | DQKRSIFQKSERGGFRLKENVQFHLYISTS | |
| | | PCGDARIFSPHEPILEEPASSGGELDELVY | |
| | | LLDGPGYDPIHCDVVTRGGSHLFNFDRHPN | |
| | | RKARGQLRTKIESGQGTIPVRSNASIQTWD | |
| | | GVLQGERLLTMSCSDKIARWNVVGIQGSLL | |
| | | SIFVEPIYFSSIILGSLYHGDHLSRAMYQR | |
| | | ISNIEDLPPLYTLNKPLLSGISNAEARQPG | |
| | | KAPNFSVNWTVGDSAIEVINATTGKDELGR | |
| | | ASRLCKHALYCRWMRVHGKVPSHLLRSKIT | |
| | | KPNVYHESKLAAKEYQAAKARLFTAFIKAG | |
| | | LGAWVEKPTEQDQFSLT | |
| tdMCP_AD | 285 | MASNFTQFVLVDNGGTGDVTVAPSNFANGI | 206 |
| AR2-DDN- | | AEWISSNSRSQAYKVTCSVRQSSAQNRKYT | |
| CP5-46- | | IKVEVPKGAWRSYLNMELTIPIFATNSDCE | |
| 4D5E | | LIVKAMQGLLKDGNPIPSAIAANSGIYANF | |
| ADAR2- | | TQFVLVDNGGTGDVTVAPSNFANGIAEWIS | |
| DDC(E488 | | SNSRSQAYKVTCSVRQSSAQNRKYTIKVEV | |
| Q)_NS4A/ | | PKGAWRSYLNMELTIPIFATNSDCELIVKA | |
| NS3 | | MQGLLKDGNPIPSAIAANSGIYGGSGSGAG | |
| (Genotype 1B) | | SGSPAGGGAPGSGGGSQLHLPQVLADAVSR | |
| | | LVLGKFGDLTDNFSSPHARRKVLAGVVMTT | |
| | | GTDVKDAKVISVSTGTKCINGEYMSDRGLA | |
| | | LNDCHAEIISRRSLLRFLYTQLELYLNNKD | |
| | | DQKRSIFQKSERGGFRLKENVQFHLYISTS | |
| | | PCGDARIFSPHEPILEEPASSGGELDELVY | |
| | | LLDGPGYDPIHCDVVTRGGSHLFNFDRHPN | |
| | | RKARGQLRTKIESGQGTIPVRSNASIQTWD | |
| | | GVLQGERLLTMSCSDKIARWNVVGIQGSLL | |
| | | SIFVEPIYFSSIILGSLYHGDHLSRAMYQR | |
| | | ISNIEDLPPLYTLNKPLLSGISNAEARQPG | |
| | | KAPNFSVNWTVGDSAIEVINATTGKDELGR | |
| | | ASRLCKHALYCRWMRVHGKVPSHLLRSKIT | |
| | | KPNVYHESKLAAKEYQAAKARLFTAFIKAG | |
| | | LGAWVEKPTEQDQFSLTGSAAGGSGGSAAA | |
| | | QGSVVIVGRIILSGSGSITAYSQQTRGLLG | |
| | | CIITSLTGRDKNQVEGEVQVVSTATQSFLA | |
| | | TCVNGVCWTVYHGAGSKTLAGPKGPITQMY | |
| | | TNVDQDLVGWQAPPGARSLTPCTCGSSDLY | |
| | | LVTRHADVIPVRRRGDSRGSLLSPRPVSYL | |
| | | KGSSGGPLLCPSGHAVGIFRAAVCTRGVAK | |
| | | AVDFVPVESMETTMRSES | |

In some embodiments, the methods, compositions and systems disclosed herein relate to an iAD which is an iADAR2. Exemplary iADAR2 are disclosed in Table 5.

In some embodiments of the aspects, an iADAR2 fusion protein for use in the methods and compositions as disclosed herein is selected from any of SEQ ID NO: 150-167, SEQ ID NO: 279-285 or a sequence that has at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% sequence identity to an amino acid sequence selected from any of SEQ ID NO: 150-167 or SEQ ID NO: 279-285, and that maintains the same functions as the sequence from which is it derived.

In some embodiments, an exemplary iADAR2 has the ADAR2-DD in one polypeptide, and is selected from any of SEQ ID NO: 166 or 167, or a polypeptide that has at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% sequence identity to an amino acid sequence selected from SEQ ID NO: 166 or 167, and that maintains the same functions as the sequence from which is it derived.

In some embodiments, an exemplary iADAR2 has the ADAR2-DD in one polypeptide in combination with NS3 and its cognate peptide domain (e.g., CP5-46-4D5E). Such an iADAR2 can comprise one of SEQ ID NO: 169, 206, 280, or 285, or a polypeptide that has at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% sequence identity to one of SEQ ID NO: 169, 206, 280, or 285, and that maintains the same functions as the sequence from which is it derived.

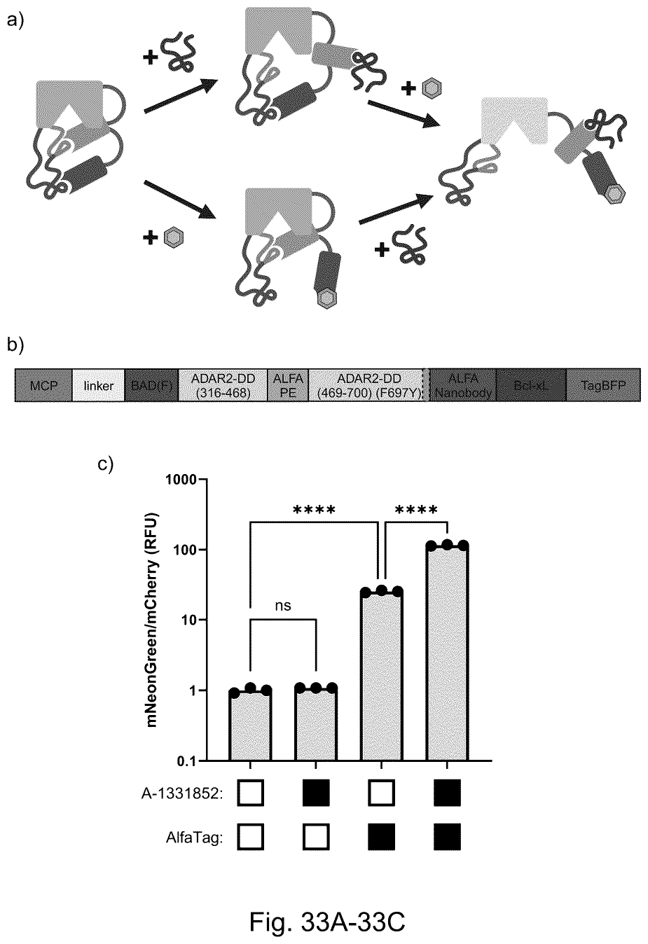

In one aspect, described herein is a fusion protein comprising: (a) a first portion of a deaminase domain (DD) of an adenosine deaminase; (b) a first member of a first binding pair associated with the first portion of the DD; (c) a second portion of the DD; (d) a second member of a first binding pair associated with the second portion of the DD; (e) a first member of a second binding pair associated with the first member of the first binding pair; and (f) a second member of the second binding pair associated with the second member of the first binding pair (see e.g., FIG. 33A-33C). In some embodiments, the first member of the first binding pair is capable of binding to the second member of the first binding pair in the absence of a first inducer, resulting in allosteric inhibition of the first and second portions of the DD. In some embodiments, the first member of the first binding pair is not capable of binding to the second member of the first binding pair in the presence of the first inducer, resulting in activation of the first and second portions of the DD. In some embodiments, the first member of the second binding pair is capable of binding to the second member of the second binding pair in the absence of a second inducer, resulting in allosteric inhibition of the first and second portions of the DD. In some embodiments, the first member of the second binding pair is not capable of binding to the second member of the second binding pair in the presence of the second inducer, resulting in activation of the first and second portions of the DD. In some embodiments, the fusion protein further comprises a third, fourth, fifth, sixth, seventh, eighth, ninth, tenth or more binding pairs.

In some embodiments, an iAD polypeptide can comprise one of SEQ ID NO: 334-339 or 342-355 or a polypeptide that has at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% sequence identity to one of SEQ ID NO: SEQ ID NO: 334-339 or 342-355, and that maintains the same functions as the sequence from which is it derived.

In some embodiments, an AD polypeptide can be encoded by a nucleic acid comprising one of SEQ ID NO: 390-392 or a nucleic acid that has at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% sequence identity to one of SEQ ID NO: 390-392, and that maintains the same functions when expressed as a polypeptide as the sequence from which is it derived.

### II. Synthetic Effector Constructs

As disclosed herein in the Examples, the iADAR was demonstrated to edit stop codons in a synthetic mRNA transcript, where the synthetic construct comprises the STOP codon located in a small hair-pin, referred to herein as “ds-STOP” region. In particular, as disclosed herein the iADAR2 is specifically modified and engineered so it could edit a STOP by using a short hairpin motif, thereby enabling the stop codon editing on the same nucleic acid strand that GOI is expressed, and that is bound to by the DD. In some embodiments, a ds-TC region (e.g., ds-STOP, ds-START, or ds-SENSE region) is in a synthetic construct as disclosed herein, e.g., a TIC or TAC as disclosed herein.

Another aspect of the technology relates to synthetic nucleic acid constructs the iADAR effectuates. In particular, another aspect of the technology described herein relates to synthetic nucleic acid constructs that function as a target activation construct (TAC) or target inactivation construct (TIC), where the synthetic TAC comprises a hairpin loop comprising a STOP codon located upstream (i.e., 5′) of a nucleic acid encoding Gene of Interest (GOI), and where a synthetic TIC comprises a hairpin loop comprising a STOP codon located downstream (i.e., 3′) of a GOI and upstream (i.e., 5′) of a nucleic acid poly A sequence. Another aspect relates to nucleic acid encoding an iADAR and/or a nucleic acid encoding one or more of an activation construct (TAC) or inactivation construct (TIC).

In some embodiments, synthetic constructs, referred to herein as Target Activation Constructs (TAC) or Target inactivation constructs (TIC) are exemplary mRNA transcripts that can be edited by iADAR when it is in the active state (iADAR-ON).

It is envisioned that the iADAR can edit any synthetic construct comprising a hairpin loop with a STOP codon located within the hairpin loop, which are referred to herein as “ds-STOP” regions. Such synthetic constructs comprising a ds-TC region (e.g., ds-STOP, ds-START, or ds-SENSE region) as defined herein, can be delivered to a cell, e.g., a human cell by any means, including but not limited to using viral vectors and non-viral vectors as described herein, and/or use of CRISPR/Cas systems.

Accordingly, in some embodiments, the ds-TC region (e.g., ds-STOP, ds-START, or ds-SENSE region) can be inserted into a nucleic acid sequence in the genome of a cell using gene editing technologies, including but not limited to CRISPR or other gene-editing technologies. Depending where the ds-STOP is inserted into the nucleic acid sequence, e.g., genome of a cell, if it is inserted upstream of a polyA sequence to a particular transcript, such embodiment could enable iADAR mediated mRNA decay of a particular gene or transcript in a cell, which could be turned on by the presence of an inducer.

In some embodiments, the synthetic effector constructs described herein comprise synthetic RNA. In some embodiments, the synthetic effector constructs described herein comprise synthetic mRNA. In some embodiments, the synthetic effector constructs described herein comprise synthetic circular RNA.

In some embodiments, the synthetic RNA molecule comprises one of SEQ ID NOs: 292-327 or 356-357 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 292-327 or 356-357, that maintains the same function.

### A. Double-Stranded Region of the RNA

Described herein are RNA molecules comprising at least one double-stranded region. In some embodiments, the double-stranded region of the RNA comprises (i) at least one target codon; and (ii) an RNA binding motif capable of being bound by an RNA-binding domain (e.g., of an iAD polypeptide as described further herein). In some embodiments, the double-stranded region comprises secondary structure of the RNA. In some embodiments, the double-stranded region comprises at least one hairpin. In some embodiments, the double-stranded region comprises at least two hairpins.

In some embodiments, the double-stranded region is at least 10 nucleotides long, at least 20 nucleotides long, at least 30 nucleotides long, at least 40 nucleotides long, at least 50 nucleotides long, at least 60 nucleotides long, at least 70 nucleotides long, at least 80 nucleotides long, at least 90 nucleotides long, at least 100 nucleotides long, or more. In some embodiments, the double-stranded region is at most 10 nucleotides long, at most 20 nucleotides long, at most 30 nucleotides long, at most 40 nucleotides long, at most 50 nucleotides long, at most 60 nucleotides long, at most 70 nucleotides long, at most 80 nucleotides long, at most 90 nucleotides long, or at most 100 nucleotides long. In some embodiments, the double-stranded region is about 10 nucleotides long, about 20 nucleotides long, about 30 nucleotides long, about 40 nucleotides long, about 50 nucleotides long, about 60 nucleotides long, about 70 nucleotides long, about 80 nucleotides long, about 90 nucleotides long, or about 100 nucleotides long. In some embodiments, the double-stranded region is about 55-65 nucleotides long, about 50-70 nucleotides long, about 45-75 nucleotides long, or about 40-80 nucleotides long.

i. Target Codons

Described herein are RNA molecules comprising at least one target codon. In some embodiments, the RNA molecule comprises 1, 2, 3, 4, 5, 6, 7, 8, 9, 10, or more target codons. In some embodiments, the at least one target codon is present in a double-stranded region of the RNA molecule. In some embodiments, the target codon is a double-stranded target codon (dsTC). In some embodiments, the at least one target codon is in close proximity to at least one RNA binding motif capable of being bound by an RNA-binding domain (e.g., of an iAD polypeptide as described further herein). In some embodiments, the target codon is a stop codon (e.g., a double-stranded stop codon, ds-STOP). In some embodiments, the target codon is a start codon (e.g., a double-stranded start codon, ds-START). In some embodiments, the target codon is a non-start codon (e.g., a double-stranded non-start codon, ds-non-START). In some embodiments, the target codon is a sense codon (e.g., a double-stranded sense codon, ds-SENSE).

In some embodiments, the target codon is upstream of at least one open reading frame. In some embodiments, the target codon is downstream of at least one open reading frame. In some embodiments, the target codon is within at least one open reading frame. In some embodiments, action of the induced iAD on the target codon results in activation, deactivation, or alteration to translation of an associated open reading, depending on the structure of the RNA molecule. Table 16 contains non-limiting examples of target codons in RNA molecules and their effect on RNA structure and/or function.

TABLE 16
Exemplary RNA molecule types

| double-stranded | | Type of RNA |
|---|---|---|
| target codon | Location | construct (activity |
| (ds-TC) change by | of ds-TC in | in presence of |
| induced iAD | RNA molecule | induced iAD) |
| STOP → non-STOP | 5′ of ORF or | Activation construct |
| | middle of ORF | (OFF → ON) |
| STOP → non-STOP | 3′ of ORF | Inactivation construct |
| | | (ON → OFF) |
| START → non-START | 5′ of ORF | Inactivation construct |
| | | (ON → OFF) |
| START → non-START | 5′ of ORF or | Altered initiation site → |
| | middle of ORF | protein length variants |
| non-START → START | 5′ of ORF or | Activation construct |
| | middle of ORF | (OFF → ON) |
| sense 1 → sense 2 | Any sense | Altered codon → RNA |
| | codon in ORF | functional variants (e.g., |
| | | splicing, translation, |
| | | degradation, etc.) and/or |
| | | Altered amino acid → |
| | | protein mutation variants |

a. Ds-STOP Regions

As disclosed herein, an iADAR in the ON state can edit a STOP codon in a RNA transcript, where the STOP codon is located a double stranded region in the transcript, and where the iADAR can bind to the double stranded region to eliminate the STOP signal. Accordingly, in some embodiments, the STOP codon is located in double stranded region, herein referred to as “ds-STOP” region. In some embodiments, the ds-STOP region is a short hairpin loop, where the short hairpin loop is RNA or mRNA.

In some embodiments, the ds-STOP region is a double stranded RNA transcript that comprises (i) a STOP codon as disclosed herein, and (ii) a Binding motif for RBD (BM), where the Binding motif for RBD (BM) binds to a RNA-binding domain (RBD) of the iADAR. In some embodiments, the Binding motif for RBD (BM) is capable of being bound by an RNA-binding domain of the DD.

In some embodiments, the ds-STOP region comprises at least one hairpin. In some embodiments, the ds-STOP region comprises at least one hairpin comprising the at least one stop codon and the Binding motif for RBD (BM). In some embodiments, the ds-STOP region comprises a first hairpin comprising the at least one stop codon and a second hairpin comprising the Binding motif for RBD (BM).

As disclosed herein, in the presence of an inducer the iADAR changes an A to an I in mRNA. In some embodiments, the ds-STOP region comprises a stop codon UAG, which is edited to a UIG codon in the presence of an inducer. In some embodiments, the mRNA STOP codon present in the ds-STOP region is selected from any of: UAA, UAG, or UGA. Accordingly, in the presence of an inducer, the iADAR-ON edits the STOP codon UAA to UII, or STOP codon UAG to UIG or STOP codon UGA to UGI, therefore eliminating the STOP codon in each case.

In some embodiments, the ds-STOP region comprises at least one stop codon, where the Stop codon comprises UAG. In some embodiments, the ds-STOP region comprises at least one non-stop codon, for example, where the non-stop codon comprises at least one tryptophan codon, e.g., a tryptophan codon comprises UGG.

In some embodiments, the ds-STOP region comprises a STOP sequence selected from any of SEQ ID NO: 105-110, 170, 174, 178, 182, 186, 190, or 194, or a nucleic acid sequence comprising at least 10 consecutive nucleotides selected from SEQ ID NO: 105-110, 170, 174, 178, 182, 186, 190, or 194, or a nucleic acid sequence having at least 70%, or about 75%, or about 80%, or about 85%, or about 90%, or about 92%, or about 94% or about 96% or about 98%, or about 99% or 100% sequence identity to any of SEQ ID NO: 105-110, 170, 174, 178, 182, 186, 190, or 194.

TABLE 6
Exemplary STOP sequences comprising stop Codons in a ds-STOP region:

| | Stop Codon sequence | |
|---|---|---|
| SEQ ID | in ds-STOP region | |
| 105 | UAG-UAG-MS2 | CGCGTAGCGCTAGCTTTGCCAGCGCCACGCGaaACAT |
| | | GAGGATcACCCATGT |
| 106 | UGG-UGG | CGCGTGGCGCTGGCTTCCTTGCCAGCGCCACGCG |
| 107 | UAG-UGG | CGCGTAGCGCTGGCTTCCTTGCCAGCGCCACGCG |
| 108 | UAG-UAG | CGCGTAGCGCTAGCTTCCTTGCCAGCGCCACGCG |
| 109 | UAG-UAG-PP7 | CGCGTAGCGCTAGCTTTGCCAGCGCCACGCGaaggagca |
| | | gacgatatggcgtcgctcc |
| 110 | UAG-UAG-BoxB | CGCGTAGCGCTAGCTTTGCCAGCGCCACGCGgtaagggc |
| | | cctgaagaagggccc |
| 111 | UAG-UAG-HIV | CGCGTAGCGCTAGCTTTGCCAGCGCCACGCGgtaggctcg |
| | TAR | tctgagctcattagctccgagcc |
| 170 | UAG-UAG Stop | AATTCCGCGTAGCGCTAGCTTTGCCAGCGCCACG |
| | Loop (bolded) w/ | CGaaACATGAGGATcACCCATGTACTAGT |
| | MS2 Loop | |
| | (italicized) | |
| 174 | UAG-UGG Stop | AATTCCGCGTAGCGCTGGCTTTGCCAGCGCCACG |
| | Loop (bolded) w/ | CGaaACATGAGGATcACCCATGTACTAGT |
| | MS2 Loop | |
| | (italicized) | |
| 178 | UGG-UAG Stop | AATTCCGCGTGGCGCTAGCTTTGCCAGCGCCACG |
| | Loop (bolded) w/ | CGaaACATGAGGATcACCCATGTACTAGT |
| | MS2 Loop | |
| | (italicized) | |
| 182 | UAG-UAG Stop | AATTCCGCGTAGCGCTAGCTACATGAGGATcACCCAT |
| | Loop (bolded) | GTTGCCAGCGCCACGCGACTAGT |
| | w/Internal MS2 Loop | |
| | (italicized) | |
| 186 | UAG-UAG Stop | AATTCCGCGTAGCGCTAGCTTTGCCAGCGCCACG |
| | Loop (bolded) w/ | CGaaggagcagacgatatggcgtcgctccaaTACTAGT |
| | PP7 Loop (italicized) | |
| 190 | UAG-UAG Stop | AATTCCGCGTAGCGCTAGCTTTGCCAGCGCCACG |
| | Loop (bolded) w/ | CGGtaggctcgtctgagctcattagctccgagccaACTAGT |
| | HIV Tar Loop | |
| | (italicized) | |
| 194 | UAG-UAG Stop | AATTCCGCGTAGCGCTAGCTTTGCCAGCGCCACG |
| | Loop (bolded) w/ | CGGtaagggccctgaagaagggcccaACTAGT |
| | BoxB Loop | |

In some embodiments, the STOP sequence comprises a P2A-T2A sequence 5′ and/or 3′ of any stop sequence, e.g., a stop sequence selected from any of SEQ ID NO: 105-110, or a sequence having at least 85% sequence identity to any of SEQ ID NO: 105-110. In some embodiments, a P2A-T2A sequence encodes an amino acid comprising the sequence of: ATNFSLLKQAGDVEENPGPASAGSGEGRGSLLTCGDVEENPGP (SEQ ID NO: 112). In some embodiments, the ds-STOP region comprises a sequence selected from SEQ ID NO: 112 or a sequence at least 85% sequence identity to SEQ ID NO: 112.

b. Ds-START or Ds-Non-START Regions

As disclosed herein, an iADAR in the ON state can edit a START codon in a RNA transcript, where the START codon is located a double stranded region in the transcript, and where the iADAR can bind to the double stranded region to eliminate the START signal. Accordingly, in some embodiments, the START codon is located in double stranded region, herein referred to as “ds-START” region. In some embodiments, the ds-START region is a short hairpin loop, where the short hairpin loop is RNA or mRNA.

In some embodiments, the ds-START region is a double stranded RNA transcript that comprises (i) a START codon as disclosed herein, and (ii) a Binding motif for RBD (BM), where the Binding motif for RBD (BM) binds to a RNA-binding domain (RBD) of the iADAR. In some embodiments, the Binding motif for RBD (BM) is capable of being bound by an RNA-binding domain of the DD.

In some embodiments, the ds-START region comprises at least one hairpin. In some embodiments, the ds-START region comprises at least one hairpin comprising the at least one start codon and the Binding motif for RBD (BM). In some embodiments, the ds-START region comprises a first hairpin comprising the at least one START codon and a second hairpin comprising the Binding motif for RBD (BM).

As disclosed herein, in the presence of an inducer the iADAR changes an A to an I in mRNA. In some embodiments, the ds-non-START region comprises a non-start codon AUA, which is edited to a AUI start codon in the presence of an inducer. In some embodiments, the ds-START region comprises a start codon AUG, which is edited to a IUG non-start codon in the presence of an inducer. In some embodiments, the mRNA START codon present in the ds-START region is selected from any of: AUI or AUG. Accordingly, in the presence of an inducer, the iADAR-ON edits the START codon AUG to IUG, therefore eliminating the START codon and deactivating translation. In other embodiments, in the presence of an inducer, the iADAR-ON edits the non-START codon AUA to START codon AUI, therefore adding a START codon and activating translation.

In some embodiments, the ds-START region comprises at least one start codon, where the start codon comprises AUI or AUG. In some embodiments, the ds-non-START region comprises at least one non-start codon, including but not limited to AUA or IUG.

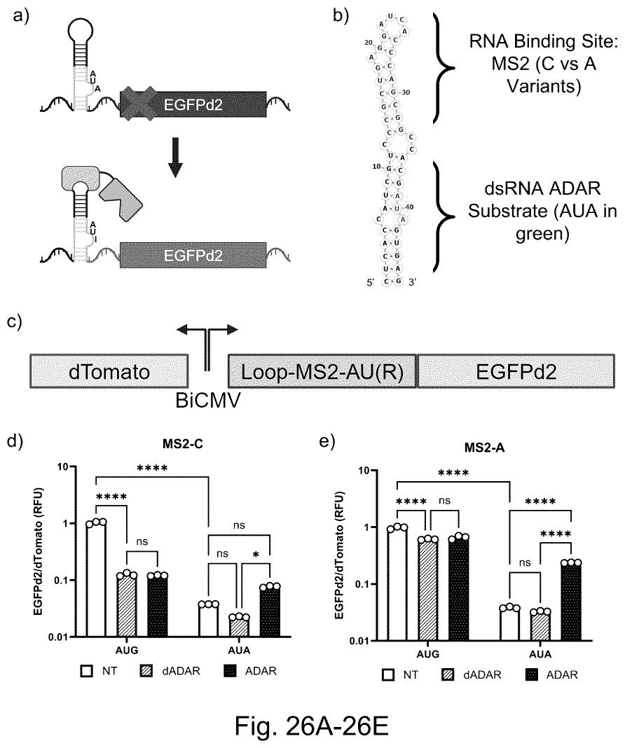

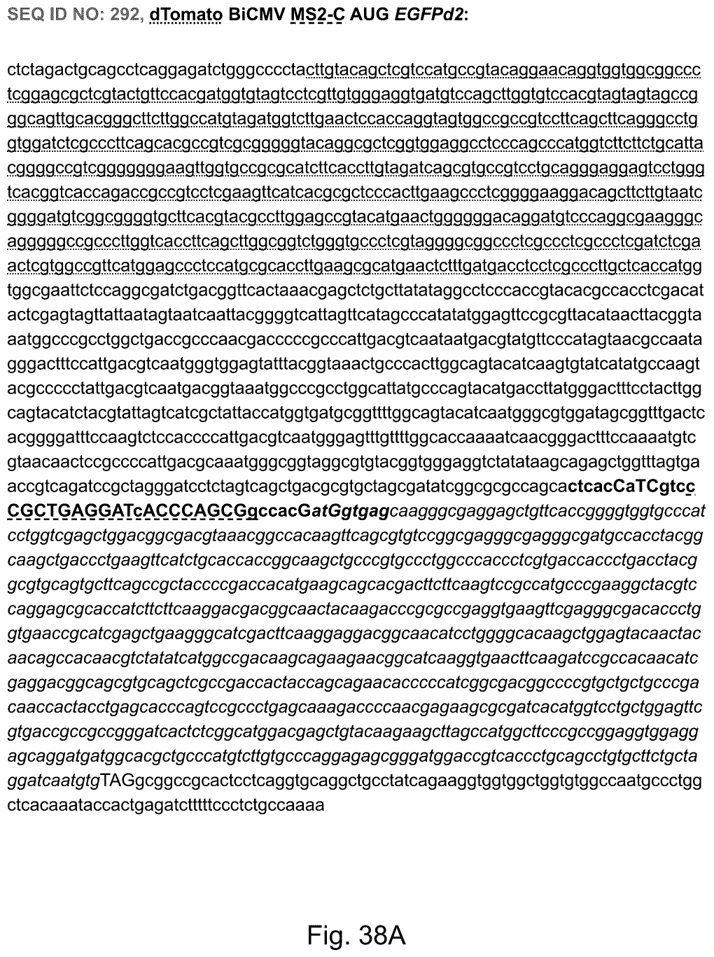

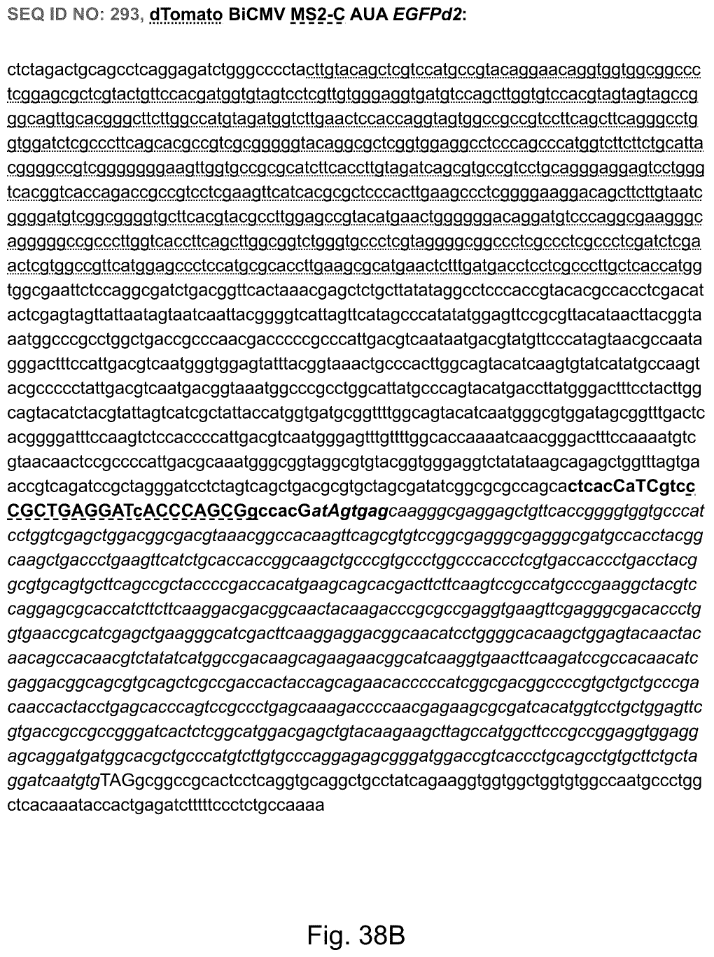

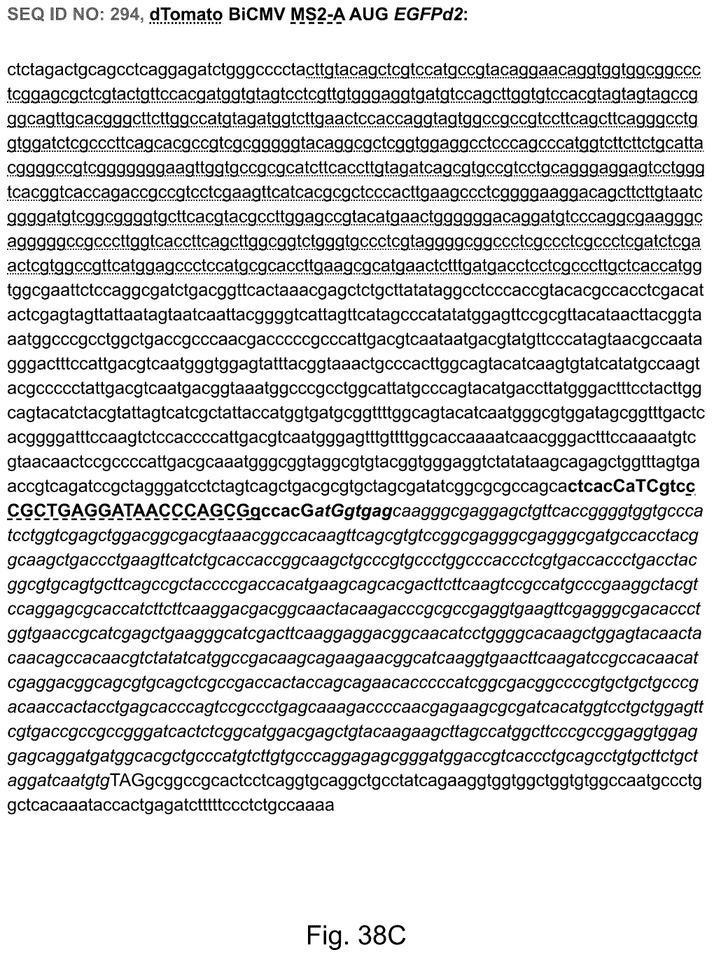

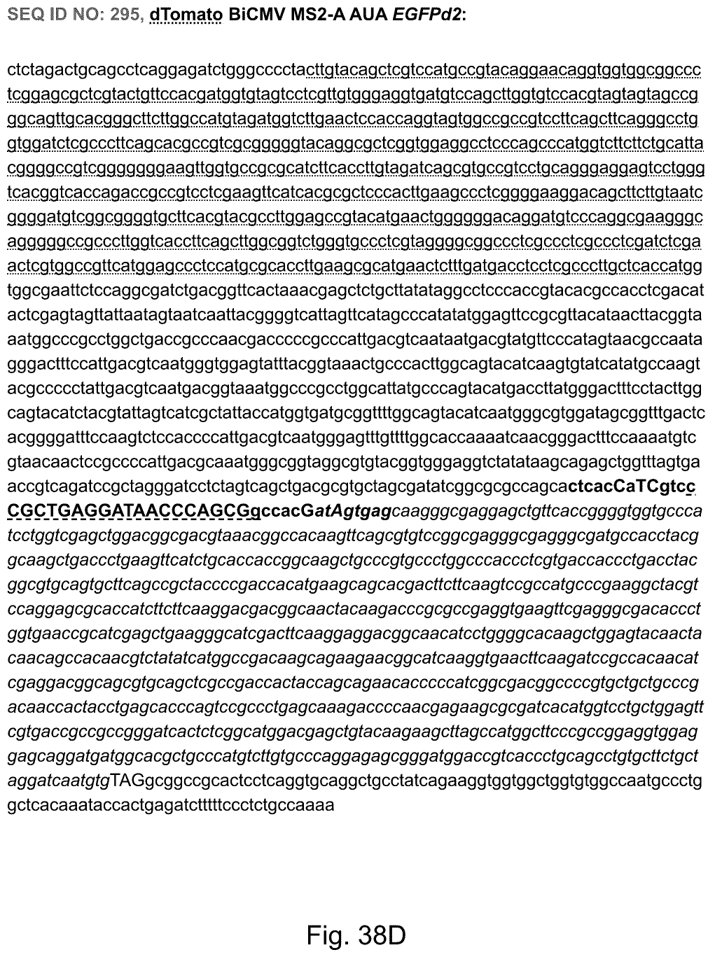

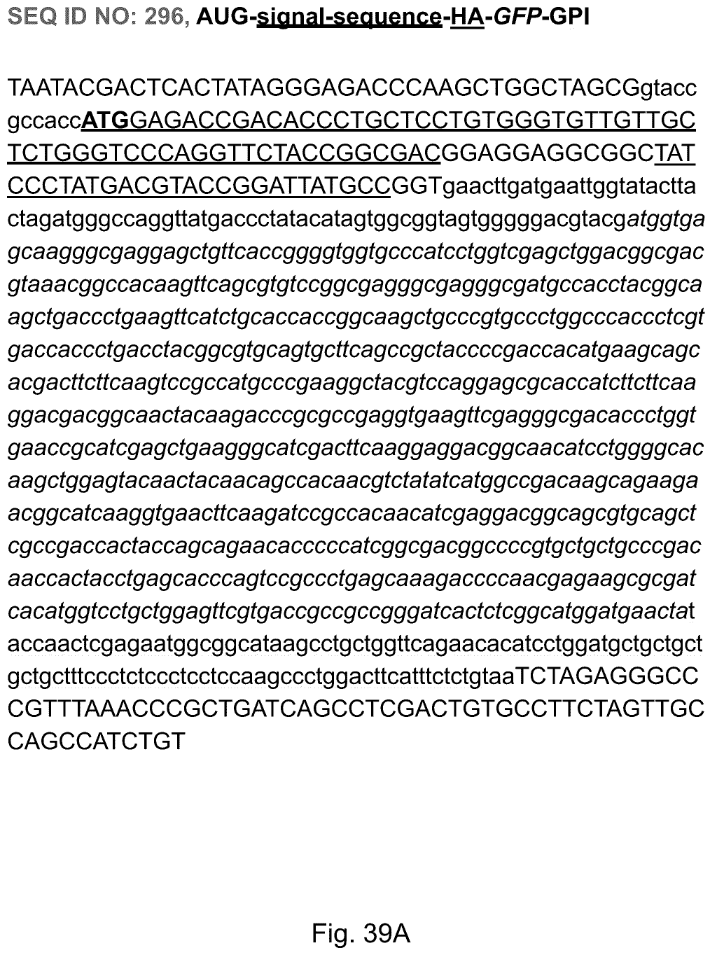

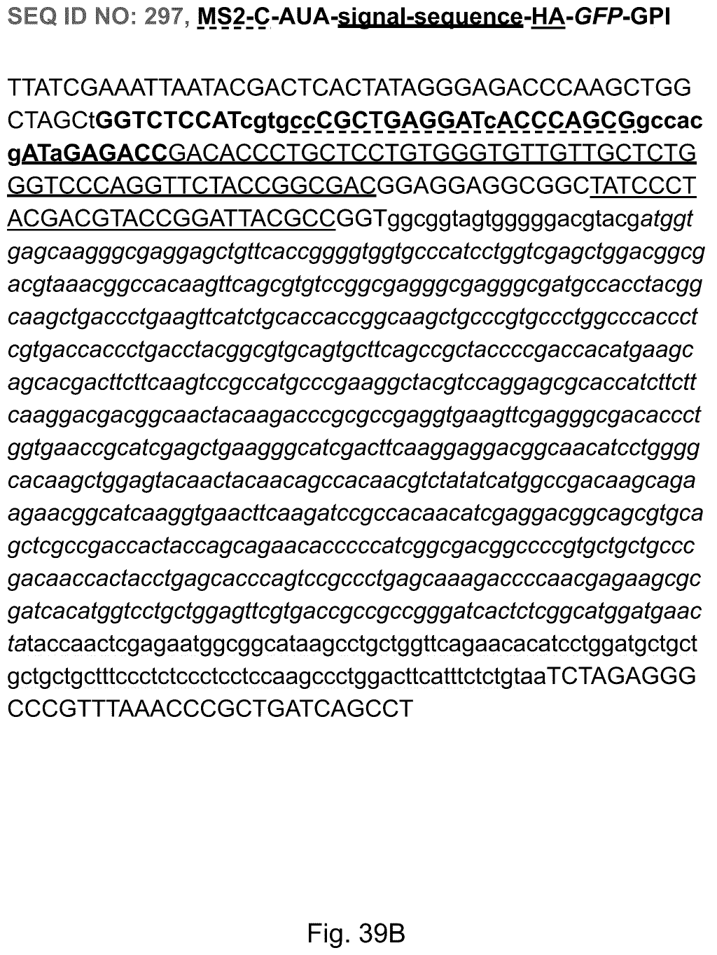

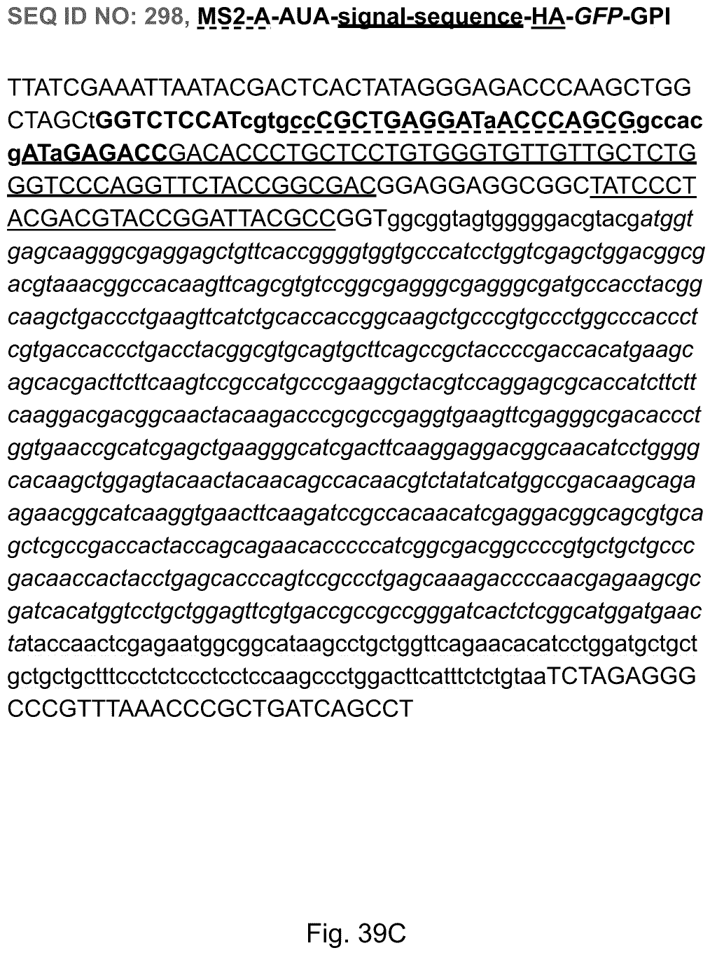

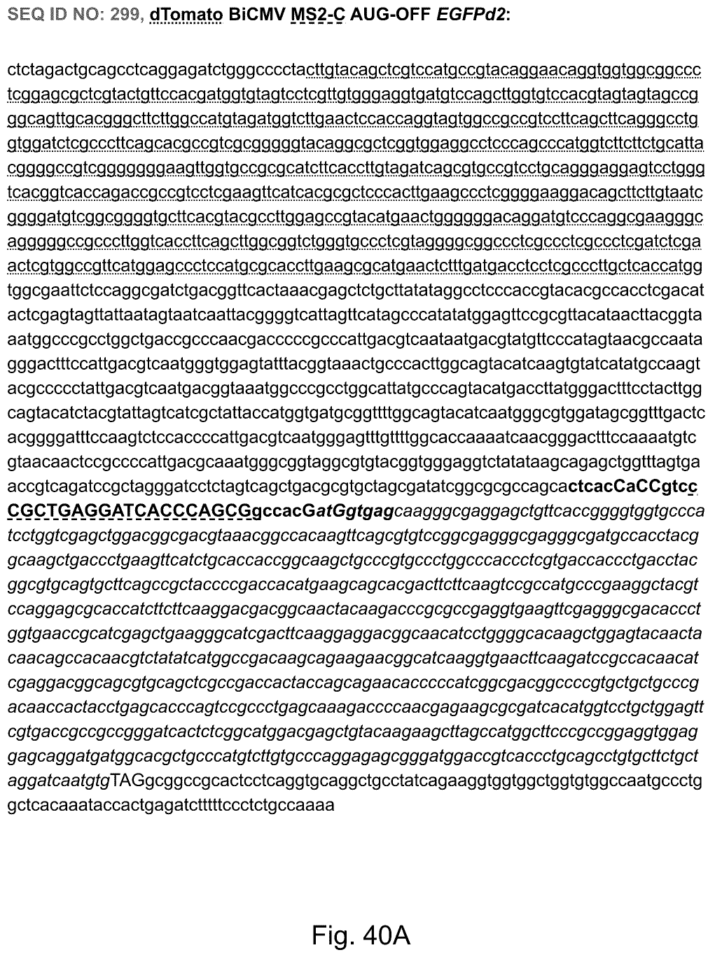

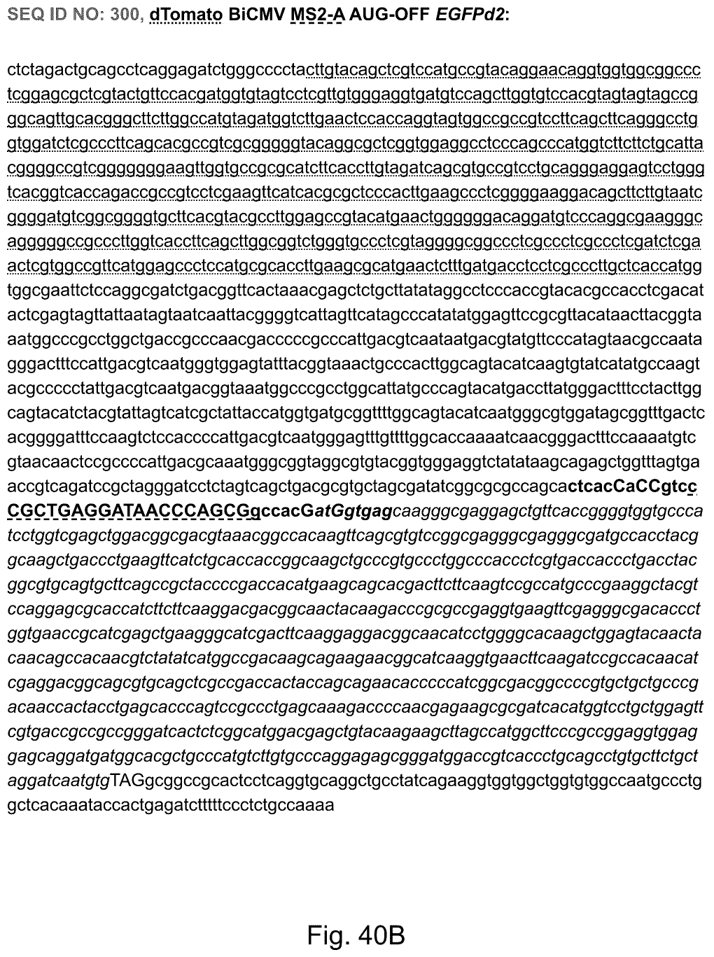

In some embodiments, the ds-START region comprises a start or non-start sequence as tested in any of FIGS. 26-28, as shown in any of FIGS. 38-40, or included in any of SEQ ID NO: 292-300 or a nucleic acid sequence comprising at least 10 consecutive nucleotides selected from SEQ ID NO: 292-300, or a nucleic acid sequence having at least 70%, or about 75%, or about 80%, or about 85%, or about 90%, or about 92%, or about 94% or about 96% or about 98%, or about 99% or 100% sequence identity to any of SEQ ID NO: 292-300.

c. Ds-SENSE Regions

As disclosed herein, an iADAR in the ON state can edit a SENSE codon in a RNA transcript, where the SENSE codon is located a double stranded region in the transcript, and where the iADAR can bind to the double stranded region to mutate the SENSE codon encoding a first amino acid into a mutated sense codon encoding a second amino acid. In some embodiments, the iADAR can bind to the double stranded region to mutate the SENSE codon encoding an amino acid into a mutated sense codon encoding the same amino acid. In some embodiments, the mutated sense codon can affect the activity of the RNA, e.g., splicing, translation, degradation, etc. Accordingly, in some embodiments, the SENSE codon is located in double stranded region, herein referred to as “ds-SENSE” region. In some embodiments, the ds-SENSE region is a short hairpin loop, where the short hairpin loop is RNA or mRNA.

In some embodiments, the ds-SENSE region is a double stranded RNA transcript that comprises (i) a SENSE codon as disclosed herein, and (ii) a Binding motif for RBD (BM), where the Binding motif for RBD (BM) binds to a RNA-binding domain (RBD) of the iADAR. In some embodiments, the Binding motif for RBD (BM) is capable of being bound by an RNA-binding domain of the DD.

In some embodiments, the ds-SENSE region comprises at least one hairpin. In some embodiments, the ds-SENSE region comprises at least one hairpin comprising the at least one sense codon and the Binding motif for RBD (BM). In some embodiments, the ds-SENSE region comprises a first hairpin comprising the at least one SENSE codon and a second hairpin comprising the Binding motif for RBD (BM).

As disclosed herein, in the presence of an inducer the iADAR changes an A to an I in mRNA. In some embodiments, the ds-SENSE region comprises a codon selected from Table 15, which is edited to a mutated sense codon in the presence of an inducer, as shown in Table 15. In some embodiments, the sense codon comprises an adenosine nucleotide in the first position of the codon. In some embodiments, the sense codon comprises an adenosine nucleotide in the second position of the codon. In some embodiments, the sense codon comprises an adenosine nucleotide in the third position of the codon. In some embodiments, the sense codon comprises an adenosine nucleotide in the first and second positions of the codon. In some embodiments, the sense codon comprises an adenosine nucleotide in the second and third positions of the codon. In some embodiments, the sense codon comprises an adenosine nucleotide in the first and third positions of the codon. In some embodiments, the sense codon comprises an adenosine nucleotide in the first, second, and third positions of the codon.

In some embodiments, the sense codon is within a self-cleaving peptide sequence. In some embodiments, mutation of the sense codon to the mutated codon results in increased or decreased cleavage of the self-cleaving peptide when in the translated protein. In some embodiments, the self-cleaving peptide belongs to the 2A peptide family, which can also be referred to as a 2A Ribosomal Skip Sequence. Non-limiting examples of 2A peptides include P2A, E2A, F2A and T2A (see e.g., SEQ ID NOs 360-363). F2A is derived from foot-and-mouth disease virus 18; E2A is derived from equine rhinitis A virus; P2A is derived from porcine teschovirus-1 2A; T2A is derived from thosea asigna virus 2A. In some embodiments of any of the aspects, the N-terminal of the 2A peptide comprises the sequence “GSG” (Gly-Ser-Gly). In some embodiments of any of the aspects, the N-terminal of the 2A peptide does not comprise the sequence “GSG” (Gly-Ser-Gly).

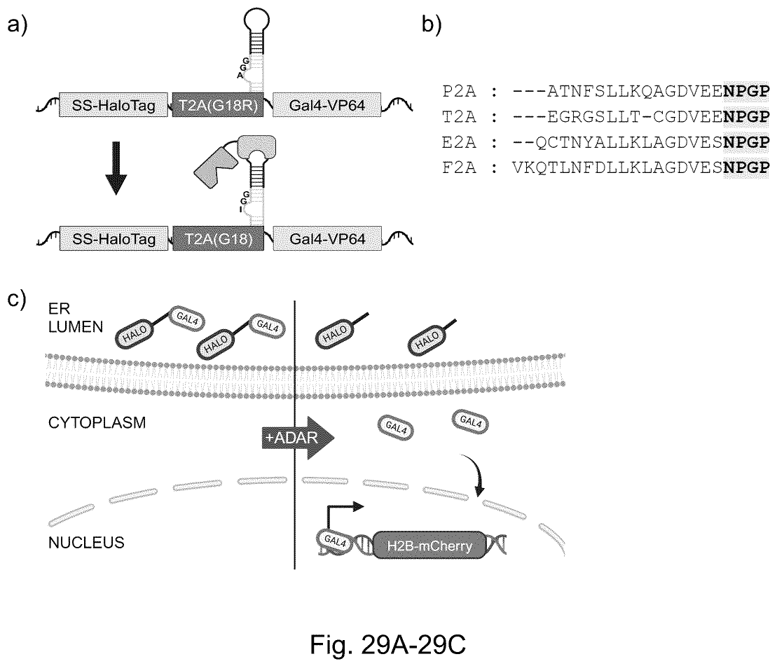

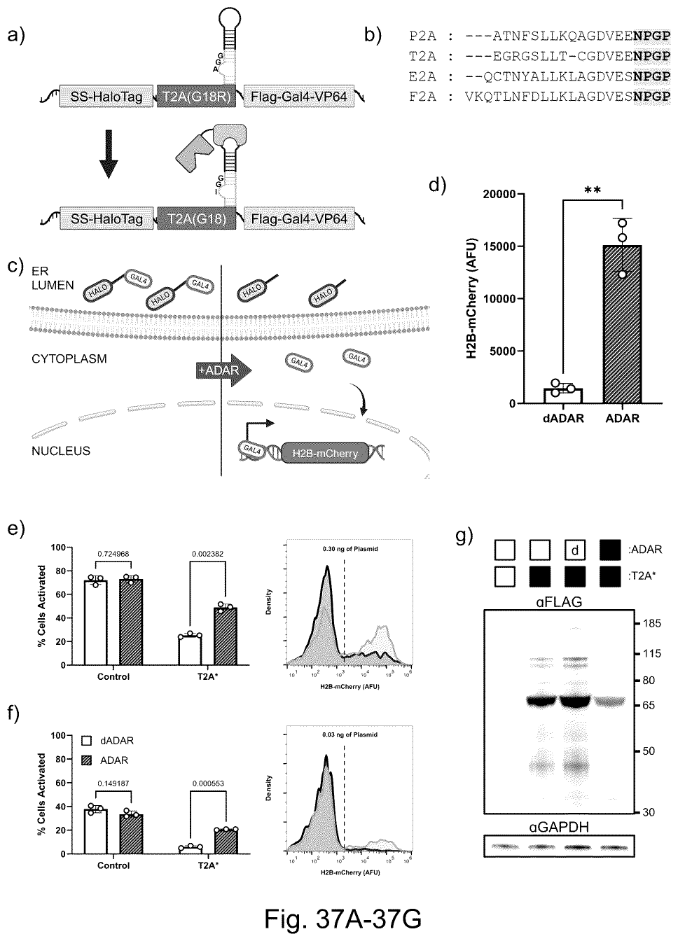

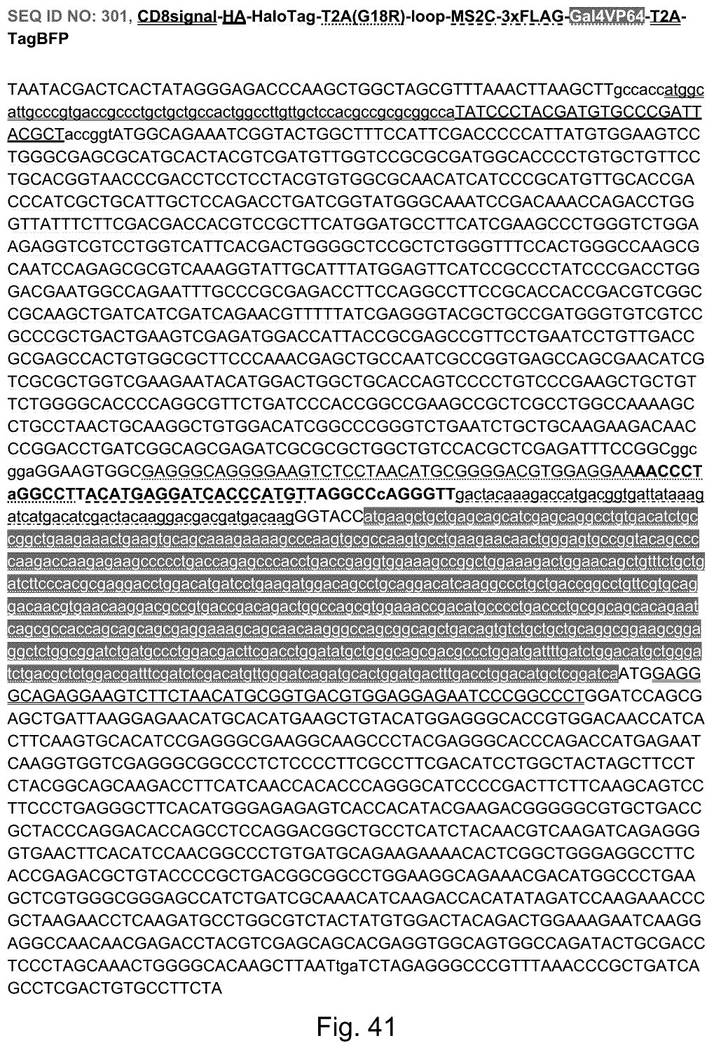

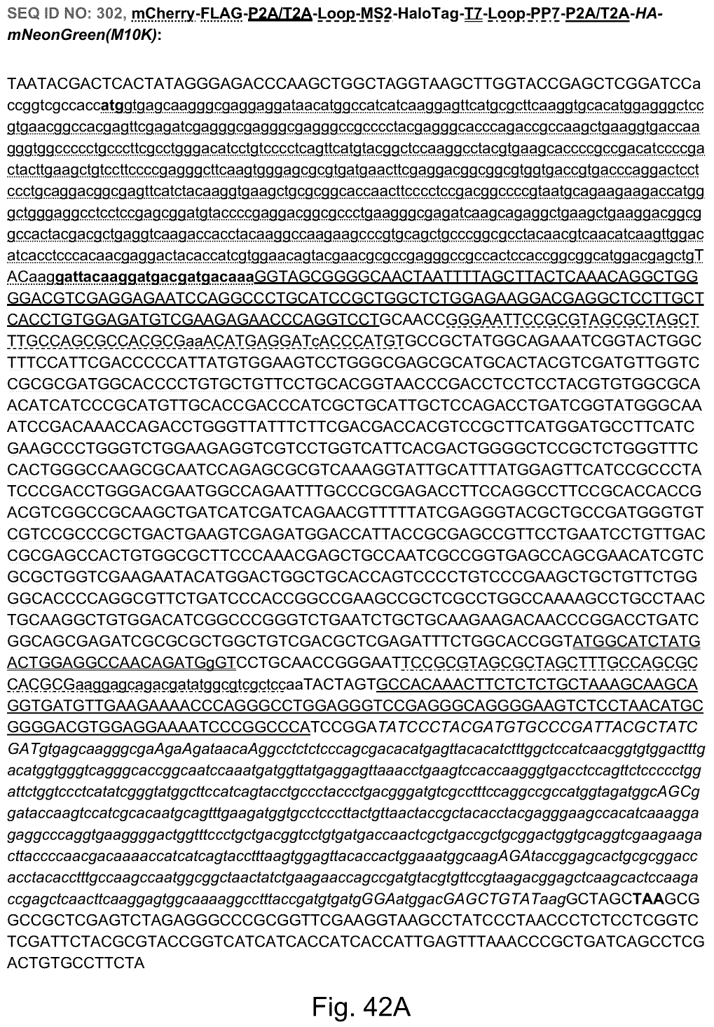

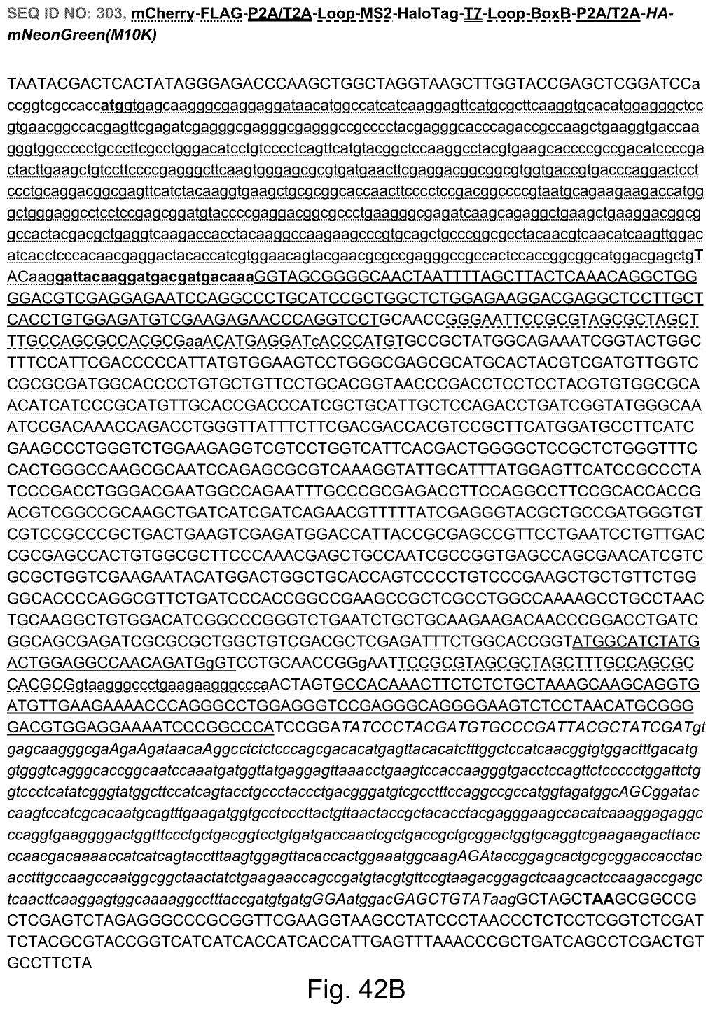

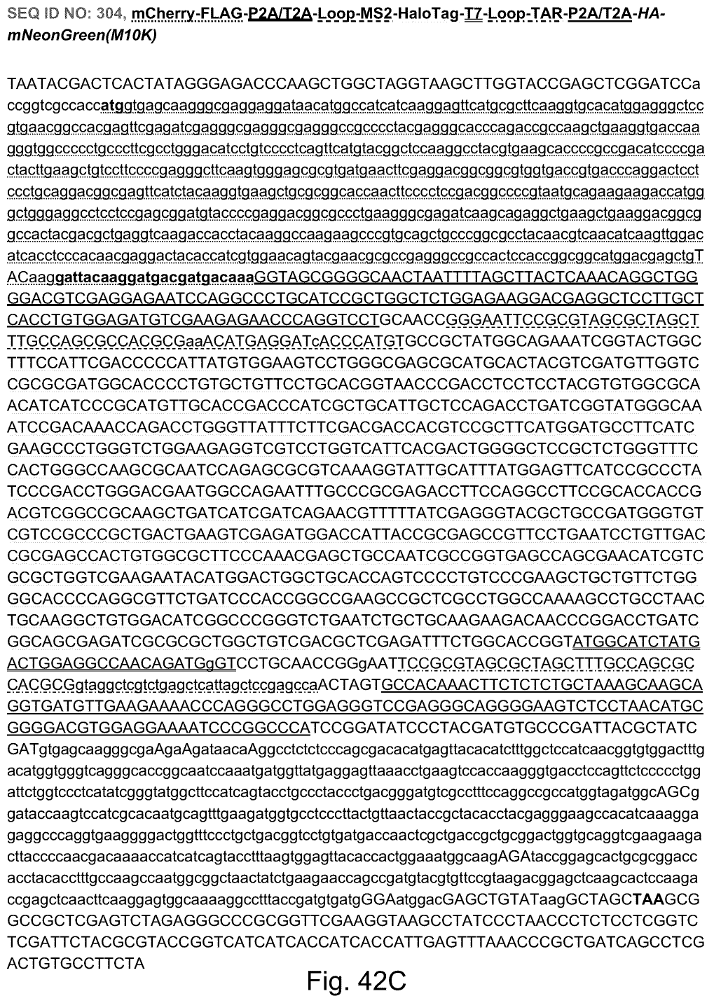

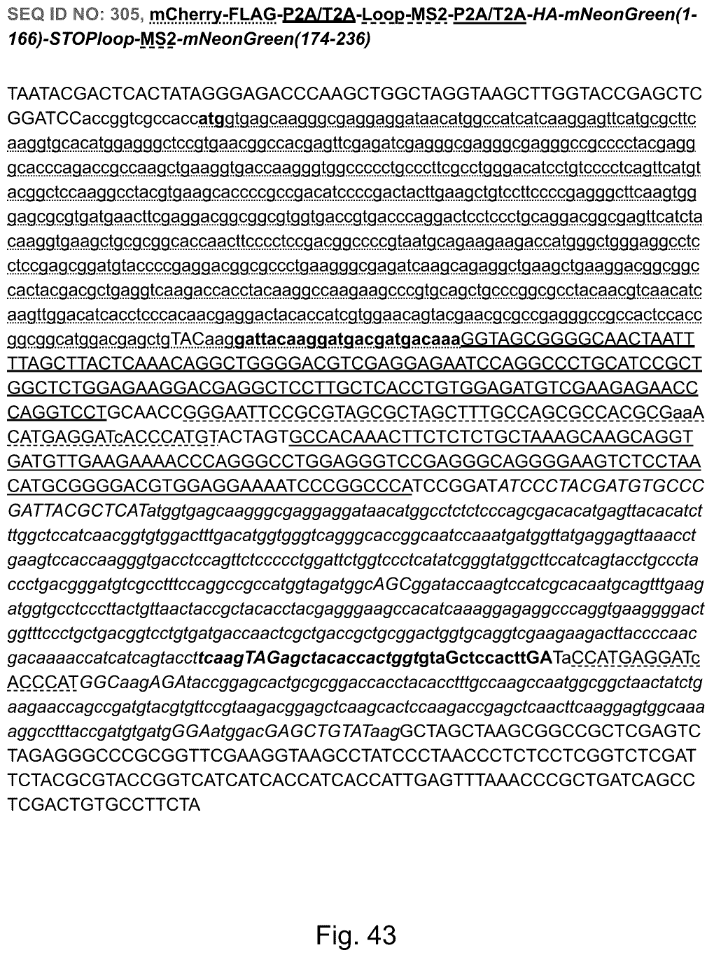

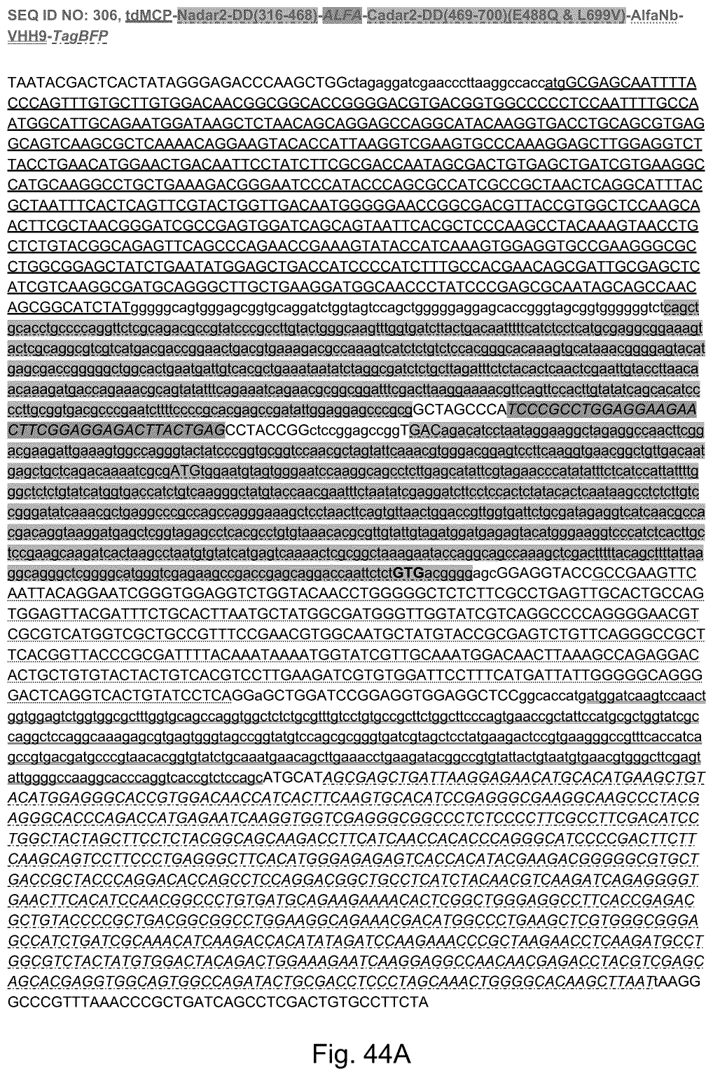

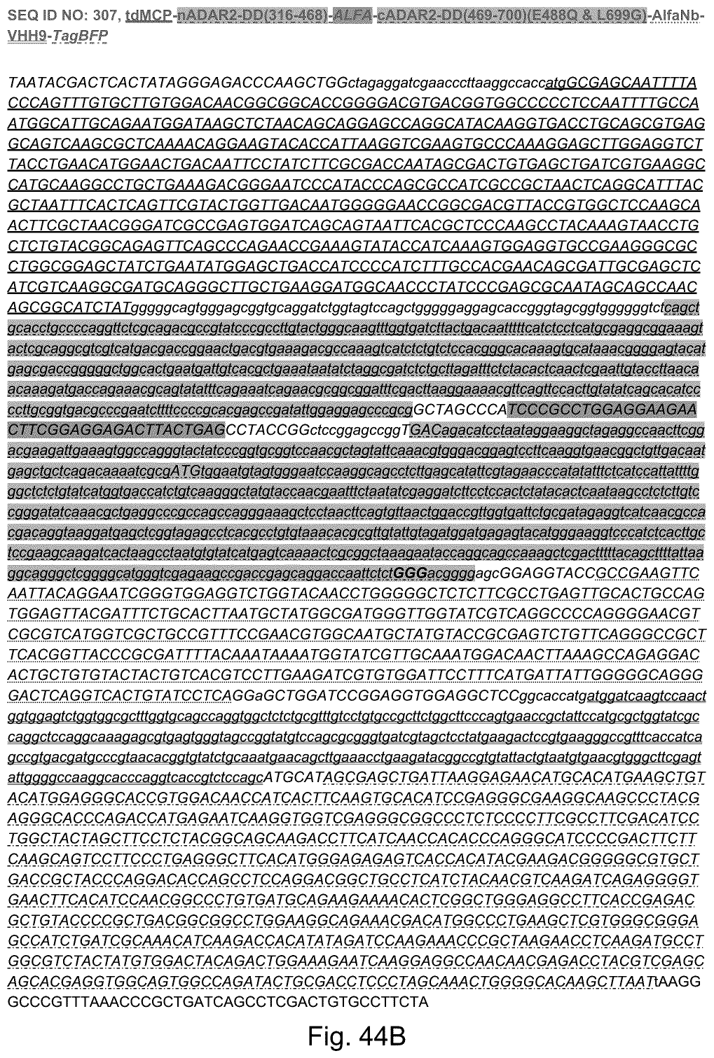

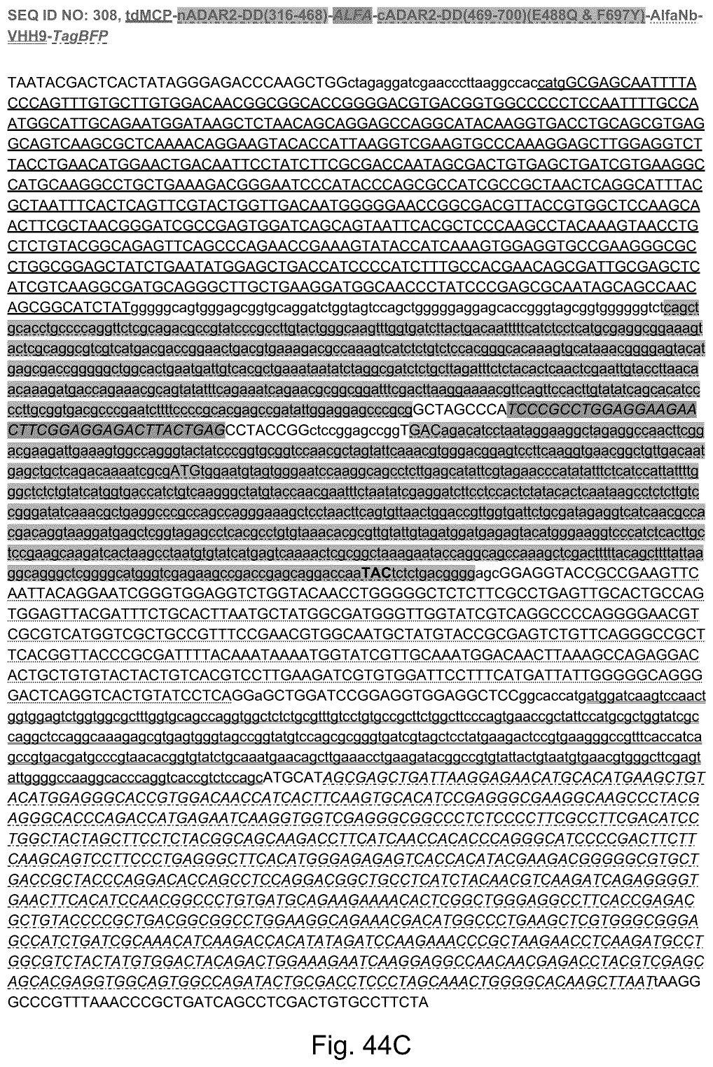

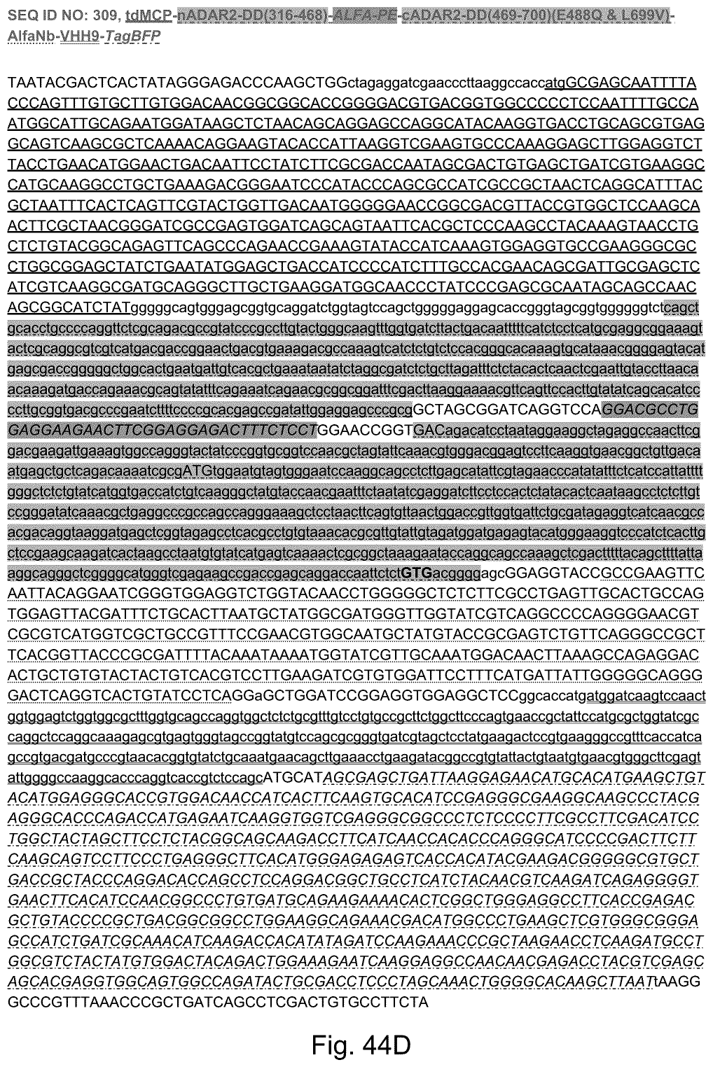

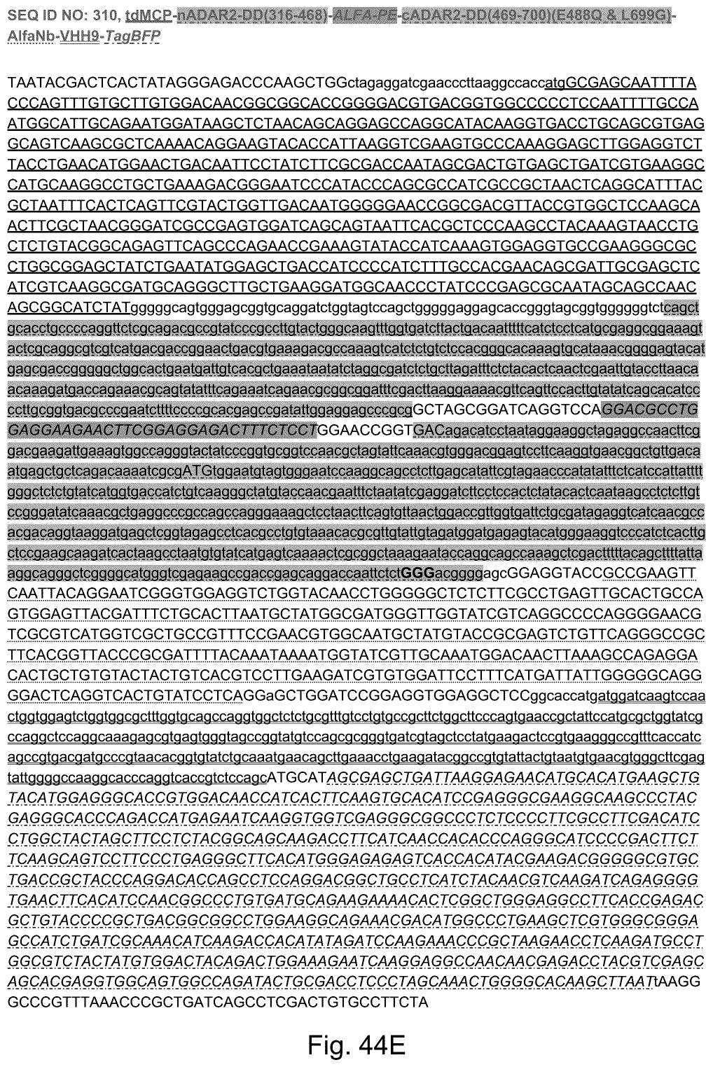

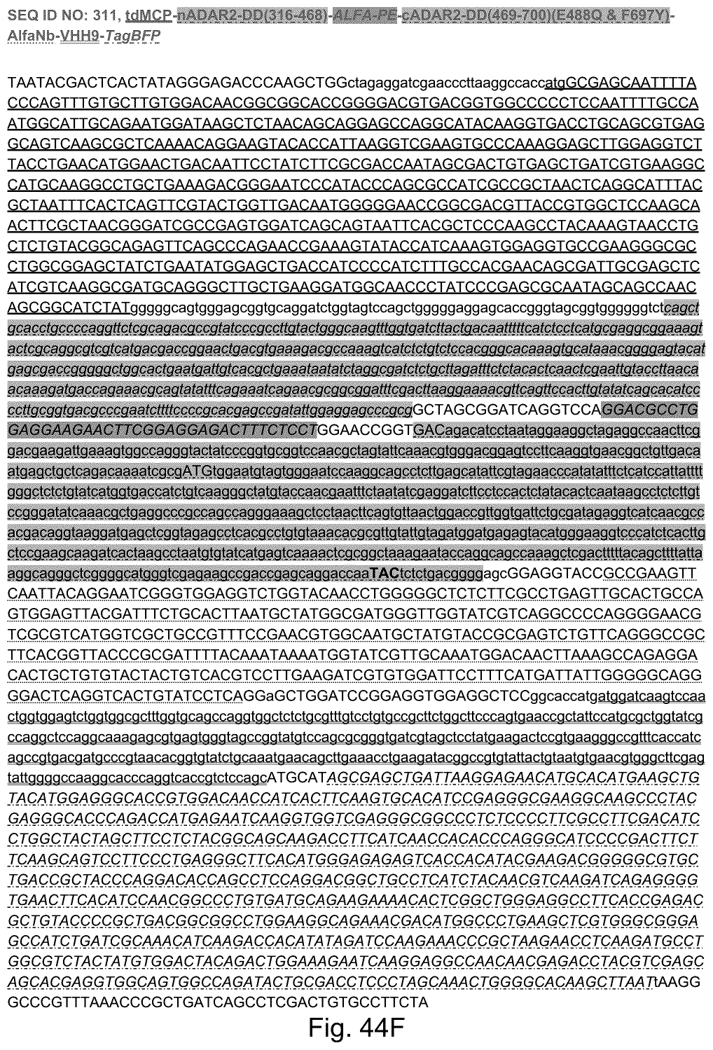

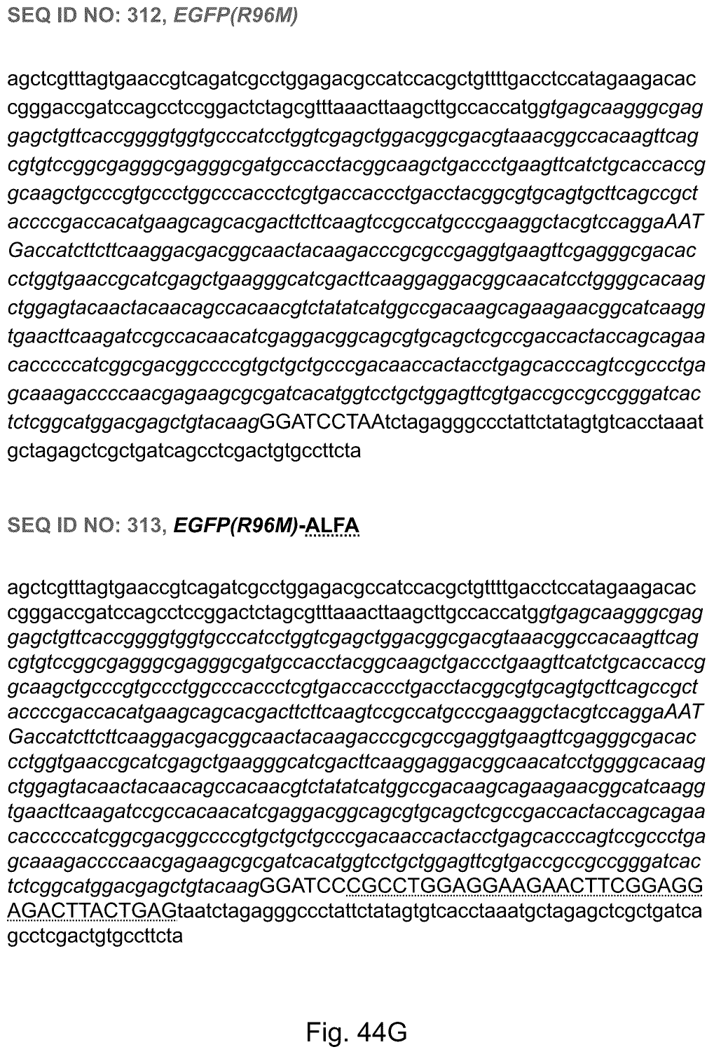

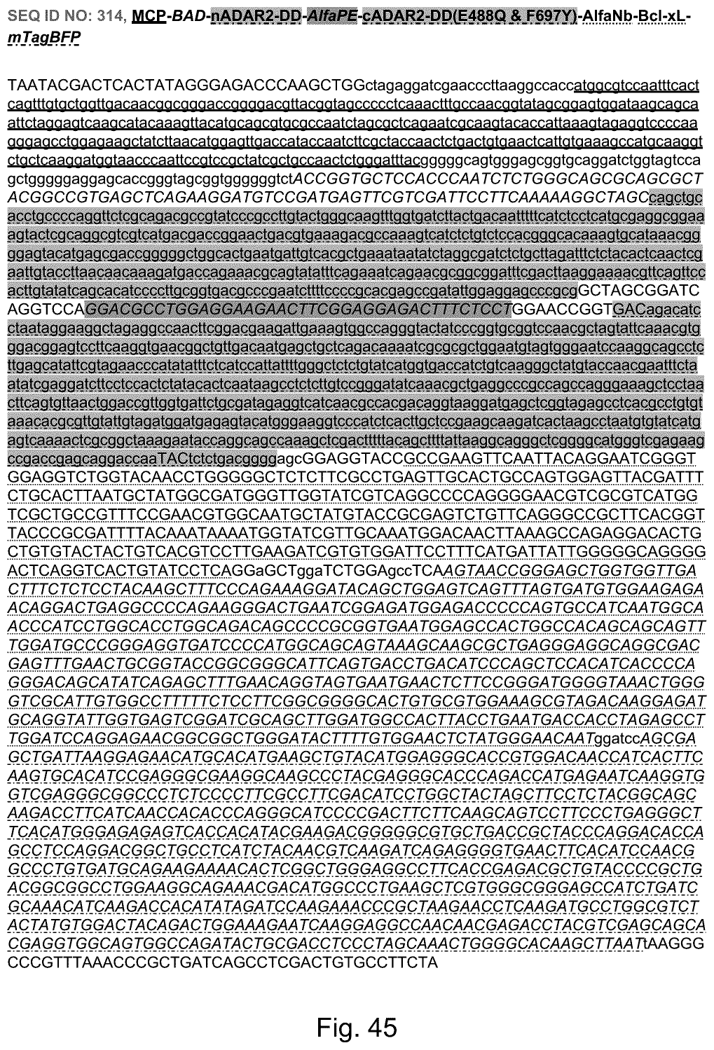

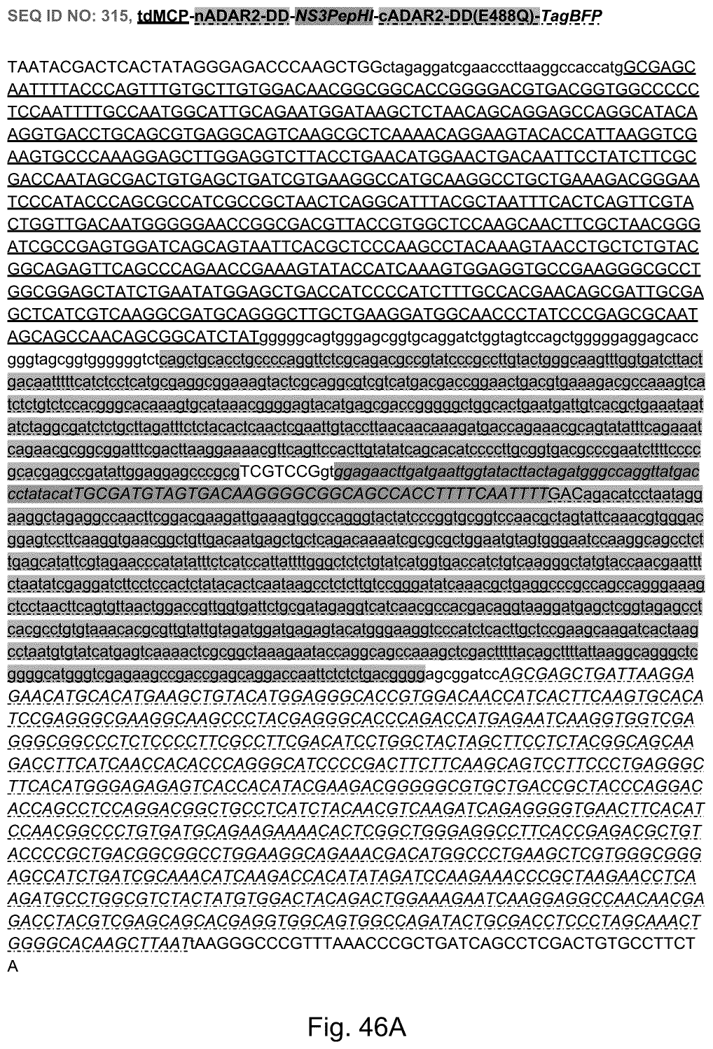

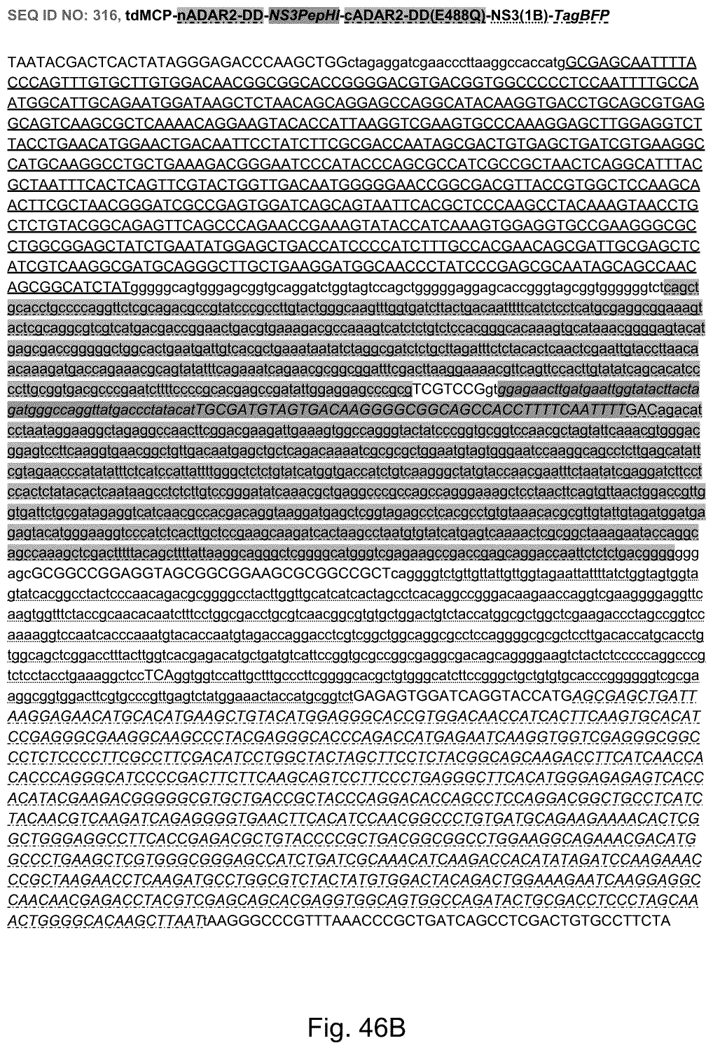

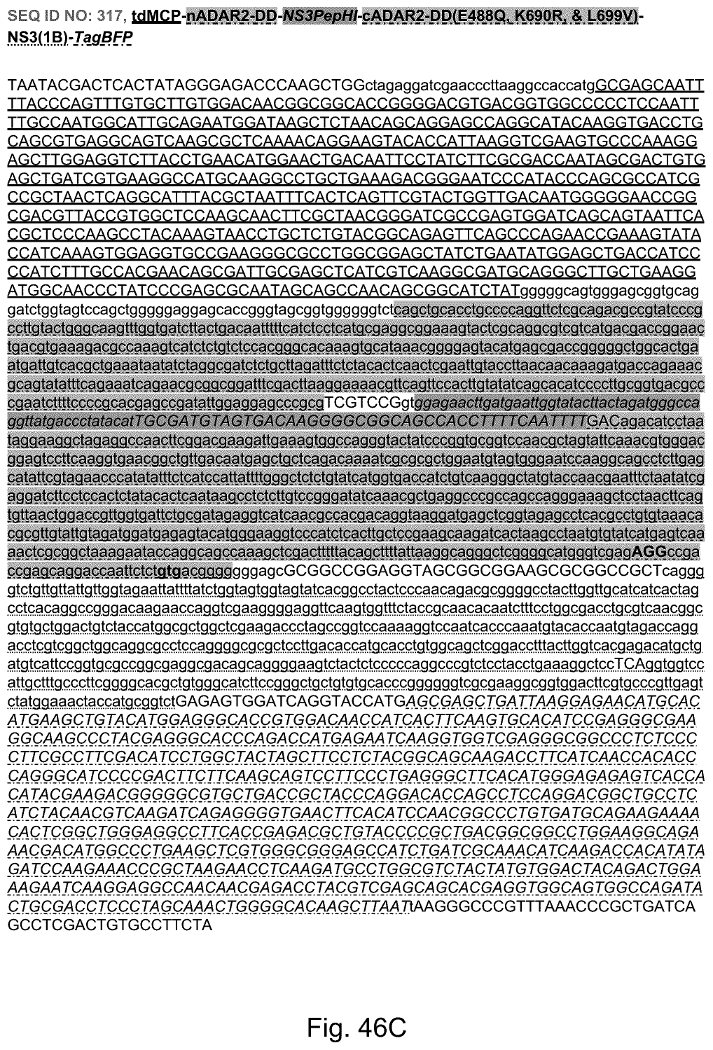

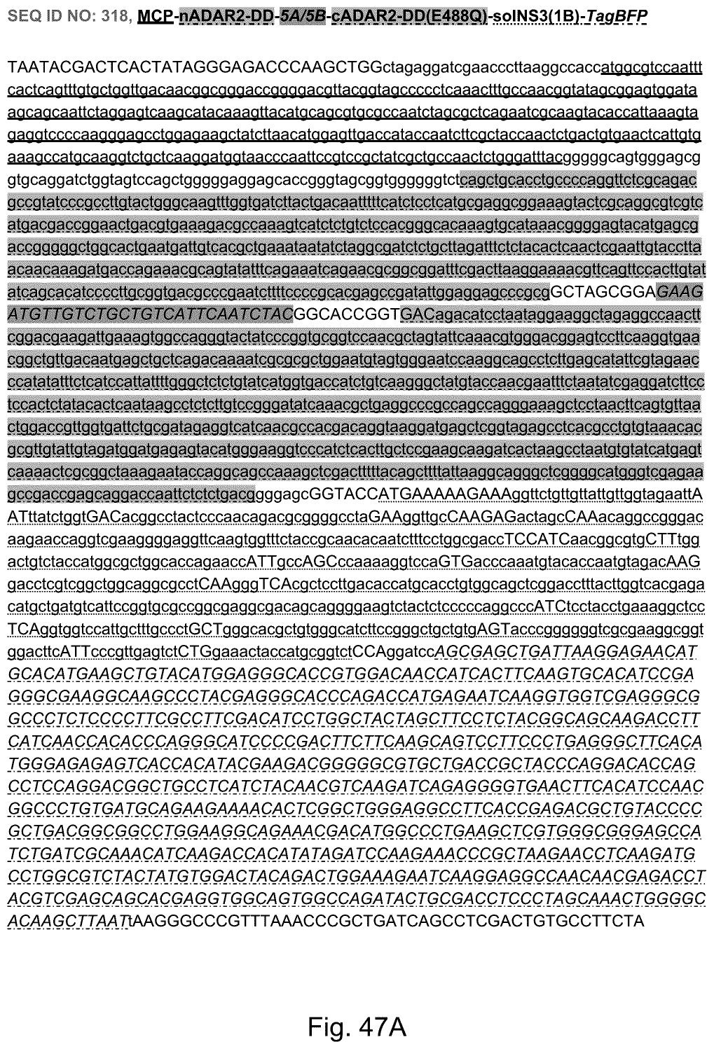

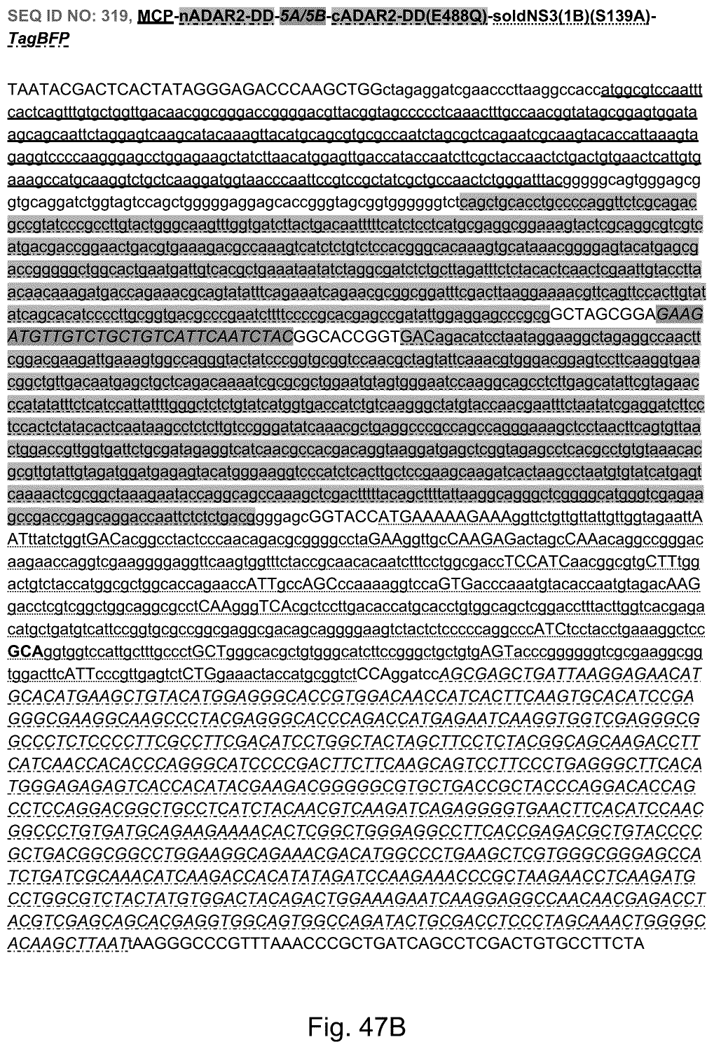

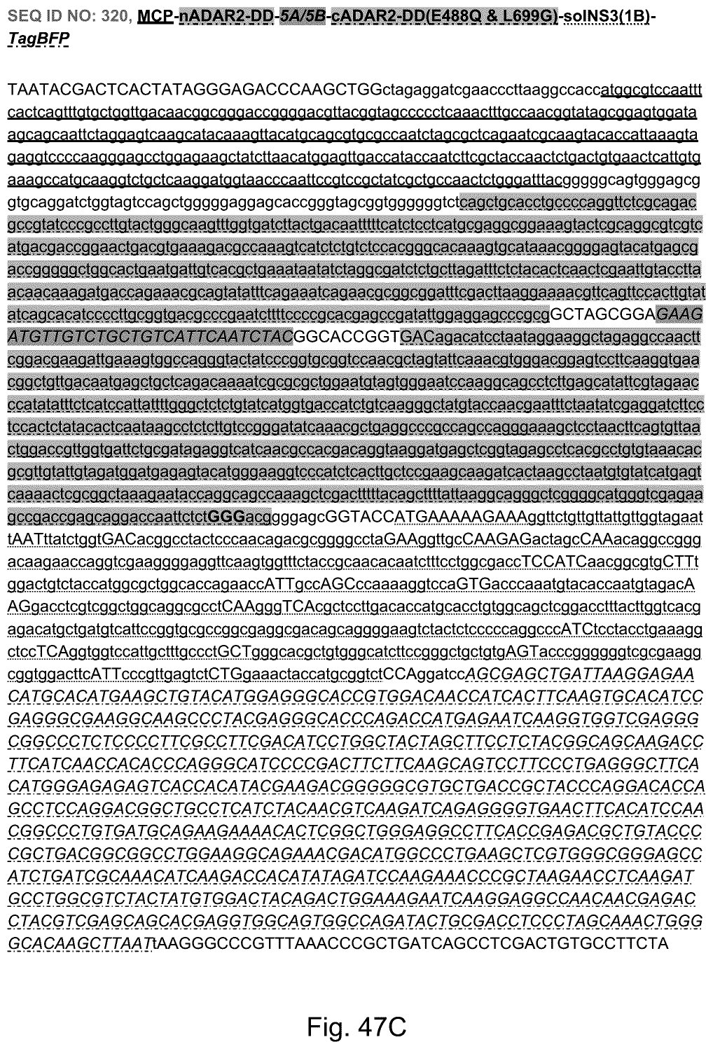

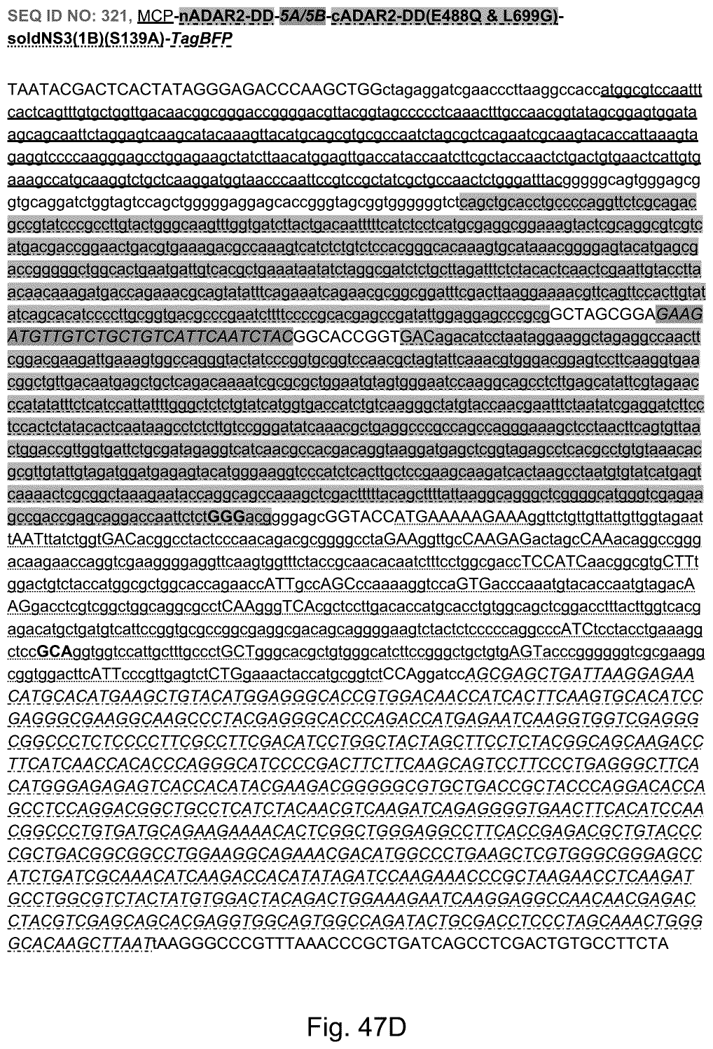

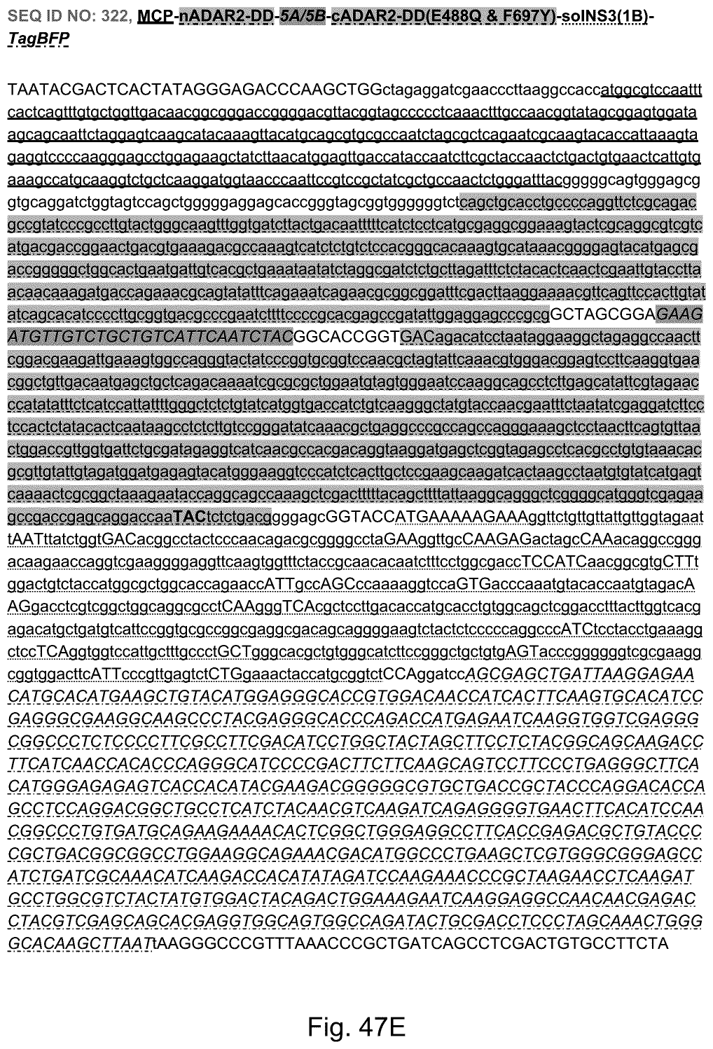

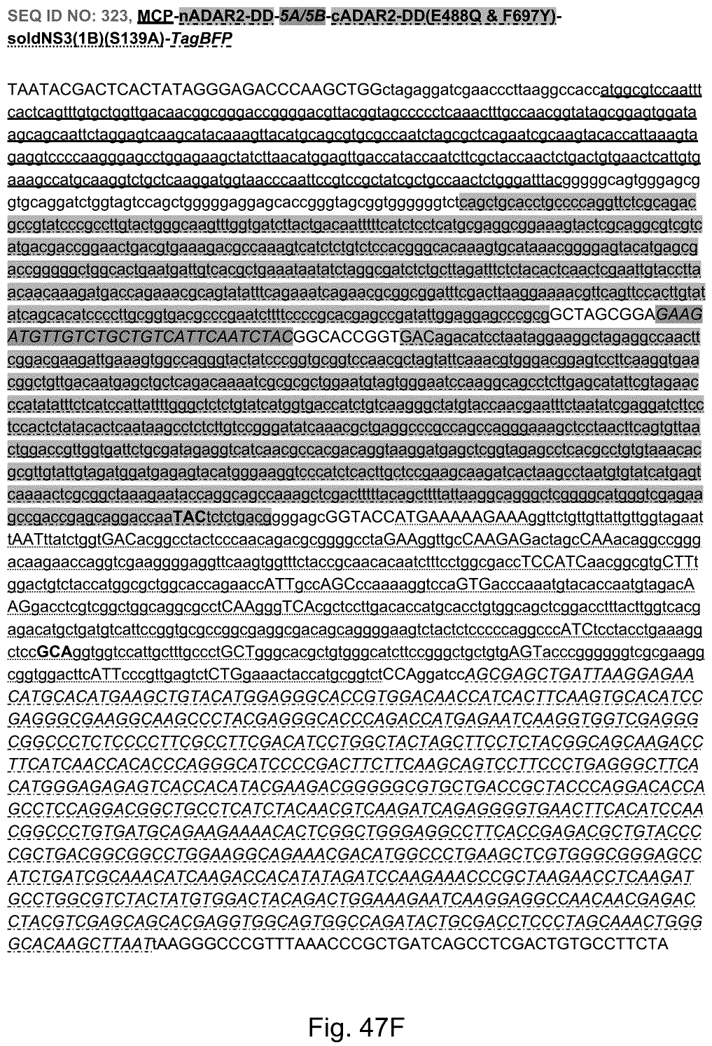

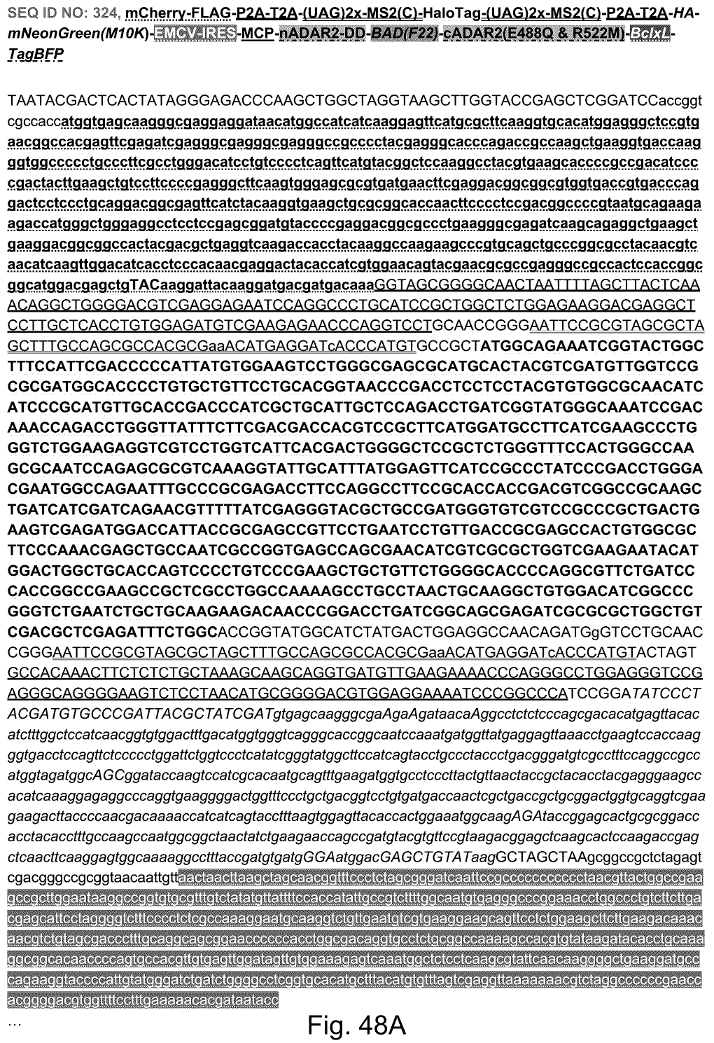

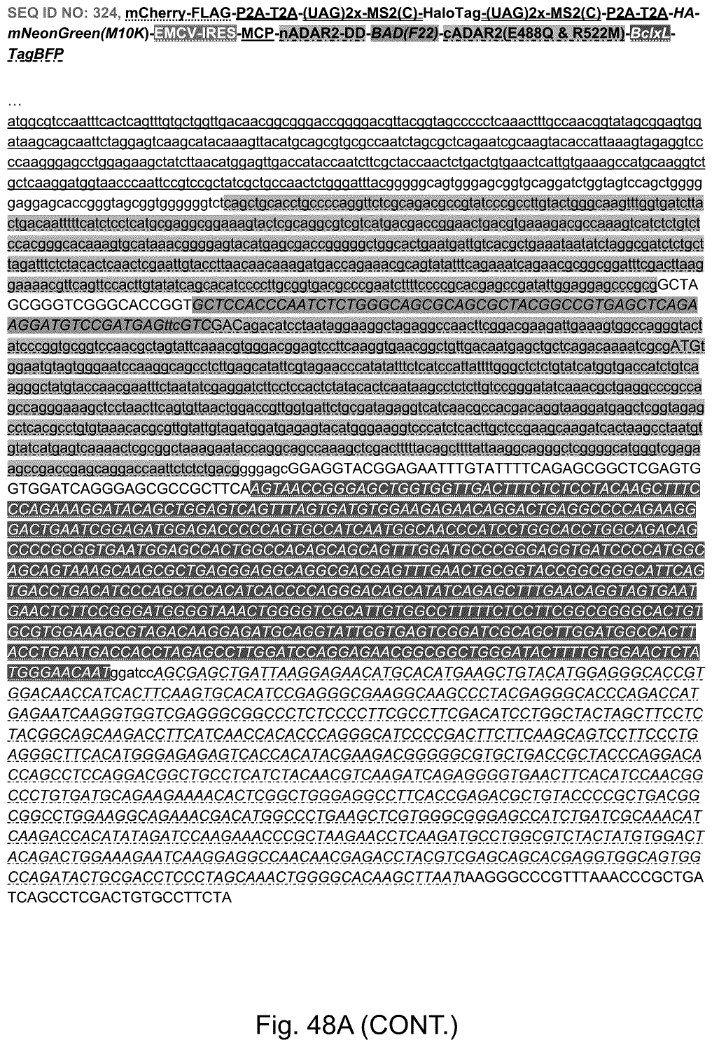

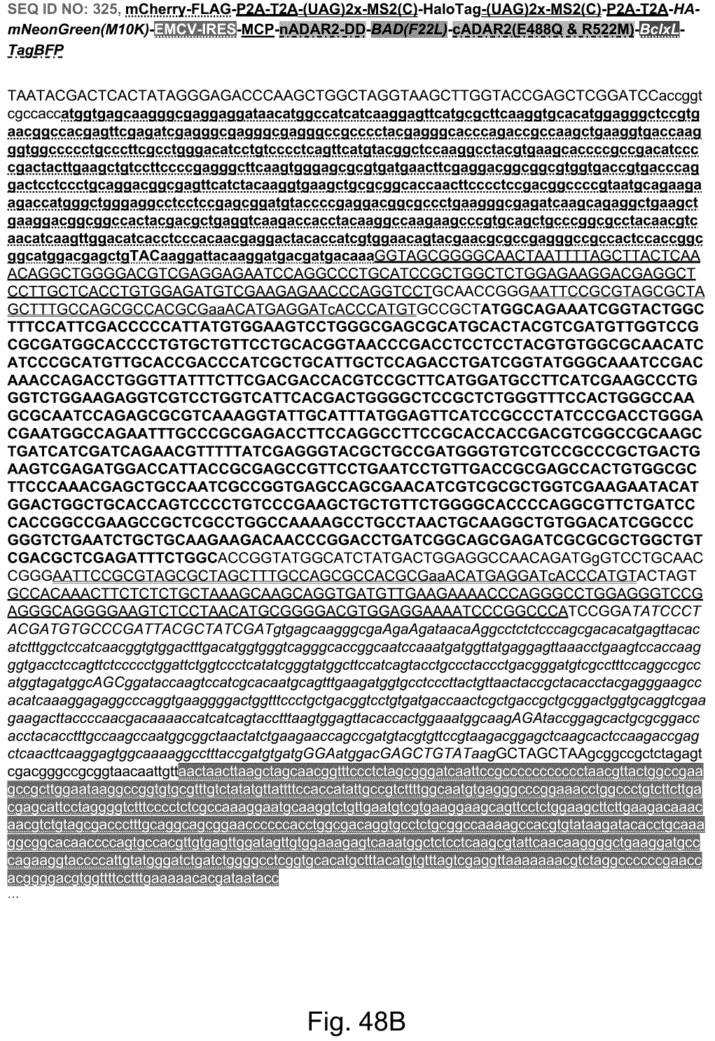

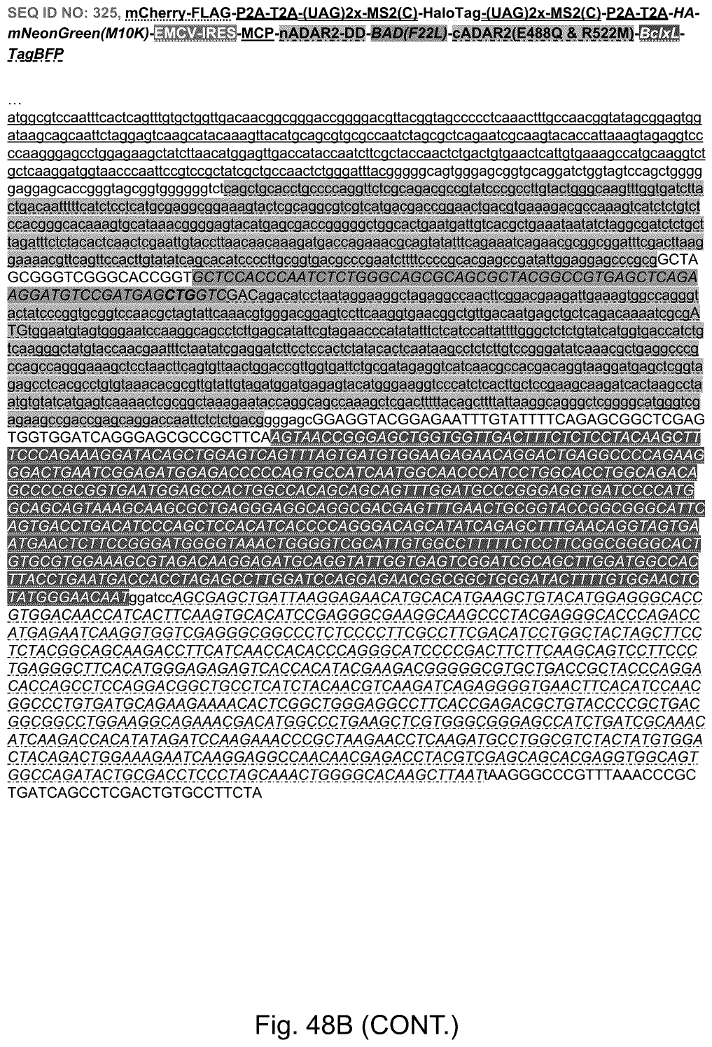

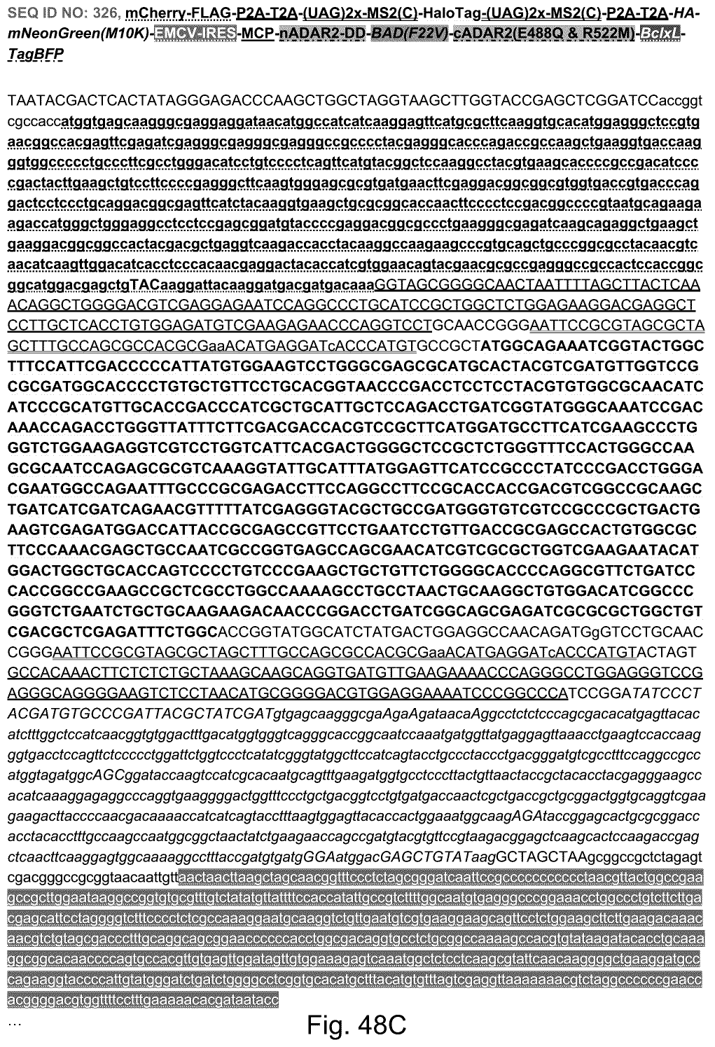

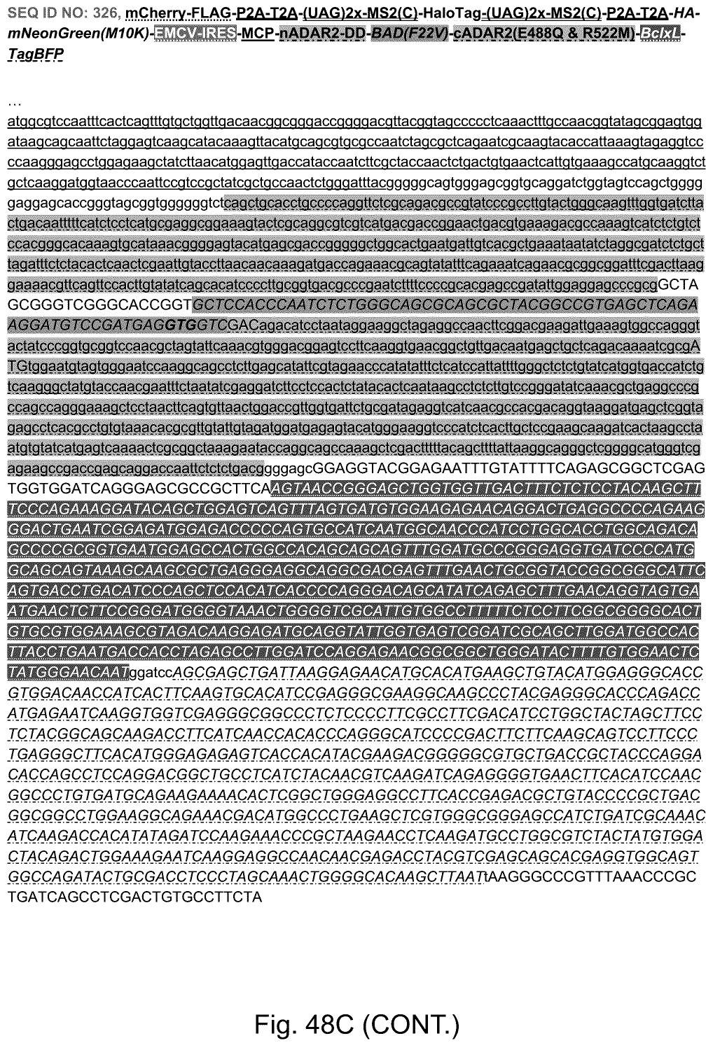

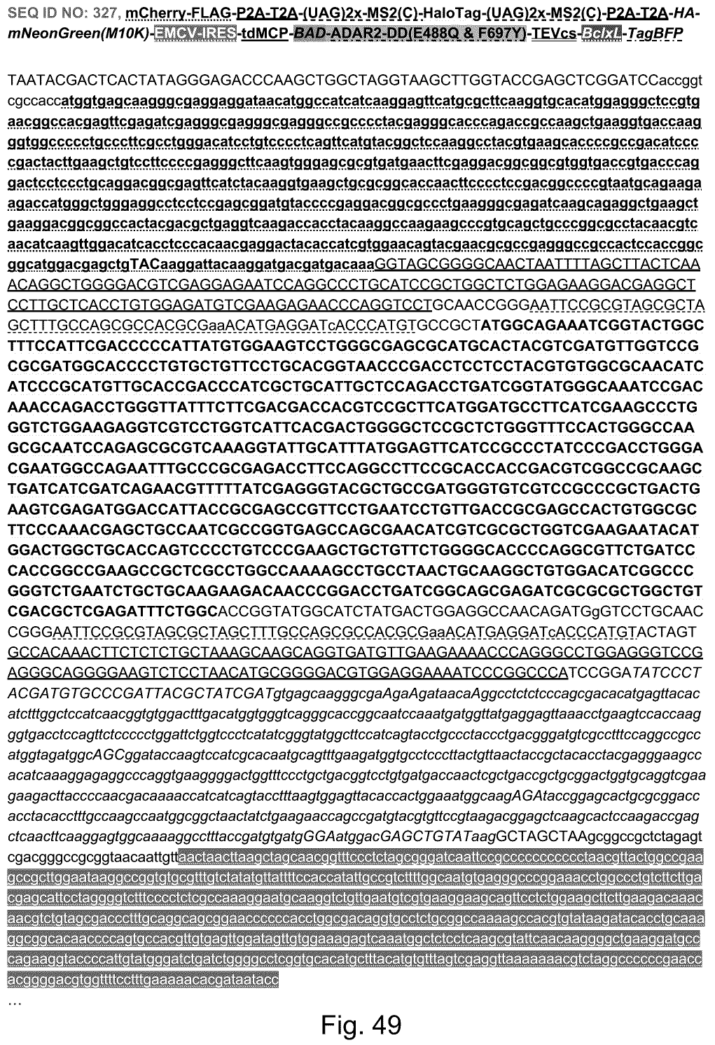

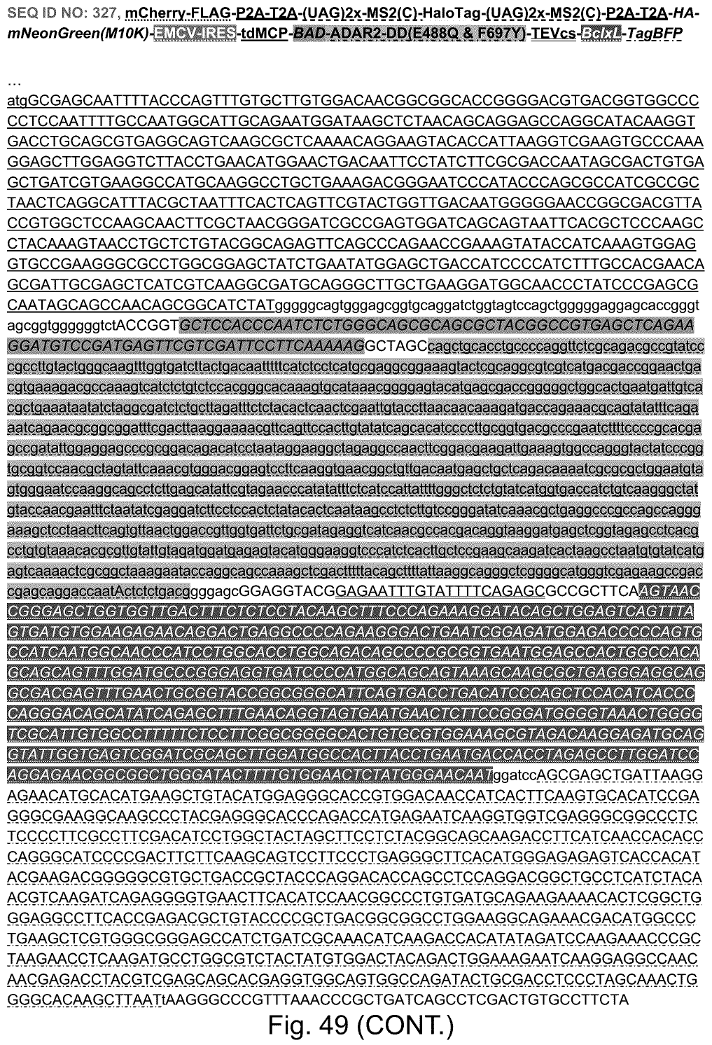

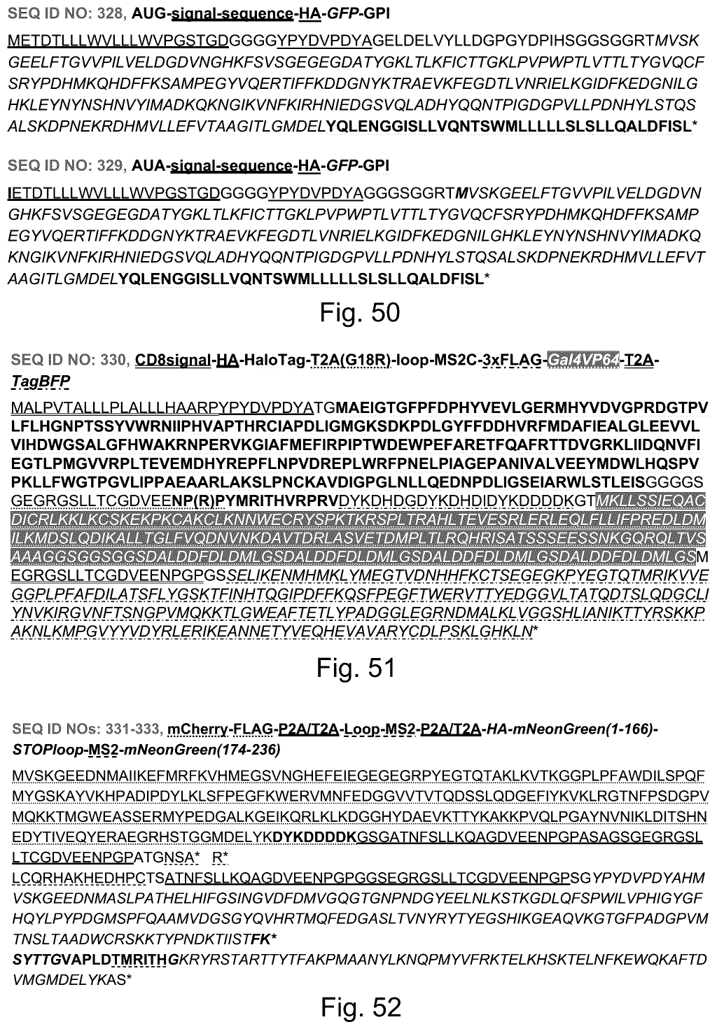

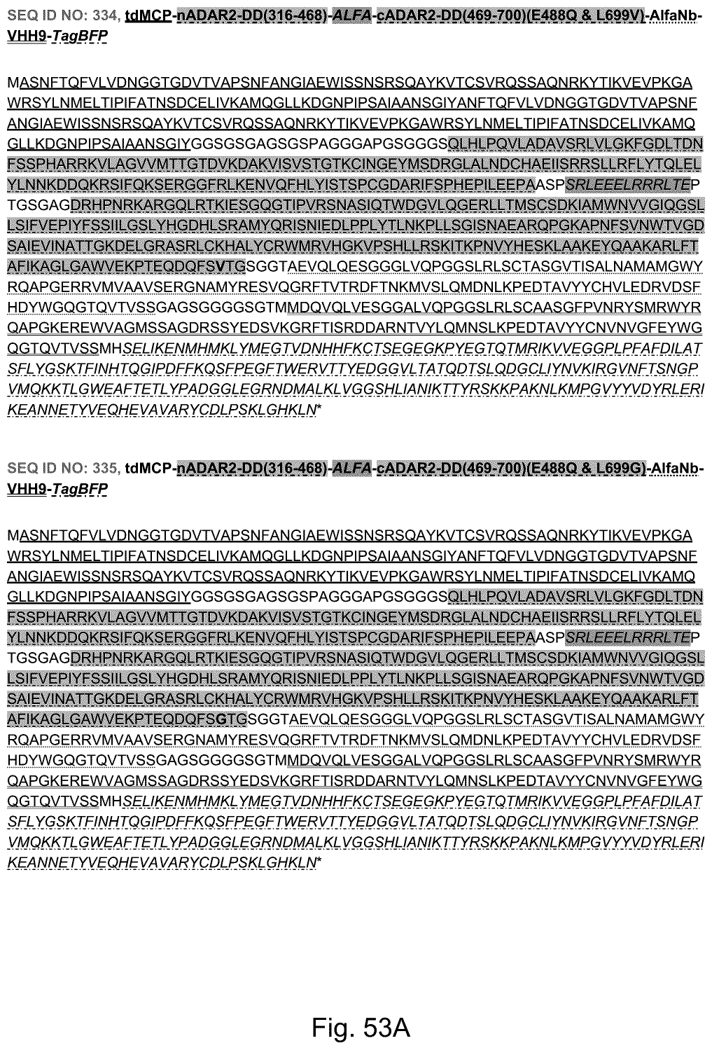

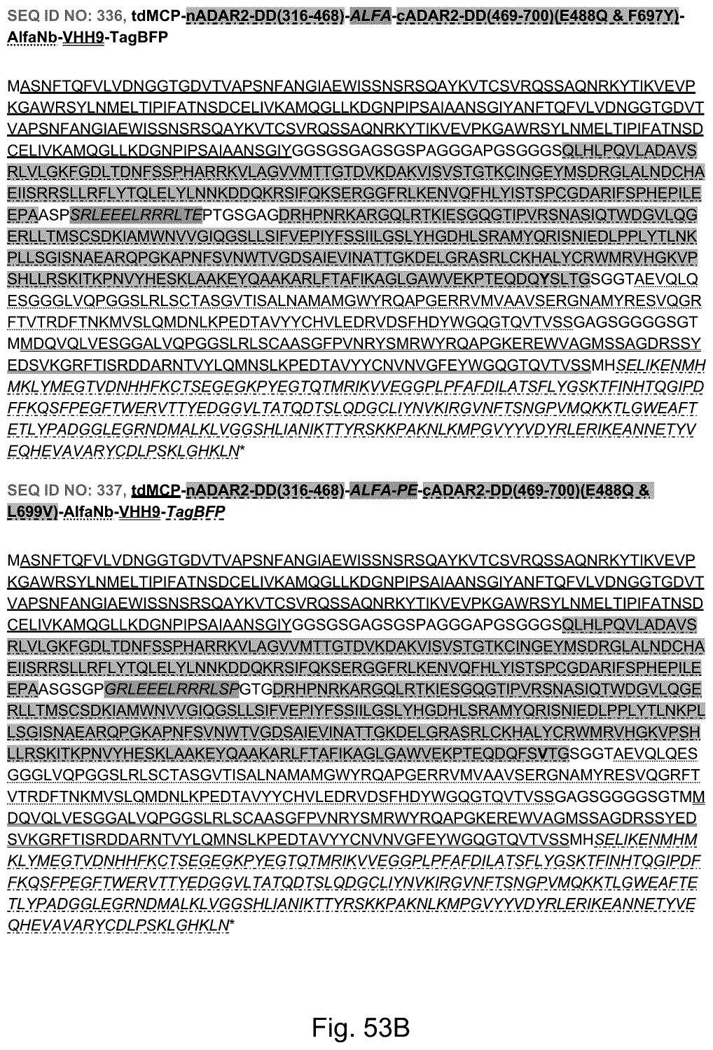

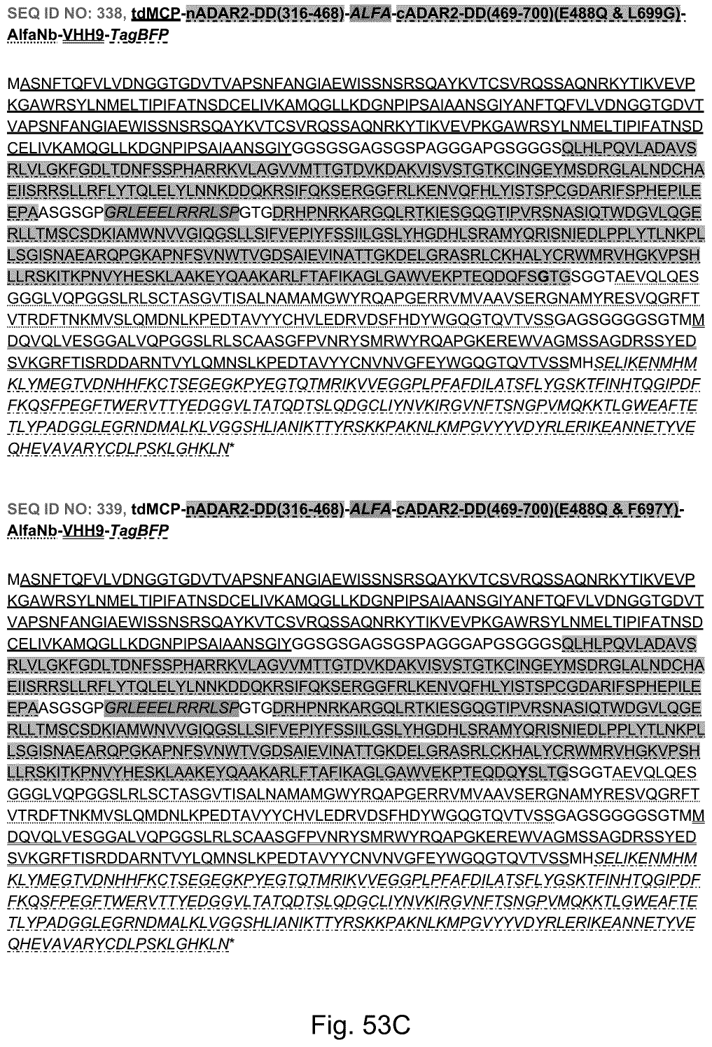

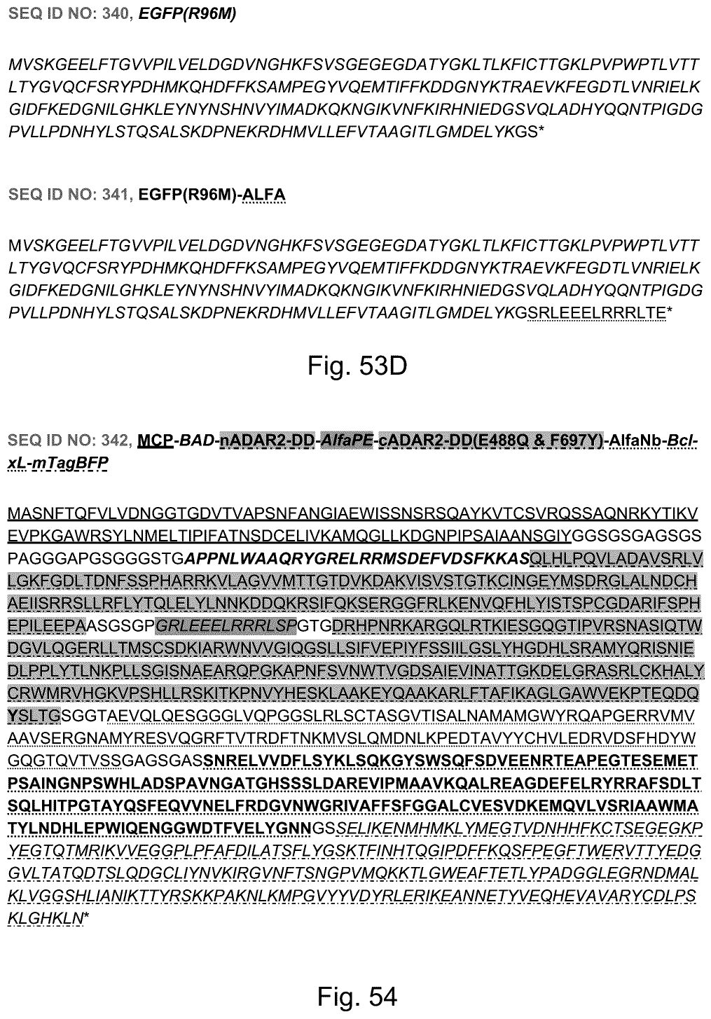

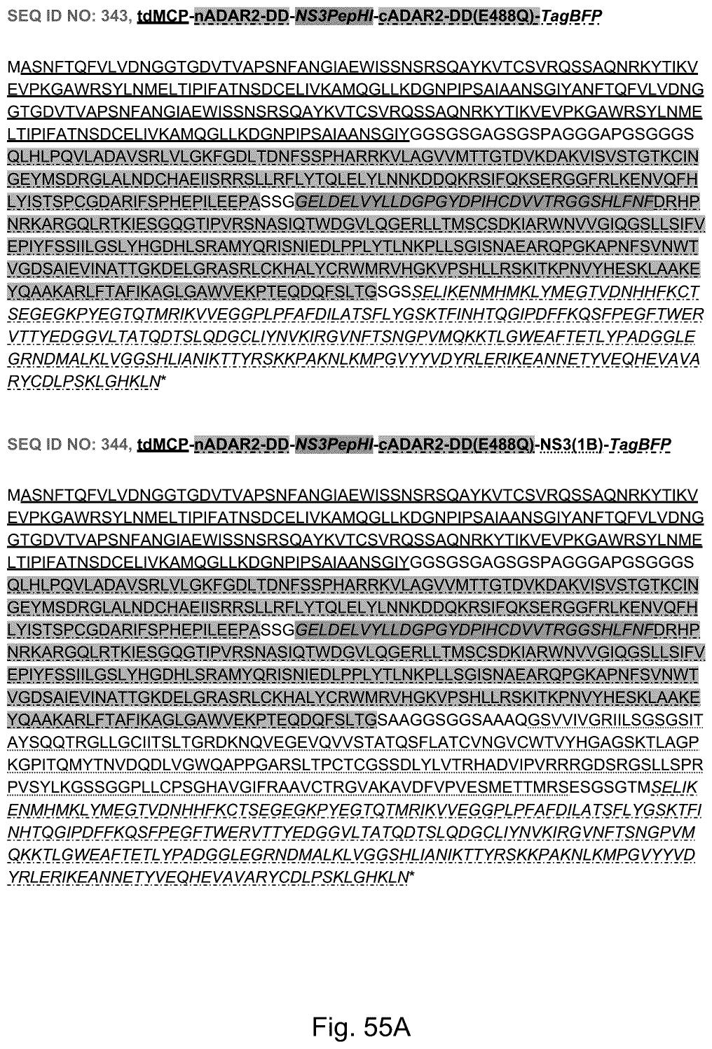

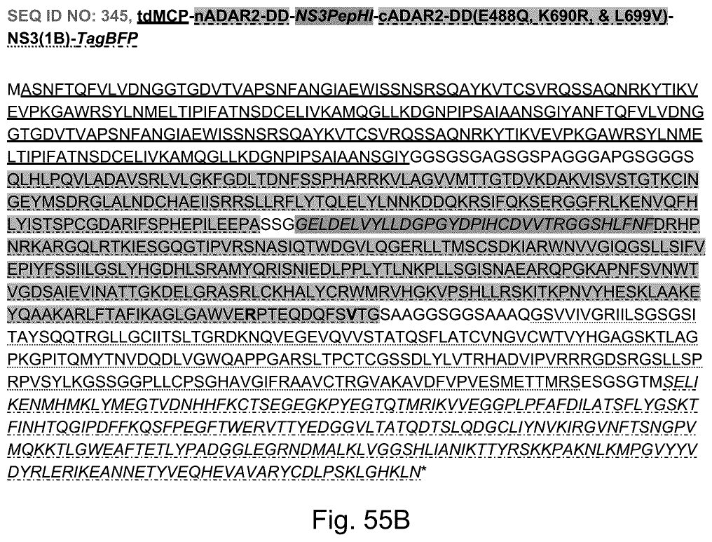

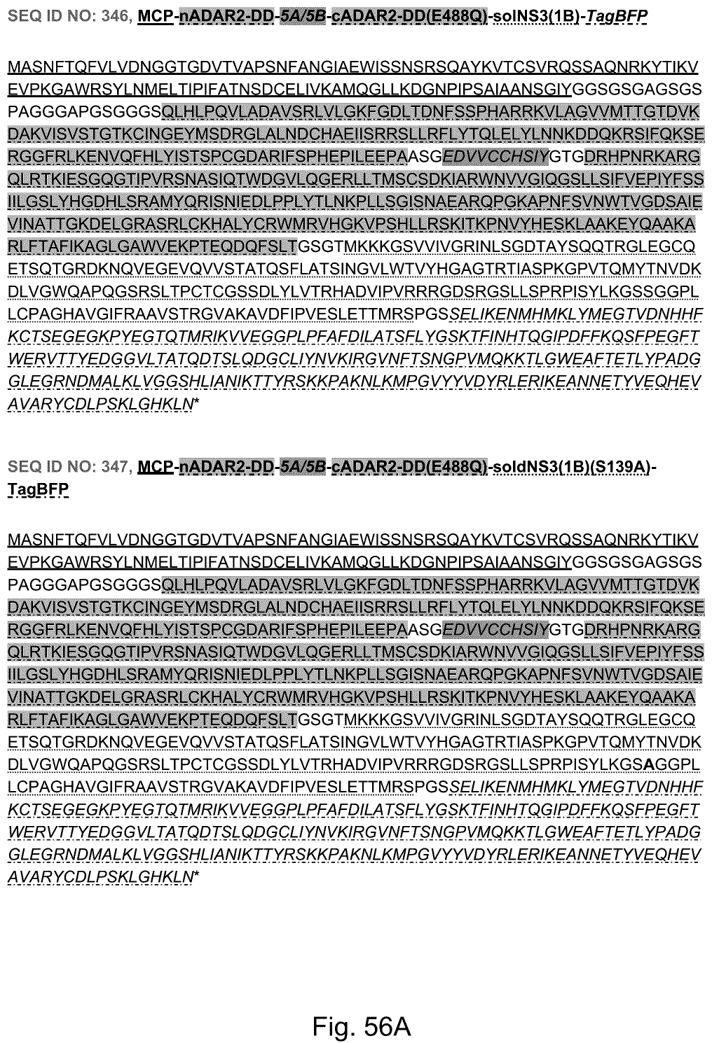

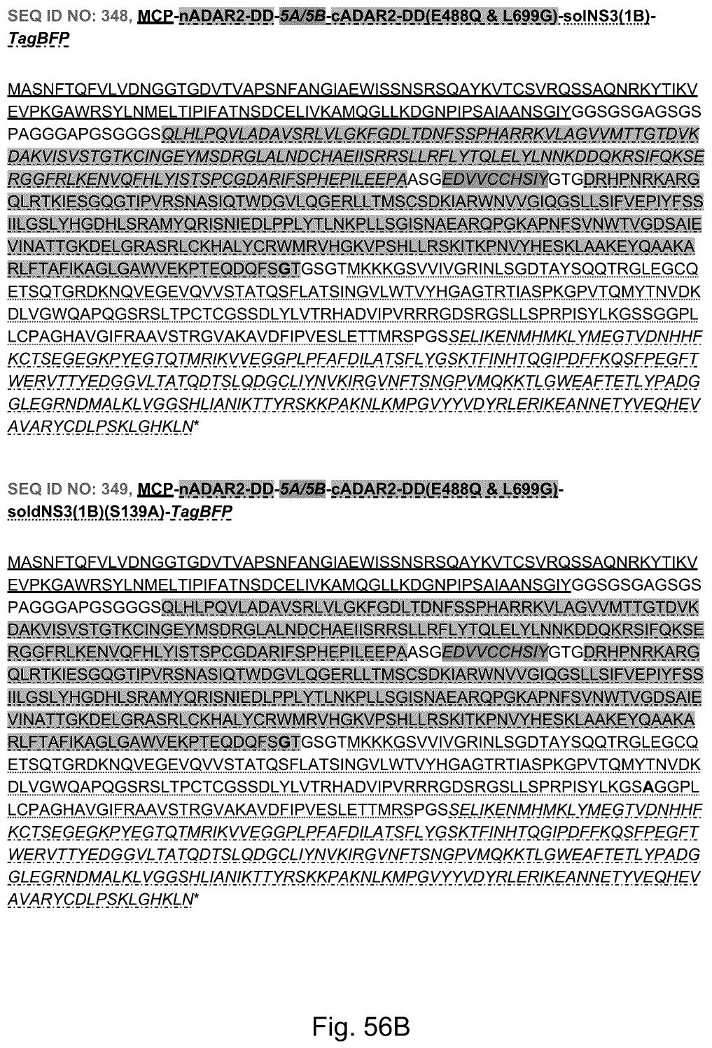

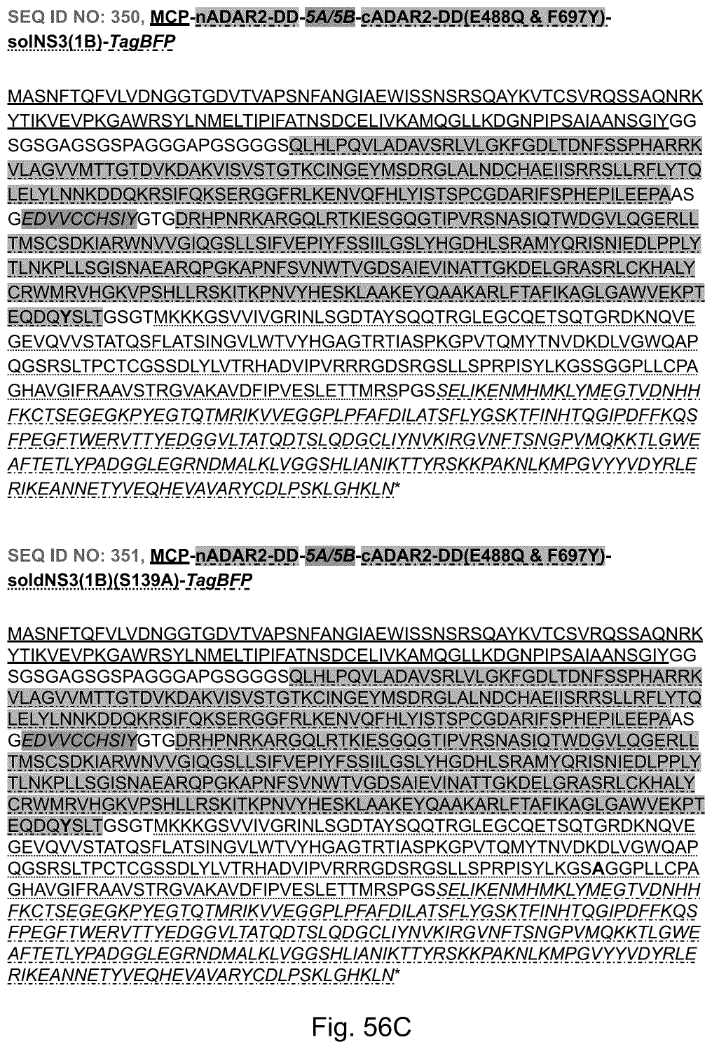

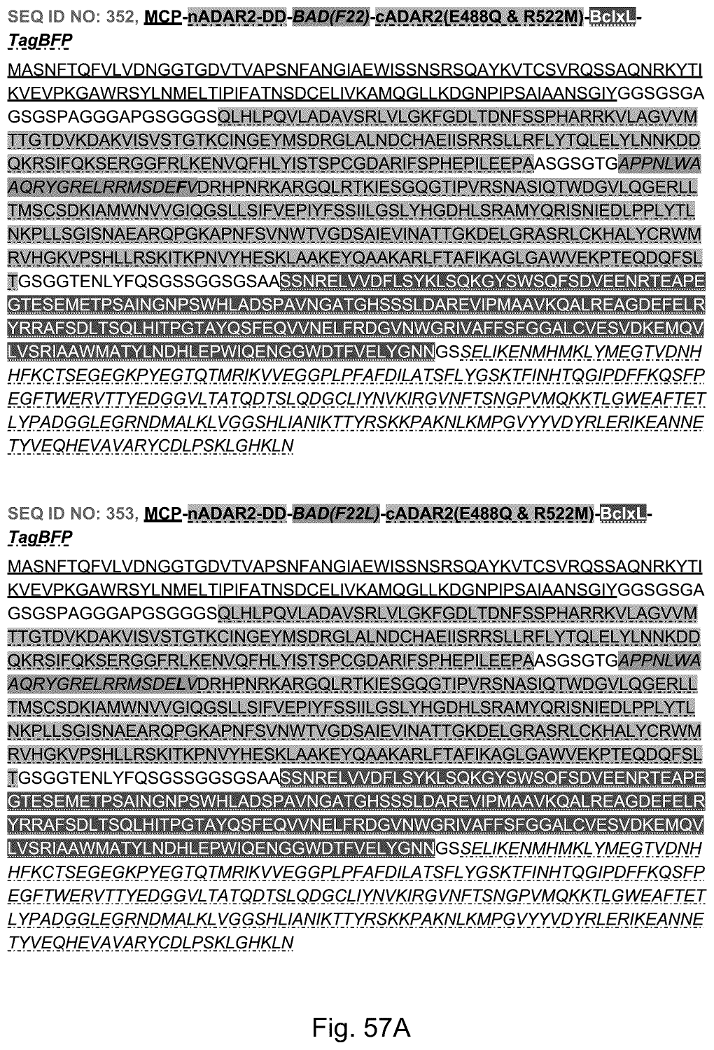

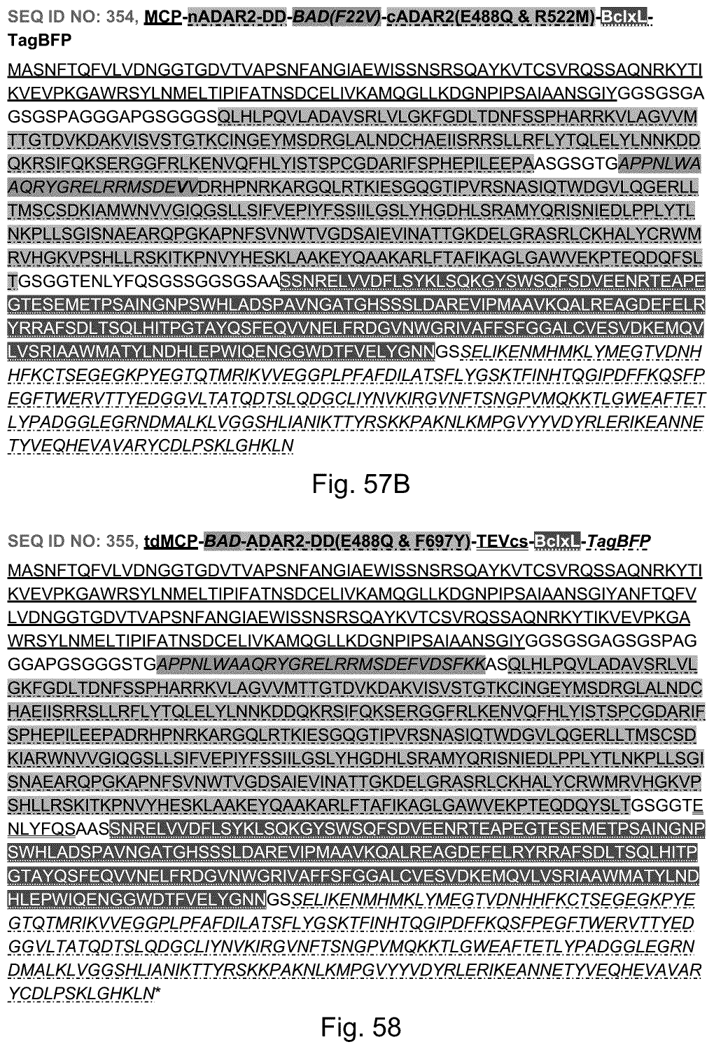

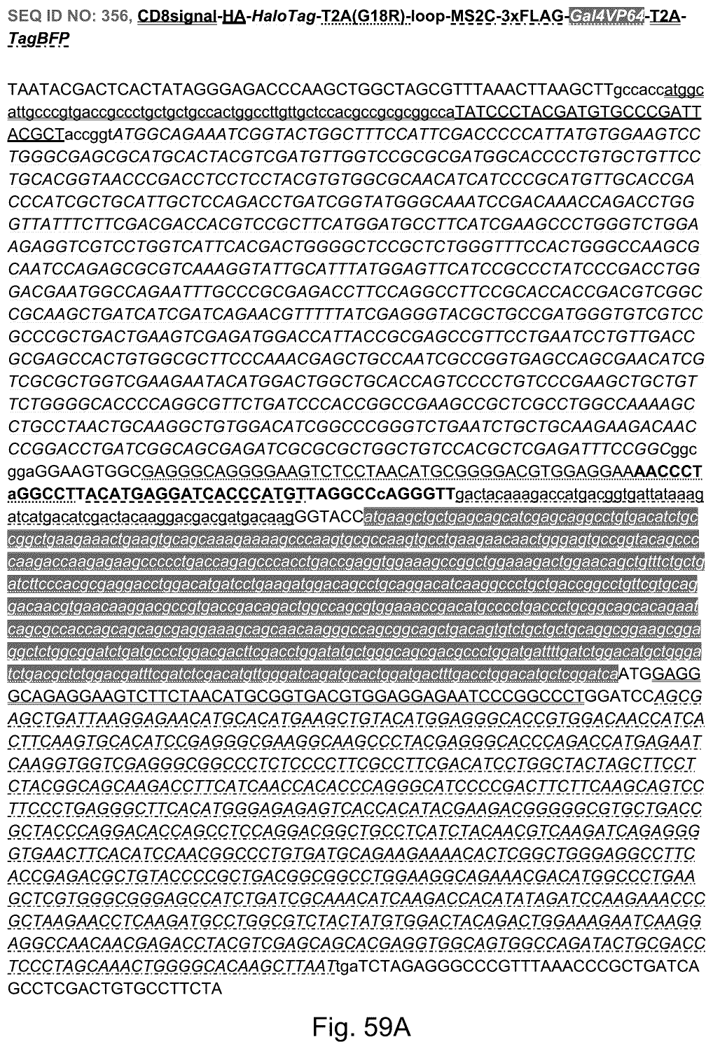

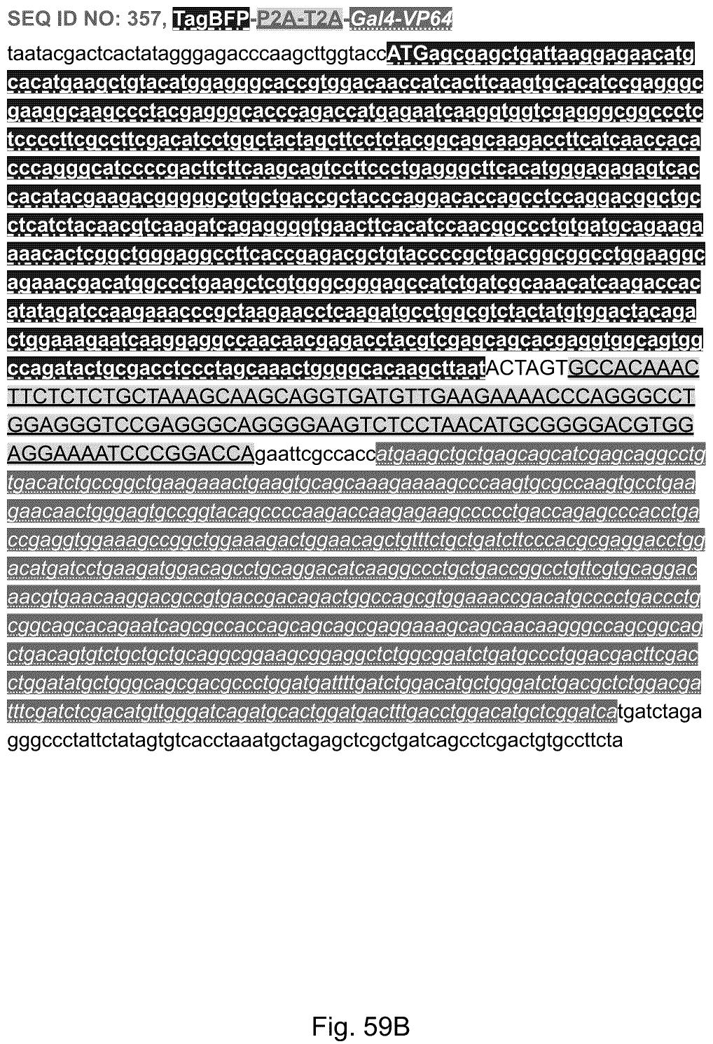

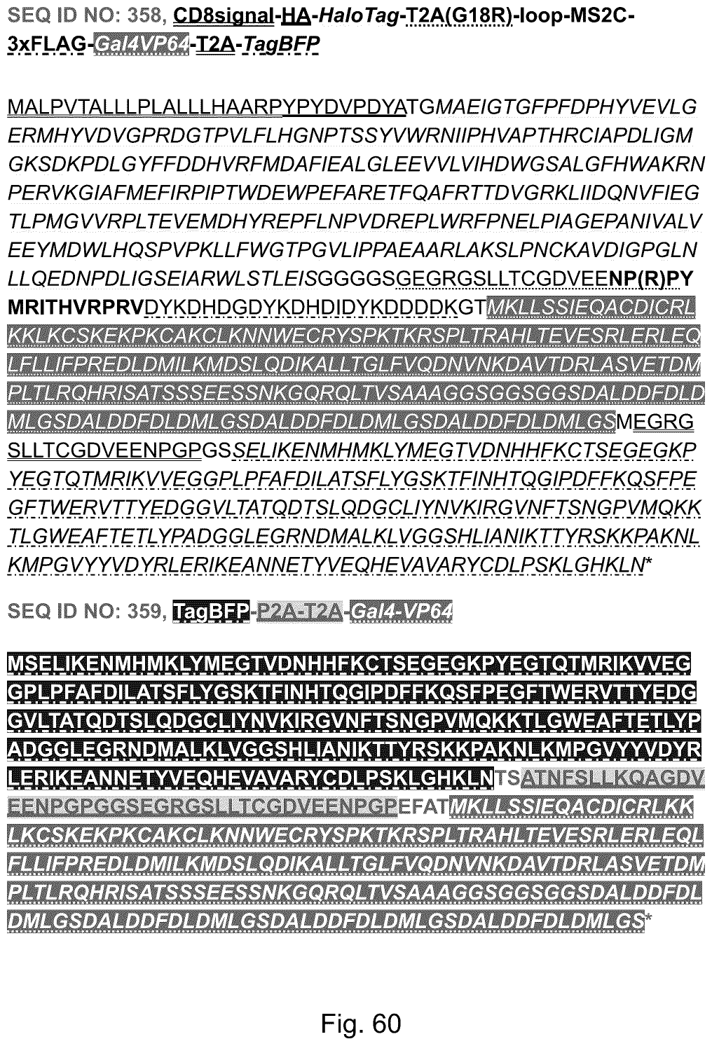

In some embodiments, the ds-SENSE region comprises a sense sequence as tested in any of FIGS. 29 or 37, as shown in any of FIGS. 41, 59, or 60, as shown in Table 15, or included in any of SEQ ID NO: 301, 356, or 357 or a nucleic acid sequence comprising at least 10 consecutive nucleotides selected from SEQ ID NO: 301, 356, or 357, or a nucleic acid sequence having at least 70%, or about 75%, or about 80%, or about 85%, or about 90%, or about 92%, or about 94% or about 96% or about 98%, or about 99% or 100% sequence identity to any of SEQ ID NO: 301, 356, or 357.

ii. Binding Motifs

Described herein are RNA molecules comprising at least one binding motif that can bind to an RNA binding domain (RBD) (e.g., of an iAD polypeptide as described further herein). In some embodiments, the RNA molecule comprises 1, 2, 3, 4, 5, 6, 7, 8, 9, 10, or more binding motifs. In some embodiments, the at least one binding motif is present in a double-stranded region of the RNA molecule.

In some embodiments, the double-stranded region (e.g., ds-STOP, ds-START, or ds-SENSE) region comprises at least one binding motif that binds to the RNA binding domain (RBD) selected from the group consisting of MS2, PP7, BoxB, and HIV TAR, as disclosed in Table 7.

In some embodiments, the double-stranded region (e.g., ds-STOP, ds-START, or ds-SENSE) region comprises at least one MS2 RBD binding motif comprising ACATGAGGATcACCCATGT (SEQ ID NO: 403) or a sequence at least 80%, or at least about 85%, or at least about 90% or at least about 95% sequence identity to SEQ ID NO: 130. It is contemplated herein that a sequence variant of MS2 RBD binding motif can be used, that maintains its binding function.

In some embodiments, the double-stranded region (e.g., ds-STOP, ds-START, or ds-SENSE) region comprises at least one PP7 RBD binding motif comprising ggagcagacgatatggcgtcgctcc (SEQ ID NO: 131) or a sequence at least 80%, or at least about 85%, or at least about 90% or at least about 95% sequence identity to SEQ ID NO: 131. It is contemplated herein that a sequence variant of PP7 RBD binding motif can be used, that maintains its binding function; for example, Lim and Peabody, “RNA recognition site of PP7 coat protein” Nucleic Acids Research 30(19):4138-44 (2002), the contents of which are incorporated herein by reference in its entirety, describes non-limiting examples of PP7 sequence variants,

In some embodiments, the double-stranded region (e.g., ds-STOP, ds-START, or ds-SENSE) region comprises at least one BoxB RBD binding motif comprising gggccctgaagaagggccc (SEQ ID NO: 132) or a sequence at least 80%, or at least about 85%, or at least about 90% or at least about 95% sequence identity to SEQ ID NO: 132. It is contemplated herein that a sequence variant of BoxB RBD binding motif can be used, that maintains its binding function.

In some embodiments, the double-stranded region (e.g., ds-STOP, ds-START, or ds-SENSE) region comprises at least one HIV Tar RBD binding motif comprising ggctcgtctgagctcattagctccgagcc (SEQ ID NO: 133) or a sequence at least 80%, or at least about 85%, or at least about 90% or at least about 95% sequence identity to SEQ ID NO: 133. It is contemplated herein that a sequence variant of HIV Tar RBD binding motif can be used, that maintains its binding function.

TABLE 7
Binding motif for RBD (BM)

| | | | Is bound | |
|---|---|---|---|---|
| | | | by RNA | |
| | | | Binding | SEQ ID |
| | | | Domain | NO: of |
| SEQ ID | Binding motif | | (RBD) of | the |
| NO: | for RBD (BM) | Sequence | the iAD | RBD |
| 403 | MS2 | ACATGAGGATcACCCATGT | MCP | 100 |
| 131 | PP7 | ggagcagacgatatggcgtcgctcc | PCP | 101 |
| 132 | BoxB | gggccctgaagaagggccc | λN | 102 |
| 133 | HIV TAR | ggctcgtctgagctcattagctccgagcc | HIV Tat | 103 |

In some embodiments, the Binding motif for RBD (BM) can be located after the stop codon sequence. In some embodiments, the Binding motif for RBD (BM) can be located before a stop codon sequence, e.g., a stop codon sequence disclosed in Table 6.

### B. Target Activation Construct (TAC)

In some embodiments, an exemplary Target Activation construct (TAC) is a synthetic RNA molecule, that comprises at least (i) a double stranded region, referred to herein as “ds-STOP” region, for example, but not limited to, a hairpin loop, where the ds-STOP region comprises, at least one stop codon; and a binding motif for RBD capable of being bound by an RNA-binding domain of the DD; and (ii) a second open reading frame, wherein the second open reading frame (2^{nd} ORF) is operatively linked to the double-stranded region. In some embodiments, the second ORF comprises a nucleic acid encoding a GOI of interest. The GOI is a nucleic acid transcript, can encode, for example but not limited to; a protein of interest to be expressed, mRNA, miRNA, antisense, and the like. In some embodiments, the TAC can comprise upstream of the ds-STOP region, a first open reading frame (1^{st} ORF). In some embodiments, the 1^{st} ORF comprises a nucleic acid sequence that encodes for a first polypeptide, and the second reading frame encodes for a second polypeptide. In some embodiments, the first open reading frame encodes for a first portion of a polypeptide, and the second reading frame encodes for a second portion of the polypeptide. In some embodiments, the first open reading frame comprises a nucleic acid sequence that encodes for the iADAR fusion protein as described herein.

In some embodiments, the second ORF, e.g., the nucleic acid sequence located 3′ of the ds-STOP region encodes an effector molecule or effector protein, as disclosed herein.

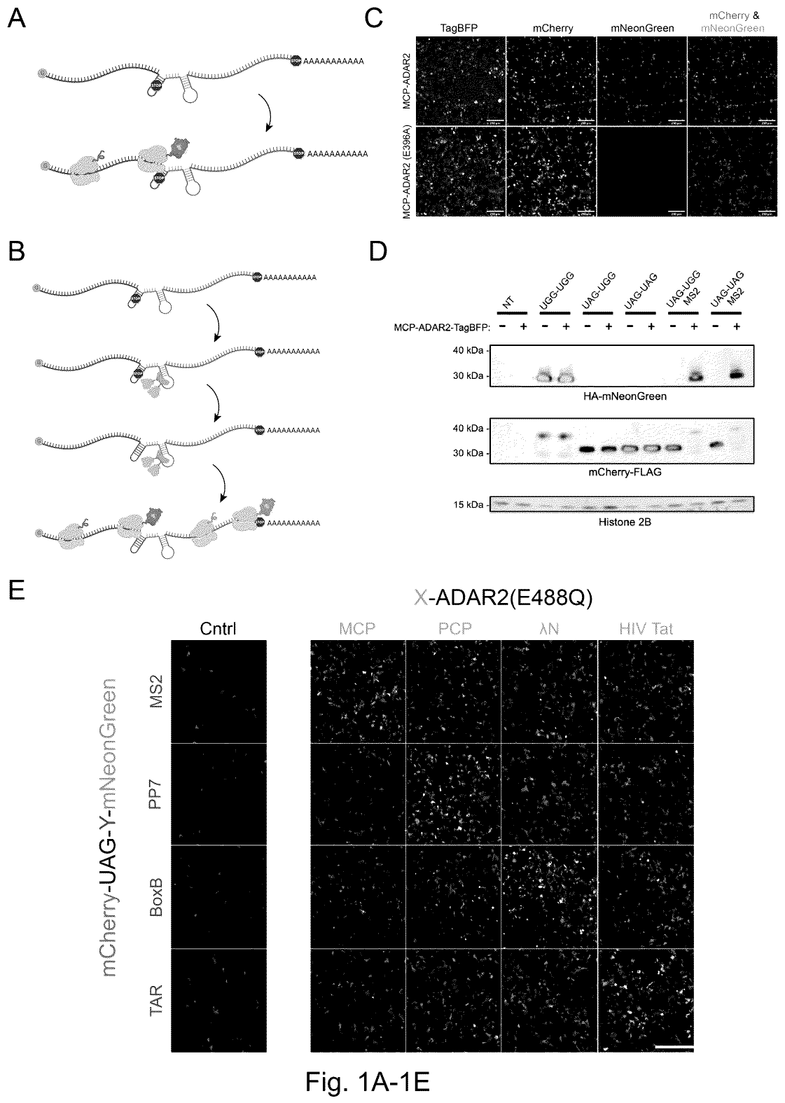

In some embodiments, the TAC comprises a synthetic RNA construct as exemplified by FIG. 1A-1B: an upstream coding region, a short hairpin that contains 1 or more stop codons, an RBD binding motif, and a downstream coding region.

### C. Target Inactivation Construct (TIC)

In some embodiments, an exemplary Target iNactivation construct (TIC) is a synthetic RNA molecule, that comprises, in the following order: (i) a first open reading frame that is operatively linked to the double-stranded STOP region, (ii) as least a ds-STOP region, as defined herein, for example, but not limited to, a hairpin loop, where the ds-STOP region comprises, at least one stop codon; and a binding motif for RBD capable of being bound by an RNA-binding domain of the DD; and (iii) a poly A region. In some embodiments, the first ORF comprises a nucleic acid encoding a GOI of interest. The GOI is a nucleic acid transcript, can encode, for example but not limited to; a protein of interest to be expressed, mRNA, miRNA, antisense, and the like. In some embodiments, in place of or in addition to the polyA region, the synthetic RNA molecule comprises a ribosome stalling sequence, which can lead to RNA degradation. Non-limiting examples of ribosome stalling sequences are known in the art, see e.g., Yip and Shao, “Detecting and Rescuing Stalled Ribosomes,” Trends in Biochemical Sciences, Volume 46, Issue 9, P731-743, September 2021, the contents of which are incorporated herein by reference in their entirety.

In some aspects of embodiments disclosed herein, an RNA molecule can comprise: (a) an open reading frame; (b) a ds-STOP region, comprising (i) at least one stop codon; and (ii) a binding motif for RBD capable of being bound by an RNA-binding domain; and (c) a poly-A tail.

In some aspects of embodiments disclosed herein, an RNA molecule (e.g., a TIC) can comprise: (a) a ds-START region, comprising (i) at least one start codon; and (ii) a binding motif for RBD capable of being bound by an RNA-binding domain; and (b) an open reading frame.

In some embodiments of any of the aspects, the RNA-binding domain comprises MCP, and the Binding motif for RBD (BM) comprises MS2. In some embodiments of any of the aspects, the RNA-binding domain comprises PCP, and the Binding motif for RBD (BM) comprises PP7. In some embodiments of any of the aspects, the RNA-binding domain comprises λN, and the Binding motif for RBD (BM) comprises BoxB. In some embodiments of any of the aspects, the RNA-binding domain comprises HIV Tat, and the Binding motif for RBD (BM) comprises TAR.

In some embodiments of any of the aspects, the double-stranded region of the RNA molecule comprises at least one hairpin. In some embodiments of any of the aspects, the double-stranded region of the RNA molecule comprises one hairpin comprising the at least one stop codon and the Binding motif for RBD (BM). In some embodiments of any of the aspects, the double-stranded region of the RNA molecule comprises a first hairpin comprising the at least one stop codon and a second hairpin comprising the Binding motif for RBD (BM).

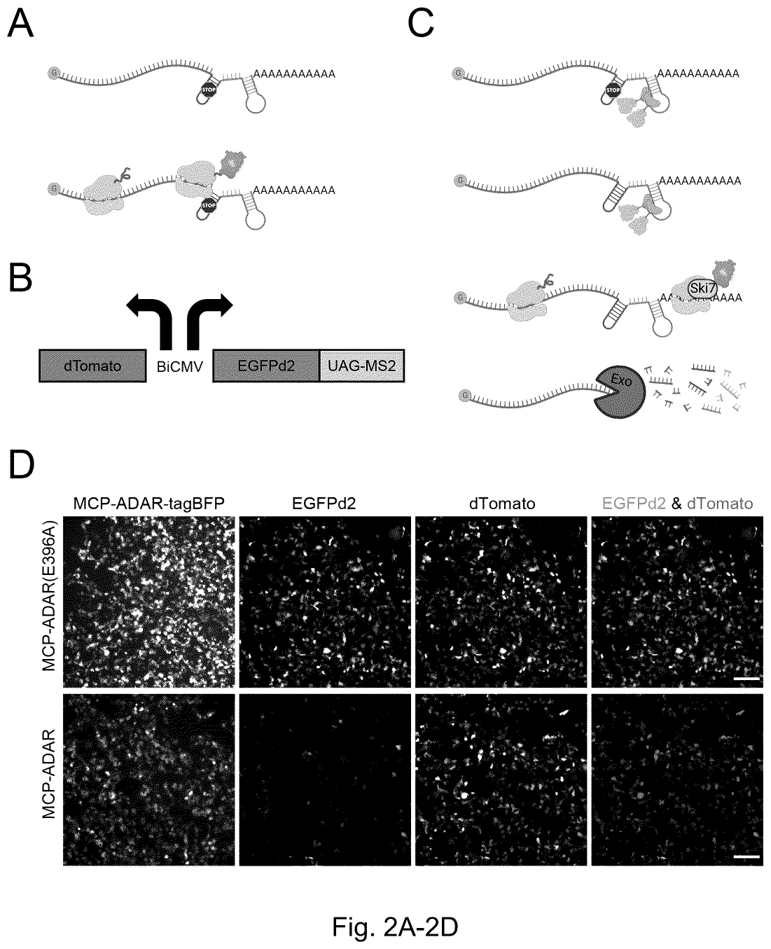

In some embodiments, the TiC comprises a synthetic RNA construct as exemplified by FIG. 2A-2C: an upstream coding region, a short hairpin that contains 1 or more stop codons, an RBD binding motif, and a polyA tail or ribosome stalling sequence.

### D. Synthetic Construct Comprising Both a TAC and TIC

In some embodiments, a synthetic construct comprises both a TAC and TIC. Such a construct enables switching of the translation of one GOI to another GOI. For exemplary purposes only, in one embodiment, a synthetic construct comprises, in the following order, a first ORF, a first ds-STOP region, a polyA sequence, a second ds-STOP region, and a second ORF. In such an embodiment, when the inducer is absent, the iADAR-OFF enables the translation of the first ORF only (1-ORF expressed only). When the inducer is present, e.g., the iADAR is in ON state (iADAR-ON) the first ds-STOP is edited and therefore the polyA sequence is translation and results in the transcript for the first ORF undergoing mRNA decay (e.g., 1^{st}-ORF decay/OFF) and the second ds-STOP is edited therefore enabling translation of the second ORF (e.g., 2^{nd}-ORF is expressed). In some embodiments, such a transcript comprising a TAC and TIN can comprise an IRES located between the first and second ds-STOP regions. In some embodiments, the first ORF encodes an effector molecule, and the second ORF encodes a second effector molecule, where the second effector molecule is a second or alternative version of the first effector molecule. Stated differently, using such a system, in the presence of the inducer, one can easily switch the expression from the first ORF (e.g., transcript A) to the second ORF (e.g., transcript B).

### E. Effector Protein.

In some embodiments, the GOI is a nucleic acid transcript, which can encode, for example but not limited to: a protein of interest to be expressed as an effector protein, and the like. Effector molecules are well known in the art and can include, but are not limited to, antibodies, enzymes, chimeric antigen receptors (CARs). In some embodiments, the effector protein comprises an antigen-binding domain for a cancer antigen. In some embodiments, the effector protein comprises an antigen-binding domain for a microbial antigen.

In some embodiments the effector protein comprises a detectable marker or a reporter molecule, including but not limited to a fluorescent protein or a detectable tag (e.g., c-Myc, HA, VSV-G, HSV, FLAG, V5, HIS, or biotin). In some embodiments of any of the aspects, an effector protein as described herein, especially those that are administered to a subject or those that are part of a pharmaceutical composition, do not comprise detectable markers that are immunogenic. In some embodiments of any of the aspects, an effector protein as described herein do not comprise GFP, mCherry, HA1, or any other immunogenic markers. In some embodiments of any of the aspects, an effector protein described herein that comprises a detectable marker can have the detectable marker removed at a later time, e.g., a removable (e.g., cleavable) detectable marker. In some embodiments of any of the aspects, an effector protein described herein that comprises a detectable marker can have the detectable marker replaced with a different detectable marker, as known in the art or described herein, e.g., a replaceable (e.g., interchangeable) detectable marker.

### III. Systems

Another aspect of the technology relates to systems and cells comprising an iADAR and a nucleic effector construct, e.g., an activation construct or inactivation construct as disclosed herein.

In certain embodiments, the iADAR is naturally or endogenously present in the host cell, for example, naturally or endogenously present in the eukaryotic cell. In some embodiments, the ADAR is endogenously expressed by the host cell. In certain embodiments, the iADAR is exogenous to the host cell. In some embodiments, the iADAR is encoded by a nucleic acid (e.g., DNA or RNA). In some embodiments, the method comprises introducing the iADAR or a construct encoding the iADAR into the host cell. In some embodiments, the method does not comprise introducing any protein into the host cell. In certain embodiments, the iADAR is iADAR1 and/or iADAR2. In some embodiments, the iADAR is one or more iADARs selected from the group consisting of hiADAR1, hiADAR2, murine iADAR1 and murine iADAR2.

In one aspect described herein is a system for modulating RNA translation comprising: (a) a fusion protein comprising an RNA-binding domain linked to a deaminase domain of an adenosine deaminase; and (b) an RNA molecule comprising: (i) an open reading frame; (ii) a double-stranded region comprising: (A) at least one target codon (e.g., stop, start, non-start, or sense codon); and (B) a binding motif for RBD capable of being bound by the RNA-binding domain of the fusion protein; and (iii) a poly-A tail.

In one aspect described herein is a system for modulating RNA translation comprising: (a) a fusion protein comprising: (i) an RNA-binding domain; (ii) a first portion of a deaminase domain (DD) of an adenosine deaminase; (iii) a first member of a binding pair associated with the first portion of the DD; (iv) a second portion of the DD; and (v) a second member of a binding pair associated with the second portion of the DD; and (b) an RNA molecule as described herein.

In one aspect described herein is a system for modulating RNA translation comprising: (a) a fusion protein comprising: (i) an RNA-binding domain; (ii) a first portion of a deaminase domain (DD) of an adenosine deaminase; (iii) a first member of a binding pair associated with the first portion of the DD; (iv) a second portion of the DD; and (v) a second member of a binding pair associated with the second portion of the DD; and (b) an RNA molecule comprising: (i) a first open reading frame; (ii) a double-stranded region comprising: (A) at least one target codon (stop, start, non-start, or sense codon); and (B) a binding motif for RBD capable of being bound by the RNA-binding domain of the fusion protein; and (iii) a second open reading frame.

In one aspect described herein is a system for modulating RNA translation comprising: (a) a fusion protein comprising: (i) an RNA-binding domain; (ii) a first portion of a deaminase domain (DD) of an adenosine deaminase; (iii) a first member of a binding pair associated with the first portion of the DD; (iv) a second portion of the DD; and (v) a second member of a binding pair associated with the second portion of the DD; and (b) an RNA molecule comprising: (i) an open reading frame; (ii) a double-stranded region comprising: (A) at least one target codon (stop, start, non-start, or sense codon); and (B) a binding motif for RBD capable of being bound by the RNA-binding domain of the fusion protein; and (iii) a poly-A tail.

In one aspect described herein is a system for modulating RNA translation comprising: (a) a fusion protein comprising: (i) an RNA-binding domain; (ii) a first portion of a deaminase domain (DD) of an adenosine deaminase; (iii) a first member of a binding pair associated with the first portion of the DD; (iv) a second portion of the DD; (v) a cleavable linker; and (vi) a second member of a binding pair associated with the cleavable linker; and (b) an RNA molecule as described herein.

In one aspect described herein is a system for modulating RNA translation comprising: (a) a fusion protein comprising: (i) an RNA-binding domain; (ii) a first portion of a deaminase domain (DD) of an adenosine deaminase; (iii) a first member of a binding pair associated with the first portion of the DD; (iv) a second portion of the DD; (v) a cleavable linker; and (vi) a second member of a binding pair associated with the cleavable linker; and (b) an RNA molecule comprising: (i) a first open reading frame; (ii) a double-stranded region comprising: (A) at least one target codon (stop, start, non-start, or sense codon); and (B) a binding motif for RBD capable of being bound by the RNA-binding domain of the fusion protein; and (iii) a second open reading frame.

In one aspect described herein is a system for modulating RNA translation comprising: (a) a fusion protein comprising: (i) an RNA-binding domain; (ii) a first portion of a deaminase domain (DD) of an adenosine deaminase; (iii) a first member of a binding pair associated with the first portion of the DD; (iv) a second portion of the DD; (v) a cleavable linker; and (vi) a second member of a binding pair associated with the cleavable linker; and (b) an RNA molecule comprising: (i) an open reading frame; (ii) a double-stranded region comprising: (A) at least one target codon (stop, start, non-start, or sense codon); and (B) a binding motif for RBD capable of being bound by the RNA-binding domain of the fusion protein; and (iii) a poly-A tail.

In some embodiments, the deaminase domain is capable of converting the at least one stop codon into at least one non-stop codon. In some embodiments, the first reading frame is translated when the at least one stop codon is present in the double-stranded region of the RNA molecule. In some embodiments, the RNA molecule is degraded when the at least one stop codon is converted into the at least one non-stop codon.

In some embodiments, the deaminase domain is capable of converting the at least one start codon into at least one non-start codon. In some embodiments, the reading frame is translated when the at least one start codon is present in the double-stranded region of the RNA molecule. In some embodiments, the RNA molecule is not translated when the at least one start codon is converted into the at least one non-start codon.

In some embodiments, the deaminase domain is capable of converting the at least one non-start codon into at least one start codon. In some embodiments, the RNA molecule is translated when the at least one non-start codon is converted into the at least one start codon. In some embodiments, the reading frame is not translated when the at least one non-start codon is present in the double-stranded region of the RNA molecule.

In some embodiments, the deaminase domain is capable of converting the at least one sense codon into at least one mutated sense codon. In some embodiments, the structure and/or function of the RNA and/or encoded polypeptide is altered when the at least one sense codon is converted into the at least one mutated sense codon.

In some embodiments, the system further comprising an inducer of the first and second binding pairs. Depending on the affinity binding pair of the iADAR, inducers can be, but are not limited to, small molecules, proteases, light-inducible control, sound inducible control, cell cycle dependent, ultrasound or other wavelength dependent triggers, chemically cleavable linkers, heat-activated triggers, antibodies, endogenous triggers, disease triggers, external triggers and cell-specific marker triggers, and the like. Non-limiting examples of small molecule inducers include A-1331852, ABT-737, and S63845 as described further herein. Non-limiting examples of chemically cleavable linkers include click-release based chemistry, see e.g., van Onzen et al., “Bioorthogonal Tetrazine Carbamate Cleavage by Highly Reactive trans-Cyclooctene, J. Am. Chem. Soc. 2020, 142, 25, 10955-10963, the content of which is incorporated herein by reference in its entirety. Non-limiting examples of ultrasound dependent triggers can use chemical means or gas vesicles, see e.g., Berkowski et al., “Ultrasound-Induced Site-Specific Cleavage of Azo-Functionalized Poly(ethylene glycol),” Macromolecules 2005, 38, 22, 8975-8978; Farhadi et al., “Ultrasound Imaging of Gene Expression in Mammalian Cells,” Science. 2019 Sep. 27; 365(6460): 1469-1475; the contents of each of which are incorporated herein by reference in their entireties.

In embodiments using a repressible protease and its cognate protease domain as the binding pair of the iADAR, the inducer can be a protease inhibitor, e.g., selected from grazoprevir, danoprevir, simeprevir, asunaprevir, ciluprevir, boceprevir, sovaprevir, paritaprevir, ombitasvir, paritaprevir, ritonavir, dasabuvir, and telaprevir or Table 9.

In some embodiments, the first and second members of the binding pair of the fusion protein bind to each other in the absence of an inducer of the first and second binding pairs. Binding of the first and second members of the binding pair can reduce or prevent at least one of the following: the formation of the inositol hexaphosphate (IP6) binding pocket of first and second portions of the deaminase domain; deaminase activity of the first and second portions of the deaminase domain; conversion by the deaminase domain of the at least one target codon of the RNA molecule into at least one inosine-comprising codon; conversion by the deaminase domain of the at least one stop codon of the RNA molecule into at least one non-stop codon; conversion by the deaminase domain of the at least one start codon of the RNA molecule into at least one non-start codon; conversion by the deaminase domain of the at least one non-start codon of the RNA molecule into at least one start codon; conversion by the deaminase domain of the at least one sense codon of the RNA molecule encoding for a first amino acid into at least one mutated sense codon encoding for a second amino acid; translation of a reading frame (e.g., the second reading frame of the RNA molecule); and/or degradation of the RNA molecule.

In some embodiments, the first and second members of the binding pair of the fusion protein do not bind to each other in the presence of an inducer of the first and second binding pairs, allowing for or increasing at least one of the following outcomes: the formation of the inositol hexaphosphate (IP6) binding pocket of first and second portions of the deaminase domain; deaminase activity of the first and second portions of the deaminase domain; conversion by the deaminase domain of the at least one target codon of the RNA molecule into at least one inosine-comprising codon; conversion by the deaminase domain of the at least one stop codon of the RNA molecule into at least one non-stop codon; conversion by the deaminase domain of the at least one start codon of the RNA molecule into at least one non-start codon; conversion by the deaminase domain of the at least one non-start codon of the RNA molecule into at least one start codon; conversion by the deaminase domain of the at least one sense codon of the RNA molecule encoding for a first amino acid into at least one mutated sense codon encoding for a second amino acid; translation of a reading frame (e.g., the second reading frame) of the RNA molecule; and/or degradation of the RNA molecule.

In systems comprising a cleavable linker in the iADAR, the system can further comprise a cleavage inducer. Depending on the cleavable linker used in the iADAR, the cleavage inducer can be light, sound, ultrasound, chemical, heat, endogenous triggers, disease triggers, external triggers and cell-specific marker triggers, and the like.

In some embodiments, the cleavable linker is not cleaved in the absence of a cleavage inducer. Lack of cleavage of the cleavable linker can reduce or prevent at least one of the following outcomes: the formation of the inositol hexaphosphate (IP6) binding pocket of first and second portions of the deaminase domain; deaminase activity of the first and second portions of the deaminase domain; conversion by the deaminase domain of the at least one target codon of the RNA molecule into at least one inosine-comprising codon; conversion by the deaminase domain of the at least one stop codon of the RNA molecule into at least one non-stop codon; conversion by the deaminase domain of the at least one start codon of the RNA molecule into at least one non-start codon; conversion by the deaminase domain of the at least one non-start codon of the RNA molecule into at least one start codon; conversion by the deaminase domain of the at least one sense codon of the RNA molecule encoding for a first amino acid into at least one mutated sense codon encoding for a second amino acid; translation of a reading frame (e.g., the second reading frame) of the RNA molecule; and/or degradation of the RNA molecule.

In some embodiments, the cleavable linker is cleaved in the presence of a cleavage inducer, which can allow or increase one of the following outcomes: the formation of the inositol hexaphosphate (IP6) binding pocket of first and second portions of the deaminase domain; deaminase activity of the first and second portions of the deaminase domain; conversion by the deaminase domain of the at least one target codon of the RNA molecule into at least one inosine-comprising codon; conversion by the deaminase domain of the at least one stop codon of the RNA molecule into at least one non-stop codon; conversion by the deaminase domain of the at least one start codon of the RNA molecule into at least one non-start codon; conversion by the deaminase domain of the at least one non-start codon of the RNA molecule into at least one start codon; conversion by the deaminase domain of the at least one sense codon of the RNA molecule encoding for a first amino acid into at least one mutated sense codon encoding for a second amino acid; translation of a reading frame (e.g., the second reading frame) of the RNA molecule; and/or degradation of the RNA molecule.

In some embodiments, the at least one stop codon of the synthetic RNA molecule comprises UAG. In some embodiments, the at least one non-stop codon of the synthetic RNA molecule comprises at least one tryptophan codon. In some embodiments, the at least one tryptophan codon of the synthetic RNA molecule comprises UGG.

In some embodiments, the RNA-binding domain comprises MCP, and the Binding motif for RBD (BM) comprises MS2. In some embodiments, the RNA-binding domain comprises PCP, and the Binding motif for RBD (BM) comprises PP7. In some embodiments, the RNA-binding domain comprises λN, and the Binding motif for RBD (BM) comprises BoxB. In some embodiments, the RNA-binding domain comprises HIV Tat, and the Binding motif for RBD (BM) comprises TAR.

In some embodiments, the double-stranded region of the RNA molecule comprises at least one hairpin. In some embodiments, the double-stranded region of the RNA molecule comprises one hairpin comprising the at least one target codon (stop, start, non-start, or sense codon) and the Binding motif for RBD (BM). In some embodiments, the double-stranded region of the RNA molecule comprises a first hairpin comprising the at least one target codon (stop, start, non-start, or sense codon) and a second hairpin comprising the Binding motif for RBD (BM).

### IV. Nucleic Acids

Described herein are various nucleic acids. In one aspect, described herein is a nucleic acid encoding a fusion protein (e.g., iAD, iADAR) as described herein. In one aspect, described herein is a nucleic acid encoding a synthetic RNA molecule as described herein. In one aspect, described herein is a nucleic acid encoding a fusion protein and a synthetic RNA molecule as described herein.

In some embodiments, the nucleic acid encoding the fusion protein and the nucleic acid encoding the RNA molecule are operably linked to a single promoter. In some embodiments, the nucleic acid encoding the fusion protein and the nucleic acid encoding the RNA molecule are each operably linked to a separate promoter. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule. In some embodiments, the nucleic acid encoding the fusion protein is linked to and 5′ of the nucleic acid encoding the RNA molecule. In some embodiments, the nucleic acid encoding the fusion protein is linked to and 3′ of the nucleic acid encoding the RNA molecule.

In some embodiments, the nucleic acid comprises DNA. In some embodiments, the nucleic acid comprises RNA. In some embodiments, the nucleic acid comprises RNA and DNA.

In some embodiments, the nucleic acid is one of SEQ ID NOs: 38-73, 95, 99, 173, 177, 181, 185, 189, 193, 197, 199, 201, 203, 205, or 207, or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 38-73, 95, 99, 173, 177, 181, 185, 189, 193, 197, 199, 201, 203, 205, or 207, that maintains the same function, or a codon-optimized version thereof.

In some embodiments of any of the aspects, a nucleic acid (e.g. DNA, or RNA transcript disclosed herein) is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of nucleic acid compounds useful in the embodiments described herein include, but are not limited to nucleic acids containing modified backbones or no natural internucleoside linkages. nucleic acids having modified backbones include, among others, those that do not have a phosphorus atom in the backbone. For the purposes of this specification, and as sometimes referenced in the art, modified nucleic acids that do not have a phosphorus atom in their internucleoside backbone can also be considered to be oligonucleosides. In some embodiments of any of the aspects, the modified nucleic acid will have a phosphorus atom in its internucleoside backbone.

Modified nucleic acid backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified nucleic acid backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH-CH2-, —CH2-N(CH3)-O-CH2-[known as a methylene (methylimino) or MMI backbone], —CH2-O—N(CH3)-CH2-, —CH2-N(CH3)-N(CH3)-CH2- and —N(CH3)-CH2-CH2-[wherein the native phosphodiester backbone is represented as —O—P—O—CH2-].

In other nucleic acid mimetics, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The nucleic acid can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol. Canc. Ther. 6(3):833-843; Grunweller, A. et al., (2003) Nucleic Acids Research 31(12):3185-3193).

Modified nucleic acids can also contain one or more substituted sugar moieties. The nucleic acids described herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO]mCH3, O(CH2)nOCH3, O(CH2)nNH2, O(CH2) nCH3, O(CH2)nONH2, and O(CH2)nON[(CH2)nCH3)]2, where n and m are from 1 to about 10. In some embodiments of any of the aspects, nucleic acids include one of the following at the 2′ position: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleic acid, or a group for improving the pharmacodynamic properties of a nucleic acid, and other substituents having similar properties. In some embodiments of any of the aspects, the modification includes a 2′ methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2—O—CH2-N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the nucleic acid, particularly the 3′ position of the sugar on the 3′ terminal nucleotide or in 2′-5′ linked dsRNAs and the 5′ position of 5′ terminal nucleotide. Nucleic acids may also have sugar mimetics such as cyclobutyl moieties in place of the pentofuranosyl sugar.

A nucleic acid can also include nucleobase (often referred to in the art simply as “base”) modifications or substitutions. As used herein, “unmodified” or “natural” or “canonical” nucleobases include the purine bases adenine (A) and guanine (G), and the pyrimidine bases thymine (T), cytosine (C) and uracil (U). Modified or “non-canonical” nucleobases can include other synthetic and natural nucleobases including but not limited to as inosine, isocytosine, isoguanine, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications. In some embodiments of any of the aspects, modified nucleobases can include d5SICS and dNAM, which are a non-limiting example of unnatural nucleobases that can be used separately or together as base pairs (see e.g., Leconte et. al. J. Am. Chem. Soc. 2008, 130, 7, 2336-2343; Malyshev et. al. PNAS. 2012. 109 (30) 12005-12010). In some embodiments of any of the aspects, the nucleic acid comprises any modified nucleobases known in the art, i.e., any nucleobase that is modified from an unmodified and/or natural nucleobase.

The preparation of the modified nucleic acids, backbones, and nucleobases described above are well known in the art.

Another modification of a nucleic acid featured in the invention involves chemically linking to the nucleic acid to one or more ligands, moieties or conjugates that enhance the activity, cellular distribution, pharmacokinetic properties, or cellular uptake of the nucleic acid. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

### V. Vectors

In some embodiments, one or more of the nucleic acids encoding a synthetic STOP region that is operatively linked to a GOI or transcript of interest as disclosed herein is expressed in a recombinant expression vector or plasmid. In some embodiments, a synthetic target activation construct (TAC) or target inactivation construct (TIC), or both, as disclosed herein, are expressed in a recombinant expression vector or plasmid. In some embodiments, a TIC or TAC can comprise one or more nucleic acids encoding an iAD, e.g., iADAR as disclosed herein is expressed in a recombinant expression vector or plasmid. In some embodiments, a vector (e.g., a lentivirus) express (A) iADAR and TAC RNA, (B) iADAR and TIC, or (C) at least one iADAR, TAC RNA, and TIC RNA, for example one iADAR that acts on the TAC RNA and another iADAR that acts on the TIC RNA. In some embodiments, one or more of the nucleic acids encoding an iAD, e.g., iADAR can be as disclosed herein is expressed in a recombinant expression vector or plasmid. In some embodiments, the TIC or TAC RNA (e.g., comprising a GOI) is delivered by lentivirus or non-viral constructs, e.g., closed ended DNA (ceDNA), etc.

As used herein, the term “vector” refers to a polynucleotide sequence suitable for transferring transgenes into a host cell. The term “vector” includes plasmids, mini-chromosomes, phage, naked DNA and the like. See, for example, U.S. Pat. Nos. 4,980,285; 5,631,150; 5,707,828; 5,759,828; 5,888,783 and, 5,919,670, and, Sambrook et al, Molecular Cloning: A Laboratory Manual, 2nd Ed., Cold Spring Harbor Press (1989). One type of vector is a “plasmid,” which refers to a circular double stranded DNA loop into which additional DNA segments are ligated. Another type of vector is a viral vector, wherein additional DNA segments are ligated into the viral genome. Certain vectors are capable of autonomous replication in a host cell into which they are introduced (e.g., bacterial vectors having a bacterial origin of replication and episomal mammalian vectors). Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” is used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

A cloning vector is one which is able to replicate autonomously or integrated in the genome in a host cell, and which is further characterized by one or more endonuclease restriction sites at which the vector may be cut in a determinable fashion and into which a desired DNA sequence can be ligated such that the new recombinant vector retains its ability to replicate in the host cell. In the case of plasmids, replication of the desired sequence can occur many times as the plasmid increases in copy number within the host cell such as a host bacterium or just a single time per host before the host reproduces by mitosis. In the case of phage, replication can occur actively during a lytic phase or passively during a lysogenic phase.

An expression vector is one into which a desired DNA sequence can be inserted by restriction and ligation such that it is operably joined to regulatory sequences and can be expressed as an RNA transcript. Vectors can further contain one or more marker sequences suitable for use in the identification of cells which have or have not been transformed or transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). In certain embodiments, the vectors used herein are capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the polypeptides described herein is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression can vary between species or cell types, but in general can include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences can also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell.

In some embodiments, one or more of the recombinantly expressed gene can be integrated into the genome of the cell.

A nucleic acid molecule that encodes the enzyme of the claimed invention can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention also may be accomplished by integrating the nucleic acid molecule into the genome.

### VI. Cells and Compositions

In one aspect, described herein is a cell comprising at least one fusion protein (e.g., iAD, iADAR) as described herein. In one aspect, described herein is a cell comprising at least one synthetic RNA molecule (e.g., TIC, TAC) as described herein. In one aspect, described herein is a cell comprising at least one nucleic acid as described herein. In one aspect, described herein is a cell comprising at least one vector as described herein. In one aspect, described herein is a cell comprising at least one system (e.g., iADAR and synthetic TIC or TAC RNA) as described herein.

In some embodiments, the cell is selected from the group consisting of a fibroblast, a hematopoietic cell, a neuron, a pancreatic cell, a muscle cell, a bone cell, a hepatocyte, a pancreatic cell, an epithelial cell, an endothelial cell, a cardiomyocyte, an immune cell (e.g., a T cell, a B cell), a liver cell, an osteocyte, and the like.

In one aspect, described herein is a composition comprising at least one fusion protein (e.g., iAD, iADAR) as described herein. In one aspect, described herein is a composition comprising at least one synthetic RNA molecule (e.g., TIC, TAC) as described herein. In one aspect, described herein is a composition comprising at least one nucleic acid as described herein. In one aspect, described herein is a composition comprising at least one vector as described herein. In one aspect, described herein is a composition comprising at least one system (e.g., iADAR and synthetic TIC or TAC RNA) as described herein. In one aspect, described herein is a composition comprising at least one cell as described herein. In some embodiments, the composition further comprises at least one inducer of the first and second binding pairs. In some embodiments, the composition further comprises at least one cleavage inducer. The composition can be in the form of a liquid, gel solid, powder, and the like.

In one aspect, described herein is a pharmaceutical composition comprising at least one a pharmaceutically compatible carrier at least one fusion protein (e.g., iAD, iADAR) as described herein. In one aspect, described herein is a pharmaceutical composition comprising at least one a pharmaceutically compatible carrier at least one synthetic RNA molecule (e.g., TIC, TAC) as described herein. In one aspect, described herein is a pharmaceutical composition comprising at least one a pharmaceutically compatible carrier at least one nucleic acid as described herein. In one aspect, described herein is a pharmaceutical composition comprising at least one a pharmaceutically compatible carrier at least one vector as described herein. In one aspect, described herein is a pharmaceutical composition comprising at least one a pharmaceutically compatible carrier at least one system (e.g., iADAR and synthetic TIC or TAC RNA) as described herein. In one aspect, described herein is a pharmaceutical composition comprising at least one a pharmaceutically compatible carrier at least one cell as described herein. In some embodiments, the composition further comprises at least one inducer of the first and second binding pairs. In some embodiments, the composition further comprises at least one cleavage inducer. The composition can be in the form of a liquid, gel solid, powder, and the like.

In some embodiments, the technology described herein relates to a pharmaceutical composition comprising at least one of (a) fusion protein, (b) RNA molecule, (c) nucleic acid, (d) vector, (e) system, (f) cell, and/or (g) composition, as described herein, and optionally a pharmaceutically acceptable carrier. In some embodiments, the active ingredients of the pharmaceutical composition comprise (a) fusion protein, (b) RNA molecule, (c) nucleic acid, (d) vector, (e) system, (f) cell, and/or (g) composition as described herein. In some embodiments, the active ingredients of the pharmaceutical composition consist essentially of (a) fusion protein, (b) RNA molecule, (c) nucleic acid, (d) vector, (e) system, (f) cell, and/or (g) composition as described herein. In some embodiments, the active ingredients of the pharmaceutical composition consist of (a) fusion protein, (b) RNA molecule, (c) nucleic acid, (d) vector, (e) system, (f) cell, and/or (g) composition as described herein.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (24) C₂-C₁₂ alcohols; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. (a) fusion protein, (b) RNA molecule, (c) nucleic acid, (d) vector, (e) system, (f) cell, and/or (g) composition as described herein.

In some embodiments, the pharmaceutical composition comprising at least one (a) fusion protein, (b) RNA molecule, (c) nucleic acid, (d) vector, (e) system, (f) cell, and/or (g) composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Pharmaceutical compositions comprising at least one (a) fusion protein, (b) RNA molecule, (c) nucleic acid, (d) vector, (e) system, (f) cell, and/or (g) composition as described herein can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia PA. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the pharmaceutical composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropyl methylcellulose, other polymer matrices, gels, permeable membranes, osmotic systems (such as OROS® (Alza Corporation, Mountain View, Calif. USA)), or a combination thereof to provide the desired release profile in varying proportions.

In some embodiments of any of the aspects, the at least one (a) fusion protein, (b) RNA molecule, (c) nucleic acid, (d) vector, (e) system, (f) cell, and/or (g) composition as described herein described herein is administered as a monotherapy, e.g., another treatment for the disease or disorder is not administered to the subject.

In some embodiments of any of the aspects, the methods described herein can further comprise administering a second agent and/or treatment to the subject, e.g. as part of a combinatorial therapy. Non-limiting examples of a second agent and/or treatment can include a cancer therapy selected from the group consisting of: radiation therapy, surgery, gemcitabine, cisplatin, paclitaxel, carboplatin, bortezomib, AMG479, vorinostat, rituximab, temozolomide, rapamycin, ABT-737, PI-103; alkylating agents such as thiotepa and CYTOXAN® cyclophosphamide; alkyl sulfonates such as busulfan, improsulfan and piposulfan; aziridines such as benzodopa, carboquone, meturedopa, and uredopa; ethylenimines and methylmelamines including altretamine, triethylenemelamine, trietylenephosphoramide, triethylenethiophosphoramide and trimethylol melamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomycins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In addition, the methods of treatment can further include the use of radiation or radiation therapy. Further, the methods of treatment can further include the use of surgical treatments.

The methods described herein can further comprise administering a second agent and/or treatment to the subject, e.g. as part of a combinatorial therapy. By way of non-limiting example, if a subject is to be treated for pain or inflammation according to the methods described herein, the subject can also be administered a second agent and/or treatment known to be beneficial for subjects suffering from pain or inflammation. Examples of such agents and/or treatments include, but are not limited to, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and the like.

In certain embodiments, an effective dose of a composition comprising at least one (a) fusion protein, (b) RNA molecule, (c) nucleic acid, (d) vector, (e) system, and/or (f) cell as described herein can be administered to a patient once. In certain embodiments, an effective dose of a composition comprising at least one (a) fusion protein, (b) RNA molecule, (c) nucleic acid, (d) vector, (e) system, and/or (f) cell as described herein can be administered to a patient repeatedly. For systemic administration, subjects can be administered a therapeutic amount of a composition comprising at least one (a) fusion protein, (b) RNA molecule, (c) nucleic acid, (d) vector, (e) system, and/or (f) cell as described herein, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g., by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the (a) fusion protein, (b) RNA molecule, (c) nucleic acid, (d) vector, (e) system, and/or (f) cell as described herein. The desired dose or amount can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition as described herein can be administered over a period of time, such as over a 5 minute, 10 minute, 15 minute, 20 minute, or 25 minute period.

The dosage ranges for the administration of a composition as described herein, according to the methods described herein depend upon, for example, the form of the composition, its potency, and the extent to which symptoms, markers, or indicators of a condition described herein are desired to be reduced, for example the percentage reduction desired for the disease or disorder or the extent to which, for example, immune reactions, are desired to be induced. The dosage should not be so large as to cause adverse side effects, such as autoimmunity. Generally, the dosage will vary with the age, condition, and sex of the patient and can be determined by one of skill in the art. The dosage can also be adjusted by the individual physician in the event of any complication.

The efficacy of a composition as described herein in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of cancer or infectious disease. When using an experimental animal model, efficacy of treatment is evidenced when a statistically significant change in a marker is observed.

### VI. Uses

In certain embodiments, the method using the iADAR can be used for editing on a target RNA to generate point mutation and/or misfolding of the protein encoded by the target RNA, and/or generating an early stop codon, an aberrant splice site, and/or an alternative splice site in the target RNA.

In certain embodiments, the iADAR-ON results in the deamination of a target (e.g., the target A) in the target RNA and results in a missense mutation, an early stop codon, aberrant splicing, or alternative splicing in the target RNA.

In certain embodiments, the iADAR-ON results in the deamination of a target (e.g., the target A) in the target RNA and results in deactivating or eliminating a STOP codon.

In some embodiments, the target RNA encodes a protein, and the deamination of a target (e.g., the target A) in the target RNA results in a point mutation, truncation, elongation and/or misfolding of the protein. In some embodiments, the iADAR-ON results in the deamination of a target (e.g., the target A) in the target RNA, and results in reversal of a missense mutation, an early stop codon, aberrant splicing, or alternative splicing in the target RNA. In some embodiments, wherein the target RNA encodes a truncated, elongated, mutated, or misfolded protein, the iADAR-ON can deaminate the target A in the target RNA, and result in a functional, full-length, correctly-folded and/or wild-type protein by reversal of a missense mutation, an early stop codon, aberrant splicing, or alternative splicing in the target RNA. In some embodiments, the iADAR-ON acts on a target RNA that is a regulatory RNA, and the iADAR-ON results in the deamination of the target A to effectuate a change in the expression of a downstream molecule regulated by the target RNA. For example, as disclosed herein, where the STOP codon is eliminated, the downstream GOI to the STOP codon is expressed (e.g., target activation construct or TAC), or alternatively, where the STOP is downstream of a GOI and upstream (e.g., 5′) of a polyA tail, the mRNA of the GOI is degraded.

In some embodiments, the iADAR can be used in any gene editing method where the at least one stop codon (e.g., ds-STOP codon) is inserted into a target nucleic acid sequence, for example, using gene editing methodologies such as CRISPR systems. While examples herein show exemplary RNA Target inactivation constructs (TIC) and RNA target activation constructs (TAC), it is contemplated herein that iADAR can be used in natural systems, circular RNA systems, ceDNA, etc. e.g., in which the at least one stop codon (e.g., ds-STOP codon) is inserted into the target nucleic acid sequence using gene editing methodologies. In some embodiments, there is provided an edited RNA or a host cell having an edited RNA produced by any one of the methods of RNA editing as described above.

In one aspect, described herein is a method of modulating RNA expression (e.g., RNA translation) in a cell, the method comprising contacting the cell with at least one fusion protein (e.g., iAD, iADAR) as described herein. In one aspect, described herein is a method of modulating RNA expression (e.g., RNA translation) in a cell, the method comprising contacting the cell with at least one synthetic RNA molecule (e.g., TIC, TAC) as described herein. In one aspect, described herein is a method of modulating RNA expression (e.g., RNA translation) in a cell, the method comprising contacting the cell with at least one nucleic acid as described herein. In one aspect, described herein is a method of modulating RNA expression (e.g., RNA translation) in a cell, the method comprising contacting the cell with at least one vector as described herein. In one aspect, described herein is a method of modulating RNA expression (e.g., RNA translation) in a cell, the method comprising contacting the cell with at least one system (e.g., iADAR and synthetic TIC or TAC RNA) as described herein. In one aspect, described herein is a method of modulating RNA expression (e.g., RNA translation) in a cell, the method comprising contacting the cell with at least one composition as described herein. In one aspect, described herein is a method of modulating RNA expression (e.g., RNA translation) in a cell, the method comprising contacting the cell with at least one pharmaceutical composition as described herein.

In some embodiments, the method further comprises contacting the cell with at least one inducer of the first and second binding pairs. In some embodiments, the method further comprises contacting the cell with at least one cleavage inducer.

In some embodiments, the methods described herein relate to treating a subject having or diagnosed as having a disease or disorder, such as cancer or an infectious disease. Subjects having cancer can be identified by a physician using current methods of diagnosing cancer. Symptoms and/or complications of cancer which characterize these conditions and aid in diagnosis are well known in the art. A family history of cancer, or exposure to risk factors for cancer can also aid in determining if a subject is likely to have cancer or in making a diagnosis of cancer.

Subjects having an infectious disease can be identified by a physician using current methods of an infectious disease. Symptoms and/or complications of an infectious disease which characterize these conditions and aid in diagnosis are well known in the art. A family history of infectious disease, or exposure to risk factors for infectious disease can also aid in determining if a subject is likely to have an infectious disease or in making a diagnosis of an infectious disease.

In one aspect, the present application provides a method for treating or preventing a disease or condition in an individual, comprising editing a target RNA associated with the disease or condition in a cell of the individual according to any one of the methods for RNA editing as described above. In some embodiments, the method comprises editing the target RNA in the cell ex vivo. In some embodiments, the method comprises administering a cell comprised the edited target RNA to the individual. In some embodiments, the method comprises administering to the individual an effective amount of the ADAR-recruiting RNA (dRNA) or construct encoding the dRNA. In some embodiments, the method further comprises introducing to the cell the ADAR or a construct (e.g., viral vector, a nucleic acid) encoding the ADAR. In some embodiments, the method further comprises administering to the individual the ADAR or a construct (e.g., viral vector, a nucleic acid) encoding the ADAR. In some embodiments, the disease or condition is a hereditary genetic disease. In some embodiments, the disease or condition is associated with one or more acquired genetic mutations, e.g., drug resistance. In some embodiments, the disease or condition is cancer. In some embodiments, the disease or condition is an infectious disease, such a viral, bacterial, or fungal infection.

The compositions described herein can be administered to a subject having or diagnosed as having a disease or disorder, such as cancer or an infectious disease. In some embodiments, the methods described herein comprise administering an effective amount of compositions described herein to a subject in order to alleviate a symptom of a disease or disorder, such as cancer or an infectious disease. As used herein, “alleviating a symptom” is ameliorating any condition or symptom associated with the a disease or disorder, such as cancer or an infectious disease. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

In one aspect, described herein is a method of treating a cancer or microbial infection in a subject in need thereof, the method comprising administering an effective amount of at least one fusion protein (e.g., iAD, iADAR) as described herein. In one aspect, described herein is a method of treating a cancer or microbial infection in a subject in need thereof, the method comprising administering an effective amount of at least one synthetic RNA molecule (e.g., TIC, TAC) as described herein. In one aspect, described herein is a method of treating a cancer or microbial infection in a subject in need thereof, the method comprising administering an effective amount of at least one nucleic acid as described herein. In one aspect, described herein is a method of treating a cancer or microbial infection in a subject in need thereof, the method comprising administering an effective amount of at least one vector as described herein. In one aspect, described herein is a method of treating a cancer or microbial infection in a subject in need thereof, the method comprising administering an effective amount of at least one system (e.g., iADAR and synthetic TIC or TAC RNA) as described herein. In one aspect, described herein is a method of treating a cancer or microbial infection in a subject in need thereof, the method comprising administering an effective amount of at least one composition as described herein. In one aspect, described herein is a method of treating a cancer or microbial infection in a subject in need thereof, the method comprising administering an effective amount of at least one pharmaceutical composition as described herein.

In some embodiments, the treatment method further comprises administering at least one inducer of the first and second binding pairs. In some embodiments, the treatment method further comprises administering at least one cleavage inducer. In some embodiments, the inducer or cleavage inducer is administered after the nucleic acid encoding the fusion protein and/or the nucleic acid encoding the RNA molecule.

In one aspect, described herein is a method for treating a cancer or microbial infection in a subject in need thereof, the method comprising administering an effective amount of (a) a nucleic acid encoding for an RNA molecule comprising: (i) a first open reading frame encoding for a fusion protein; (ii) a double-stranded region comprising: (A) at least one stop codon; and (B) a binding motif for RBD capable of being bound by an RNA-binding domain; and (iii) a second open reading frame encoding for an effector protein.

In one aspect, described herein is a method for treating a cancer or microbial infection in a subject in need thereof, the method comprising administering an effective amount of (a) a nucleic acid encoding a fusion protein comprising an RNA-binding domain linked to a deaminase domain of an adenosine deaminase; and (b) a nucleic acid encoding for an RNA molecule comprising: (i) an open reading frame encoding for an effector protein; (ii) a double-stranded region comprising: (A) at least one stop codon; and (B) a binding motif for RBD capable of being bound by an RNA-binding domain; and (iii) a poly-A tail.

In some embodiments, the effector protein comprises an antigen-binding domain for a cancer antigen. In some embodiments, the effector protein comprises an antigen-binding domain for microbial antigen.

In some embodiments, the fusion protein administered in a treatment method comprises an RNA-binding domain linked to a deaminase domain of an adenosine deaminase. In some embodiments, the fusion protein comprises: (a) an RNA-binding domain; (b) a first portion of a deaminase domain of an adenosine deaminase; (c) a first member of a binding pair; (d) a second portion of the deaminase domain; and/or (e) a second member of a binding pair. In some embodiments, the fusion protein administered in a treatment method comprises (a) an RNA-binding domain; (b) a first portion of a deaminase domain of an adenosine deaminase; (c) a first member of a binding pair; (d) a second portion of the deaminase domain; (e) a cleavable linker; and/or (f) a second member of a binding pair.

### VIII. Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal, e.g., for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a disease or disorder. A subject can be male or female.

A subject can be one who has been previously diagnosed with or identified as suffering from or having a condition in need of treatment or one or more complications related to such a condition, and optionally, have already undergone treatment for a condition to be treated, or the one or more complications related to a condition to be treated.

A “subject in need” of treatment for a particular condition can be a subject having that condition, diagnosed as having that condition, or at risk of developing that condition.

The term “effective amount” as used herein refers to the amount of (a) fusion protein, (b) RNA molecule, (c) nucleic acid, (d) vector, (e) system, (f) cell, (g) composition, and/or (g) pharmaceutical composition needed to alleviate at least one or more symptom of a disease or disorder in a subject in need thereof, and relates to a sufficient amount to provide the desired effect. The term “therapeutically effective amount” therefore refers to an amount of (a) fusion protein, (b) RNA molecule, (c) nucleic acid, (d) vector, (e) system, (f) cell, (g) composition, and/or (g) pharmaceutical composition that is sufficient to provide a particular effect, e.g., anti-cancer, e.g., anti-infectious disease, effect when administered to atypical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the minimal effective dose and/or maximal tolerated dose. The dosage can vary depending upon the dosage form employed and the route of administration utilized. A therapeutically effective dose can be estimated initially from cell culture assays. Also, a dose can be formulated in animal models to achieve a dosage range between the minimal effective dose and the maximal tolerated dose. The effects of any particular dosage can be monitored by a suitable bioassay, e.g., assay for tumor growth and/or size among others. The dosage can be determined by a physician and adjusted, as necessary, to suit observed effects of the treatment.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested by one of ordinary skill in the art to confirm that a desired activity, e.g. elimination of a STOP codon and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a polypeptide which retains at least 50% of the wild-type reference polypeptide's activity. A functional fragment can comprise conservative substitutions of the sequences disclosed herein.

In some embodiments, the polypeptide described herein can be a variant of a polypeptide sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a protein or fragment thereof that retains activity of the native or reference polypeptide. A wide variety of, for example, PCR-based, site-specific mutagenesis approaches are known in the art and can be applied by the ordinarily skilled artisan to generate and test artificial variants.

A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

A variant amino acid sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, similar to a native or reference sequence. As used herein, “similarity” refers to an identical amino acid or a conservatively substituted amino acid, as described herein. Accordingly, the percentage of “sequence similarity” is the percentage of amino acids which is either identical or conservatively changed; e.g., “sequence similarity”=(% sequence identity)+(% conservative changes). It should be understood that a sequence that has a specified percent similarity to a reference sequence necessarily encompasses a sequence with the same specified percent identity to that reference sequence. The skilled person will be aware of various computer programs, using different mathematical algorithms, that are available to determine the identity or similarity between two sequences. For instance, use can be made of a computer program employing the Needleman and Wunsch algorithm (Needleman et al. (1970)); the GAP program in the Accelrys GCG software package (Accelerys Inc., San Diego U.S.A.); the algorithm of E. Meyers and W. Miller (Meyers et al. (1989)) which has been incorporated into the ALIGN program (version 2.0); or more preferably the BLAST (Basic Local Alignment Tool using default parameters); see e.g., U.S. Pat. No. 10,023,890, the content of which is incorporated by reference herein in its entirety.

As used herein, the phrase “maintains the same function”, when used in reference to an enzyme, catalyzes the same reaction as a reference enzyme. When used in reference to an ADAR or AR, it changes an A to an I in the same molecule, substance, or factor.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. A wide variety of, site-specific mutagenesis approaches, e.g., Kunkel's method, cassette mutagenesis, PCR site-directed mutagenesis (e.g., traditional PCR, primer extension, or inverse PCR), whole plasmid mutagenesis, in vivo site-directed mutagenesis, CRISPR/Cas-guided mutagenesis, are known in the art and can be applied by the ordinarily skilled artisan to introduce mutations into specific nucleic acid loci. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); Braman, Jeff, ed. (2002) In Vitro Mutagenesis Protocols, Methods in Molecular Biology, Vol. 182 (2nd ed.); Khudyakov and Fields (2002), Artificial DNA: Methods and Applications, CRC Press; Hsu et al. (2014), Cell 157 (6): 1262-78; Cerchione et al. (2020) PLOS ONE 15 (4): e0231716; and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA, including closed ended DNA (ceDNA) or other circular DNA systems. Suitable RNA can include, e.g., mRNA and circular RNA constructs.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (e.g., mRNA) or antisense RNA derived from a nucleic acid fragment or fragments and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” refers to the nucleic acid sequence which is transcribed (DNA) to RNA in vitro or in vivo when operably linked to appropriate regulatory sequences. The gene may or may not include regions preceding and following a coding region, e.g. 5′ untranslated (5′UTR) or “leader” sequences and 3′ UTR or “trailer” sequences, as well as intervening sequences (introns) between individual coding segments (exons).

“In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, the iAD, e.g., iADAR described herein is exogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes a substance, such as a polypeptide or nucleic acid that is not naturally found or expressed in a given cell in its natural environment.

In some embodiments of any of the aspects, the iAD, e.g., iADAR2 comprises at least one functional heterologous gene. As used herein, the term “heterologous” refers to that which is not endogenous to, or naturally occurring in, a referenced sequence, molecule (including e.g., a protein), virus, cell, tissue, or organism. For example, a heterologous sequence of the present disclosure can be derived from a different species, or from the same species but substantially modified from an original form. Also for example, a nucleic acid sequence that is not normally expressed in a cell or a virus is a heterologous nucleic acid sequence with regard to that cell or virus. The term “heterologous” can refer to DNA, RNA, or protein that does not occur naturally as part of the organism in which it is present or which is found in a location or locations in the genome that differ from that in which it occurs in nature. It is DNA, RNA, or protein that is not endogenous to the virus or cell and has been artificially introduced into the virus or cell.

In some embodiments, a nucleic acid encoding a polypeptide as described herein (e.g. an iAD, e.g., iADAR2 polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art. Non-limiting examples of a viral vector of this invention include an AAV vector, an adenovirus vector, a lentivirus vector, a retrovirus vector, a herpesvirus vector, an alphavirus vector, a poxvirus vector, a baculovirus vector, and a chimeric virus vector.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in or within nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, transfection, transduction, perfusion, injection, or other delivery method known to one skilled in the art. In some embodiments, contacting comprises physical human activity, e.g., an injection; an act of dispensing, mixing, and/or decanting; and/or manipulation of a delivery device or machine.

In some embodiments of any of the aspects, the cells can be maintained in culture. As used herein, “maintaining” refers to continuing the viability of a cell or population of cells. A maintained population of cells will have at least a subpopulation of metabolically active cells.

As used herein, the term “specific binding” refers to a chemical or physical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure of the iAD, e.g., iADAR2 without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:

1. A fusion protein comprising:
(a) a first portion of a deaminase domain (DD) of an adenosine deaminase;
(b) a first member of a binding pair associated with the first portion of the DD;
(c) a second portion of the DD; and
(d) a second member of a binding pair associated with the second portion of the DD,
   wherein the first member of the binding pair is capable of binding to the second member of the binding pair in the absence of an inducer, resulting in allosteric inhibition of the first and second portions of the DD, and
   wherein the first member of the binding pair is not capable of binding to the second member of the binding pair in the presence of the inducer, resulting in activation of the first and second portions of the DD.

2. The fusion protein of paragraph 1, wherein in the allosteric inhibition of the first and second portions of the DD comprises deformation of the inositol hexaphosphate (IP6) binding pocket of first and second portions of the DD.

3. The fusion protein of paragraph 1, wherein in the activation of the first and second portions of the DD comprises deaminase activity.

4. The fusion protein of paragraph 1, further comprising an RNA-binding domain.

5. The fusion protein of paragraph 1, wherein the RNA-binding domain (RBD) is capable of binding to a binding motif for the RBD on an RNA molecule.

6. The fusion protein of paragraph 1, wherein the deaminase domain is capable of deamination of an adenosine nucleotide into an inosine nucleotide in an RNA molecule.

7. The fusion protein of paragraph 1, wherein the deaminase domain is capable of converting at least one stop codon into at least one non-stop codon.

8. The fusion protein of paragraph 1, wherein the deaminase domain is capable of converting UAG, UGA, or UAA stop codons to UIG, UGI, or UII non-stop codons, respectively.

9. The fusion protein of paragraph 1, wherein the deaminase domain is capable of converting at least one start codon into at least one non-start codon.

10. The fusion protein of paragraph 1, wherein the deaminase domain is capable of converting an AUG start codon to an JUG non-start codon.

11. The fusion protein of paragraph 1, wherein the deaminase domain is capable of converting at least one start codon into at least one non-start codon.

12. The fusion protein of paragraph 1, wherein the deaminase domain is capable of converting an AUA non-start codon to an AUI start codon.

13. The fusion protein of paragraph 1, wherein the deaminase domain is capable of converting at least one sense codon encoding a first amino acid into at least one mutated sense codon encoding a second amino acid.

14. The fusion protein of paragraph 1, wherein the deaminase domain is capable of converting at least one sense codon into at least one mutated sense codon, as selected from Table 15.

15. The fusion protein of paragraph 1, wherein the adenosine deaminase comprises Adenosine Deaminase Acting on RNA (ADAR), Adenosine Deaminase TRNA Specific (ADAT), or Adenosine Deaminase Domain Containing (ADAD).

16. The fusion protein of paragraph 1, wherein the adenosine deaminase comprises Adenosine Deaminase Acting on RNA (ADAR).

17. The fusion protein of paragraph 16, wherein the ADAR is ADAR1, ADAR2, or ADAR3.

18. The fusion protein of paragraph 16, wherein the ADAR is ADAR1 or ADAR2.

19. The fusion protein of paragraph 16, wherein the ADAR is ADAR2.

20. The fusion protein of paragraph 1, wherein in the presence of the inducer, the DD is constitutively active.

21. The fusion protein of paragraph 1, wherein in the presence of the inducer, the ADAR deaminase domain is constitutively active.

22. The fusion protein of paragraph 20 or 21, wherein the constitutively active deaminase domain comprises: an E1008Q mutation in ADAR1; an E488Q mutation in ADAR2; or an E527Q mutation in ADAR3.

23. The fusion protein of paragraph 1, wherein the DD comprises at least one mutation in the IP6 binding pocket that decreases background activity.

24. The fusion protein of paragraph 1, wherein the ADAR comprises at least one mutation in the IP6 binding pocket that decreases background activity.

25. The fusion protein of paragraph 23 or 24, wherein the at least one mutation is in an amino acid residue selected from the group consisting of: T375, R400, R522, Y658, K662, Y668, K672, V688, K690, F697, and L699.

26. The fusion protein of paragraph 23 or 24, wherein the at least one mutation is selected from the group consisting of: T375G, R400K, R522M, K662R, K662M, K672R, K672M, V688A, V688G, K690R, K690M, F697Y, F697L, F697I, F697V, F697A, F697G, L699V, L699A, and L699G.

27. The fusion protein of paragraph 1, wherein the adenosine deaminase comprises Adenosine Deaminase TRNA Specific (ADAT).

28. The fusion protein of paragraph 27, wherein the ADAT is ADAT1.

29. The fusion protein of paragraph 1, wherein the adenosine deaminase comprises Adenosine Deaminase Domain Containing (ADAD).

30. The fusion protein of paragraph 29, wherein the ADAD is ADAD1 or ADAD2.

31. The fusion protein of any one of paragraphs 1-30, wherein the ADAR, ADAT, or ADAD is a mammalian adenosine deaminase.

32. The fusion protein of any one of paragraphs 1-31, wherein the ADAR, ADAT, or ADAD is a human adenosine deaminase.

33. The fusion protein of paragraph 4, wherein the RNA-binding domain is selected from the group consisting of MCP, PCP, λN, and HIV tat.

34. The fusion protein of any one of paragraphs 1-33, comprising from N-terminus to C-terminus:
(a) the RNA-binding domain;
(b) the first portion of the deaminase domain;
(c) the first member of the binding pair;
(d) the second portion of the deaminase domain; and
(e) the second member of the binding pair.

35. The fusion protein of any one of paragraphs 1-34, wherein the first and second portions of the deaminase domain are split at an RNA binding loop.

36. The fusion protein of paragraph 35, wherein the RNA binding loop is the 5′ RNA binding loop (RBL) of ADAR1, ADAR2, ADAR3, ADAD1, or ADAD2.

37. The fusion protein of paragraph 35, wherein the RNA binding loop comprises:

(a) residues G969 to K999 of ADAR1:
(SEQ ID NO: 134)
GALFDKSCSDRAMESTESRHYPVFENPKQGK of ADAR1;
(b) residues A454 to Q479 of ADAR2:
(SEQ ID NO: 135)
ARIFSPHEPILEEPADRHPNRKARGQ;
(c) residues A493 to H518 of ADAR3:
(SEQ ID NO: 136)
ARLHSPYEITTDLHSSKHLVRKFRGH;
(d) residues A334 to K365 of ADAD1:
(SEQ ID NO: 137)
AQIKSQLRLNPHSISAFEANEELCLHVAVEGK;
or
(e) residues A347 to Q375 of ADAD2:
(SEQ ID NO: 138)
AARDIYLPPTSEGGLPHSPPMRLQAHVLGQ.

38. The fusion protein of paragraph 35, wherein the RNA binding loop comprises:

(SEQ ID NO: 139)
(a) residues K974 to S986 of ADAR1: KSCSDRAMES;
(SEQ ID NO: 140)
(b) residues F457 to D469 of ADAR2: FSPHEPILEEPAD;
(SEQ ID NO: 141)
(c) residues P498 to S508 of ADAR3: PYEITTDLHSS;
(SEQ ID NO: 142)
(d) residues Q339 to P344 of ADADI: QLRLNP;
or
(SEQ ID NO: 143)
(e) residues P352 to P360 of ADAD2: PPTSEGGLP

39. The fusion protein of paragraph 35, wherein the first and second portions of the deaminase domain are split between:
(a) residues S977 and D978 of ADAR1;
(b) residues T984 and E985 of ADAR1;
(c) residues A468 and D469 of ADAR2;
(d) residues S507 and S508 of ADAR3;
(e) residues L340 and R341 of ADAD1; or
(f) residues G357 and G358 of ADAD2.

40. The fusion protein of any one of paragraphs 1-39, comprising from N-terminus to C-terminus:
(a) the RNA-binding domain;
(b) the first member of the binding pair;
(c) the first portion of deaminase domain;
(d) the second portion of the deaminase domain; and
(e) the second member of the binding pair.

41. The fusion protein of any one of paragraphs 1-40, wherein the first and second members of the binding pair are Bad and Bcl-xL, and the inducer of the first and second binding pairs is A-1331852 or ABT-737.

42. The fusion protein of any one of paragraphs 1-40, wherein the first and second members of the binding pair are Bim and Bcl-xL, and the inducer of the first and second binding pairs is A-1331852.

43. The fusion protein of any one of paragraphs 1-40, wherein the first and second members of the binding pair are MS1 and MCL-1, and the inducer of the first and second binding pairs is S63845.

44. The fusion protein of any one of paragraphs 1-43, wherein the first and second members of the binding pair are a repressible protease and a protease-binding peptide, and the inducer of the first and second binding pairs is an inhibitor of the repressible protease.

45. The fusion protein of paragraph 44, wherein the repressible protease is selected from the group consisting of: HIV protease, HCV protease, and SARS-CoV2 protease.

46. The fusion protein of paragraph 44, wherein the protease-binding peptide comprises a domain that specifically binds to the HIV protease, HCV protease, or SARS-CoV2 protease.

47. The fusion protein of any one of paragraphs 44-46, wherein the repressible protease is NS3 protease from Hepatitis C virus (HCV).

48. The fusion protein of any one of paragraphs 44-47, wherein the protease-binding peptide is selected from the group consisting of: K5-66 peptide, K5-66-A peptide, K5-66-B peptide, K6-10 peptide, K6-10-A peptide, K6-10-B peptide, K5-66-R peptide, CP5-46 peptide, CP5-46-4D5E peptide, CP5-46-A peptide, CP5-46A-4D5E peptide, Ant-CP5-46A-4D5E peptide, and ANR peptide.

49. The fusion protein of any one of paragraphs 44-48, wherein the protease inhibitor is selected from the group consisting of: grazoprevir, danoprevir, simeprevir, asunaprevir, ciluprevir, boceprevir, sovaprevir, paritaprevir, ombitasvir, paritaprevir, ritonavir, dasabuvir, and telaprevir.

50. The fusion protein of any one of paragraphs 44-49, wherein the repressible protease is NS3 protease from Hepatitis C virus (HCV); the protease-binding peptide is CP5-46-4D5E, and/or the protease inhibitor is grazoprevir.

51. The fusion protein of any one of paragraphs 44-50, wherein the repressible protease is NS3 protease from Hepatitis C virus (HCV) type 1b.

52. The fusion protein of any one of paragraphs 1-51, wherein:
(a) the first member of the binding pair comprises an antigen-binding domain,
(b) the second member of the binding pair comprises a first antigen, and
(c) the inducer of the first and second binding pairs comprises a second antigen;
   wherein the antigen-binding domain is capable of binding to the second antigen with a similar or higher affinity than to the first antigen.

53. The fusion protein of any one of paragraphs 1-52, wherein:
(a) the first member of the binding pair comprises an anti-ALFA antigen binding domain,
(b) the second member of the binding pair comprises a first ALFA antigen, and
(c) the inducer of the first and second binding pairs comprises a second ALFA antigen;
   wherein the anti-ALFA antigen binding domain is capable of binding to the second ALFA antigen or variants thereof.

54. The fusion protein of paragraph 53, wherein the anti-ALFA antigen binding domain is capable of binding to the second ALFA antigen or variants thereof with a similar or higher affinity than to the first ALFA antigen.

55. The fusion protein of any one of paragraphs 1-54, wherein the fusion protein further comprises a cleavable linker between the second portion of the deaminase domain the second member of the binding pair.

56. The fusion protein of paragraph 55, wherein the cleavable linker comprises at least one protease cleavage site.

57. A fusion protein comprising:
(a) a first portion of a deaminase domain (DD) of an adenosine deaminase;
(b) a first member of a binding pair associated with the first portion of the DD;
(c) a second portion of the DD;
(d) a cleavable linker; and
(e) a second member of a binding pair associated with the cleavable linker;
   wherein the first member of the binding pair is capable of binding to the second member of the binding pair in the absence of a cleavage inducer, resulting in allosteric inhibition of the first and second portions of the DD, and
   wherein the cleavable linker is cleaved in the presence of the cleavage inducer, resulting in activation of the first and second portions of the DD.

58. The fusion protein of paragraph 57, wherein in the allosteric inhibition comprises deformation of the inositol hexaphosphate (IP6) binding pocket of first and second portions of the DD.

59. The fusion protein of paragraph 57, wherein in the activation of the first and second portions of the DD comprises deaminase activity.

60. The fusion protein of paragraph 57, wherein the cleavage inducer for the cleavable linker is light, sound, chemical, or an enzyme.

61. The fusion protein of paragraph 57, further comprising an RNA-binding domain (RBD).

62. The fusion protein of paragraph 61, wherein the RNA-binding domain (RBD) is capable of binding to a binding motif for the RBD on an RNA molecule.

63. The fusion protein of any one of paragraphs 57-62, wherein:
(a) the first member of the binding pair comprises a SpyCatcher domain,
(b) the second member of the binding pair comprises a Spy tag,
(c) the cleavable linker comprises Tobacco Etch Virus (TEV) cut site cleavable by a TEV protease, and
(d) the cleavage inducer comprises light.

64. A fusion protein comprising an RNA-binding domain linked to a deaminase domain of an adenosine deaminase.

65. The fusion protein of paragraph 64, wherein the RNA-binding domain is N-terminal of the deaminase domain.

66. The fusion protein of paragraph 65, wherein the RNA-binding domain is C-terminal of the deaminase domain.

67. A fusion protein comprising:
(a) a first portion of a deaminase domain (DD) of an adenosine deaminase;
(b) a first member of a first binding pair associated with the first portion of the DD;
(c) a second portion of the DD;
(d) a second member of a first binding pair associated with the second portion of the DD;
(e) a first member of a second binding pair associated with the first member of the first binding pair; and
(f) a second member of the second binding pair associated with the second member of the first binding pair;
   wherein the first member of the first binding pair is capable of binding to the second member of the first binding pair in the absence of a first inducer, resulting in allosteric inhibition of the first and second portions of the DD;
   wherein the first member of the first binding pair is not capable of binding to the second member of the first binding pair in the presence of the first inducer, resulting in activation of the first and second portions of the DD;
   wherein the first member of the second binding pair is capable of binding to the second member of the second binding pair in the absence of a second inducer, resulting in allosteric inhibition of the first and second portions of the DD; and
   wherein the first member of the second binding pair is not capable of binding to the second member of the second binding pair in the presence of the second inducer, resulting in activation of the first and second portions of the DD.

68. A fusion protein comprising:
(a) a first portion of a deaminase domain (DD) of an adenosine deaminase;
(b) a repressible protease associated with the first portion of the DD;
(c) a second portion of the DD; and
(d) a protease-binding peptide associated with the second portion of the DD,
   wherein the repressible protease is capable of binding to the protease-binding peptide in the absence of an inhibitor for the repressible protease, resulting in allosteric inhibition of the first and second portions of the DD, and
   wherein the repressible protease is not capable of binding to the protease-binding peptide in the presence of the inhibitor for the repressible protease, resulting in activation of the first and second portions of the DD.

69. A fusion protein comprising:
(a) a first portion of a deaminase domain (DD) of an adenosine deaminase;
(b) a repressible protease associated with the first portion of the DD;
(c) a second portion of the DD; and
(d) a protease cleavage site associated with the first and second portions of the DD;
   wherein the repressible protease is capable of binding to the protease cleavage site in the absence of an inhibitor for the repressible protease, resulting in cleavage of the protease cleavage site and inactivation of the first and second portions of the DD; and
   wherein the repressible protease is not capable of binding to the protease cleavage site in the presence of the inhibitor for the repressible protease, resulting in activation of the first and second portions of the DD.

70. The fusion protein of paragraph 68 or 69, wherein the repressible protease is selected from the group consisting of: HIV protease, HCV protease, and SARS-CoV2 protease.

71. The fusion protein of any one of paragraphs 68-70, wherein the protease-binding peptide comprises a domain that is capable of specifically binding to the HIV protease, HCV protease, or SARS-CoV2 protease.

72. The fusion protein of any one of paragraphs 68-71, wherein the repressible protease is NS3 protease from Hepatitis C virus (HCV).

73. The fusion protein of any one of paragraphs 68-72, wherein the protease-binding peptide is selected from the group consisting of: K5-66 peptide, K5-66-A peptide, K5-66-B peptide, K6-10 peptide, K6-10-A peptide, K6-10-B peptide, K5-66-R peptide, CP5-46 peptide, CP5-46-4D5E peptide, CP5-46-A peptide, CP5-46A-4D5E peptide, Ant-CP5-46A-4D5E peptide, and ANR peptide.

74. The fusion protein of any one of paragraphs 68-73, wherein the protease inhibitor is selected from the group consisting of: grazoprevir, danoprevir, simeprevir, asunaprevir, ciluprevir, boceprevir, sovaprevir, paritaprevir, ombitasvir, paritaprevir, ritonavir, dasabuvir, and telaprevir.

75. The fusion protein of any one of paragraphs 68-74, wherein the repressible protease is NS3 protease from Hepatitis C virus (HCV); the protease-binding peptide is CP5-46-4D5E, and/or the protease inhibitor is grazoprevir.

76. The fusion protein of any one of paragraphs 68-75, wherein the repressible protease is NS3 protease from Hepatitis C virus (HCV) type 1b.

77. The fusion protein of any one of paragraphs 68-76, wherein the protease cleavage site is a NS3/NS4A cleavage site, a NS4A/NS4B cleavage site, a NS4B/NS5A cleavage site, or a NS5A/NS5B cleavage site.

78. A fusion protein comprising from N terminus to C terminus:
a) ADAR2-DDN;
b) NS3 binding peptide;
c) ADAR2-DDC; and
d) HCV NS3 protease from genotype 1B.

79. The fusion protein of paragraph 78, wherein the ADAR2-DDN comprises residues Q316 to A568 of ADAR2.

80. The fusion protein of paragraph 78, wherein the NS3 binding peptide comprises CP5-46-4D5E.

81. The fusion protein of paragraph 78, wherein the ADAR2-DDC comprises residues D569 to T700 of ADAR2.

82. The fusion protein of paragraph 78, wherein the fusion protein further comprises a tandem-dimer MS2 coat protein (tdMCP) at the N-terminus.

83. An RNA molecule comprising:
(a) a double-stranded region comprising:
   (i) at least one target codon; and
   (ii) an RNA binding motif capable of being bound by an RNA-binding domain; and
(b) at least one open reading frame, wherein the at least one open reading frame is operatively linked to the double-stranded region.

84. The RNA molecule of paragraph 83, wherein the at least one open reading frame encodes for the fusion protein of any one of paragraphs 1-82.

85. The RNA molecule of paragraph 83, wherein the at least one open reading frame encodes for at least one effector protein.

86. An RNA molecule comprising:
(a) a first open reading frame;
(b) a double-stranded region comprising:
   (i) at least one target codon; and
   (ii) an RNA binding motif capable of being bound by an RNA-binding domain; and
(c) a second open reading frame, wherein the second open reading frame is operatively linked to the double-stranded region.

87. The RNA molecule of paragraph 86, wherein the first open reading frame encodes for a first polypeptide, and the second reading frame encodes for a second polypeptide.

88. The RNA molecule of paragraph 86, wherein the first open reading frame encodes for a first portion of a polypeptide, and the second reading frame encodes for a second portion of the polypeptide.

89. The RNA molecule of paragraph 86, wherein the first open reading frame encodes for the fusion protein of any one of paragraphs 1-82.

90. The RNA molecule of paragraph 86, wherein the second open reading frame encodes for at least one effector protein.

91. The RNA molecule of paragraph 86, wherein the second open reading frame encodes for the fusion protein of any one of paragraphs 1-82.

92. The RNA molecule of paragraph 86, wherein the first open reading frame encodes for at least one effector protein.

93. The RNA molecule of paragraph 86, wherein an internal ribosome entry site (IRES) is located between the first and second open reading frames.

94. An RNA molecule comprising:
(a) an open reading frame;
(b) a double-stranded region comprising:
   (i) at least one target codon; and
   (ii) an RNA binding motif capable of being bound by an RNA-binding domain; and
(c) a poly-A tail.

95. The RNA molecule of paragraph 94, wherein the open reading frame encodes for the fusion protein of any one of paragraphs 1-82.

96. The RNA molecule of paragraph 94, wherein the open reading frame encodes for an effector protein.

97. The RNA molecule of any one of paragraphs 83-96, wherein the effector protein comprises an antigen-binding domain for a cancer antigen or a microbial antigen.

98. The RNA of any one of paragraphs 83-97, wherein the target codon comprises at least one adenosine nucleotide.

99. The RNA of any one of paragraphs 83-98, wherein the target codon is a stop codon, a start codon, a non-start codon, or a sense codon.

100. The RNA of paragraph 99, wherein the stop codon is capable of being converted into a non-stop codon.

101. The RNA molecule of paragraph 99 or 100, wherein the at least one stop codon comprises UAG.

102. The RNA molecule of paragraph 100, wherein the at least one non-stop codon comprises at least one tryptophan codon.

103. The RNA molecule of paragraph 102, wherein the at least one tryptophan codon comprises UGG.

104. The RNA of any one of paragraphs 99-103, wherein the stop codon is UAG, UGA, or UAA, and the non-stop codon is UIG, UGI, or UII, respectively.

105. The RNA of paragraph 99, wherein the start codon is capable of being converted into a non-start codon.

106. The RNA of paragraph 105, wherein the start codon is AUG, and the non-start codon is JUG.

107. The RNA of paragraph 99, wherein the non-start codon is capable of being converted into a non-start codon.

108. The RNA of paragraph 107, wherein the non-start codon is AUA, and the start codon is AUI.

109. The RNA of paragraph 99, wherein the sense codon encoding a first amino acid is capable of being converted into a mutated sense codon encoding a second amino acid.

110. The RNA of paragraph 109, wherein the sense codon and mutated sense codon are selected from Table 15.

111. The RNA molecule of paragraph 83-110, the RNA-binding domain comprises the RNA-binding domain of the fusion protein of any one of paragraphs 1-82.

112. The RNA molecule of paragraph 83-111, wherein the RNA binding motif is selected from the group consisting of MS2, PP7, BoxB, and TAR.

113. The RNA molecule of paragraph 83-112, wherein the RNA-binding domain is selected from the group consisting of MCP, PCP, λN, and HIV tat.

114. The RNA molecule of paragraph 83-113, wherein:
(a) the RNA-binding domain comprises MCP, and the RNA binding motif comprises MS2;
(b) the RNA-binding domain comprises PCP, and the RNA binding motif comprises PP7;
(c) the RNA-binding domain comprises λN, and the RNA binding motif comprises BoxB; or
(d) the RNA-binding domain comprises HIV Tat, and the RNA binding motif comprises TAR.

115. The RNA molecule of paragraph 83-114, wherein the double-stranded region of the RNA molecule comprises at least one hairpin.

116. The RNA molecule of paragraph 83-115, wherein the double-stranded region of the RNA molecule comprises one hairpin comprising the at least one target codon and the RNA binding motif.

117. The RNA molecule of paragraph 83-116, wherein the double-stranded region of the RNA molecule comprises a first hairpin comprising the at least one target codon and a second hairpin comprising the RNA binding motif.

118. A nucleic acid encoding the fusion protein of any one of paragraphs 1-82.

119. A nucleic acid encoding the RNA molecule of any one of paragraphs 83-117.

120. A nucleic acid encoding the fusion protein of any one of paragraphs 1-82 and the RNA molecule of any one of paragraphs 83-117.

121. The nucleic acid of paragraph 120, wherein the nucleic acid encoding the fusion protein and the nucleic acid encoding the RNA molecule are operably linked to a single promoter.

122. The nucleic acid of paragraph 120, wherein the nucleic acid encoding the fusion protein and the nucleic acid encoding the RNA molecule are each operably linked to a separate promoter.

123. The nucleic acid of any one of paragraphs 120-122, wherein the nucleic acid encoding the fusion protein is 5′ of the nucleic acid encoding the RNA molecule.

124. The nucleic acid of any one of paragraphs 120-122, wherein the nucleic acid encoding the fusion protein is 3′ of the nucleic acid encoding the RNA molecule.

125. The nucleic acid of any one of paragraphs 118-124, wherein the nucleic acid comprises DNA.

126. The nucleic acid of any one of paragraphs 118-125, wherein the nucleic acid comprises RNA.

127. A vector comprising the nucleic acid of any one of paragraphs 118-126.

128. A system for modulating RNA translation comprising the fusion protein of any one of paragraphs 1-82 and the RNA molecule of any one of paragraphs 83-117.

129. A system for modulating RNA translation comprising a first fusion protein of any one of paragraphs 1-82 and a second fusion protein of any one of paragraphs 1-82.

130. A system for modulating RNA translation comprising at least one fusion protein of any one of paragraphs 1-82 and at least one RNA molecule of any one of paragraphs 83-117.

131. A system for modulating RNA translation comprising
(a) a fusion protein comprising an RNA-binding domain linked to a deaminase domain of an adenosine deaminase; and
(b) an RNA molecule comprising:
   (i) a double-stranded region comprising:
      (A) at least one target codon; and
      (B) an RNA binding motif capable of being bound by an RNA-binding domain; and
      (ii) at least one open reading frame, wherein the at least one open reading frame is operatively linked to the double-stranded region.

132. The system of any one of paragraphs 131, wherein the target codon is a stop codon, a start codon, a non-start codon, or a sense codon.

133. The system of paragraph 131 or 132, wherein the deaminase domain is capable of converting the at least one stop codon into at least one non-stop codon.

134. The system of any one of paragraphs 131-133, wherein the reading frame is not translated when the at least one stop codon is present in the double-stranded region of the RNA molecule.

135. The system of any one of paragraphs 131-134, wherein the reading frame is translated when the at least one stop codon is converted into the at least one non-stop codon.

136. The system of any one of paragraphs 131-135, wherein the deaminase domain is capable of converting the at least one start codon into at least one non-start codon.

137. The system of any one of paragraphs 131-136, wherein the reading frame is translated when the at least one start codon is present in the double-stranded region of the RNA molecule.

138. The system of any one of paragraphs 131-137, wherein the reading frame is not translated when the at least one start codon is converted into the at least one non-start codon.

139. The system of any one of paragraphs 131-138, wherein the deaminase domain is capable of converting the at least one non-start codon into at least one start codon.

140. The system of any one of paragraphs 131-139, wherein the reading frame is not translated when the at least one non-start codon is present in the double-stranded region of the RNA molecule.

141. The system of any one of paragraphs 131-140, wherein the reading frame is translated when the at least one non-start codon is converted into the at least one start codon.

142. The system of any one of paragraphs 131-141, wherein the deaminase domain is capable of converting the at least one sense stop codon encoding a first amino acid into at least one mutated sense codon encoding a second amino acid.

143. A system for modulating RNA translation comprising:
(a) a fusion protein comprising an RNA-binding domain linked to a deaminase domain of an adenosine deaminase; and
(b) an RNA molecule comprising:
   (i) a first open reading frame;
   (ii) a double-stranded region comprising:
      (A) at least one target codon; and
      (B) an RNA binding motif capable of being bound by the RNA-binding domain of the fusion protein; and
   (iii) a second open reading frame.

144. The RNA of paragraph 143, wherein the target codon is a stop codon, a start codon, a non-start codon, or a sense codon.

145. The system of paragraph 143 or 144, wherein the deaminase domain is capable of converting the at least one stop codon into at least one non-stop codon.

146. The system of any one of paragraphs 143-145, wherein the second reading frame is not translated when the at least one stop codon is present in the double-stranded region of the RNA molecule.

147. The system of any one of paragraphs 143-146, wherein the second reading frame is translated when the at least one stop codon is converted into the at least one non-stop codon.

148. The system of any one of paragraphs 143-147, wherein the first open reading frame is translated when the at least one stop codon or the at least one non-stop codon is present in the double-stranded region of the RNA molecule.

149. The system of any one of paragraphs 143-148, wherein the deaminase domain is capable of converting the at least one start codon into at least one non-start codon.

150. The system of any one of paragraphs 143-149, wherein the second reading frame is translated when the at least one start codon is present in the double-stranded region of the RNA molecule.

151. The system of any one of paragraphs 143-150, wherein the second reading frame is not translated when the at least one start codon is converted into the at least one non-start codon.

152. The system of any one of paragraphs 143-151, wherein the first open reading frame is translated when the at least one start codon or the at least one non-start codon is present in the double-stranded region of the RNA molecule.

153. The system of any one of paragraphs 143-152, wherein the deaminase domain is capable of converting the at least one non-start codon into at least one start codon.

154. The system of any one of paragraphs 143-153, wherein the second reading frame is not translated when the at least one non-start codon is present in the double-stranded region of the RNA molecule.

155. The system of any one of paragraphs 143-154, wherein the second reading frame is translated when the at least one non-start codon is converted into the at least one start codon.

156. The system of any one of paragraphs 143-155, wherein the deaminase domain is capable of converting the at least one sense stop codon encoding a first amino acid into at least one mutated sense codon encoding a second amino acid.

157. The system of any one of paragraphs 143-156, wherein the first open reading frame encodes for a first polypeptide, and the second reading frame encodes for a second polypeptide.

158. The system of any one of paragraphs 143-157, wherein the first open reading frame encodes for a first portion of a polypeptide, and the second reading frame encodes for a second portion of the polypeptide.

159. The system of any one of paragraphs 143-158, wherein the first open reading frame encodes for the fusion protein of any one of paragraphs 1-82.

160. The system of any one of paragraphs 143-159, wherein the second open reading frame encodes for an effector protein.

161. The system of any one of paragraphs 143-160, wherein the effector protein comprises an antigen-binding domain for a cancer antigen or a microbial antigen.

162. A system for modulating RNA translation comprising:
(a) a fusion protein comprising an RNA-binding domain linked to a deaminase domain of an adenosine deaminase; and
(b) an RNA molecule comprising:
   (i) an open reading frame;
   (ii) a double-stranded region comprising:
      (A) at least one target codon; and
      (B) an RNA binding motif capable of being bound by the RNA-binding domain of the fusion protein; and
   (iii) a poly-A tail.

163. The RNA of paragraph 162, wherein the target codon is a stop codon, a start codon, a non-start codon, or a sense codon.

164. The system of paragraph 162 or 163, wherein the deaminase domain is capable of converting the at least one stop codon into at least one non-stop codon.

165. The system of any one of paragraphs 162-164, wherein the first open reading frame is translated when the at least one stop codon is present in the double-stranded region of the RNA molecule.

166. The system of any one of paragraphs 162-165, wherein the RNA molecule is degraded when the at least one stop codon is converted into the at least one non-stop codon.

167. A system for modulating RNA translation comprising:
(a) a fusion protein comprising:
   (i) an RNA-binding domain;
   (ii) a first portion of a deaminase domain (DD) of an adenosine deaminase;
   (iii) a first member of a binding pair associated with the first portion of the DD;
   (iv) a second portion of the DD; and
   (v) a second member of a binding pair associated with the second portion of the DD; and
(b) the RNA molecule of any one of paragraphs 83-117.

168. A system for modulating RNA translation comprising:
(a) a fusion protein comprising:
   (i) an RNA-binding domain;
   (ii) a first portion of a deaminase domain (DD) of an adenosine deaminase;
   (iii) a first member of a binding pair associated with the first portion of the DD;
   (iv) a second portion of the DD; and
   (v) a second member of a binding pair associated with the second portion of the DD; and
(b) an RNA molecule comprising:
   (i) a first open reading frame;
   (ii) a double-stranded region comprising:
      (A) at least one target codon; and
      (B) an RNA binding motif capable of being bound by the RNA-binding domain of the fusion protein; and
   (iii) a second open reading frame.

169. A system for modulating RNA translation comprising:
(a) a fusion protein comprising:
   (i) an RNA-binding domain;
   (ii) a first portion of a deaminase domain (DD) of an adenosine deaminase;
   (iii) a first member of a binding pair associated with the first portion of the DD;
   (iv) a second portion of the DD; and
   (v) a second member of a binding pair associated with the second portion of the DD; and
(b) an RNA molecule comprising:
   (i) an open reading frame;
   (ii) a double-stranded region comprising:
      (A) at least one target codon; and
      (B) an RNA binding motif capable of being bound by the RNA-binding domain of the fusion protein; and
   (iii) a poly-A tail.

170. The system of any one of paragraphs 167-169, wherein the target codon is a stop codon, a start codon, a non-start codon, or a sense codon.

171. The system of any one of paragraphs 128-170, the system further comprising an inducer of the first and second binding pairs.

172. The system of any one of paragraphs 128-171, wherein the first and second members of the binding pair of the fusion protein are capable of binding to each other in the absence of an inducer of the first and second binding pairs and reduce or prevent:
(a) the formation of the inositol hexaphosphate (IP6) binding pocket of first and second portions of the deaminase domain;
(b) deaminase activity of the first and second portions of the deaminase domain;
(c) conversion by the deaminase domain of the at least one target codon of the RNA molecule into at least one inosine-comprising codon;
(d) conversion by the deaminase domain of the at least one stop codon of the RNA molecule into at least one non-stop codon;
(e) conversion by the deaminase domain of the at least one start codon of the RNA molecule into at least one non-start codon;
(f) conversion by the deaminase domain of the at least one non-start codon of the RNA molecule into at least one start codon; and/or
(g) conversion by the deaminase domain of the at least one sense codon of the RNA molecule encoding for a first amino acid into at least one mutated codon encoding for a second amino acid.

173. The system of any one of paragraphs 128-172, wherein the first and second members of the binding pair of the fusion protein are not capable of binding to each other in the presence of an inducer of the first and second binding pairs, allowing for:
(a) the formation of the inositol hexaphosphate (IP6) binding pocket of first and second portions of the deaminase domain;
(b) deaminase activity of the first and second portions of the deaminase domain;
(c) conversion by the deaminase domain of the at least one target codon of the RNA molecule into at least one inosine-comprising codon;
(d) conversion by the deaminase domain of the at least one stop codon of the RNA molecule into at least one non-stop codon;
(e) conversion by the deaminase domain of the at least one start codon of the RNA molecule into at least one non-start codon;
(f) conversion by the deaminase domain of the at least one non-start codon of the RNA molecule into at least one start codon; and/or
(g) conversion by the deaminase domain of the at least one sense codon of the RNA molecule encoding for a first amino acid into at least one mutated sense codon encoding for a second amino acid.

174. The system of any one of paragraphs 128-173, wherein conversion by the deaminase domain of at least one target codon in the RNA molecule into at least one inosine-comprising codon increases or decreases:
(a) translation of the reading frame of the RNA molecule;
(b) translation of the second reading frame of the RNA molecule;
(c) degradation of the RNA molecule; and/or
(d) translation of a variant polypeptide encoded by the RNA molecule.

175. A system for modulating RNA translation comprising:
(a) a fusion protein comprising:
   (i) an RNA-binding domain;
   (ii) a first portion of a deaminase domain (DD) of an adenosine deaminase;
   (iii) a first member of a binding pair associated with the first portion of the DD;
   (iv) a second portion of the DD;
   (v) a cleavable linker; and
   (vi) a second member of a binding pair associated with the cleavable linker; and
(b) the RNA molecule of any one of paragraphs 83-117.

176. A system for modulating RNA translation comprising:
(a) a fusion protein comprising:
   (i) an RNA-binding domain;
   (ii) a first portion of a deaminase domain (DD) of an adenosine deaminase;
   (iii) a first member of a binding pair associated with the first portion of the DD;
   (iv) a second portion of the DD;
   (v) a cleavable linker; and
   (vi) a second member of a binding pair associated with the cleavable linker; and
(b) an RNA molecule comprising:
   (i) a first open reading frame;
   (ii) a double-stranded region comprising:
      (A) at least one target codon; and
      (B) an RNA binding motif capable of being bound by the RNA-binding domain of the fusion protein; and
   (iii) a second open reading frame.

177. A system for modulating RNA translation comprising:
(a) a fusion protein comprising:
   (i) an RNA-binding domain;
   (ii) a first portion of a deaminase domain (DD) of an adenosine deaminase;
   (iii) a first member of a binding pair associated with the first portion of the DD;
   (iv) a second portion of the DD;
   (v) a cleavable linker; and
   (vi) a second member of a binding pair associated with the cleavable linker; and
(b) an RNA molecule comprising:
   (i) an open reading frame;
   (ii) a double-stranded region comprising:
      (A) at least one target codon; and
      (B) an RNA binding motif capable of being bound by the RNA-binding domain of the fusion protein; and
   (iii) a poly-A tail.

178. The system of any one of paragraphs 175-177, the system further comprising a cleavage inducer.

179. The system of any one of paragraphs 175-178, wherein the cleavable linker is not cleaved in the absence of a cleavage inducer and reduces or prevents:
(a) the formation of the inositol hexaphosphate (IP6) binding pocket of first and second portions of the deaminase domain;
(b) deaminase activity of the first and second portions of the deaminase domain;
(c) conversion by the deaminase domain of the at least one target codon of the RNA molecule into at least one inosine-comprising codon;
(d) conversion by the deaminase domain of the at least one stop codon of the RNA molecule into at least one non-stop codon;
(e) conversion by the deaminase domain of the at least one start codon of the RNA molecule into at least one non-start codon;
(f) conversion by the deaminase domain of the at least one non-start codon of the RNA molecule into at least one start codon; and/or
(g) conversion by the deaminase domain of the at least one sense codon of the RNA molecule encoding for a first amino acid into at least one mutated sense codon encoding for a second amino acid.

180. The system of any one of paragraphs 175-179, wherein the cleavable linker is cleaved in the presence of a cleavage inducer and allows for:
(a) the formation of the inositol hexaphosphate (IP6) binding pocket of first and second portions of the deaminase domain;
(b) deaminase activity of the first and second portions of the deaminase domain;
(c) conversion by the deaminase domain of the at least one target codon of the RNA molecule into at least one inosine-comprising codon;
(d) conversion by the deaminase domain of the at least one stop codon of the RNA molecule into at least one non-stop codon;
(e) conversion by the deaminase domain of the at least one start codon of the RNA molecule into at least one non-start codon;
(f) conversion by the deaminase domain of the at least one non-start codon of the RNA molecule into at least one start codon; and/or
(g) conversion by the deaminase domain of the at least one sense codon of the RNA molecule encoding for a first amino acid into at least one mutated sense codon encoding for a second amino acid.

181. The system of any one of paragraphs 175-180, wherein conversion by the deaminase domain of at least one target codon in the RNA molecule into at least one inosine-comprising codon increases or decreases:
(a) translation of the reading frame of the RNA molecule;
(b) translation of the second reading frame of the RNA molecule;
(c) degradation of the RNA molecule; and/or
(d) translation of a variant polypeptide encoded by the RNA molecule.

182. The system of any one of paragraphs 175-181, wherein the target codon is a stop codon, a start codon, a non-start codon, or a sense codon.

183. The system of any one of paragraphs 128-182, wherein the at least one stop codon comprises UAG.

184. The system of any one of paragraphs 128-183, wherein the at least one non-stop codon comprises at least one tryptophan codon.

185. The system of any one of paragraphs 128-184, wherein the at least one tryptophan codon comprises UGG.

186. The system of any one of paragraphs 128-185, wherein the stop codon is UAG, UGA, or UAA, and the non-stop codon is UIG, UGI, or UII, respectively.

187. The system of any one of paragraphs 128-186, wherein the start codon is capable of being converted into a non-start codon.

188. The system of any one of paragraphs 128-187, wherein the start codon is AUG, and the non-start codon is JUG.

189. The system of any one of paragraphs 128-188, wherein the non-start codon is capable of being converted into a start codon.

190. The system of any one of paragraphs 128-189, wherein the non-start codon is AUA, and the start codon is AUI.

191. The system of any one of paragraphs 128-190, wherein the sense codon encoding a first amino acid is capable of being converted into a mutated sense codon encoding a second amino acid.

192. The system of any one of paragraphs 128-191, wherein the sense codon and mutated sense codon are selected from Table 15.

193. The system of any one of paragraphs 128-192, wherein:
(a) the RNA-binding domain comprises MCP, and the RNA binding motif comprises MS2;
(b) the RNA-binding domain comprises PCP, and the RNA binding motif comprises PP7;
(c) the RNA-binding domain comprises λN, and the RNA binding motif comprises BoxB; or
(d) the RNA-binding domain comprises HIV Tat, and the RNA binding motif comprises TAR.

194. The system of any one of paragraphs 128-193, wherein the double-stranded region of the RNA molecule comprises at least one hairpin.

195. The system of any one of paragraphs 128-194, wherein the double-stranded region of the RNA molecule comprises one hairpin comprising the at least one stop codon and the RNA binding motif.

196. The system of any one of paragraphs 128-195, wherein the double-stranded region of the RNA molecule comprises a first hairpin comprising the at least one stop codon and a second hairpin comprising the RNA binding motif.

197. A cell comprising:
(a) the fusion protein of any one of paragraphs 1-82;
(b) the RNA molecule of any one of paragraphs 83-117;
(c) the nucleic acid of any one of paragraphs 118-126;
(d) the vector of paragraph 127; and/or
(e) the system of any one of paragraphs 128-196.

198. A composition comprising at least one of:
(a) the fusion protein of any one of paragraphs 1-82;
(b) the RNA molecule of any one of paragraphs 83-117;
(c) the nucleic acid of any one of paragraphs 118-126;
(d) the vector of paragraph 127;
(e) the system of any one of paragraphs 128-196; and/or
(f) the cell of paragraph 197.

199. The composition of paragraph 198, the composition further comprising:
(a) an inducer of the first and second binding pairs;
(b) a cleavage inducer; and/or
(c) a protease inhibitor.

200. A pharmaceutical composition comprising a pharmaceutically compatible carrier and at least one of:
(a) the fusion protein of any one of paragraphs 1-82;
(b) the RNA molecule of any one of paragraphs 83-117;
(c) the nucleic acid of any one of paragraphs 118-126;
(d) the vector of paragraph 127;
(e) the system of any one of paragraphs 128-196; and/or
(f) the cell of paragraph 197.

201. The pharmaceutical composition of paragraph 200, the pharmaceutical composition further comprising:
(a) an inducer of the first and second binding pairs;
(b) a cleavage inducer; and/or
(c) a protease inhibitor.

202. A method of modulating RNA translation in a cell, the method comprising contacting the cell with:
(a) the fusion protein of any one of paragraphs 1-82;
(b) the RNA molecule of any one of paragraphs 83-117;
(c) the nucleic acid of any one of paragraphs 118-126;
(d) the vector of paragraph 127;
(e) the system of any one of paragraphs 128-196;
(f) the composition of any one of paragraphs 198-199; and/or
(g) the pharmaceutical composition of any one of paragraphs 200-201.

203. The method of paragraph 202, the method further comprising contacting the cell with:
(a) an inducer of the first and second binding pairs;
(b) a cleavage inducer; and/or
(c) a protease inhibitor.

204. A method for treating a cancer or microbial infection in a subject in need thereof, the method comprising administering an effective amount of:
(a) the fusion protein of any one of paragraphs 1-82;
(b) the RNA molecule of any one of paragraphs 83-117;
(c) the nucleic acid of any one of paragraphs 118-126;
(d) the vector of paragraph 127;
(e) the system of any one of paragraphs 128-196;
(f) the cell of paragraph 197;
(g) the composition of any one of paragraphs 198-199; and/or
(h) the pharmaceutical composition of any one of paragraphs 200-201.

205. The method of paragraph 204, the method further comprising contacting the cell with:
(a) an inducer of the first and second binding pairs;
(b) a cleavage inducer; and/or
(c) a protease inhibitor.

206. A method for treating a cancer or microbial infection in a subject in need thereof, the method comprising administering an effective amount of:
(a) a nucleic acid encoding for an RNA molecule comprising:
   (i) a first open reading frame encoding for a fusion protein;
   (ii) a double-stranded region comprising:
      (A) at least one target codon; and
      (B) an RNA binding motif capable of being bound by an RNA-binding domain; and
   (iii) a second open reading frame encoding for an effector protein.

207. A method for treating a cancer or microbial infection in a subject in need thereof, the method comprising administering an effective amount of:
(a) a nucleic acid encoding a fusion protein comprising an RNA-binding domain linked to a deaminase domain of an adenosine deaminase; and
(b) a nucleic acid encoding for an RNA molecule comprising:
   (i) an open reading frame encoding for an effector protein;
   (ii) a double-stranded region comprising:
      (A) at least one target codon; and
      (B) an RNA binding motif capable of being bound by an RNA-binding domain; and
   (iii) a poly-A tail.

208. The method of paragraph 207, wherein the fusion protein comprises the fusion protein of any one of paragraphs 1-82.

209. The method of paragraph 207 or 208, wherein the fusion protein comprises an RNA-binding domain linked to a deaminase domain of an adenosine deaminase.

210. The method of any one of paragraphs 207-209, wherein the fusion protein comprises:
(a) an RNA-binding domain;
(b) a first portion of a deaminase domain of an adenosine deaminase;
(c) a first member of a binding pair;
(d) a second portion of the deaminase domain; and
(e) a second member of a binding pair.

211. The method of paragraph any one of paragraphs 207-209, wherein the fusion protein comprises:
(a) an RNA-binding domain;
(b) a first portion of a deaminase domain of an adenosine deaminase;
(c) a first member of a binding pair;
(d) a second portion of the deaminase domain;
(e) a cleavable linker; and
(f) a second member of a binding pair.

212. The method of paragraph any one of paragraphs 207-211, wherein the effector protein comprises an antigen-binding domain for a cancer antigen or a microbial antigen.

213. The method of paragraph any one of paragraphs 207-212, the method further comprising administering to the subject an effective amount of:
(a) an inducer of the first and second binding pairs of the fusion protein;
(b) a cleavage inducer of the cleavable linker; and/or
(c) a protease inhibitor

214. The method of paragraph 213, wherein the inducer, cleavage inducer, or protease inhibitor is administered after the nucleic acid encoding the fusion protein and/or the nucleic acid encoding the RNA molecule.

Some embodiments of the technology described herein can be defined according to any of the following numbered paragraphs:

1. A fusion protein comprising:
(a) a first portion of a deaminase domain (DD) of an adenosine deaminase;
(b) a first member of a binding pair associated with the first portion of the DD;
(c) a second portion of the DD; and
(d) a second member of a binding pair associated with the second portion of the DD,
   wherein the first member of the binding pair is capable of binding to the second member of the binding pair in the absence of an inducer, resulting in allosteric inhibition of the first and second portions of the DD, and
   wherein the first member of the binding pair is not capable of binding to the second member of the binding pair in the presence of the inducer, resulting in activation of the first and second portions of the DD.

2. The fusion protein of paragraph 1, wherein in the allosteric inhibition of the first and second portions of the DD comprises deformation of the inositol hexaphosphate (IP6) binding pocket of first and second portions of the DD.

3. The fusion protein of paragraph 1, wherein in the activation of the first and second portions of the DD comprises deaminase activity.

4. The fusion protein of paragraph 1, further comprising an RNA-binding domain, wherein the RNA-binding domain (RBD) is capable of binding to a binding motif for the RBD on an RNA molecule.

5. The fusion protein of paragraph 4, wherein the RNA-binding domain is selected from the group consisting of MCP, PCP, λN, and HIV tat.

6. The fusion protein of paragraph 1, wherein the deaminase domain is capable of:
(a) deamination of an adenosine nucleotide into an inosine nucleotide in an RNA molecule;
(b) converting at least one stop codon into at least one non-stop codon;
(c) converting at least one start codon into at least one non-start codon;
(d) converting at least one non-start codon into at least one start codon; and/or
(d) converting at least one sense codon encoding a first amino acid into at least one mutated sense codon encoding a second amino acid.

7. The fusion protein of paragraph 1, wherein the adenosine deaminase comprises Adenosine Deaminase Acting on RNA (ADAR), Adenosine Deaminase TRNA Specific (ADAT), or Adenosine Deaminase Domain Containing (ADAD).

8. The fusion protein of paragraph 7, wherein the ADAR is ADAR1, ADAR2, or ADAR3; the ADAT is ADAT1; or the ADAD is ADAD1 or ADAD2.

9. The fusion protein of paragraph 1, wherein in the presence of the inducer, the DD is constitutively active.

10. The fusion protein of paragraph 9, wherein the constitutively active deaminase domain comprises: an E1008Q mutation in ADAR1; an E488Q mutation in ADAR2; or an E527Q mutation in ADAR3.

11. The fusion protein of paragraph 1, wherein the DD comprises at least one mutation in the IP6 binding pocket that decreases background activity.

12. The fusion protein of paragraph 11, wherein the at least one mutation is in ADAR2 in an amino acid residue selected from the group consisting of: T375, R400, R522, Y658, K662, Y668, K672, V688, K690, F697, and L699.

13. The fusion protein of paragraph 4, comprising from N-terminus to C-terminus:
(a) the RNA-binding domain;
(b) the first portion of the deaminase domain;
(c) the first member of the binding pair;
(d) the second portion of the deaminase domain; and
(e) the second member of the binding pair; or comprising from N-terminus to C-terminus
(f) the RNA-binding domain;
(g) the first member of the binding pair;
(h) the first portion of deaminase domain;
(i) the second portion of the deaminase domain; and
(j) the second member of the binding pair.

14. The fusion protein of paragraph 1, wherein the first and second portions of the deaminase domain are split at an RNA binding loop.

15. The fusion protein of paragraph 14, wherein the RNA binding loop comprises:

(a) residues G969 to K999 of ADAR1:
(SEQ ID NO: 134)
GALFDKSCSDRAMESTESRHYPVFENPKQGK of ADAR1;
(b) residues A454 to Q479 of ADAR2:
(SEQ ID NO: 135)
ARIFSPHEPILEEPADRHPNRKARGQ;
(c) residues A493 to H518 of ADAR3:
(SEQ ID NO: 136)
ARLHSPYEITTDLHSSKHLVRKFRGH;
(d) residues A334 to K365 of ADAD1:
(SEQ ID NO: 137)
AQIKSQLRLNPHSISAFEANEELCLHVAVEGK;
(e) residues A347 to Q375 of ADAD2:
(SEQ ID NO: 138)
AARDIYLPPTSEGGLPHSPPMRLQAHVLGQ;
(f) residues K974 to S986 of ADAR1:
(SEQ ID NO: 139)
KSCSDRAMES;
(g) residues F457 to D469 of ADAR2:
(SEQ ID NO: 140)
FSPHEPILEEPAD;
(h) residues P498 to S508 of ADAR3:
(SEQ ID NO: 141)
PYEITTDLHSS;
(i) residues Q339 to P344 of ADADI:
(SEQ ID NO: 142)
QLRLNP;
or
(j) residues P352 to P360 of ADAD2:
(SEQ ID NO: 143)
PPTSEGGLP.

16. The fusion protein of paragraph 14, wherein the first and second portions of the deaminase domain are split between:
(a) residues S977 and D978 of ADAR1;
(b) residues T984 and E985 of ADAR1;
(c) residues A468 and D469 of ADAR2;
(d) residues S507 and S508 of ADAR3;
(e) residues L340 and R341 of ADAD1; or
(f) residues G357 and G358 of ADAD2.

17. The fusion protein of paragraph 1, wherein the first and second members of the binding pair are:
(a) Bad and Bcl-xL, and the inducer of the first and second binding pairs is A-1331852 or ABT-737;
(b) Bim and Bcl-xL, and the inducer of the first and second binding pairs is A-1331852;
(c) MS1 and MCL-1, and the inducer of the first and second binding pairs is S63845; or
(d) a repressible protease and a protease-binding peptide, and the inducer of the first and second binding pairs is an inhibitor of the repressible protease.

18. The fusion protein of paragraph 1, wherein:
(a) the first member of the binding pair comprises an antigen-binding domain,
(b) the second member of the binding pair comprises a first antigen, and
(c) the inducer of the first and second binding pairs comprises a second antigen;
   wherein the antigen-binding domain is capable of binding to the second antigen with a similar or higher affinity than to the first antigen.

19. The fusion protein of paragraph 1, wherein the fusion protein further comprises a cleavable linker between the second portion of the deaminase domain the second member of the binding pair.

20. A fusion protein comprising:
(a) a first portion of a deaminase domain (DD) of an adenosine deaminase;
(b) a repressible protease associated with the first portion of the DD;
(c) a second portion of the DD; and
(d) a protease cleavage site associated with the first and second portions of the DD;
   wherein the repressible protease is capable of binding to the protease cleavage site in the absence of an inhibitor for the repressible protease, resulting in cleavage of the protease cleavage site and inactivation of the first and second portions of the DD; and
   wherein the repressible protease is not capable of binding to the protease cleavage site in the presence of the inhibitor for the repressible protease, resulting in activation of the first and second portions of the DD.

21. An RNA molecule comprising:
(I)
   (a) a double-stranded region comprising:
      (i) at least one target codon; and
      (ii) an RNA binding motif capable of being bound by an RNA-binding domain; and
   (b) at least one open reading frame, wherein the at least one open reading frame is operatively linked to the double-stranded region; or
(II)
   (a) a first open reading frame;
   (b) a double-stranded region comprising:
      (i) at least one target codon; and
      (ii) an RNA binding motif capable of being bound by an RNA-binding domain; and
   (c) a second open reading frame, wherein the second open reading frame is operatively linked to the double-stranded region; or
(III)
   (a) an open reading frame;
   (b) a double-stranded region comprising:
      (i) at least one target codon; and
      (ii) an RNA binding motif capable of being bound by an RNA-binding domain; and
   (c) a poly-A tail.

22. The RNA molecule of paragraph 21,
wherein the open reading frame encodes for a fusion protein or an effector protein;
wherein the first open reading frame encodes for a first polypeptide and the second open reading frame encodes for a second polypeptide;
wherein the first open reading frame encodes for a first portion of a polypeptide, and the second open reading frame encodes for a second portion of the polypeptide;
wherein the first or second open reading frame encodes for a fusion protein; or
wherein the first or second open reading frame encodes for at least one effector protein.

23. The RNA of paragraph 21, wherein the target codon is a stop codon, a start codon, a non-start codon, or a sense codon; and wherein the target codon comprises at least one adenosine nucleotide.

24. The RNA molecule of paragraph 21, wherein:
(a) the RNA-binding domain comprises MCP, and the RNA binding motif comprises MS2;
(b) the RNA-binding domain comprises PCP, and the RNA binding motif comprises PP7;
(c) the RNA-binding domain comprises λN, and the RNA binding motif comprises BoxB; or
(d) the RNA-binding domain comprises HIV Tat, and the RNA binding motif comprises TAR.

25. The RNA molecule of paragraph 21, wherein the double-stranded region of the RNA molecule comprises:
at least one hairpin.
one hairpin comprising the at least one target codon and the RNA binding motif; or
a first hairpin comprising the at least one target codon and a second hairpin comprising the RNA binding motif.

26. A system for modulating RNA translation comprising:
(a) a fusion protein comprising:
   (i) an RNA-binding domain;
   (ii) a first portion of a deaminase domain (DD) of an adenosine deaminase;
   (iii) a first member of a binding pair associated with the first portion of the DD;
   (iv) a second portion of the DD; and
   (v) a second member of a binding pair associated with the second portion of the DD; and
(b) the RNA molecule of paragraph 21.

27. The system of paragraph 26, further comprising an inducer of the first and second binding pairs.

28. The system of paragraph 26, wherein the first and second members of the binding pair of the fusion protein are capable of binding to each other in the absence of an inducer of the first and second binding pairs and reduce or prevent:
(a) the formation of the inositol hexaphosphate (IP6) binding pocket of first and second portions of the deaminase domain;
(b) deaminase activity of the first and second portions of the deaminase domain;
(c) conversion by the deaminase domain of the at least one target codon of the RNA molecule into at least one inosine-comprising codon;
(d) conversion by the deaminase domain of the at least one stop codon of the RNA molecule into at least one non-stop codon;
(e) conversion by the deaminase domain of the at least one start codon of the RNA molecule into at least one non-start codon;
(f) conversion by the deaminase domain of the at least one non-start codon of the RNA molecule into at least one start codon; and/or
(g) conversion by the deaminase domain of the at least one sense codon of the RNA molecule encoding for a first amino acid into at least one mutated codon encoding for a second amino acid.

29. The system of paragraph 26, wherein the first and second members of the binding pair of the fusion protein are not capable of binding to each other in the presence of an inducer of the first and second binding pairs, allowing for:
(a) the formation of the inositol hexaphosphate (IP6) binding pocket of first and second portions of the deaminase domain;
(b) deaminase activity of the first and second portions of the deaminase domain;
(c) conversion by the deaminase domain of the at least one target codon of the RNA molecule into at least one inosine-comprising codon;
(d) conversion by the deaminase domain of the at least one stop codon of the RNA molecule into at least one non-stop codon;
(e) conversion by the deaminase domain of the at least one start codon of the RNA molecule into at least one non-start codon;
(f) conversion by the deaminase domain of the at least one non-start codon of the RNA molecule into at least one start codon; and/or
(g) conversion by the deaminase domain of the at least one sense codon of the RNA molecule encoding for a first amino acid into at least one mutated sense codon encoding for a second amino acid.

30. The system of paragraph 29, wherein conversion by the deaminase domain of at least one target codon in the RNA molecule into at least one inosine-comprising codon increases or decreases:
(a) translation of the reading frame of the RNA molecule;
(b) translation of the second reading frame of the RNA molecule;
(c) degradation of the RNA molecule; and/or
(d) translation of a variant polypeptide encoded by the RNA molecule.

The technology described herein is further illustrated by the following examples which in no way should be construed as being further limiting.

### EXAMPLES

### Example 1

### Brief Summary of the Embodiment

Nucleic acid based medicines, including messenger RNA (mRNA) based vaccines and therapeutics have emerged as a promising technology with many applications in both medicine and basic science research. Instead of producing and delivering a protein directly to cells/organisms/patients, nucleic acids (including mRNAs) are delivered to cells via lipid nanoparticles (LNP) or other agents. Upon entry, ribosome mediated-translation results in the production of proteins encoded by the delivered nucleic acid sequences. A limitation of mRNA-based agents is that uptake of the mRNA to any human cell type will result in its translation and thus expression of the encoded protein. Thus, a limitation of mRNA based medicines is the limited control over translation of an encoded protein sequence. To overcome this limitation, described herein is a set of technologies that permit programmable control over the translation state and stability of mRNAs in a way that can be regulated via exogenous inducers (e.g., drugs, light, etc.) based on presence of intracellular signals and components (including proteins, enzymes, antigens, and cell-type markers, etc.). The technology described herein relates to compositions, systems and methods that permit control over mRNA modification and subsequent translational decoding (e.g., protein expression) through stop-codon editing and read-through of an mRNA transcript. RNA editing can be mediated by an engineered human adenosine deaminase acting on RNA (ADAR) enzyme domains possessing engineered conditional activity, which can be regulated via exposure to drugs or light, or activated in response to post-translational modifications (e.g., proteolysis or through the binding of a specified target proteins/antigens of interest.

ADAR proteins catalyze the deamination of adenosine into inosine, and this modification is among the most prevalent post-transcriptional modifications in mammalian cells. ADAR proteins are conserved across phyla, with humans containing three homologues, two of which are catalytically active (ADAR1 & ADAR2). ADAR can be used to edit UAG stop codons to UIG in the presence of a complimentary strand of RNA to form dsRNA in trans and cis. Inosine's hydrogen bonding profile closely resembles guanine, and a UIG codon typically base-pairs with the tryptophan tRNA anticodon, allowing for the incorporation of tryptophan instead of the recruitment of release factors, and translation continues. ADAR can also be used to edit endogenous transcripts, correct deleterious mutations, and to turn on protein expression upon hybridization of an endogenous transcript to synthetic transcripts.

The current technology utilizes a site near the RNA binding loop on hADAR2 and hADAR1 deaminase domains that can accommodate insertions of small protein domains and/or peptides without substantially affecting editing activity. Fusion of a protein domain or peptide on the C-terminus of the deaminase domain that forms a hetero/homodimer to its cognate partner in the RNA binding loop greatly reduces the editing activity of ADAR. The use of protein-peptide interaction partners and/or cleavable linkers that can be disassociated and/or separated upon drug, light, protease, or ligand addition restores deaminase activity of the enzymatic domain. By complexing and localizing this engineered deaminase downstream of an editable stop codon, a user-defined input leads to the editing of the stop codon and readthrough to the protein cargo. Conversely, if the editable stop codon is the final stop codon before the mRNA polyA tail, then readthrough leads to the recruitment of endogenous Skip7, the initiation of non-stop decay, and the destruction of both the protein via the host proteasome and the mRNA via host exonucleases. The implementation of stop-codon editing to turn on protein expression or lead to mRNA turnover can be used in tandem in single-transcript circuits to lead to multi-input or complex logic, including but not limited to rapid induction with signal A (e.g., drug, light, protease, or ligand) and rapid mRNA decay via signal B (e.g., if the editable stop codon is the final stop codon before the mRNA polyA tail).

Exemplary applications include, but are not limited to, the development of dose-able and time-resolved mRNA therapeutics. In this scheme, mRNA can be delivered to a patient, and a physician can control the timing, degree and duration of protein expression by using a drug. Additionally, this technology can be applied to make therapeutics that produce protein only in certain cells that have a specific protein expression profile (e.g., virally infected cells, senescent cells, or cancerous cells). In this scheme, the inactive ADAR can be activated only in response to the binding of a specific protein partner or upon specific protease activity. This application reduces off-target effects if a user is looking to apply protein expression to only a certain subset of cells. Finally, this technology can be used for the development of sensitive in-vitro sensors and/or diagnostics through the coupling of ADAR-editing activity to cell-free translation systems.

Without wishing to be bound by theory, the following variations of the system are contemplated herein.

Alternative manufacturing methods can be used.

As described herein, some embodiments use DNA-encoded elements and transient transfection with plasmid DNA, which permits the complexing of purified ADAR protein with in vitro transcribed RNA. Exemplary applications include RNA vaccines and therapeutics. As such, in some embodiments, the compositions and methods described herein can be performed with bases used in mRNA vaccines to evade innate immune response, such as N1-methyl-pseudouridine containing transcripts.

In some embodiments, the ADAR protein can be produced in the absence of inositol hexaphosphate (IP6), which is a required cofactor for RNA editing by ADAR. Thus, pre-complexing of RNA and ADAR in the absence of IP6 prior to delivery to a subject ensures that ADAR cannot edit the RNA. The ADAR can gain activity only when administered to a subject either by adding IP6 to the complex prior to administration to the subject or relying on host IP6 to serve as ADAR's co-factor.

In some embodiments, the ADAR-RNA system can be used in an in-vitro/cell-free context as a sensor. In this context, the enzymatic activity of ADAR upon addition of a stimulus of interest can be coupled to cell-free translation of a reporter gene on the RNA to get sensitive detection of the stimulus.

Alternative chemistry can be used.

As described herein, the readout can be the translation of a downstream protein or the turnover of a synthetic transcript. An alternative readout that can be useful in pure biological contexts or in sensing applications is the actual base that is edited (e.g., adenine turning to hypoxanthine). This change can be detected by cDNA sequencing or fluorescence in situ hybridization (FISH) assays as inosine preferentially base pairs with cytosine and can be read out that way. In this context, bulk RNAseq can be coupled to ADAR based sensing to produce a readout of a population of cells.

As described herein, the constrained state of the ADAR by self-binding domains can be relieved by competitive binding or cleavage of a linker; however, an alternative method can be done by physical/chemical deformation of the binding/chemical interface by post-translational modifications. In this method, one of the binding components also has sites for post-translational modifications (e.g., phosphorylation by kinases, ubiquitination by ubiquitin ligases, methylation, etc.) that interfere with the intramolecular binding affinity. In this case the activity of a host enzyme interferes directly with the binding interface between the two autoinhibitory components and leads to relaxation of the constrained ADAR.

Another variation of the work described above is the use of non-genetically encoded components. Everything described above utilized genetically encoded ADAR variants relying on cellular synthesis of the repressed ADAR-DDs. However, it is useful to instead use other chemistry to link the RBL to the C-terminus and use other chemical means to open such a linkage. As an example, the two parts of the protein can be linked together in-vitro with a linker that contains azobenzene, dialkoxydiphenylsilane, or other chemically, bio-orthogonally cleavable linkers. Additionally, the use of other chemistries can be useful for the photo-dissociative/photocleavable component of the patent, as there are a host of photocleavable linkers which can be inserted between the domains in vitro.

Another variation that was tested was inserting tandem ligand-binding protein pairs in tandem in the 5′ RNA binding loop. Bcl-xL and BAD peptide insertions were tested at that site, and it was found that the resulting ADAR-DD was drug-inducible in a manner analogous, though lower extent of editing was found, to the C-terminal fusion of Bcl-xL and BAD insertion at the 5′ RNA binding loop. Other variations tested were inserting PhoCl and AsLOV2 domains in the 5′ RNA binding loop. Other variations include inserting tandem antibody-antigen pairs and other chemically or light disrupted protein interaction pairs.

### Alternative Constructs:

ADAR-DDs orthologs from other organisms can be considered as well as the paralogous ADAR1 protein from humans. There is a high degree of conserved sequence identity between these two. Without wishing to be bound by theory, it was hypothesized that the same mechanism and homologous sites can function similarly to ADAR2-DD. Secondly, additional work was done utilizing different BH3 and Bcl-2 family domains and different point mutants of these peptides. Third, NS3 inhibitor (e.g., grazoprevir, etc.) inducible ADAR2-DD construct were created using NS3/4A and NS3 binding peptide as the autoinhibitory domains.

It is contemplated herein that any antibody-fragment and antigen pairing can be used in the iADAR construct. There are many examples of antibody-antigen, ligand-receptor, and ligand-binding protein interactions (therapeutic and otherwise) known in the art that can be utilized. Additionally, proteolytic activation can use the synthetic TEV protease, an HIV protease, HCV protease, SARS-CoV2 protease, or other protease-inducible version (also referred to herein as repressible proteases).

On the synthetic transcript side, fluorescent protein reporters can be substituted with any protein of interest upstream (e.g., the ADAR itself, a sensing component of the ADAR domain, etc.) and/or the fluorescent protein reporters downstream of the editable stop codon can be substituted with any protein of interest (e.g., suicide genes such as caspases for a kill switch, different cytokines for inflammatory or anti-inflammatory effects, etc.).

One example is encoding an ADAR-DD with a chemically-inducible dimerization domain (CID) next to its repressive domain and encoding upstream on the RNA the same binding partner fused to the other CID domain. When drug is added, the repressed ADAR-DD-CID fusion colocalizes with the second CID-peptide component that is soluble and expressed in the cell, and the increased local concentration leads to trans binding and activation of ADAR.

Additionally, the specific RNA sequences used in the RNA hairpin, RNA binding element, and 3′UTR/polyA tail are only exemplary, and minor or major changes in the primary, secondary, or tertiary structure of these RNA elements can be done.

Another variation is the modulation of C-terminal linker length by exogenous addition. In this scenario, there is a certain linker length between the ADAR-DD and the dimerization domain whereby the ADAR-DD is spontaneously active. By placing domains that functionally change radius with stimuli (e.g., LOV domain J alpha helix with light or HCV NS3-NS3-binding peptide with drug) then the ADAR can be turned from an inactive to an active state. Another variation to make a light or protease inducible ADAR2 can be by inducing the availability of a cryptic protease site (e.g., hiding a cut site in a LOV domain).

### Additional Alternatives:

The constructs described herein can be administered using virus-mediated delivery.

The constructs described herein can be used to detect viral antigens in infected cells, including the expression of viral proteases.

Instead of alternating translation or transcript stability, modifications can be used to alter different aspects of regulation, including trafficking or subcellular localization.

The constructs described herein can be used combined with self-replication mechanisms to mediate amplification within specific cell types.

### Example 2

### Technical Description for Iadar-Dd Editing

Adenosine deaminase acting on RNA (ADAR) enzymes are conserved across phyla and are responsible for the conversion of adenosine to inosine in eukaryotic messenger RNA (mRNA), a common and critical post-transcriptional modification. Inosine has a different hydrogen bonding pairing than adenine, and although it is capable of base pairing with cytosine, uracil, and adenine, it has been shown to preferentially base pair with cytosine. This change in base-pairing preference allows for a phenomenon called recoding, where a codon that previously encoded one amino-acid/release-factor is changed to base-pair to a different codon during translation. Previous groups have utilized ADAR editing of the amber stop codon UAG to UIG to allow for read-through and downstream translation of a protein of interest from a synthetic transcript.

As disclosed herein, the technology described herein relates to protein engineering of adenosine deaminase (AD), e.g., human ADAR deaminase domains (DD), such that the ADAR is in the constitutively inactive state. This engineering of the DDs of the ADAR enables the ADAR to be inducible, e.g., it is an engineered inducible ADAR (iADAR). For iADAR to become activated i.e., turned ON, it is allosterically modulated from an inactive to an active state in response to small molecule drugs, target antigen-binding, protease activity, and light. As disclosed herein, in one embodiment, pairing the engineered iADAR with a synthetic mRNA transcript that localize the iADAR to an editable stop codon allows for enzyme activity to be coupled to a change in synthetic, user-defined protein expression and/or mRNA stability. Therefore, this invention enables the selective editing of synthetic mRNA transcripts based on user defined and potentially endogenous inputs.

Applicants disclosed herein an exemplary proof of principal synthetic constructs are generated, which are referred to herein as Target Activation Constructs (TAC) or Target inactivation constructs (TIC), that demonstrate and validate as exemplary mRNA transcripts that can be edited by iADAR in the active state (iADAR-ON). The inventors first tested whether they could edit stop codons in a synthetic mRNA transcript with a constitutively active ADAR2-DD(E488Q) fused to the C-terminus of bacteriophage-derived MS2 coat protein (MCP), a protein which binds a specific RNA motif Previous groups have demonstrated the editing activity of this protein construct on dsRNA duplex between a substrate strand and a guide strand. However, the inventors were interested in testing whether by using a short hairpin motif, they could get stop codon editing on the same strand that they directed the DD to when expressed in human cells.

The inventors demonstrate use of a first Target Activation construct (TAC), which is a synthetic transcript that contains 4 parts (FIG. 1A): an upstream coding region (red), a short hairpin that contains 1 or more stop codons (black), an RBD binding motif (gray), and a downstream coding region (green). For the reporter system, the inventors used the fluorescent protein-epitope tag tandem fusions of mCherry-FLAG and HA-mNeonGreen as the upstream and downstream coding regions. Without DD editing activity, translation is terminated after mCherry-FLAG. Coexpression with MCP-ADAR2-DD was hypothesized to lead to binding of MCP-DD, stop codon UAG editing to UIG, and expression of mNeonGreen (FIG. 1B).

HEK 293FT cells were co-transfected with plasmids expressing the reporter containing 2 UAG stop codons in the hairpin and a pcDNA3 construct encoding a fusion protein composed of MCP, ADAR2-DD(E488Q), and TagBFP. As a control, they also co-transfected a catalytically inactive DD containing the mutation E396A. Two days after transfection, fluorescence microscopy showed that mNeonGreen robustly expressed in the population co-transfected with active ADAR-DD whereas there was minimal mNeonGreen fluorescence in the catalytically inactive condition (FIG. 1C). They further confirmed this result by performing a Western blot on HEK293FT cells co-transfected with MCP-ADAR-DD and reporter constructs containing different numbers of stop codons and RNA elements (FIG. 1D). The Western blot showed HA-mNeonGreen expression in non-trivial, control conditions only when MCP-ADAR and a reporter containing an MS2 operator were co-transfected. Almost no off-target editing was observed in stop-codon reporters with the downstream MS2 operator, demonstrating that the ADAR is relatively specific to transcripts containing an RNA encoded RBD binding motif.

The inventors sought to test the modularity of the RNA-binding-domain (RBD) and RNA-elements by swapping out the MCP and MS2 operator with other RBDs and RNA elements: PP7 coat protein (PCP) and PP7 operator, HIV Tat peptide and trans-activation response element (TAR), and λ bacteriophage N peptide (λN) and BoxB operator. mNeonGreen expression was strongly observed by fluorescent microscopy when the correct RBD and RNA element were co-transfected into HEK293FT cells, and the mNeonGreen-to-mCherry ratio is similar across RBD-element pairs demonstrating similar relative editing efficiencies (FIG. 1E).

In addition to testing a synthetic transcript which utilizes stop-codon editing to turn on a downstream protein product, which has been previously demonstrated, the inventors sought to create an original way to couple enzymatic ADAR activity to protein and mRNA degradation: an OFF-switch. In order to do so, the inventors designed a synthetic transcript (FIG. 2A) where there is an open reading frame (green), 1 or more stop codons in an RNA-hairpin (dark gray), a specific RNA element (light gray), and a 3′UTR with no downstream stop codons prior to the polyadenylate (polyA) tail. The inventors created a reporter plasmid that contained a bidirectional CMV driving the expression dTomato on one transcript and EGFPd2, an editable stop codon, an MS2 operator, and a mutated rabbit-β-globin 3′UTR/polyA signal sequence lacking additional stop codons on the other transcript (FIG. 2B). In the absence of ADAR-DD activity, both dTomato and EGFPd2 should be translated and expressed. However, coexpression of MCP-ADAR2 should lead to editing of the only in-frame stop codon and the initiation of an endogenous process called non-stop decay, whereby the translating ribosome stalls on the polyA-tail and recruits Ski7 followed by exonucleases to destroy the mRNA (FIG. 2C). In this process, proteosomal degradation of the nascent, unreleased polypeptide also occurs.

In order to test this, HEK 293FT cells were co-transfected with plasmids encoding the biCMV reporter with 2 UAG stop codons in the hairpin and a pcDNA3 construct encoding an MCP fusion to catalytically active and inactive ADAR2-DD (FIG. 2D). Analysis by fluorescence microscopy 48-hours post-transfection demonstrated that coexpression of the catalytically active DD leads to greatly decreased EGFPd2 expression levels relative to the catalytically inactive DD. This is the first example of ADAR's being used to downregulate protein translation and lead to mRNA degradation via non-stop decay.

The previous section demonstrates the ability of ADAR activity to lead to increased or decreased protein expression and/or mRNA stability, but all of this work was carried out using a constitutively active ADAR-DD. The second of this work is the engineering of ADAR-DD's to be allosterically regulated by small-molecule drugs, competitive antigen binding, protease, and light.

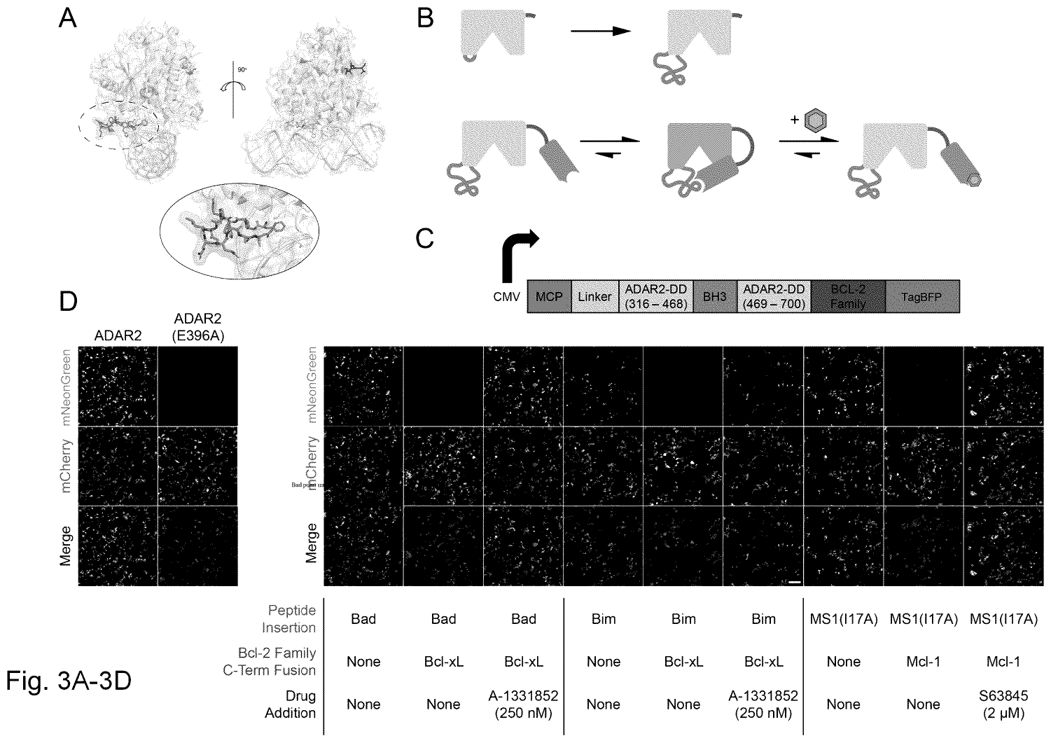

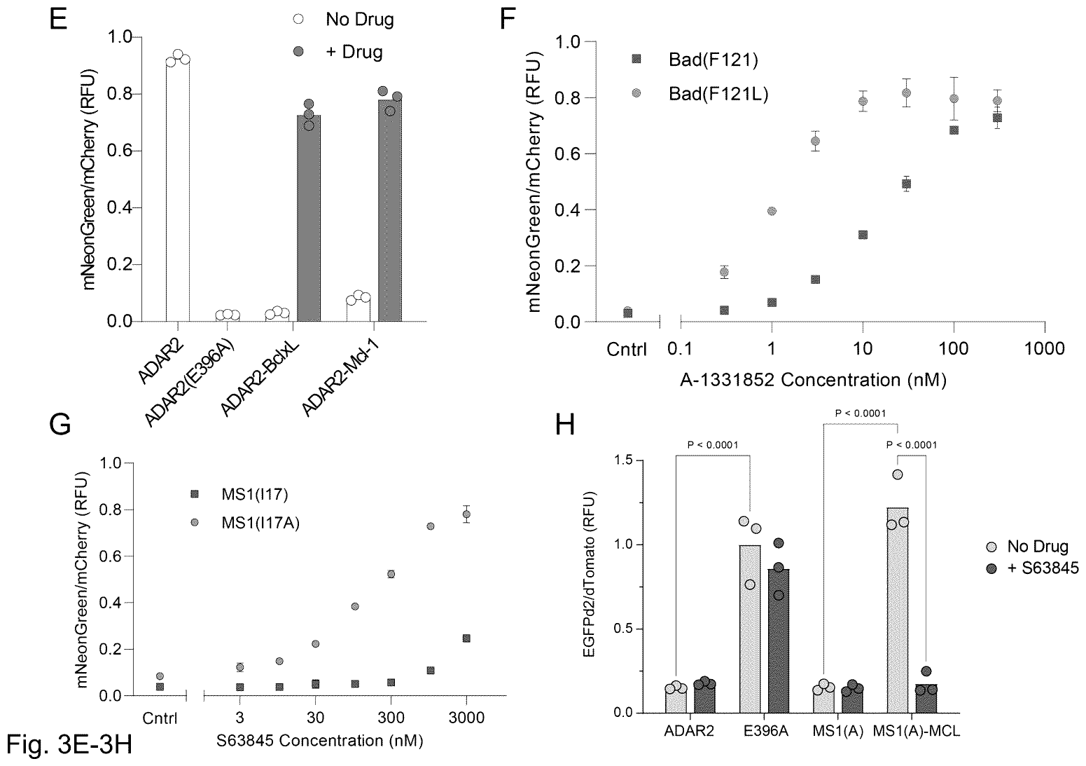

In order to accomplish this, the inventors investigated whether sites on the 5′ RNA binding loop (RBL) of human ADAR2-DD between residues F457 and D469 (green in FIG. 3A) can accommodate peptide/protein insertions without serious decreases in catalytic activity. Previous work has demonstrated that this loop is non-conserved between paralogs (with ADAR1 having a large insertion at this cite relative to ADAR2), that the residue identity in this loop can be mutated, and that splitting ADAR2 at this position results in halves that have modest activity when reconstituted. Inspired by work on engineered drug-inducible enzymes via terminal or internal fusion of drug-dissociative protein domains, the inventors sought to insert a peptide/protein into the 5′ loop (FIG. 3B top) that can reversibly bind to a peptide/protein partner at the C-terminus (FIG. 3B bottom). The addition of a small-molecule drug that competitively binds the same site as the inserted peptide/protein shifts the equilibrium away from the bound, repressed conformation and towards the open, catalytically active conformation (FIG. 3B bot).

As a demonstration of this principle, the inventors designed and constructed ADAR2-DD variants with B-cell lymphoma-2 (Bcl-2) homology-3 (BH3) proteins, which are peptides which bind reversibly to Bcl-2 family proteins, inserted into the RBL (FIG. 3C). The inventors also fused cognate Bcl-2 family binding partners to the C-terminus to create a reversible, autoinhibited ADAR-DD. As BH3-Bcl-2 family interactions regulate apoptosis and are dysregulated in various diseases including cancer, there are a host of small molecule drugs which can ablate BH3-Bcl2 binding. The inventors constructed ADAR2-DD variants with only the BH3 peptides Bad and Bim inserted at the RBL between A469 and D470 (with a 7 amino acid linker between A469 and the start of the BH3 peptides), and autoinhibited variants with Bad and Bim insertions and human Bcl-xL fused to the C-Terminus of ADAR2-DD. The inventors also constructed a DD variant with a synthetic, mutated Mcl-1 binding peptide MS1(I17A), and a variant with MS1(I17A) and human Mcl-1 (AA 171-326) fused to the RBL and C-terminus respectively.

To investigate whether these ADAR variants were functional and drug-responsive, the inventors co-transfected HEK293FT with the mCherry-mNeonGreen reporter construct and the MCP-ADAR-DD variants described above with inhibitors added at the time of transfection (250 nM of A-1331852 for Bcl-xL and 2 μM of S63845 for Mcl-1 containing proteins). Two days later, fluorescence microscopy images were taken of the cell populations. The inventors saw that BH3 peptide insertions on their own did not greatly affect the enzymatic activity of ADAR2 (FIG. 3D), that C-terminal fusion of Bcl-2 binding partners greatly inhibited ADAR editing activity, and that treatment with drug was able to rescue editing activity. The inventors repeated the experiment and analyzed the samples by flow-cytometry. The relative fluorescence of mNeonGreen to mCherry in transfected cells mirrored the microscopy results and showed that the inventors were able to successfully create allosteric and drug-responsive ADAR2 variants with a similar dynamic range to native and catalytically inactive ADAR2-DD by fusing chemically disruptable heterodimeric protein partners at the RBL and C-terminus (FIG. 3E).

The inventors investigated whether the allosteric ADAR-DD activity was dose responsive by performing a titration with A-1331852 for the ADAR-Bad-BclxL variant and with a variant containing a single point mutation (F121L) (FIG. 3F). The point mutant variant was more drug responsive that the original Bad-peptide variant, and activated at concentrations as low as 1 nM and maxed out at ˜30 nM of A-1331852. The inventors also performed a titration of S63845 on the ADAR-MS1(A)-Mcl-1 variant and compared it to an ADAR variant with the original MS1 peptide, and found that the MS1(A) variant is far more drug-responsive than the original MS1 peptide, with mNeonGreen activation beginning to be seen at 100 nM (FIG. 3G).

As a final test, the inventors used the MS1(I17A)/Mcl-1 variant with the dTomato-BiCMV-EGFPd2 reporter plasmid (FIG. 2B), which should lose EGFPd2 fluorescence with ADAR editing of the final stop codon. The inventors transfected HEK293FT cells with both constructs, added 2 μM of S63845 at the time of transfection, and 48 hours later assessed relative fluorescent levels of transfected cells by flow cytometry, taking the median of EGFPd2/dTomato for that population of cells (FIG. 3H). The inventors found that the MS1(I17A) peptide insertion and the drug treated MS1(I17A)/Mcl-1 ADAR-DDs reduced EGFPd2/dTomato fluorescence levels to similar extents as original ADAR-DD, and that the MS1(I17A)/Mcl-1 variant without drug had similar levels as the catalytically inactive variant. The inventors therefore demonstrated a general mechanism to create a drug-inducible ADAR-DD that can function in human cells and in a variety of different contexts.

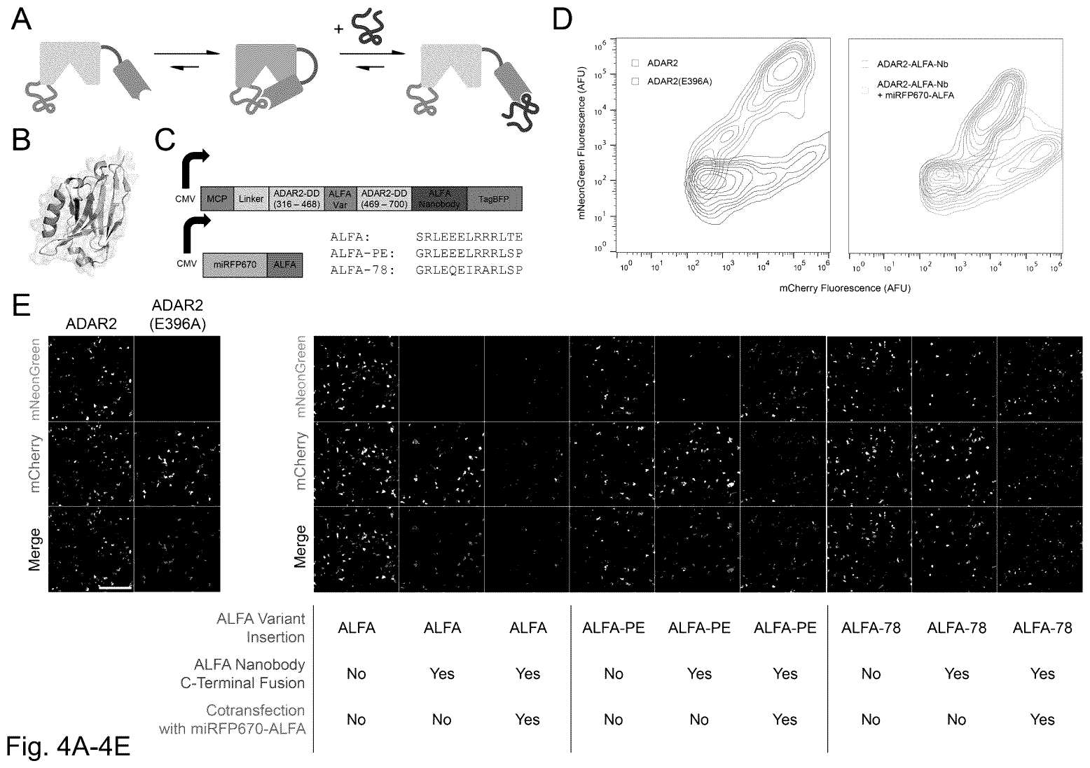

The inventors further sought to investigate whether this same allosteric mechanism of competitive-binding leading to the dissociation of a repressive intramolecular interaction between RBL and C-terminal domains could be applied more generally to make an ADAR that is activated by binding to any epitope (FIG. 4A). In this system, a peptide/protein/epitope insertion at the RBL (green) and an antibody fragment (gray) fused to the C-terminus (red) interact and inhibit ADAR2 enzyme activity (blue). However, the addition of another antigen that can bind the same antibody fragment (purple) leads to the dissociation of the inhibitory intramolecular interaction and to ADAR-DD activity. This would be a broadly useful development and create a sensing mechanism that can enable the specific targeting of mRNA translation to cell types with a specific proteomic profile (e.g., oncogenes or viral proteins).

Seeking a model antigen-antibody fragment pair, the inventors used the ALFA epitope tag and anti-ALFA nanobody (NbALFA) due to it being a well-characterized interaction pair that works in the cytosol of living cells and has a number of described, weaker variants (FIG. 4B). The inventors constructed ADAR-DDs with three variants of the ALFA epitope tag of differing nanobody binding affinities inserted between residues A469 and D470 (ALFA, ALFA-PE, & ALFA-78) (FIG. 4C). The inventors also constructed ADAR-DD variants with the NbALFA fused to the C-terminus (FIG. 4C), which should lead to an intramolecular, inhibitory interaction between the ALFA and NbALFA that can be relieved by the binding of an ALFA tag fused to a second construct (FIG. 4A).

The inventors transiently co-transfected HEK 293FT cells with the mCherry-mNeonGreen reporter, MCP-ADAR2-DD variants containing different configurations of ALFA-tag insertions and NbALFA fusions, and uncaging constructs containing miRFP670 fused to ALFA. Without wishing to be bound by theory, it was hypothesized that the intramolecular interaction would inhibit the ADAR2-DD, but that the ALFA tag on the miRFP670 would displace the lower strength ALFA variants (PE and 78), and perhaps at high enough concentrations displace the intramolecular ALFA, and lead to increased editing.

The inventors analyzed the cells by flow cytometry (FIG. 4D) and by microscopy (FIG. 4E) after 48 hours. The ADAR-DD's with ALFA variants inserted were all active and lead to mNeonGreen expression when alone (FIG. 4E), and the fusion of the NbALFA to the C-terminus decreased mNeonGreen fluorescence in all ALFA variants though more strongly according to the interaction affinity (ALFA>ALFA-PE>ALFA-78). The co-transfection with miRFP670-ALFA lead to elevated mNeonGreen expression levels in all cases, even with ADAR-ALFA-Nb where the competitive antigen is the same as the intramolecular antigen (FIGS. 4D and E). The lower affinity ALFA-PE and ALFA-78 variants however showed higher editing rates when co-transfected when compared to ALFA, which was expected. This clearly demonstrates an allosteric ADAR2-DD that is activated by binding of a user defined antigen.

The final component of the constructs detailed here is an ADAR-DD that can be activated by proteolytic and/or photolytic cleavage of the linker between the C-terminus of ADAR-DD and the N-terminus of the fused intramolecular binding domain. The inventors previously found that Bcl-xL fusion to ADAR2-DD with Bad inserted at the RBL was only inactive with a C-terminal fusion, and that soluble co-expression or N-terminal fusion did not lead to an auto-inhibited ADAR2-DD (data not shown). This implied that Bcl-xL binding to the Bad peptide at the RBL was not by itself inhibitory, but that its fusion to the C-terminus was causing the protein to adopt an inhibited state/conformation. Without wishing to be bound by theory, it was hypothesized that as opposed to inducing an active conformation by competitive binding, the inventors could induce an active ADAR-DD state by changing whether one component is fused/associated to the C-terminus of the DD.

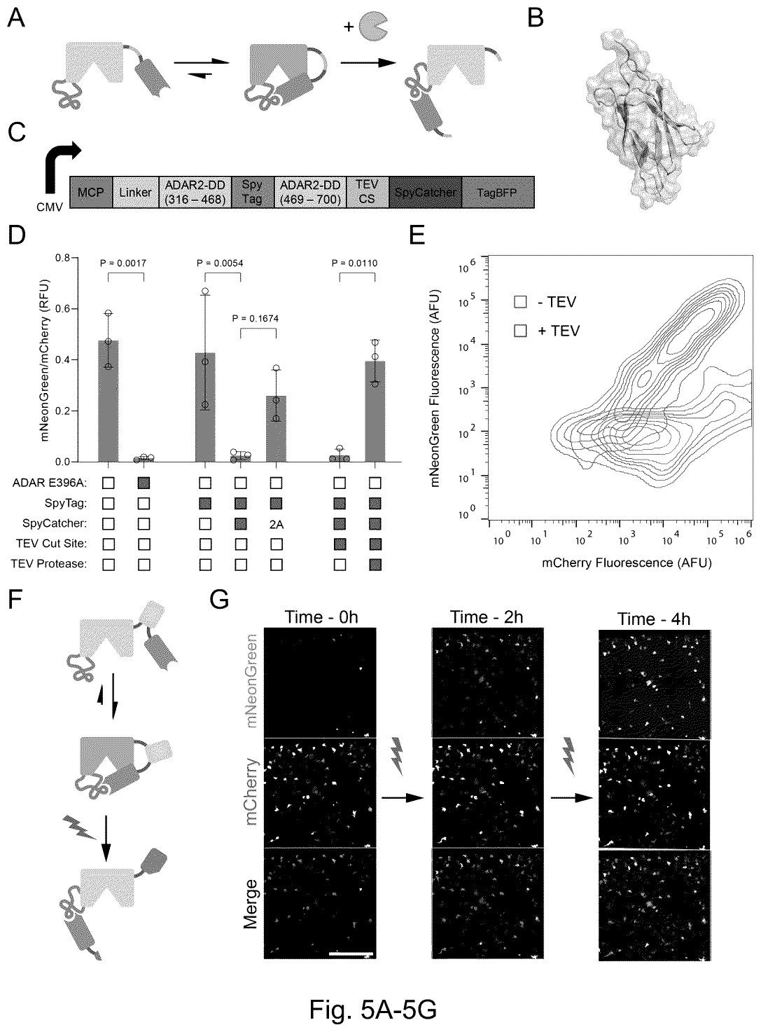

The first method that the inventors tested was activation through proteolytic cleavage. In this scheme (FIG. 5A), a protein/peptide is inserted at the RBL, its protein/peptide binding partner is fused to the C-terminus of ADAR-DD, and a protease cut site (polypeptide sequence prone to proteolytic cleavage) is inserted between ADAR-DD and the C-terminal fusion. When a protease that can cleave the specific cut site is present, the cleavage relieves the inhibitory conformation. For this scheme, the inventors used the engineered SpyTag and SpyCatcher which forms a covalent isopeptide bond between the domains (FIG. 5B), making the interaction irreversible. The inventors constructed an MCP-ADAR-DD variant that contained SpyTag inserted at the RBL, a variant containing SpyTag and SpyCatcher fused to the C-terminus, and a construct that co-expressed ADAR-SpyTag and SpyCatcher separated by the “self-cleaving-peptides” P2A and T2A. Without wishing to be bound by theory, it was hypothesized that the SpyTag-SpyCatcher interaction would only inhibit ADAR activity when the SpyCatcher was fused to the C-terminus and not when separated from the ADAR-DD (T2A construct).

When cotransfected with the mCherry-mNeonGreen reporter, activity was seen in the SpyTag variant and with the T2A-SpyCatcher variant, but not with the C-Terminal fusion, confirming that association alone did not inhibit ADAR2-DD (FIG. 5D). The inventors then made an ADAR-DD variant with an inserted TEV protease cut site (amino acids ENLYFQG, SEQ ID NO: 286) between the C-terminus of ADAR2-DD and the N-terminus of SpyCatcher (FIG. 5C). The inventors co-transfected HEK 293FT cells with the reporter construct, the ADAR-DD TEVcs variant, and the TEV protease and 48 hours later analyzed the transfected cells via flow cytometry (FIG. 5D-5E). The ADAR-DD variant with SpyTag at the RBL and a TEVcs separating the DD and SpyCatcher was only active with the addition of TEV protease, and its induced activity was close to that of the non-engineered ADAR2-DD.

This demonstrates that the technology can be adapted to make other ADAR variants that are responsive to other proteases, as only the cut-site in the linker needs to change. This technology can be applied to make a system that turns on translation of a therapeutic protein in response to viral or other disease-associated proteases, or can be paired with other systems to make sensitive protease sensors.

With proteolytic-based induction demonstrated, the inventors sought to create an ADAR-DD variant that could instead undergo photocleavage induction. In order to achieve this, the inventors constructed an ADAR-DD variant using Bad inserted at the RBL, PhoCl—an engineered photocleavable protein based on a circularly permuted mMaple fluorescent protein—fused to the C-terminus of ADAR-DD, and Bcl-xL fused to the C-terminus of PhoCl. In this configuration (FIG. 5F), PhoCl serves as a linker between the C-terminus of ADAR and the N-terminus of a protein/peptide binding domain that can be cleaved by violet (˜405 nm) light.

This ADAR-DD variant was fused to MCP and was subsequently co-transfected into HEK 293FT cells with the mCherry-mNeonGreen reporter plasmid. 48 hours after transfection, the media was replaced with OptiMEM and the sample was moved to an epifluorescent microscope to be imaged and tracked over time. Subsequent to the first image being captured, the sample was illuminated with violet light for 10 s using an HXP 120V light source with an EBFP filter. Two hours later, fluorescent images were recorded, and the same sample underwent another 10 s of violet light illumination. A final image was captured two hours after this event (four hours after the first illumination event). The inventors observed that mNeonGreen fluorescence greatly increased post-illumination relative to mCherry fluorescence, which remained relatively constant, implying that the photocleavage of the PhoCl linker lead to the production of a more active ADAR2-DD (FIG. 5G).

### Example 3

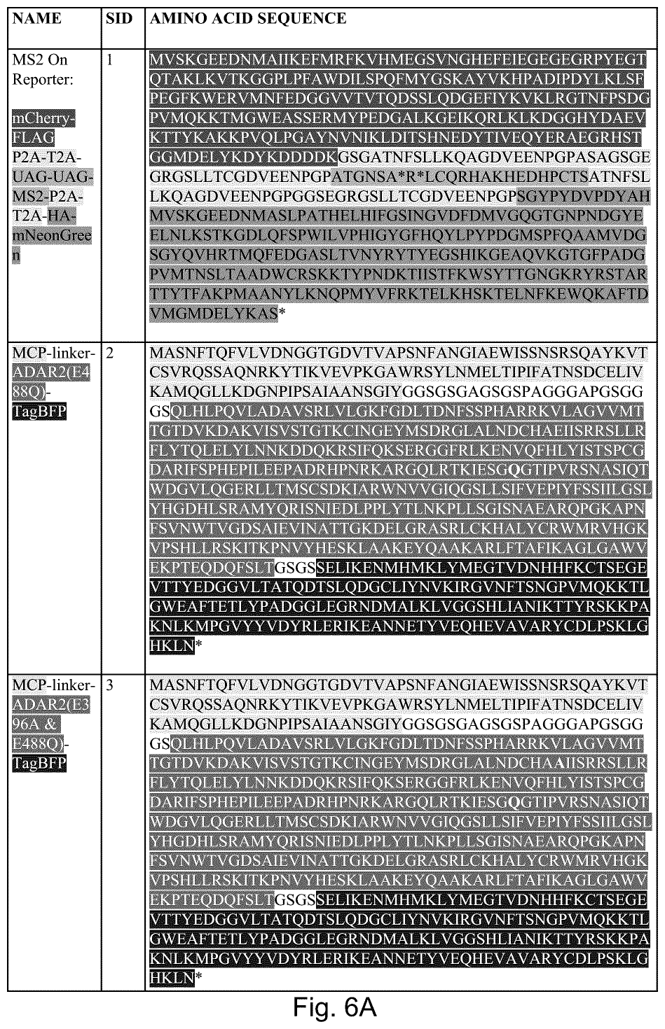

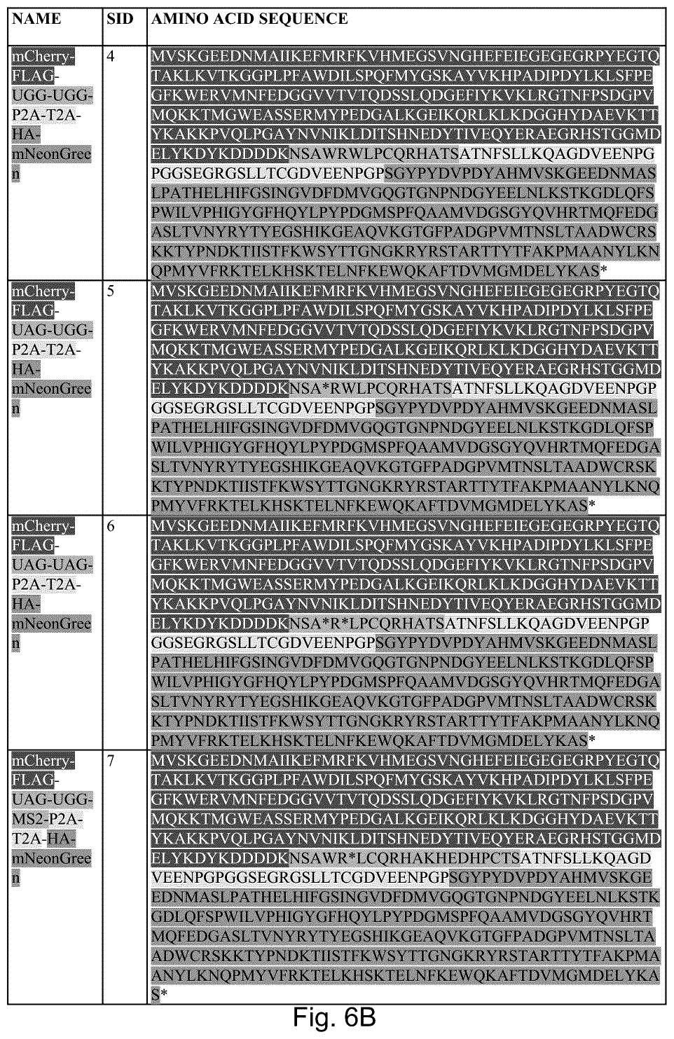

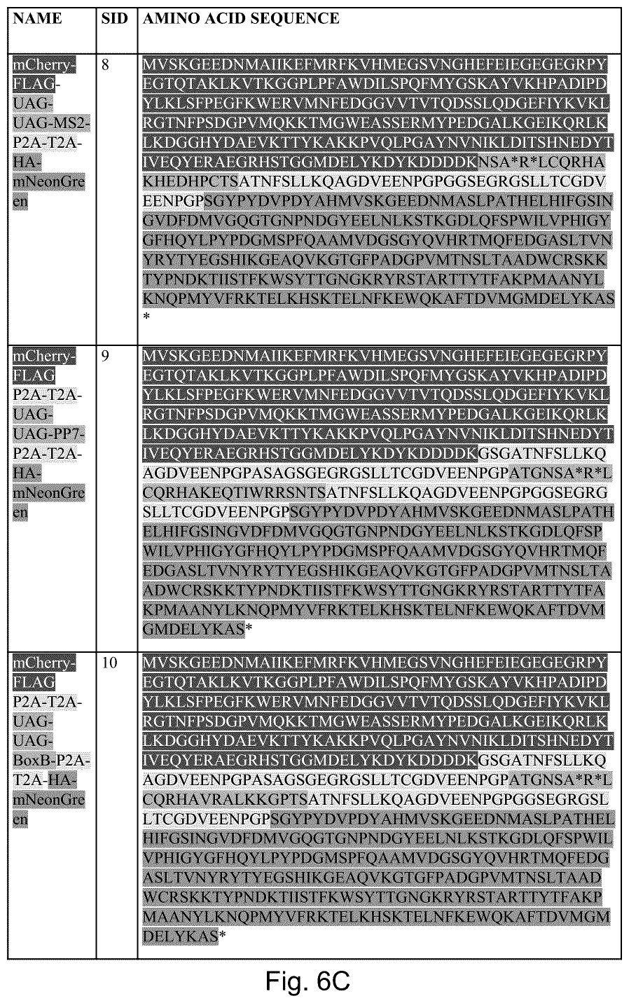

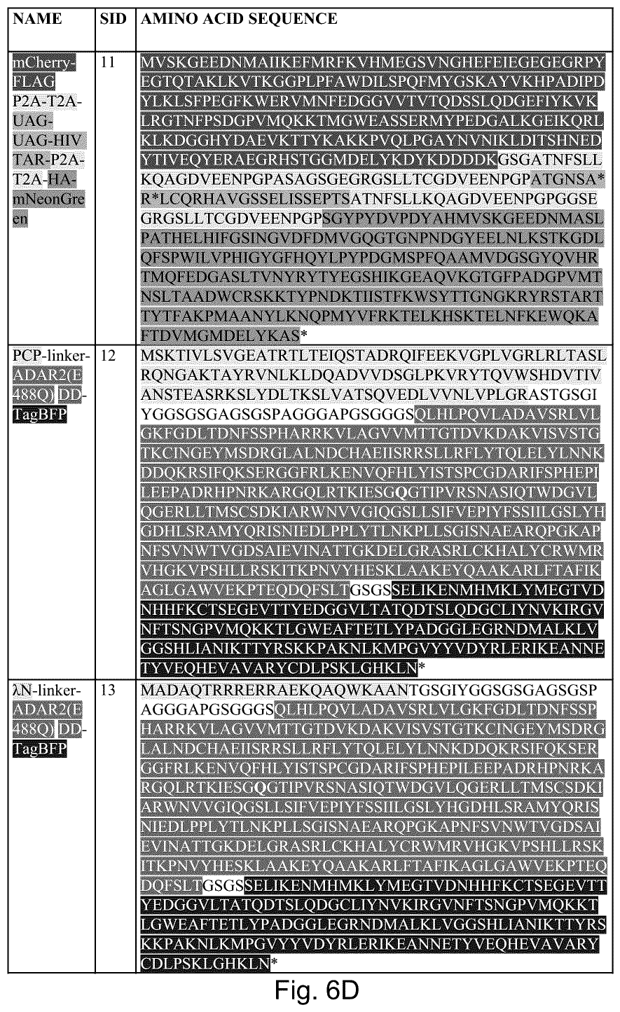

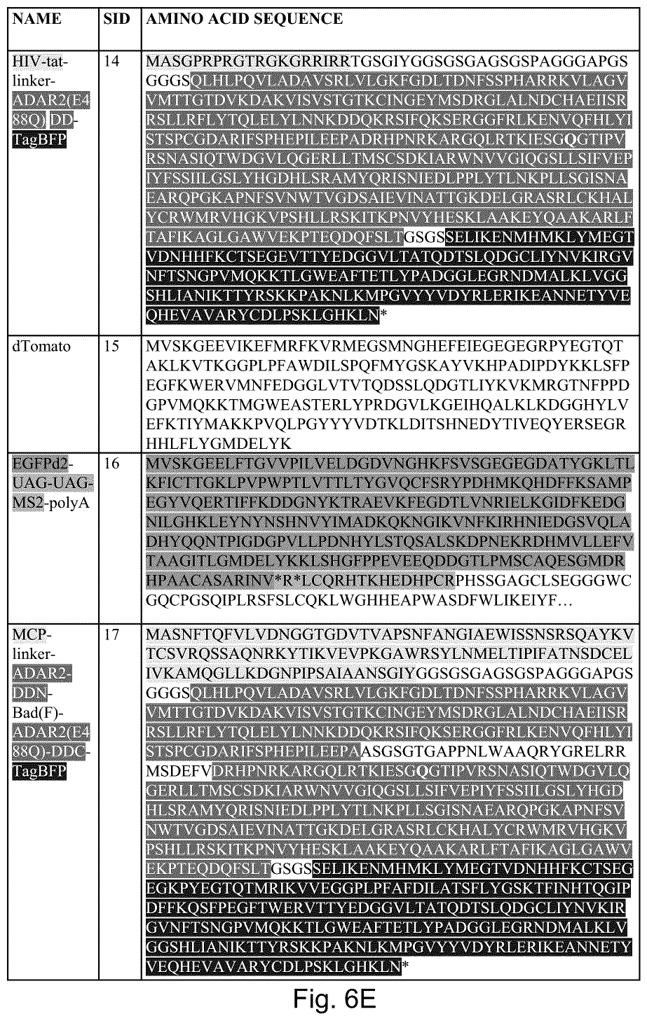

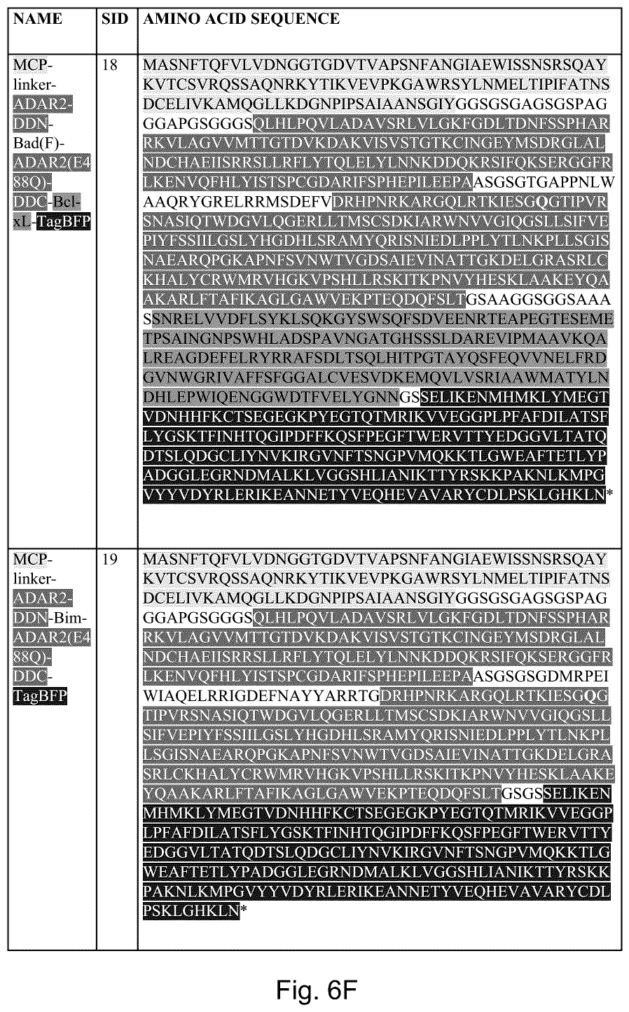

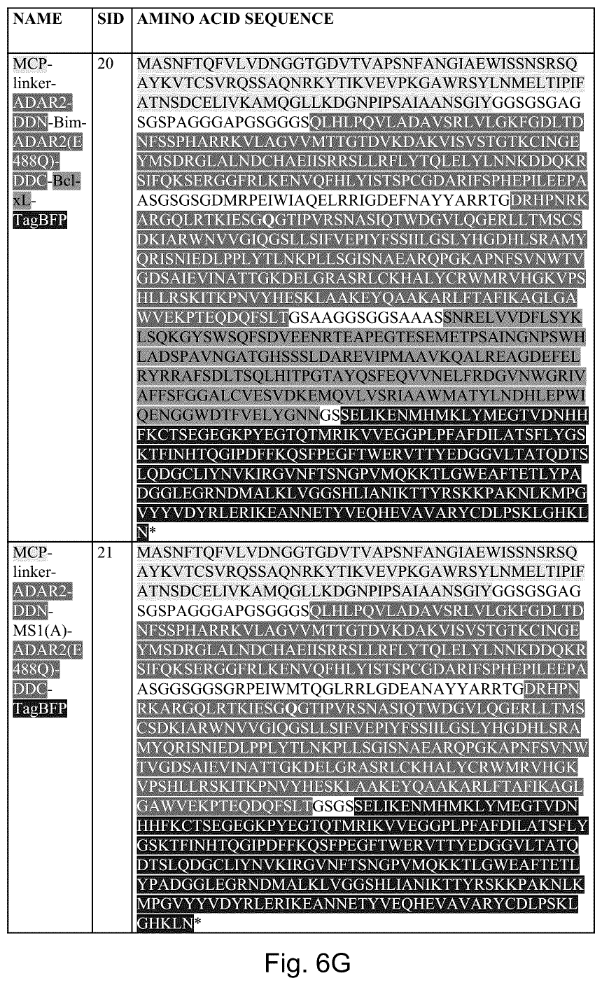

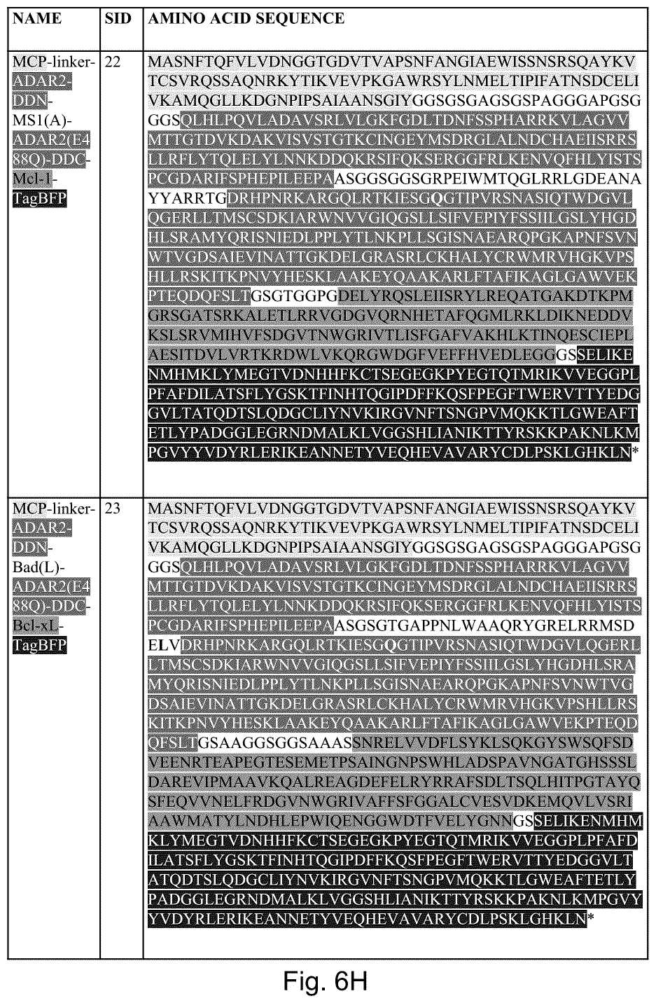

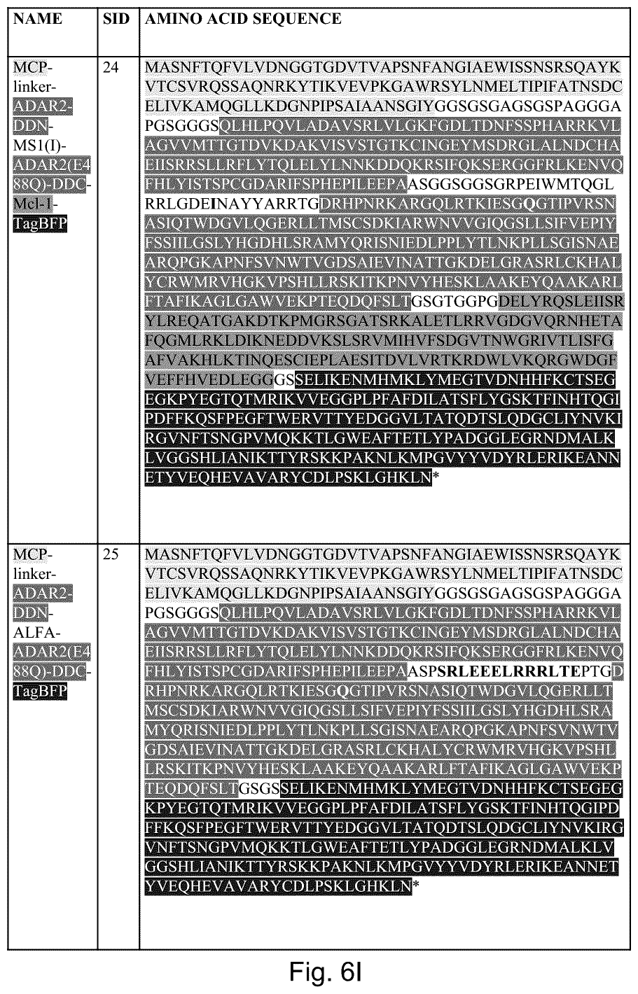

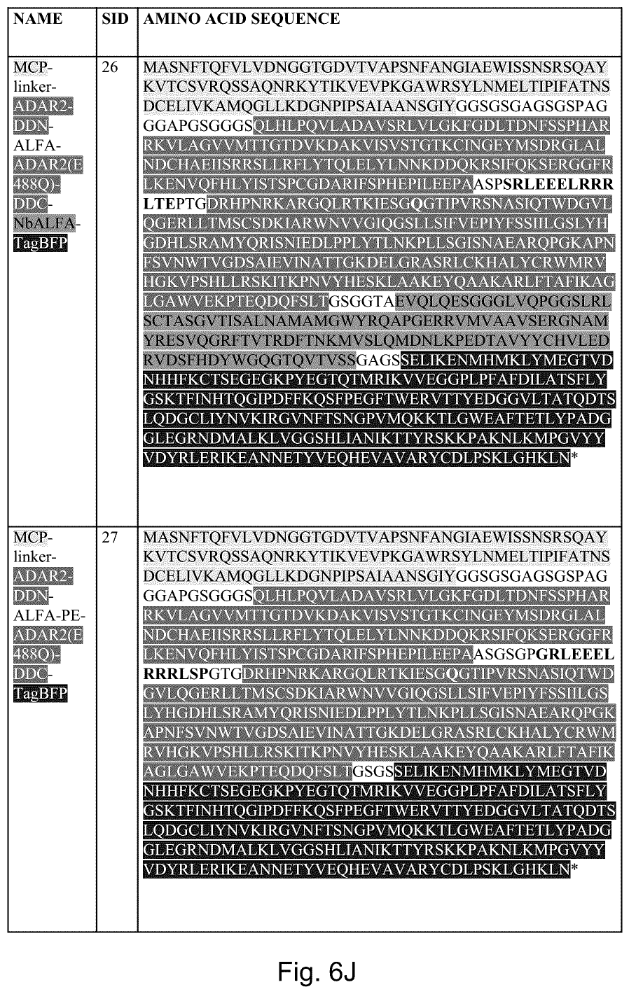

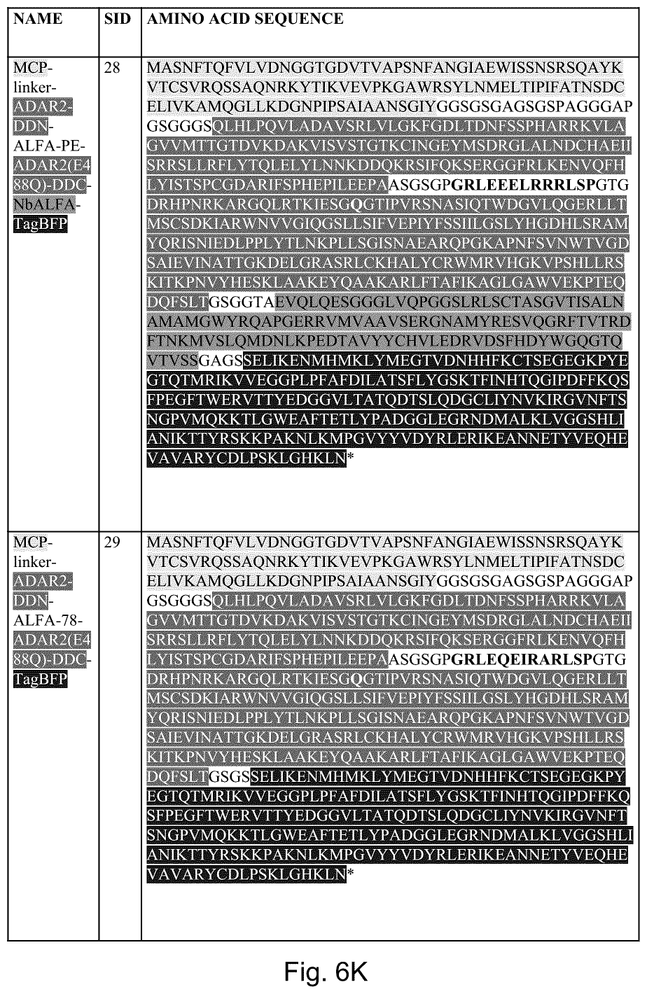

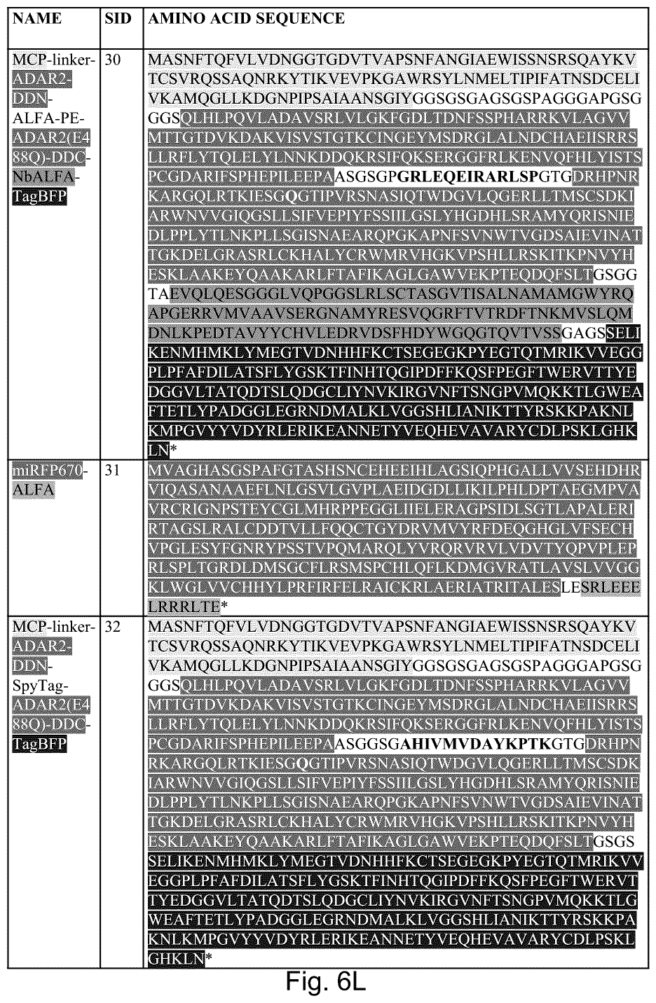

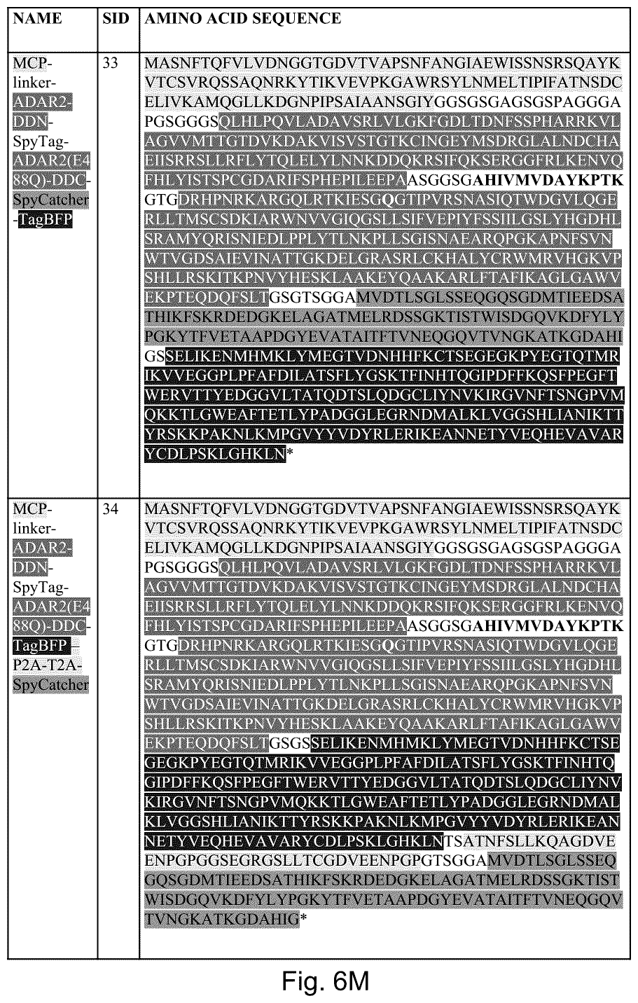

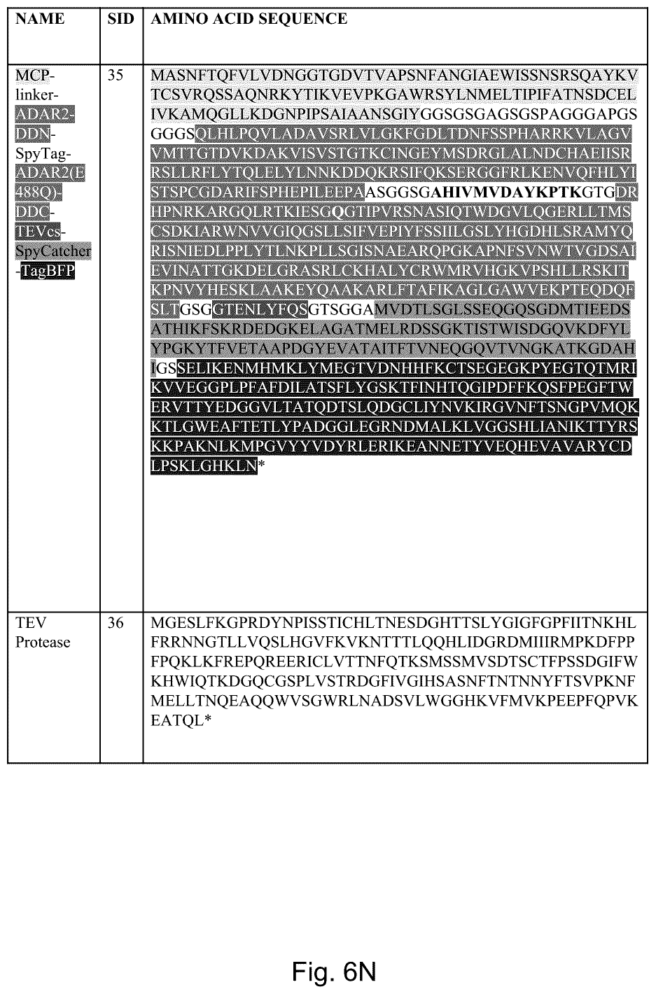

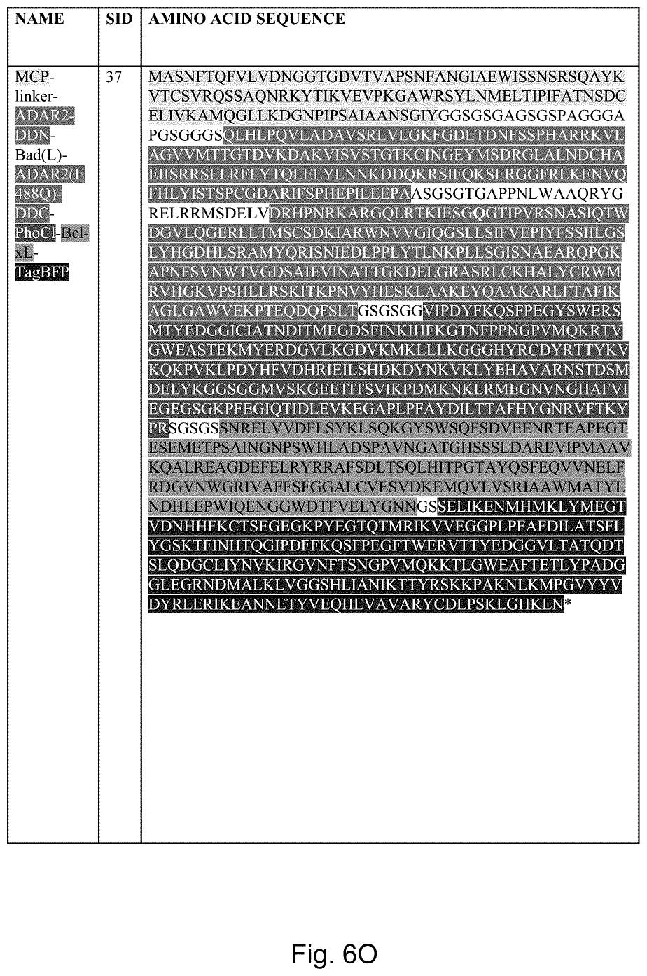

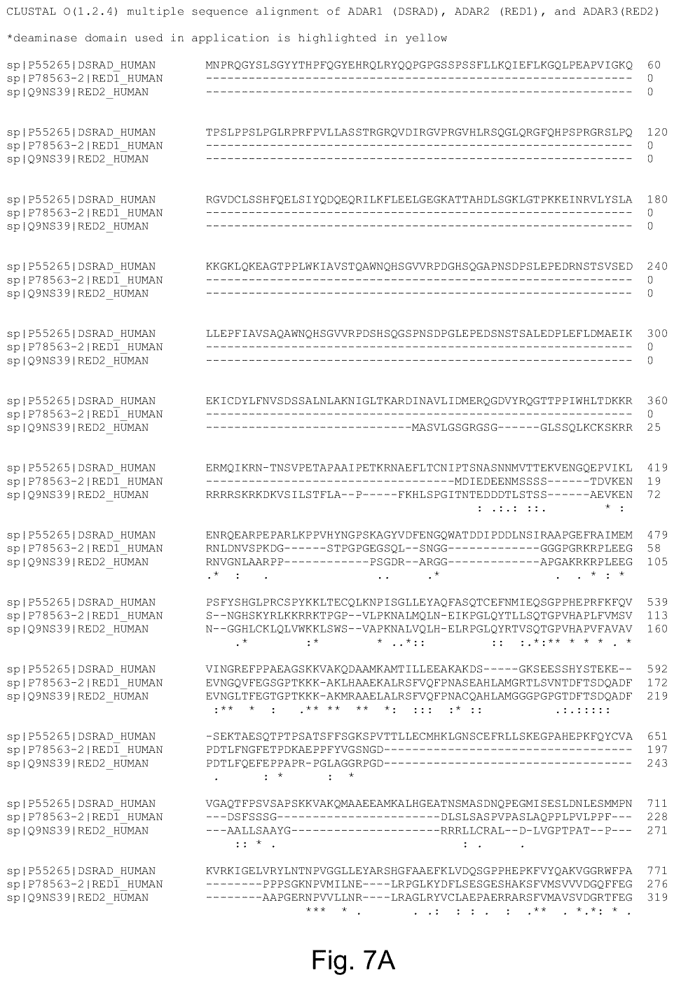

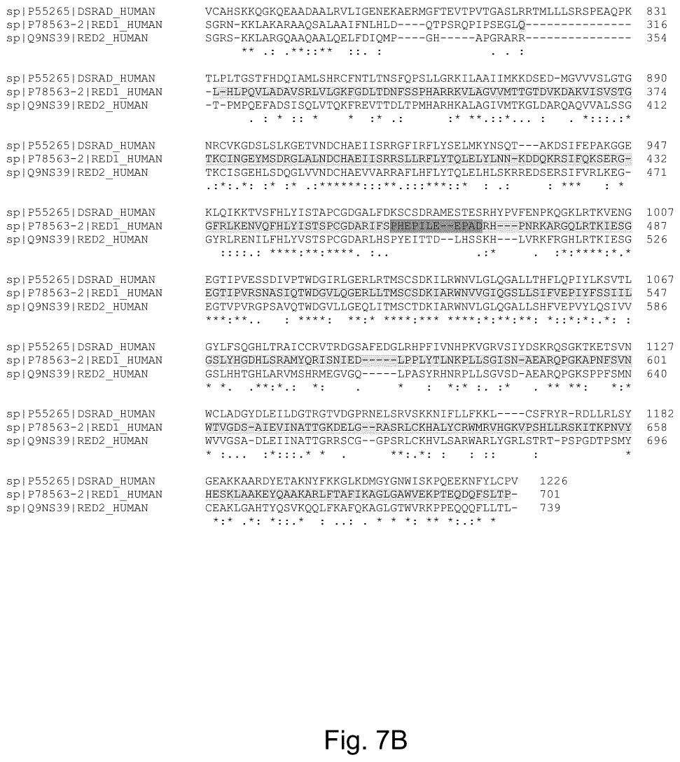

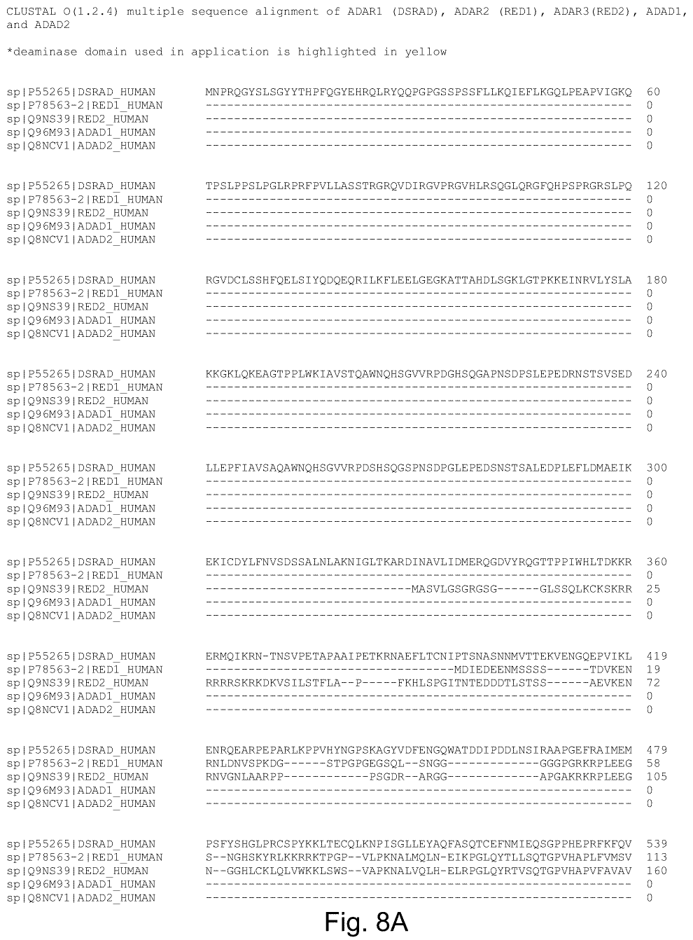

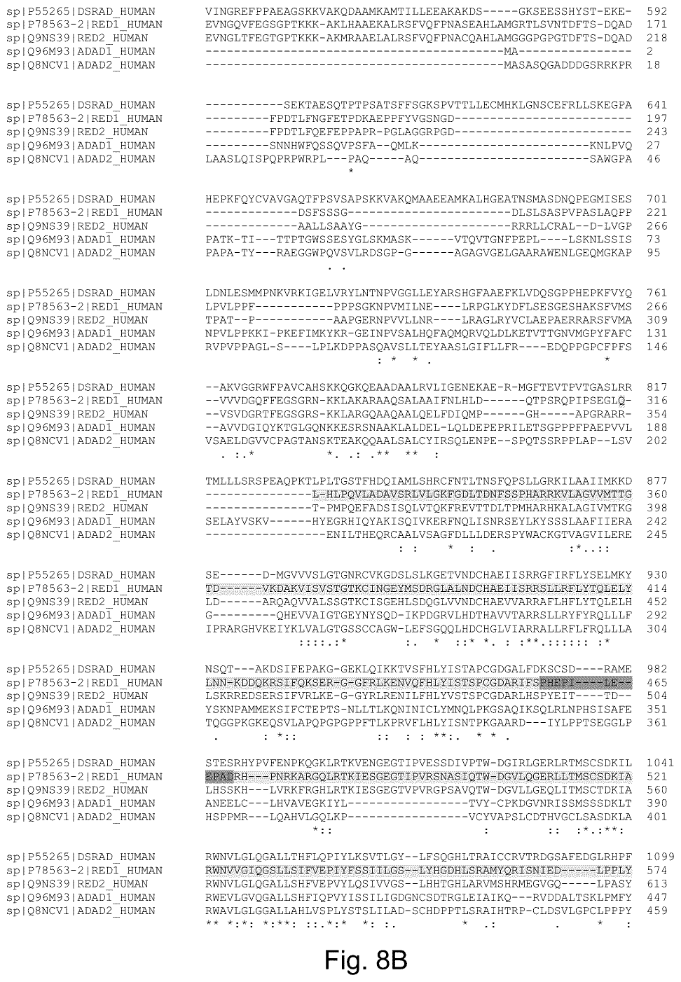

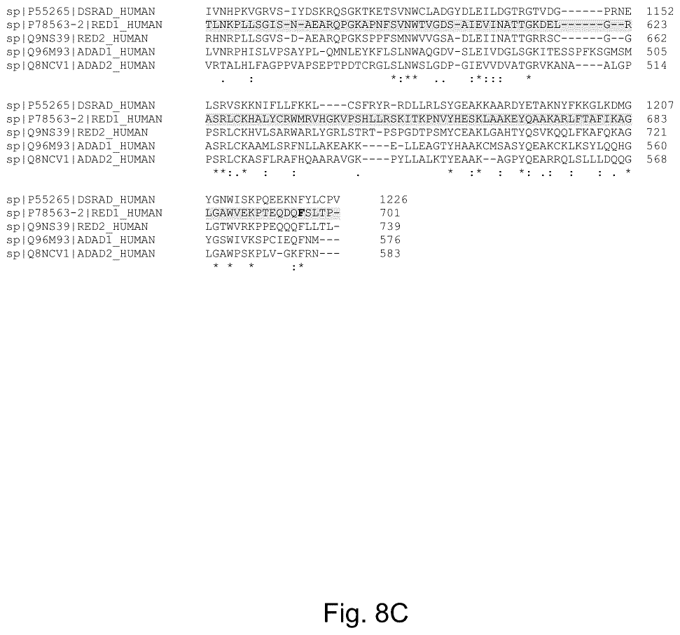

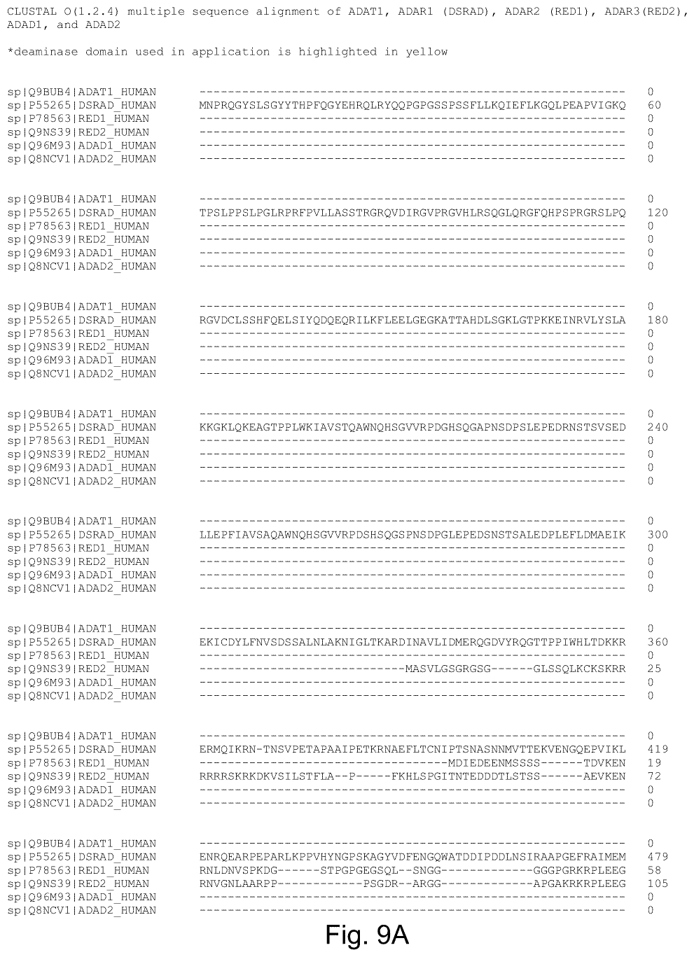

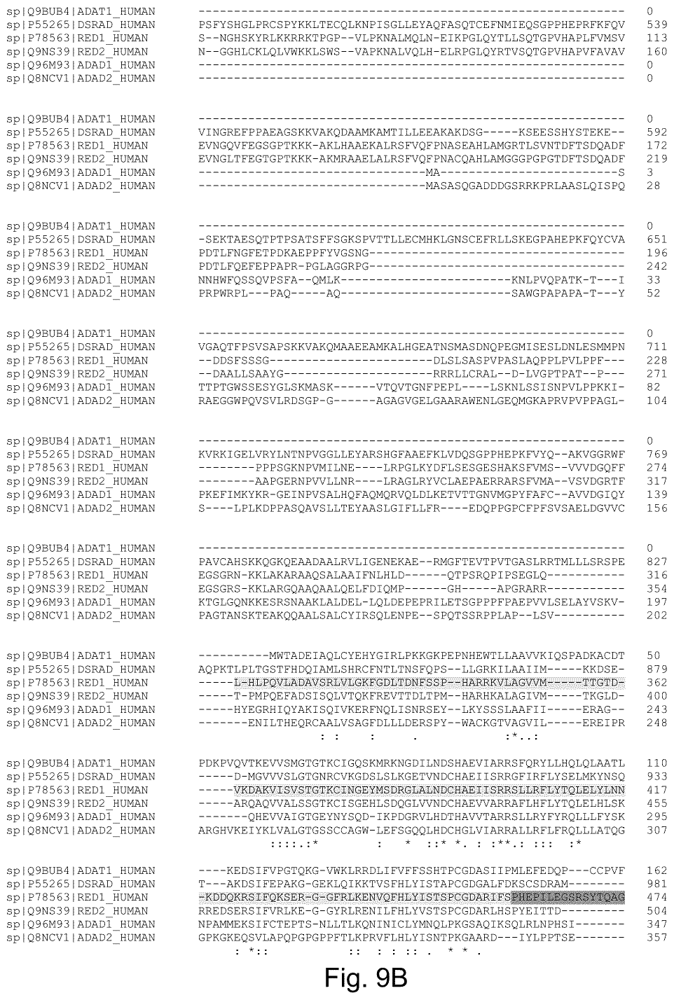

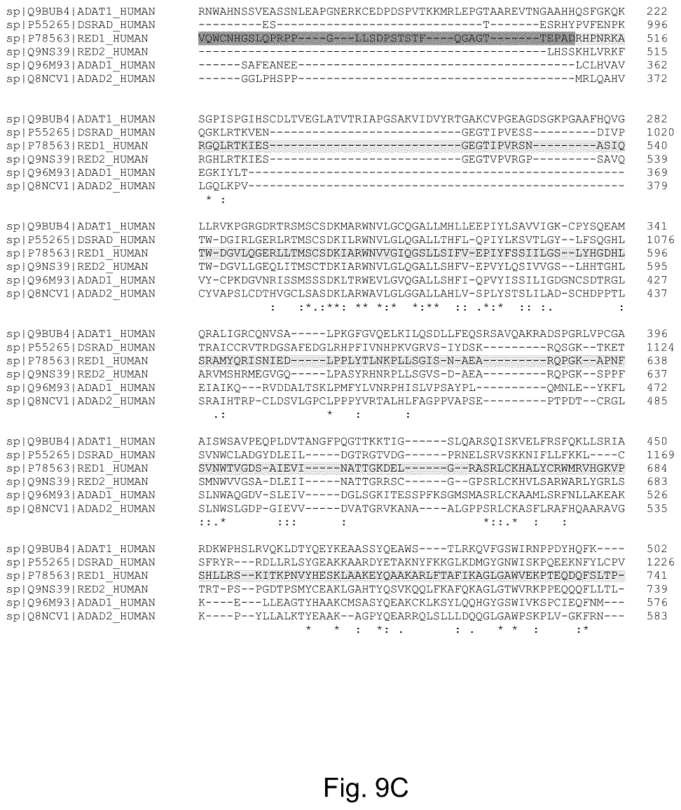

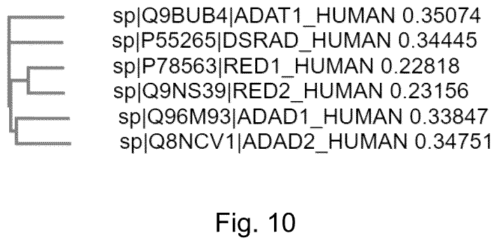

TABLE 1
Amino Acid Sequence Table (see e.g., FIG. 6)

| | SEQ | | |
|---|---|---|---|
| | ID | See e.g., | |
| NAME | NO | FIGS. | AMINO ACID SEQUENCE |
| MS2 On | 1 & | 1C, 1E, | MVSKGEEDNMAIIKEFMRFKVHMEGSVNGHEFEIEGEGE |
| Reporter: | 104 | 3D-G, | GRPYEGTQTAKLKVTKGGPLPFAWDILSPQFMYGSKAYV |
| mCherry- | | 4D-E, | KHPADIPDYLKLSFPEGFKWERVMNFEDGGVVTVTQDSS |
| FLAG | | 5D-E, 5G | LQDGEFIYKVKLRGTNFPSDGPVMQKKTMGWEASSERM |
| P2A-T2A- | | | YPEDGALKGEIKQRLKLKDGGHYDAEVKTTYKAKKPVQ |
| UAG-UAG- | | | LPGAYNVNIKLDITSHNEDYTIVEQYERAEGRHSTGGMD |
| MS2-P2A- | | | ELYKDYKDDDDKGSGATNFSLLKQAGDVEENPGPASAG |
| T2A-HA- | | | SGEGRGSLLTCGDVEENPGPATGNSA*R*LCQRHAKHED |
| mNeonGreen | | | HPCTSATNFSLLKQAGDVEENPGPGGSEGRGSLLTCGDV |
| | | | EENPGPSGYPYDVPDYAHMVSKGEEDNMASLPATHELHI |
| | | | FGSINGVDFDMVGQGTGNPNDGYEELNLKSTKGDLQFSP |
| | | | WILVPHIGYGFHQYLPYPDGMSPFQAAMVDGSGYQVHR |
| | | | TMQFEDGASLTVNYRYTYEGSHIKGEAQVKGTGFPADGP |
| | | | VMTNSLTAADWCRSKKTYPNDKTIISTFKWSYTTGNGKR |
| | | | YRSTARTTYTFAKPMAANYLKNQPMYVFRKTELKHSKT |
| | | | ELNFKEWQKAFTDVMGMDELYKAS* |
| MCP-linker- | 2 | 1C-E, | MASNFTQFVLVDNGGTGDVTVAPSNFANGIAEWISSNSR |
| ADAR2(E4 | | 2D | SQAYKVTCSVRQSSAQNRKYTIKVEVPKGAWRSYLNME |
| 88Q)- | | 3D-H, | LTIPIFATNSDCELIVKAMQGLLKDGNPIPSAIAANSGIYG |
| TagBFP | | 4D-E, | GSGSGAGSGSPAGGGAPGSGGGSQLHLPQVLADAVSRLV |
| | | 5D-E | LGKFGDLTDNFSSPHARRKVLAGVVMTTGTDVKDAKVI |
| | | | SVSTGTKCINGEYMSDRGLALNDCHAEIISRRSLLRFLYT |
| | | | QLELYLNNKDDQKRSIFQKSERGGFRLKENVQFHLYISTS |
| | | | PCGDARIFSPHEPILEEPADRHPNRKARGQLRTKIESGQGT |
| | | | IPVRSNASIQTWDGVLQGERLLTMSCSDKIARWNVVGIQ |
| | | | GSLLSIFVEPIYFSSIILGSLYHGDHLSRAMYQRISNIEDLPP |
| | | | LYTLNKPLLSGISNAEARQPGKAPNFSVNWTVGDSAIEVI |
| | | | NATTGKDELGRASRLCKHALYCRWMRVHGKVPSHLLRS |
| | | | KITKPNVYHESKLAAKEYQAAKARLFTAFIKAGLGAWVE |
| | | | KPTEQDQFSLTGSGSSELIKENMHMKLYMEGTVDNHHFK |
| | | | CTSEGEVTTYEDGGVLTATQDTSLQDGCLIYNVKIRGVN |
| | | | FTSNGPVMQKKTLGWEAFTETLYPADGGLEGRNDMALK |
| | | | LVGGSHLIANIKTTYRSKKPAKNLKMPGVYYVDYRLERI |
| | | | KEANNETYVEQHEVAVARYCDLPSKLGHKLN* |
| MCP-linker- | 3 | 1C-E, | MASNFTQFVLVDNGGTGDVTVAPSNFANGIAEWISSNSR |
| ADAR2(E3 | | 2D, | SQAYKVTCSVRQSSAQNRKYTIKVEVPKGAWRSYLNME |
| 96A & | | 3D-H, | LTIPIFATNSDCELIVKAMQGLLKDGNPIPSAIAANSGIYG |
| E488Q)- | | 4D-E, | GSGSGAGSGSPAGGGAPGSGGGSQLHLPQVLADAVSRLV |
| TagBFP | | 5D-E | LGKFGDLTDNFSSPHARRKVLAGVVMTTGTDVKDAKVI |
| | | | SVSTGTKCINGEYMSDRGLALNDCHAAIISRRSLLRFLYT |
| | | | QLELYLNNKDDQKRSIFQKSERGGFRLKENVQFHLYISTS |
| | | | PCGDARIFSPHEPILEEPADRHPNRKARGQLRTKIESGQGT |
| | | | IPVRSNASIQTWDGVLQGERLLTMSCSDKIARWNVVGIQ |
| | | | GSLLSIFVEPIYFSSIILGSLYHGDHLSRAMYQRISNIEDLPP |
| | | | LYTLNKPLLSGISNAEARQPGKAPNFSVNWTVGDSAIEVI |
| | | | NATTGKDELGRASRLCKHALYCRWMRVHGKVPSHLLRS |
| | | | KITKPNVYHESKLAAKEYQAAKARLFTAFIKAGLGAWVE |
| | | | KPTEQDQFSLTGSGSSELIKENMHMKLYMEGTVDNHHFK |
| | | | CTSEGEVTTYEDGGVLTATQDTSLQDGCLIYNVKIRGVN |
| | | | FTSNGPVMQKKTLGWEAFTETLYPADGGLEGRNDMALK |
| | | | LVGGSHLIANIKTTYRSKKPAKNLKMPGVYYVDYRLERI |
| | | | KEANNETYVEQHEVAVARYCDLPSKLGHKLN* |
| mCherry- | 4 | 1D | MVSKGEEDNMAIIKEFMRFKVHMEGSVNGHEFEIEGEGE |
| FLAG- | | | GRPYEGTQTAKLKVTKGGPLPFAWDILSPQFMYGSKAYV |
| UGG-UGG- | | | KHPADIPDYLKLSFPEGFKWERVMNFEDGGVVTVTQDSS |
| P2A-T2A- | | | LQDGEFIYKVKLRGTNFPSDGPVMQKKTMGWEASSERM |
| HA- | | | YPEDGALKGEIKQRLKLKDGGHYDAEVKTTYKAKKPVQ |
| mNeonGreen | | | LPGAYNVNIKLDITSHNEDYTIVEQYERAEGRHSTGGMD |
| | | | ELYKDYKDDDDKNSAWRWLPCQRHATSATNFSLLKQA |
| | | | GDVEENPGPGGSEGRGSLLTCGDVEENPGPSGYPYDVPD |
| | | | YAHMVSKGEEDNMASLPATHELHIFGSINGVDFDMVGQ |
| | | | GTGNPNDGYEELNLKSTKGDLQFSPWILVPHIGYGFHQY |
| | | | LPYPDGMSPFQAAMVDGSGYQVHRTMQFEDGASLTVNY |
| | | | RYTYEGSHIKGEAQVKGTGFPADGPVMTNSLTAADWCR |
| | | | SKKTYPNDKTIISTFKWSYTTGNGKRYRSTARTTYTFAKP |
| | | | MAANYLKNQPMYVFRKTELKHSKTELNFKEWQKAFTD |
| | | | VMGMDELYKAS* |
| mCherry- | 5 & | 1D | MVSKGEEDNMAIIKEFMRFKVHMEGSVNGHEFEIEGEGE |
| FLAG- | 113 | | GRPYEGTQTAKLKVTKGGPLPFAWDILSPQFMYGSKAYV |
| UAG-UGG- | | | KHPADIPDYLKLSFPEGFKWERVMNFEDGGVVTVTQDSS |
| P2A-T2A- | | | LQDGEFIYKVKLRGTNFPSDGPVMQKKTMGWEASSERM |
| HA- | | | YPEDGALKGEIKQRLKLKDGGHYDAEVKTTYKAKKPVQ |
| mNeonGreen | | | LPGAYNVNIKLDITSHNEDYTIVEQYERAEGRHSTGGMD |
| | | | ELYKDYKDDDDKNSA*RWLPCQRHATSATNFSLLKQAG |
| | | | DVEENPGPGGSEGRGSLLTCGDVEENPGPSGYPYDVPDY |
| | | | AHMVSKGEEDNMASLPATHELHIFGSINGVDFDMVGQG |
| | | | TGNPNDGYEELNLKSTKGDLQFSPWILVPHIGYGFHQYLP |
| | | | YPDGMSPFQAAMVDGSGYQVHRTMQFEDGASLTVNYR |
| | | | YTYEGSHIKGEAQVKGTGFPADGPVMTNSLTAADWCRS |
| | | | KKTYPNDKTIISTFKWSYTTGNGKRYRSTARTTYTFAKP |
| | | | MAANYLKNQPMYVFRKTELKHSKTELNFKEWQKAFTD |
| | | | VMGMDELYKAS* |
| mCherry- | 6 & | 1D | MVSKGEEDNMAIIKEFMRFKVHMEGSVNGHEFEIEGEGE |
| FLAG- | 114 | | GRPYEGTQTAKLKVTKGGPLPFAWDILSPQFMYGSKAYV |
| UAG-UAG- | | | KHPADIPDYLKLSFPEGFKWERVMNFEDGGVVTVTQDSS |
| P2A-T2A- | | | LQDGEFIYKVKLRGTNFPSDGPVMQKKTMGWEASSERM |
| HA- | | | YPEDGALKGEIKQRLKLKDGGHYDAEVKTTYKAKKPVQ |
| mNeonGreen | | | LPGAYNVNIKLDITSHNEDYTIVEQYERAEGRHSTGGMD |
| | | | ELYKDYKDDDDKNSA*R*LPCQRHATSATNFSLLKQAGD |
| | | | VEENPGPGGSEGRGSLLTCGDVEENPGPSGYPYDVPDYA |
| | | | HMVSKGEEDNMASLPATHELHIFGSINGVDFDMVGQGT |
| | | | GNPNDGYEELNLKSTKGDLQFSPWILVPHIGYGFHQYLP |
| | | | YPDGMSPFQAAMVDGSGYQVHRTMQFEDGASLTVNYR |
| | | | YTYEGSHIKGEAQVKGTGFPADGPVMTNSLTAADWCRS |
| | | | KKTYPNDKTIISTFKWSYTTGNGKRYRSTARTTYTFAKP |
| | | | MAANYLKNQPMYVFRKTELKHSKTELNFKEWQKAFTD |
| | | | VMGMDELYKAS* |
| mCherry- | 7 & | 1D | MVSKGEEDNMAIIKEFMRFKVHMEGSVNGHEFEIEGEGE |
| FLAG- | 144 | | GRPYEGTQTAKLKVTKGGPLPFAWDILSPQFMYGSKAYV |
| UAG-UGG- | | | KHPADIPDYLKLSFPEGFKWERVMNFEDGGVVTVTQDSS |
| MS2-P2A- | | | LQDGEFIYKVKLRGTNFPSDGPVMQKKTMGWEASSERM |
| T2A-HA- | | | YPEDGALKGEIKQRLKLKDGGHYDAEVKTTYKAKKPVQ |
| mNeonGreen | | | LPGAYNVNIKLDITSHNEDYTIVEQYERAEGRHSTGGMD |
| | | | ELYKDYKDDDDKNSAWR*LCQRHAKHEDHPCTSATNFS |
| | | | LLKQAGDVEENPGPGGSEGRGSLLTCGDVEENPGPSGYP |
| | | | YDVPDYAHMVSKGEEDNMASLPATHELHIFGSINGVDFD |
| | | | MVGQGTGNPNDGYEELNLKSTKGDLQFSPWILVPHIGYG |
| | | | FHQYLPYPDGMSPFQAAMVDGSGYQVHRTMQFEDGASL |
| | | | TVNYRYTYEGSHIKGEAQVKGTGFPADGPVMTNSLTAA |
| | | | DWCRSKKTYPNDKTIISTFKWSYTTGNGKRYRSTARTTY |
| | | | TFAKPMAANYLKNQPMYVFRKTELKHSKTELNFKEWQK |
| | | | AFTDVMGMDELYKAS* |
| mCherry- | 8 & | 1D | MVSKGEEDNMAIIKEFMRFKVHMEGSVNGHEFEIEGEGE |
| FLAG- | 145 | | GRPYEGTQTAKLKVTKGGPLPFAWDILSPQFMYGSKAYV |
| UAG-UAG- | | | KHPADIPDYLKLSFPEGFKWERVMNFEDGGVVTVTQDSS |
| MS2-P2A- | | | LQDGEFIYKVKLRGTNFPSDGPVMQKKTMGWEASSERM |
| T2A-HA- | | | YPEDGALKGEIKQRLKLKDGGHYDAEVKTTYKAKKPVQ |
| mNeonGreen | | | LPGAYNVNIKLDITSHNEDYTIVEQYERAEGRHSTGGMD |
| | | | ELYKDYKDDDDKNSA*R*LCQRHAKHEDHPCTSATNFSL |
| | | | LKQAGDVEENPGPGGSEGRGSLLTCGDVEENPGPSGYPY |
| | | | DVPDYAHMVSKGEEDNMASLPATHELHIFGSINGVDFD |
| | | | MVGQGTGNPNDGYEELNLKSTKGDLQFSPWILVPHIGYG |
| | | | FHQYLPYPDGMSPFQAAMVDGSGYQVHRTMQFEDGASL |
| | | | TVNYRYTYEGSHIKGEAQVKGTGFPADGPVMTNSLTAA |
| | | | DWCRSKKTYPNDKTIISTFKWSYTTGNGKRYRSTARTTY |
| | | | TFAKPMAANYLKNQPMYVFRKTELKHSKTELNFKEWQK |
| | | | AFTDVMGMDELYKAS* |
| mCherry- | 9 & | 1E | MVSKGEEDNMAIIKEFMRFKVHMEGSVNGHEFEIEGEGE |
| FLAG | 146 | | GRPYEGTQTAKLKVTKGGPLPFAWDILSPQFMYGSKAYV |
| P2A-T2A- | | | KHPADIPDYLKLSFPEGFKWERVMNFEDGGVVTVTQDSS |
| UAG-UAG- | | | LQDGEFIYKVKLRGTNFPSDGPVMQKKTMGWEASSERM |
| PP7-P2A- | | | YPEDGALKGEIKQRLKLKDGGHYDAEVKTTYKAKKPVQ |
| T2A-HA- | | | LPGAYNVNIKLDITSHNEDYTIVEQYERAEGRHSTGGMD |
| mNeonGreen | | | ELYKDYKDDDDKGSGATNFSLLKQAGDVEENPGPASAG |
| | | | SGEGRGSLLTCGDVEENPGPATGNSA*R*LCQRHAKEQTI |
| | | | WRRSNTSATNFSLLKQAGDVEENPGPGGSEGRGSLLTCG |
| | | | DVEENPGPSGYPYDVPDYAHMVSKGEEDNMASLPATHE |
| | | | LHIFGSINGVDFDMVGQGTGNPNDGYEELNLKSTKGDLQ |
| | | | FSPWILVPHIGYGFHQYLPYPDGMSPFQAAMVDGSGYQV |
| | | | HRTMQFEDGASLTVNYRYTYEGSHIKGEAQVKGTGFPA |
| | | | DGPVMTNSLTAADWCRSKKTYPNDKTIISTFKWSYTTGN |
| | | | GKRYRSTARTTYTFAKPMAANYLKNQPMYVFRKTELKH |
| | | | SKTELNFKEWQKAFTDVMGMDELYKAS* |
| mCherry- | 10 | 1E | MVSKGEEDNMAIIKEFMRFKVHMEGSVNGHEFEIEGEGE |
| FLAG | & | | GRPYEGTQTAKLKVTKGGPLPFAWDILSPQFMYGSKAYV |
| P2A-T2A- | 147 | | KHPADIPDYLKLSFPEGFKWERVMNFEDGGVVTVTQDSS |
| UAG-UAG- | | | LQDGEFIYKVKLRGTNFPSDGPVMQKKTMGWEASSERM |
| BoxB-P2A- | | | YPEDGALKGEIKQRLKLKDGGHYDAEVKTTYKAKKPVQ |
| T2A-HA- | | | LPGAYNVNIKLDITSHNEDYTIVEQYERAEGRHSTGGMD |
| mNeonGreen | | | ELYKDYKDDDDKGSGATNFSLLKQAGDVEENPGPASAG |
| | | | SGEGRGSLLTCGDVEENPGPATGNSA*R*LCQRHAVRAL |
| | | | KKGPTSATNFSLLKQAGDVEENPGPGGSEGRGSLLTCGD |
| | | | VEENPGPSGYPYDVPDYAHMVSKGEEDNMASLPATHEL |
| | | | HIFGSINGVDFDMVGQGTGNPNDGYEELNLKSTKGDLQF |
| | | | SPWILVPHIGYGFHQYLPYPDGMSPFQAAMVDGSGYQV |
| | | | HRTMQFEDGASLTVNYRYTYEGSHIKGEAQVKGTGFPA |
| | | | DGPVMTNSLTAADWCRSKKTYPNDKTIISTFKWSYTTGN |
| | | | GKRYRSTARTTYTFAKPMAANYLKNQPMYVFRKTELKH |
| | | | SKTELNFKEWQKAFTDVMGMDELYKAS* |
| mCherry- | 11 | 1E | MVSKGEEDNMAIIKEFMRFKVHMEGSVNGHEFEIEGEGE |
| FLAG | & | | GRPYEGTQTAKLKVTKGGPLPFAWDILSPQFMYGSKAYV |
| P2A-T2A- | 148 | | KHPADIPDYLKLSFPEGFKWERVMNFEDGGVVTVTQDSS |
| UAG-UAG- | | | LQDGEFIYKVKLRGTNFPSDGPVMQKKTMGWEASSERM |
| HIV TAR- | | | YPEDGALKGEIKQRLKLKDGGHYDAEVKTTYKAKKPVQ |
| P2A-T2A- | | | LPGAYNVNIKLDITSHNEDYTIVEQYERAEGRHSTGGMD |
| HA- | | | ELYKDYKDDDDKGSGATNFSLLKQAGDVEENPGPASAG |
| mNeonGreen | | | SGEGRGSLLTCGDVEENPGPATGNSA*R*LCQRHAVGSSE |
| | | | LISSEPTSATNFSLLKQAGDVEENPGPGGSEGRGSLLTCG |
| | | | DVEENPGPSGYPYDVPDYAHMVSKGEEDNMASLPATHE |
| | | | LHIFGSINGVDFDMVGQGTGNPNDGYEELNLKSTKGDLQ |
| | | | FSPWILVPHIGYGFHQYLPYPDGMSPFQAAMVDGSGYQV |
| | | | HRTMQFEDGASLTVNYRYTYEGSHIKGEAQVKGTGFPA |
| | | | DGPVMTNSLTAADWCRSKKTYPNDKTIISTFKWSYTTGN |
| | | | GKRYRSTARTTYTFAKPMAANYLKNQPMYVFRKTELKH |
| | | | SKTELNFKEWQKAFTDVMGMDELYKAS* |
| PCP-linker- | 12 | 1E | MSKTIVLSVGEATRTLTEIQSTADRQIFEEKVGPLVGRLRL |
| ADAR2 | | | TASLRQNGAKTAYRVNLKLDQADVVDSGLPKVRYTQV |
| (E488Q) DD- | | | WSHDVTIVANSTEASRKSLYDLTKSLVATSQVEDLVVNL |
| TagBFP | | | VPLGRASTGSGIYGGSGSGAGSGSPAGGGAPGSGGGSQL |
| | | | HLPQVLADAVSRLVLGKFGDLTDNFSSPHARRKVLAGV |
| | | | VMTTGTDVKDAKVISVSTGTKCINGEYMSDRGLALNDC |
| | | | HAEIISRRSLLRFLYTQLELYLNNKDDQKRSIFQKSERGGF |
| | | | RLKENVQFHLYISTSPCGDARIFSPHEPILEEPADRHPNRK |
| | | | ARGQLRTKIESGQGTIPVRSNASIQTWDGVLQGERLLTMS |
| | | | CSDKIARWNVVGIQGSLLSIFVEPIYFSSIILGSLYHGDHLS |
| | | | RAMYQRISNIEDLPPLYTLNKPLLSGISNAEARQPGKAPN |
| | | | FSVNWTVGDSAIEVINATTGKDELGRASRLCKHALYCRW |
| | | | MRVHGKVPSHLLRSKITKPNVYHESKLAAKEYQAAKAR |
| | | | LFTAFIKAGLGAWVEKPTEQDQFSLTGSGSSELIKENMH |
| | | | MKLYMEGTVDNHHFKCTSEGEVTTYEDGGVLTATQDTS |
| | | | LQDGCLIYNVKIRGVNFTSNGPVMQKKTLGWEAFTETLY |
| | | | PADGGLEGRNDMALKLVGGSHLIANIKTTYRSKKPAKNL |
| | | | KMPGVYYVDYRLERIKEANNETYVEQHEVAVARYCDLP |
| | | | SKLGHKLN* |
| λN-linker- | 13 | 1E | MADAQTRRRERRAEKQAQWKAANTGSGIYGGSGSGAG |
| ADAR2 | | | SGSPAGGGAPGSGGGSQLHLPQVLADAVSRLVLGKFGDL |
| (E488Q) DD- | | | TDNFSSPHARRKVLAGVVMTTGTDVKDAKVISVSTGTKC |
| TagBFP | | | INGEYMSDRGLALNDCHAEIISRRSLLRFLYTQLELYLNN |
| | | | KDDQKRSIFQKSERGGFRLKENVQFHLYISTSPCGDARIFS |
| | | | PHEPILEEPADRHPNRKARGQLRTKIESGQGTIPVRSNASI |
| | | | QTWDGVLQGERLLTMSCSDKIARWNVVGIQGSLLSIFVE |
| | | | PIYFSSIILGSLYHGDHLSRAMYQRISNIEDLPPLYTLNKPL |
| | | | LSGISNAEARQPGKAPNFSVNWTVGDSAIEVINATTGKDE |
| | | | LGRASRLCKHALYCRWMRVHGKVPSHLLRSKITKPNVY |
| | | | HESKLAAKEYQAAKARLFTAFIKAGLGAWVEKPTEQDQ |
| | | | FSLTGSGSSELIKENMHMKLYMEGTVDNHHFKCTSEGEV |
| | | | TTYEDGGVLTATQDTSLQDGCLIYNVKIRGVNFTSNGPV |
| | | | MQKKTLGWEAFTETLYPADGGLEGRNDMALKLVGGSH |
| | | | LIANIKTTYRSKKPAKNLKMPGVYYVDYRLERIKEANNE |
| | | | TYVEQHEVAVARYCDLPSKLGHKLN* |
| HIV-tat- | 14 | 1E | MASGPRPRGTRGKGRRIRRTGSGIYGGSGSGAGSGSPAG |
| linker- | | | GGAPGSGGGSQLHLPQVLADAVSRLVLGKFGDLTDNFSS |
| ADAR2 | | | PHARRKVLAGVVMTTGTDVKDAKVISVSTGTKCINGEY |
| (E488Q) DD- | | | MSDRGLALNDCHAEIISRRSLLRFLYTQLELYLNNKDDQ |
| TagBFP | | | KRSIFQKSERGGFRLKENVQFHLYISTSPCGDARIFSPHEPI |
| | | | LEEPADRHPNRKARGQLRTKIESGQGTIPVRSNASIQTWD |
| | | | GVLQGERLLTMSCSDKIARWNVVGIQGSLLSIFVEPIYFSS |
| | | | IILGSLYHGDHLSRAMYQRISNIEDLPPLYTLNKPLLSGISN |
| | | | AEARQPGKAPNFSVNWTVGDSAIEVINATTGKDELGRAS |
| | | | RLCKHALYCRWMRVHGKVPSHLLRSKITKPNVYHESKL |
| | | | AAKEYQAAKARLFTAFIKAGLGAWVEKPTEQDQFSLTGS |
| | | | GSSELIKENMHMKLYMEGTVDNHHFKCTSEGEVTTYED |
| | | | GGVLTATQDTSLQDGCLIYNVKIRGVNFTSNGPVMQKKT |
| | | | LGWEAFTETLYPADGGLEGRNDMALKLVGGSHLIANIKT |
| | | | TYRSKKPAKNLKMPGVYYVDYRLERIKEANNETYVEQH |
| | | | EVAVARYCDLPSKLGHKLN* |
| dTomato | 15 | 2B, 2D, 3H | MVSKGEEVIKEFMRFKVRMEGSMNGHEFEIEGEGEGRPY |
| | | | EGTQTAKLKVTKGGPLPFAWDILSPQFMYGSKAYVKHP |
| | | | ADIPDYKKLSFPEGFKWERVMNFEDGGLVTVTQDSSLQD |
| | | | GTLIYKVKMRGTNFPPDGPVMQKKTMGWEASTERLYPR |
| | | | DGVLKGEIHQALKLKDGGHYLVEFKTIYMAKKPVQLPG |
| | | | YYYVDTKLDITSHNEDYTIVEQYERSEGRHHLFLYGMDE |
| | | | LYK |
| EGFPd2- | 16 | 2B, 2D, 3H | MVSKGEELFTGVVPILVELDGDVNGHKFSVSGEGEGDAT |
| UAG-UAG- | & | | YGKLTLKFICTTGKLPVPWPTLVTTLTYGVQCFSRYPDH |
| MS2-polyA | 149 | | MKQHDFFKSAMPEGYVQERTIFFKDDGNYKTRAEVKFE |
| | | | GDTLVNRIELKGIDFKEDGNILGHKLEYNYNSHNVYIMA |
| | | | DKQKNGIKVNFKIRHNIEDGSVQLADHYQQNTPIGDGPV |
| | | | LLPDNHYLSTQSALSKDPNEKRDHMVLLEFVTAAGITLG |
| | | | MDELYKKLSHGFPPEVEEQDDGTLPMSCAQESGMDRHP |
| | | | AACASARINV*R*LCQRHTKHEDHPCRPHSSGAGCLSEG |
| | | | GGWCGQCPGSQIPLRSFSLCQKLWGHHEAPWASDFWLIK |
| | | | EIYF . . . |
| MCP-linker- | 17 | 3D | MASNFTQFVLVDNGGTGDVTVAPSNFANGIAEWISSNSR |
| ADAR2- | | | SQAYKVTCSVRQSSAQNRKYTIKVEVPKGAWRSYLNME |
| DDN- | | | LTIPIFATNSDCELIVKAMQGLLKDGNPIPSAIAANSGIYG |
| Bad(F)- | | | GSGSGAGSGSPAGGGAPGSGGGSQLHLPQVLADAVSRLV |
| ADAR2 | | | LGKFGDLTDNFSSPHARRKVLAGVVMTTGTDVKDAKVI |
| (E488Q)-DDC- | | | SVSTGTKCINGEYMSDRGLALNDCHAEIISRRSLLRFLYT |
| TagBFP | | | QLELYLNNKDDQKRSIFQKSERGGFRLKENVQFHLYISTS |
| | | | PCGDARIFSPHEPILEEPAASGSGTGAPPNLWAAQRYGRE |
| | | | LRRMSDEFVDRHPNRKARGQLRTKIESGQGTIPVRSNASI |
| | | | QTWDGVLQGERLLTMSCSDKIARWNVVGIQGSLLSIFVE |
| | | | PIYFSSIILGSLYHGDHLSRAMYQRISNIEDLPPLYTLNKPL |
| | | | LSGISNAEARQPGKAPNFSVNWTVGDSAIEVINATTGKDE |
| | | | LGRASRLCKHALYCRWMRVHGKVPSHLLRSKITKPNVY |
| | | | HESKLAAKEYQAAKARLFTAFIKAGLGAWVEKPTEQDQ |
| | | | FSLTGSGSSELIKENMHMKLYMEGTVDNHHFKCTSEGEG |
| | | | KPYEGTQTMRIKVVEGGPLPFAFDILATSFLYGSKTFINHT |
| | | | QGIPDFFKQSFPEGFTWERVTTYEDGGVLTATQDTSLQD |
| | | | GCLIYNVKIRGVNFTSNGPVMQKKTLGWEAFTETLYPAD |
| | | | GGLEGRNDMALKLVGGSHLIANIKTTYRSKKPAKNLKM |
| | | | PGVYYVDYRLERIKEANNETYVEQHEVAVARYCDLPSK |
| | | | LGHKLN* |
| | | | MASNFTQFVLVDNGGTGDVTVAPSNFANGIAEWISSNSR |
| MCP-linker- | 18 | 3D-F | SQAYKVTCSVRQSSAQNRKYTIKVEVPKGAWRSYLNME |
| ADAR2- | | | LTIPIFATNSDCELIVKAMQGLLKDGNPIPSAIAANSGIYG |
| DDN- | | | GSGSGAGSGSPAGGGAPGSGGGSQLHLPQVLADAVSRLV |
| Bad(F)- | | | LGKFGDLTDNFSSPHARRKVLAGVVMTTGTDVKDAKVI |
| ADAR2 | | | SVSTGTKCINGEYMSDRGLALNDCHAEIISRRSLLRFLYT |
| 88Q)-DDC- | | | QLELYLNNKDDQKRSIFQKSERGGFRLKENVQFHLYISTS |
| (E4Bcl-xL- | | | PCGDARIFSPHEPILEEPAASGSGTGAPPNLWAAQRYGRE |
| TagBFP | | | LRRMSDEFVDRHPNRKARGQLRTKIESGQGTIPVRSNASI |
| | | | QTWDGVLQGERLLTMSCSDKIARWNVVGIQGSLLSIFVE |
| | | | PIYFSSIILGSLYHGDHLSRAMYQRISNIEDLPPLYTLNKPL |
| | | | LSGISNAEARQPGKAPNFSVNWTVGDSAIEVINATTGKDE |
| | | | LGRASRLCKHALYCRWMRVHGKVPSHLLRSKITKPNVY |
| | | | HESKLAAKEYQAAKARLFTAFIKAGLGAWVEKPTEQDQ |
| | | | FSLTGSAAGGSGGSAAASSNRELVVDFLSYKLSQKGYSW |
| | | | SQFSDVEENRTEAPEGTESEMETPSAINGNPSWHLADSPA |
| | | | VNGATGHSSSLDAREVIPMAAVKQALREAGDEFELRYRR |
| | | | AFSDLTSQLHITPGTAYQSFEQVVNELFRDGVNWGRIVAF |
| | | | FSFGGALCVESVDKEMQVLVSRIAAWMATYLNDHLEPW |
| | | | IQENGGWDTFVELYGNNGSSELIKENMHMKLYMEGTVD |
| | | | NHHFKCTSEGEGKPYEGTQTMRIKVVEGGPLPFAFDILAT |
| | | | SFLYGSKTFINHTQGIPDFFKQSFPEGFTWERVTTYEDGG |
| | | | VLTATQDTSLQDGCLIYNVKIRGVNFTSNGPVMQKKTLG |
| | | | WEAFTETLYPADGGLEGRNDMALKLVGGSHLIANIKTTY |
| | | | RSKKPAKNLKMPGVYYVDYRLERIKEANNETYVEQHEV |
| | | | AVARYCDLPSKLGHKLN* |
| MCP-linker- | 19 | 3D | MASNFTQFVLVDNGGTGDVTVAPSNFANGIAEWISSNSR |
| ADAR2- | | | SQAYKVTCSVRQSSAQNRKYTIKVEVPKGAWRSYLNME |
| DDN-Bim- | | | LTIPIFATNSDCELIVKAMQGLLKDGNPIPSAIAANSGIYG |
| ADAR2 | | | GSGSGAGSGSPAGGGAPGSGGGSQLHLPQVLADAVSRLV |
| (E488Q)-DDC- | | | LGKFGDLTDNFSSPHARRKVLAGVVMTTGTDVKDAKVI |
| TagBFP | | | SVSTGTKCINGEYMSDRGLALNDCHAEIISRRSLLRFLYT |
| | | | QLELYLNNKDDQKRSIFQKSERGGFRLKENVQFHLYISTS |
| | | | PCGDARIFSPHEPILEEPAASGSGSGDMRPEIWIAQELRRI |
| | | | GDEFNAYYARRTGDRHPNRKARGQLRTKIESGQGTIPVR |
| | | | SNASIQTWDGVLQGERLLTMSCSDKIARWNVVGIQGSLL |
| | | | SIFVEPIYFSSIILGSLYHGDHLSRAMYQRISNIEDLPPLYTL |
| | | | NKPLLSGISNAEARQPGKAPNFSVNWTVGDSAIEVINATT |
| | | | GKDELGRASRLCKHALYCRWMRVHGKVPSHLLRSKITK |
| | | | PNVYHESKLAAKEYQAAKARLFTAFIKAGLGAWVEKPT |
| | | | EQDQFSLTGSGSSELIKENMHMKLYMEGTVDNHHFKCTS |
| | | | EGEGKPYEGTQTMRIKVVEGGPLPFAFDILATSFLYGSKT |
| | | | FINHTQGIPDFFKQSFPEGFTWERVTTYEDGGVLTATQDT |
| | | | SLQDGCLIYNVKIRGVNFTSNGPVMQKKTLGWEAFTETL |
| | | | YPADGGLEGRNDMALKLVGGSHLIANIKTTYRSKKPAKN |
| | | | LKMPGVYYVDYRLERIKEANNETYVEQHEVAVARYCDL |
| | | | PSKLGHKLN* |
| MCP-linker- | 20 | 3D | MASNFTQFVLVDNGGTGDVTVAPSNFANGIAEWISSNSR |
| ADAR2- | | | SQAYKVTCSVRQSSAQNRKYTIKVEVPKGAWRSYLNME |
| DDN-Bim- | | | LTIPIFATNSDCELIVKAMQGLLKDGNPIPSAIAANSGIYG |
| ADAR2 | | | GSGSGAGSGSPAGGGAPGSGGGSQLHLPQVLADAVSRLV |
| (E488Q)-DDC- | | | LGKFGDLTDNFSSPHARRKVLAGVVMTTGTDVKDAKVI |
| Bcl-xL- | | | SVSTGTKCINGEYMSDRGLALNDCHAEIISRRSLLRFLYT |
| TagBFP | | | QLELYLNNKDDQKRSIFQKSERGGFRLKENVQFHLYISTS |
| | | | PCGDARIFSPHEPILEEPAASGSGSGDMRPEIWIAQELRRI |
| | | | GDEFNAYYARRTGDRHPNRKARGQLRTKIESGQGTIPVR |
| | | | SNASIQTWDGVLQGERLLTMSCSDKIARWNVVGIQGSLL |
| | | | SIFVEPIYFSSIILGSLYHGDHLSRAMYQRISNIEDLPPLYTL |
| | | | NKPLLSGISNAEARQPGKAPNFSVNWTVGDSAIEVINATT |
| | | | GKDELGRASRLCKHALYCRWMRVHGKVPSHLLRSKITK |
| | | | PNVYHESKLAAKEYQAAKARLFTAFIKAGLGAWVEKPT |
| | | | EQDQFSLTGSAAGGSGGSAAASSNRELVVDFLSYKLSQK |
| | | | GYSWSQFSDVEENRTEAPEGTESEMETPSAINGNPSWHL |
| | | | ADSPAVNGATGHSSSLDAREVIPMAAVKQALREAGDEFE |
| | | | LRYRRAFSDLTSQLHITPGTAYQSFEQVVNELFRDGVNW |
| | | | GRIVAFFSFGGALCVESVDKEMQVLVSRIAAWMATYLN |
| | | | DHLEPWIQENGGWDTFVELYGNNGSSELIKENMHMKLY |
| | | | MEGTVDNHHFKCTSEGEGKPYEGTQTMRIKVVEGGPLPF |
| | | | AFDILATSFLYGSKTFINHTQGIPDFFKQSFPEGFTWERVT |
| | | | TYEDGGVLTATQDTSLQDGCLIYNVKIRGVNFTSNGPVM |
| | | | QKKTLGWEAFTETLYPADGGLEGRNDMALKLVGGSHLI |
| | | | ANIKTTYRSKKPAKNLKMPGVYYVDYRLERIKEANNETY |
| | | | VEQHEVAVARYCDLPSKLGHKLN* |
| MCP-linker- | 21 | 3D,3H | MASNFTQFVLVDNGGTGDVTVAPSNFANGIAEWISSNSR |
| ADAR2- | | | SQAYKVTCSVRQSSAQNRKYTIKVEVPKGAWRSYLNME |
| DDN- | | | LTIPIFATNSDCELIVKAMQGLLKDGNPIPSAIAANSGIYG |
| MS1(A)- | | | GSGSGAGSGSPAGGGAPGSGGGSQLHLPQVLADAVSRLV |
| ADAR2 | | | LGKFGDLTDNFSSPHARRKVLAGVVMTTGTDVKDAKVI |
| (E488Q)-DDC- | | | SVSTGTKCINGEYMSDRGLALNDCHAEIISRRSLLRFLYT |
| TagBFP | | | QLELYLNNKDDQKRSIFQKSERGGFRLKENVQFHLYISTS |
| | | | PCGDARIFSPHEPILEEPAASGGSGGSGRPEIWMTQGLRRL |
| | | | GDEANAYYARRTGDRHPNRKARGQLRTKIESGQGTIPVR |
| | | | SNASIQTWDGVLQGERLLTMSCSDKIARWNVVGIQGSLL |
| | | | SIFVEPIYFSSIILGSLYHGDHLSRAMYQRISNIEDLPPLYTL |
| | | | NKPLLSGISNAEARQPGKAPNFSVNWTVGDSAIEVINATT |
| | | | GKDELGRASRLCKHALYCRWMRVHGKVPSHLLRSKITK |
| | | | PNVYHESKLAAKEYQAAKARLFTAFIKAGLGAWVEKPT |
| | | | EQDQFSLTGSGSSELIKENMHMKLYMEGTVDNHHFKCTS |
| | | | EGEGKPYEGTQTMRIKVVEGGPLPFAFDILATSFLYGSKT |
| | | | FINHTQGIPDFFKQSFPEGFTWERVTTYEDGGVLTATQDT |
| | | | SLQDGCLIYNVKIRGVNFTSNGPVMQKKTLGWEAFTETL |
| | | | YPADGGLEGRNDMALKLVGGSHLIANIKTTYRSKKPAKN |
| | | | LKMPGVYYVDYRLERIKEANNETYVEQHEVAVARYCDL |
| | | | PSKLGHKLN* |
| MCP-linker- | 22 | 3D-E, | MASNFTQFVLVDNGGTGDVTVAPSNFANGIAEWISSNSR |
| ADAR2- | | 3G-H | SQAYKVTCSVRQSSAQNRKYTIKVEVPKGAWRSYLNME |
| DDN- | | | LTIPIFATNSDCELIVKAMQGLLKDGNPIPSAIAANSGIYG |
| MS1(A)- | | | GSGSGAGSGSPAGGGAPGSGGGSQLHLPQVLADAVSRLV |
| ADAR2 | | | LGKFGDLTDNFSSPHARRKVLAGVVMTTGTDVKDAKVI |
| (E488Q)-DDC- | | | SVSTGTKCINGEYMSDRGLALNDCHAEIISRRSLLRFLYT |
| Mcl-1- | | | QLELYLNNKDDQKRSIFQKSERGGFRLKENVQFHLYISTS |
| TagBFP | | | PCGDARIFSPHEPILEEPAASGGSGGSGRPEIWMTQGLRRL |
| | | | GDEANAYYARRTGDRHPNRKARGQLRTKIESGQGTIPVR |
| | | | SNASIQTWDGVLQGERLLTMSCSDKIARWNVVGIQGSLL |
| | | | SIFVEPIYFSSIILGSLYHGDHLSRAMYQRISNIEDLPPLYTL |
| | | | NKPLLSGISNAEARQPGKAPNFSVNWTVGDSAIEVINATT |
| | | | GKDELGRASRLCKHALYCRWMRVHGKVPSHLLRSKITK |
| | | | PNVYHESKLAAKEYQAAKARLFTAFIKAGLGAWVEKPT |
| | | | EQDQFSLTGSGTGGPGDELYRQSLEIISRYLREQATGAKD |
| | | | TKPMGRSGATSRKALETLRRVGDGVQRNHETAFQGMLR |
| | | | KLDIKNEDDVKSLSRVMIHVFSDGVTNWGRIVTLISFGAF |
| | | | VAKHLKTINQESCIEPLAESITDVLVRTKRDWLVKQRGW |
| | | | DGFVEFFHVEDLEGGGSSELIKENMHMKLYMEGTVDNH |
| | | | HFKCTSEGEGKPYEGTQTMRIKVVEGGPLPFAFDILATSF |
| | | | LYGSKTFINHTQGIPDFFKQSFPEGFTWERVTTYEDGGVL |
| | | | TATQDTSLQDGCLIYNVKIRGVNFTSNGPVMQKKTLGWE |
| | | | AFTETLYPADGGLEGRNDMALKLVGGSHLIANIKTTYRS |
| | | | KKPAKNLKMPGVYYVDYRLERIKEANNETYVEQHEVAV |
| | | | ARYCDLPSKLGHKLN* |
| MCP-linker- | 23 | 3F | MASNFTQFVLVDNGGTGDVTVAPSNFANGIAEWISSNSR |
| ADAR2- | | | SQAYKVTCSVRQSSAQNRKYTIKVEVPKGAWRSYLNME |
| DDN- | | | LTIPIFATNSDCELIVKAMQGLLKDGNPIPSAIAANSGIYG |
| Bad(L)- | | | GSGSGAGSGSPAGGGAPGSGGGSQLHLPQVLADAVSRLV |
| ADAR2 | | | LGKFGDLTDNFSSPHARRKVLAGVVMTTGTDVKDAKVI |
| (E488Q)-DDC- | | | SVSTGTKCINGEYMSDRGLALNDCHAEIISRRSLLRFLYT |
| Bcl-xL- | | | QLELYLNNKDDQKRSIFQKSERGGFRLKENVQFHLYISTS |
| TagBFP | | | PCGDARIFSPHEPILEEPAASGSGTGAPPNLWAAQRYGRE |
| | | | LRRMSDELVDRHPNRKARGQLRTKIESGQGTIPVRSNASI |
| | | | QTWDGVLQGERLLTMSCSDKIARWNVVGIQGSLLSIFVE |
| | | | PIYFSSIILGSLYHGDHLSRAMYQRISNIEDLPPLYTLNKPL |
| | | | LSGISNAEARQPGKAPNFSVNWTVGDSAIEVINATTGKDE |
| | | | LGRASRLCKHALYCRWMRVHGKVPSHLLRSKITKPNVY |
| | | | HESKLAAKEYQAAKARLFTAFIKAGLGAWVEKPTEQDQ |
| | | | FSLTGSAAGGSGGSAAASSNRELVVDFLSYKLSQKGYSW |
| | | | SQFSDVEENRTEAPEGTESEMETPSAINGNPSWHLADSPA |
| | | | VNGATGHSSSLDAREVIPMAAVKQALREAGDEFELRYRR |
| | | | AFSDLTSQLHITPGTAYQSFEQVVNELFRDGVNWGRIVAF |
| | | | FSFGGALCVESVDKEMQVLVSRIAAWMATYLNDHLEPW |
| | | | IQENGGWDTFVELYGNNGSSELIKENMHMKLYMEGTVD |
| | | | NHHFKCTSEGEGKPYEGTQTMRIKVVEGGPLPFAFDILAT |
| | | | SFLYGSKTFINHTQGIPDFFKQSFPEGFTWERVTTYEDGG |
| | | | VLTATQDTSLQDGCLIYNVKIRGVNFTSNGPVMQKKTLG |
| | | | WEAFTETLYPADGGLEGRNDMALKLVGGSHLIANIKTTY |
| | | | RSKKPAKNLKMPGVYYVDYRLERIKEANNETYVEQHEV |
| | | | AVARYCDLPSKLGHKLN* |
| MCP-linker- | 24 | 3G | MASNFTQFVLVDNGGTGDVTVAPSNFANGIAEWISSNSR |
| ADAR2- | | | SQAYKVTCSVRQSSAQNRKYTIKVEVPKGAWRSYLNME |
| DDN- | | | LTIPIFATNSDCELIVKAMQGLLKDGNPIPSAIAANSGIYG |
| MS1(I)- | | | GSGSGAGSGSPAGGGAPGSGGGSQLHLPQVLADAVSRLV |
| ADAR2 | | | LGKFGDLTDNFSSPHARRKVLAGVVMTTGTDVKDAKVI |
| (E488Q)-DDC- | | | SVSTGTKCINGEYMSDRGLALNDCHAEIISRRSLLRFLYT |
| Mcl-1- | | | QLELYLNNKDDQKRSIFQKSERGGFRLKENVQFHLYISTS |
| TagBFP | | | PCGDARIFSPHEPILEEPAASGGSGGSGRPEIWMTQGLRRL |
| | | | GDEINAYYARRTGDRHPNRKARGQLRTKIESGQGTIPVR |
| | | | SNASIQTWDGVLQGERLLTMSCSDKIARWNVVGIQGSLL |
| | | | SIFVEPIYFSSIILGSLYHGDHLSRAMYQRISNIEDLPPLYTL |
| | | | NKPLLSGISNAEARQPGKAPNFSVNWTVGDSAIEVINATT |
| | | | GKDELGRASRLCKHALYCRWMRVHGKVPSHLLRSKITK |
| | | | PNVYHESKLAAKEYQAAKARLFTAFIKAGLGAWVEKPT |
| | | | EQDQFSLTGSGTGGPGDELYRQSLEIISRYLREQATGAKD |
| | | | TKPMGRSGATSRKALETLRRVGDGVQRNHETAFQGMLR |
| | | | KLDIKNEDDVKSLSRVMIHVFSDGVTNWGRIVTLISFGAF |
| | | | VAKHLKTINQESCIEPLAESITDVLVRTKRDWLVKQRGW |
| | | | DGFVEFFHVEDLEGGGSSELIKENMHMKLYMEGTVDNH |
| | | | HFKCTSEGEGKPYEGTQTMRIKVVEGGPLPFAFDILATSF |
| | | | LYGSKTFINHTQGIPDFFKQSFPEGFTWERVTTYEDGGVL |
| | | | TATQDTSLQDGCLIYNVKIRGVNFTSNGPVMQKKTLGWE |
| | | | AFTETLYPADGGLEGRNDMALKLVGGSHLIANIKTTYRS |
| | | | KKPAKNLKMPGVYYVDYRLERIKEANNETYVEQHEVAV |
| | | | ARYCDLPSKLGHKLN* |
| MCP-linker- | 25 | 4C, 4E | MASNFTQFVLVDNGGTGDVTVAPSNFANGIAEWISSNSR |
| ADAR2- | | | SQAYKVTCSVRQSSAQNRKYTIKVEVPKGAWRSYLNME |
| DDN- | | | LTIPIFATNSDCELIVKAMQGLLKDGNPIPSAIAANSGIYG |
| ALFA- | | | GSGSGAGSGSPAGGGAPGSGGGSQLHLPQVLADAVSRLV |
| ADAR2 | | | LGKFGDLTDNFSSPHARRKVLAGVVMTTGTDVKDAKVI |
| (E488Q)-DDC- | | | SVSTGTKCINGEYMSDRGLALNDCHAEIISRRSLLRFLYT |
| TagBFP | | | QLELYLNNKDDQKRSIFQKSERGGFRLKENVQFHLYISTS |
| | | | PCGDARIFSPHEPILEEPAASPSRLEEELRRRLTEPTGDRH |
| | | | PNRKARGQLRTKIESGQGTIPVRSNASIQTWDGVLQGERL |
| | | | LTMSCSDKIARWNVVGIQGSLLSIFVEPIYFSSIILGSLYHG |
| | | | DHLSRAMYQRISNIEDLPPLYTLNKPLLSGISNAEARQPG |
| | | | KAPNFSVNWTVGDSAIEVINATTGKDELGRASRLCKHAL |
| | | | YCRWMRVHGKVPSHLLRSKITKPNVYHESKLAAKEYQA |
| | | | AKARLFTAFIKAGLGAWVEKPTEQDQFSLTGSGSSELIKE |
| | | | NMHMKLYMEGTVDNHHFKCTSEGEGKPYEGTQTMRIK |
| | | | VVEGGPLPFAFDILATSFLYGSKTFINHTQGIPDFFKQSFPE |
| | | | GFTWERVTTYEDGGVLTATQDTSLQDGCLIYNVKIRGVN |
| | | | FTSNGPVMQKKTLGWEAFTETLYPADGGLEGRNDMALK |
| | | | LVGGSHLIANIKTTYRSKKPAKNLKMPGVYYVDYRLERI |
| | | | KEANNETYVEQHEVAVARYCDLPSKLGHKLN* |
| MCP-linker- | 26 | 4C-E | MASNFTQFVLVDNGGTGDVTVAPSNFANGIAEWISSNSR |
| ADAR2- | | | SQAYKVTCSVRQSSAQNRKYTIKVEVPKGAWRSYLNME |
| DDN- | | | LTIPIFATNSDCELIVKAMQGLLKDGNPIPSAIAANSGIYG |
| ALFA- | | | GSGSGAGSGSPAGGGAPGSGGGSQLHLPQVLADAVSRLV |
| ADAR2 | | | LGKFGDLTDNFSSPHARRKVLAGVVMTTGTDVKDAKVI |
| (E488Q)-DDC- | | | SVSTGTKCINGEYMSDRGLALNDCHAEIISRRSLLRFLYT |
| NbALFA- | | | QLELYLNNKDDQKRSIFQKSERGGFRLKENVQFHLYISTS |
| TagBFP | | | PCGDARIFSPHEPILEEPAASPSRLEEELRRRLTEPTGDRH |
| | | | PNRKARGQLRTKIESGQGTIPVRSNASIQTWDGVLQGERL |
| | | | LTMSCSDKIARWNVVGIQGSLLSIFVEPIYFSSIILGSLYHG |
| | | | DHLSRAMYQRISNIEDLPPLYTLNKPLLSGISNAEARQPG |
| | | | KAPNFSVNWTVGDSAIEVINATTGKDELGRASRLCKHAL |
| | | | YCRWMRVHGKVPSHLLRSKITKPNVYHESKLAAKEYQA |
| | | | AKARLFTAFIKAGLGAWVEKPTEQDQFSLTGSGGTAEVQ |
| | | | LQESGGGLVQPGGSLRLSCTASGVTISALNAMAMGWYR |
| | | | QAPGERRVMVAAVSERGNAMYRESVQGRFTVTRDFTNK |
| | | | MVSLQMDNLKPEDTAVYYCHVLEDRVDSFHDYWGQGT |
| | | | QVTVSSGAGSSELIKENMHMKLYMEGTVDNHHFKCTSE |
| | | | GEGKPYEGTQTMRIKVVEGGPLPFAFDILATSFLYGSKTFI |
| | | | NHTQGIPDFFKQSFPEGFTWERVTTYEDGGVLTATQDTSL |
| | | | QDGCLIYNVKIRGVNFTSNGPVMQKKTLGWEAFTETLYP |
| | | | ADGGLEGRNDMALKLVGGSHLIANIKTTYRSKKPAKNL |
| | | | KMPGVYYVDYRLERIKEANNETYVEQHEVAVARYCDLP |
| | | | SKLGHKLN* |
| MCP-linker- | 27 | 4C, 4E | MASNFTQFVLVDNGGTGDVTVAPSNFANGIAEWISSNSR |
| ADAR2- | | | SQAYKVTCSVRQSSAQNRKYTIKVEVPKGAWRSYLNME |
| DDN- | | | LTIPIFATNSDCELIVKAMQGLLKDGNPIPSAIAANSGIYG |
| ALFA-PE- | | | GSGSGAGSGSPAGGGAPGSGGGSQLHLPQVLADAVSRLV |
| ADAR2 | | | LGKFGDLTDNFSSPHARRKVLAGVVMTTGTDVKDAKVI |
| (E488Q)-DDC- | | | SVSTGTKCINGEYMSDRGLALNDCHAEIISRRSLLRFLYT |
| TagBFP | | | QLELYLNNKDDQKRSIFQKSERGGFRLKENVQFHLYISTS |
| | | | PCGDARIFSPHEPILEEPAASGSGPGRLEEELRRRLSPGT |
| | | | GDRHPNRKARGQLRTKIESGQGTIPVRSNASIQTWDGVL |
| | | | QGERLLTMSCSDKIARWNVVGIQGSLLSIFVEPIYFSSIILG |
| | | | SLYHGDHLSRAMYQRISNIEDLPPLYTLNKPLLSGISNAE |
| | | | ARQPGKAPNFSVNWTVGDSAIEVINATTGKDELGRASRL |
| | | | CKHALYCRWMRVHGKVPSHLLRSKITKPNVYHESKLAA |
| | | | KEYQAAKARLFTAFIKAGLGAWVEKPTEQDQFSLTGSGS |
| | | | SELIKENMHMKLYMEGTVDNHHFKCTSEGEGKPYEGTQ |
| | | | TMRIKVVEGGPLPFAFDILATSFLYGSKTFINHTQGIPDFF |
| | | | KQSFPEGFTWERVTTYEDGGVLTATQDTSLQDGCLIYNV |
| | | | KIRGVNFTSNGPVMQKKTLGWEAFTETLYPADGGLEGR |
| | | | NDMALKLVGGSHLIANIKTTYRSKKPAKNLKMPGVYYV |
| | | | DYRLERIKEANNETYVEQHEVAVARYCDLPSKLGHKLN* |
| MCP-linker- | 28 | 4C, 4E | MASNFTQFVLVDNGGTGDVTVAPSNFANGIAEWISSNSR |
| ADAR2- | | | SQAYKVTCSVRQSSAQNRKYTIKVEVPKGAWRSYLNME |
| DDN- | | | LTIPIFATNSDCELIVKAMQGLLKDGNPIPSAIAANSGIYG |
| ALFA-PE- | | | GSGSGAGSGSPAGGGAPGSGGGSQLHLPQVLADAVSRLV |
| ADAR2 | | | LGKFGDLTDNFSSPHARRKVLAGVVMTTGTDVKDAKVI |
| (E488Q)-DDC- | | | SVSTGTKCINGEYMSDRGLALNDCHAEIISRRSLLRFLYT |
| NbALFA- | | | QLELYLNNKDDQKRSIFQKSERGGFRLKENVQFHLYISTS |
| TagBFP | | | PCGDARIFSPHEPILEEPAASGSGPGRLEEELRRRLSPGT |
| | | | GDRHPNRKARGQLRTKIESGQGTIPVRSNASIQTWDGVL |
| | | | QGERLLTMSCSDKIARWNVVGIQGSLLSIFVEPIYFSSIILG |
| | | | SLYHGDHLSRAMYQRISNIEDLPPLYTLNKPLLSGISNAE |
| | | | ARQPGKAPNFSVNWTVGDSAIEVINATTGKDELGRASRL |
| | | | CKHALYCRWMRVHGKVPSHLLRSKITKPNVYHESKLAA |
| | | | KEYQAAKARLFTAFIKAGLGAWVEKPTEQDQFSLTGSGG |
| | | | TAEVQLQESGGGLVQPGGSLRLSCTASGVTISALNAMAM |
| | | | GWYRQAPGERRVMVAAVSERGNAMYRESVQGRFTVTR |
| | | | DFTNKMVSLQMDNLKPEDTAVYYCHVLEDRVDSFHDY |
| | | | WGQGTQVTVSSGAGSSELIKENMHMKLYMEGTVDNHH |
| | | | FKCTSEGEGKPYEGTQTMRIKVVEGGPLPFAFDILATSFL |
| | | | YGSKTFINHTQGIPDFFKQSFPEGFTWERVTTYEDGGVLT |
| | | | ATQDTSLQDGCLIYNVKIRGVNFTSNGPVMQKKTLGWE |
| | | | AFTETLYPADGGLEGRNDMALKLVGGSHLIANIKTTYRS |
| | | | KKPAKNLKMPGVYYVDYRLERIKEANNETYVEQHEVAV |
| | | | ARYCDLPSKLGHKLN* |
| MCP-linker- | 29 | 4C, 4E | MASNFTQFVLVDNGGTGDVTVAPSNFANGIAEWISSNSR |
| ADAR2- | | | SQAYKVTCSVRQSSAQNRKYTIKVEVPKGAWRSYLNME |
| DDN- | | | LTIPIFATNSDCELIVKAMQGLLKDGNPIPSAIAANSGIYG |
| ALFA-78- | | | GSGSGAGSGSPAGGGAPGSGGGSQLHLPQVLADAVSRLV |
| ADAR2 | | | LGKFGDLTDNFSSPHARRKVLAGVVMTTGTDVKDAKVI |
| (E488Q)-DDC- | | | SVSTGTKCINGEYMSDRGLALNDCHAEIISRRSLLRFLYT |
| TagBFP | | | QLELYLNNKDDQKRSIFQKSERGGFRLKENVQFHLYISTS |
| | | | PCGDARIFSPHEPILEEPAASGSGPGRLEQEIRARLSPGTG |
| | | | DRHPNRKARGQLRTKIESGQGTIPVRSNASIQTWDGVLQ |
| | | | GERLLTMSCSDKIARWNVVGIQGSLLSIFVEPIYFSSIILGS |
| | | | LYHGDHLSRAMYQRISNIEDLPPLYTLNKPLLSGISNAEA |
| | | | RQPGKAPNFSVNWTVGDSAIEVINATTGKDELGRASRLC |
| | | | KHALYCRWMRVHGKVPSHLLRSKITKPNVYHESKLAAK |
| | | | EYQAAKARLFTAFIKAGLGAWVEKPTEQDQFSLTGSGSS |
| | | | ELIKENMHMKLYMEGTVDNHHFKCTSEGEGKPYEGTQT |
| | | | MRIKVVEGGPLPFAFDILATSFLYGSKTFINHTQGIPDFFK |
| | | | QSFPEGFTWERVTTYEDGGVLTATQDTSLQDGCLIYNVK |
| | | | IRGVNFTSNGPVMQKKTLGWEAFTETLYPADGGLEGRN |
| | | | DMALKLVGGSHLIANIKTTYRSKKPAKNLKMPGVYYVD |
| | | | YRLERIKEANNETYVEQHEVAVARYCDLPSKLGHKLN* |
| MCP-linker- | 30 | 4C, 4E | MASNFTQFVLVDNGGTGDVTVAPSNFANGIAEWISSNSR |
| ADAR2- | | | SQAYKVTCSVRQSSAQNRKYTIKVEVPKGAWRSYLNME |
| DDN- | | | LTIPIFATNSDCELIVKAMQGLLKDGNPIPSAIAANSGIYG |
| ALFA-PE- | | | GSGSGAGSGSPAGGGAPGSGGGSQLHLPQVLADAVSRLV |
| ADAR2 | | | LGKFGDLTDNFSSPHARRKVLAGVVMTTGTDVKDAKVI |
| (E488Q)-DDC- | | | SVSTGTKCINGEYMSDRGLALNDCHAEIISRRSLLRFLYT |
| NbALFA- | | | QLELYLNNKDDQKRSIFQKSERGGFRLKENVQFHLYISTS |
| TagBFP | | | PCGDARIFSPHEPILEEPAASGSGPGRLEQEIRARLSPGTG |
| | | | DRHPNRKARGQLRTKIESGQGTIPVRSNASIQTWDGVLQ |
| | | | GERLLTMSCSDKIARWNVVGIQGSLLSIFVEPIYFSSIILGS |
| | | | LYHGDHLSRAMYQRISNIEDLPPLYTLNKPLLSGISNAEA |
| | | | RQPGKAPNFSVNWTVGDSAIEVINATTGKDELGRASRLC |
| | | | KHALYCRWMRVHGKVPSHLLRSKITKPNVYHESKLAAK |
| | | | EYQAAKARLFTAFIKAGLGAWVEKPTEQDQFSLTGSGGT |
| | | | AEVQLQESGGGLVQPGGSLRLSCTASGVTISALNAMAMG |
| | | | WYRQAPGERRVMVAAVSERGNAMYRESVQGRFTVTRD |
| | | | FTNKMVSLQMDNLKPEDTAVYYCHVLEDRVDSFHDYW |
| | | | GQGTQVTVSSGAGSSELIKENMHMKLYMEGTVDNHHFK |
| | | | CTSEGEGKPYEGTQTMRIKVVEGGPLPFAFDILATSFLYG |
| | | | SKTFINHTQGIPDFFKQSFPEGFTWERVTTYEDGGVLTAT |
| | | | QDTSLQDGCLIYNVKIRGVNFTSNGPVMQKKTLGWEAFT |
| | | | ETLYPADGGLEGRNDMALKLVGGSHLIANIKTTYRSKKP |
| | | | AKNLKMPGVYYVDYRLERIKEANNETYVEQHEVAVARY |
| | | | CDLPSKLGHKLN* |
| miRFP670- | 31 | 4C-E | MVAGHASGSPAFGTASHSNCEHEEIHLAGSIQPHGALLV |
| ALFA | | | VSEHDHRVIQASANAAEFLNLGSVLGVPLAEIDGDLLIKI |
| | | | LPHLDPTAEGMPVAVRCRIGNPSTEYCGLMHRPPEGGLII |
| | | | ELERAGPSIDLSGTLAPALERIRTAGSLRALCDDTVLLFQQ |
| | | | CTGYDRVMVYRFDEQGHGLVFSECHVPGLESYFGNRYP |
| | | | SSTVPQMARQLYVRQRVRVLVDVTYQPVPLEPRLSPLTG |
| | | | RDLDMSGCFLRSMSPCHLQFLKDMGVRATLAVSLVVGG |
| | | | KLWGLVVCHHYLPRFIRFELRAICKRLAERIATRITALESL |
| | | | ESRLEEELRRRLTE* |
| MCP-linker- | 32 | 5D | MASNFTQFVLVDNGGTGDVTVAPSNFANGIAEWISSNSR |
| ADAR2- | | | SQAYKVTCSVRQSSAQNRKYTIKVEVPKGAWRSYLNME |
| DDN- | | | LTIPIFATNSDCELIVKAMQGLLKDGNPIPSAIAANSGIYG |
| SpyTag- | | | GSGSGAGSGSPAGGGAPGSGGGSQLHLPQVLADAVSRLV |
| ADAR2 | | | LGKFGDLTDNFSSPHARRKVLAGVVMTTGTDVKDAKVI |
| (E488Q)-DDC- | | | SVSTGTKCINGEYMSDRGLALNDCHAEIISRRSLLRFLYT |
| TagBFP | | | QLELYLNNKDDQKRSIFQKSERGGFRLKENVQFHLYISTS |
| | | | PCGDARIFSPHEPILEEPAASGGSGAHIVMVDAYKPTKGT |
| | | | GDRHPNRKARGQLRTKIESGQGTIPVRSNASIQTWDGVL |
| | | | QGERLLTMSCSDKIARWNVVGIQGSLLSIFVEPIYFSSIILG |
| | | | SLYHGDHLSRAMYQRISNIEDLPPLYTLNKPLLSGISNAE |
| | | | ARQPGKAPNFSVNWTVGDSAIEVINATTGKDELGRASRL |
| | | | CKHALYCRWMRVHGKVPSHLLRSKITKPNVYHESKLAA |
| | | | KEYQAAKARLFTAFIKAGLGAWVEKPTEQDQFSLTGSGS |
| | | | SELIKENMHMKLYMEGTVDNHHFKCTSEGEGKPYEGTQ |
| | | | TMRIKVVEGGPLPFAFDILATSFLYGSKTFINHTQGIPDFF |
| | | | KQSFPEGFTWERVTTYEDGGVLTATQDTSLQDGCLIYNV |
| | | | KIRGVNFTSNGPVMQKKTLGWEAFTETLYPADGGLEGR |
| | | | NDMALKLVGGSHLIANIKTTYRSKKPAKNLKMPGVYYV |
| | | | DYRLERIKEANNETYVEQHEVAVARYCDLPSKLGHKLN* |
| MCP-linker- | 33 | 5D | MASNFTQFVLVDNGGTGDVTVAPSNFANGIAEWISSNSR |
| ADAR2- | | | SQAYKVTCSVRQSSAQNRKYTIKVEVPKGAWRSYLNME |
| DDN- | | | LTIPIFATNSDCELIVKAMQGLLKDGNPIPSAIAANSGIYG |
| SpyTag- | | | GSGSGAGSGSPAGGGAPGSGGGSQLHLPQVLADAVSRLV |
| ADAR2 | | | LGKFGDLTDNFSSPHARRKVLAGVVMTTGTDVKDAKVI |
| (E488Q)-DDC- | | | SVSTGTKCINGEYMSDRGLALNDCHAEIISRRSLLRFLYT |
| SpyCatcher- | | | QLELYLNNKDDQKRSIFQKSERGGFRLKENVQFHLYISTS |
| TagBFP | | | PCGDARIFSPHEPILEEPAASGGSGAHIVMVDAYKPTKGT |
| | | | GDRHPNRKARGQLRTKIESGQGTIPVRSNASIQTWDGVL |
| | | | QGERLLTMSCSDKIARWNVVGIQGSLLSIFVEPIYFSSIILG |
| | | | SLYHGDHLSRAMYQRISNIEDLPPLYTLNKPLLSGISNAE |
| | | | ARQPGKAPNFSVNWTVGDSAIEVINATTGKDELGRASRL |
| | | | CKHALYCRWMRVHGKVPSHLLRSKITKPNVYHESKLAA |
| | | | KEYQAAKARLFTAFIKAGLGAWVEKPTEQDQFSLTGSGT |
| | | | SGGAMVDTLSGLSSEQGQSGDMTIEEDSATHIKFSKRDE |
| | | | DGKELAGATMELRDSSGKTISTWISDGQVKDFYLYPGKY |
| | | | TFVETAAPDGYEVATAITFTVNEQGQVTVNGKATKGDA |
| | | | HIGSSELIKENMHMKLYMEGTVDNHHFKCTSEGEGKPYE |
| | | | GTQTMRIKVVEGGPLPFAFDILATSFLYGSKTFINHTQGIP |
| | | | DFFKQSFPEGFTWERVTTYEDGGVLTATQDTSLQDGCLI |
| | | | YNVKIRGVNFTSNGPVMQKKTLGWEAFTETLYPADGGL |
| | | | EGRNDMALKLVGGSHLIANIKTTYRSKKPAKNLKMPGV |
| | | | YYVDYRLERIKEANNETYVEQHEVAVARYCDLPSKLGH |
| | | | KLN* |
| MCP-linker- | 34 | 5D | MASNFTQFVLVDNGGTGDVTVAPSNFANGIAEWISSNSR |
| ADAR2- | | | SQAYKVTCSVRQSSAQNRKYTIKVEVPKGAWRSYLNME |
| DDN- | | | LTIPIFATNSDCELIVKAMQGLLKDGNPIPSAIAANSGIYG |
| Spy Tag- | | | GSGSGAGSGSPAGGGAPGSGGGSQLHLPQVLADAVSRLV |
| ADAR2 | | | LGKFGDLTDNFSSPHARRKVLAGVVMTTGTDVKDAKVI |
| (E488Q)-DDC- | | | SVSTGTKCINGEYMSDRGLALNDCHAEIISRRSLLRFLYT |
| TagBFP - | | | QLELYLNNKDDQKRSIFQKSERGGFRLKENVQFHLYISTS |
| P2A-T2A- | | | PCGDARIFSPHEPILEEPAASGGSGAHIVMVDAYKPTKGT |
| SpyCatcher | | | GDRHPNRKARGQLRTKIESGQGTIPVRSNASIQTWDGVL |
| | | | QGERLLTMSCSDKIARWNVVGIQGSLLSIFVEPIYFSSIILG |
| | | | SLYHGDHLSRAMYQRISNIEDLPPLYTLNKPLLSGISNAE |
| | | | ARQPGKAPNFSVNWTVGDSAIEVINATTGKDELGRASRL |
| | | | CKHALYCRWMRVHGKVPSHLLRSKITKPNVYHESKLAA |
| | | | KEYQAAKARLFTAFIKAGLGAWVEKPTEQDQFSLTGSGS |
| | | | SELIKENMHMKLYMEGTVDNHHFKCTSEGEGKPYEGTQ |
| | | | TMRIKVVEGGPLPFAFDILATSFLYGSKTFINHTQGIPDFF |
| | | | KQSFPEGFTWERVTTYEDGGVLTATQDTSLQDGCLIYNV |
| | | | KIRGVNFTSNGPVMQKKTLGWEAFTETLYPADGGLEGR |
| | | | NDMALKLVGGSHLIANIKTTYRSKKPAKNLKMPGVYYV |
| | | | DYRLERIKEANNETYVEQHEVAVARYCDLPSKLGHKLNT |
| | | | SATNFSLLKQAGDVEENPGPGGSEGRGSLLTCGDVEENP |
| | | | GPGTSGGAMVDTLSGLSSEQGQSGDMTIEEDSATHIKFSK |
| | | | RDEDGKELAGATMELRDSSGKTISTWISDGQVKDFYLYP |
| | | | GKYTFVETAAPDGYEVATAITFTVNEQGQVTVNGKATK |
| | | | GDAHIG* |
| MCP-linker- | 35 | 5D-E | MASNFTQFVLVDNGGTGDVTVAPSNFANGIAEWISSNSR |
| ADAR2- | | | SQAYKVTCSVRQSSAQNRKYTIKVEVPKGAWRSYLNME |
| DDN- | | | LTIPIFATNSDCELIVKAMQGLLKDGNPIPSAIAANSGIYG |
| Spy Tag- | | | GSGSGAGSGSPAGGGAPGSGGGSQLHLPQVLADAVSRLV |
| ADAR2 | | | LGKFGDLTDNFSSPHARRKVLAGVVMTTGTDVKDAKVI |
| (E488Q)-DDC- | | | SVSTGTKCINGEYMSDRGLALNDCHAEIISRRSLLRFLYT |
| TEVcs- | | | QLELYLNNKDDQKRSIFQKSERGGFRLKENVQFHLYISTS |
| SpyCatcher- | | | PCGDARIFSPHEPILEEPAASGGSGAHIVMVDAYKPTKGT |
| TagBFP | | | GDRHPNRKARGQLRTKIESGQGTIPVRSNASIQTWDGVL |
| | | | QGERLLTMSCSDKIARWNVVGIQGSLLSIFVEPIYFSSIILG |
| | | | SLYHGDHLSRAMYQRISNIEDLPPLYTLNKPLLSGISNAE |
| | | | ARQPGKAPNFSVNWTVGDSAIEVINATTGKDELGRASRL |
| | | | CKHALYCRWMRVHGKVPSHLLRSKITKPNVYHESKLAA |
| | | | KEYQAAKARLFTAFIKAGLGAWVEKPTEQDQFSLTGSGG |
| | | | TENLYFQSGTSGGAMVDTLSGLSSEQGQSGDMTIEEDSA |
| | | | THIKFSKRDEDGKELAGATMELRDSSGKTISTWISDGQVK |
| | | | DFYLYPGKYTFVETAAPDGYEVATAITFTVNEQGQVTVN |
| | | | GKATKGDAHIGSSELIKENMHMKLYMEGTVDNHHFKCT |
| | | | SEGEGKPYEGTQTMRIKVVEGGPLPFAFDILATSFLYGSK |
| | | | TFINHTQGIPDFFKQSFPEGFTWERVTTYEDGGVLTATQD |
| | | | TSLQDGCLIYNVKIRGVNFTSNGPVMQKKTLGWEAFTET |
| | | | LYPADGGLEGRNDMALKLVGGSHLIANIKTTYRSKKPAK |
| | | | NLKMPGVYYVDYRLERIKEANNETYVEQHEVAVARYCD |
| | | | LPSKLGHKLN* |
| TEV | 36 | 5D-E | MGESLFKGPRDYNPISSTICHLTNESDGHTTSLYGIGFGPFI |
| Protease | | | ITNKHLFRRNNGTLLVQSLHGVFKVKNTTTLQQHLIDGR |
| | | | DMIIIRMPKDFPPFPQKLKFREPQREERICLVTTNFQTKSM |
| | | | SSMVSDTSCTFPSSDGIFWKHWIQTKDGQCGSPLVSTRDG |
| | | | FIVGIHSASNFTNTNNYFTSVPKNFMELLTNQEAQQWVS |
| | | | GWRLNADSVLWGGHKVFMVKPEEPFQPVKEATQL* |
| MCP-linker- | 37 | 5G | MASNFTQFVLVDNGGTGDVTVAPSNFANGIAEWISSNSR |
| ADAR2- | | | SQAYKVTCSVRQSSAQNRKYTIKVEVPKGAWRSYLNME |
| DDN- | | | LTIPIFATNSDCELIVKAMQGLLKDGNPIPSAIAANSGIYG |
| Bad(L)- | | | GSGSGAGSGSPAGGGAPGSGGGSQLHLPQVLADAVSRLV |
| ADAR2 | | | LGKFGDLTDNFSSPHARRKVLAGVVMTTGTDVKDAKVI |
| (E488Q)-DDC- | | | SVSTGTKCINGEYMSDRGLALNDCHAEIISRRSLLRFLYT |
| PhoCl-Bcl- | | | QLELYLNNKDDQKRSIFQKSERGGFRLKENVQFHLYISTS |
| xL-TagBFP | | | PCGDARIFSPHEPILEEPAASGSGTGAPPNLWAAQRYGRE |
| | | | LRRMSDELVDRHPNRKARGQLRTKIESGQGTIPVRSNASI |
| | | | QTWDGVLQGERLLTMSCSDKIARWNVVGIQGSLLSIFVE |
| | | | PIYFSSIILGSLYHGDHLSRAMYQRISNIEDLPPLYTLNKPL |
| | | | LSGISNAEARQPGKAPNFSVNWTVGDSAIEVINATTGKDE |
| | | | LGRASRLCKHALYCRWMRVHGKVPSHLLRSKITKPNVY |
| | | | HESKLAAKEYQAAKARLFTAFIKAGLGAWVEKPTEQDQ |
| | | | FSLTGSGSGGVIPDYFKQSFPEGYSWERSMTYEDGGICIA |
| | | | TNDITMEGDSFINKIHFKGTNFPPNGPVMQKRTVGWEAS |
| | | | TEKMYERDGVLKGDVKMKLLLKGGGHYRCDYRTTYKV |
| | | | KQKPVKLPDYHFVDHRIEILSHDKDYNKVKLYEHAVAR |
| | | | NSTDSMDELYKGGSGGMVSKGEETITSVIKPDMKNKLR |
| | | | MEGNVNGHAFVIEGEGSGKPFEGIQTIDLEVKEGAPLPFA |
| | | | YDILTTAFHYGNRVFTKYPRSGSGSSNRELVVDFLSYKLS |
| | | | QKGYSWSQFSDVEENRTEAPEGTESEMETPSAINGNPSW |
| | | | HLADSPAVNGATGHSSSLDAREVIPMAAVKQALREAGD |
| | | | EFELRYRRAFSDLTSQLHITPGTAYQSFEQVVNELFRDGV |
| | | | NWGRIVAFFSFGGALCVESVDKEMQVLVSRIAAWMATY |
| | | | LNDHLEPWIQENGGWDTFVELYGNNGSSELIKENMHMK |
| | | | LYMEGTVDNHHFKCTSEGEGKPYEGTQTMRIKVVEGGP |
| | | | LPFAFDILATSFLYGSKTFINHTQGIPDFFKQSFPEGFTWER |
| | | | VTTYEDGGVLTATQDTSLQDGCLIYNVKIRGVNFTSNGP |
| | | | VMQKKTLGWEAFTETLYPADGGLEGRNDMALKLVGGS |
| | | | HLIANIKTTYRSKKPAKNLKMPGVYYVDYRLERIKEANN |
| | | | ETYVEQHEVAVARYCDLPSKLGHKLN* |

### Example 4

### DNA Sequences of Relevant Plasmids (Sequences Given are Between Promoter and Terminator):

RNA features are highlighted accordingly.

SEQ ID NO: 38 (see e.g., polypeptide in SEQ ID NO: 1) - mCherry-FLAG-P2A-T2A-UAG-
UAG-MS2-P2A-T2A-HA-mNeonGreen; UAG-UAG is bolded; MS2 is bold italicized.
AGGTAAGCTTGGTACCGAGCTCGGATCCaccggtcgccaccatggtgagcaagggcgaggaggataacatggccatcatca
aggagttcatgcgcttcaaggtgcacatggagggctccgtgaacggccacgagttcgagatcgagggcgagggcgagggccgcccctacgagggc
acccagaccgccaagctgaaggtgaccaagggtggccccctgcccttcgcctgggacatcctgtcccctcagttcatgtacggctccaaggcctacgtg
aagcaccccgccgacatccccgactacttgaagctgtccttccccgagggcttcaagtgggagcgcgtgatgaacttcgaggacggcggcgtggtgac
cgtgacccaggactcctccctgcaggacggcgagttcatctacaaggtgaagctgcgcggcaccaacttcccctccgacggccccgtaatgcagaaga
agaccatgggctgggaggcctcctccgagcggatgtaccccgaggacggcgccctgaagggcgagatcaagcagaggctgaagctgaaggacgg
cggccactacgacgctgaggtcaagaccacctacaaggccaagaagcccgtgcagctgcccggcgcctacaacgtcaacatcaagttggacatcacc
tcccacaacgaggactacaccatcgtggaacagtacgaacgcgccgagggccgccactccaccggcggcatggacgagctgTACaaggattaca
aggatgacgatgacaaaGGTAGCGGGGCAACTAATTTTAGCTTACTCAAACAGGCTGGGGACGTCGA
GGAGAATCCAGGCCCTGCATCCGCTGGCTCTGGAGAAGGACGAGGCTCCTTGCTCACCTGTG
GAGATGTCGAAGAGAACCCAGGTCCTGCAACCGGGAATTCCGCGTAGCGCTAGCTTTGCC
AGCGCCACGCGaaACATGAGGATcACCCATGTACTAGTGCCACAAACTTCTCTCTGCTAAA
GCAAGCAGGTGATGTTGAAGAAAACCCAGGGCCTGGAGGGTCCGAGGGCAGGGGAAGTCTC
CTAACATGCGGGGACGTGGAGGAAAATCCCGGCCCATCCGGATATCCCTACGATGTGCCCG
ATTACGCTCATatggtgagcaagggcgaggaggataacatggcctctctcccagcgacacatgagttacacatctttggctccatcaacggtgt
ggactttgacatggtgggtcagggcaccggcaatccaaatgatggttatgaggagttaaacctgaagtccaccaagggtgacctccagttctccccctgg
attctggtccctcatatcgggtatggcttccatcagtacctgccctaccctgacgggatgtcgcctttccaggccgccatggtagatggcAGCggatacc
aagtccatcgcacaatgcagtttgaagatggtgcctcccttactgttaactaccgctacacctacgagggaagccacatcaaaggagaggcccaggtga
aggggactggtttccctgctgacggtcctgtgatgaccaactcgctgaccgctgcggactggtgcaggtcgaagaagacttaccccaacgacaaaacca
tcatcagtacctttaagtggagttacaccactggaaatggcaagAGAtaccggagcactgcgcggaccacctacacctttgccaagccaatggcggct
aactatctgaagaaccagccgatgtacgtgttccgtaagacggagctcaagcactccaagaccgagctcaacttcaaggagtggcaaaaggcctttacc
gatgtgatgGGAatggacGAGCTGTATaagGCTAGCTAAGCGGCCGCTCGAGTCTAGAGGGCCCGCGG
TTCGAAGGTA
SEQ ID NO: 39 (see e.g., polypeptide in SEQ ID NO: 2) - MCP-linker-ADAR2(E488Q)-
TagBFP
gatcgaacccttaaggccaccatggcgtccaatttcactcagtttgtgctggttgacaacggcgggaccggggacgttacggtagccccctcaaactttgc
caacggtatagcggagtggataagcagcaattctaggagtcaagcatacaaagttacatgcagcgtgcgccaatctagcgctcagaatcgcaagtacac
cattaaagtagaggtccccaagggagcctggagaagctatcttaacatggagttgaccataccaatcttcgctaccaactctgactgtgaactcattgtgaa
agccatgcaaggtctgctcaaggatggtaacccaattccgtccgctatcgctgccaactctgggatttacgggggcagtgggagcggtgcaggatctgg
tagtccagctgggggaggagcaccgggtagcggtggggggtctcagctgcacctgccccaggttctcgcagacgccgtatcccgccttgtactgggca
agtttggtgatcttactgacaatttttcatctcctcatgcgaggcggaaagtactcgcaggcgtcgtcatgacgaccggaactgacgtgaaagacgccaaa
gtcatctctgtctccacgggcacaaagtgcataaacggggagtacatgagcgaccgggggctggcactgaatgattgtcacgctgaaataatatctaggc
gatctctgcttagatttctctacactcaactcgaattgtaccttaacaacaaagatgaccagaaacgcagtatatttcagaaatcagaacgcggcggatttcg
acttaaggaaaacgttcagttccacttgtatatcagcacatccccttgcggtgacgcccgaatcttttccccgcacgagccgatattggaggagcccgcgg
acagacatcctaataggaaggctagaggccaacttcggacgaagattgaaagtggccagggtactatcccggtgcggtccaacgctagtattcaaacgt
gggacggagtccttcaaggtgaacggctgttgacaatgagctgctcagacaaaatcgcgcgctggaatgtagtgggaatccaaggcagcctcttgagc
atattcgtagaacccatatatttctcatccattattttgggctctctgtatcatggtgaccatctgtcaagggctatgtaccaacgaatttctaatatcgaggatctt
cctccactctatacactcaataagcctctcttgtccgggatatcaaacgctgaggcccgccagccagggaaagctcctaacttcagtgttaactggaccgtt
ggtgattctgcgatagaggtcatcaacgccacgacaggtaaggatgagctcggtagagcctcacgcctgtgtaaacacgcgttgtattgtagatggatga
gagtacatgggaaggtcccatctcacttgctccgaagcaagatcactaagcctaatgtgtatcatgagtcaaaactcgcggctaaagaataccaggcagc
caaagctcgactttttacagcttttattaaggcagggctcggggcatgggtcgagaagccgaccgagcaggaccaattctctctgacggggagcggatc
cAGCGAGCTGATTAAGGAGAACATGCACATGAAGCTGTACATGGAGGGCACCGTGGACAAC
CATCACTTCAAGTGCACATCCGAGGGCGAAGGCAAGCCCTACGAGGGCACCCAGACCATGA
GAATCAAGGTGGTCGAGGGCGGCCCTCTCCCCTTCGCCTTCGACATCCTGGCTACTAGCTTC
CTCTACGGCAGCAAGACCTTCATCAACCACACCCAGGGCATCCCCGACTTCTTCAAGCAGTC
CTTCCCTGAGGGCTTCACATGGGAGAGAGTCACCACATACGAAGACGGGGGCGTGCTGACC
GCTACCCAGGACACCAGCCTCCAGGACGGCTGCCTCATCTACAACGTCAAGATCAGAGGGG
TGAACTTCACATCCAACGGCCCTGTGATGCAGAAGAAAACACTCGGCTGGGAGGCCTTCACC
GAGACGCTGTACCCCGCTGACGGCGGCCTGGAAGGCAGAAACGACATGGCCCTGAAGCTCG
TGGGCGGGAGCCATCTGATCGCAAACATCAAGACCACATATAGATCCAAGAAACCCGCTAA
GAACCTCAAGATGCCTGGCGTCTACTATGTGGACTACAGACTGGAAAGAATCAAGGAGGCC
AACAACGAGACCTACGTCGAGCAGCACGAGGTGGCAGTGGCCAGATACTGCGACCTCCCTA
GCAAACTGGGGCACAAGCTTAATtAAGGGCCCGTTTAAACCCGC
SEQ ID NO: 40 (see e.g., polypeptide in SEQ ID NO: 3) - MCP-linker-ADAR2(E396A &
E488Q)-TagBFP
TAATACGACTCACTATAGGGAGACCCAAGCTGGctagaggatcgaacccttaaggccaccatggcgtccaatttcactca
gtttgtgctggttgacaacggcgggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctGCCataatatctaggcgatctctgcttagatttctctacactcaactcgaattgta
ccttaacaacaaagatgaccagaaacgcagtatatttcagaaatcagaacgcggcggatttcgacttaaggaaaacgttcagttccacttgtatatcagcac
atccccttgcggtgacgcccgaatcttttccccgcacgagccgatattggaggagcccgcggacagacatcctaataggaaggctagaggccaacttcg
gacgaagattgaaagtggccagggtactatcccggtgcggtccaacgctagtattcaaacgtgggacggagtccttcaaggtgaacggctgttgacaat
gagctgctcagacaaaatcgcgcgctggaatgtagtgggaatccaaggcagcctcttgagcatattcgtagaacccatatatttctcatccattattttgggc
tctctgtatcatggtgaccatctgtcaagggctatgtaccaacgaatttctaatatcgaggatcttcctccactctatacactcaataagcctctcttgtccggg
atatcaaacgctgaggcccgccagccagggaaagctcctaacttcagtgttaactggaccgttggtgattctgcgatagaggtcatcaacgccacgacag
gtaaggatgagctcggtagagcctcacgcctgtgtaaacacgcgttgtattgtagatggatgagagtacatgggaaggtcccatctcacttgctccgaagc
aagatcactaagcctaatgtgtatcatgagtcaaaactcgcggctaaagaataccaggcagccaaagctcgactttttacagcttttattaaggcagggctc
ggggcatgggtcgagaagccgaccgagcaggaccaattctctctgacggggagcggatccAGCGAGCTGATTAAGGAGAACA
TGCACATGAAGCTGTACATGGAGGGCACCGTGGACAACCATCACTTCAAGTGCACATCCGA
GGGCGAAGGCAAGCCCTACGAGGGCACCCAGACCATGAGAATCAAGGTGGTCGAGGGCGG
CCCTCTCCCCTTCGCCTTCGACATCCTGGCTACTAGCTTCCTCTACGGCAGCAAGACCTTCAT
CAACCACACCCAGGGCATCCCCGACTTCTTCAAGCAGTCCTTCCCTGAGGGCTTCACATGGG
AGAGAGTCACCACATACGAAGACGGGGGCGTGCTGACCGCTACCCAGGACACCAGCCTCCA
GGACGGCTGCCTCATCTACAACGTCAAGATCAGAGGGGTGAACTTCACATCCAACGGCCCTG
TGATGCAGAAGAAAACACTCGGCTGGGAGGCCTTCACCGAGACGCTGTACCCCGCTGACGG
CGGCCTGGAAGGCAGAAACGACATGGCCCTGAAGCTCGTGGGCGGGAGCCATCTGATCGCA
AACATCAAGACCACATATAGATCCAAGAAACCCGCTAAGAACCTCAAGATGCCTGGCGTCT
ACTATGTGGACTACAGACTGGAAAGAATCAAGGAGGCCAACAACGAGACCTACGTCGAGCA
GCACGAGGTGGCAGTGGCCAGATACTGCGACCTCCCTAGCAAACTGGGGCACAAGCTTAATt
AAGGGCCCGTTTAAACCCGCTGATCAGCCTCGACTGTGCCTTCTA
SEQ ID NO: 41 (see e.g., polypeptide in SEQ ID NO: 4) - mCherry-FLAG-UGG-UGG-P2A-
T2A-HA-mNeonGreen; UGG-UGG is bolded
TAATACGACTCACTATAGGGAGACCCAAGCTGGCTAGGTAAGCTTGGTACCGAGCTCGGATC
Caccggtcgccaccatggtgagcaagggcgaggaggataacatggccatcatcaaggagttcatgcgcttcaaggtgcacatggagggctccgtgaa
cggccacgagttcgagatcgagggcgagggcgagggccgcccctacgagggcacccagaccgccaagctgaaggtgaccaagggtggccccctg
cccttcgcctgggacatcctgtcccctcagttcatgtacggctccaaggcctacgtgaagcaccccgccgacatccccgactacttgaagctgtccttccc
cgagggcttcaagtgggagcgcgtgatgaacttcgaggacggcggcgtggtgaccgtgacccaggactcctccctgcaggacggcgagttcatctac
aaggtgaagctgcgcggcaccaacttcccctccgacggccccgtaatgcagaagaagaccatgggctgggaggcctcctccgagcggatgtacccc
gaggacggcgccctgaagggcgagatcaagcagaggctgaagctgaaggacggcggccactacgacgctgaggtcaagaccacctacaaggcca
agaagcccgtgcagctgcccggcgcctacaacgtcaacatcaagttggacatcacctcccacaacgaggactacaccatcgtggaacagtacgaacg
cgccgagggccgccactccaccggcggcatggacgagctgTACaaggattacaaggatgacgatgacaagAATTCCGCGTGGCG
CTGGCTTCCTTGCCAGCGCCACGCGACTAGTGCCACAAACTTCTCTCTGCTAAAGCAAGC
AGGTGATGTTGAAGAAAACCCAGGGCCTGGAGGGTCCGAGGGCAGGGGAAGTCTCCTAACA
TGCGGGGACGTGGAGGAAAATCCCGGCCCATCCGGATATCCCTACGATGTGCCCGATTACGC
TCATatggtgagcaagggcgaggaggataacatggcctctctcccagcgacacatgagttacacatctttggctccatcaacggtgtggactttgaca
tggtgggtcagggcaccggcaatccaaatgatggttatgaggagttaaacctgaagtccaccaagggtgacctccagttctccccctggattctggtccct
catatcgggtatggcttccatcagtacctgccctaccctgacgggatgtcgcctttccaggccgccatggtagatggcAGCggataccaagtccatcgc
acaatgcagtttgaagatggtgcctcccttactgttaactaccgctacacctacgagggaagccacatcaaaggagaggcccaggtgaaggggactggt
ttccctgctgacggtcctgtgatgaccaactcgctgaccgctgcggactggtgcaggtcgaagaagacttaccccaacgacaaaaccatcatcagtacct
ttaagtggagttacaccactggaaatggcaagAGAtaccggagcactgcgcggaccacctacacctttgccaagccaatggcggctaactatctgaa
gaaccagccgatgtacgtgttccgtaagacggagctcaagcactccaagaccgagctcaacttcaaggagtggcaaaaggcctttaccgatgtgatgG
GAatggacGAGCTGTATaagGCTAGCTAAGCGGCCGCTCGAGTCTAGAGGGCCCGCGGTTCGAA
GGTAAGCCTATCCCTAACCCTCTCCTCGGTCTCGATTCTACGCGTACCGGTCATCATCACCAT
CACCATTGAGTTTAAACCCGCTGATCAGCCTCGACTGTGCCTTCTA
SEQ ID NO: 42 (see e.g., polypeptide in SEQ ID NO: 5) - mCherry-FLAG-UAG-UGG-P2A-
T2A-HA-mNeonGreen; UAG-UGG is bolded
TAATACGACTCACTATAGGGAGACCCAAGCTGGCTAGGTAAGCTTGGTACCGAGCTCGGATC
Caccggtcgccaccatggtgagcaagggcgaggaggataacatggccatcatcaaggagttcatgcgcttcaaggtgcacatggagggctccgtgaa
cggccacgagttcgagatcgagggcgagggcgagggccgcccctacgagggcacccagaccgccaagctgaaggtgaccaagggtggccccctg
cccttcgcctgggacatcctgtcccctcagttcatgtacggctccaaggcctacgtgaagcaccccgccgacatccccgactacttgaagctgtccttccc
cgagggcttcaagtgggagcgcgtgatgaacttcgaggacggcggcgtggtgaccgtgacccaggactcctccctgcaggacggcgagttcatctac
aaggtgaagctgcgcggcaccaacttcccctccgacggccccgtaatgcagaagaagaccatgggctgggaggcctcctccgagcggatgtacccc
gaggacggcgccctgaagggcgagatcaagcagaggctgaagctgaaggacggcggccactacgacgctgaggtcaagaccacctacaaggcca
agaagcccgtgcagctgcccggcgcctacaacgtcaacatcaagttggacatcacctcccacaacgaggactacaccatcgtggaacagtacgaacg
cgccgagggccgccactccaccggcggcatggacgagctgTACaaggattacaaggatgacgatgacaagAATTCCGCGTAGCG
CTGGCTTCCTTGCCAGCGCCACGCGACTAGTGCCACAAACTTCTCTCTGCTAAAGCAAGC
AGGTGATGTTGAAGAAAACCCAGGGCCTGGAGGGTCCGAGGGCAGGGGAAGTCTCCTAACA
TGCGGGGACGTGGAGGAAAATCCCGGCCCATCCGGATATCCCTACGATGTGCCCGATTACGC
TCATatggtgagcaagggcgaggaggataacatggcctctctcccagcgacacatgagttacacatctttggctccatcaacggtgtggactttgaca
tggtgggtcagggcaccggcaatccaaatgatggttatgaggagttaaacctgaagtccaccaagggtgacctccagttctccccctggattctggtccct
catatcgggtatggcttccatcagtacctgccctaccctgacgggatgtcgcctttccaggccgccatggtagatggcAGCggataccaagtccatcgc
acaatgcagtttgaagatggtgcctcccttactgttaactaccgctacacctacgagggaagccacatcaaaggagaggcccaggtgaaggggactggt
ttccctgctgacggtcctgtgatgaccaactcgctgaccgctgcggactggtgcaggtcgaagaagacttaccccaacgacaaaaccatcatcagtacct
ttaagtggagttacaccactggaaatggcaagAGAtaccggagcactgcgcggaccacctacacctttgccaagccaatggcggctaactatctgaa
gaaccagccgatgtacgtgttccgtaagacggagctcaagcactccaagaccgagctcaacttcaaggagtggcaaaaggcctttaccgatgtgatgG
GAatggacGAGCTGTATaagGCTAGCTAAGCGGCCGCTCGAGTCTAGAGGGCCCGCGGTTCGAA
GGTAAGCCTATCCCTAACCCTCTCCTCGGTCTCGATTCTACGCGTACCGGTCATCATCACCAT
CACCATTGAGTTTAAACCCGCTGATCAGCCTCGACTGTGCCTTCTA
SEQ ID NO: 43 (see e.g., polypeptide in SEQ ID NO: 6) - mCherry-FLAG-UAG-UAG-P2A-
T2A-HA-mNeonGreen; UAG-UAG is bolded
TAATACGACTCACTATAGGGAGACCCAAGCTGGCTAGGTAAGCTTGGTACCGAGCTCGGATC
Caccggtcgccaccatggtgagcaagggcgaggaggataacatggccatcatcaaggagttcatgcgcttcaaggtgcacatggagggctccgtgaa
cggccacgagttcgagatcgagggcgagggcgagggccgcccctacgagggcacccagaccgccaagctgaaggtgaccaagggtggccccctg
cccttcgcctgggacatcctgtcccctcagttcatgtacggctccaaggcctacgtgaagcaccccgccgacatccccgactacttgaagctgtccttccc
cgagggcttcaagtgggagcgcgtgatgaacttcgaggacggcggcgtggtgaccgtgacccaggactcctccctgcaggacggcgagttcatctac
aaggtgaagctgcgcggcaccaacttcccctccgacggccccgtaatgcagaagaagaccatgggctgggaggcctcctccgagcggatgtacccc
gaggacggcgccctgaagggcgagatcaagcagaggctgaagctgaaggacggcggccactacgacgctgaggtcaagaccacctacaaggcca
agaagcccgtgcagctgcccggcgcctacaacgtcaacatcaagttggacatcacctcccacaacgaggactacaccatcgtggaacagtacgaacg
cgccgagggccgccactccaccggcggcatggacgagctgTACaaggattacaaggatgacgatgacaagAATTCCGCGTAGCG
CTAGCTTCCTTGCCAGCGCCACGCGACTAGTGCCACAAACTTCTCTCTGCTAAAGCAAGC
AGGTGATGTTGAAGAAAACCCAGGGCCTGGAGGGTCCGAGGGCAGGGGAAGTCTCCTAACA
TGCGGGGACGTGGAGGAAAATCCCGGCCCATCCGGATATCCCTACGATGTGCCCGATTACGC
TCATatggtgagcaagggcgaggaggataacatggcctctctcccagcgacacatgagttacacatctttggctccatcaacggtgtggactttgaca
tggtgggtcagggcaccggcaatccaaatgatggttatgaggagttaaacctgaagtccaccaagggtgacctccagttctccccctggattctggtccct
catatcgggtatggcttccatcagtacctgccctaccctgacgggatgtcgcctttccaggccgccatggtagatggcAGCggataccaagtccatcgc
acaatgcagtttgaagatggtgcctcccttactgttaactaccgctacacctacgagggaagccacatcaaaggagaggcccaggtgaaggggactggt
ttccctgctgacggtcctgtgatgaccaactcgctgaccgctgcggactggtgcaggtcgaagaagacttaccccaacgacaaaaccatcatcagtacct
ttaagtggagttacaccactggaaatggcaagAGAtaccggagcactgcgcggaccacctacacctttgccaagccaatggcggctaactatctgaa
gaaccagccgatgtacgtgttccgtaagacggagctcaagcactccaagaccgagctcaacttcaaggagtggcaaaaggcctttaccgatgtgatgG
GAatggacGAGCTGTATaagGCTAGCTAAGCGGCCGCTCGAGTCTAGAGGGCCCGCGGTTCGAA
GGTAAGCCTATCCCTAACCCTCTCCTCGGTCTCGATTCTACGCGTACCGGTCATCATCACCAT
CACCATTGAGTTTAAACCCGCTGATCAGCCTCGACTGTGCCTTCTA
SEQ ID NO: 44 (see e.g., polypeptide in SEQ ID NO: 7) - mCherry-FLAG-UAG-UGG-
MS2-P2A-T2A-HA-mNeonGreen; UAG-UGG is bolded; MS2 is bold italicized
TAATACGACTCACTATAGGGAGACCCAAGCTGGCTAGGTAAGCTTGGTACCGAGCTCGGATC
Caccggtcgccaccatggtgagcaagggcgaggaggataacatggccatcatcaaggagttcatgcgcttcaaggtgcacatggagggctccgtgaa
cggccacgagttcgagatcgagggcgagggcgagggccgcccctacgagggcacccagaccgccaagctgaaggtgaccaagggtggccccctg
cccttcgcctgggacatcctgtcccctcagttcatgtacggctccaaggcctacgtgaagcaccccgccgacatccccgactacttgaagctgtccttccc
cgagggcttcaagtgggagcgcgtgatgaacttcgaggacggcggcgtggtgaccgtgacccaggactcctccctgcaggacggcgagttcatctac
aaggtgaagctgcgcggcaccaacttcccctccgacggccccgtaatgcagaagaagaccatgggctgggaggcctcctccgagcggatgtacccc
gaggacggcgccctgaagggcgagatcaagcagaggctgaagctgaaggacggcggccactacgacgctgaggtcaagaccacctacaaggcca
agaagcccgtgcagctgcccggcgcctacaacgtcaacatcaagttggacatcacctcccacaacgaggactacaccatcgtggaacagtacgaacg
cgccgagggccgccactccaccggcggcatggacgagctgTACaaggattacaaggatgacgatgacaagAATTCCGCGTAGCG
CTGGCTTTGCCAGCGCCACGCGaaACATGAGGATcACCCATGTACTAGTGCCACAAACTTCT
CTCTGCTAAAGCAAGCAGGTGATGTTGAAGAAAACCCAGGGCCTGGAGGGTCCGAGGGCAG
GGGAAGTCTCCTAACATGCGGGGACGTGGAGGAAAATCCCGGCCCATCCGGATATCCCTAC
GATGTGCCCGATTACGCTCATatggtgagcaagggcgaggaggataacatggcctctctcccagcgacacatgagttacacatcttt
ggctccatcaacggtgtggactttgacatggtgggtcagggcaccggcaatccaaatgatggttatgaggagttaaacctgaagtccaccaagggtgac
ctccagttctccccctggattctggtccctcatatcgggtatggcttccatcagtacctgccctaccctgacgggatgtcgcctttccaggccgccatggtag
atggcAGCggataccaagtccatcgcacaatgcagtttgaagatggtgcctcccttactgttaactaccgctacacctacgagggaagccacatcaaa
ggagaggcccaggtgaaggggactggtttccctgctgacggtcctgtgatgaccaactcgctgaccgctgcggactggtgcaggtcgaagaagactta
ccccaacgacaaaaccatcatcagtacctttaagtggagttacaccactggaaatggcaagAGAtaccggagcactgcgcggaccacctacaccttt
gccaagccaatggcggctaactatctgaagaaccagccgatgtacgtgttccgtaagacggagctcaagcactccaagaccgagctcaacttcaagga
gtggcaaaaggcctttaccgatgtgatgGGAatggacGAGCTGTATaagGCTAGCTAAGCGGCCGCTCGAGTCTAG
AGGGCCCGCGGTTCGAAGGTAAGCCTATCCCTAACCCTCTCCTCGGTCTCGATTCTACGCGT
ACCGGTCATCATCACCATCACCATTGAGTTTAAACCCGCTGATCAGCCTCGACTGTGCCTTCT
A
SEQ ID NO: 45 (see e.g., polypeptide in SEQ ID NO: 8) - mCherry-FLAG-UAG-UAG-
MS2-P2A-T2A-HA-mNeonGreen; UAG-UAG is bolded; MS2 is bold italicized
TAATACGACTCACTATAGGGAGACCCAAGCTGGCTAGGTAAGCTTGGTACCGAGCTCGGATC
Caccggtcgccaccatggtgagcaagggcgaggaggataacatggccatcatcaaggagttcatgcgcttcaaggtgcacatggagggctccgtgaa
cggccacgagttcgagatcgagggcgagggcgagggccgcccctacgagggcacccagaccgccaagctgaaggtgaccaagggtggccccctg
cccttcgcctgggacatcctgtcccctcagttcatgtacggctccaaggcctacgtgaagcaccccgccgacatccccgactacttgaagctgtccttccc
cgagggcttcaagtgggagcgcgtgatgaacttcgaggacggcggcgtggtgaccgtgacccaggactcctccctgcaggacggcgagttcatctac
aaggtgaagctgcgcggcaccaacttcccctccgacggccccgtaatgcagaagaagaccatgggctgggaggcctcctccgagcggatgtacccc
gaggacggcgccctgaagggcgagatcaagcagaggctgaagctgaaggacggcggccactacgacgctgaggtcaagaccacctacaaggcca
agaagcccgtgcagctgcccggcgcctacaacgtcaacatcaagttggacatcacctcccacaacgaggactacaccatcgtggaacagtacgaacg
cgccgagggccgccactccaccggcggcatggacgagctgTACaaggattacaaggatgacgatgacaagAATTCCGCGTAGCG
CTAGCTTTGCCAGCGCCACGCGaaACATGAGGATcACCCATGTACTAGTGCCACAAACTTCT
CTCTGCTAAAGCAAGCAGGTGATGTTGAAGAAAACCCAGGGCCTGGAGGGTCCGAGGGCAG
GGGAAGTCTCCTAACATGCGGGGACGTGGAGGAAAATCCCGGCCCATCCGGATATCCCTAC
GATGTGCCCGATTACGCTCATatggtgagcaagggcgaggaggataacatggcctctctcccagcgacacatgagttacacatcttt
ggctccatcaacggtgtggactttgacatggtgggtcagggcaccggcaatccaaatgatggttatgaggagttaaacctgaagtccaccaagggtgac
ctccagttctccccctggattctggtccctcatatcgggtatggcttccatcagtacctgccctaccctgacgggatgtcgcctttccaggccgccatggtag
atggcAGCggataccaagtccatcgcacaatgcagtttgaagatggtgcctcccttactgttaactaccgctacacctacgagggaagccacatcaaa
ggagaggcccaggtgaaggggactggtttccctgctgacggtcctgtgatgaccaactcgctgaccgctgcggactggtgcaggtcgaagaagactta
ccccaacgacaaaaccatcatcagtacctttaagtggagttacaccactggaaatggcaagAGAtaccggagcactgcgcggaccacctacaccttt
gccaagccaatggcggctaactatctgaagaaccagccgatgtacgtgttccgtaagacggagctcaagcactccaagaccgagctcaacttcaagga
gtggcaaaaggcctttaccgatgtgatgGGAatggacGAGCTGTATaagGCTAGCTAAGCGGCCGCTCGAGTCTAG
AGGGCCCGCGGTTCGAAGGTAAGCCTATCCCTAACCCTCTCCTCGGTCTCGATTCTACGCGT
ACCGGTCATCATCACCATCACCATTGAGTTTAAACCCGCTGATCAGCCTCGACTGTGCCTTCT
A
SEQ ID NO: 46 (see e.g., polypeptide in SEQ ID NO: 9) - mCherry-FLAG-P2A-T2A-UAG-
UAG-PP7-P2A-T2A-HA-mNeonGreen; UAG-UAG is bolded; PP7 is bold italicized.
TAATACGACTCACTATAGGGAGACCCAAGCTGGCTAGGTAAGCTTGGTACCGAGCTCGGATC
Caccggtcgccaccatggtgagcaagggcgaggaggataacatggccatcatcaaggagttcatgcgcttcaaggtgcacatggagggctccgtgaa
cggccacgagttcgagatcgagggcgagggcgagggccgcccctacgagggcacccagaccgccaagctgaaggtgaccaagggtggccccctg
cccttcgcctgggacatcctgtcccctcagttcatgtacggctccaaggcctacgtgaagcaccccgccgacatccccgactacttgaagctgtccttccc
cgagggcttcaagtgggagcgcgtgatgaacttcgaggacggcggcgtggtgaccgtgacccaggactcctccctgcaggacggcgagttcatctac
aaggtgaagctgcgcggcaccaacttcccctccgacggccccgtaatgcagaagaagaccatgggctgggaggcctcctccgagcggatgtacccc
gaggacggcgccctgaagggcgagatcaagcagaggctgaagctgaaggacggcggccactacgacgctgaggtcaagaccacctacaaggcca
agaagcccgtgcagctgcccggcgcctacaacgtcaacatcaagttggacatcacctcccacaacgaggactacaccatcgtggaacagtacgaacg
cgccgagggccgccactccaccggcggcatggacgagctgTACaaggattacaaggatgacgatgacaaaGGTAGCGGGGCAACT
AATTTTAGCTTACTCAAACAGGCTGGGGACGTCGAGGAGAATCCAGGCCCTGCATCCGCTGG
CTCTGGAGAAGGACGAGGCTCCTTGCTCACCTGTGGAGATGTCGAAGAGAACCCAGGTCCT
GCAACCGGGAATTCCGCGTAGCGCTAGCTTTGCCAGCGCCACGCGaaggagcagacgatatggcgtc
gctccaaTACTAGTGCCACAAACTTCTCTCTGCTAAAGCAAGCAGGTGATGTTGAAGAAAACCC
AGGGCCTGGAGGGTCCGAGGGCAGGGGAAGTCTCCTAACATGCGGGGACGTGGAGGAAAA
TCCCGGCCCATCCGGATATCCCTACGATGTGCCCGATTACGCTCATatggtgagcaagggcgaggaggata
acatggcctctctcccagcgacacatgagttacacatctttggctccatcaacggtgtggactttgacatggtgggtcagggcaccggcaatccaaatgat
ggttatgaggagttaaacctgaagtccaccaagggtgacctccagttctccccctggattctggtccctcatatcgggtatggcttccatcagtacctgccct
accctgacgggatgtcgcctttccaggccgccatggtagatggcAGCggataccaagtccatcgcacaatgcagtttgaagatggtgcctcccttactg
ttaactaccgctacacctacgagggaagccacatcaaaggagaggcccaggtgaaggggactggtttccctgctgacggtcctgtgatgaccaactcgc
tgaccgctgcggactggtgcaggtcgaagaagacttaccccaacgacaaaaccatcatcagtacctttaagtggagttacaccactggaaatggcaag
AGAtaccggagcactgcgcggaccacctacacctttgccaagccaatggggctaactatctgaagaaccagccgatgtacgtgttccgtaagacgg
agctcaagcactccaagaccgagctcaacttcaaggagtggcaaaaggcctttaccgatgtgatgGGAatggacGAGCTGTATaagGCT
AGCTAAGCGGCCGCTCGAGTCTAGAGGGCCCGCGGTTCGAAGGTAAGCCTATCCCTAACCCT
CTCCTCGGTCTCGATTCTACGCGTACCGGTCATCATCACCATCACCATTGAGTTTAAACCCGC
TGATCAGCCTCGACTGTGCCTTCTA
SEQ ID NO: 47 (see e.g., polypeptide in SEQ ID NO: 10) - mCherry-FLAG-P2A-T2A-
UAG-UAG-BoxB-P2A-T2A-HA-mNeonGreen; UAG-UAG is bolded; BoxB is bold italicized.
TAATACGACTCACTATAGGGAGACCCAAGCTGGCTAGGTAAGCTTGGTACCGAGCTCGGATC
Caccggtcgccaccatggtgagcaagggcgaggaggataacatggccatcatcaaggagttcatgcgcttcaaggtgcacatggagggctccgtgaa
cggccacgagttcgagatcgagggcgagggcgagggccgcccctacgagggcacccagaccgccaagctgaaggtgaccaagggtggccccctg
cccttcgcctgggacatcctgtcccctcagttcatgtacggctccaaggcctacgtgaagcaccccgccgacatccccgactacttgaagctgtccttccc
cgagggcttcaagtgggagcgcgtgatgaacttcgaggacggcggcgtggtgaccgtgacccaggactcctccctgcaggacggcgagttcatctac
aaggtgaagctgcgcggcaccaacttcccctccgacggccccgtaatgcagaagaagaccatgggctgggaggcctcctccgagcggatgtacccc
gaggacggcgccctgaagggcgagatcaagcagaggctgaagctgaaggacggcggccactacgacgctgaggtcaagaccacctacaaggcca
agaagcccgtgcagctgcccggcgcctacaacgtcaacatcaagttggacatcacctcccacaacgaggactacaccatcgtggaacagtacgaacg
cgccgagggccgccactccaccggcggcatggacgagctgTACaaggattacaaggatgacgatgacaaaGGTAGCGGGGCAACT
AATTTTAGCTTACTCAAACAGGCTGGGGACGTCGAGGAGAATCCAGGCCCTGCATCCGCTGG
CTCTGGAGAAGGACGAGGCTCCTTGCTCACCTGTGGAGATGTCGAAGAGAACCCAGGTCCT
GCAACCGGgAATTCCGCGTAGCGCTAGCTTTGCCAGCGCCACGCGgtaagggccctgaagaagggcc
caACTAGTGCCACAAACTTCTCTCTGCTAAAGCAAGCAGGTGATGTTGAAGAAAACCCAGGG
CCTGGAGGGTCCGAGGGCAGGGGAAGTCTCCTAACATGCGGGGACGTGGAGGAAAATCCCG
GCCCATCCGGATATCCCTACGATGTGCCCGATTACGCTCATatggtgagcaagggcgaggaggataacatggcc
tctctcccagcgacacatgagttacacatctttggctccatcaacggtgtggactttgacatggtgggtcagggcaccggcaatccaaatgatggttatgag
gagttaaacctgaagtccaccaagggtgacctccagttctccccctggattctggtccctcatatcgggtatggcttccatcagtacctgccctaccctgac
gggatgtcgcctttccaggccgccatggtagatggcAGCggataccaagtccatcgcacaatgcagtttgaagatggtgcctcccttactgttaactac
cgctacacctacgagggaagccacatcaaaggagaggcccaggtgaaggggactggtttccctgctgacggtcctgtgatgaccaactcgctgaccgc
tgcggactggtgcaggtcgaagaagacttaccccaacgacaaaaccatcatcagtacctttaagtggagttacaccactggaaatggcaagAGAtac
cggagcactgcgcggaccacctacacctttgccaagccaatggcggctaactatctgaagaaccagccgatgtacgtgttccgtaagacggagctcaa
gcactccaagaccgagctcaacttcaaggagtggcaaaaggcctttaccgatgtgatgGGAatggacGAGCTGTATaagGCTAGCTA
AGCGGCCGCTCGAGTCTAGAGGGCCCGCGGTTCGAAGGTAAGCCTATCCCTAACCCTCTCCT
CGGTCTCGATTCTACGCGTACCGGTCATCATCACCATCACCATTGAGTTTAAACCCGCTGATC
AGCCTCGACTGTGCCTTCTA
SEQ ID NO: 48 (see e.g., polypeptide in SEQ ID NO: 11) - mCherry-FLAG-P2A-T2A-
UAG-UAG-HIV TAR-P2A-T2A-HA-mNeonGreen; UAG-UAG is bolded; HIV TAR is bold italicized
TAATACGACTCACTATAGGGAGACCCAAGCTGGCTAGGTAAGCTTGGTACCGAGCTCGGATC
Caccggtcgccaccatggtgagcaagggcgaggaggataacatggccatcatcaaggagttcatgcgcttcaaggtgcacatggagggctccgtgaa
cggccacgagttcgagatcgagggcgagggcgagggccgcccctacgagggcacccagaccgccaagctgaaggtgaccaagggtggccccctg
cccttcgcctgggacatcctgtcccctcagttcatgtacggctccaaggcctacgtgaagcaccccgccgacatccccgactacttgaagctgtccttccc
cgagggcttcaagtgggagcgcgtgatgaacttcgaggacggcggcgtggtgaccgtgacccaggactcctccctgcaggacggcgagttcatctac
aaggtgaagctgcgcggcaccaacttcccctccgacggccccgtaatgcagaagaagaccatgggctgggaggcctcctccgagcggatgtacccc
gaggacggcgccctgaagggcgagatcaagcagaggctgaagctgaaggacggcggccactacgacgctgaggtcaagaccacctacaaggcca
agaagcccgtgcagctgcccggcgcctacaacgtcaacatcaagttggacatcacctcccacaacgaggactacaccatcgtggaacagtacgaacg
cgccgagggccgccactccaccggcggcatggacgagctgTACaaggattacaaggatgacgatgacaaaGGTAGCGGGGCAACT
AATTTTAGCTTACTCAAACAGGCTGGGGACGTCGAGGAGAATCCAGGCCCTGCATCCGCTGG
CTCTGGAGAAGGACGAGGCTCCTTGCTCACCTGTGGAGATGTCGAAGAGAACCCAGGTCCT
GCAACCGGgAATTCCGCGTAGCGCTAGCTTTGCCAGCGCCACGCGgtaggctcgtctgagctcattagct
ccgagccaACTAGTGCCACAAACTTCTCTCTGCTAAAGCAAGCAGGTGATGTTGAAGAAAACCC
AGGGCCTGGAGGGTCCGAGGGCAGGGGAAGTCTCCTAACATGCGGGGACGTGGAGGAAAA
TCCCGGCCCATCCGGATATCCCTACGATGTGCCCGATTACGCTCATatggtgagcaagggcgaggaggata
acatggcctctctcccagcgacacatgagttacacatctttggctccatcaacggtgtggactttgacatggtgggtcagggcaccggcaatccaaatgat
ggttatgaggagttaaacctgaagtccaccaagggtgacctccagttctccccctggattctggtccctcatatcgggtatggcttccatcagtacctgccct
accctgacgggatgtcgcctttccaggccgccatggtagatggcAGCggataccaagtccatcgcacaatgcagtttgaagatggtgcctcccttactg
ttaactaccgctacacctacgagggaagccacatcaaaggagaggcccaggtgaaggggactggtttccctgctgacggtcctgtgatgaccaactcgc
tgaccgctgcggactggtgcaggtcgaagaagacttaccccaacgacaaaaccatcatcagtacctttaagtggagttacaccactggaaatggcaag
AGAtaccggagcactgcgcggaccacctacacctttgccaagccaatggcggctaactatctgaagaaccagccgatgtacgtgttccgtaagacgg
agctcaagcactccaagaccgagctcaacttcaaggagtggcaaaaggcctttaccgatgtgatgGGAatggacGAGCTGTATaagGCT
AGCTAAGCGGCCGCTCGAGTCTAGAGGGCCCGCGGTTCGAAGGTAAGCCTATCCCTAACCCT
CTCCTCGGTCTCGATTCTACGCGTACCGGTCATCATCACCATCACCATTGAGTTTAAACCCGC
TGATCAGCCTCGACTGTGCCTTCTA
SEQ ID NO: 49 (see e.g., polypeptide in SEQ ID NO: 12) - PCP-linker-ADAR2(E488Q)
DD-TagBFP
TAATACGACTCACTATAGGGAGACCCAAGCTGGctagaggatcgaacccggtaccgccaccATGtccAAGACA
ATCGTGCTTAGCGTGGGAGAAGCAACTCGAACCCTCACAGAAATCCAATCAACAGCCGACC
GACAGATTTTTGAAGAGAAAGTGGGTCCCTTGGTTGGCCGGCTCAGGCTCACAGCCAGCTTG
AGACAGAACGGAGCCAAGACTGCCTATCGAGTGAATCTCAAGCTGGACCAGGCCGATGTAG
TGGACAGTGGTTTGCCCAAGGTTAGATACACGCAAGTGTGGTCACACGATGTCACTATTGTG
GCAAACTCCACCGAGGCAAGCAGGAAGTCCCTGTATGACCTGACCAAATCTTTGGTGGCGA
CGAGCCAAGTAGAGGATCTGGTGGTAAACCTGGTGCCCCTGGGAAGAGCCTCAACCGGTtctg
ggatttacgggggcagtgggagcggtgcaggatctggtagtccagctgggggaggagcaccgggtagcggtggggggtctcagctgcacctgcccc
aggttctcgcagacgccgtatcccgccttgtactgggcaagtttggtgatcttactgacaatttttcatctcctcatgcgaggcggaaagtactcgcaggcgt
cgtcatgacgaccggaactgacgtgaaagacgccaaagtcatctctgtctccacgggcacaaagtgcataaacggggagtacatgagcgaccggggg
ctggcactgaatgattgtcacgctgaaataatatctaggcgatctctgcttagatttctctacactcaactcgaattgtaccttaacaacaaagatgaccagaa
acgcagtatatttcagaaatcagaacgcggcggatttcgacttaaggaaaacgttcagttccacttgtatatcagcacatccccttgcggtgacgcccgaat
cttttccccgcacgagccgatattggaggagcccgcggacagacatcctaataggaaggctagaggccaacttcggacgaagattgaaagtggccagg
gtactatcccggtgcggtccaacgctagtattcaaacgtgggacggagtccttcaaggtgaacggctgttgacaatgagctgctcagacaaaatcgcgcg
ctggaatgtagtgggaatccaaggcagcctcttgagcatattcgtagaacccatatatttctcatccattattttgggctctctgtatcatggtgaccatctgtca
agggctatgtaccaacgaatttctaatatcgaggatcttcctccactctatacactcaataagcctctcttgtccgggatatcaaacgctgaggcccgccagc
cagggaaagctcctaacttcagtgttaactggaccgttggtgattctgcgatagaggtcatcaacgccacgacaggtaaggatgagctcggtagagcctc
acgcctgtgtaaacacgcgttgtattgtagatggatgagagtacatgggaaggtcccatctcacttgctccgaagcaagatcactaagcctaatgtgtatca
tgagtcaaaactcgcggctaaagaataccaggcagccaaagctcgactttttacagcttttattaaggcagggctcggggcatgggtcgagaagccgac
cgagcaggaccaattctctctgacggggagcggatccAGCGAGCTGATTAAGGAGAACATGCACATGAAGCTGTA
CATGGAGGGCACCGTGGACAACCATCACTTCAAGTGCACATCCGAGGGCGAAGGCAAGCCC
TACGAGGGCACCCAGACCATGAGAATCAAGGTGGTCGAGGGCGGCCCTCTCCCCTTCGCCTT
CGACATCCTGGCTACTAGCTTCCTCTACGGCAGCAAGACCTTCATCAACCACACCCAGGGCA
TCCCCGACTTCTTCAAGCAGTCCTTCCCTGAGGGCTTCACATGGGAGAGAGTCACCACATAC
GAAGACGGGGGCGTGCTGACCGCTACCCAGGACACCAGCCTCCAGGACGGCTGCCTCATCT
ACAACGTCAAGATCAGAGGGGTGAACTTCACATCCAACGGCCCTGTGATGCAGAAGAAAAC
ACTCGGCTGGGAGGCCTTCACCGAGACGCTGTACCCCGCTGACGGCGGCCTGGAAGGCAGA
AACGACATGGCCCTGAAGCTCGTGGGCGGGAGCCATCTGATCGCAAACATCAAGACCACAT
ATAGATCCAAGAAACCCGCTAAGAACCTCAAGATGCCTGGCGTCTACTATGTGGACTACAG
ACTGGAAAGAATCAAGGAGGCCAACAACGAGACCTACGTCGAGCAGCACGAGGTGGCAGT
GGCCAGATACTGCGACCTCCCTAGCAAACTGGGGCACAAGCTTAATtAAGGGCCCGTTTAAA
CCCGCTGATCAGCCTCGACTGTGCCTTCTA
SEQ ID NO: 50 (see e.g., polypeptide in SEQ ID NO: 13) - λN-linker-ADAR2(E488Q) DD-
TagBFP
TAATACGACTCACTATAGGGAGACCCAAGCTGGctagaggatcgaacccggtaccgccaccATGGCCgacgcac
aaacacgacgacgtgagcgtcgcgctgagaaacaagctcaatggaaagctgcaaacACCGGTtctgggatttacgggggcagtgggagcggtg
caggatctggtagtccagctgggggaggagcaccgggtagcggtggggggtctcagctgcacctgccccaggttctcgcagacgccgtatcccgcctt
gtactgggcaagtttggtgatcttactgacaatttttcatctcctcatgcgaggcggaaagtactcgcaggcgtcgtcatgacgaccggaactgacgtgaaa
gacgccaaagtcatctctgtctccacgggcacaaagtgcataaacggggagtacatgagcgaccgggggctggcactgaatgattgtcacgctgaaat
aatatctaggcgatctctgcttagatttctctacactcaactcgaattgtaccttaacaacaaagatgaccagaaacgcagtatatttcagaaatcagaacgc
ggcggatttcgacttaaggaaaacgttcagttccacttgtatatcagcacatccccttgcggtgacgcccgaatcttttccccgcacgagccgatattggag
gagcccgcggacagacatcctaataggaaggctagaggccaacttcggacgaagattgaaagtggccagggtactatcccggtgcggtccaacgcta
gtattcaaacgtgggacggagtccttcaaggtgaacggctgttgacaatgagctgctcagacaaaatcgcgcgctggaatgtagtgggaatccaaggca
gcctcttgagcatattcgtagaacccatatatttctcatccattattttgggctctctgtatcatggtgaccatctgtcaagggctatgtaccaacgaatttctaat
atcgaggatcttcctccactctatacactcaataagcctctcttgtccgggatatcaaacgctgaggcccgccagccagggaaagctcctaacttcagtgtt
aactggaccgttggtgattctgcgatagaggtcatcaacgccacgacaggtaaggatgagctcggtagagcctcacgcctgtgtaaacacgcgttgtatt
gtagatggatgagagtacatgggaaggtcccatctcacttgctccgaagcaagatcactaagcctaatgtgtatcatgagtcaaaactcgcggctaaaga
ataccaggcagccaaagctcgactttttacagcttttattaaggcagggctcggggcatgggtcgagaagccgaccgagcaggaccaattctctctgacg
gggagcggatccAGCGAGCTGATTAAGGAGAACATGCACATGAAGCTGTACATGGAGGGCACCGT
GGACAACCATCACTTCAAGTGCACATCCGAGGGCGAAGGCAAGCCCTACGAGGGCACCCAG
ACCATGAGAATCAAGGTGGTCGAGGGCGGCCCTCTCCCCTTCGCCTTCGACATCCTGGCTAC
TAGCTTCCTCTACGGCAGCAAGACCTTCATCAACCACACCCAGGGCATCCCCGACTTCTTCA
AGCAGTCCTTCCCTGAGGGCTTCACATGGGAGAGAGTCACCACATACGAAGACGGGGGCGT
GCTGACCGCTACCCAGGACACCAGCCTCCAGGACGGCTGCCTCATCTACAACGTCAAGATCA
GAGGGGTGAACTTCACATCCAACGGCCCTGTGATGCAGAAGAAAACACTCGGCTGGGAGGC
CTTCACCGAGACGCTGTACCCCGCTGACGGCGGCCTGGAAGGCAGAAACGACATGGCCCTG
AAGCTCGTGGGCGGGAGCCATCTGATCGCAAACATCAAGACCACATATAGATCCAAGAAAC
CCGCTAAGAACCTCAAGATGCCTGGCGTCTACTATGTGGACTACAGACTGGAAAGAATCAA
GGAGGCCAACAACGAGACCTACGTCGAGCAGCACGAGGTGGCAGTGGCCAGATACTGCGAC
CTCCCTAGCAAACTGGGGCACAAGCTTAATtAAGGGCCCGTTTAAACCCGCTGATCAGCCTC
GACTGTGCCTTCTA
SEQ ID NO: 51 (see e.g., polypeptide in SEQ ID NO: 14) - HIV-tat-linker-ADAR2(E488Q)
DD-TagBFP
TAATACGACTCACTATAGGGAGACCCAAGCTGGctagaggatcgaacccggtaccgccaccATGGCCtctggtcc
tcgtccccgtggtactcgtggtaaaggtcgccgtattcgtcgcACCGGTtctgggatttacgggggcagtgggagcggtgcaggatctggtagtcc
agctgggggaggagcaccgggtagcggtggggggtctcagctgcacctgccccaggttctcgcagacgccgtatcccgccttgtactgggcaagtttg
gtgatcttactgacaatttttcatctcctcatgcgaggcggaaagtactcgcaggcgtcgtcatgacgaccggaactgacgtgaaagacgccaaagtcatc
tctgtctccacgggcacaaagtgcataaacggggagtacatgagcgaccgggggctggcactgaatgattgtcacgctgaaataatatctaggcgatctc
tgcttagatttctctacactcaactcgaattgtaccttaacaacaaagatgaccagaaacgcagtatatttcagaaatcagaacgcggcggatttcgacttaa
ggaaaacgttcagttccacttgtatatcagcacatccccttgcggtgacgcccgaatcttttccccgcacgagccgatattggaggagcccgcggacaga
catcctaataggaaggctagaggccaacttcggacgaagattgaaagtggccagggtactatcccggtgcggtccaacgctagtattcaaacgtgggac
ggagtccttcaaggtgaacggctgttgacaatgagctgctcagacaaaatcgcgcgctggaatgtagtgggaatccaaggcagcctcttgagcatattcg
tagaacccatatatttctcatccattattttgggctctctgtatcatggtgaccatctgtcaagggctatgtaccaacgaatttctaatatcgaggatcttcctcca
ctctatacactcaataagcctctcttgtccgggatatcaaacgctgaggcccgccagccagggaaagctcctaacttcagtgttaactggaccgttggtgat
tctgcgatagaggtcatcaacgccacgacaggtaaggatgagctcggtagagcctcacgcctgtgtaaacacgcgttgtattgtagatggatgagagtac
atgggaaggtcccatctcacttgctccgaagcaagatcactaagcctaatgtgtatcatgagtcaaaactcgcggctaaagaataccaggcagccaaagc
tcgactttttacagcttttattaaggcagggctcggggcatgggtcgagaagccgaccgagcaggaccaattctctctgacggggagcggatccAGC
GAGCTGATTAAGGAGAACATGCACATGAAGCTGTACATGGAGGGCACCGTGGACAACCATC
ACTTCAAGTGCACATCCGAGGGCGAAGGCAAGCCCTACGAGGGCACCCAGACCATGAGAAT
CAAGGTGGTCGAGGGCGGCCCTCTCCCCTTCGCCTTCGACATCCTGGCTACTAGCTTCCTCTA
CGGCAGCAAGACCTTCATCAACCACACCCAGGGCATCCCCGACTTCTTCAAGCAGTCCTTCC
CTGAGGGCTTCACATGGGAGAGAGTCACCACATACGAAGACGGGGGCGTGCTGACCGCTAC
CCAGGACACCAGCCTCCAGGACGGCTGCCTCATCTACAACGTCAAGATCAGAGGGGTGAAC
TTCACATCCAACGGCCCTGTGATGCAGAAGAAAACACTCGGCTGGGAGGCCTTCACCGAGA
CGCTGTACCCCGCTGACGGCGGCCTGGAAGGCAGAAACGACATGGCCCTGAAGCTCGTGGG
CGGGAGCCATCTGATCGCAAACATCAAGACCACATATAGATCCAAGAAACCCGCTAAGAAC
CTCAAGATGCCTGGCGTCTACTATGTGGACTACAGACTGGAAAGAATCAAGGAGGCCAACA
ACGAGACCTACGTCGAGCAGCACGAGGTGGCAGTGGCCAGATACTGCGACCTCCCTAGCAA
ACTGGGGCACAAGCTTAATAAGGGCCCGTTTAAACCCGCTGATCAGCCTCGACTGTGCCTTC
TA
SEQ ID NO: 52 (see e.g., polypeptide in SEQ ID NO: 15 & 16) - dTomato-BiCMV-
EGFPd2-UAG-UAG-MS2-polyA; UAG-UAG is bolded; MS2 is bold italicized; polyA is bold double-
underlined.
cggcagtgaaaaaaatgctttatttgtgaaatttgtgatgctattgctttatttgtaaccattataagctgcaataaacaagttaacaacaacaattgcattcatttt
atgtttcaggttcagggggaggtgtgggaggttttttaaagcaagtaaaacctctacaaatgtggtatggctgattatgatcctctagactgcagcctcagga
gatctgggcccctacttgtacagctcgtccatgccgtacaggaacaggtggtggcggccctcggagcgctcgtactgttccacgatggtgtagtcctcgtt
gtgggaggtgatgtccagcttggtgtccacgtagtagtagccgggcagttgcacgggcttcttggccatgtagatggtcttgaactccaccaggtagtggc
cgccgtccttcagcttcagggcctggtggatctcgcccttcagcacgccgtcgcgggggtacaggcgctcggtggaggcctcccagcccatggtcttctt
ctgcattacggggccgtcgggggggaagttggtgccgcgcatcttcaccttgtagatcagcgtgccgtcctgcagggaggagtcctgggtcacggtcac
cagaccgccgtcctcgaagttcatcacgcgctcccacttgaagccctcggggaaggacagcttcttgtaatcggggatgtcggcggggtgcttcacgta
cgccttggagccgtacatgaactggggggacaggatgtcccaggcgaagggcagggggccgcccttggtcaccttcagcttggcggtctgggtgccc
tcgtaggggcggccctcgccctcgccctcgatctcgaactcgtggccgttcatggagccctccatgcgcaccttgaagcgcatgaactctttgatgacctc
ctcgcccttgctcaccatggtggcgaattctccaggcgatctgacggttcactaaacgagctctgcttatataggcctcccaccgtacacgccacctcgac
atactcgagtagttattaatagtaatcaattacggggtcattagttcatagcccatatatggagttccgcgttacataacttacggtaaatggcccgcctggct
gaccgcccaacgacccccgcccattgacgtcaataatgacgtatgttcccatagtaacgccaatagggactttccattgacgtcaatgggtggagtatttac
ggtaaactgcccacttggcagtacatcaagtgtatcatatgccaagtacgccccctattgacgtcaatgacggtaaatggcccgcctggcattatgcccag
tacatgaccttatgggactttcctacttggcagtacatctacgtattagtcatcgctattaccatggtgatgcggttttggcagtacatcaatgggcgtggatag
cggtttgactcacggggatttccaagtctccaccccattgacgtcaatgggagtttgttttggcaccaaaatcaacgggactttccaaaatgtcgtaacaact
ccgccccattgacgcaaatgggcggtaggcgtgtacggtgggaggtctatataagcagagctggtttagtgaaccgtcagatccgctagggatcctcta
gtcagctgacgcgtgctagcgatatcggcgcgccagcatttaaatctgtacagaccggtcgccaccatggtgagcaagggcgaggagctgttcaccgg
ggtggtgcccatcctggtcgagctggacggcgacgtaaacggccacaagttcagcgtgtccggcgagggcgagggcgatgccacctacggcaagct
gaccctgaagttcatctgcaccaccggcaagctgcccgtgccctggcccaccctcgtgaccaccctgacctacggcgtgcagtgcttcagccgctaccc
cgaccacatgaagcagcacgacttcttcaagtccgccatgcccgaaggctacgtccaggagcgcaccatcttcttcaaggacgacggcaactacaaga
cccgcgccgaggtgaagttcgagggcgacaccctggtgaaccgcatcgagctgaagggcatcgacttcaaggaggacggcaacatcctggggcaca
agctggagtacaactacaacagccacaacgtctatatcatggccgacaagcagaagaacggcatcaaggtgaacttcaagatccgccacaacatcgag
gacggcagcgtgcagctcgccgaccactaccagcagaacacccccatcggcgacggccccgtgctgctgcccgacaaccactacctgagcacccag
tccgccctgagcaaagaccccaacgagaagcgcgatcacatggtcctgctggagttcgtgaccgccgccgggatcactctcggcatggacgagctgta
caagaagcttagccatggcttcccgccggaggtggaggagcaggatgatggcacgctgcccatgtcttgtgcccaggagagcgggatggaccgtcac
cctgcagcctgtgcttctgcCCggAtcAACgtgTAGCGCTAGCTTTGCCAGCGCCACACGaaACATGAGGA
TcACCCATGTcggccgcactcctcaggtgcaggctgcctatcagaaggtggtggctggtgtggccaatgccctggctcacaaataccactgaga
tctttttccctctgccaaaaattatggggacatcatgaagccccttgGgcatctgacttctggctaataaaggaaatttattttcattgcaatagtgtgttgg
aattttttgtgtctctcactcggaaggacatatgggagggcaaatcatttaaaacatcagaatgagt
SEQ ID NO: 53 (see e.g., polypeptide in SEQ ID NO: 17) - MCP-linker-ADAR2-DDN-
Bad(F)- ADAR2(E488Q)-DDC-TagBFP
TAATACGACTCACTATAGGGAGACCCAAGCTGGctagaggatcgaacccttaaggccaccatggcgtccaatttcactca
gtttgtgctggttgacaacggcgggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCGGGTCGGGCACCGGTGCTC
CACCCAATCTCTGGGCAGCGCAGCGCTACGGCCGTGAGCTCAGAAGGATGTCCGATGAGttcG
TCGACagacatcctaataggaaggctagaggccaacttcggacgaagattgaaagtggccagggtactatcccggtgcggtccaacgctagtattc
aaacgtgggacggagtccttcaaggtgaacggctgttgacaatgagctgctcagacaaaatcgcgcgctggaatgtagtgggaatccaaggcagcctct
tgagcatattcgtagaacccatatatttctcatccattattttgggctctctgtatcatggtgaccatctgtcaagggctatgtaccaacgaatttctaatatcgag
gatcttcctccactctatacactcaataagcctctcttgtccgggatatcaaacgctgaggcccgccagccagggaaagctcctaacttcagtgttaactgg
accgttggtgattctgcgatagaggtcatcaacgccacgacaggtaaggatgagctcggtagagcctcacgcctgtgtaaacacgcgttgtattgtagatg
gatgagagtacatgggaaggtcccatctcacttgctccgaagcaagatcactaagcctaatgtgtatcatgagtcaaaactcgcggctaaagaataccag
gcagccaaagctcgactttttacagcttttattaaggcagggctcggggcatgggtcgagaagccgaccgagcaggaccaattctctctgacggggagc
ggatccAGCGAGCTGATTAAGGAGAACATGCACATGAAGCTGTACATGGAGGGCACCGTGGAC
AACCATCACTTCAAGTGCACATCCGAGGGCGAAGGCAAGCCCTACGAGGGCACCCAGACCA
TGAGAATCAAGGTGGTCGAGGGCGGCCCTCTCCCCTTCGCCTTCGACATCCTGGCTACTAGC
TTCCTCTACGGCAGCAAGACCTTCATCAACCACACCCAGGGCATCCCCGACTTCTTCAAGCA
GTCCTTCCCTGAGGGCTTCACATGGGAGAGAGTCACCACATACGAAGACGGGGGCGTGCTG
ACCGCTACCCAGGACACCAGCCTCCAGGACGGCTGCCTCATCTACAACGTCAAGATCAGAG
GGGTGAACTTCACATCCAACGGCCCTGTGATGCAGAAGAAAACACTCGGCTGGGAGGCCTT
CACCGAGACGCTGTACCCCGCTGACGGCGGCCTGGAAGGCAGAAACGACATGGCCCTGAAG
CTCGTGGGCGGGAGCCATCTGATCGCAAACATCAAGACCACATATAGATCCAAGAAACCCG
CTAAGAACCTCAAGATGCCTGGCGTCTACTATGTGGACTACAGACTGGAAAGAATCAAGGA
GGCCAACAACGAGACCTACGTCGAGCAGCACGAGGTGGCAGTGGCCAGATACTGCGACCTC
CCTAGCAAACTGGGGCACAAGCTTAATtAAGGGCCCGTTTAAACCCGCTGATCAGCCTCGAC
TGTGCCTTCTA
SEQ ID NO: 54 (see e.g., polypeptide in SEQ ID NO: 18) - MCP-linker-ADAR2-DDN-
Bad(F)- ADAR2(E488Q)-DDC-Bcl-xL-TagBFP
TAATACGACTCACTATAGGGAGACCCAAGCTGGctagaggatcgaacccttaaggccaccatggcgtccaatttcactca
gtttgtgctggttgacaacggcgggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCGGGTCGGGCACCGGTGCTC
CACCCAATCTCTGGGCAGCGCAGCGCTACGGCCGTGAGCTCAGAAGGATGTCCGATGAGttcG
TCGACagacatcctaataggaaggctagaggccaacttcggacgaagattgaaagtggccagggtactatcccggtgcggtccaacgctagtattc
aaacgtgggacggagtccttcaaggtgaacggctgttgacaatgagctgctcagacaaaatcgcgcgctggaatgtagtgggaatccaaggcagcctct
tgagcatattcgtagaacccatatatttctcatccattattttgggctctctgtatcatggtgaccatctgtcaagggctatgtaccaacgaatttctaatatcgag
gatcttcctccactctatacactcaataagcctctcttgtccgggatatcaaacgctgaggcccgccagccagggaaagctcctaacttcagtgttaactgg
accgttggtgattctgcgatagaggtcatcaacgccacgacaggtaaggatgagctcggtagagcctcacgcctgtgtaaacacgcgttgtattgtagatg
gatgagagtacatgggaaggtcccatctcacttgctccgaagcaagatcactaagcctaatgtgtatcatgagtcaaaactcgcggctaaagaataccag
gcagccaaagctcgactttttacagcttttattaaggcagggctcggggcatgggtcgagaagccgaccgagcaggaccaattctctctgacggggagc
GCGGCCGGAGGTAGCGGCGGAAGCGCGGCCGCTTCAAGTAACCGGGAGCTGGTGGTTGACT
TTCTCTCCTACAAGCTTTCCCAGAAAGGATACAGCTGGAGTCAGTTTAGTGATGTGGAAGAG
AACAGGACTGAGGCCCCAGAAGGGACTGAATCGGAGATGGAGACCCCCAGTGCCATCAATG
GCAACCCATCCTGGCACCTGGCAGACAGCCCCGCGGTGAATGGAGCCACTGGCCACAGCAG
CAGTTTGGATGCCCGGGAGGTGATCCCCATGGCAGCAGTAAAGCAAGCGCTGAGGGAGGCA
GGCGACGAGTTTGAACTGCGGTACCGGCGGGCATTCAGTGACCTGACATCCCAGCTCCACAT
CACCCCAGGGACAGCATATCAGAGCTTTGAACAGGTAGTGAATGAACTCTTCCGGGATGGG
GTAAACTGGGGTCGCATTGTGGCCTTTTTCTCCTTCGGCGGGGCACTGTGCGTGGAAAGCGT
AGACAAGGAGATGCAGGTATTGGTGAGTCGGATCGCAGCTTGGATGGCCACTTACCTGAAT
GACCACCTAGAGCCTTGGATCCAGGAGAACGGCGGCTGGGATACTTTTGTGGAACTCTATGG
GAACAATggatccAGCGAGCTGATTAAGGAGAACATGCACATGAAGCTGTACATGGAGGGCAC
CGTGGACAACCATCACTTCAAGTGCACATCCGAGGGCGAAGGCAAGCCCTACGAGGGCACC
CAGACCATGAGAATCAAGGTGGTCGAGGGCGGCCCTCTCCCCTTCGCCTTCGACATCCTGGC
TACTAGCTTCCTCTACGGCAGCAAGACCTTCATCAACCACACCCAGGGCATCCCCGACTTCT
TCAAGCAGTCCTTCCCTGAGGGCTTCACATGGGAGAGAGTCACCACATACGAAGACGGGGG
CGTGCTGACCGCTACCCAGGACACCAGCCTCCAGGACGGCTGCCTCATCTACAACGTCAAGA
TCAGAGGGGTGAACTTCACATCCAACGGCCCTGTGATGCAGAAGAAAACACTCGGCTGGGA
GGCCTTCACCGAGACGCTGTACCCCGCTGACGGCGGCCTGGAAGGCAGAAACGACATGGCC
CTGAAGCTCGTGGGCGGGAGCCATCTGATCGCAAACATCAAGACCACATATAGATCCAAGA
AACCCGCTAAGAACCTCAAGATGCCTGGCGTCTACTATGTGGACTACAGACTGGAAAGAAT
CAAGGAGGCCAACAACGAGACCTACGTCGAGCAGCACGAGGTGGCAGTGGCCAGATACTGC
GACCTCCCTAGCAAACTGGGGCACAAGCTTAATAAGGGCCCGTTTAAACCCGCTGATCAGC
CTCGACTGTGCCTTCTA
SEQ ID NO: 55 (see e.g., polypeptide in SEQ ID NO: 19) - MCP-linker-ADAR2-DDN-Bim-
ADAR2(E488Q)-DDC-TagBFP
TAATACGACTCACTATAGGGAGACCCAAGCTGGctagaggatcgaacccttaaggccaccatggcgtccaatttcactca
gtttgtgctggttgacaacggcgggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCGGGTCGGGCTCTGGAGAC
ATGCGGCCAGAGATTTGGATCGCACAGGAACTGAGGCGCATTGGCGATGAGTTCAATGCAT
ACTATGCCCGAAGAACCGGTGACagacatcctaataggaaggctagaggccaacttcggacgaagattgaaagtggccagggta
ctatcccggtgcggtccaacgctagtattcaaacgtgggacggagtccttcaaggtgaacggctgttgacaatgagctgctcagacaaaatcgcgcgctg
gaatgtagtgggaatccaaggcagcctcttgagcatattcgtagaacccatatatttctcatccattattttgggctctctgtatcatggtgaccatctgtcaag
ggctatgtaccaacgaatttctaatatcgaggatcttcctccactctatacactcaataagcctctcttgtccgggatatcaaacgctgaggcccgccagcca
gggaaagctcctaacttcagtgttaactggaccgttggtgattctgcgatagaggtcatcaacgccacgacaggtaaggatgagctcggtagagcctcac
gcctgtgtaaacacgcgttgtattgtagatggatgagagtacatgggaaggtcccatctcacttgctccgaagcaagatcactaagcctaatgtgtatcatg
agtcaaaactcgcggctaaagaataccaggcagccaaagctcgactttttacagcttttattaaggcagggctcggggcatgggtcgagaagccgaccg
agcaggaccaattctctctgacggggagcggatccAGCGAGCTGATTAAGGAGAACATGCACATGAAGCTGTAC
ATGGAGGGCACCGTGGACAACCATCACTTCAAGTGCACATCCGAGGGCGAAGGCAAGCCCT
ACGAGGGCACCCAGACCATGAGAATCAAGGTGGTCGAGGGCGGCCCTCTCCCCTTCGCCTTC
GACATCCTGGCTACTAGCTTCCTCTACGGCAGCAAGACCTTCATCAACCACACCCAGGGCAT
CCCCGACTTCTTCAAGCAGTCCTTCCCTGAGGGCTTCACATGGGAGAGAGTCACCACATACG
AAGACGGGGGCGTGCTGACCGCTACCCAGGACACCAGCCTCCAGGACGGCTGCCTCATCTA
CAACGTCAAGATCAGAGGGGTGAACTTCACATCCAACGGCCCTGTGATGCAGAAGAAAACA
CTCGGCTGGGAGGCCTTCACCGAGACGCTGTACCCCGCTGACGGCGGCCTGGAAGGCAGAA
ACGACATGGCCCTGAAGCTCGTGGGCGGGAGCCATCTGATCGCAAACATCAAGACCACATA
TAGATCCAAGAAACCCGCTAAGAACCTCAAGATGCCTGGCGTCTACTATGTGGACTACAGAC
TGGAAAGAATCAAGGAGGCCAACAACGAGACCTACGTCGAGCAGCACGAGGTGGCAGTGG
CCAGATACTGCGACCTCCCTAGCAAACTGGGGCACAAGCTTAATtAAGGGCCCGTTTAAACC
CGCTGATCAGCCTCGACTGTGCCTTCTA
SEQ ID NO: 56 (see e.g., polypeptide in SEQ ID NO: 20) - MCP-linker-ADAR2-DDN-Bim-
ADAR2(E488Q)-DDC-Bcl-xL-TagBFP
TAATACGACTCACTATAGGGAGACCCAAGCTGGctagaggatcgaacccttaaggccaccatggcgtccaatttcactca
gtttgtgctggttgacaacggcgggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCGGGTCGGGCTCTGGAGAC
ATGCGGCCAGAGATTTGGATCGCACAGGAACTGAGGCGCATTGGCGATGAGTTCAATGCAT
ACTATGCCCGAAGAACCGGTGACagacatcctaataggaaggctagaggccaacttcggacgaagattgaaagtggccagggta
ctatcccggtgcggtccaacgctagtattcaaacgtgggacggagtccttcaaggtgaacggctgttgacaatgagctgctcagacaaaatcgcgcgctg
gaatgtagtgggaatccaaggcagcctcttgagcatattcgtagaacccatatatttctcatccattattttgggctctctgtatcatggtgaccatctgtcaag
ggctatgtaccaacgaatttctaatatcgaggatcttcctccactctatacactcaataagcctctcttgtccgggatatcaaacgctgaggcccgccagcca
gggaaagctcctaacttcagtgttaactggaccgttggtgattctgcgatagaggtcatcaacgccacgacaggtaaggatgagctcggtagagcctcac
gcctgtgtaaacacgcgttgtattgtagatggatgagagtacatgggaaggtcccatctcacttgctccgaagcaagatcactaagcctaatgtgtatcatg
agtcaaaactcgcggctaaagaataccaggcagccaaagctcgactttttacagcttttattaaggcagggctcggggcatgggtcgagaagccgaccg
agcaggaccaattctctctgacggggagcGCGGCCGGAGGTAGCGGCGGAAGCGCGGCCGCTTCAAGTAACC
GGGAGCTGGTGGTTGACTTTCTCTCCTACAAGCTTTCCCAGAAAGGATACAGCTGGAGTCAG
TTTAGTGATGTGGAAGAGAACAGGACTGAGGCCCCAGAAGGGACTGAATCGGAGATGGAGA
CCCCCAGTGCCATCAATGGCAACCCATCCTGGCACCTGGCAGACAGCCCCGCGGTGAATGG
AGCCACTGGCCACAGCAGCAGTTTGGATGCCCGGGAGGTGATCCCCATGGCAGCAGTAAAG
CAAGCGCTGAGGGAGGCAGGCGACGAGTTTGAACTGCGGTACCGGCGGGCATTCAGTGACC
TGACATCCCAGCTCCACATCACCCCAGGGACAGCATATCAGAGCTTTGAACAGGTAGTGAAT
GAACTCTTCCGGGATGGGGTAAACTGGGGTCGCATTGTGGCCTTTTTCTCCTTCGGCGGGGC
ACTGTGCGTGGAAAGCGTAGACAAGGAGATGCAGGTATTGGTGAGTCGGATCGCAGCTTGG
ATGGCCACTTACCTGAATGACCACCTAGAGCCTTGGATCCAGGAGAACGGCGGCTGGGATA
CTTTTGTGGAACTCTATGGGAACAATggatccAGCGAGCTGATTAAGGAGAACATGCACATGAA
GCTGTACATGGAGGGCACCGTGGACAACCATCACTTCAAGTGCACATCCGAGGGCGAAGGC
AAGCCCTACGAGGGCACCCAGACCATGAGAATCAAGGTGGTCGAGGGCGGCCCTCTCCCCT
TCGCCTTCGACATCCTGGCTACTAGCTTCCTCTACGGCAGCAAGACCTTCATCAACCACACCC
AGGGCATCCCCGACTTCTTCAAGCAGTCCTTCCCTGAGGGCTTCACATGGGAGAGAGTCACC
ACATACGAAGACGGGGGCGTGCTGACCGCTACCCAGGACACCAGCCTCCAGGACGGCTGCC
TCATCTACAACGTCAAGATCAGAGGGGTGAACTTCACATCCAACGGCCCTGTGATGCAGAA
GAAAACACTCGGCTGGGAGGCCTTCACCGAGACGCTGTACCCCGCTGACGGCGGCCTGGAA
GGCAGAAACGACATGGCCCTGAAGCTCGTGGGCGGGAGCCATCTGATCGCAAACATCAAGA
CCACATATAGATCCAAGAAACCCGCTAAGAACCTCAAGATGCCTGGCGTCTACTATGTGGAC
TACAGACTGGAAAGAATCAAGGAGGCCAACAACGAGACCTACGTCGAGCAGCACGAGGTG
GCAGTGGCCAGATACTGCGACCTCCCTAGCAAACTGGGGCACAAGCTTAATtAAGGGCCCGT
TTAAACCCGCTGATCAGCCTCGACTGTGCCTTCTA
SEQ ID NO: 57 (see e.g., polypeptide in SEQ ID NO: 21) - MCP-linker-ADAR2-DDN-
MS1(A)- ADAR2(E488Q)-DDC-TagBFP
TAATACGACTCACTATAGGGAGACCCAAGCTGGctagaggatcgaacccttaaggccaccatggcgtccaatttcactca
gtttgtgctggttgacaacggcgggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCGGAGGTAGCGGCGGATCT
GGGCGACCAGAAATCTGGATGACACAAGGTTTACGCAGACTCGGAGATGAGGCAAATGCTT
ACTATGCTAGACGGACCGGTGACagacatcctaataggaaggctagaggccaacttcggacgaagattgaaagtggccagggta
ctatcccggtgcggtccaacgctagtattcaaacgtgggacggagtccttcaaggtgaacggctgttgacaatgagctgctcagacaaaatcgcgcgctg
gaatgtagtgggaatccaaggcagcctcttgagcatattcgtagaacccatatatttctcatccattattttgggctctctgtatcatggtgaccatctgtcaag
ggctatgtaccaacgaatttctaatatcgaggatcttcctccactctatacactcaataagcctctcttgtccgggatatcaaacgctgaggcccgccagcca
gggaaagctcctaacttcagtgttaactggaccgttggtgattctgcgatagaggtcatcaacgccacgacaggtaaggatgagctcggtagagcctcac
gcctgtgtaaacacgcgttgtattgtagatggatgagagtacatgggaaggtcccatctcacttgctccgaagcaagatcactaagcctaatgtgtatcatg
agtcaaaactcgcggctaaagaataccaggcagccaaagctcgactttttacagcttttattaaggcagggctcggggcatgggtcgagaagccgaccg
agcaggaccaattctctctgacggggagcggatccAGCGAGCTGATTAAGGAGAACATGCACATGAAGCTGTAC
ATGGAGGGCACCGTGGACAACCATCACTTCAAGTGCACATCCGAGGGCGAAGGCAAGCCCT
ACGAGGGCACCCAGACCATGAGAATCAAGGTGGTCGAGGGCGGCCCTCTCCCCTTCGCCTTC
GACATCCTGGCTACTAGCTTCCTCTACGGCAGCAAGACCTTCATCAACCACACCCAGGGCAT
CCCCGACTTCTTCAAGCAGTCCTTCCCTGAGGGCTTCACATGGGAGAGAGTCACCACATACG
AAGACGGGGGCGTGCTGACCGCTACCCAGGACACCAGCCTCCAGGACGGCTGCCTCATCTA
CAACGTCAAGATCAGAGGGGTGAACTTCACATCCAACGGCCCTGTGATGCAGAAGAAAACA
CTCGGCTGGGAGGCCTTCACCGAGACGCTGTACCCCGCTGACGGCGGCCTGGAAGGCAGAA
ACGACATGGCCCTGAAGCTCGTGGGCGGGAGCCATCTGATCGCAAACATCAAGACCACATA
TAGATCCAAGAAACCCGCTAAGAACCTCAAGATGCCTGGCGTCTACTATGTGGACTACAGAC
TGGAAAGAATCAAGGAGGCCAACAACGAGACCTACGTCGAGCAGCACGAGGTGGCAGTGG
CCAGATACTGCGACCTCCCTAGCAAACTGGGGCACAAGCTTAATtAAGGGCCCGTTTAAACC
CGCTGATCAGCCTCGACTGTGCCTTCTA
SEQ ID NO: 58 (see e.g., polypeptide in SEQ ID NO: 22) - MCP-linker-ADAR2-DDN-
MS1(A)- ADAR2(E488Q)-DDC-Mcl-1-TagBFP
TAATACGACTCACTATAGGGAGACCCAAGCTGGctagaggatcgaacccttaaggccaccatggcgtccaatttcactca
gtttgtgctggttgacaacggcgggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCGGAGGTAGCGGCGGATCT
GGGCGACCAGAAATCTGGATGACACAAGGTTTACGCAGACTCGGAGATGAGGCAAATGCTT
ACTATGCTAGACGGACCGGTGACagacatcctaataggaaggctagaggccaacttcggacgaagattgaaagtggccagggta
ctatcccggtgcggtccaacgctagtattcaaacgtgggacggagtccttcaaggtgaacggctgttgacaatgagctgctcagacaaaatcgcgcgctg
gaatgtagtgggaatccaaggcagcctcttgagcatattcgtagaacccatatatttctcatccattattttgggctctctgtatcatggtgaccatctgtcaag
ggctatgtaccaacgaatttctaatatcgaggatcttcctccactctatacactcaataagcctctcttgtccgggatatcaaacgctgaggcccgccagcca
gggaaagctcctaacttcagtgttaactggaccgttggtgattctgcgatagaggtcatcaacgccacgacaggtaaggatgagctcggtagagcctcac
gcctgtgtaaacacgcgttgtattgtagatggatgagagtacatgggaaggtcccatctcacttgctccgaagcaagatcactaagcctaatgtgtatcatg
agtcaaaactcgcggctaaagaataccaggcagccaaagctcgactttttacagcttttattaaggcagggctcggggcatgggtcgagaagccgaccg
agcaggaccaattctctctgacggggagcGGTACCGgCGgTccaggggacgagttgtaccggcagtcgctggagattatctctCGCtacctt
cgggagcaggccaccGGAgccaaggacacaaagccaatgggcaggtctggggccaccagcaggaaggcgctgGAAaccttacgacgggttg
gggatggcgtgcagcgcaaccacgagACTgccttccaaggcatgcttcggaaactggacatcaaaaacgaaGATgatgtgaaatcgttgtctAG
GgtgatgatccatgttttcagcgacggcgtaacaaactggggcaggattgtgactctcatttcttttggtgcctttgtggctaaacacttgAAAaccataa
accaagaaagctgcatcgaaccattagcagaaagtatcacagacgttctcgtaaggacaaaacgggactggctagttaaacaaagaggctgggatggg
tttgtggagttcttccatgtagaggacctagaaggtggcggatccAGCGAGCTGATTAAGGAGAACATGCACATGAAGC
TGTACATGGAGGGCACCGTGGACAACCATCACTTCAAGTGCACATCCGAGGGCGAAGGCAA
GCCCTACGAGGGCACCCAGACCATGAGAATCAAGGTGGTCGAGGGCGGCCCTCTCCCCTTC
GCCTTCGACATCCTGGCTACTAGCTTCCTCTACGGCAGCAAGACCTTCATCAACCACACCCA
GGGCATCCCCGACTTCTTCAAGCAGTCCTTCCCTGAGGGCTTCACATGGGAGAGAGTCACCA
CATACGAAGACGGGGGCGTGCTGACCGCTACCCAGGACACCAGCCTCCAGGACGGCTGCCT
CATCTACAACGTCAAGATCAGAGGGGTGAACTTCACATCCAACGGCCCTGTGATGCAGAAG
AAAACACTCGGCTGGGAGGCCTTCACCGAGACGCTGTACCCCGCTGACGGCGGCCTGGAAG
GCAGAAACGACATGGCCCTGAAGCTCGTGGGCGGGAGCCATCTGATCGCAAACATCAAGAC
CACATATAGATCCAAGAAACCCGCTAAGAACCTCAAGATGCCTGGCGTCTACTATGTGGACT
ACAGACTGGAAAGAATCAAGGAGGCCAACAACGAGACCTACGTCGAGCAGCACGAGGTGG
CAGTGGCCAGATACTGCGACCTCCCTAGCAAACTGGGGCACAAGCTTAATtAAGGGCCCGTT
TAAACCCGCTGATCAGCCTCGACTGTGCCTTCTA
SEQ ID NO: 59 (see e.g., polypeptide in SEQ ID NO: 23) - MCP-linker-ADAR2-DDN-
Bad(L)- ADAR2(E488Q)-DDC-Bcl-xL-TagBFP
TAATACGACTCACTATAGGGAGACCCAAGCTGGctagaggatcgaacccttaaggccaccatggcgtccaatttcactca
gtttgtgctggttgacaacggcgggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCGGGTCGGGCACCGGTGCTC
CACCCAATCTCTGGGCAGCGCAGCGCTACGGCCGTGAGCTCAGAAGGATGTCCGATGAGcTG
GTCGACagacatcctaataggaaggctagaggccaacttcggacgaagattgaaagtggccagggtactatcccggtgcggtccaacgctagtatt
caaacgtgggacggagtccttcaaggtgaacggctgttgacaatgagctgctcagacaaaatcgcgcgctggaatgtagtgggaatccaaggcagcct
cttgagcatattcgtagaacccatatatttctcatccattattttgggctctctgtatcatggtgaccatctgtcaagggctatgtaccaacgaatttctaatatcg
aggatcttcctccactctatacactcaataagcctctcttgtccgggatatcaaacgctgaggcccgccagccagggaaagctcctaacttcagtgttaact
ggaccgttggtgattctgcgatagaggtcatcaacgccacgacaggtaaggatgagctcggtagagcctcacgcctgtgtaaacacgcgttgtattgtag
atggatgagagtacatgggaaggtcccatctcacttgctccgaagcaagatcactaagcctaatgtgtatcatgagtcaaaactcgcggctaaagaatacc
aggcagccaaagctcgactttttacagcttttattaaggcagggctcggggcatgggtcgagaagccgaccgagcaggaccaattctctctgacgggga
gcGCGGCCGGAGGTAGCGGCGGAAGCGCGGCCGCTTCAAGTAACCGGGAGCTGGTGGTTGA
CTTTCTCTCCTACAAGCTTTCCCAGAAAGGATACAGCTGGAGTCAGTTTAGTGATGTGGAAG
AGAACAGGACTGAGGCCCCAGAAGGGACTGAATCGGAGATGGAGACCCCCAGTGCCATCAA
TGGCAACCCATCCTGGCACCTGGCAGACAGCCCCGCGGTGAATGGAGCCACTGGCCACAGC
AGCAGTTTGGATGCCCGGGAGGTGATCCCCATGGCAGCAGTAAAGCAAGCGCTGAGGGAGG
CAGGCGACGAGTTTGAACTGCGGTACCGGCGGGCATTCAGTGACCTGACATCCCAGCTCCAC
ATCACCCCAGGGACAGCATATCAGAGCTTTGAACAGGTAGTGAATGAACTCTTCCGGGATG
GGGTAAACTGGGGTCGCATTGTGGCCTTTTTCTCCTTCGGCGGGGCACTGTGCGTGGAAAGC
GTAGACAAGGAGATGCAGGTATTGGTGAGTCGGATCGCAGCTTGGATGGCCACTTACCTGA
ATGACCACCTAGAGCCTTGGATCCAGGAGAACGGCGGCTGGGATACTTTTGTGGAACTCTAT
GGGAACAATggatccAGCGAGCTGATTAAGGAGAACATGCACATGAAGCTGTACATGGAGGGC
ACCGTGGACAACCATCACTTCAAGTGCACATCCGAGGGCGAAGGCAAGCCCTACGAGGGCA
CCCAGACCATGAGAATCAAGGTGGTCGAGGGCGGCCCTCTCCCCTTCGCCTTCGACATCCTG
GCTACTAGCTTCCTCTACGGCAGCAAGACCTTCATCAACCACACCCAGGGCATCCCCGACTT
CTTCAAGCAGTCCTTCCCTGAGGGCTTCACATGGGAGAGAGTCACCACATACGAAGACGGG
GGCGTGCTGACCGCTACCCAGGACACCAGCCTCCAGGACGGCTGCCTCATCTACAACGTCAA
GATCAGAGGGGTGAACTTCACATCCAACGGCCCTGTGATGCAGAAGAAAACACTCGGCTGG
GAGGCCTTCACCGAGACGCTGTACCCCGCTGACGGCGGCCTGGAAGGCAGAAACGACATGG
CCCTGAAGCTCGTGGGCGGGAGCCATCTGATCGCAAACATCAAGACCACATATAGATCCAA
GAAACCCGCTAAGAACCTCAAGATGCCTGGCGTCTACTATGTGGACTACAGACTGGAAAGA
ATCAAGGAGGCCAACAACGAGACCTACGTCGAGCAGCACGAGGTGGCAGTGGCCAGATACT
GCGACCTCCCTAGCAAACTGGGGCACAAGCTTAATtAAGGGCCCGTTTAAACCCGCTGATCA
GCCTCGACTGTGCCTTCTA
SEQ ID NO: 60 (see e.g., polypeptide in SEQ ID NO: 24) - MCP-linker-ADAR2-DDN-
MS1(I)- ADAR2(E488Q)-DDC-Mcl-1-TagBFP
TAATACGACTCACTATAGGGAGACCCAAGCTGGctagaggatcgaacccttaaggccaccatggcgtccaatttcactca
gtttgtgctggttgacaacggcgggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCGGAGGTAGCGGCGGATCT
GGGCGACCAGAAATCTGGATGACACAAGGTTTACGCAGACTCGGAGATGAGATAAATGCTT
ACTATGCTAGACGGACCGGTGACagacatcctaataggaaggctagaggccaacttcggacgaagattgaaagtggccagggta
ctatcccggtgcggtccaacgctagtattcaaacgtgggacggagtccttcaaggtgaacggctgttgacaatgagctgctcagacaaaatcgcgcgctg
gaatgtagtgggaatccaaggcagcctcttgagcatattcgtagaacccatatatttctcatccattattttgggctctctgtatcatggtgaccatctgtcaag
ggctatgtaccaacgaatttctaatatcgaggatcttcctccactctatacactcaataagcctctcttgtccgggatatcaaacgctgaggcccgccagcca
gggaaagctcctaacttcagtgttaactggaccgttggtgattctgcgatagaggtcatcaacgccacgacaggtaaggatgagctcggtagagcctcac
gcctgtgtaaacacgcgttgtattgtagatggatgagagtacatgggaaggtcccatctcacttgctccgaagcaagatcactaagcctaatgtgtatcatg
agtcaaaactcgcggctaaagaataccaggcagccaaagctcgactttttacagcttttattaaggcagggctcggggcatgggtcgagaagccgaccg
agcaggaccaattctctctgacggggagcGGTACCGgCGgTccaggggacgagttgtaccggcagtcgctggagattatctctCGCtacctt
cgggagcaggccaccGGAgccaaggacacaaagccaatgggcaggtctggggccaccagcaggaaggcgctgGAAaccttacgacgggttg
gggatggcgtgcagcgcaaccacgagACTgccttccaaggcatgcttcggaaactggacatcaaaaacgaaGATgatgtgaaatcgttgtctAG
GgtgatgatccatgttttcagcgacggcgtaacaaactggggcaggattgtgactctcatttcttttggtgcctttgtggctaaacacttgAAAaccataa
accaagaaagctgcatcgaaccattagcagaaagtatcacagacgttctcgtaaggacaaaacgggactggctagttaaacaaagaggctgggatggg
tttgtggagttcttccatgtagaggacctagaaggtggcggatccAGCGAGCTGATTAAGGAGAACATGCACATGAAGC
TGTACATGGAGGGCACCGTGGACAACCATCACTTCAAGTGCACATCCGAGGGCGAAGGCAA
GCCCTACGAGGGCACCCAGACCATGAGAATCAAGGTGGTCGAGGGCGGCCCTCTCCCCTTC
GCCTTCGACATCCTGGCTACTAGCTTCCTCTACGGCAGCAAGACCTTCATCAACCACACCCA
GGGCATCCCCGACTTCTTCAAGCAGTCCTTCCCTGAGGGCTTCACATGGGAGAGAGTCACCA
CATACGAAGACGGGGGCGTGCTGACCGCTACCCAGGACACCAGCCTCCAGGACGGCTGCCT
CATCTACAACGTCAAGATCAGAGGGGTGAACTTCACATCCAACGGCCCTGTGATGCAGAAG
AAAACACTCGGCTGGGAGGCCTTCACCGAGACGCTGTACCCCGCTGACGGCGGCCTGGAAG
GCAGAAACGACATGGCCCTGAAGCTCGTGGGCGGGAGCCATCTGATCGCAAACATCAAGAC
CACATATAGATCCAAGAAACCCGCTAAGAACCTCAAGATGCCTGGCGTCTACTATGTGGACT
ACAGACTGGAAAGAATCAAGGAGGCCAACAACGAGACCTACGTCGAGCAGCACGAGGTGG
CAGTGGCCAGATACTGCGACCTCCCTAGCAAACTGGGGCACAAGCTTAATtAAGGGCCCGTT
TAAACCCGCTGATCAGCCTCGACTGTGCCTTCTA
SEQ ID NO: 61 (see e.g., polypeptide in SEQ ID NO: 25) - MCP-linker-ADAR2-DDN-
ALFA-ADAR2(E488Q)-DDC-TagBFP
TAATACGACTCACTATAGGGAGACCCAAGCTGGctagaggatcgaacccttaaggccaccatggcgtccaatttcactca
gtttgtgctggttgacaacggcgggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCCCATCCCGCCTGGAGGAAG
AACTTCGGAGGAGACTTACTGAGCCTACCGGTGACagacatcctaataggaaggctagaggccaacttcggacgaag
attgaaagtggccagggtactatcccggtgcggtccaacgctagtattcaaacgtgggacggagtccttcaaggtgaacggctgttgacaatgagctgct
cagacaaaatcgcgcgctggaatgtagtgggaatccaaggcagcctcttgagcatattcgtagaacccatatatttctcatccattattttgggctctctgtat
catggtgaccatctgtcaagggctatgtaccaacgaatttctaatatcgaggatcttcctccactctatacactcaataagcctctcttgtccgggatatcaaa
cgctgaggcccgccagccagggaaagctcctaacttcagtgttaactggaccgttggtgattctgcgatagaggtcatcaacgccacgacaggtaagga
tgagctcggtagagcctcacgcctgtgtaaacacgcgttgtattgtagatggatgagagtacatgggaaggtcccatctcacttgctccgaagcaagatca
ctaagcctaatgtgtatcatgagtcaaaactcgcggctaaagaataccaggcagccaaagctcgactttttacagcttttattaaggcagggctcggggcat
gggtcgagaagccgaccgagcaggaccaattctctctgacggggagcggatccAGCGAGCTGATTAAGGAGAACATGCAC
ATGAAGCTGTACATGGAGGGCACCGTGGACAACCATCACTTCAAGTGCACATCCGAGGGCG
AAGGCAAGCCCTACGAGGGCACCCAGACCATGAGAATCAAGGTGGTCGAGGGCGGCCCTCT
CCCCTTCGCCTTCGACATCCTGGCTACTAGCTTCCTCTACGGCAGCAAGACCTTCATCAACCA
CACCCAGGGCATCCCCGACTTCTTCAAGCAGTCCTTCCCTGAGGGCTTCACATGGGAGAGAG
TCACCACATACGAAGACGGGGGCGTGCTGACCGCTACCCAGGACACCAGCCTCCAGGACGG
CTGCCTCATCTACAACGTCAAGATCAGAGGGGTGAACTTCACATCCAACGGCCCTGTGATGC
AGAAGAAAACACTCGGCTGGGAGGCCTTCACCGAGACGCTGTACCCCGCTGACGGCGGCCT
GGAAGGCAGAAACGACATGGCCCTGAAGCTCGTGGGCGGGAGCCATCTGATCGCAAACATC
AAGACCACATATAGATCCAAGAAACCCGCTAAGAACCTCAAGATGCCTGGCGTCTACTATGT
GGACTACAGACTGGAAAGAATCAAGGAGGCCAACAACGAGACCTACGTCGAGCAGCACGA
GGTGGCAGTGGCCAGATACTGCGACCTCCCTAGCAAACTGGGGCACAAGCTTAATtAAGGGC
CCGTTTAAACCCGCTGATCAGCCTCGACTGTGCCTTCTA
SEQ ID NO: 62 (see e.g., polypeptide in SEQ ID NO: 26) - MCP-linker-ADAR2-DDN-
ALFA-ADAR2(E488Q)-DDC-NbALFA-TagBFP
TAATACGACTCACTATAGGGAGACCCAAGCTGGctagaggatcgaacccttaaggccaccatggcgtccaatttcactca
gtttgtgctggttgacaacggcgggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCCCATCCCGCCTGGAGGAAG
AACTTCGGAGGAGACTTACTGAGCCTACCGGTGACagacatcctaataggaaggctagaggccaacttcggacgaag
attgaaagtggccagggtactatcccggtgcggtccaacgctagtattcaaacgtgggacggagtccttcaaggtgaacggctgttgacaatgagctgct
cagacaaaatcgcgcgctggaatgtagtgggaatccaaggcagcctcttgagcatattcgtagaacccatatatttctcatccattattttgggctctctgtat
catggtgaccatctgtcaagggctatgtaccaacgaatttctaatatcgaggatcttcctccactctatacactcaataagcctctcttgtccgggatatcaaa
cgctgaggcccgccagccagggaaagctcctaacttcagtgttaactggaccgttggtgattctgcgatagaggtcatcaacgccacgacaggtaagga
tgagctcggtagagcctcacgcctgtgtaaacacgcgttgtattgtagatggatgagagtacatgggaaggtcccatctcacttgctccgaagcaagatca
ctaagcctaatgtgtatcatgagtcaaaactcgcggctaaagaataccaggcagccaaagctcgactttttacagcttttattaaggcagggctcggggcat
gggtcgagaagccgaccgagcaggaccaattctctctgacggggagcGGAGGTACCGCCGAAGTTCAATTACAGGAAT
CGGGTGGAGGTCTGGTACAACCTGGGGGCTCTCTTCGCCTGAGTTGCACTGCCAGTGGAGTT
ACGATTTCTGCACTTAATGCTATGGCGATGGGTTGGTATCGTCAGGCCCCAGGGGAACGTCG
CGTCATGGTCGCTGCCGTTTCCGAACGTGGCAATGCTATGTACCGCGAGTCTGTTCAGGGCC
GCTTCACGGTTACCCGCGATTTTACAAATAAAATGGTATCGTTGCAAATGGACAACTTAAAG
CCAGAGGACACTGCTGTGTACTACTGTCACGTCCTTGAAGATCGTGTGGATTCCTTTCATGAT
TATTGGGGGCAGGGGACTCAGGTCACTGTATCCTCAGGaGCTggatccAGCGAGCTGATTAAGG
AGAACATGCACATGAAGCTGTACATGGAGGGCACCGTGGACAACCATCACTTCAAGTGCAC
ATCCGAGGGCGAAGGCAAGCCCTACGAGGGCACCCAGACCATGAGAATCAAGGTGGTCGAG
GGCGGCCCTCTCCCCTTCGCCTTCGACATCCTGGCTACTAGCTTCCTCTACGGCAGCAAGACC
TTCATCAACCACACCCAGGGCATCCCCGACTTCTTCAAGCAGTCCTTCCCTGAGGGCTTCAC
ATGGGAGAGAGTCACCACATACGAAGACGGGGGCGTGCTGACCGCTACCCAGGACACCAGC
CTCCAGGACGGCTGCCTCATCTACAACGTCAAGATCAGAGGGGTGAACTTCACATCCAACGG
CCCTGTGATGCAGAAGAAAACACTCGGCTGGGAGGCCTTCACCGAGACGCTGTACCCCGCT
GACGGCGGCCTGGAAGGCAGAAACGACATGGCCCTGAAGCTCGTGGGCGGGAGCCATCTGA
TCGCAAACATCAAGACCACATATAGATCCAAGAAACCCGCTAAGAACCTCAAGATGCCTGG
CGTCTACTATGTGGACTACAGACTGGAAAGAATCAAGGAGGCCAACAACGAGACCTACGTC
GAGCAGCACGAGGTGGCAGTGGCCAGATACTGCGACCTCCCTAGCAAACTGGGGCACAAGC
TTAATtAAGGGCCCGTTTAAACCCGCTGATCAGCCTCGACTGTGCCTTCTA
SEQ ID NO: 63 (see e.g., polypeptide in SEQ ID NO: 27) - MCP-linker-ADAR2-DDN-
ALFA-PE- ADAR2(E488Q)-DDC-TagBFP
TAATACGACTCACTATAGGGAGACCCAAGCTGGctagaggatcgaacccttaaggccaccatggcgtccaatttcactca
gtttgtgctggttgacaacgggggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCGGATCAGGTCCAGGACGC
CTGGAGGAAGAACTTCGGAGGAGACTTTCTCCTGGAACCGGTGACagacatcctaataggaaggctagagg
ccaacttcggacgaagattgaaagtggccagggtactatcccggtgcggtccaacgctagtattcaaacgtgggacggagtccttcaaggtgaacggct
gttgacaatgagctgctcagacaaaatcgcgcgctggaatgtagtgggaatccaaggcagcctcttgagcatattcgtagaacccatatatttctcatccatt
attttgggctctctgtatcatggtgaccatctgtcaagggctatgtaccaacgaatttctaatatcgaggatcttcctccactctatacactcaataagcctctctt
gtccgggatatcaaacgctgaggcccgccagccagggaaagctcctaacttcagtgttaactggaccgttggtgattctgcgatagaggtcatcaacgcc
acgacaggtaaggatgagctcggtagagcctcacgcctgtgtaaacacgcgttgtattgtagatggatgagagtacatgggaaggtcccatctcacttgct
ccgaagcaagatcactaagcctaatgtgtatcatgagtcaaaactcgcggctaaagaataccaggcagccaaagctcgactttttacagcttttattaaggc
agggctcggggcatgggtcgagaagccgaccgagcaggaccaattctctctgacggggagcggatccAGCGAGCTGATTAAGGAG
AACATGCACATGAAGCTGTACATGGAGGGCACCGTGGACAACCATCACTTCAAGTGCACAT
CCGAGGGCGAAGGCAAGCCCTACGAGGGCACCCAGACCATGAGAATCAAGGTGGTCGAGG
GCGGCCCTCTCCCCTTCGCCTTCGACATCCTGGCTACTAGCTTCCTCTACGGCAGCAAGACCT
TCATCAACCACACCCAGGGCATCCCCGACTTCTTCAAGCAGTCCTTCCCTGAGGGCTTCACA
TGGGAGAGAGTCACCACATACGAAGACGGGGGCGTGCTGACCGCTACCCAGGACACCAGCC
TCCAGGACGGCTGCCTCATCTACAACGTCAAGATCAGAGGGGTGAACTTCACATCCAACGGC
CCTGTGATGCAGAAGAAAACACTCGGCTGGGAGGCCTTCACCGAGACGCTGTACCCCGCTG
ACGGCGGCCTGGAAGGCAGAAACGACATGGCCCTGAAGCTCGTGGGCGGGAGCCATCTGAT
CGCAAACATCAAGACCACATATAGATCCAAGAAACCCGCTAAGAACCTCAAGATGCCTGGC
GTCTACTATGTGGACTACAGACTGGAAAGAATCAAGGAGGCCAACAACGAGACCTACGTCG
AGCAGCACGAGGTGGCAGTGGCCAGATACTGCGACCTCCCTAGCAAACTGGGGCACAAGCT
TAATtAAGGGCCCGTTTAAACCCGCTGATCAGCCTCGACTGTGCCTTCTA
SEQ ID NO: 64 (see e.g., polypeptide in SEQ ID NO: 28) - MCP-linker-ADAR2-DDN-
ALFA-PE- ADAR2(E488Q)-DDC-NbALFA-TagBFP
TAATACGACTCACTATAGGGAGACCCAAGCTGGctagaggatcgaacccttaaggccaccatggcgtccaatttcactca
gtttgtgctggttgacaacggcgggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCGGATCAGGTCCAGGACGC
CTGGAGGAAGAACTTCGGAGGAGACTTTCTCCTGGAACCGGTGACagacatcctaataggaaggctagagg
ccaacttcggacgaagattgaaagtggccagggtactatcccggtgcggtccaacgctagtattcaaacgtgggacggagtccttcaaggtgaacggct
gttgacaatgagctgctcagacaaaatcgcgcgctggaatgtagtgggaatccaaggcagcctcttgagcatattcgtagaacccatatatttctcatccatt
attttgggctctctgtatcatggtgaccatctgtcaagggctatgtaccaacgaatttctaatatcgaggatcttcctccactctatacactcaataagcctctctt
gtccgggatatcaaacgctgaggcccgccagccagggaaagctcctaacttcagtgttaactggaccgttggtgattctgcgatagaggtcatcaacgcc
acgacaggtaaggatgagctcggtagagcctcacgcctgtgtaaacacgcgttgtattgtagatggatgagagtacatgggaaggtcccatctcacttgct
ccgaagcaagatcactaagcctaatgtgtatcatgagtcaaaactcgcggctaaagaataccaggcagccaaagctcgactttttacagcttttattaaggc
agggctcggggcatgggtcgagaagccgaccgagcaggaccaattctctctgacggggagcGGAGGTACCGCCGAAGTTCAAT
TACAGGAATCGGGTGGAGGTCTGGTACAACCTGGGGGCTCTCTTCGCCTGAGTTGCACTGCC
AGTGGAGTTACGATTTCTGCACTTAATGCTATGGCGATGGGTTGGTATCGTCAGGCCCCAGG
GGAACGTCGCGTCATGGTCGCTGCCGTTTCCGAACGTGGCAATGCTATGTACCGCGAGTCTG
TTCAGGGCCGCTTCACGGTTACCCGCGATTTTACAAATAAAATGGTATCGTTGCAAATGGAC
AACTTAAAGCCAGAGGACACTGCTGTGTACTACTGTCACGTCCTTGAAGATCGTGTGGATTC
CTTTCATGATTATTGGGGGCAGGGGACTCAGGTCACTGTATCCTCAGGaGCTggatccAGCGAG
CTGATTAAGGAGAACATGCACATGAAGCTGTACATGGAGGGCACCGTGGACAACCATCACT
TCAAGTGCACATCCGAGGGCGAAGGCAAGCCCTACGAGGGCACCCAGACCATGAGAATCAA
GGTGGTCGAGGGCGGCCCTCTCCCCTTCGCCTTCGACATCCTGGCTACTAGCTTCCTCTACGG
CAGCAAGACCTTCATCAACCACACCCAGGGCATCCCCGACTTCTTCAAGCAGTCCTTCCCTG
AGGGCTTCACATGGGAGAGAGTCACCACATACGAAGACGGGGGCGTGCTGACCGCTACCCA
GGACACCAGCCTCCAGGACGGCTGCCTCATCTACAACGTCAAGATCAGAGGGGTGAACTTC
ACATCCAACGGCCCTGTGATGCAGAAGAAAACACTCGGCTGGGAGGCCTTCACCGAGACGC
TGTACCCCGCTGACGGCGGCCTGGAAGGCAGAAACGACATGGCCCTGAAGCTCGTGGGCGG
GAGCCATCTGATCGCAAACATCAAGACCACATATAGATCCAAGAAACCCGCTAAGAACCTC
AAGATGCCTGGCGTCTACTATGTGGACTACAGACTGGAAAGAATCAAGGAGGCCAACAACG
AGACCTACGTCGAGCAGCACGAGGTGGCAGTGGCCAGATACTGCGACCTCCCTAGCAAACT
GGGGCACAAGCTTAATtAAGGGCCCGTTTAAACCCGCTGATCAGCCTCGACTGTGCCTTCTA
SEQ ID NO: 65 (see e.g., polypeptide in SEQ ID NO: 29) - MCP-linker-ADAR2-DDN-
ALFA-78- ADAR2(E488Q)-DDC-TagBFP
TAATACGACTCACTATAGGGAGACCCAAGCTGGctagaggatcgaacccttaaggccaccatggcgtccaatttcactca
gtttgtgctggttgacaacggcgggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCGGATCAGGTCCAGGCCGCC
TGGAGCAGGAAATTCGGGCAAGACTTTCTCCTGGAACCGGTGACagacatcctaataggaaggctagaggcc
aacttcggacgaagattgaaagtggccagggtactatcccggtgcggtccaacgctagtattcaaacgtgggacggagtccttcaaggtgaacggctgtt
gacaatgagctgctcagacaaaatcgcgcgctggaatgtagtgggaatccaaggcagcctcttgagcatattcgtagaacccatatatttctcatccattatt
ttgggctctctgtatcatggtgaccatctgtcaagggctatgtaccaacgaatttctaatatcgaggatcttcctccactctatacactcaataagcctctcttgt
ccgggatatcaaacgctgaggcccgccagccagggaaagctcctaacttcagtgttaactggaccgttggtgattctgcgatagaggtcatcaacgcca
cgacaggtaaggatgagctcggtagagcctcacgcctgtgtaaacacgcgttgtattgtagatggatgagagtacatgggaaggtcccatctcacttgctc
cgaagcaagatcactaagcctaatgtgtatcatgagtcaaaactcgcggctaaagaataccaggcagccaaagctcgactttttacagcttttattaaggca
gggctcggggcatgggtcgagaagccgaccgagcaggaccaattctctctgacggggagcggatccAGCGAGCTGATTAAGGAGA
ACATGCACATGAAGCTGTACATGGAGGGCACCGTGGACAACCATCACTTCAAGTGCACATC
CGAGGGCGAAGGCAAGCCCTACGAGGGCACCCAGACCATGAGAATCAAGGTGGTCGAGGG
CGGCCCTCTCCCCTTCGCCTTCGACATCCTGGCTACTAGCTTCCTCTACGGCAGCAAGACCTT
CATCAACCACACCCAGGGCATCCCCGACTTCTTCAAGCAGTCCTTCCCTGAGGGCTTCACAT
GGGAGAGAGTCACCACATACGAAGACGGGGGCGTGCTGACCGCTACCCAGGACACCAGCCT
CCAGGACGGCTGCCTCATCTACAACGTCAAGATCAGAGGGGTGAACTTCACATCCAACGGC
CCTGTGATGCAGAAGAAAACACTCGGCTGGGAGGCCTTCACCGAGACGCTGTACCCCGCTG
ACGGCGGCCTGGAAGGCAGAAACGACATGGCCCTGAAGCTCGTGGGCGGGAGCCATCTGAT
CGCAAACATCAAGACCACATATAGATCCAAGAAACCCGCTAAGAACCTCAAGATGCCTGGC
GTCTACTATGTGGACTACAGACTGGAAAGAATCAAGGAGGCCAACAACGAGACCTACGTCG
AGCAGCACGAGGTGGCAGTGGCCAGATACTGCGACCTCCCTAGCAAACTGGGGCACAAGCT
TAATtAAGGGCCCGTTTAAACCCGCTGATCAGCCTCGACTGTGCCTTCTA
SEQ ID NO: 66 (see e.g., polypeptide in SEQ ID NO: 30) - MCP-linker-ADAR2-DDN-
ALFA-PE- ADAR2(E488Q)-DDC-NbALFA-TagBFP
TAATACGACTCACTATAGGGAGACCCAAGCTGGctagaggatcgaacccttaaggccaccatggcgtccaatttcactca
gtttgtgctggttgacaacgggggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCGGATCAGGTCCAGGCCGCC
TGGAGCAGGAAATTCGGGCAAGACTTTCTCCTGGAACCGGTGACagacatcctaataggaaggctagaggcc
aacttcggacgaagattgaaagtggccagggtactatcccggtgcggtccaacgctagtattcaaacgtgggacggagtccttcaaggtgaacggctgtt
gacaatgagctgctcagacaaaatcgcgcgctggaatgtagtgggaatccaaggcagcctcttgagcatattcgtagaacccatatatttctcatccattatt
ttgggctctctgtatcatggtgaccatctgtcaagggctatgtaccaacgaatttctaatatcgaggatcttcctccactctatacactcaataagcctctcttgt
ccgggatatcaaacgctgaggcccgccagccagggaaagctcctaacttcagtgttaactggaccgttggtgattctgcgatagaggtcatcaacgcca
cgacaggtaaggatgagctcggtagagcctcacgcctgtgtaaacacgcgttgtattgtagatggatgagagtacatgggaaggtcccatctcacttgctc
cgaagcaagatcactaagcctaatgtgtatcatgagtcaaaactcgcggctaaagaataccaggcagccaaagctcgactttttacagcttttattaaggca
gggctcggggcatgggtcgagaagccgaccgagcaggaccaattctctctgacggggagcGGAGGTACCGCCGAAGTTCAATT
ACAGGAATCGGGTGGAGGTCTGGTACAACCTGGGGGCTCTCTTCGCCTGAGTTGCACTGCCA
GTGGAGTTACGATTTCTGCACTTAATGCTATGGCGATGGGTTGGTATCGTCAGGCCCCAGGG
GAACGTCGCGTCATGGTCGCTGCCGTTTCCGAACGTGGCAATGCTATGTACCGCGAGTCTGT
TCAGGGCCGCTTCACGGTTACCCGCGATTTTACAAATAAAATGGTATCGTTGCAAATGGACA
ACTTAAAGCCAGAGGACACTGCTGTGTACTACTGTCACGTCCTTGAAGATCGTGTGGATTCC
TTTCATGATTATTGGGGGCAGGGGACTCAGGTCACTGTATCCTCAGGaGCTggatccAGCGAGCT
GATTAAGGAGAACATGCACATGAAGCTGTACATGGAGGGCACCGTGGACAACCATCACTTC
AAGTGCACATCCGAGGGCGAAGGCAAGCCCTACGAGGGCACCCAGACCATGAGAATCAAG
GTGGTCGAGGGCGGCCCTCTCCCCTTCGCCTTCGACATCCTGGCTACTAGCTTCCTCTACGGC
AGCAAGACCTTCATCAACCACACCCAGGGCATCCCCGACTTCTTCAAGCAGTCCTTCCCTGA
GGGCTTCACATGGGAGAGAGTCACCACATACGAAGACGGGGGCGTGCTGACCGCTACCCAG
GACACCAGCCTCCAGGACGGCTGCCTCATCTACAACGTCAAGATCAGAGGGGTGAACTTCA
CATCCAACGGCCCTGTGATGCAGAAGAAAACACTCGGCTGGGAGGCCTTCACCGAGACGCT
GTACCCCGCTGACGGCGGCCTGGAAGGCAGAAACGACATGGCCCTGAAGCTCGTGGGCGGG
AGCCATCTGATCGCAAACATCAAGACCACATATAGATCCAAGAAACCCGCTAAGAACCTCA
AGATGCCTGGCGTCTACTATGTGGACTACAGACTGGAAAGAATCAAGGAGGCCAACAACGA
GACCTACGTCGAGCAGCACGAGGTGGCAGTGGCCAGATACTGCGACCTCCCTAGCAAACTG
GGGCACAAGCTTAATtAAGGGCCCGTTTAAACCCGCTGATCAGCCTCGACTGTGCCTTCTA
SEQ ID NO: 67 (see e.g., polypeptide in SEQ ID NO: 31) - miRFP670-ALFA
TAATACGACTCACTATAGGGAGACCCAAGCTGGCTAGCGTTTAAACTTAAGCTTatggtagcaggtc
atgcctctggcagccccgcattcgggaccgcctctcattcgaattgcgaacatgaagagatccacctcgccggctcgatccagccgcatggcgcgcttct
ggtcgtcagcgaacatgatcatcgcgtcatccaggccagcgccaacgccgcggaatttctgaatctcggaagcgtactcggcgttccgctcgccgagat
cgacggcgatctgttgatcaagatcctgccgcatctcgatcccaccgccgaaggcatgccggtcgcggtgcgctgccggatcggcaatccctctacgg
agtactgcggtctgatgcatcggcctccggaaggcgggctgatcatcgaactcgaacgtgccggcccgtcgatcgatctgtcaggcacgctggcgccg
gcgctggagcggatccgcacggcgggttcactgcgcgcgctgtgcgatgacaccgtgctgctgtttcagcagtgcaccggctacgaccgggtgatggt
gtatcgtttcgatgagcaaggccacggcctggtattctccgagtgccatgtgcctgggctcgaatcctatttcggcaaccgctatccgtcgtcgactgtccc
gcagatggcgcggcagctgtacgtgcggcagcgcgtccgcgtgctggtcgacgtcacctatcagccggtgccgctggagccgcggctgtcgccgctg
accgggcgcgatctcgacatgtcgggctgcttcctgcgctcgatgtcgccgtgccatctgcagttcctgaaggacatgggcgtgcgcgccaccctggcg
gtgtcgctggtggtcggcggcaagctgtggggcctggttgtctgtcaccattatctgccgcgcttcatccgtttcgagctgcgggcgatctgcaaacggct
cgccgaaaggatcgcgacgcggatcaccgcgcttgagagcCTCGAGTCCCGCCTGGAGGAAGAACTTCGGAGGAG
ACTTACTGAGtaatctagagggccctattctatagtgtcacctaaatgctagagctcgctgatcagcctcgactgtgccttcta
SEQ ID NO: 68 (see e.g., polypeptide in SEQ ID NO: 32) - MCP-linker-ADAR2-DDN-
SpyTag- ADAR2(E488Q)-DDC-TagBFP
TAATACGACTCACTATAGGGAGACCCAAGCTGGctagaggatcgaacccttaaggccaccatggcgtccaatttcactca
gtttgtgctggttgacaacgggggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCGGAGGTAGCggagcccacatcgtg
atggtggacgcctacaagccgacgaagGGAACCGGTGACagacatcctaataggaaggctagaggccaacttcggacgaagattgaaagt
ggccagggtactatcccggtgcggtccaacgctagtattcaaacgtgggacggagtccttcaaggtgaacggctgttgacaatgagctgctcagacaaa
atcgcgcgctggaatgtagtgggaatccaaggcagcctcttgagcatattcgtagaacccatatatttctcatccattattttgggctctctgtatcatggtgac
catctgtcaagggctatgtaccaacgaatttctaatatcgaggatcttcctccactctatacactcaataagcctctcttgtccgggatatcaaacgctgaggc
ccgccagccagggaaagctcctaacttcagtgttaactggaccgttggtgattctgcgatagaggtcatcaacgccacgacaggtaaggatgagctcggt
agagcctcacgcctgtgtaaacacgcgttgtattgtagatggatgagagtacatgggaaggtcccatctcacttgctccgaagcaagatcactaagcctaa
tgtgtatcatgagtcaaaactcgcggctaaagaataccaggcagccaaagctcgactttttacagcttttattaaggcagggctcggggcatgggtcgaga
agccgaccgagcaggaccaattctctctgacggggagcggatccAGCGAGCTGATTAAGGAGAACATGCACATGAAG
CTGTACATGGAGGGCACCGTGGACAACCATCACTTCAAGTGCACATCCGAGGGCGAAGGCA
AGCCCTACGAGGGCACCCAGACCATGAGAATCAAGGTGGTCGAGGGCGGCCCTCTCCCCTT
CGCCTTCGACATCCTGGCTACTAGCTTCCTCTACGGCAGCAAGACCTTCATCAACCACACCC
AGGGCATCCCCGACTTCTTCAAGCAGTCCTTCCCTGAGGGCTTCACATGGGAGAGAGTCACC
ACATACGAAGACGGGGGCGTGCTGACCGCTACCCAGGACACCAGCCTCCAGGACGGCTGCC
TCATCTACAACGTCAAGATCAGAGGGGTGAACTTCACATCCAACGGCCCTGTGATGCAGAA
GAAAACACTCGGCTGGGAGGCCTTCACCGAGACGCTGTACCCCGCTGACGGCGGCCTGGAA
GGCAGAAACGACATGGCCCTGAAGCTCGTGGGGGGAGCCATCTGATCGCAAACATCAAGA
CCACATATAGATCCAAGAAACCCGCTAAGAACCTCAAGATGCCTGGCGTCTACTATGTGGAC
TACAGACTGGAAAGAATCAAGGAGGCCAACAACGAGACCTACGTCGAGCAGCACGAGGTG
GCAGTGGCCAGATACTGCGACCTCCCTAGCAAACTGGGGCACAAGCTTAATtAAGGGCCCGT
TTAAACCCGCTGATCAGCCTCGACTGTGCCTTCTA
SEQ ID NO: 69 (see e.g., polypeptide in SEQ ID NO: 33) - MCP-linker-ADAR2-DDN-
SpyTag- ADAR2(E488Q)-DDC-SpyCatcher-TagBFP
TAATACGACTCACTATAGGGAGACCCAAGCTGGctagaggatcgaacccttaaggccaccatggcgtccaatttcactca
gtttgtgctggttgacaacggcgggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCGGAGGTAGCggagcccacatcgtg
atggtggacgcctacaagccgacgaagGGAACCGGTGACagacatcctaataggaaggctagaggccaacttcggacgaagattgaaagt
ggccagggtactatcccggtgcggtccaacgctagtattcaaacgtgggacggagtccttcaaggtgaacggctgttgacaatgagctgctcagacaaa
atcgcgcgctggaatgtagtgggaatccaaggcagcctcttgagcatattcgtagaacccatatatttctcatccattattttgggctctctgtatcatggtgac
catctgtcaagggctatgtaccaacgaatttctaatatcgaggatcttcctccactctatacactcaataagcctctcttgtccgggatatcaaacgctgaggc
ccgccagccagggaaagctcctaacttcagtgttaactggaccgttggtgattctgcgatagaggtcatcaacgccacgacaggtaaggatgagctcggt
agagcctcacgcctgtgtaaacacgcgttgtattgtagatggatgagagtacatgggaaggtcccatctcacttgctccgaagcaagatcactaagcctaa
tgtgtatcatgagtcaaaactcgcggctaaagaataccaggcagccaaagctcgactttttacagcttttattaaggcagggctcggggcatgggtcgaga
agccgaccgagcaggaccaattctctctgacggggagcGGTACCagcggaggcgccatggttgataccttatcaggtttatcaagtgagcaaggt
cagtccggtgatatgacaattgaagaagatagtgctacccatattaaattctcaaaacgtgatgaggacggcaaagagttagctggtgcaactatggagtt
gcgtgattcatctggtaaaactattagtacatggatttcagatggacaagtgaaagatttctacctgtatccaggaaaatatacatttgtcgaaaccgcagcac
cagacggttatgaggtagcaactgctattacctttacagttaatgagcaaggtcaggttactgtaaatggcaaagcaactaaaggtgacgctcatattggat
ccAGCGAGCTGATTAAGGAGAACATGCACATGAAGCTGTACATGGAGGGCACCGTGGACAA
CCATCACTTCAAGTGCACATCCGAGGGCGAAGGCAAGCCCTACGAGGGCACCCAGACCATG
AGAATCAAGGTGGTCGAGGGCGGCCCTCTCCCCTTCGCCTTCGACATCCTGGCTACTAGCTT
CCTCTACGGCAGCAAGACCTTCATCAACCACACCCAGGGCATCCCCGACTTCTTCAAGCAGT
CCTTCCCTGAGGGCTTCACATGGGAGAGAGTCACCACATACGAAGACGGGGGCGTGCTGAC
CGCTACCCAGGACACCAGCCTCCAGGACGGCTGCCTCATCTACAACGTCAAGATCAGAGGG
GTGAACTTCACATCCAACGGCCCTGTGATGCAGAAGAAAACACTCGGCTGGGAGGCCTTCA
CCGAGACGCTGTACCCCGCTGACGGCGGCCTGGAAGGCAGAAACGACATGGCCCTGAAGCT
CGTGGGCGGGAGCCATCTGATCGCAAACATCAAGACCACATATAGATCCAAGAAACCCGCT
AAGAACCTCAAGATGCCTGGCGTCTACTATGTGGACTACAGACTGGAAAGAATCAAGGAGG
CCAACAACGAGACCTACGTCGAGCAGCACGAGGTGGCAGTGGCCAGATACTGCGACCTCCC
TAGCAAACTGGGGCACAAGCTTAATtAAGGGCCCGTTTAAACCCGCTGATCAGCCTCGACTG
TGCCTTCTA
SEQ ID NO: 70 (see e.g., polypeptide in SEQ ID NO: 34) - MCP-linker-ADAR2-DDN-
SpyTag- ADAR2(E488Q)-DDC-TagBFP -P2A-T2A-SpyCatcher
TAATACGACTCACTATAGGGAGACCCAAGCTGGctagaggatcgaacccttaaggccaccatggcgtccaatttcactca
gtttgtgctggttgacaacggcgggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCGGAGGTAGCggagcccacatcgtg
atggtggacgcctacaagccgacgaagGGAACCGGTGACagacatcctaataggaaggctagaggccaacttcggacgaagattgaaagt
ggccagggtactatcccggtgcggtccaacgctagtattcaaacgtgggacggagtccttcaaggtgaacggctgttgacaatgagctgctcagacaaa
atcgcgcgctggaatgtagtgggaatccaaggcagcctcttgagcatattcgtagaacccatatatttctcatccattattttgggctctctgtatcatggtgac
catctgtcaagggctatgtaccaacgaatttctaatatcgaggatcttcctccactctatacactcaataagcctctcttgtccgggatatcaaacgctgaggc
ccgccagccagggaaagctcctaacttcagtgttaactggaccgttggtgattctgcgatagaggtcatcaacgccacgacaggtaaggatgagctcggt
agagcctcacgcctgtgtaaacacgcgttgtattgtagatggatgagagtacatgggaaggtcccatctcacttgctccgaagcaagatcactaagcctaa
tgtgtatcatgagtcaaaactcgcggctaaagaataccaggcagccaaagctcgactttttacagcttttattaaggcagggctcggggcatgggtcgaga
agccgaccgagcaggaccaattctctctgacggggagcggatccAGCGAGCTGATTAAGGAGAACATGCACATGAAG
CTGTACATGGAGGGCACCGTGGACAACCATCACTTCAAGTGCACATCCGAGGGCGAAGGCA
AGCCCTACGAGGGCACCCAGACCATGAGAATCAAGGTGGTCGAGGGCGGCCCTCTCCCCTT
CGCCTTCGACATCCTGGCTACTAGCTTCCTCTACGGCAGCAAGACCTTCATCAACCACACCC
AGGGCATCCCCGACTTCTTCAAGCAGTCCTTCCCTGAGGGCTTCACATGGGAGAGAGTCACC
ACATACGAAGACGGGGGCGTGCTGACCGCTACCCAGGACACCAGCCTCCAGGACGGCTGCC
TCATCTACAACGTCAAGATCAGAGGGGTGAACTTCACATCCAACGGCCCTGTGATGCAGAA
GAAAACACTCGGCTGGGAGGCCTTCACCGAGACGCTGTACCCCGCTGACGGCGGCCTGGAA
GGCAGAAACGACATGGCCCTGAAGCTCGTGGGCGGGAGCCATCTGATCGCAAACATCAAGA
CCACATATAGATCCAAGAAACCCGCTAAGAACCTCAAGATGCCTGGCGTCTACTATGTGGAC
TACAGACTGGAAAGAATCAAGGAGGCCAACAACGAGACCTACGTCGAGCAGCACGAGGTG
GCAGTGGCCAGATACTGCGACCTCCCTAGCAAACTGGGGCACAAGCTTAATACTAGTGCCAC
AAACTTCTCTCTGCTAAAGCAAGCAGGTGATGTTGAAGAAAACCCAGGGCCTGGAGGGTCC
GAGGGCAGGGGAAGTCTCCTAACATGCGGGGACGTGGAGGAAAATCCCGGACCAGGTACCa
gcggaggcgccatggttgataccttatcaggtttatcaagtgagcaaggtcagtccggtgatatgacaattgaagaagatagtgctacccatattaaattctc
aaaacgtgatgaggacggcaaagagttagctggtgcaactatggagttgcgtgattcatctggtaaaactattagtacatggatttcagatggacaagtga
aagatttctacctgtatccaggaaaatatacatttgtcgaaaccgcagcaccagacggttatgaggtagcaactgctattacctttacagttaatgagcaagg
tcaggttactgtaaatggcaaagcaactaaaggtgacgctcatattggatAAGGGCCCGTTTAAACCCGCTGATCAGCCTCG
ACTGTGCCTTCTA
SEQ ID NO: 71 (see e.g., polypeptide in SEQ ID NO: 35) - MCP-linker-ADAR2-DDN-
SpyTag- ADAR2(E488Q)-DDC-TEVcs-SpyCatcher-TagBFP
TAATACGACTCACTATAGGGAGACCCAAGCTGGctagaggatcgaacccttaaggccaccatggcgtccaatttcactca
gtttgtgctggttgacaacggcgggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCGGAGGTAGCggagcccacatcgtg
atggtggacgcctacaagccgacgaagGGAACCGGTGACagacatcctaataggaaggctagaggccaacttcggacgaagattgaaagt
ggccagggtactatcccggtgcggtccaacgctagtattcaaacgtgggacggagtccttcaaggtgaacggctgttgacaatgagctgctcagacaaa
atcgcgcgctggaatgtagtgggaatccaaggcagcctcttgagcatattcgtagaacccatatatttctcatccattattttgggctctctgtatcatggtgac
catctgtcaagggctatgtaccaacgaatttctaatatcgaggatcttcctccactctatacactcaataagcctctcttgtccgggatatcaaacgctgaggc
ccgccagccagggaaagctcctaacttcagtgttaactggaccgttggtgattctgcgatagaggtcatcaacgccacgacaggtaaggatgagctcggt
agagcctcacgcctgtgtaaacacgcgttgtattgtagatggatgagagtacatgggaaggtcccatctcacttgctccgaagcaagatcactaagcctaa
tgtgtatcatgagtcaaaactcgcggctaaagaataccaggcagccaaagctcgactttttacagcttttattaaggcagggctcggggcatgggtcgaga
agccgaccgagcaggaccaattctctctgacggggagcGGAGGTACGGAGAATTTGTATTTTCAGAGCGGTACCag
cggaggcgccatggttgataccttatcaggtttatcaagtgagcaaggtcagtccggtgatatgacaattgaagaagatagtgctacccatattaaattctca
aaacgtgatgaggacggcaaagagttagctggtgcaactatggagttgcgtgattcatctggtaaaactattagtacatggatttcagatggacaagtgaa
agatttctacctgtatccaggaaaatatacatttgtcgaaaccgcagcaccagacggttatgaggtagcaactgctattacctttacagttaatgagcaaggt
caggttactgtaaatggcaaagcaactaaaggtgacgctcatattggatccAGCGAGCTGATTAAGGAGAACATGCACATG
AAGCTGTACATGGAGGGCACCGTGGACAACCATCACTTCAAGTGCACATCCGAGGGCGAAG
GCAAGCCCTACGAGGGCACCCAGACCATGAGAATCAAGGTGGTCGAGGGCGGCCCTCTCCC
CTTCGCCTTCGACATCCTGGCTACTAGCTTCCTCTACGGCAGCAAGACCTTCATCAACCACAC
CCAGGGCATCCCCGACTTCTTCAAGCAGTCCTTCCCTGAGGGCTTCACATGGGAGAGAGTCA
CCACATACGAAGACGGGGGCGTGCTGACCGCTACCCAGGACACCAGCCTCCAGGACGGCTG
CCTCATCTACAACGTCAAGATCAGAGGGGTGAACTTCACATCCAACGGCCCTGTGATGCAGA
AGAAAACACTCGGCTGGGAGGCCTTCACCGAGACGCTGTACCCCGCTGACGGCGGCCTGGA
AGGCAGAAACGACATGGCCCTGAAGCTCGTGGGCGGGAGCCATCTGATCGCAAACATCAAG
ACCACATATAGATCCAAGAAACCCGCTAAGAACCTCAAGATGCCTGGCGTCTACTATGTGGA
CTACAGACTGGAAAGAATCAAGGAGGCCAACAACGAGACCTACGTCGAGCAGCACGAGGT
GGCAGTGGCCAGATACTGCGACCTCCCTAGCAAACTGGGGCACAAGCTTAATtAAGGGCCCG
TTTAAACCCGCTGATCAGCCTCGACTGTGCCTTCTA
SEQ ID NO: 72 (see e.g., polypeptide in SEQ ID NO: 36) - TEV Protease
Taatacgactcactatagggagacccaagctggctagttaagcttgccaccatgggcgagagccttttcaagggcccgagggactacaacccgatctcc
agcaccatctgtcacctgaccaacgagagcgacggtcacaccactagtctgtacggcatcggcttcggccccttcatcatcaccaacaagcatctgttca
ggaggaataacggcacactgctggtgcaaagcctgcacggcgtgttcaaagtgaagaacacaaccaccctgcaacagcacctgatcgacggcaggg
acatgattatcatcaggatgcccaaggacttccccccctttccccagaaactgaagttcagggagccacaaagggaggagcgaatctgcctggtgacca
ccaacttccagaccaagtccatgagcagcatggtctctgataccagctgcaccttccccagcagcgacggcatcttctggaagcactggattcagacgaa
ggatggccaatgcggcagcccattggtgagcactagggacggcttcatcgtgggcatccacagcgccagcaattttaccaataccaacaactacttcac
gagcgtgccgaaaaacttcatggagctgttgaccaatcaagaggcgcagcagtgggtgagcggctggaggctgaacgccgacagcgttctttggggc
ggacataaggtgttcatggtcaagcccgaggaacccttccagcccgttaaggaagccactcagctttgataactcgagtctagagggcccgcggttcga
acaaaaactcatctcagaagaggatctgaatatgcataccggtcatcatcaccatcaccattgagtttaaacccgctgatcagcctcgactgtgccttcta
SEQ ID NO: 73 (see e.g., polypeptide in SEQ ID NO: 37) - MCP-linker-ADAR2-DDN-
Bad(L)- ADAR2(E488Q)-DDC-PhoCl-Bcl-xL-TagBFP
TAATACGACTCACTATAGGGAGACCCAAGCTGGctagaggatcgaacccttaaggccaccatggcgtccaatttcactca
gtttgtgctggttgacaacggcgggaccggggacgttacggtagccccctcaaactttgccaacggtatagcggagtggataagcagcaattctaggag
tcaagcatacaaagttacatgcagcgtgcgccaatctagcgctcagaatcgcaagtacaccattaaagtagaggtccccaagggagcctggagaagcta
tcttaacatggagttgaccataccaatcttcgctaccaactctgactgtgaactcattgtgaaagccatgcaaggtctgctcaaggatggtaacccaattccg
tccgctatcgctgccaactctgggatttacgggggcagtgggagcggtgcaggatctggtagtccagctgggggaggagcaccgggtagcggtgggg
ggtctcagctgcacctgccccaggttctcgcagacgccgtatcccgccttgtactgggcaagtttggtgatcttactgacaatttttcatctcctcatgcgag
gcggaaagtactcgcaggcgtcgtcatgacgaccggaactgacgtgaaagacgccaaagtcatctctgtctccacgggcacaaagtgcataaacggg
gagtacatgagcgaccgggggctggcactgaatgattgtcacgctgaaataatatctaggcgatctctgcttagatttctctacactcaactcgaattgtacc
ttaacaacaaagatgaccagaaacgcagtatatttcagaaatcagaacgcggcggatttcgacttaaggaaaacgttcagttccacttgtatatcagcacat
ccccttgcggtgacgcccgaatcttttccccgcacgagccgatattggaggagcccgcgGCTAGCGGGTCGGGCACCGGTGCTC
CACCCAATCTCTGGGCAGCGCAGCGCTACGGCCGTGAGCTCAGAAGGATGTCCGATGAGcTG
GTCGACagacatcctaataggaaggctagaggccaacttcggacgaagattgaaagtggccagggtactatcccggtgcggtccaacgctagtatt
caaacgtgggacggagtccttcaaggtgaacggctgttgacaatgagctgctcagacaaaatcgcgcgctggaatgtagtgggaatccaaggcagcct
cttgagcatattcgtagaacccatatatttctcatccattattttgggctctctgtatcatggtgaccatctgtcaagggctatgtaccaacgaatttctaatatcg
aggatcttcctccactctatacactcaataagcctctcttgtccgggatatcaaacgctgaggcccgccagccagggaaagctcctaacttcagtgttaact
ggaccgttggtgattctgcgatagaggtcatcaacgccacgacaggtaaggatgagctcggtagagcctcacgcctgtgtaaacacgcgttgtattgtag
atggatgagagtacatgggaaggtcccatctcacttgctccgaagcaagatcactaagcctaatgtgtatcatgagtcaaaactcgcggctaaagaatacc
aggcagccaaagctcgactttttacagcttttattaaggcagggctcggggcatgggtcgagaagccgaccgagcaggaccaattctctctgacgggga
gcGGAAGTGGTggtGTGATCCCTGACTACTTCAAGCAGAGCTTCCCCGAGGGCTACAGCTGGG
AGCGCAGCATGACCTACGAGGACGGCGGCATCTGCATCGCCACCAACGACATCACAATGGA
GGGGGACAGCTTCATCAACAAGATCCACTTCAAGGGCACGAACTTCCCCCCCAACGGCCCC
GTGATGCAGAAGAGGACCGTGGGCTGGGAGGCCAGCACCGAGAAGATGTACGAGCGCGAC
GGCGTGCTGAAGGGCGACGTGAAGATGAAGCTGCTGCTGAAGGGCGGCGGCCACTATCGCT
GCGACTACCGCACCACCTACAAGGTCAAGCAGAAGCCCGTAAAGCTGCCCGACTACCACTT
CGTGGACCACCGCATCGAGATCCTGAGCCACGACAAGGACTACAACAAGGTGAAGCTGTAC
GAGCACGCCGTGGCCCGCAACTCCACCGACAGCATGGACGAGCTGTACAAGGGTGGCAGCG
GTGGCATGGTGAGCAAGGGCGAGGAGACCATTACAAGCGTGATCAAGCCTGACATGAAGAA
CAAGCTGCGCATGGAGGGCAACGTGAACGGCCACGCCTTCGTGATCGAGGGCGAGGGCAGC
GGCAAGCCCTTCGAGGGCATCCAGACGATTGATTTGGAGGTGAAGGAGGGCGCCCCGCTGC
CCTTCGCCTACGACATCCTGACCACCGCCTTCCACTACGGCAACCGCGTGTTCACCAAGTAC
CCACGGTCGGGAAGTGGCTCAAGTAACCGGGAGCTGGTGGTTGACTTTCTCTCCTACAAGCT
TTCCCAGAAAGGATACAGCTGGAGTCAGTTTAGTGATGTGGAAGAGAACAGGACTGAGGCC
CCAGAAGGGACTGAATCGGAGATGGAGACCCCCAGTGCCATCAATGGCAACCCATCCTGGC
ACCTGGCAGACAGCCCCGCGGTGAATGGAGCCACTGGCCACAGCAGCAGTTTGGATGCCCG
GGAGGTGATCCCCATGGCAGCAGTAAAGCAAGCGCTGAGGGAGGCAGGCGACGAGTTTGAA
CTGCGGTACCGGCGGGCATTCAGTGACCTGACATCCCAGCTCCACATCACCCCAGGGACAGC
ATATCAGAGCTTTGAACAGGTAGTGAATGAACTCTTCCGGGATGGGGTAAACTGGGGTCGC
ATTGTGGCCTTTTTCTCCTTCGGCGGGGCACTGTGCGTGGAAAGCGTAGACAAGGAGATGCA
GGTATTGGTGAGTCGGATCGCAGCTTGGATGGCCACTTACCTGAATGACCACCTAGAGCCTT
GGATCCAGGAGAACGGCGGCTGGGATACTTTTGTGGAACTCTATGGGAACAATggatccAGCG
AGCTGATTAAGGAGAACATGCACATGAAGCTGTACATGGAGGGCACCGTGGACAACCATCA
CTTCAAGTGCACATCCGAGGGCGAAGGCAAGCCCTACGAGGGCACCCAGACCATGAGAATC
AAGGTGGTCGAGGGCGGCCCTCTCCCCTTCGCCTTCGACATCCTGGCTACTAGCTTCCTCTAC
GGCAGCAAGACCTTCATCAACCACACCCAGGGCATCCCCGACTTCTTCAAGCAGTCCTTCCC
TGAGGGCTTCACATGGGAGAGAGTCACCACATACGAAGACGGGGGCGTGCTGACCGCTACC
CAGGACACCAGCCTCCAGGACGGCTGCCTCATCTACAACGTCAAGATCAGAGGGGTGAACT
TCACATCCAACGGCCCTGTGATGCAGAAGAAAACACTCGGCTGGGAGGCCTTCACCGAGAC
GCTGTACCCCGCTGACGGCGGCCTGGAAGGCAGAAACGACATGGCCCTGAAGCTCGTGGGC
GGGAGCCATCTGATCGCAAACATCAAGACCACATATAGATCCAAGAAACCCGCTAAGAACC
TCAAGATGCCTGGCGTCTACTATGTGGACTACAGACTGGAAAGAATCAAGGAGGCCAACAA
CGAGACCTACGTCGAGCAGCACGAGGTGGCAGTGGCCAGATACTGCGACCTCCCTAGCAAA
CTGGGGCACAAGCTTAATtAAGGGCCCGTTTAAACCCGCTGATCAGCCTCGACTGTGCCTTCT
A

### Example 5

### Homologous ADAR Sequences

The iAD approach described herein can work for other ADAR2 isoforms and homologous proteins such as ADAR1 and ADAR3 (although ADAR3 is a catalytically inactive). Without wishing to be bound by theory, it is hypothesized that all 3 ADARs bind IP6 and take on a similar structure.

In addition to these proteins, there are three other homologues encoded in mammalian genome which can be susceptible to a similar engineering: ADAT1, ADAD1, and ADAD2. ADAT1 is a tRNA-specific adenosine deaminase which is known to require IP6 and catalyzes the deamination of adenosine in the anticodon loop of tRNAs. ADAD1 and ADAD2 are two proteins with expression limited to male testis and are important for germ cell differentiation. They are thought to be catalytically inactive and mutation of them does not affect male germ cell RNA editing.

ADAR3, ADAD1, and ADAD2 are thought to be catalytically inactive; it is contemplated here that ADAR3, ADAD1, and/or ADAD2 can be engineered to be catalytically active (e.g., by inserting regions of or mutating residues to that of ADAR1, ADAR2, and/or ADAT1 that allow for such catalytic deaminase activity). ADAT1 edits conserved loop structures in tRNA; it is contemplated here that ADAT1 can be engineered to target dsRNA regions of mRNA (e.g., by inserting regions of or mutating residues to that of ADAR1, ADAR2, ADAR3, ADAD1, and/or ADAD2 that allow for such binding to dsRNA regions of mRNA). The ADAR2 constructs described herein can at least be adapted using ADAR1 or the engineered versions of ADAR3, ADAD1, ADAD2, and/or ADAT1.

Based on multiple sequence alignments and other groups' research, annotated in bold below is the deaminase domain of ADAR1(E1008Q) and bold-double-underlined is the 5′ RNA binding loop residues that can be amenable to insertion and the predicted insertion site with //(based on Park, S., Doherty, E. E., Xie, Y., Padyana, A. K., Fang, F., Zhang, Y., Karki, A., Lcbrilla, C. B., Siegel, J. B. & Beal, P. A. High-throughput mutagenesis reveals unique structural features of human ADAR1. Nat Commun 11, 5130 (2020).). Additionally, bolded and italicized are residues that may not be necessary on each terminus.

ADAR1-DD(E1008Q) (834-1226)
SEQ ID NO: 74,
PLTGSTFHDQIAMLSHRCFNTLTNSFQPSLLGRKILAAIIMKKDSED
MGVVVSLGTGNRCVKGDSLSLKGETVNDCHAEIISRRGFIRFLYSEL
MKYNSQTAKDSIFEPAKGGEKLQIKKTVSFHLYISTAPCGDGALFD
KSCS//DRAMEST//ESRHYPVFENPKQGKL
RTKVENGQGTIPVESSDIVPTWDGIRLGERLRTMSCSDKILRWNVLG
LQGALLTHFLQPIYLKSVTLGYLFSQGHLTRAICCRVTRDGSAFEDG
LRHPFIVNHPKVGRVSIYDSKRQSGKTKETSVNWCLADGYDLEILDG
TRGTVDGPRNELSRVSKKNIFLLFKKLCSFRYRRDLLRLSYGEAKKA
ARDYETAKNYFKKGLKDMGYGNWISKPQEEKNFYLCPV

Based on multiple sequence alignment (ADAR2 and ADAR3 are closely related), this is the deaminase domain for ADAR3.

ADAR3-DD(E527Q) (354-738)
SEQ ID NO: 75,
RTPMPQEFADSISQLVTQKFREVTTDLTPMHARHKALAGIVMTKGL
DARQAQVVALSSGTKCISGEHLSDQGLVVNDCHAEVVARRAFLHFL
YTQLELHLSKRREDSERSIFVRLKEGGYRLRENILFHLYVSTSPCG
DARLHSPYEITTDLHS//SKHLVRKFRGHLRTKIESGQGTVPVRGP
SAVQTWDGVLLGEQLITMSCTDKIARWNVLGLQGALLSHFVEPVYL
QSIVVGSLHHTGHLARVMSHRMEGVGQLPASYRHNRPLLSGVSDTE
ARQPGKSPPFSMNWVVGSADLEIINATTGRRSCGGPSRLCKHVLSA
RWARLYGRLSTRTPSPGDTPSMYCEAKLGAHTYQSVKQQLFKAFQK
AGLGTWVRKPPEQQQFLLT

Predicted RNA binding loop shown are in bold-double-underlined text below. The termini of the deaminase domains can also work for heterodimer domains using N and C terminal fusions.

ADAD1-DD (198-576)
SEQ ID NO: 76,
HYEGRHIQYAKISQIVKERFNQLISNRSEYLKYSSSLAAF
IIERAGQHEVVAIGTGEYNYSQDIKPDGRVLHDTHAVVTA
RRSLLRYFYRQLLLFYSKNPAMMEKSIFCTEPTSNLLTLK
QNINICLYMNQLPKGSAQIKSQL//RLNPHSISAFEANEE
LCLHVAVEGKIYLTVYCPKDGVNRISSMSSSDKLTRWEVL
GVQGALLSHFIQPVYISSILIGDGNCSDTRGLEIAIKQRV
DDALTSKLPMFYLVNRPHISLVPSAYPLQMNLEYKFLSLN
WAQGDVSLEIVDGLSGKITESSPFKSGMSMASRLCKAAML
SRFNLLAKEAKKELLEAGTYHAAKCMSASYQEAKCKLKSY
LQQHGYGSWIVKSPCIEQFNM
ADAD2-DD (200-583)
SEQ ID NO: 77,
SVENILTHEQRCAALVSAGFDLLLDERSPYWACKGTVAGV
ILEREIPRARGHVKEIYKLVALGTGSSCCAGWLEFSGQQL
HDCHGLVIARRALLRFLFRQLLLATQGGPKGKEQSVLAPQ
PGPGPPFTLKPRVFLHLYISNTPKGAARDIYL
PPTSEG//GLPHSPPMRLQAHVLGQLKPVCYVAPSLCDTH
VGCLSASDKLARWAVLGLGGALLAHLVSPLYSTSLILADS
CHDPPTLSRAIHTRPCLDSVLGPCLPPPYVRTALHLFAGP
PVAPSEPTPDTCRGLSLNWSLGDPGIEVVDVATGRVKANA
ALGPPSRLCKASFLRAFHQAARAVGKPYLLALKTYEAAKA
GPYQEARRQLSLLLDQQGLGAWPSKPLVGKFRN

For ADAT1, a non-homologous stretch in annotated in bold

ADAT1-DD (1-502)
SEQ ID NO: 78,
MWTADEIAQLCYEHYGIRLPKKGKPEPNHEWTLLAAVVKIQSPADKACDTPDKPVQVTKEVVS
MGTGTKCIGQSKMRKNGDILNDSHAEVIARRSFQRYLLHQLQLAATLKEDSIFVPGTQKGV
WKLRRDLIFVFFSSHTPCGDASIIPMLEFEDQPCCPVFRNWAHNSSVEASSNLEAPGNERKC
EDPDSPVTKKMRLEPGTAAREVTNGAAHHQSFGKQKSGPISPGIHSCDLTVEGLATVTRIAP
GSAKVIDVYRTGAKCVPGEAGDSGKPGAAFHQVGLLRVKPGRGDRTRSMSCSDKMARWNV
LGCQGALLMHLLEEPIYLSAVVIGKCPYSQEAMQRALIGRCQNVSALPKGFGVQELKILQSDLLF
EQSRSAVQAKRADSPGRLVPCGAAISWSAVPEQPLDVTANGFPQGTTKKTIGSLQARSQISKVEL
FRSFQKLLSRIARDKWPHSLRVQKLDTYQEYKEAASSYQEAWSTLRKQVFGSWIRNPPDYHQFK
P55265|DSRAD_HUMAN, ADAR1, 1226 amino acids (aa)
SEQ ID NO: 79,
MNPRQGYSLSGYYTHPFQGYEHRQLRYQQPGPGSSPSSFLLKQIEFLKGQLPEAPVIGKQTPSLPP
SLPGLRPRFPVLLASSTRGRQVDIRGVPRGVHLRSQGLQRGFQHPSPRGRSLPQRGVDCLSSHFQE
LSIYQDQEQRILKFLEELGEGKATTAHDLSGKLGTPKKEINRVLYSLAKKGKLQKEAGTPPLWKI
AVSTQAWNQHSGVVRPDGHSQGAPNSDPSLEPEDRNSTSVSEDLLEPFIAVSAQAWNQHSGVVR
PDSHSQGSPNSDPGLEPEDSNSTSALEDPLEFLDMAEIKEKICDYLFNVSDSSALNLAKNIGLTKA
RDINAVLIDMERQGDVYRQGTTPPIWHLTDKKRERMQIKRNTNSVPETAPAAIPETKRNAEFLTC
NIPTSNASNNMVTTEKVENGQEPVIKLENRQEARPEPARLKPPVHYNGPSKAGYVDFENGQWAT
DDIPDDLNSIRAAPGEFRAIMEMPSFYSHGLPRCSPYKKLTECQLKNPISGLLEYAQFASQTCEFN
MIEQSGPPHEPRFKFQVVINGREFPPAEAGSKKVAKQDAAMKAMTILLEEAKAKDSGKSEESSH
YSTEKESEKTAESQTPTPSATSFFSGKSPVTTLLECMHKLGNSCEFRLLSKEGPAHEPKFQYCVAV
GAQTFPSVSAPSKKVAKQMAAEEAMKALHGEATNSMASDNQPEGMISESLDNLESMMPNKVR
KIGELVRYLNTNPVGGLLEYARSHGFAAEFKLVDQSGPPHEPKFVYQAKVGGRWFPAVCAHSK
KQGKQEAADAALRVLIGENEKAERMGFTEVTPVTGASLRRTMLLLSRSPEAQPKTLPLTGSTFH
DQIAMLSHRCFNTLTNSFQPSLLGRKILAAIIMKKDSEDMGVVVSLGTGNRCVKGDSLSLKGETV
NDCHAEIISRRGFIRFLYSELMKYNSQTAKDSIFEPAKGGEKLQIKKTVSFHLYISTAPCGDGALFD
KSCSDRAMESTESRHYPVFENPKQGKLRTKVENGEGTIPVESSDIVPTWDGIRLGERLRTMSCSD
KILRWNVLGLQGALLTHFLQPIYLKSVTLGYLFSQGHLTRAICCRVTRDGSAFEDGLRHPFIVNHP
KVGRVSIYDSKRQSGKTKETSVNWCLADGYDLEILDGTRGTVDGPRNELSRVSKKNIFLLFKKL
CSFRYRRDLLRLSYGEAKKAARDYETAKNYFKKGLKDMGYGNWISKPQEEKNFYLCPV
P78563-2|RED1_HUMAN, ADAR2, 701 aa
SEQ ID NO: 80,
MDIEDEENMSSSSTDVKENRNLDNVSPKDGSTPGPGEGSQLSNGGGGGPGRKRPLEEGSNGHSK
YRLKKRRKTPGPVLPKNALMQLNEIKPGLQYTLLSQTGPVHAPLFVMSVEVNGQVFEGSGPTKK
KAKLHAAEKALRSFVQFPNASEAHLAMGRTLSVNTDFTSDQADFPDTLFNGFETPDKAEPPFYV
GSNGDDSFSSSGDLSLSASPVPASLAQPPLPVLPPFPPPSGKNPVMILNELRPGLKYDFLSESGESH
AKSFVMSVVVDGQFFEGSGRNKKLAKARAAQSALAAIFNLHLDQTPSRQPIPSEGLQLHLPQVL
ADAVSRLVLGKFGDLTDNFSSPHARRKVLAGVVMTTGTDVKDAKVISVSTGTKCINGEYMSDR
GLALNDCHAEIISRRSLLRFLYTQLELYLNNKDDQKRSIFQKSERGGFRLKENVQFHLYISTSPCG
DARIFSPHEPILEEPADRHPNRKARGQLRTKIESGEGTIPVRSNASIQTWDGVLQGERLLTMSCSD
KIARWNVVGIQGSLLSIFVEPIYFSSIILGSLYHGDHLSRAMYQRISNIEDLPPLYTLNKPLLSGISN
AEARQPGKAPNFSVNWTVGDSAIEVINATTGKDELGRASRLCKHALYCRWMRVHGKVPSHLLR
SKITKPNVYHESKLAAKEYQAAKARLFTAFIKAGLGAWVEKPTEQDQFSLTP
Q9NS39|RED2_HUMAN, ADAR3, 739 aa
SEQ ID NO: 81,
MASVLGSGRGSGGLSSQLKCKSKRRRRRRSKRKDKVSILSTFLAPFKHLSPGITNTEDDDTLSTSS
AEVKENRNVGNLAARPPPSGDRARGGAPGAKRKRPLEEGNGGHLCKLQLVWKKLSWSVAPKN
ALVQLHELRPGLQYRTVSQTGPVHAPVFAVAVEVNGLTFEGTGPTKKKAKMRAAELALRSFVQ
FPNACQAHLAMGGGPGPGTDFTSDQADFPDTLFQEFEPPAPRPGLAGGRPGDAALLSAAYGRRR
LLCRALDLVGPTPATPAAPGERNPVVLLNRLRAGLRYVCLAEPAERRARSFVMAVSVDGRTFEG
SGRSKKLARGQAAQAALQELFDIQMPGHAPGRARRTPMPQEFADSISQLVTQKFREVTTDLTPM
HARHKALAGIVMTKGLDARQAQVVALSSGTKCISGEHLSDQGLVVNDCHAEVVARRAFLHFLY
TQLELHLSKRREDSERSIFVRLKEGGYRLRENILFHLYVSTSPCGDARLHSPYEITTDLHSSKHLVR
KFRGHLRTKIESGEGTVPVRGPSAVQTWDGVLLGEQLITMSCTDKIARWNVLGLQGALLSHFVE
PVYLQSIVVGSLHHTGHLARVMSHRMEGVGQLPASYRHNRPLLSGVSDAEARQPGKSPPFSMN
WVVGSADLEIINATTGRRSCGGPSRLCKHVLSARWARLYGRLSTRTPSPGDTPSMYCEAKLGAH
TYQSVKQQLFKAFQKAGLGTWVRKPPEQQQFLLTL
Q96M93|ADAD1_HUMAN, 576 aa
SEQ ID NO: 82,
MASNNHWFQSSQVPSFAQMLKKNLPVQPATKTITTPTGWSSESYGLSKMASKVTQVTGNFPEPL
LSKNLSSISNPVLPPKKIPKEFIMKYKRGEINPVSALHQFAQMQRVQLDLKETVTTGNVMGPYFA
FCAVVDGIQYKTGLGQNKKESRSNAAKLALDELLQLDEPEPRILETSGPPPFPAEPVVLSELAYVS
KVHYEGRHIQYAKISQIVKERFNQLISNRSEYLKYSSSLAAFIIERAGQHEVVAIGTGEYNYSQDIK
PDGRVLHDTHAVVTARRSLLRYFYRQLLLFYSKNPAMMEKSIFCTEPTSNLLTLKQNINICLYMN
QLPKGSAQIKSQLRLNPHSISAFEANEELCLHVAVEGKIYLTVYCPKDGVNRISSMSSSDKLTRWE
VLGVQGALLSHFIQPVYISSILIGDGNCSDTRGLEIAIKQRVDDALTSKLPMFYLVNRPHISLVPSA
YPLQMNLEYKFLSLNWAQGDVSLEIVDGLSGKITESSPFKSGMSMASRLCKAAMLSRFNLLAKE
AKKELLEAGTYHAAKCMSASYQEAKCKLKSYLQQHGYGSWIVKSPCIEQFNM
Q8NCV1|ADAD2_HUMAN, 583 aa
SEQ ID NO: 83,
MASASQGADDDGSRRKPRLAASLQISPQPRPWRPLPAQAQSAWGPAPAPATYRAEGGWPQVSV
LRDSGPGAGAGVGELGAARAWENLGEQMGKAPRVPVPPAGLSLPLKDPPASQAVSLLTEYAAS
LGIFLLFREDQPPGPCFPFSVSAELDGVVCPAGTANSKTEAKQQAALSALCYIRSQLENPESPQTSS
RPPLAPLSVENILTHEQRCAALVSAGFDLLLDERSPYWACKGTVAGVILEREIPRARGHVKEIYKL
VALGTGSSCCAGWLEFSGQQLHDCHGLVIARRALLRFLFRQLLLATQGGPKGKEQSVLAPQPGP
GPPFTLKPRVFLHLYISNTPKGAARDIYLPPTSEGGLPHSPPMRLQAHVLGQLKPVCYVAPSLCDT
HVGCLSASDKLARWAVLGLGGALLAHLVSPLYSTSLILADSCHDPPTLSRAIHTRPCLDSVLGPC
LPPPYVRTALHLFAGPPVAPSEPTPDTCRGLSLNWSLGDPGIEVVDVATGRVKANAALGPPSRLC
KASFLRAFHQAARAVGKPYLLALKTYEAAKAGPYQEARRQLSLLLDQQGLGAWPSKPLVGKFR
N
Q9BUB4|ADAT1_HUMAN, 502 aa
SEQ ID NO: 84,
MWTADEIAQLCYEHYGIRLPKKGKPEPNHEWTLLAAVVKIQSPADKACDTPDKPVQVTKEVVS
MGTGTKCIGQSKMRKNGDILNDSHAEVIARRSFQRYLLHQLQLAATLKEDSIFVPGTQKGVWKL
RRDLIFVFFSSHTPCGDASIIPMLEFEDQPCCPVFRNWAHNSSVEASSNLEAPGNERKCEDPDSPV
TKKMRLEPGTAAREVTNGAAHHQSFGKQKSGPISPGIHSCDLTVEGLATVTRIAPGSAKVIDVYR
TGAKCVPGEAGDSGKPGAAFHQVGLLRVKPGRGDRTRSMSCSDKMARWNVLGCQGALLMHL
LEEPIYLSAVVIGKCPYSQEAMQRALIGRCQNVSALPKGFGVQELKILQSDLLFEQSRSAVQAKR
ADSPGRLVPCGAAISWSAVPEQPLDVTANGFPQGTTKKTIGSLQARSQISKVELFRSFQKLLSRIA
RDKWPHSLRVQKLDTYQEYKEAASSYQEAWSTLRKQVFGSWIRNPPDYHQFK

### Example 6

### Use Cases of ADAR Technology

### Diagnostic/Detection of Antigens in Vitro.

The modularity of the allosteric ADAR platform allows for rapid detection of antigens of interest via cell-free translation. In this case, the cis-heterodimerizing components are composed of an antibody fragment and an epitope (peptide or protein) of equal or lower affinity than that of the natural antigen. The allosteric-ADAR is either already present in the cell-free translation mixture, or is encoded as DNA and is added to a cell-free transcription and translation mixture. Additionally, the stop-codon editing reporter (fluorescent, luminescent, or colorimetric) is encoded by DNA and is also added by the user (along with a variable concentration of IP6). A sample of interest, the reporter DNA, IP6, and the ADAR DNA is mixed at time zero. If the sample contains the antigen of interest, the ADAR enzyme will become active due to the antibody fragment binding the soluble antigen, edit the reporter mRNA and lead to translation of a protein which can be read out (e.g., fluorescently, luminescently, or colorimetrically). In this way, signal can accumulate relatively rapidly.

One advantage that this system has is that by exogenously controlling the IP6 concentration added, one can shift the equilibrium of active vs. inactive ADAR and therefore tune the stringency of detection. One can use low affinity heterodimer pairs without necessarily increasing background if one also adds low concentrations of IP6. With this, it can be easier to adapt to different antibody fragments and it can be possible to detect low concentrations of antigen.

As well, because the reporter can be configured in a ratiometric way (upstream translation of one component vs. the downstream translation of another after editing), it can serve as an internal control and be easier to get an estimate of ADAR activity/antigen concentration.

It can be possible to increase the sensitivity of this system by encoding two antibody fragments which bind distinct epitopes of the same antigen. In this case, the sensor is autoinhibited only by one antibody-epitope pair, allowing the free antibody fragment to bind and concentrate the antigen. This increase in local concentration would lead to increased activation (and more sensitive detection). This can also be configured in a way to detect protease activity in a sample, or can be coupled with inducible proteolytic activity as another way to activate this cell-free circuit.

### Therapeutic Applications Via Antigen/Protease/Enzyme Activated RNA Circuits

The allosteric ADAR platform enables the ability for a single, easily deliverable mRNA to sense the intracellular environment and turn on or off a therapeutic payload accordingly. In this case, an mRNA encoding both the sensor and the actuator is delivered systemically (or specifically via a decorated LNP), and the therapeutic protein is translated only in diseased cells. The allosteric ADAR(s) that the mRNA encodes can be activated by disease-associated antigens, proteases, and/or protein activity.

For disease associated antigens, the ADAR is autoinhibited due to a cis-interacting heterodimeric pair consisting of an antibody-fragment and an epitope/epitope mimic with equal or lesser affinity than the natural antigen. When translated in host cells, the ADAR can remain in an inactive state unless the antigen of interest is present in cells. In this case, the higher affinity antigen preferentially binds to the antibody fragment, relieving the autoinhibition. This leads to the editing of the stop-codon and delivery of the therapeutic payload.

In this case, the use of two antibody fragments can increase the sensitivity of the system.

For example, such a construct can comprise an autoinhibited ADAR comprising a heterodimeric nanobody-epitope pair specific for the HIV protein p24. In this case, the construct comprises an N-terminal fusion of an attenuated p24 fragment and C-terminal fusion of two nanobodies specific for distinct epitopes (one of which is mutated so that the nanobody cannot bind). This can only turn on in HIV producing cells.

Alternative examples include encoding antibody-epitope pairs that recognize/mimic oncogenic proteins such as the HPV protein E6.

For disease associated proteases, the ADAR is autoinhibited due to a constitutively interacting heterodimer pair where one component contains a protease cut site in the linker between the ADAR C-terminus and the dimer component N-terminus. This ADAR can be repressed unless there is a cleavage event, at which point it can spontaneously activate and turn on the translation of the payload.

An example, such a construct can comprise the ALFA nanobody/epitope pair with an HIV PR cut site in the linker between the nanobody and the ADAR. In this case, only in cells where there is active HIV PR can the ADAR turn on, limiting payload delivery to HIV producing/receiving cells.

For disease associated protein activity, the ADAR is autoinhibited due to a heterodimeric pair that be dissociated by competitive binding to the product of a disease associated enzyme.

As an example, the heterodimer can comprise a phosphotyrosine binding (PTB) domain and a peptide with moderate affinity for the phosphotyrosine domain. In the absence of cancer-associated constitutive signaling, the ADAR is repressed. However, in cancer cells where there is increased activity of receptor tyrosine kinases (RTKs), the ADAR is activated by the higher affinity interaction between the PTB and cancer-associated receptor.

See following reference for examples of circuit that exploits PTB; the contents of which are incorporated herein by reference in its entirety. Chung, H. K. et al. A compact synthetic pathway rewires cancer signaling to therapeutic effector release. Science eaat6982 (2019) doi: 10.1126/science.aat6982.

An additional PTB or SH2 domain can be added to localize autoinhibited ADAR to RTK.

In each of the preceding cases, the opposite logic can be applied using the ADAR-OFF circuit design to ensure that the ADAR activity leads to degradation of mRNA in the wrong cell type.

Additionally, one can combine multiple ADAR sensors and RNA circuit types into a single mRNA circuit. In this case, the presence of multiple factors would be necessary to turn on (or off) a payload. For example, a redundant circuit using p24-antigen sensing AND HIV PR sensing can ensure the expression is confined to the correct cells.

Precision Control of Timing and Extent of mRNA Translation or Degradation

Another exemplary use case outlined here is the ability to use the chemogenetically activated ADAR domains to precisely control when a therapeutic mRNA is translated or destroyed. In this case, instead of the ADAR sensor reacting to the cellular environment, it is reacting to an exogenously added small molecule drug. In this case, the ADAR is repressed by a heterodimer pair that can be dissociated by a small molecule, leading to an active ADAR domain.

The ADAR-OFF circuit can be implemented to quickly turn-off protein translation and destroy mRNA transcripts.

This can be a safety measure.

The ADAR-ON circuit can be implemented to tune the expression levels of a protein dependent on dosing in a small molecule drug.

These systems can also be used in tandem with two different drug-responsive ADARs: ADAR-ON allows dosage based control. ADAR-OFF allows quick turnover after initial.

### Example 7

### N-Terminal Fusion Site Works as an Alternative to Loop Insertion

The constructs described in above examples used engineered ADAR2 deaminase domains that all contained the same basic topology (FIG. 11a). In short, one component of a protein-based heterodimer was fused to the C-terminus of ADAR2-DD and one component (commonly a peptide) was inserted between residues A468 and D469. The inventors sought to determine the mechanism of autoinhibition. When the inventors inserted the BAD peptide into the ADAR deaminase domain (DD), they found that fusing its heterodimeric partner to the C-terminus was required for autoinhibition and that fusion to the N-terminus with a suitable linker did not inhibit the enzymatic activity (FIG. 11b). Based on the crystal structure of ADAR2-DD (FIG. 11c), the distance between the C-terminus and the insertion loop for the folded enzyme should be greater than 50 Å and should be incompatible with the domains interacting in cis.

The C-terminal portion of the deaminase domain is involved in binding the cofactor inositol hexaphosphate (IP6). This cofactor is required for the proper folding of catalytically active ADAR domains. Without wishing to be bound by theory, it was hypothesized that the autoinhibition seen in the engineered ADAR domains is due to the cis-interaction between the dimerization domains constraining the formation of the C-terminal IP6 binding pocket. In this hypothesis, the cis-interactions are more thermodynamically favorable than the formation and binding of IP6; drug addition interferes with the cis-heterodimer and shifts the equilibrium back towards the IP6-bound state.

A consequence of this hypothesis is that the fusion/insertion position of one of the dimer components may be variable, e.g., the only requirements are that one component is fused to the C-terminus and the other component is fused to a position far enough away to make the cis-interaction and IP6 binding mutually exclusive. Towards that end, the inventors tested if fusion to the N-terminus can substitute for the insertion site (FIG. 11d). The N-terminal Q316 residue is located on the opposite side of the folded ADAR domain and is over 50 Å away from the C-terminal T700 residue (FIG. 11e). The inventors found that the fusion of the N-terminal BAD peptide did not affect ADAR editing of the reporter construct on its own, but that Bcl-xL fusion to the C-terminus led to drug-dependent allostery (FIG. 11f). The background in the absence of drug was significantly higher in this conformation than in the previously described conformation, but the N-terminal site can serve as a more modular fusion site and can accommodate folded/globular heterodimeric protein partners more readily than the insertion site previously described. As well, this provides evidence for the IP6-competition model.

TABLE 2
Amino Acid Sequence Table
(see e.g., FIG. 11, 14)

| | SEQ | See | |
|---|---|---|---|
| | ID | e.g., | |
| NAME | NO | FIGS. | AMINO ACID SEQUENCE |
| MCP-linker- | 88 | 11b, | MASNFTQFVLVDNGGTGDVT |
| ADAR2- | | 11f | VAPSNFANGIAEWISSNSRS |
| DDN- | | | QAYKVTCSVRQSSAQNRKYT |
| Bad(L)- | | | IKVEVPKGAWRSYLNMELTI |
| ADAR2(E4 | | | PIFATNSDCELIVKAMQGLL |
| 88Q)-DDC- | | | KDGNPIPSAIAANSGIYGGS |
| TagBFP | | | GSGAGSGSPAGGGAPGSGGG |
| Also known | | | SQLHLPQVLADAVSRLVLGK |
| as “nDD- | | | FGDLTDNFSSPHARRKVLAG |
| BAD-CDD” | | | VVMTTGTDVKDAKVISVSTG |
| or “BAD(L) | | | TKCINGEYMSDRGLALNDCH |
| Only” | | | AEIISRRSLLRFLYTQLELY |
| | | | LNNKDDQKRSIFQKSERGGF |
| | | | RLKENVQFHLYISTSPCGDA |
| | | | RIFSPHEPILEEPAASGSGT |
| | | | GAPPNLWAAQRYGRELRRMS |
| | | | DELVDRHPNRKARGQLRTKI |
| | | | ESGQGTIPVRSNASIQTWDG |
| | | | VLQGERLLTMSCSDKIARWN |
| | | | VVGIQGSLLSIFVEPIYFSS |
| | | | IILGSLYHGDHLSRAMYQRI |
| | | | SNIEDLPPLYTLNKPLLSGI |
| | | | SNAEARQPGKAPNFSVNWTV |
| | | | GDSAIEVINATTGKDELGRA |
| | | | SRLCKHALYCRWMRVHGKVP |
| | | | SHLLRSKITKPNVYHESKLA |
| | | | AKEYQAAKARLFTAFIKAGL |
| | | | GAWVEKPTEQDQFSLTGSGS |
| | | | SELIKENMHMKLYMEGTVDN |
| | | | HHFKCTSEGEGKPYEGTQTM |
| | | | RIKVVEGGPLPFAFDILATS |
| | | | FLYGSKTFINHTQGIPDFFK |
| | | | QSFPEGFTWERVTTYEDGGV |
| | | | LTATQDTSLQDGCLIYNVKI |
| | | | RGVNFTSNGPVMQKKTLGWE |
| | | | AFTETLYPADGGLEGRNDMA |
| | | | LKLVGGSHLIANIKTTYRSK |
| | | | KPAKNLKMPGVYYVDYRLER |
| | | | IKEANNETYVEQHEVAVARY |
| | | | CDLPSKLGHKLN* |
| MCP-linker- | 89 | 11b | MASNFTQFVLVDNGGTGDVT |
| BclxL- | | | VAPSNFANGIAEWISSNSRS |
| linker- | | | QAYKVTCSVRQSSAQNRKYT |
| ADAR2- | | | IKVEVPKGAWRSYLNMELTI |
| DDN- | | | PIFATNSDCELIVKAMQGLL |
| Bad(L)- | | | KDGNPIPSAIAANSGIYGGS |
| ADAR2(E4 | | | GSGAGSGSPAGGGAPGSGGG |
| 88Q)-DDC | | | SQSNRELVVDFLSYKLSQKG |
| -TagBFP | | | YSWSQFSDVEENRTEAPEGT |
| Also known | | | ESEMETPSAINGNPSWHLAD |
| as “BclxL- | | | SPAVNGATGHSSSLDAREVI |
| nDD-BAD- | | | PMAAVKQALREAGDEFELRY |
| CDD” | | | RRAFSDLTSQLHITPGTAYQ |
| | | | SFEQVVNELFRDGVNWGRIV |
| | | | AFFSFGGALCVESVDKEMQV |
| | | | LVSRIAAWMATYLNDHLEPW |
| | | | IQENGGWDTFVELYGNNAAG |
| | | | GSGGSGGSGGSAAAQLHLPQ |
| | | | VLADAVSRLVLGKFGDLTDN |
| | | | FSSPHARRKVLAGVVMTTGT |
| | | | DVKDAKVISVSTGTKCINGE |
| | | | YMSDRGLALNDCHAEIISRR |
| | | | SLLRFLYTQLELYLNNKDDQ |
| | | | KRSIFQKSERGGFRLKENVQ |
| | | | FHLYISTSPCGDARIFSPHE |
| | | | PILEEPAASGSGTGAPPNLW |
| | | | AAQRYGRELRRMSDELVDRH |
| | | | PNRKARGQLRTKIESGQGTI |
| | | | PVRSNASIQTWDGVLQGERL |
| | | | LTMSCSDKIARWNVVGIQGS |
| | | | LLSIFVEPIYFSSIILGSLY |
| | | | HGDHLSRAMYQRISNIEDLP |
| | | | PLYTLNKPLLSGISNAEARQ |
| | | | PGKAPNFSVNWTVGDSAIEV |
| | | | INATTGKDELGRASRLCKHA |
| | | | LYCRWMRVHGKVPSHLLRSK |
| | | | ITKPNVYHESKLAAKEYQAA |
| | | | KARLFTAFIKAGLGAWVEKP |
| | | | TEQDQFSLTGSGSSELIKEN |
| | | | MHMKLYMEGTVDNHHFKCTS |
| | | | EGEGKPYEGTQTMRIKVVEG |
| | | | GPLPFAFDILATSFLYGSKT |
| | | | FINHTQGIPDFFKQSFPEGF |
| | | | TWERVTTYEDGGVLTATQDT |
| | | | SLQDGCLIYNVKIRGVNFTS |
| | | | NGPVMQKKTLGWEAFTETLY |
| | | | PADGGLEGRNDMALKLVGGS |
| | | | HLIANIKTTYRSKKPAKNLK |
| | | | MPGVYYVDYRLERIKEANNE |
| | | | TYVEQHEVAVARYCDLPSKL |
| | | | GHKLN* |
| MCP-linker- | 90 | 11b, 11f | MASNFTQFVLVDNGGTGDVT |
| ADAR2- | | | VAPSNFANGIAEWISSNSRS |
| DDN- | | | QAYKVTCSVRQSSAQNRKYT |
| Bad(L)- | | | IKVEVPKGAWRSYLNMELTI |
| ADAR2(E4 | | | PIFATNSDCELIVKAMQGLL |
| 88Q)-DDC- | | | KDGNPIPSAIAANSGIYGGS |
| Bcl-xL- | | | GSGAGSGSPAGGGAPGSGGG |
| TagBFP | | | SQLHLPQVLADAVSRLVLGK |
| Also known | | | FGDLTDNFSSPHARRKVLAG |
| as “nDD- | | | VVMTTGTDVKDAKVISVSTG |
| BAD-CDD- | | | TKCINGEYMSDRGLALNDCH |
| Bcl-xL” | | | AEIISRRSLLRFLYTQLELY |
| and | | | LNNKDDQKRSIFQKSERGGF |
| “BAD(L)” | | | RLKENVQFHLYISTSPCGDA |
| | | | RIFSPHEPILEEPAASGSGT |
| | | | GAPPNLWAAQRYGRELRRMS |
| | | | DELVDRHPNRKARGQLRTKI |
| | | | ESGQGTIPVRSNASIQTWDG |
| | | | VLQGERLLTMSCSDKIARWN |
| | | | VVGIQGSLLSIFVEPIYFSS |
| | | | IILGSLYHGDHLSRAMYQRI |
| | | | SNIEDLPPLYTLNKPLLSGI |
| | | | SNAEARQPGKAPNFSVNWTV |
| | | | GDSAIEVINATTGKDELGRA |
| | | | SRLCKHALYCRWMRVHGKVP |
| | | | SHLLRSKITKPNVYHESKLA |
| | | | AKEYQAAKARLFTAFIKAGL |
| | | | GAWVEKPTEQDQFSLTGSAA |
| | | | GGSGGSAAASSNRELVVDFL |
| | | | SYKLSQKGYSWSQFSDVEEN |
| | | | RTEAPEGTESEMETPSAING |
| | | | NPSWHLADSPAVNGATGHSS |
| | | | SLDAREVIPMAAVKQALREA |
| | | | GDEFELRYRRAFSDLTSQLH |
| | | | ITPGTAYQSFEQVVNELFRD |
| | | | GVNWGRIVAFFSFGGALCVE |
| | | | SVDKEMQVLVSRIAAWMATY |
| | | | LNDHLEPWIQENGGWDTFVE |
| | | | LYGNNGSSELIKENMHMKLY |
| | | | MEGTVDNHHFKCTSEGEGKP |
| | | | YEGTQTMRIKVVEGGPLPFA |
| | | | FDILATSFLYGSKTFINHTQ |
| | | | GIPDFFKQSFPEGFTWERVT |
| | | | TYEDGGVLTATQDTSLQDGC |
| | | | LIYNVKIRGVNFTSNGPVMQ |
| | | | KKTLGWEAFTETLYPADGGL |
| | | | EGRNDMALKLVGGSHLIANI |
| | | | KTTYRSKKPAKNLKMPGVYY |
| | | | VDYRLERIKEANNETYVEQH |
| | | | EVAVARYCDLPSKLGHKLN* |
| MCP-linker- | 91 | 11f | MASNFTQFVLVDNGGTGDVT |
| BAD- | | | VAPSNFANGIAEWISSNSRS |
| ADAR2- | | | QAYKVTCSVRQSSAQNRKYT |
| DD(E488Q) | | | IKVEVPKGAWRSYLNMELTI |
| -TagBFP | | | PIFATNSDCELIVKAMQGLL |
| Also known | | | KDGNPIPSAIAANSGIYGGS |
| as “BAD- | | | GSGAGSGSPAGGGAPGSGGG |
| DD” | | | STGAPPNLWAAQRYGRELRR |
| | | | MSDEFVDSFKKASQLHLPQV |
| | | | LADAVSRLVLGKFGDLTDNF |
| | | | SSPHARRKVLAGVVMTTGTD |
| | | | VKDAKVISVSTGTKCINGEY |
| | | | MSDRGLALNDCHAEIISRRS |
| | | | LLRFLYTQLELYLNNKDDQK |
| | | | RSIFQKSERGGFRLKENVQF |
| | | | HLYISTSPCGDARIFSPHEP |
| | | | ILEEPADRHPNRKARGQLRT |
| | | | KIESGQGTIPVRSNASIQTW |
| | | | DGVLQGERLLTMSCSDKIAR |
| | | | WNVVGIQGSLLSIFVEPIYF |
| | | | SSIILGSLYHGDHLSRAMYQ |
| | | | RISNIEDLPPLYTLNKPLLS |
| | | | GISNAEARQPGKAPNFSVNW |
| | | | TVGDSAIEVINATTGKDELG |
| | | | RASRLCKHALYCRWMRVHGK |
| | | | VPSHLLRSKITKPNVYHESK |
| | | | LAAKEYQAAKARLFTAFIKA |
| | | | GLGAWVEKPTEQDQFSLTGS |
| | | | GSSELIKENMHMKLYMEGTV |
| | | | DNHHFKCTSEGEGKPYEGTQ |
| | | | TMRIKVVEGGPLPFAFDILA |
| | | | TSFLYGSKTFINHTQGIPDF |
| | | | FKQSFPEGFTWERVTTYEDG |
| | | | GVLTATQDTSLQDGCLIYNV |
| | | | KIRGVNFTSNGPVMQKKTLG |
| | | | WEAFTETLYPADGGLEGRND |
| | | | MALKLVGGSHLIANIKTTYR |
| | | | SKKPAKNLKMPGVYYVDYRL |
| | | | ERIKEANNETYVEQHEVAVA |
| | | | RYCDLPSKLGHKLN* |
| MCP-linker- | 92 | 11f | MASNFTQFVLVDNGGTGDVT |
| BAD- | | | VAPSNFANGIAEWISSNSRS |
| ADAR2- | | | QAYKVTCSVRQSSAQNRKYT |
| DD(E488Q) | | | IKVEVPKGAWRSYLNMELTI |
| -Bcl-xL- | | | PIFATNSDCELIVKAMQGLL |
| TagBFP | | | KDGNPIPSAIAANSGIYGGS |
| Also known | | | GSGAGSGSPAGGGAPGSGGG |
| as “BAD- | | | STGAPPNLWAAQRYGRELRR |
| DD-BclxL” | | | MSDEFVDSFKKASQLHLPQV |
| and “WT” | | | LADAVSRLVLGKFGDLTDNF |
| | | | SSPHARRKVLAGVVMTTGTD |
| | | | VKDAKVISVSTGTKCINGEY |
| | | | MSDRGLALNDCHAEIISRRS |
| | | | LLRFLYTQLELYLNNKDDQK |
| | | | RSIFQKSERGGFRLKENVQF |
| | | | HLYISTSPCGDARIFSPHEP |
| | | | ILEEPADRHPNRKARGQLRT |
| | | | KIESGQGTIPVRSNASIQTW |
| | | | DGVLQGERLLTMSCSDKIAR |
| | | | WNVVGIQGSLLSIFVEPIYF |
| | | | SSIILGSLYHGDHLSRAMYQ |
| | | | RISNIEDLPPLYTLNKPLLS |
| | | | GISNAEARQPGKAPNFSVNW |
| | | | TVGDSAIEVINATTGKDELG |
| | | | RASRLCKHALYCRWMRVHGK |
| | | | VPSHLLRSKITKPNVYHESK |
| | | | LAAKEYQAAKARLFTAFIKA |
| | | | GLGAWVEKPTEQDQFSLTGS |
| | | | AAASSNRELVVDFLSYKLSQ |
| | | | KGYSWSQFSDVEENRTEAPE |
| | | | GTESEMETPSAINGNPSWHL |
| | | | ADSPAVNGATGHSSSLDARE |
| | | | VIPMAAVKQALREAGDEFEL |
| | | | RYRRAFSDLTSQLHITPGTA |
| | | | YQSFEQVVNELFRDGVNWGR |
| | | | IVAFFSFGGALCVESVDKEM |
| | | | QVLVSRIAAWMATYLNDHLE |
| | | | PWIQENGGWDTFVELYGNNG |
| | | | SSELIKENMHMKLYMEGTVD |
| | | | NHHFKCTSEGEGKPYEGTQT |
| | | | MRIKVVEGGPLPFAFDILAT |
| | | | SFLYGSKTFINHTQGIPDFF |
| | | | KQSFPEGFTWERVTTYEDGG |
| | | | VLTATQDTSLQDGCLIYNVK |
| | | | IRGVNFTSNGPVMQKKTLGW |
| | | | EAFTETLYPADGGLEGRNDM |
| | | | ALKLVGGSHLIANIKTTYRS |
| | | | KKPAKNLKMPGVYYVDYRLE |
| | | | RIKEANNETYVEQHEVAVAR |
| | | | YCDLPSKLGHKLN* |

### Example 8

Self-Editing mRNA Encoding Allosteric ADAR Enables Single Construct Delivery

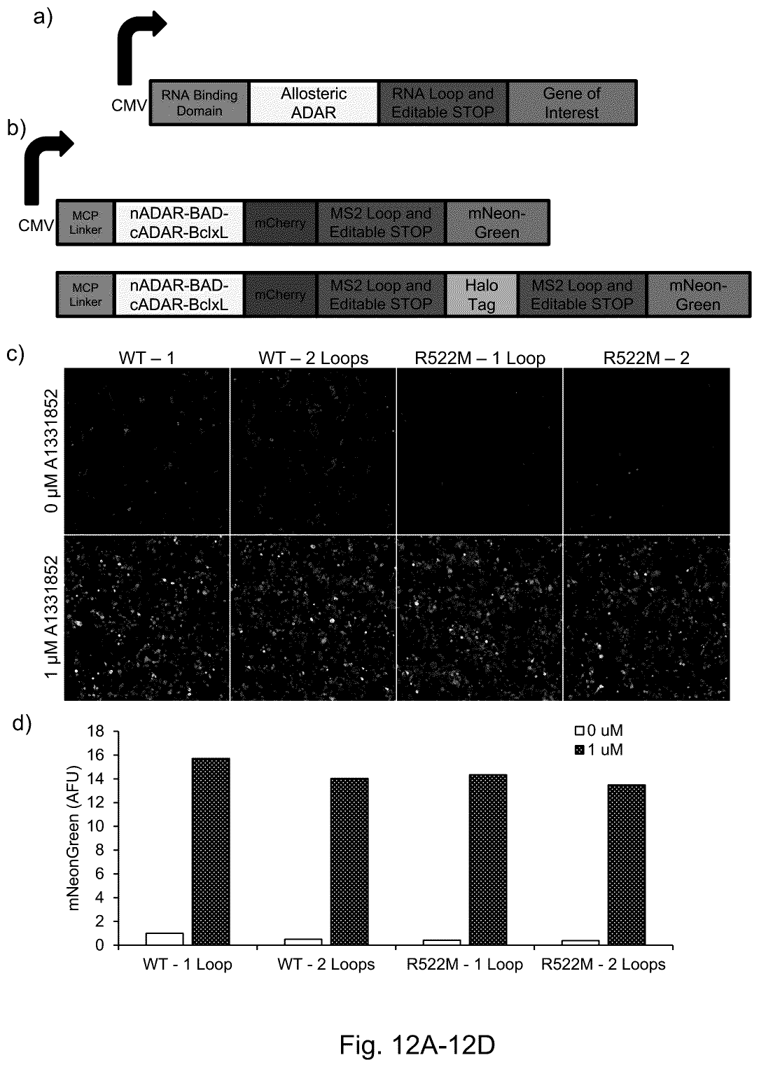

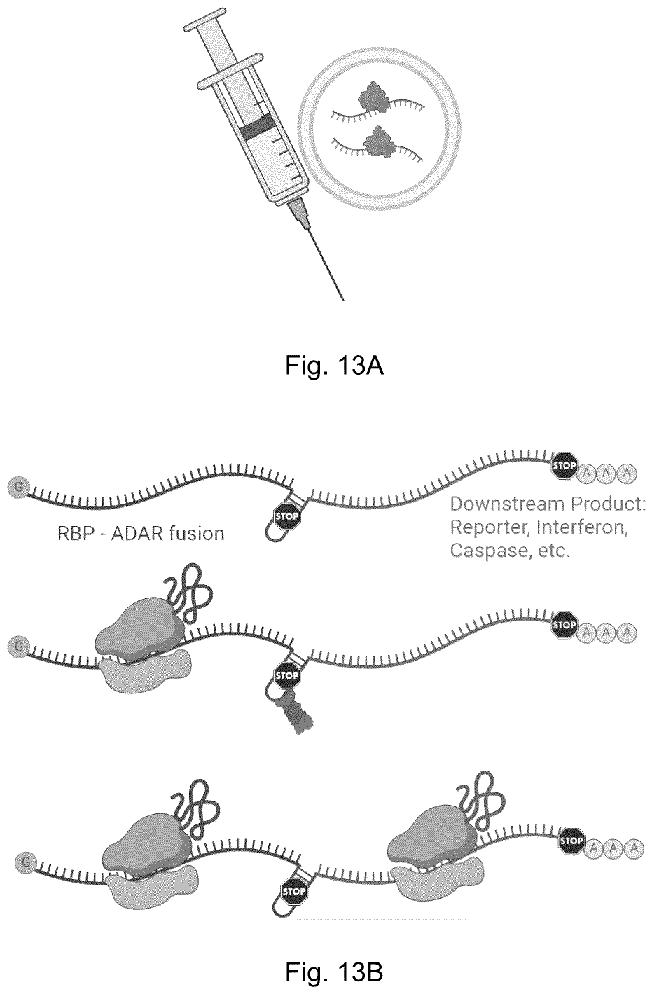

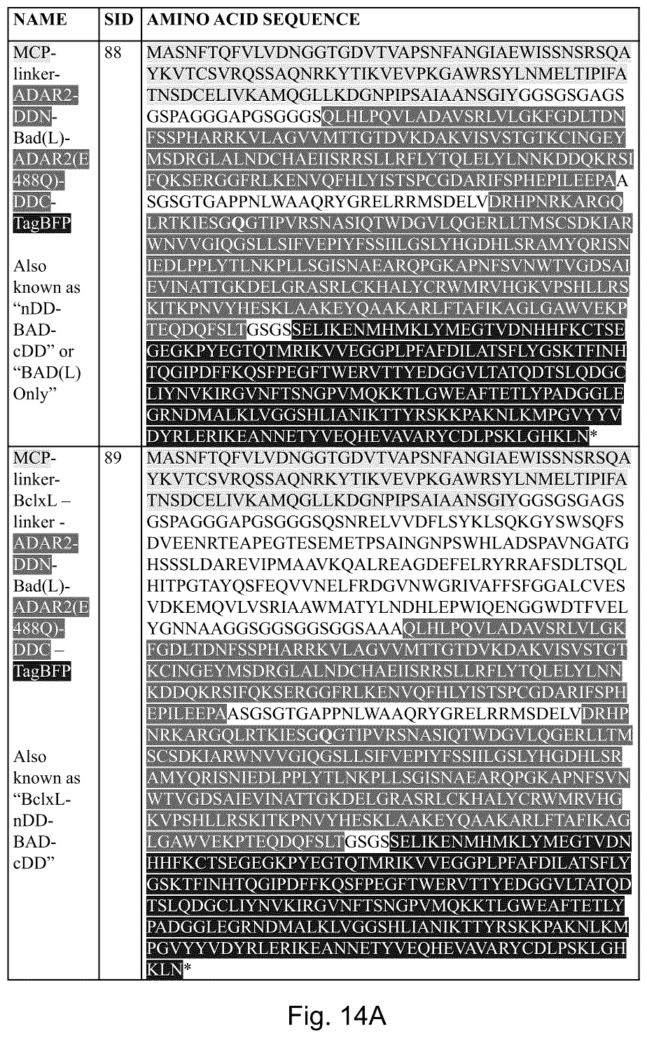

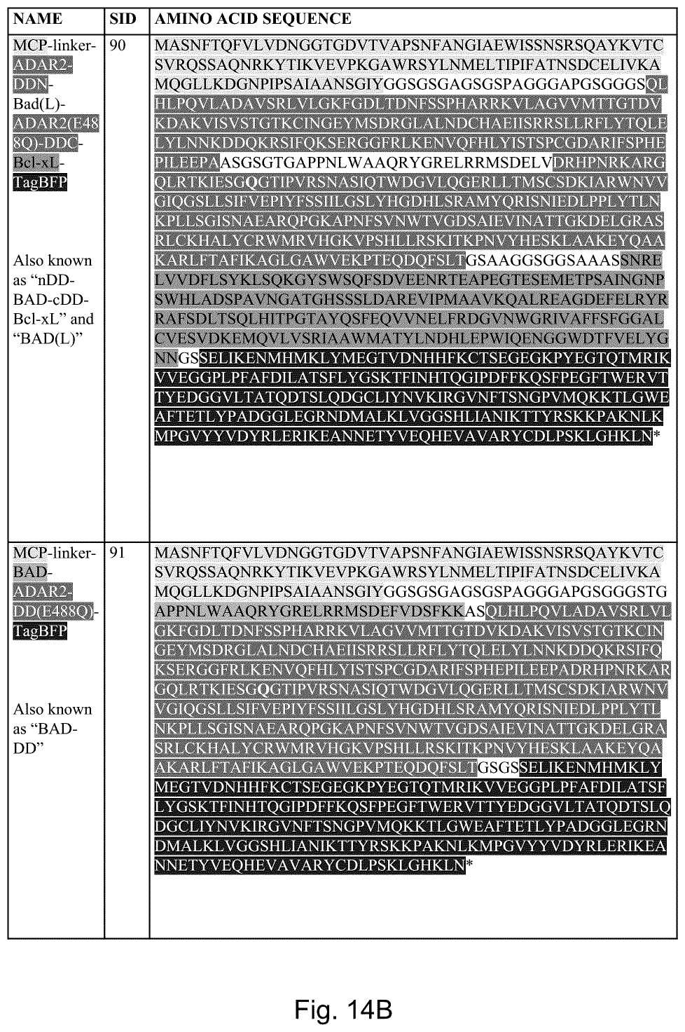

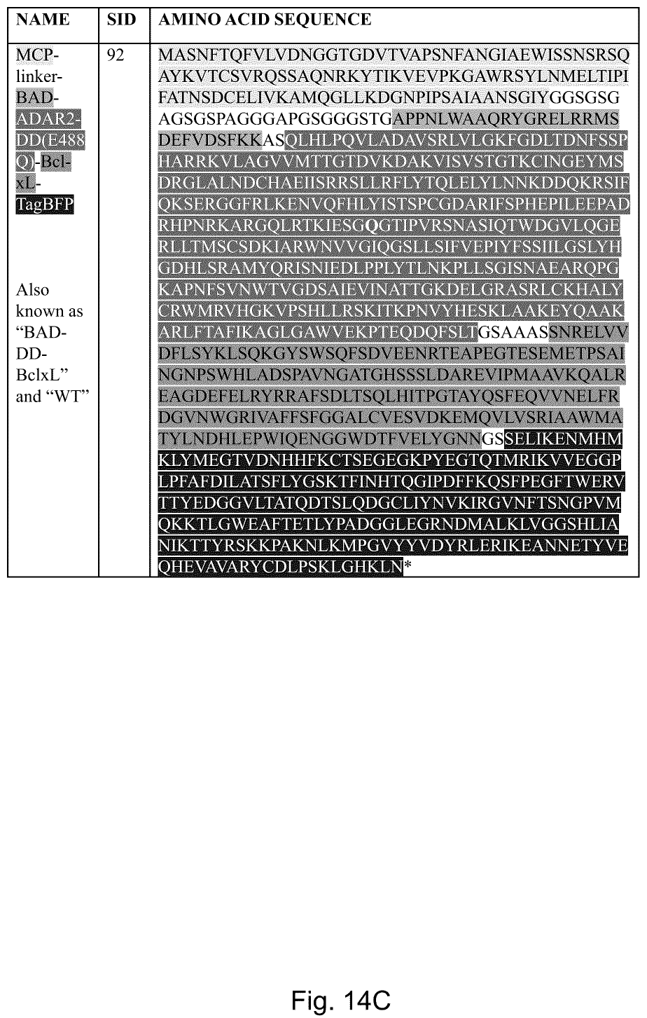

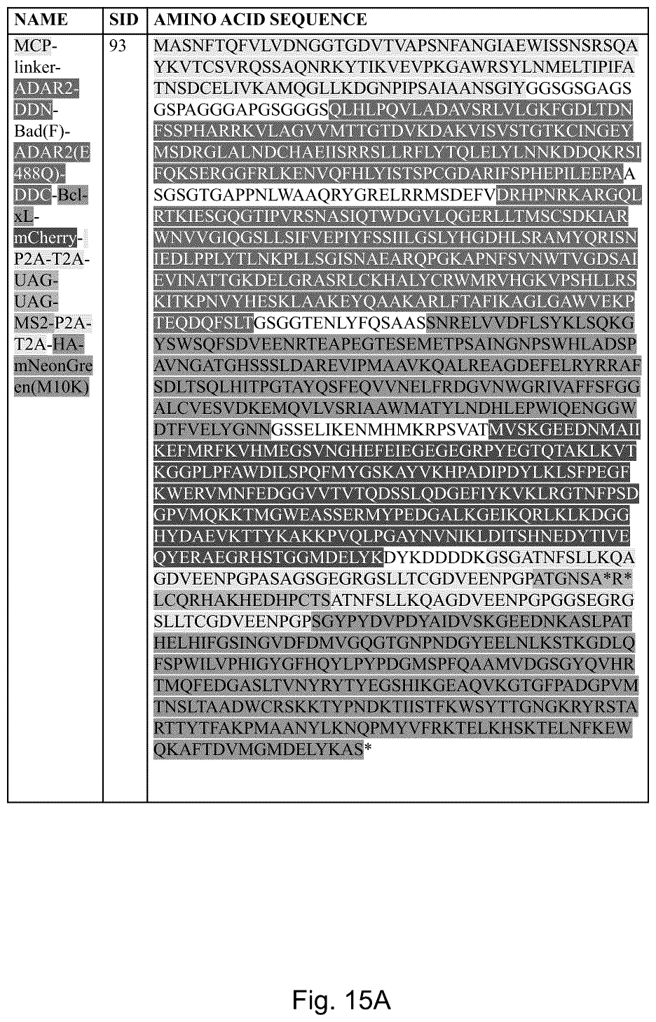

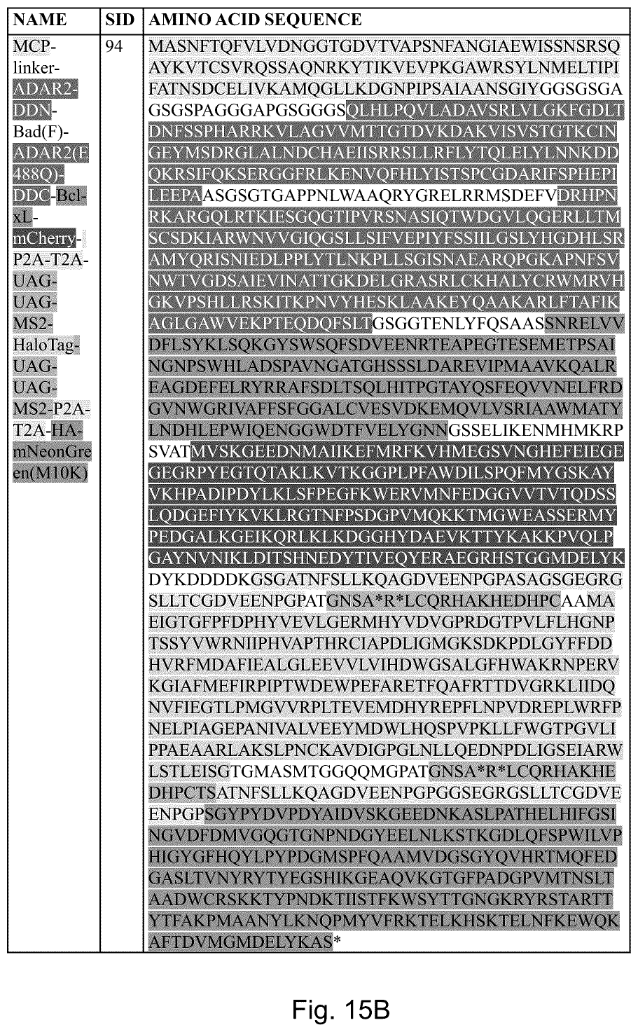

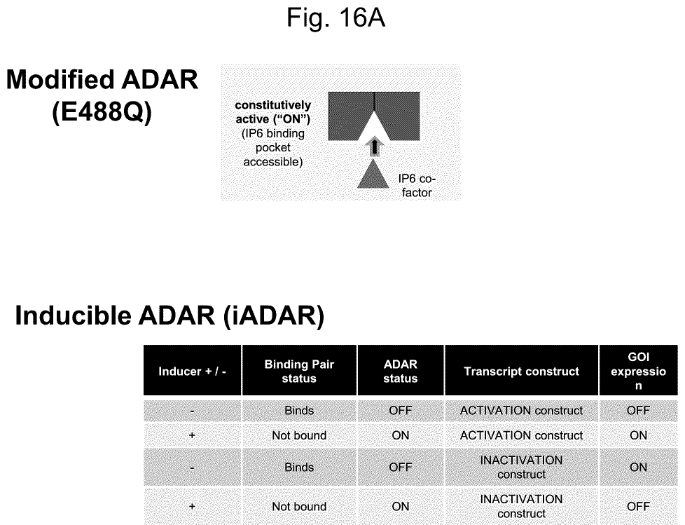

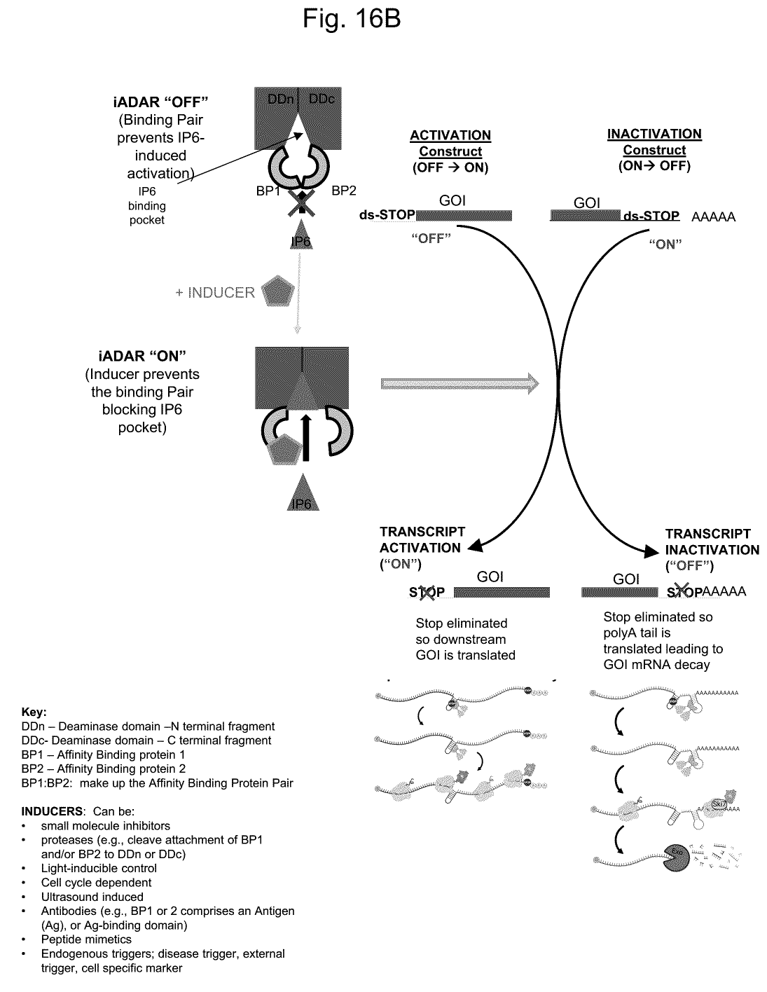

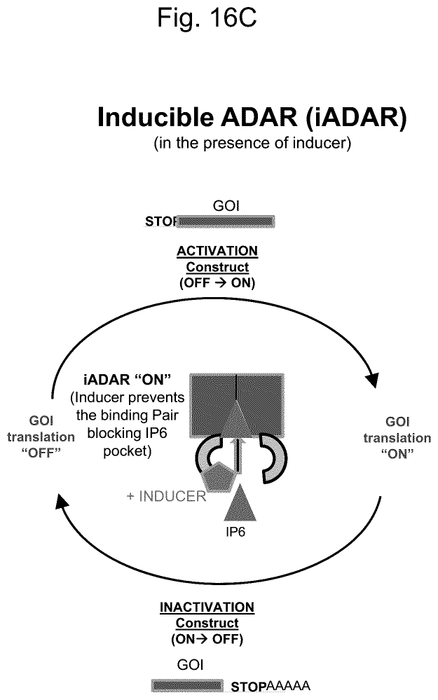

Also described herein is a single mRNA construct that encodes an upstream allosteric ADAR-DD, an editable stop codon, and a downstream gene of interest (see e.g., FIG. 12a). This configuration can be easier to deliver than two separate constructs (e.g., ADAR and mRNA transcript) and can be easier to manufacture than purifying the allosteric ADAR and delivering as an RNA-protein complex.

To test this, plasmids were created encoding an MS2 coat protein, BAD/Bcl-xL autoinhibited ADAR-DD (WT), and mCherry fusion protein upstream of either 1 or 2 editable STOP codon loops, with a downstream mNeonGreen as a readout (see e.g., FIG. 12b). HEK cells were then transfected with each of the constructs with and without 1 uM of A-1331852 and imaged the cells two days post transfection. An overlay of the mCherry (red channel) and mNeonGreen (green channel) channels shows that there is minimal translation of mNeonGreen in the absence of drug, and that all constructs lead to efficient translation of mNeonGreen with drug addition (see e.g., FIG. 12c). By quantifying the extent of mNeonGreen translation in transfected cells (see e.g., FIG. 12d), it is evident that background was reduced slightly by adding an additional editable-STOP loop (setting WT—1 Loop as 1 AFU, WT—2 Loops had background values of 0.5 AFU).

This data demonstrates the ability to administer a single, self-regulating mRNA, which can be used as a diagnostic or therapeutic.

TABLE 3
Amino Acid Sequence Table
(see e.g., FIG. 12, 15)

| | SEQ | | |
|---|---|---|---|
| | ID | See e.g., | AMINO ACID |
| NAME | NO | FIGS. | SEQUENCE |
| MCP-linker- | 93 | 12c, 12d | MASNFTQFVLVDNGG |
| ADAR2- | & | “WT 1 | TGDVTVAPSNFANGI |
| DDN- | 411 | Loop” | AEWISSNSRSQAYKV |
| Bad(F)- | | Note | TCSVRQSSAQNRKYT |
| ADAR2(E4 | | mutation | IKVEVPKGAWRSYLN |
| 88Q)-DDC- | | site | MELTIPIFATNSDCE |
| Bcl-xL- | | R522M | LIVKAMQGLLKDGNP |
| mCherry- | | is | IPSAIAANSGIYGGS |
| P2A-T2A- | | bolded | GSGAGSGSPAGGGAP |
| UAG-UAG- | | | GSGGGSQLHLPQVLA |
| MS2-P2A- | | | DAVSRLVLGKFGDLT |
| T2A-HA- | | | DNFSSPHARRKVLAG |
| mNeonGreen | | | VVMTTGTDVKDAKVI |
| (M10K) | | | SVSTGTKCINGEYMS |
| | | | DRGLALNDCHAEIIS |
| | | | RRSLLRFLYTQLELY |
| | | | LNNKDDQKRSIFQKS |
| | | | ERGGFRLKENVQFHL |
| | | | YISTSPCGDARIFSP |
| | | | HEPILEEPAASGSGT |
| | | | GAPPNLWAAQRYGRE |
| | | | LRRMSDEFVDRHPNR |
| | | | KARGQLRTKIESGQG |
| | | | TIPVRSNASIQTWDG |
| | | | VLQGERLLTMSCSDK |
| | | | IARWNVVGIQGSLLS |
| | | | IFVEPIYFSSIILGS |
| | | | LYHGDHLSRAMYQRI |
| | | | SNIEDLPPLYTLNKP |
| | | | LLSGISNAEARQPGK |
| | | | APNFSVNWTVGDSAI |
| | | | EVINATTGKDELGRA |
| | | | SRLCKHALYCRWMRV |
| | | | HGKVPSHLLRSKITK |
| | | | PNVYHESKLAAKEYQ |
| | | | AAKARLFTAFIKAGL |
| | | | GAWVEKPTEQDQFSL |
| | | | TGSGGTENLYFQSAA |
| | | | SSNRELVVDFLSYKL |
| | | | SQKGYSWSQFSDVEE |
| | | | NRTEAPEGTESEMET |
| | | | PSAINGNPSWHLADS |
| | | | PAVNGATGHSSSLDA |
| | | | REVIPMAAVKQALRE |
| | | | AGDEFELRYRRAFSD |
| | | | LTSQLHITPGTAYQS |
| | | | FEQVVNELFRDGVNW |
| | | | GRIVAFFSFGGALCV |
| | | | ESVDKEMQVLVSRIA |
| | | | AWMATYLNDHLEPWI |
| | | | QENGGWDTFVELYGN |
| | | | NGSSELIKENMHMKR |
| | | | PSVATMVSKGEEDNM |
| | | | AIIKEFMRFKVHMEG |
| | | | SVNGHEFEIEGEGEG |
| | | | RPYEGTQTAKLKVTK |
| | | | GGPLPFAWDILSPQF |
| | | | MYGSKAYVKHPADIP |
| | | | DYLKLSFPEGFKWER |
| | | | VMNFEDGGVVTVTQD |
| | | | SSLQDGEFIYKVKLR |
| | | | GTNFPSDGPVMQKKT |
| | | | MGWEASSERMYPEDG |
| | | | ALKGEIKQRLKLKDG |
| | | | GHYDAEVKTTYKAKK |
| | | | PVQLPGAYNVNIKLD |
| | | | ITSHNEDYTIVEQYE |
| | | | RAEGRHSTGGMDELY |
| | | | KDYKDDDDKGSGATN |
| | | | FSLLKQAGDVEENPG |
| | | | PASAGSGEGRGSLLT |
| | | | CGDVEENPGPATGNS |
| | | | A*R*LCQRHAKHEDH |
| | | | PCTSATNFSLLKQAG |
| | | | DVEENPGPGGSEGRG |
| | | | SLLTCGDVEENPGPS |
| | | | GYPYDVPDYAIDVSK |
| | | | GEEDNKASLPATHEL |
| | | | HIFGSINGVDFDMVG |
| | | | QGTGNPNDGYEELNL |
| | | | KSTKGDLQFSPWILV |
| | | | PHIGYGFHQYLPYPD |
| | | | GMSPFQAAMVDGSGY |
| | | | QVHRTMQFEDGASLT |
| | | | VNYRYTYEGSHIKGE |
| | | | AQVKGTGFPADGPVM |
| | | | TNSLTAADWCRSKKT |
| | | | YPNDKTIISTFKWSY |
| | | | TTGNGKRYRSTARTT |
| | | | YTFAKPMAANYLKNQ |
| | | | PMYVFRKTELKHSKT |
| | | | ELNFKEWQKAFTDVM |
| | | | GMDELYKAS* |
| MCP-linker- | 94, | 12c, 12d | MASNFTQFVLVDNGG |
| ADAR2- | 393, | “WT-2 | TGDVTVAPSNFANGI |
| DDN- | and | Loop” | AEWISSNSRSQAYKV |
| Bad(F)- | 411 | Note | TCSVRQSSAQNRKYT |
| ADAR2(E4 | | mutation | IKVEVPKGAWRSYLN |
| 88Q)-DDC- | | site | MELTIPIFATNSDCE |
| Bcl-xL- | | R522M | LIVKAMQGLLKDGNP |
| mCherry- | | is | IPSAIAANSGIYGGS |
| P2A-T2A- | | bolded | GSGAGSGSPAGGGAP |
| UAG-UAG- | | | GSGGGSQLHLPQVLA |
| MS2- | | | DAVSRLVLGKFGDLT |
| Halo Tag- | | | DNFSSPHARRKVLAG |
| UAG-UAG- | | | VVMTTGTDVKDAKVI |
| MS2-P2A- | | | SVSTGTKCINGEYMS |
| T2A-HA- | | | DRGLALNDCHAEIIS |
| mNeonGreen | | | RRSLLRFLYTQLELY |
| (M10K) | | | LNNKDDQKRSIFQKS |
| | | | ERGGFRLKENVQFHL |
| | | | YISTSPCGDARIFSP |
| | | | HEPILEEPAASGSGT |
| | | | GAPPNLWAAQRYGRE |
| | | | LRRMSDEFVDRHPNR |
| | | | KARGQLRTKIESGQG |
| | | | TIPVRSNASIQTWDG |
| | | | VLQGERLLTMSCSDK |
| | | | IARWNVVGIQGSLLS |
| | | | IFVEPIYFSSIILGS |
| | | | LYHGDHLSRAMYQRI |
| | | | SNIEDLPPLYTLNKP |
| | | | LLSGISNAEARQPGK |
| | | | APNFSVNWTVGDSAI |
| | | | EVINATTGKDELGRA |
| | | | SRLCKHALYCRWMRV |
| | | | HGKVPSHLLRSKITK |
| | | | PNVYHESKLAAKEYQ |
| | | | AAKARLFTAFIKAGL |
| | | | GAWVEKPTEQDQFSL |
| | | | TGSGGTENLYFQSAA |
| | | | SSNRELVVDFLSYKL |
| | | | SQKGYSWSQFSDVEE |
| | | | NRTEAPEGTESEMET |
| | | | PSAINGNPSWHLADS |
| | | | PAVNGATGHSSSLDA |
| | | | REVIPMA |

Below are DNA sequences of interest, e.g., for Table 3.

SEQ ID NO: 402, MCP-linker-ADAR2-DDN-
Bad(F)-ADAR2(E488Q)-DDC-Bcl-XL-
mCherry-P2A-T2A-UAG-UAG-MS2-P2A-T2A-HA-
mNeonGreen(M10K), see e.g., polypeptide
in SEQ ID NO: 93; UAG-UAG-MS2 domain is
bold-italicized
CTTATCGAAATTAATACGACTCACTATAGGGAGACCCAAGCTGGc
tagaggatcgaacccttaaggccaccatggcgtccaatttcactc
agtttgtgctggttgacaacggcgggaccggggacgttacggtag
ccccctcaaactttgccaacggtatagcggagtggataagcagca
attctaggagtcaagcatacaaagttacatgcagcgtgcgccaat
ctagcgctcagaatcgcaagtacaccattaaagtagaggtcccca
agggagcctggagaagctatcttaacatggagttgaccataccaa
tcttcgctaccaactctgactgtgaactcattgtgaaagccatgc
aaggtctgctcaaggatggtaacccaattccgtccgctatcgctg
ccaactctgggatttacgggggcagtgggagcggtgcaggatctg
gtagtccagctgggggaggagcaccgggtagcggtggggggtctc
agctgcacctgccccaggttctcgcagacgccgtatcccgccttg
tactgggcaagtttggtgatcttactgacaatttttcatctcctc
atgcgaggcggaaagtactcgcaggcgtcgtcatgacgaccggaa
ctgacgtgaaagacgccaaagtcatctctgtctccacgggcacaa
agtgcataaacggggagtacatgagcgaccgggggctggcactga
atgattgtcacgctgaaataatatctaggcgatctctgcttagat
ttctctacactcaactcgaattgtaccttaacaacaaagatgacc
agaaacgcagtatatttcagaaatcagaacgcggcggatttcgac
ttaaggaaaacgttcagttccacttgtatatcagcacatcccctt
gcggtgacgcccgaatcttttccccgcacgagccgatattggagg
agcccgcgGCTAGCGGGTCGGGCACCGGTGCTCCACCCAATCTCT
GGGCAGCGCAGCGCTACGGCCGTGAGCTCAGAAGGATGTCCGATG
AGttcGTCGACagacatcctaataggaaggctagaggccaacttc
ggacgaagattgaaagtggccagggtactatcccggtgcggtcca
acgctagtattcaaacgtgggacggagtccttcaaggtgaacggc
tgttgacaatgagctgctcagacaaaatcgcgcgctggaatgtag
tgggaatccaaggcagcctcttgagcatattcgtagaacccatat
atttctcatccattattttgggctctctgtatcatggtgaccatc
tgtcaagggctatgtaccaacgaatttctaatatcgaggatcttc
ctccactctatacactcaataagcctctcttgtccgggatatcaa
acgctgaggcccgccagccagggaaagctcctaacttcagtgtta
actggaccgttggtgattctgcgatagaggtcatcaacgccacga
caggtaaggatgagctcggtagagcctcacgcctgtgtaaacacg
cgttgtattgtagatggatgagagtacatgggaaggtcccatctc
acttgctccgaagcaagatcactaagcctaatgtgtatcatgagt
caaaactcgcggctaaagaataccaggcagccaaagctcgacttt
ttacagcttttattaaggcagggctcggggcatgggtcgagaagc
cgaccgagcaggaccaattctctctgacggggagcGGAGGTACGG
AGAATTTGTATTTTCAGAGCGCCGCTTCAAGTAACCGGGAGCTGG
TGGTTGACTTTCTCTCCTACAAGCTTTCCCAGAAAGGATACAGCT
GGAGTCAGTTTAGTGATGTGGAAGAGAACAGGACTGAGGCCCCAG
AAGGGACTGAATCGGAGATGGAGACCCCCAGTGCCATCAATGGCA
ACCCATCCTGGCACCTGGCAGACAGCCCCGCGGTGAATGGAGCCA
CTGGCCACAGCAGCAGTTTGGATGCCCGGGAGGTGATCCCCATGG
CAGCAGTAAAGCAAGCGCTGAGGGAGGCAGGCGACGAGTTTGAAC
TGCGGTACCGGCGGGCATTCAGTGACCTGACATCCCAGCTCCACA
TCACCCCAGGGACAGCATATCAGAGCTTTGAACAGGTAGTGAATG
AACTCTTCCGGGATGGGGTAAACTGGGGTCGCATTGTGGCCTTTT
TCTCCTTCGGCGGGGCACTGTGCGTGGAAAGCGTAGACAAGGAGA
TGCAGGTATTGGTGAGTCGGATCGCAGCTTGGATGGCCACTTACC
TGAATGACCACCTAGAGCCTTGGATCCAGGAGAACGGCGGCTGGG
ATACTTTTGTGGAACTCTATGGGAACAATggatccAGCGAGCTGA
TTAAGGAGAACATGCACATGAAGCGCCCatcggtcgccaccatgg
tgagcaagggcgaggaggataacatggccatcatcaaggagttca
tgcgcttcaaggtgcacatggagggctccgtgaacggccacgagt
tcgagatcgagggcgagggcgagggccgcccctacgagggcaccc
agaccgccaagctgaaggtgaccaagggtggccccctgcccttcg
cctgggacatcctgtcccctcagttcatgtacggctccaaggcct
acgtgaagcaccccgccgacatccccgactacttgaagctgtcct
tccccgagggcttcaagtgggagcgcgtgatgaacttcgaggacg
gcggcgtggtgaccgtgacccaggactcctccctgcaggacggcg
agttcatctacaaggtgaagctgcgcggcaccaacttcccctccg
acggccccgtaatgcagaagaagaccatgggctgggaggcctcct
ccgagcggatgtaccccgaggacggcgccctgaagggcgagatca
agcagaggctgaagctgaaggacggcggccactacgacgctgagg
tcaagaccacctacaaggccaagaagcccgtgcagctgcccggcg
cctacaacgtcaacatcaagttggacatcacctcccacaacgagg
actacaccatcgtggaacagtacgaacgcgccgagggccgccact
ccaccggcggcatggacgagctgTACaaggattacaaggatgacg
atgacaaaGGTAGCGGGGCAACTAATTTTAGCTTACTCAAACAGG
CTGGGGACGTCGAGGAGAATCCAGGCCCTGCATCCGCTGGCTCTG
GAGAAGGACGAGGCTCCTTGCTCACCTGTGGAGATGTCGAAGAGA
ACCCAGGTCCTGCAACCGGGAATTCCGCGTAGCGCTAGCTTTGCC
AGCGCCACGCGaaACATGAGGATcACCCATGTACTAGTGCCACAA
ACTTCTCTCTGCTAAAGCAAGCAGGTGATGTTGAAGAAAACCCAG
GGCCTGGAGGGTCCGAGGGCAGGGGAAGTCTCCTAACATGCGGGG
ACGTGGAGGAAAATCCCGGCCCATCCGGATATCCCTACGATGTGC
CCGATTACGCTATCGATgtgagcaagggcgaAgaAgataacaAgg
cctctctcccagcgacacatgagttacacatctttggctccatca
acggtgtggactttgacatggtgggtcagggcaccggcaatccaa
atgatggttatgaggagttaaacctgaagtccaccaagggtgacc
tccagttctccccctggattctggtccctcatatcgggtatggct
tccatcagtacctgccctaccctgacgggatgtcgcctttccagg
ccgccatggtagatggcAGCggataccaagtccatcgcacaatgc
agtttgaagatggtgcctcccttactgttaactaccgctacacct
acgagggaagccacatcaaaggagaggcccaggtgaaggggactg
gtttccctgctgacggtcctgtgatgaccaactcgctgaccgctg
cggactggtgcaggtcgaagaagacttaccccaacgacaaaacca
tcatcagtacctttaagtggagttacaccactggaaatggcaagA
GAtaccggagcactgcgcggaccacctacacctttgccaagccaa
tggcggctaactatctgaagaaccagccgatgtacgtgttccgta
agacggagctcaagcactccaagaccgagctcaacttcaaggagt
ggcaaaaggcctttaccgatgtgatgGGAatggacGAGCTGTATa
agGCTAGCTAAGCGGCCGCTCGAGTCTAGAGGGCCCGCGGTTCGA
AGGTAAGCCTATCCCTAACCCTCTCCTCGGTCTCGATTCTACGCG
TACCGGTCATCATCACCATCACCATTGAGTTTAAACCCGCTGATC
AG
SEQ ID NO: 99, MCP-linker-ADAR2-DDN-Bad(F)-
ADAR2(E488Q)-DDC-Bcl-xL-mCherry-P2A-T2A-UAG-
UAG-MS2-HaloTag-UAG-UAG-MS2-P2A-T2A-HA-
mNeonGreen(M10K);
see e.g., polypeptide in SEQ ID NO: 94;
UAG-UAG-MS2 domains are bold-italicized
CTTATCGAAATTAATACGACTCACTATAGGGAGACCCAAGCTGGc
tagaggatcgaacccttaaggccaccatggcgtccaatttcactc
agtttgtgctggttgacaacggcgggaccggggacgttacggtag
ccccctcaaactttgccaacggtatagcggagtggataagcagca
attctaggagtcaagcatacaaagttacatgcagcgtgcgccaat
ctagcgctcagaatcgcaagtacaccattaaagtagaggtcccca
agggagcctggagaagctatcttaacatggagttgaccataccaa
tcttcgctaccaactctgactgtgaactcattgtgaaagccatgc
aaggtctgctcaaggatggtaacccaattccgtccgctatcgctg
ccaactctgggatttacgggggcagtgggagcggtgcaggatctg
gtagtccagctgggggaggagcaccgggtagcggtggggggtctc
agctgcacctgccccaggttctcgcagacgccgtatcccgccttg
tactgggcaagtttggtgatcttactgacaatttttcatctcctc
atgcgaggcggaaagtactcgcaggcgtcgtcatgacgaccggaa
ctgacgtgaaagacgccaaagtcatctctgtctccacgggcacaa
agtgcataaacggggagtacatgagcgaccgggggctggcactga
atgattgtcacgctgaaataatatctaggcgatctctgcttagat
ttctctacactcaactcgaattgtaccttaacaacaaagatgacc
agaaacgcagtatatttcagaaatcagaacgcggcggatttcgac
ttaaggaaaacgttcagttccacttgtatatcagcacatcccctt
gcggtgacgcccgaatcttttccccgcacgagccgatattggagg
agcccgcgGCTAGCGGGTCGGGCACCGGTGCTCCACCCAATCTCT
GGGCAGCGCAGCGCTACGGCCGTGAGCTCAGAAGGATGTCCGATG
AGttcGTCGACagacatcctaataggaaggctagaggccaacttc
ggacgaagattgaaagtggccagggtactatcccggtgcggtcca
acgctagtattcaaacgtgggacggagtccttcaaggtgaacggc
tgttgacaatgagctgctcagacaaaatcgcgcgctggaatgtag
tgggaatccaaggcagcctcttgagcatattcgtagaacccatat
atttctcatccattattttgggctctctgtatcatggtgaccatc
tgtcaagggctatgtaccaacgaatttctaatatcgaggatcttc
ctccactctatacactcaataagcctctcttgtccgggatatcaa
acgctgaggcccgccagccagggaaagctcctaacttcagtgtta
actggaccgttggtgattctgcgatagaggtcatcaacgccacga
caggtaaggatgagctcggtagagcctcacgcctgtgtaaacacg
cgttgtattgtagatggatgagagtacatgggaaggtcccatctc
acttgctccgaagcaagatcactaagcctaatgtgtatcatgagt
caaaactcgcggctaaagaataccaggcagccaaagctcgacttt
ttacagcttttattaaggcagggctcggggcatgggtcgagaagc
cgaccgagcaggaccaattctctctgacggggagcGGAGGTACGG
AGAATTTGTATTTTCAGAGCGCCGCTTCAAGTAACCGGGAGCTGG
TGGTTGACTTTCTCTCCTACAAGCTTTCCCAGAAAGGATACAGCT
GGAGTCAGTTTAGTGATGTGGAAGAGAACAGGACTGAGGCCCCAG
AAGGGACTGAATCGGAGATGGAGACCCCCAGTGCCATCAATGGCA
ACCCATCCTGGCACCTGGCAGACAGCCCCGCGGTGAATGGAGCCA
CTGGCCACAGCAGCAGTTTGGATGCCCGGGAGGTGATCCCCATGG
CAGCAGTAAAGCAAGCGCTGAGGGAGGCAGGCGACGAGTTTGAAC
TGCGGTACCGGCGGGCATTCAGTGACCTGACATCCCAGCTCCACA
TCACCCCAGGGACAGCATATCAGAGCTTTGAACAGGTAGTGAATG
AACTCTTCCGGGATGGGGTAAACTGGGGTCGCATTGTGGCCTTTT
TCTCCTTCGGCGGGGCACTGTGCGTGGAAAGCGTAGACAAGGAGA
TGCAGGTATTGGTGAGTCGGATCGCAGCTTGGATGGCCACTTACC
TGAATGACCACCTAGAGCCTTGGATCCAGGAGAACGGCGGCTGGG
ATACTTTTGTGGAACTCTATGGGAACAATggatccAGCGAGCTGA
TTAAGGAGAACATGCACATGAAGCGCCCatcggtcgccaccatgg
tgagcaagggcgaggaggataacatggccatcatcaaggagttca
tgcgcttcaaggtgcacatggagggctccgtgaacggccacgagt
tcgagatcgagggcgagggcgagggccgcccctacgagggcaccc
agaccgccaagctgaaggtgaccaagggtggccccctgcccttcg
cctgggacatcctgtcccctcagttcatgtacggctccaaggcct
acgtgaagcaccccgccgacatccccgactacttgaagctgtcct
tccccgagggcttcaagtgggagcgcgtgatgaacttcgaggacg
gcggcgtggtgaccgtgacccaggactcctccctgcaggacggcg
agttcatctacaaggtgaagctgcgcggcaccaacttcccctccg
acggccccgtaatgcagaagaagaccatgggctgggaggcctcct
ccgagcggatgtaccccgaggacggcgccctgaagggcgagatca
agcagaggctgaagctgaaggacggcggccactacgacgctgagg
tcaagaccacctacaaggccaagaagcccgtgcagctgcccggcg
cctacaacgtcaacatcaagttggacatcacctcccacaacgagg
actacaccatcgtggaacagtacgaacgcgccgagggccgccact
ccaccggcggcatggacgagctgTACaaggattacaaggatgacg
atgacaaaGGTAGCGGGGCAACTAATTTTAGCTTACTCAAACAGG
CTGGGGACGTCGAGGAGAATCCAGGCCCTGCATCCGCTGGCTCTG
GAGAAGGACGAGGCTCCTTGCTCACCTGTGGAGATGTCGAAGAGA
ACCCAGGTCCTGCAACCGGGAATTCCGCGTAGCGCTAGCTTTGCC
AGCGCCACGCGaaACATGAGGATcACCCATGTGCCGCTATGGCAG
AAATCGGTACTGGCTTTCCATTCGACCCCCATTATGTGGAAGTCC
TGGGCGAGCGCATGCACTACGTCGATGTTGGTCCGCGCGATGGCA
CCCCTGTGCTGTTCCTGCACGGTAACCCGACCTCCTCCTACGTGT
GGCGCAACATCATCCCGCATGTTGCACCGACCCATCGCTGCATTG
CTCCAGACCTGATCGGTATGGGCAAATCCGACAAACCAGACCTGG
GTTATTTCTTCGACGACCACGTCCGCTTCATGGATGCCTTCATCG
AAGCCCTGGGTCTGGAAGAGGTCGTCCTGGTCATTCACGACTGGG
GCTCCGCTCTGGGTTTCCACTGGGCCAAGCGCAATCCAGAGCGCG
TCAAAGGTATTGCATTTATGGAGTTCATCCGCCCTATCCCGACCT
GGGACGAATGGCCAGAATTTGCCCGCGAGACCTTCCAGGCCTTCC
GCACCACCGACGTCGGCCGCAAGCTGATCATCGATCAGAACGTTT
TTATCGAGGGTACGCTGCCGATGGGTGTCGTCCGCCCGCTGACTG
AAGTCGAGATGGACCATTACCGCGAGCCGTTCCTGAATCCTGTTG
ACCGCGAGCCACTGTGGCGCTTCCCAAACGAGCTGCCAATCGCCG
GTGAGCCAGCGAACATCGTCGCGCTGGTCGAAGAATACATGGACT
GGCTGCACCAGTCCCCTGTCCCGAAGCTGCTGTTCTGGGGCACCC
CAGGCGTTCTGATCCCACCGGCCGAAGCCGCTCGCCTGGCCAAAA
GCCTGCCTAACTGCAAGGCTGTGGACATCGGCCCGGGTCTGAATC
TGCTGCAAGAAGACAACCCGGACCTGATCGGCAGCGAGATCGCGC
GCTGGCTGTCGACGCTCGAGATTTCTGGCACCGGTATGGCATCTA
TGACTGGAGGCCAACAGATGgGTCCTGCAACCGGGAATTCCGCGT
AGCGCTAGCTTTGCCAGCGCCACGCGaaACATGAGGATcACCCAT
GTACTAGTGCCACAAACTTCTCTCTGCTAAAGCAAGCAGGTGATG
TTGAAGAAAACCCAGGGCCTGGAGGGTCCGAGGGCAGGGGAAGTC
TCCTAACATGCGGGGACGTGGAGGAAAATCCCGGCCCATCCGGAT
ATCCCTACGATGTGCCCGATTACGCTATCGATgtgagcaagggcg
aAgaAgataacaAggcctctctcccagcgacacatgagttacaca
tctttggctccatcaacggtgtggactttgacatggtgggtcagg
gcaccggcaatccaaatgatggttatgaggagttaaacctgaagt
ccaccaagggtgacctccagttctccccctggattctggtccctc
atatcgggtatggcttccatcagtacctgccctaccctgacggga
tgtcgcctttccaggccgccatggtagatggcAGCggataccaag
tccatcgcacaatgcagtttgaagatggtgcctcccttactgtta
actaccgctacacctacgagggaagccacatcaaaggagaggccc
aggtgaaggggactggtttccctgctgacggtcctgtgatgacca
actcgctgaccgctgcggactggtgcaggtcgaagaagacttacc
ccaacgacaaaaccatcatcagtacctttaagtggagttacacca
ctggaaatggcaagAGAtaccggagcactgcgcggaccacctaca
cctttgccaagccaatggggctaactatctgaagaaccagccgat
gtacgtgttccgtaagacggagctcaagcactccaagaccgagct
caacttcaaggagtggcaaaaggcctttaccgatgtgatgGGAat
ggacGAGCTGTATaagGCTAGCTAAGCGGCCGCTCGAGTCTAGAG
GGCCCGCGGTTCGAAGGTAAGCCTATCCCTAACCCTCTCCTCGGT
CTCGATTCTACGCGTACCGGTCATCATCACCATCACCATTGAGTT
TAAACCCGCTGATCAG

### Example 9

### Development of Grazoprevir Activated ADAR

Described in other examples is a drug responsivity (small molecule) based upon the heterodimerization of Bcl-2 family proteins and BH3 peptides (BclxL & Mel-1 along with Bad, Bid, & MS1 peptides). Described in Example 9 is a system based upon the drug induced dissociation of an antiviral peptide with a viral protease (e.g., CP5-46-4D5E & HCV protease NS3 from genotype 1B). This peptide binds with high affinity to the HCV protease, and can be dissociated by adding protease inhibitors like grazoprevir. Furthermore, there are no mutations and the protein utilizes the insertion site).

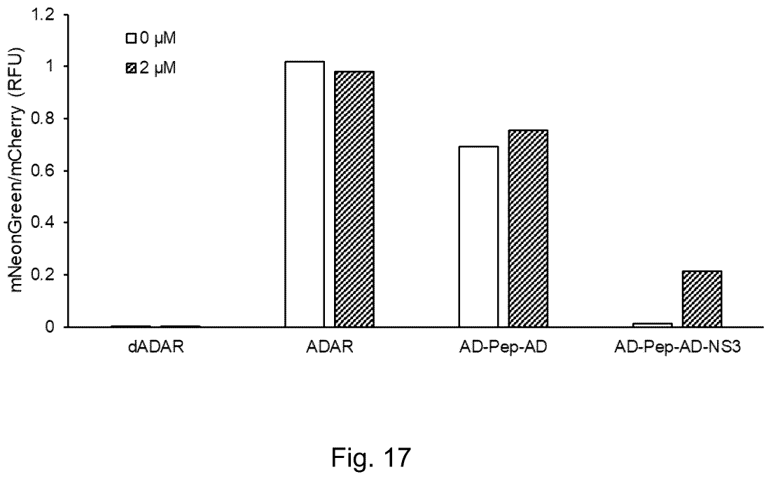

This drug-dissociative pair was utilized to engineer a NS3-inhibitor activated ADAR by fusing the peptide in the 5′ binding loop and the NS3 protease to the C-terminus of ADAR2-DD. In some embodiments, a monomeric, tandem-dimer form of MS2 coat protein (tdMCP) can be used. Inserting the CP5-46-4D5E peptide into ADAR2-DD does not greatly influence editing activity, but fusing NS3 to the C-terminus leads to a drug-dependent reduction in editing efficiency (˜18× higher mNG/mCh with grazoprevir) (see e.g., FIG. 17: HEK cells, transiently transfected with 2 μM grazoprevir added at the time of transfection, imaged 48 hours later, data processed by IMAGEJ to get the median value of the ratio of mNeonGreen to mCherry, gated by BFP for transfection).

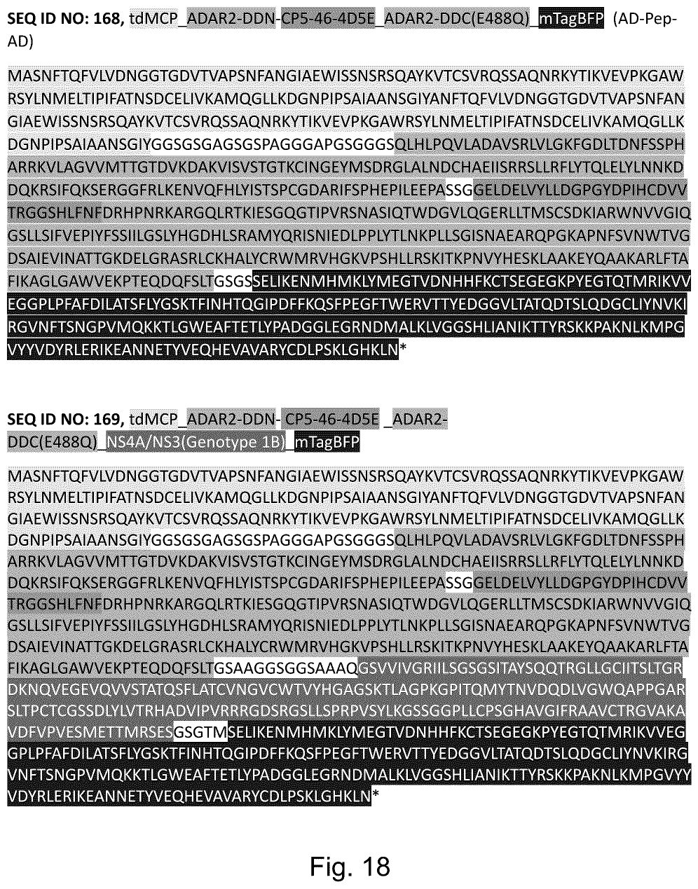

SEQ ID NO: 168, tdMCP_ADAR2-DDN-CP5-46-
4D5E_ADAR2-DDC(E488Q)_mTagBFP
(AD-Pep-AD) (see e.g., FIG. 18)
MASNFTQFVLVDNGGTGDVTVAPSNFANGIAEWISSNSRSQAYKV
TCSVRQSSAQNRKYTIKVEVPKGAWRSYLNMELTIPIFATNSDCE
LIVKAMQGLLKDGNPIPSAIAANSGIYANFTQFVLVDNGGTGDVT
VAPSNFANGIAEWISSNSRSQAYKVTCSVRQSSAQNRKYTIKVEV
PKGAWRSYLNMELTIPIFATNSDCELIVKAMQGLLKDGNPIPSAI
AANSGIYGGSGSGAGSGSPAGGGAPGSGGGSQLHLPQVLADAVSR
LVLGKFGDLTDNFSSPHARRKVLAGVVMTTGTDVKDAKVISVSTG
TKCINGEYMSDRGLALNDCHAEIISRRSLLRFLYTQLELYLNNKD
DQKRSIFQKSERGGFRLKENVQFHLYISTSPCGDARIFSPHEPIL
EEPASSGGELDELVYLLDGPGYDPIHCDVVTRGGSHLFNFDRHPN
RKARGQLRTKIESGQGTIPVRSNASIQTWDGVLQGERLLTMSCSD
KIARWNVVGIQGSLLSIFVEPIYFSSIILGSLYHGDHLSRAMYQR
ISNIEDLPPLYTLNKPLLSGISNAEARQPGKAPNFSVNWTVGDSA
IEVINATTGKDELGRASRLCKHALYCRWMRVHGKVPSHLLRSKIT
KPNVYHESKLAAKEYQAAKARLFTAFIKAGLGAWVEKPTEQDQFS
LTGSGSSELIKENMHMKLYMEGTVDNHHFKCTSEGEGKPYEGTQT
MRIKVVEGGPLPFAFDILATSFLYGSKTFINHTQGIPDFFKQSFP
EGFTWERVTTYEDGGVLTATQDTSLQDGCLIYNVKIRGVNFTSNG
PVMQKKTLGWEAFTETLYPADGGLEGRNDMALKLVGGSHLIANIK
TTYRSKKPAKNLKMPGVYYVDYRLERIKEANNETYVEQHEVAVAR
YCDLPSKLGHKLN*
SEQ ID NO: 169, tdMCP_ADAR2-
DDN-CP5-46-4D5E ADAR2-
DDC(E488Q)_NS4A/NS3(Genotype 1B)_
mTagBFP (see e.g., FIG. 18)
MASNFTQFVLVDNGGTGDVTVAPSNFANGIAEWISSNSRSQAYKV
TCSVRQSSAQNRKYTIKVEVPKGAWRSYLNMELTIPIFATNSDCE
LIVKAMQGLLKDGNPIPSAIAANSGIYANFTQFVLVDNGGTGDVT
VAPSNFANGIAEWISSNSRSQAYKVTCSVRQSSAQNRKYTIKVEV
PKGAWRSYLNMELTIPIFATNSDCELIVKAMQGLLKDGNPIPSAI
AANSGIYGGSGSGAGSGSPAGGGAPGSGGGSQLHLPQVLADAVSR
LVLGKFGDLTDNFSSPHARRKVLAGVVMTTGTDVKDAKVISVSTG
TKCINGEYMSDRGLALNDCHAEIISRRSLLRFLYTQLELYLNNKD
DQKRSIFQKSERGGFRLKENVQFHLYISTSPCGDARIFSPHEPIL
EEPASSGGELDELVYLLDGPGYDPIHCDVVTRGGSHLFNFDRHPN
RKARGQLRTKIESGQGTIPVRSNASIQTWDGVLQGERLLTMSCSD
KIARWNVVGIQGSLLSIFVEPIYFSSIILGSLYHGDHLSRAMYQR
ISNIEDLPPLYTLNKPLLSGISNAEARQPGKAPNFSVNWTVGDSA
IEVINATTGKDELGRASRLCKHALYCRWMRVHGKVPSHLLRSKIT
KPNVYHESKLAAKEYQAAKARLFTAFIKAGLGAWVEKPTEQDQFS
LTGSAAGGSGGSAAAQGSVVIVGRIILSGSGSITAYSQQTRGLLG
CIITSLTGRDKNQVEGEVQVVSTATQSFLATCVNGVCWTVYHGAG
SKTLAGPKGPITQMYTNVDQDLVGWQAPPGARSLTPCTCGSSDLY
LVTRHADVIPVRRRGDSRGSLLSPRPVSYLKGSSGGPLLCPSGHA
VGIFRAAVCTRGVAKAVDFVPVESMETTMRSESGSGTMSELIKEN
MHMKLYMEGTVDNHHFKCTSEGEGKPYEGTQTMRIKVVEGGPLPF
AFDILATSFLYGSKTFINHTQGIPDFFKQSFPEGFTWERVTTYED
GGVLTATQDTSLQDGCLIYNVKIRGVNFTSNGPVMQKKTLGWEAF
TETLYPADGGLEGRNDMALKLVGGSHLIANIKTTYRSKKPAKNLK
MPGVYYVDYRLERIKEANNETYVEQHEVAVARYCDLPSKLGHKLN*

### Example 10

Fusion of an Additional Binding Domain to Localize Inducer to iADAR and Increase Sensitivity

In some embodiments, an antigen activated iADAR system can be relatively insensitive to low concentrations of inducer, as the intramolecular interaction is more likely to form, especially if the inducer binding is of similar affinity, due to high local concentration (see e.g., FIG. 19A).

In order to increase the sensitivity of the antigen-activated iADAR system, described herein is a system where there are 2 or more antigen binding domains (see e.g., FIG. 19B): (1) One antigen-binding domain is bound to the intramolecular epitope mimic, leading to inactivation of ADAR (green figures). (2) The other antigen-binding domain (which binds a distinct epitope) is unbound and therefore free to bind its epitope (blue figures).

In this system, the free antigen binding domain is able to bind to the inducer and localize the activating epitope closer to the iADAR.

This system is thermodynamically more likely to open and activate the ADAR at lower concentrations, and should be more sensitive to intracellular antigen.

This second antigen binding domain can be located at any position in the fusion protein relative to the other domains.

### Example 11

Non-Limiting Examples of dsRNA Stop Loops with RNA Motifs

### UAG-UAG Stop Loop w MS2 Loop

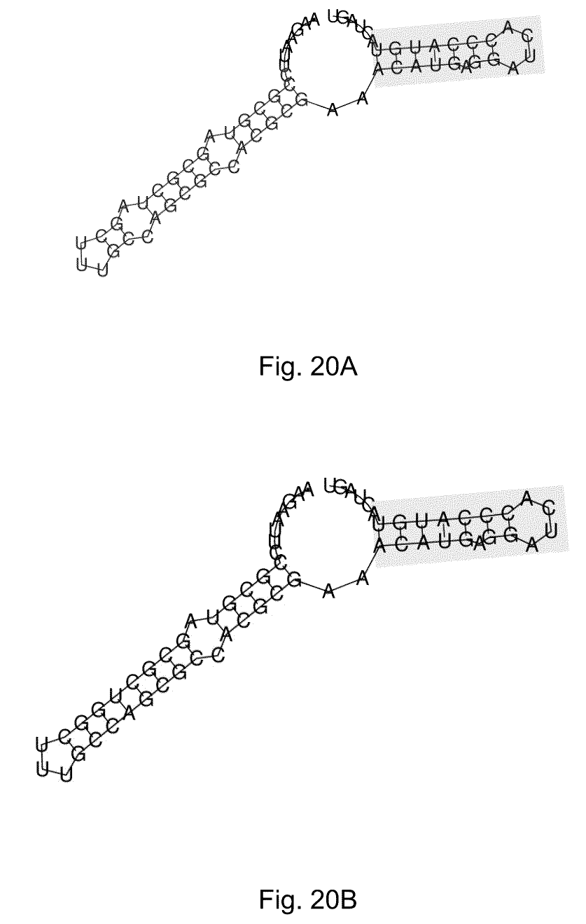

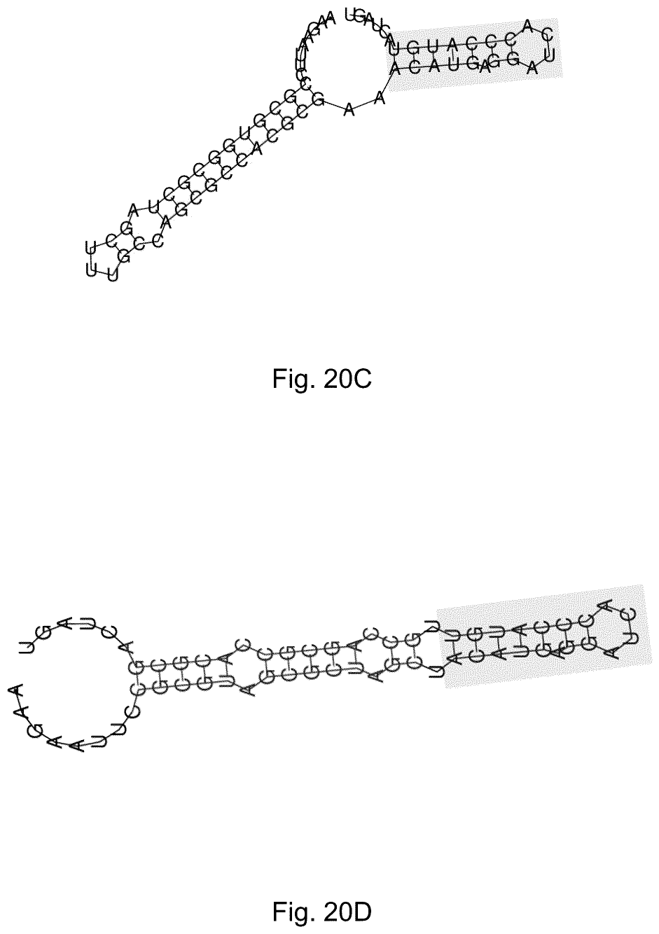

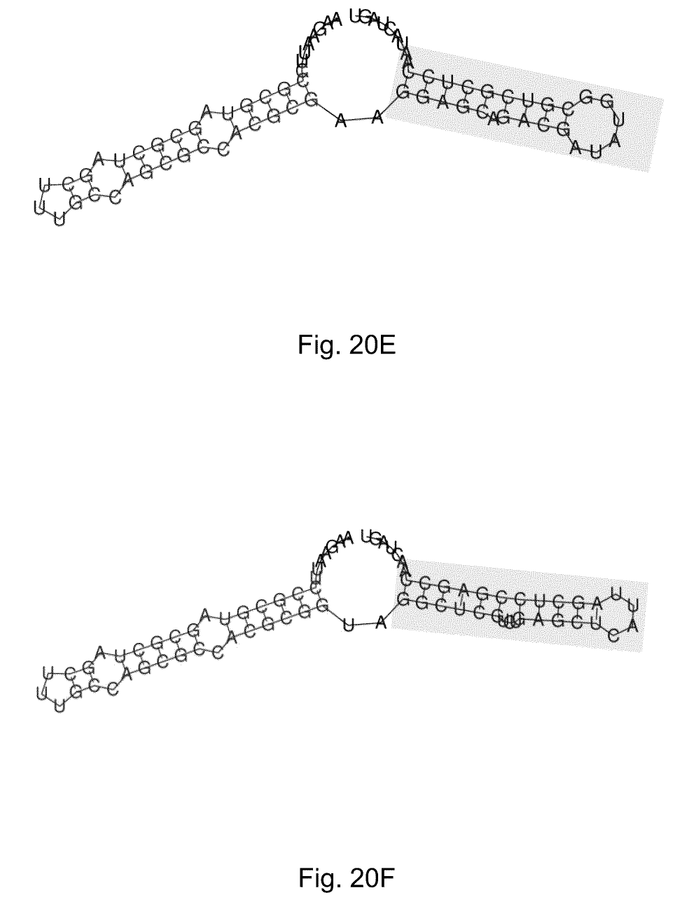

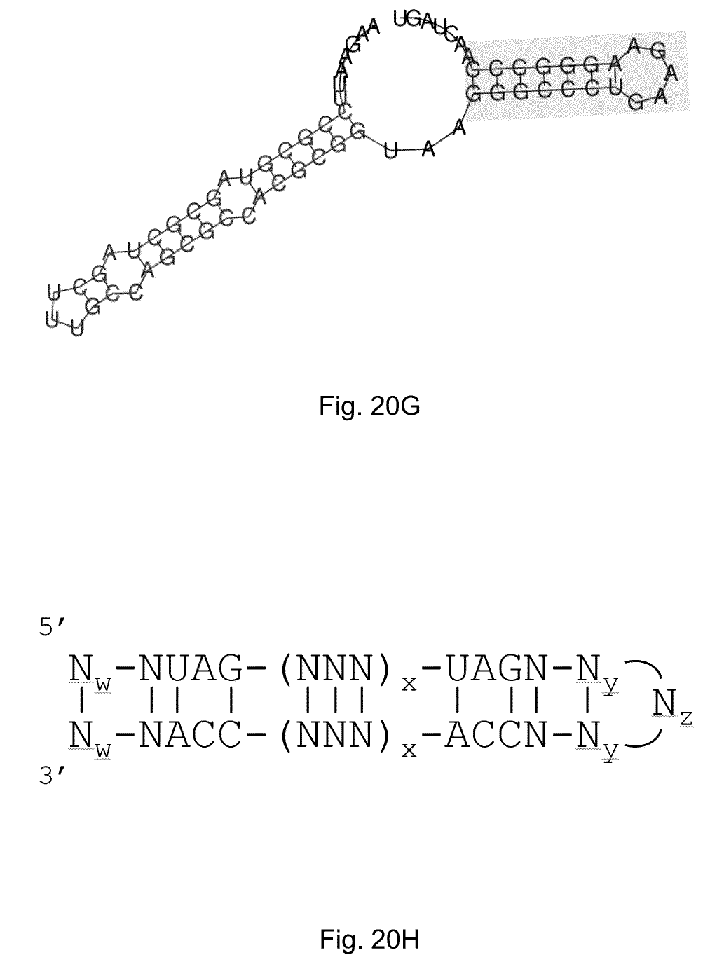

UAG-UAG Stop Loop (bolded) w/MS2 Loop (italicized); this is the loop used most frequently and for most/all of the data with MS2 loops; see e.g., FIG. 20A:

SEQ ID NO: 170, DNA-
AATTCCGCGTAGCGCTAGCTTTGCCAGCGCCACGCGaaACA
TGAGGATcACCCATGTACTAGT
SEQ ID NO: 171, RNA-
AAUUCCGCGUAGCGCUAGCUUUGCCAGCGCCACGCGaaACA
UGAGGAUcACCCAUGUACUAGU
SEQ ID NO: 172, Protein-
NSA*R*LCQRHAKHEDHPCTS
SEQ ID NO: 173, Full DNA Sequence of
mCherry_FLAG_P2A_T2A_loop
(UAG-UAG)_MS2(C)_P2A_T2A_HA_
mNeonGreen (BOLD IS ORF); bold double
underlined is UAG-UAG Stop Loop w/ MS2
Loop, SEQ ID NO: 170 above:
TAATACGACTCACTATAGGGAGACC
CAAGCTGGCTAGGTAAGCTTGGTACCGAGCTCGGATC
Caccggtcgccaccatggtgagcaagggcgaggaggata
acatggccatcatcaaggagttcatgcgcttcaaggtgca
catggagggctccgtgaacggccacgagttcgagatcgag
ggcgagggcgagggccgcccctacgagggcacccagaccg
ccaagctgaaggtgaccaagggtggccccctgcccttcgc
ctgggacatcctgtcccctcagttcatgtacggctccaag
gcctacgtgaagcaccccgccgacatccccgactacttga
agctgtccttccccgagggcttcaagtgggagcgcgtgat
gaacttcgaggacggcggcgtggtgaccgtgacccaggac
tcctccctgcaggacggcgagttcatctacaaggtgaagc
tgcgcggcaccaacttcccctccgacggccccgtaatgca
gaagaagaccatgggctgggaggcctcctccgagcggatg
taccccgaggacggcgccctgaagggcgagatcaagcaga
ggctgaagctgaaggacggcggccactacgacgctgaggt
caagaccacctacaaggccaagaagcccgtgcagctgccc
ggcgcctacaacgtcaacatcaagttggacatcacctccc
acaacgaggactacaccatcgtggaacagtacgaacgcgc
cgagggccgccactccaccggcggcatggacgagctgTAC
aaggattacaaggatgacgatgacaaaGGTAGCGGGGCAA
CTAATTTTAGCTTACTCAAACAGGCTGGGGACGTCGAGGA
GAATCCAGGCCCTGCATCCGCTGGCTCTGGAGAAGGACGA
GGCTCCTTGCTCACCTGTGGAGATGTCGAAGAGAACCCAG
GTCCTGCAACCGGGAATTCCGCGTAGCGCTAGCTTTGCCA
GCGCCACGCGaaACATGAGGATcACCCATGTACTAGTGCC
ACAAACTTCTCTCTGCTAAAGCAAGCAGGTGATGTTGAAG
AAAACCCAGGGCCTGGAGGGTCCGAGGGCAGGGGAAGTCT
CCTAACATGCGGGGACGTGGAGGAAAATCCCGGCCCATCC
GGATATCCCTACGATGTGCCCGATTACGCTCATatggtga
gcaagggcgaggaggataacatggcctctctcccagcgac
acatgagttacacatctttggctccatcaacggtgtggac
tttgacatggtgggtcagggcaccggcaatccaaatgatg
gttatgaggagttaaacctgaagtccaccaagggtgacct
ccagttctccccctggattctggtccctcatatcgggtat
ggcttccatcagtacctgccctaccctgacgggatgtcgc
ctttccaggccgccatggtagatggcAGCggataccaagt
ccatcgcacaatgcagtttgaagatggtgcctcccttact
gttaactaccgctacacctacgagggaagccacatcaaag
gagaggcccaggtgaaggggactggtttccctgctgacgg
tcctgtgatgaccaactcgctgaccgctgcggactggtgc
aggtcgaagaagacttaccccaacgacaaaaccatcatca
gtacctttaagtggagttacaccactggaaatggcaagAG
Ataccggagcactgcgcggaccacctacacctttgccaag
ccaatggcggctaactatctgaagaaccagccgatgtacg
tgttccgtaagacggagctcaagcactccaagaccgagct
caacttcaaggagtggcaaaaggcctttaccgatgtgatg
GGAatggacGAGCTGTATaagGCTAGCTAAGCGGCCGCTC
GAGTCTAGAGGGCCCGCGGTTCGAAGGTAAGCCTATCCCT
AACCCTCTCCTCGGTCTCGATTCTACGCGTACCGGTCATC
ATCACCATCACCATTGAGTTTAAACCCGCTGATCAGCCTC
GACTGTGCCTTCTA

### UAG-UGG Stop Loop w/ MS2 Loop

UAG-UGG Stop Loop (bolded) w/ MS2 Loop
(italicized); see e.g., FIG. 20B:
SEQ ID NO: 174, DNA-
AATTCCGCGTAGCGCTGGCTTTGCCAGCGCCACGCG
aaACATGAGGATcACCCATGTACTAGT
SEQ ID NO: 175, RNA-
AAUUCCGCGUAGCGCUGGCUUUGCCAGCGCCACGCG
aaACAUGAGGAUcACCCAUGUACUAGU
SEQ ID NO: 176, Protein-
NSA*RWLCQRHAKHEDHPCTS
SEQ ID NO: 177, Full DNA Sequence of mCherry_
FLAG_P2A_T2A_loop(UAG-UGG)_MS2(C)_P2A_T2A HA_
mNeonGreen; bold is ORF; bold double
underlined is UAG-UGG Stop
Loop w/ MS2 Loop, SEQ ID NO: 174 from above:
TAATACGACTCACTATAGGGAGACCCAAGCTGGCTAGGTA
AGCTTGGTACCGAGCTCGGATCCaccggtcgccaccatgg
tgagcaagggcgaggaggataacatggccatcatcaagga
gttcatgcgcttcaaggtgcacatggagggctccgtgaac
ggccacgagttcgagatcgagggcgagggcgagggccgcc
cctacgagggcacccagaccgccaagctgaaggtgaccaa
gggtggccccctgcccttcgcctgggacatcctgtcccct
cagttcatgtacggctccaaggcctacgtgaagcaccccg
ccgacatccccgactacttgaagctgtccttccccgaggg
cttcaagtgggagcgcgtgatgaacttcgaggacggcggc
gtggtgaccgtgacccaggactcctccctgcaggacggcg
agttcatctacaaggtgaagctgcgcggcaccaacttccc
ctccgacggccccgtaatgcagaagaagaccatgggctgg
gaggcctcctccgagcggatgtaccccgaggacggcgccc
tgaagggcgagatcaagcagaggctgaagctgaaggacgg
cggccactacgacgctgaggtcaagaccacctacaaggcc
aagaagcccgtgcagctgcccggcgcctacaacgtcaaca
tcaagttggacatcacctcccacaacgaggactacaccat
cgtggaacagtacgaacgcgccgagggccgccactccacc
ggcggcatggacgagctgTACaaggattacaaggatgacg
atgacaaaGGTAGCGGGGCAACTAATTTTAGCTTACTCAA
ACAGGCTGGGGACGTCGAGGAGAATCCAGGCCCTGCATCC
GCTGGCTCTGGAGAAGGACGAGGCTCCTTGCTCACCTGTG
GAGATGTCGAAGAGAACCCAGGTCCTGCAACCGGGAATTC
CGCGTAGCGCTGGCTTTGCCAGCGCCACGCGaaACATGAG
GATcACCCATGTACTAGTGCCACAAACTTCTCTCTGCTAA
AGCAAGCAGGTGATGTTGAAGAAAACCCAGGGCCTGGAGG
GTCCGAGGGCAGGGGAAGTCTCCTAACATGCGGGGACGTG
GAGGAAAATCCCGGCCCATCCGGATATCCCTACGATGTGC
CCGATTACGCTCATatggtgagcaagggcgaggaggataa
catggcctctctcccagcgacacatgagttacacatcttt
ggctccatcaacggtgtggactttgacatggtgggtcagg
gcaccggcaatccaaatgatggttatgaggagttaaacct
gaagtccaccaagggtgacctccagttctccccctggatt
ctggtccctcatatcgggtatggcttccatcagtacctgc
cctaccctgacgggatgtcgcctttccaggccgccatggt
agatggcAGCggataccaagtccatcgcacaatgcagttt
gaagatggtgcctcccttactgttaactaccgctacacct
acgagggaagccacatcaaaggagaggcccaggtgaaggg
gactggtttccctgctgacggtcctgtgatgaccaactcg
ctgaccgctgcggactggtgcaggtcgaagaagacttacc
ccaacgacaaaaccatcatcagtacctttaagtggagtta
caccactggaaatggcaagAGAtaccggagcactgcgcgg
accacctacacctttgccaagccaatggcggctaactatc
tgaagaaccagccgatgtacgtgttccgtaagacggagct
caagcactccaagaccgagctcaacttcaaggagtggcaa
aaggcctttaccgatgtgatgGGAatggacGAGCTGTATa
agGCTAGCTAAGCGGCCGCTCGAGTCTAGAGGGCCCGCGG
TTCGAAGGTAAGCCTATCCCTAACCCTCTCCTCGGTCTCG
ATTCTACGCGTACCGGTCATCATCACCATCACCATTGAGT
TTAAACCCGCTGATCAGCCTCGACTGTGCCTTCT

UGG-UAG Stop Loop (bolded) w/ MS2 Loop

UGG-UAG Stop Loop (bolded) w/ MS2 Loop
(italicized); see e.g., FIG. 20C:
SEQ ID NO: 178, DNA-
AATTCCGCGTGGCGCTAGCTTTGCCAGCGCCACGCG
aaACATGAGGATcACCCATGTACTAGT
SEQ ID NO: 179, RNA-
AAUUCCGCGUGGCGCUAGCUUUGCCAGCGCCACGCG
aaACAUGAGGAUcACCCAUGUACUAGU
SEQ ID NO: 180 and SEQ ID NO: 394, Protein-
NSAWR*LCQRHAKHEDHPCTS
SEQ ID NO: 181, Full DNA Sequence of
mCherry_FLAG_P2A_T2A_loop(UGG-UAG)_MS2(C)_
P2A_T2A HA_mNeonGreen; bold is ORF;
bold double underlined is UGG-UAG Stop
Loop w/ MS2 Loop, SEQ ID NO: 178 from above:
TAATACGACTCACTATAGGGAGACCCAAGCTGGCTAGGTA
AGCTTGGTACCGAGCTCGGATCCaccggtcgccaccatg
gtgagcaagggcgaggaggataacatggccatcatcaagg
agttcatgcgcttcaaggtgcacatggagggctccgtgaa
cggccacgagttcgagatcgagggcgagggcgagggccgc
ccctacgagggcacccagaccgccaagctgaaggtgacca
agggtggccccctgcccttcgcctgggacatcctgtcccc
tcagttcatgtacggctccaaggcctacgtgaagcacccc
gccgacatccccgactacttgaagctgtccttccccgagg
gcttcaagtgggagcgcgtgatgaacttcgaggacggcgg
cgtggtgaccgtgacccaggactcctccctgcaggacggc
gagttcatctacaaggtgaagctgcgcggcaccaacttcc
cctccgacggccccgtaatgcagaagaagaccatgggctg
ggaggcctcctccgagcggatgtaccccgaggacggcgcc
ctgaagggcgagatcaagcagaggctgaagctgaaggacg
gcggccactacgacgctgaggtcaagaccacctacaaggc
caagaagcccgtgcagctgcccggcgcctacaacgtcaac
atcaagttggacatcacctcccacaacgaggactacacca
tcgtggaacagtacgaacgcgccgagggccgccactccac
cggcggcatggacgagctgTACaaggattacaaggatgac
gatgacaaaGGTAGCGGGGCAACTAATTTTAGCTTACTCA
AACAGGCTGGGGACGTCGAGGAGAATCCAGGCCCTGCATC
CGCTGGCTCTGGAGAAGGACGAGGCTCCTTGCTCACCTGT
GGAGATGTCGAAGAGAACCCAGGTCCTGCAACCGGGAATT
CCGCGTGGCGCTAGCTTTGCCAGCGCCACGCGaaACATGA
GGATcACCCATGTACTAGTGCCACAAACTTCTCTCTGCTA
AAGCAAGCAGGTGATGTTGAAGAAAACCCAGGGCCTGGAG
GGTCCGAGGGCAGGGGAAGTCTCCTAACATGCGGGGACGT
GGAGGAAAATCCCGGCCCATCCGGATATCCCTACGATGTG
CCCGATTACGCTCATatggtgagcaagggcgaggaggata
acatggcctctctcccagcgacacatgagttacacatctt
tggctccatcaacggtgtggactttgacatggtgggtcag
ggcaccggcaatccaaatgatggttatgaggagttaaacc
tgaagtccaccaagggtgacctccagttctccccctggat
tctggtccctcatatcgggtatggcttccatcagtacctg
ccctaccctgacgggatgtcgcctttccaggccgccatgg
tagatggcAGCggataccaagtccatcgcacaatgcagtt
tgaagatggtgcctcccttactgttaactaccgctacacc
tacgagggaagccacatcaaaggagaggcccaggtgaagg
ggactggtttccctgctgacggtcctgtgatgaccaactc
gctgaccgctgcggactggtgcaggtcgaagaagacttac
cccaacgacaaaaccatcatcagtacctttaagtggagtt
acaccactggaaatggcaagAGAtaccggagcactgcgcg
gaccacctacacctttgccaagccaatggcggctaactat
ctgaagaaccagccgatgtacgtgttccgtaagacggagc
tcaagcactccaagaccgagctcaacttcaaggagtggca
aaaggcctttaccgatgtgatgGGAatggacGAGCTGTAT
aagGCTAGCTAAGCGGCCGCTCGAGTCTAGAGGGCCCGCG
GTTCGAAGGTAAGCCTATCCCTAACCCTCTCCTCGGTCTC
GATTCTACGCGTACCGGTCATCATCACCATCACCATTGAG
TTTAAACCCGCTGATCAGCCTCGACTGTGCCTTCT

### UAG-UAG Stop Loop w/ Internal MS2 Loop

UAG-UAG Stop Loop (bolded) w/Internal MS2
Loop (italicized); see e.g., FIG. 20D:
SEQ ID NO: 182, DNA-
AATTCCGCGTAGCGCTAGCTACATGAGGATcA
CCCATGTTGCCAGCGCCACGCGACTAGT
SEQ ID NO: 183, RNA-
AAUUCCGCGUAGCGCUAGCUACAUGAGGAUCA
CCCAUGUUGCCAGCGCCACGCGACUAGU
SEQ ID NO: 184, Protein-
NSA*R*LHEDHPCCQRHATS
SEQ ID NO: 185, Full DNA Sequence of
mCherry FLAG_P2A_T2A_loop((UAG-UAG)
MS2(C))_P2A_T2A_HA_mNeonGreen;
bold is ORF; bold double underlined
is UAG-UAG Stop Loop w/Internal MS2
Loop, SEQ ID NO: 182 from above:
TAATACGACTCACTATAGGGAGACCCAAGCTGGCTAGGTA
AGCTTGGTACCGAGCTCGGATCCaccggtcgccaccatgg
tgagcaagggcgaggaggataacatggccatcatcaagga
gttcatgcgcttcaaggtgcacatggagggctccgtgaac
ggccacgagttcgagatcgagggcgagggcgagggccgcc
cctacgagggcacccagaccgccaagctgaaggtgaccaa
gggtggccccctgcccttcgcctgggacatcctgtcccct
cagttcatgtacggctccaaggcctacgtgaagcaccccg
ccgacatccccgactacttgaagctgtccttccccgaggg
cttcaagtgggagcgcgtgatgaacttcgaggacggcggc
gtggtgaccgtgacccaggactcctccctgcaggacggcg
agttcatctacaaggtgaagctgcgcggcaccaacttccc
ctccgacggccccgtaatgcagaagaagaccatgggctgg
gaggcctcctccgagcggatgtaccccgaggacggcgccc
tgaagggcgagatcaagcagaggctgaagctgaaggacgg
cggccactacgacgctgaggtcaagaccacctacaaggcc
aagaagcccgtgcagctgcccggcgcctacaacgtcaaca
tcaagttggacatcacctcccacaacgaggactacaccat
cgtggaacagtacgaacgcgccgagggccgccactccacc
ggcggcatggacgagctgTACaaggattacaaggatgacg
atgacaaaGGTAGCGGGGCAACTAATTTTAGCTTACTCAA
ACAGGCTGGGGACGTCGAGGAGAATCCAGGCCCTGCATCC
GCTGGCTCTGGAGAAGGACGAGGCTCCTTGCTCACCTGTG
GAGATGTCGAAGAGAACCCAGGTCCTGCAACCGGGAATTC
CGCGTAGCGCTAGCTACATGAGGATcACCCATGTTGCCAG
CGCCACGCGACTAGTGCCACAAACTTCTCTCTGCTAAAGC
AAGCAGGTGATGTTGAAGAAAACCCAGGGCCTGGAGGGTC
CGAGGGCAGGGGAAGTCTCCTAACATGCGGGGACGTGGAG
GAAAATCCCGGCCCATCCGGATATCCCTACGATGTGCCCG
ATTACGCTCATatggtgagcaagggcgaggaggataacat
ggcctctctcccagcgacacatgagttacacatctttggc
tccatcaacggtgtggactttgacatggtgggtcagggca
ccggcaatccaaatgatggttatgaggagttaaacctgaa
gtccaccaagggtgacctccagttctccccctggattctg
gtccctcatatcgggtatggcttccatcagtacctgccct
accctgacgggatgtcgcctttccaggccgccatggtaga
tggcAGCggataccaagtccatcgcacaatgcagtttgaa
gatggtgcctcccttactgttaactaccgctacacctacg
agggaagccacatcaaaggagaggcccaggtgaaggggac
tggtttccctgctgacggtcctgtgatgaccaactcgctg
accgctgcggactggtgcaggtcgaagaagacttacccca
acgacaaaaccatcatcagtacctttaagtggagttacac
cactggaaatggcaagAGAtaccggagcactgcgcggacc
acctacacctttgccaagccaatggcggctaactatctga
agaaccagccgatgtacgtgttccgtaagacggagctcaa
gcactccaagaccgagctcaacttcaaggagtggcaaaag
gcctttaccgatgtgatgGGAatggacGAGCTGTATaagG
CTAGCTAAGCGGCCGCTCGAGTCTAGAGGGCCCGCGGTTC
GAAGGTAAGCCTATCCCTAACCCTCTCCTCGGTCTCGATT
CTACGCGTACCGGTCATCATCACCATCACCATTGAGTTTA
AACCCGCTGATCAGCCTCGACTGTGCCTTCT

### UAG-UAG Stop Loop w/ PP7 Loop

UAG-UAG Stop Loop (bolded) w/ PP7 Loop
(italicized); see e.g., FIG. 20E:
SEQ ID NO: 186, DNA-
AATTCCGCGTAGCGCTAGCTTTGCCAGCGCCACGCG
aaggagcagacgatatggcgtcgctccaaTACTAGT
SEQ ID NO: 187, RNA-
AAUUCCGCGUAGCGCUAGCUUUGCCAGCGCCACGCG
aaggagcagacgauauggcgucgcuccaaUACUAGU
SEQ ID NO: 188, Protein-
NSA*R*LCQRHAKEQTIWRRSNTS
SEQ ID NO: 189, Full DNA Sequence of
mCherry_FLAG_P2A_T2A_loop(UAG-
UAG) PP7_P2A_T2A_HA_mNeonGreen; bold
is ORF; bold double underlined is
UAG-UAG Stop Loop w/ PP7 Loop,
SEQ ID NO: 186 from above:
TAATACGACTCACTATAGGGAGACCCAAGCTGGCTAGGTA
AGCTTGGTACCGAGCTCGGATCCaccggtcgccaccatg
gtgagcaagggcgaggaggataacatggccatcatcaagg
agttcatgcgcttcaaggtgcacatggagggctccgtgaa
cggccacgagttcgagatcgagggcgagggcgagggccgc
ccctacgagggcacccagaccgccaagctgaaggtgacca
agggtggccccctgcccttcgcctgggacatcctgtcccc
tcagttcatgtacggctccaaggcctacgtgaagcacccc
gccgacatccccgactacttgaagctgtccttccccgagg
gcttcaagtgggagcgcgtgatgaacttcgaggacggcgg
cgtggtgaccgtgacccaggactcctccctgcaggacggc
gagttcatctacaaggtgaagctgcgcggcaccaacttcc
cctccgacggccccgtaatgcagaagaagaccatgggctg
ggaggcctcctccgagcggatgtaccccgaggacggcgcc
ctgaagggcgagatcaagcagaggctgaagctgaaggacg
gcggccactacgacgctgaggtcaagaccacctacaaggc
caagaagcccgtgcagctgcccggcgcctacaacgtcaac
atcaagttggacatcacctcccacaacgaggactacacca
tcgtggaacagtacgaacgcgccgagggccgccactccac
cggcggcatggacgagctgTACaaggattacaaggatgac
gatgacaaaGGTAGCGGGGCAACTAATTTTAGCTTACTCA
AACAGGCTGGGGACGTCGAGGAGAATCCAGGCCCTGCATC
CGCTGGCTCTGGAGAAGGACGAGGCTCCTTGCTCACCTGT
GGAGATGTCGAAGAGAACCCAGGTCCTGCAACCGGGAATT
CCGCGTAGCGCTAGCTTTGCCAGCGCCACGCGaaggagca
gacgatatggcctcgctccaaTACTAGTGCCACAAACTTC
TCTCTGCTAAAGCAAGCAGGTGATGTTGAAGAAAACCCAG
GGCCTGGAGGGTCCGAGGGCAGGGGAAGTCTCCTAACATG
CGGGGACGTGGAGGAAAATCCCGGCCCATCCGGATATCCC
TACGATGTGCCCGATTACGCTCATatggtgagcaagggcg
aggaggataacatggcctctctcccagcgacacatgagtt
acacatctttggctccatcaacggtgtggactttgacatg
gtgggtcagggcaccggcaatccaaatgatggttatgagg
agttaaacctgaagtccaccaagggtgacctccagttctc
cccctggattctggtccctcatatcgggtatggcttccat
cagtacctgccctaccctgacgggatgtcgcctttccagg
ccgccatggtagatggcAGCggataccaagtccatcgcac
aatgcagtttgaagatggtgcctcccttactgttaactac
cgctacacctacgagggaagccacatcaaaggagaggccc
aggtgaaggggactggtttccctgctgacggtcctgtgat
gaccaactcgctgaccgctgcggactggtgcaggtcgaag
aagacttaccccaacgacaaaaccatcatcagtaccttta
agtggagttacaccactggaaatggcaagAGAtaccggag
cactgcgcggaccacctacacctttgccaagccaatggcg
gctaactatctgaagaaccagccgatgtacgtgttccgta
agacggagctcaagcactccaagaccgagctcaacttcaa
ggagtggcaaaaggcctttaccgatgtgatgGGAatggac
GAGCTGTATaagGCTAGCTAAGCGGCCGCTCGAGTCTAGA
GGGCCCGCGGTTCGAAGGTAAGCCTATCCCTAACCCTCTC
CTCGGTCTCGATTCTACGCGTACCGGTCATCATCACCATC
ACCATTGAGTTTAAACCCGCTGATCAGCCTCGACTGTGCC
TTCT

### UAG-UAG Stop Loop w/ HIV Tar Loop

UAG-UAG Stop Loop (bolded) w/ HIV Tar
Loop (italicized); see e.g., FIG. 20F:
SEQ ID NO: 190, DNA-
AATTCCGCGTAGCGCTAGCTTTGCCAGCGCCACGCGGt
aggctcgtctgagctcattagctccgagccaACTAGT
SEQ ID NO: 191, RNA-
AAUUCCGCGUAGCGCUAGCUUUGCCAGCGCCACGCGGua
ggcucgucugagcucauuagcuccgagccaACUAGU
SEQ ID NO: 192, Protein-
NSA*R*LCQRHAVGSSELISSEPTS
SEQ ID NO: 193, Full DNA Sequence of
mCherry FLAG_P2A_T2A loop(UAG-UAG)
HIV Tar P2A T2A HA mNeonGreen; bold is ORF;
bold double underlined is UAG-UAG Stop
Loop w/ HIV Tar Loop, SEQ ID NO: 190 from above:
TAATACGACTCACTATAGGGAGACCCAAGCTGGCTAGGTAAGCTT
GGTACCGAGCTCGGATCCaccggtcgccaccatggtgagcaaggg
cgaggaggataacatggccatcatcaaggagttcatgcgcttcaa
ggtgcacatggagggctccgtgaacggccacgagttcgagatcga
gggcgagggcgagggccgcccctacgagggcacccagaccgccaa
gctgaaggtgaccaagggtggccccctgcccttcgcctgggacat
cctgtcccctcagttcatgtacggctccaaggcctacgtgaagca
ccccgccgacatccccgactacttgaagctgtccttccccgaggg
cttcaagtgggagcgcgtgatgaacttcgaggacggcggcgtggt
gaccgtgacccaggactcctccctgcaggacggcgagttcatcta
caaggtgaagctgcgcggcaccaacttcccctccgacggccccgt
aatgcagaagaagaccatgggctgggaggcctcctccgagcggat
gtaccccgaggacggcgccctgaagggcgagatcaagcagaggct
gaagctgaaggacggcggccactacgacgctgaggtcaagaccac
ctacaaggccaagaagcccgtgcagctgcccggcgcctacaacgt
caacatcaagttggacatcacctcccacaacgaggactacaccat
cgtggaacagtacgaacgcgccgagggccgccactccaccggcgg
catggacgagctgTACaaggattacaaggatgacgatgacaaaGG
TAGCGGGGCAACTAATTTTAGCTTACTCAAACAGGCTGGGGACGT
CGAGGAGAATCCAGGCCCTGCATCCGCTGGCTCTGGAGAAGGACG
AGGCTCCTTGCTCACCTGTGGAGATGTCGAAGAGAACCCAGGTCC
TGCAACCGGGAATTCCGCGTAGCGCTAGCTTTGCCAGCGCCACGC
GGtaggctcgtctgagctcattagctccgagccaACTAGTGCCAC
AAACTTCTCTCTGCTAAAGCAAGCAGGTGATGTTGAAGAAAACCC
AGGGCCTGGAGGGTCCGAGGGCAGGGGAAGTCTCCTAACATGCGG
GGACGTGGAGGAAAATCCCGGCCCATCCGGATATCCCTACGATGT
GCCCGATTACGCTCATatggtgagcaagggcgaggaggataacat
ggcctctctcccagcgacacatgagttacacatctttggctccat
caacggtgtggactttgacatggtgggtcagggcaccggcaatcc
aaatgatggttatgaggagttaaacctgaagtccaccaagggtga
cctccagttctccccctggattctggtccctcatatcgggtatgg
cttccatcagtacctgccctaccctgacgggatgtcgcctttcca
ggccgccatggtagatggcAGCggataccaagtccatcgcacaat
gcagtttgaagatggtgcctcccttactgttaactaccgctacac
ctacgagggaagccacatcaaaggagaggcccaggtgaaggggac
tggtttccctgctgacggtcctgtgatgaccaactcgctgaccgc
tgcggactggtgcaggtcgaagaagacttaccccaacgacaaaac
catcatcagtacctttaagtggagttacaccactggaaatggcaa
gAGAtaccggagcactgcgcggaccacctacacctttgccaagcc
aatggcggctaactatctgaagaaccagccgatgtacgtgttccg
taagacggagctcaagcactccaagaccgagctcaacttcaagga
gtggcaaaaggcctttaccgatgtgatgGGAatggacGAGCTGTA
TaagGCTAGCTAAGCGGCCGCTCGAGTCTAGAGGGCCCGCGGTTC
GAAGGTAAGCCTATCCCTAACCCTCTCCTCGGTCTCGATTCTACG
CGTACCGGTCATCATCACCATCACCATTGAGTTTAAACCCGCTGA
TCAGCCTCGACTGTGCCTTCT

### UAG-UAG Stop Loop w/ BoxB Loop

UAG-UAG Stop Loop (bolded) w/ BoxB Loop
(italicized); see e.g., FIG. 20G:
SEQ ID NO: 194, DNA-
AATTCCGCGTAGCGCTAGCTTTGCCAGCGCCACGCGG
taagggccctgaagaagggcccaACTAGT
SEQ ID NO: 195, RNA-
AAUUCCGCGUAGCGCUAGCUUUGCCAGCGCCACGCGG
uaagggcccugaagaagggcccaACUAGU
SEQ ID NO: 196, Protein-NSA*R*LCQRHAVRALKKGPTS
SEQ ID NO: 197, Full DNA Sequence
of mCherry_FLAG_P2A_T2A_loop(UAG-
UAG) BoxB_P2A_T2A HA mNeonGreen; bold is ORF;
bold double underlined is UAG-UAG Stop
Loop w/ BoxB Loop, SEQ ID NO: 194 from above:
TAATACGACTCACTATAGGGAGACCCAAGCTGGCTAGGTAAGCTT
GGTACCGAGCTCGGATCCaccggtcgccaccatggtgagcaaggg
cgaggaggataacatggccatcatcaaggagttcatgcgcttcaa
ggtgcacatggagggctccgtgaacggccacgagttcgagatcga
gggcgagggcgagggccgcccctacgagggcacccagaccgccaa
gctgaaggtgaccaagggtggccccctgcccttcgcctgggacat
cctgtcccctcagttcatgtacggctccaaggcctacgtgaagca
ccccgccgacatccccgactacttgaagctgtccttccccgaggg
cttcaagtgggagcgcgtgatgaacttcgaggacggcggcgtggt
gaccgtgacccaggactcctccctgcaggacggcgagttcatcta
caaggtgaagctgcgcggcaccaacttcccctccgacggccccgt
aatgcagaagaagaccatgggctgggaggcctcctccgagcggat
gtaccccgaggacggcgccctgaagggcgagatcaagcagaggct
gaagctgaaggacggcggccactacgacgctgaggtcaagaccac
ctacaaggccaagaagcccgtgcagctgcccggcgcctacaacgt
caacatcaagttggacatcacctcccacaacgaggactacaccat
cgtggaacagtacgaacgcgccgagggccgccactccaccggcgg
catggacgagctgTACaaggattacaaggatgacgatgacaaaGG
TAGCGGGGCAACTAATTTTAGCTTACTCAAACAGGCTGGGGACGT
CGAGGAGAATCCAGGCCCTGCATCCGCTGGCTCTGGAGAAGGACG
AGGCTCCTTGCTCACCTGTGGAGATGTCGAAGAGAACCCAGGTCC
TGCAACCGGGAATTCCGCGTAGCGCTAGCTTTGCCAGCGCCACGC
GGtaagggccctgaagaagggcccaACTAGTGCCACAAACTTCTC
TCTGCTAAAGCAAGCAGGTGATGTTGAAGAAAACCCAGGGCCTGG
AGGGTCCGAGGGCAGGGGAAGTCTCCTAACATGCGGGGACGTGGA
GGAAAATCCCGGCCCATCCGGATATCCCTACGATGTGCCCGATTA
CGCTCATatggtgagcaagggcgaggaggataacatggcctctct
cccagcgacacatgagttacacatctttggctccatcaacggtgt
ggactttgacatggtgggtcagggcaccggcaatccaaatgatgg
ttatgaggagttaaacctgaagtccaccaagggtgacctccagtt
ctccccctggattctggtccctcatatcgggtatggcttccatca
gtacctgccctaccctgacgggatgtcgcctttccaggccgccat
ggtagatggcAGCggataccaagtccatcgcacaatgcagtttga
agatggtgcctcccttactgttaactaccgctacacctacgaggg
aagccacatcaaaggagaggcccaggtgaaggggactggtttccc
tgctgacggtcctgtgatgaccaactcgctgaccgctgcggactg
gtgcaggtcgaagaagacttaccccaacgacaaaaccatcatcag
tacctttaagtggagttacaccactggaaatggcaagAGAtaccg
gagcactgcgcggaccacctacacctttgccaagccaatggcggc
taactatctgaagaaccagccgatgtacgtgttccgtaagacgga
gctcaagcactccaagaccgagctcaacttcaaggagtggcaaaa
ggcctttaccgatgtgatgGGAatggacGAGCTGTATaagGCTAG
CTAAGCGGCCGCTCGAGTCTAGAGGGCCCGCGGTTCGAAGGTAAG
CCTATCCCTAACCCTCTCCTCGGTCTCGATTCTACGCGTACCGGT
CATCATCACCATCACCATTGAGTTTAAACCCGCTGATCAGCCTCG
ACTGTGCCTTCT

### Example 12: Mutations to the IP6 Binding Pocket Reduce Background Activity

With the IP6-competition hypothesis previously outlined, the system should be in equilibrium between the cis-interaction (misfolded) state of ADAR and the IP6-bound (folded and active) state of ADAR (see e.g., FIG. 22a). In this model, the background in the system would be a result of the IP6 bound state. Background can be reduced by shifting the equilibrium towards the cis-interaction by either increasing the affinity of the heterodimers or by decreasing the affinity for the enzyme for IP6.

Towards this end, mutant variants of the ADAR2-DD were created and tested to see if the background could be reduced from a leaky system. The screen focused on conserved residues involved in IP6 binding or C-terminal tail folding (see e.g., FIG. 22b), as it was hypothesized that these would be the most likely to reduce the background of the system.

As a scaffold, a construct was used that displayed increased leakiness due to a mutation in the BAD peptide (F121V) which reduces affinity for Bcl-xL (see e.g., FIG. 22c). Mutated versions were then cloned and co-transfected with an editing reporter in the absence and presence of A-1331852, and absolute background and fold change upon drug addition were determined by flow cytometry (see e.g., FIG. 22d and table 11). In this assay, R522M, L699G, and R400K were found to yield the highest fold changes while other mutations had lower absolute background. Additional mutations and combinations were tested with different reporter constructs as well, and those are listed in table 12.

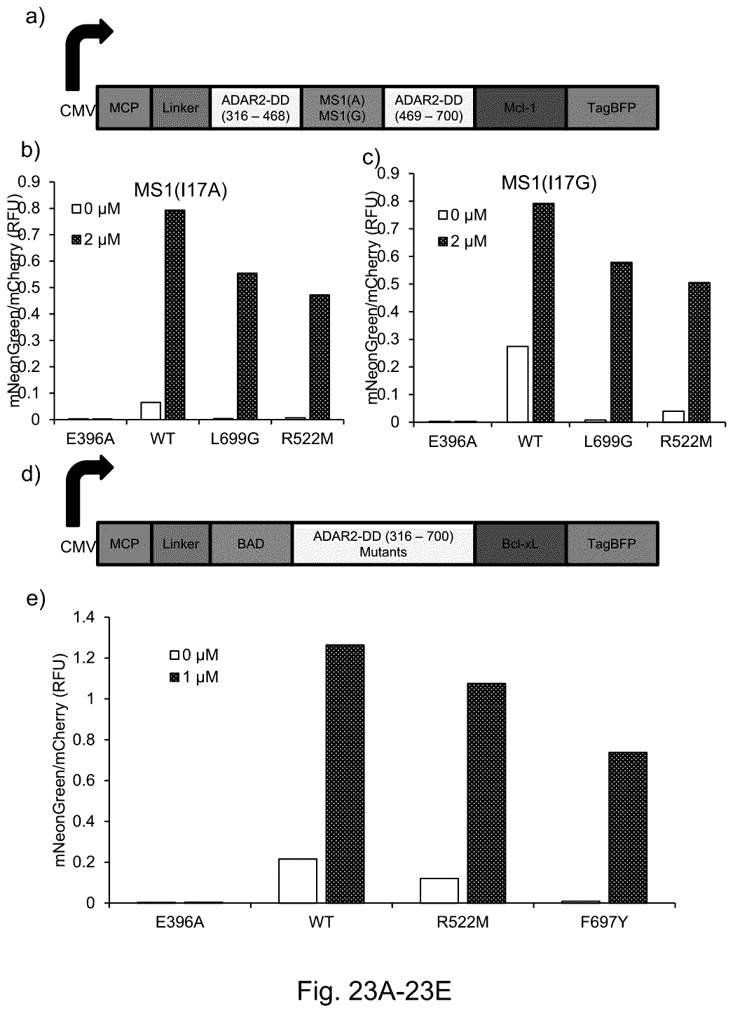

These mutations were then tested to see if they would be transferrable to other “leaky” systems. The MS1(117A)-Mcl1 interaction pair was previously found to lead to a drug (S63845) inducible ADAR, but there was substantial background in the absence of the drug (see e.g., FIG. 23a). The R522M and L699G mutations were then cloned into the ADAR-DD and the plasmids were co-transfected into HEK cells with and without S63845. Both R522M and L699G mutations substantially reduced the background of the MS1(A)-Mcl1 system while not seriously decreasing the activation (see e.g., FIG. 23b). This resulted in an increase in fold change from 12× for the WT DD to 67× and 140× for R522M and L699G respectively. These mutations were also tested in the even leaker MS1(I17G) system and substantial reduction was found in background (see e.g., FIG. 23c). The fold change increased from 2.8× for the WT DD to 13× and 73× for R522M and L699G respectively. These data demonstrate that these mutations can be useful in multiple drug controlled systems.

These or other mutations were then tested to see if they could reduce the background in the N-terminal system (see e.g., FIG. 23d). HEK cells were co-transfected with both the ADAR-DD encoding plasmid and the reporter plasmid in the absence or presence of the Bcl-xL inhibitor A-1331852. The F697Y mutation substantially decreased the background of the system and therefore led to a substantial increase in fold change (5.9× for WT and 83× for F697Y). This improved N-terminal fusion scaffold is great utility for using globular/folded heterodimeric pairs (e.g., the use of an antibody fragment and its epitope for antigen sensing).

TABLE 11
Mutation List and Corresponding Flow Cytometry Values

| | | | Fold |
|---|---|---|---|
| NAME | 0 μM | 1 μM | Change |
| E396A | 0.0049 | 0.0047 | 0.964933 |
| DD Only | 0.9853 | 1.0147 | 1.02994 |
| BAD(L) | 0.690265 | 0.672566 | 0.974359 |
| Only | | | |
| BAD(F) | 0.025959 | 0.961652 | 37.04545 |
| BAD(L) | 0.038938 | 0.902655 | 23.18182 |
| BAD(V) | 0.070796 | 1.020649 | 14.41667 |
| BAD(G) | 0.100295 | 0.967552 | 9.647059 |
| Mutations | | | |
| T375G | 0.004454 | 0.016519 | 3.708609 |
| R400K | 0.015339 | 0.560472 | 36.53846 |
| R522M | 0.014749 | 0.619469 | 42 |
| K662R | 0.005457 | 0.016519 | 3.027027 |
| K662M | 0.004879 | 0.0059 | 1.20919 |
| K672R | 0.00885 | 0.230088 | 26 |
| K672M | 0.0059 | 0.049558 | 8.4 |
| V688A | 0.005481 | 0.00826 | 1.506997 |
| V688G | 0.00826 | 0.054867 | 6.642857 |
| K690R | 0.033038 | 0.926254 | 28.03571 |
| K690M | 0.057817 | 0.932153 | 16.12245 |
| F697Y | 0.00649 | 0.106195 | 16.36364 |
| F697L | 0.00708 | 0.088496 | 12.5 |
| F697I | 0.005552 | 0.076696 | 13.81509 |
| F697V | 0.005027 | 0.018879 | 3.755869 |
| F697A | 0.005528 | 0.00944 | 1.707577 |
| F697G | 0.00649 | 0.011209 | 1.727273 |
| L699V | 0.056047 | 0.80826 | 14.42105 |
| L699A | 0.038348 | 0.725664 | 18.92308 |
| L699G | 0.018289 | 0.637168 | 34.83871 |

TABLE 12
Additional mutations tested, but no data shown.

| Original Residue | Mutated Residues |
|---|---|
| S531 | S531A |
| F697 | F697M, F697W, F697H |
| Y658 | Y658F |
| Y668 | Y668F |

TABLE 13
Amino Acid Sequence Table (see e.g., FIG. 11,
14, 22, 23, 26); NOTE: ALL other
mutants listed in FIG. 22d are of “BAD(V)”
with the corresponding mutation

| | SEQ | | |
|---|---|---|---|
| | ID | See e.g., | |
| NAME | NO | FIGS. | AMINO ACID SEQUENCE |
| MCP-linker- | 92 | 11f, 14c, | MASNFTQFVLVDNGGTGDVTVAPSNFANGIAEWISSNSR |
| BAD- | | 23e | SQAYKVTCSVRQSSAQNRKYTIKVEVPKGAWRSYLNME |
| ADAR2- | | Note | LTIPIFATNSDCELIVKAMQGLLKDGNPIPSAIAANSGI |
| DD(E488Q) | | Mutations | YGGSGSGAGSGSPAGGGAPGSGGGSTGAPPNLWAAQRYG |
| -Bcl-xL- | | in 23e are | RELRRMSDEFVDSFKKASQLHLPQVLADAVSRLVLGKFG |
| TagBFP | | bolded | DLTDNFSSPHARRKVLAGVVMTTGTDVKDAKVISVSTGTK |
| | | | CINGEYMSDRGLALNDCHAEIISRRSLLRFLYTQLELYLN |
| | | | NKDDQKRSIFQKSERGGFRLKENVQFHLYISTSPCGDARI |
| | | | FSPHEPILEEPADRHPNRKARGQLRTKIESGQGTIPVRSNA |
| | | | SIQTWDGVLQGERLLTMSCSDKIARWNVVGIQGSLLSIFV |
| | | | EPIYFSSIILGSLYHGDHLSRAMYQRISNIEDLPPLYTLN |
| | | | KPLLSGISNAEARQPGKAPNFSVNWTVGDSAIEVINATTG |
| | | | KDELGRASRLCKHALYCRWMRVHGKVPSHLLRSKITKPNV |
| | | | YHESKLAAKEYQAAKARLFTAFIKAGLGAWVEKPTEQD |
| | | | QFSLTGSAAASSNRELVVDFLSYKLSQKGYSWSQFSDVE |
| | | | ENRTEAPEGTESEMETPSAINGNPSWHLADSPAVNGATG |
| | | | HSSSLDAREVIPMAAVKQALREAGDEFELRYRRAFSDLT |
| | | | SQLHITPGTAYQSFEQVVNELFRDGVNWGRIVAFFSFGG |
| | | | ALCVESVDKEMQVLVSRIAAWMATYLNDHLEPWIQENG |
| | | | GWDTFVELYGNNGSSELIKENMHMKLYMEGTVDNHHF |
| | | | KCTSEGEGKPYEGTQTMRIKVVEGGPLPFAFDILATSFLY |
| | | | GSKTFINHTQGIPDFFKQSFPEGFTWERVTTYEDGGVLTA |
| | | | TQDTSLQDGCLIYNVKIRGVNFTSNGPVMQKKTLGWEAF |
| | | | TETLYPADGGLEGRNDMALKLVGGSHLIANIKTTYRSKK |
| | | | PAKNLKMPGVYYVDYRLERIKEANNETYVEQHEVAVAR |
| | | | YCDLPSKLGHKLN* |
| Also known | 287 | 22d, 25a | MASNFTQFVLVDNGGTGDVTVAPSNFANGIAEWISSNSR |
| as “BAD- | | “BAD(F)” | SQAYKVTCSVRQSSAQNRKYTIKVEVPKGAWRSYLNMEL |
| DD-BclxL” | | | TIPIFATNSDCELIVKAMQGLLKDGNPIPSAIAANSGIY |
| and “WT” | | | GGSGSGAGSGSPAGGGAPGSGGGSQLHLPQVLADAVSRL |
| MCP-linker- | | | VLGKFGDLTDNFSSPHARRKVLAGVVMTTGTDVKDAKVI |
| ADAR2- | | | SVSTGTKCINGEYMSDRGLALNDCHAEIISRRSLLRFLYT |
| DDN- | | | QLELYLNNKDDQKRSIFQKSERGGFRLKENVQFHLYISTS |
| Bad(F)- | | | PCGDARIFSPHEPILEEPAASGSGTGAPPNLWAAQRYGRE |
| ADAR2(E4 | | | LRRMSDEFVDRHPNRKARGQLRTKIESGQGTIPVRSNASI |
| 88Q)-DDC- | | | QTWDGVLQGERLLTMSCSDKIARWNVVGIQGSLLSIFVEP |
| Bcl-xL- | | | IYFSSIILGSLYHGDHLSRAMYQRISNIEDLPPLYTLNKP |
| TagBFP | | | LLSGISNAEARQPGKAPNFSVNWTVGDSAIEVINATTGKD |
| | | | ELGRASRLCKHALYCRWMRVHGKVPSHLLRSKITKPNVY |
| | | | HESKLAAKEYQAAKARLFTAFIKAGLGAWVEKPTEQDQ |
| | | | FSLTGSAAGGSGGSAAASSNRELVVDFLSYKLSQKGYSW |
| | | | SQFSDVEENRTEAPEGTESEMETPSAINGNPSWHLADSPA |
| | | | VNGATGHSSSLDAREVIPMAAVKQALREAGDEFELRYRR |
| | | | AFSDLTSQLHITPGTAYQSFEQVVNELFRDGVNWGRIVAF |
| | | | FSFGGALCVESVDKEMQVLVSRIAAWMATYLNDHLEPW |
| | | | IQENGGWDTFVELYGNNGSSELIKENMHMKLYMEGTVD |
| | | | NHHFKCTSEGEGKPYEGTQTMRIKVVEGGPLPFAFDILAT |
| | | | SFLYGSKTFINHTQGIPDFFKQSFPEGFTWERVTTYEDGG |
| | | | VLTATQDTSLQDGCLIYNVKIRGVNFTSNGPVMQKKTLG |
| | | | WEAFTETLYPADGGLEGRNDMALKLVGGSHLIANIKTTY |
| | | | RSKKPAKNLKMPGVYYVDYRLERIKEANNETYVEQHEV |
| | | | AVARYCDLPSKLGHKLN* |
| MCP-linker- | 288 | 22d, 25a | MASNFTQFVLVDNGGTGDVTVAPSNFANGIAEWISSNSR |
| ADAR2- | | “BAD(V)” | SQAYKVTCSVRQSSAQNRKYTIKVEVPKGAWRSYLNMEL |
| DDN- | | | TIPIFATNSDCELIVKAMQGLLKDGNPIPSAIAANSGIY |
| Bad(F)- | | | GGSGSGAGSGSPAGGGAPGSGGGSQLHLPQVLADAVSRL |
| ADAR2(E4 | | | VLGKFGDLTDNFSSPHARRKVLAGVVMTTGTDVKDAKVI |
| 88Q)-DDC- | | | SVSTGTKCINGEYMSDRGLALNDCHAEIISRRSLLRFLYT |
| | | | QLELYLNNKDDQKRSIFQKSERGGFRLKENVQFHLYISTS |
| | | | PCGDARIFSPHEPILEEPAASGSGTGAPPNLWAAQRYGRE |
| | | | LRRMSDEVVDRHPNRKARGQLRTKIESGQGTIPVRSNASI |
| | | | QTWDGVLQGERLLTMSCSDKIARWNVVGIQGSLLSIFVE |
| | | | PIYFSSIILGSLYHGDHLSRAMYQRISNIEDLPPLYTLNK |
| | | | PLLSGISNAEARQPGKAPNFSVNWTVGDSAIEVINATTGK |
| | | | DELGRASRLCKHALYCRWMRVHGKVPSHLLRSKITKPNVY |
| | | | HESKLAAKEYQAAKARLFTAFIKAGLGAWVEKPTEQDQF |
| | | | SLTGSAAGGSGGSAAASSNRELVVDFLSYKLSQKGYSWS |
| | | | QFSDVEENRTEAPEGTESEMETPSAINGNPSWHLADSPA |
| | | | VNGATGHSSSLDAREVIPMAAVKQALREAGDEFELRYRR |
| | | | AFSDLTSQLHITPGTAYQSFEQVVNELFRDGVNWGRIVA |
| | | | FFSFGGALCVESVDKEMQVLVSRIAAWMATYLNDHLEPW |
| | | | IQENGGWDTFVELYGNNGSSELIKENMHMKLYMEGTVDN |
| | | | HHFKCTSEGEGKPYEGTQTMRIKVVEGGPLPFAFDILAT |
| | | | SFLYGSKTFINHTQGIPDFFKQSFPEGFTWERVTTYEDGG |
| | | | VLTATQDTSLQDGCLIYNVKIRGVNFTSNGPVMQKKTLG |
| | | | WEAFTETLYPADGGLEGRNDMALKLVGGSHLIANIKTTY |
| | | | RSKKPAKNLKMPGVYYVDYRLERIKEANNETYVEQHEV |
| | | | AVARYCDLPSKLGHKLN* |
| Bcl-xL- | 289 | 22d, 25b | MASNFTQFVLVDNGGTGDVTVAPSNFANGIAEWISSNSR |
| TagBFP | | “BAD(G)” | SQAYKVTCSVRQSSAQNRKYTIKVEVPKGAWRSYLNMEL |
| MCP-linker- | | | TIPIFATNSDCELIVKAMQGLLKDGNPIPSAIAANSGIY |
| ADAR2- | | | GGSGSGAGSGSPAGGGAPGSGGGSQLHLPQVLADAVSRL |
| DDN- | | | VLGKFGDLTDNFSSPHARRKVLAGVVMTTGTDVKDAKVI |
| Bad(F)- | | | SVSTGTKCINGEYMSDRGLALNDCHAEIISRRSLLRFLYT |
| ADAR2(E4 | | | QLELYLNNKDDQKRSIFQKSERGGFRLKENVQFHLYISTS |
| 88Q)-DDC- | | | PCGDARIFSPHEPILEEPAASGSGTGAPPNLWAAQRYGRE |
| Bcl-xL- | | | LRRMSDEGVDRHPNRKARGQLRTKIESGQGTIPVRSNASI |
| TagBFP | | | QTWDGVLQGERLLTMSCSDKIARWNVVGIQGSLLSIFVE |
| | | | PIYFSSIILGSLYHGDHLSRAMYQRISNIEDLPPLYTLNK |
| | | | PLLSGISNAEARQPGKAPNFSVNWTVGDSAIEVINATTGK |
| | | | DELGRASRLCKHALYCRWMRVHGKVPSHLLRSKITKPNVY |
| | | | HESKLAAKEYQAAKARLFTAFIKAGLGAWVEKPTEQDQF |
| | | | SLTGSAAGGSGGSAAASSNRELVVDFLSYKLSQKGYSWS |
| | | | QFSDVEENRTEAPEGTESEMETPSAINGNPSWHLADSPA |
| | | | VNGATGHSSSLDAREVIPMAAVKQALREAGDEFELRYRR |
| | | | AFSDLTSQLHITPGTAYQSFEQVVNELFRDGVNWGRIVA |
| | | | FFSFGGALCVESVDKEMQVLVSRIAAWMATYLNDHLEPW |
| | | | IQENGGWDTFVELYGNNGSSELIKENMHMKLYMEGTVDN |
| | | | HHFKCTSEGEGKPYEGTQTMRIKVVEGGPLPFAFDILAT |
| | | | SFLYGSKTFINHTQGIPDFFKQSFPEGFTWERVTTYEDGG |
| | | | VLTATQDTSLQDGCLIYNVKIRGVNFTSNGPVMQKKTLG |
| | | | WEAFTETLYPADGGLEGRNDMALKLVGGSHLIANIKTTY |
| | | | RSKKPAKNLKMPGVYYVDYRLERIKEANNETYVEQHEV |
| | | | AVARYCDLPSKLGHKLN* |
| MCP-linker- | 290 | 23b “WT” | MASNFTQFVLVDNGGTGDVTVAPSNFANGIAEWISSNSR |
| ADAR2- | | Note | SQAYKVTCSVRQSSAQNRKYTIKVEVPKGAWRSYLNMEL |
| DDN- | | Other | TIPIFATNSDCELIVKAMQGLLKDGNPIPSAIAANSGIY |
| MS1(A)- | | mutants are | GGSGSGAGSGSPAGGGAPGSGGGSQLHLPQVLADAVSRL |
| ADAR2(E4 | | bolded | VLGKFGDLTDNFSSPHARRKVLAGVVMTTGTDVKDAKVI |
| 88Q)-DDC- | | 25b | SVSTGTKCINGEYMSDRGLALNDCHAEIISRRSLLRFLYT |
| TagBFP | | | QLELYLNNKDDQKRSIFQKSERGGFRLKENVQFHLYISTS |
| | | | PCGDARIFSPHEPILEEPAASGGSGGSGRPEIWMTQGLRR |
| | | | LGDEANAYYARRTGDRHPNRKARGQLRTKIESGQGTIPVR |
| | | | SNASIQTWDGVLQGERLLTMSCSDKIARWNVVGIQGSLLS |
| | | | IFVEPIYFSSIILGSLYHGDHLSRAMYQRISNIEDLPPLY |
| | | | TLNKPLLSGISNAEARQPGKAPNFSVNWTVGDSAIEVINA |
| | | | TTGKDELGRASRLCKHALYCRWMRVHGKVPSHLLRSKITK |
| | | | PNVYHESKLAAKEYQAAKARLFTAFIKAGLGAWVEKPTEQ |
| | | | DQFSLTGSGSSELIKENMHMKLYMEGTVDNHHFKCTSEGE |
| | | | GKPYEGTQTMRIKVVEGGPLPFAFDILATSFLYGSKTFI |
| | | | NHTQGIPDFFKQSFPEGFTWERVTTYEDGGVLTATQDTS |
| | | | LQDGCLIYNVKIRGVNFTSNGPVMQKKTLGWEAFTETL |
| | | | YPADGGLEGRNDMALKLVGGSHLIANIKTTYRSKKPAKN |
| | | | LKMPGVYYVDYRLERIKEANNETYVEQHEVAVARYCDL |
| | | | PSKLGHKLN* |
| MCP-linker- | 291 | 23c “WT” | MASNFTQFVLVDNGGTGDVTVAPSNFANGIAEWISSNSR |
| ADAR2- | | Note | SQAYKVTCSVRQSSAQNRKYTIKVEVPKGAWRSYLNMEL |
| DDN- | | Other | TIPIFATNSDCELIVKAMQGLLKDGNPIPSAIAANSGIY |
| MS1(G)- | | mutants | GGSGSGAGSGSPAGGGAPGSGGGSQLHLPQVLADAVSRL |
| ADAR2(E4 | | are | VLGKFGDLTDNFSSPHARRKVLAGVVMTTGTDVKDAKVI |
| 88Q)-DDC- | | bolded | SVSTGTKCINGEYMSDRGLALNDCHAEIISRRSLLRFLYT |
| TagBFP | | 25c | QLELYLNNKDDQKRSIFQKSERGGFRLKENVQFHLYISTS |
| | | | PCGDARIFSPHEPILEEPAASGGSGGSGRPEIWMTQGLRR |
| | | | LGDEGNAYYARRTGDRHPNRKARGQLRTKIESGQGTIPVR |
| | | | SNASIQTWDGVLQGERLLTMSCSDKIARWNVVGIQGSLLS |
| | | | IFVEPIYFSSIILGSLYHGDHLSRAMYQRISNIEDLPPLY |
| | | | TLNKPLLSGISNAEARQPGKAPNFSVNWTVGDSAIEVINA |
| | | | TTGKDELGRASRLCKHALYCRWMRVHGKVPSHLLRSKITK |
| | | | PNVYHESKLAAKEYQAAKARLFTAFIKAGLGAWVEKPT |
| | | | EQDQFSLTGSGSSELIKENMHMKLYMEGTVDNHHFKCTS |
| | | | EGEGKPYEGTQTMRIKVVEGGPLPFAFDILATSFLYGSKT |
| | | | FINHTQGIPDFFKQSFPEGFTWERVTTYEDGGVLTATQDT |
| | | | SLQDGCLIYNVKIRGVNFTSNGPVMQKKTLGWEAFTETL |
| | | | YPADGGLEGRNDMALKLVGGSHLIANIKTTYRSKKPAKN |
| | | | LKMPGVYYVDYRLERIKEANNETYVEQHEVAVARYCDL |
| | | | PSKLGHKLN* |

### Example 13: mRNA Variants

This section details different mRNA compositions that can be coupled with the iADAR technology to produce functional mRNA circuits that modulate the translation or function of a gene of interest. Prior compositions demonstrated the ADAR-based editing of a UAG-STOP codon contained within a hairpin to UIG, which is interpreted by the ribosome as the UGG-tryptophan codon. By encoding a unique RNA motif and a gene of interest downstream of the editable stop codon, translation of the gene of interest can be controlled with ADAR and iADAR constructs. The iADAR platform can be used to control any transcript that can accommodate a dsRNA loop and is sensitive to an A to I event. Additionally, using multiple editable sites on a single transcript can permit multi-input logic.

To expand the scope of the technology beyond single-input STOP codon editing, ADAR-sensitive reporters that function via distinct mechanisms were constructed and tested.

### START-Codon Editing

### Creation of a START Codon (AUA to AUI)

AUI Start Codon Changes Initiation (out of frame to in-frame)

In most of biology, an AUG START codon is necessary for the initiation of proper translation, though there are exceptions. As inosine is typically interpreted by the cell as a guanine, it was tested whether an ADAR-generated AUI could serve as an initiation site for translation when in the 5′UTR of a gene of interest (see e.g., FIG. 26a). With this, it would be possible to generate novel initiation sites of translation to control a gene of interest, where the novel start codon leads to in-frame translation of the full gene.

To test this, an ADAR substrate was designed composed of a dsRNA hairpin containing both an AUA-codon with a mismatch at the second adenine to promote specific deamination to AUI (as opposed to IUA or IUI) and an MS2 operator motif (see e.g., FIG. 26b). The design of one large dsRNA hairpin was chosen to promote correct folding of RNA secondary structures. Previous studies have shown that MS2 operators in the 5′UTR of eukaryotic mRNA can lead to decreased translational efficiency when co-expressed with its corresponding coat protein (MS2 coat protein, or MCP), so high and low affinity variants of the MS2 operator (called here MS2-C and MS2-A respectively for the base at the stem loop) were tested with the idea that the lower affinity MS2-A loop would be less likely to inhibit translation by coat protein alone. As positive controls, the same loops were also designed with the correct AUG start codon.

These designs were cloned into pcDNA3 vectors containing a bidirectional CMV promoter driving constitutive dTomato and EGFPd2 (see e.g., FIG. 26c). The 5′UTR and start codon of EGFPd2 were replaced with the editable start codon designs so the translational efficiency could be measured by looking at the EGFPd2 fluorescence relative to dTomato when co-expressed with MCP-ADAR. In this construct, if the correct start codon is skipped, the next AUG codon leads to a short, out of frame translation product.

HEK293FT cells were co-transfected with each of the reporter constructs (MS2-C w/ AUG start, MS2-C w/ AUA start, MS2-A w/ AUG start, and MS2-A w/ AUA start) and either filler DNA, inactivated ADAR2-DD(E396A) fused to MCP (MCP-dADAR), or active ADAR2-DD(E488Q) fused to MCP (MCP-ADAR). 48 hours post-transfection, cells were lifted and analyzed by flow cytometry. Transfected cells were gated by dTomato, and the median EGFPd2-to-dTomato ratio for the population was calculated per replicate (n=3) by FLOWJO. 2-Way ANOVA was performed to determine significance.

Co-transfection of the MS2-C reporters with MCP-dADAR and MCP-ADAR significantly decreased the relative fluorescence compared to transfecting with filler DNA (NT) (see e.g., FIG. 26d). While there was some increase of the relative fluorescence when co-transfecting the AUA reporter and the active MCP-ADAR, this increase was not significant relative to the non-cotransfected (NT) condition (see e.g., FIG. 26d).

Comparatively, co-transfection of MCP-dADAR and ADAR with the weaker affinity MS2-A substrate had a more modest negative effect on translational efficiency from a native AUG start codon. More importantly, co-transfection of the MS2-A w/ AUA substrate with MCP-ADAR led to a significant increase in the relative EGFPd2/dTomato fluorescence.

This data indicates that MCP-ADAR2-DD fusion proteins are capable of increasing the rate of translation initiation by converting some fraction of an initiation-deficient AUA codon in the 5′UTR to AUI.

### Editing of an AUA Codon to Create an Upstream, In-frame Motif

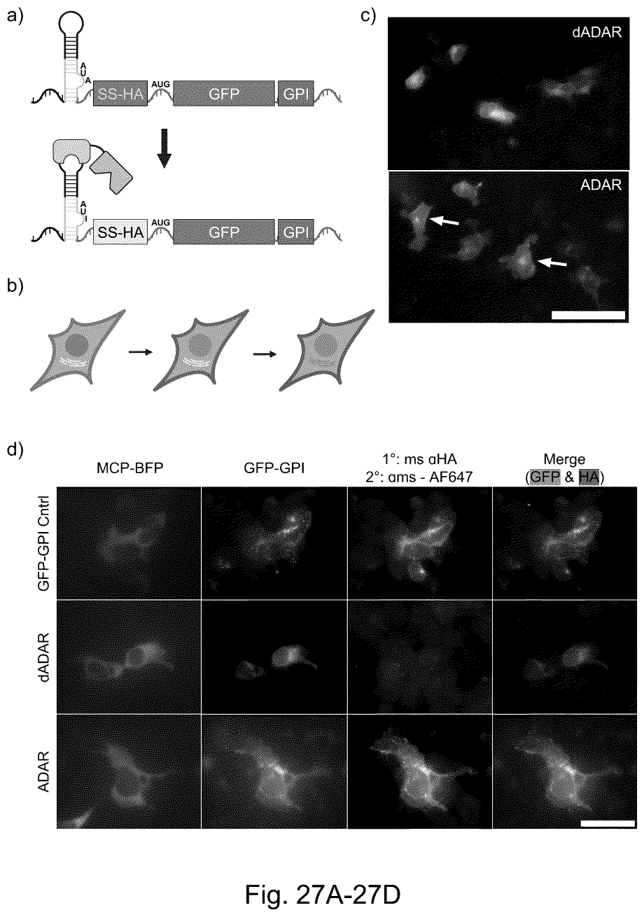

The previous schemes demonstrated the capability to turn on or off translation by editing the START codon, as the subsequent AUG codon in those constructs were out of frame with the EGFPd2. Subsequent tests focused on whether translation initiation could be shifted to another START codon that is in frame with the original START codon (see e.g., FIG. 27a). In this way, a polypeptide sequence can be added to the original translation product that would modify the activity or localization of the original translation product.

To test this, a reporter construct was designed that contained: an MS2-A editable AUA start codon upstream of the murine IgGκ signal sequence and HA epitope tag (SS-HA), an in frame AUG start codon, GFP, and the glycosylphosphatidylinositol (GPI) anchor sequence from Thymocyte differentiation antigen 1 (GPI in diagram) (see e.g., FIG. 27a). With this reporter, in the absence of editing, the majority of the translated product will be from the AUG codon directly upstream of GFP. This will lead to the translation product residing in the cytoplasm with a non-functional GPI anchor (as this post-translational modification (PTM) must occur in the endoplasmic reticulum (ER) lumen). Upon ADAR editing of the upstream AUA to AUI, translation will initiate upstream of the signal sequence, leading to co-translational translocation of HA-GFP-GPI into the ER lumen, the addition of the GPI anchor PTM, and presentation of HA-GFP on the plasma membrane (see e.g., FIG. 27b). With this construct, the editing can be monitored by either by microscopy (looking for ER & plasma membrane localization) or directly by immunostaining for the HA-tag.

HEK cells were co-transfected with the GFP-GPI reporter and either filler DNA, MCP-dADAR, or MCP-ADAR. A GFP-GPI with a functional start codon before the secretion signal was also transfected as a positive control (GFP-GPI Control). One day after transfection, HEK cells were imaged live, and then fixed and stained for HA tag with a primary anti-HA antibody and a secondary antibody that was conjugated to ALEXAFLUOR 647 (AF647).

When imaging live cells, the subcellular GFP expression pattern for the AUA-START construct was found to be dependent on co-transfection of active MCP-ADAR (see e.g., FIG. 27c). The cellular distribution of GFP of the SS-GFP-GPI positive control and the AUA-SS-GFP-GPI co-transfected with MCP-ADAR looked very similar, whereas the AUA-SS-GFP-GPI construct co-transfected with inactive MCP-dADAR led to a different, more cytoplasmic distribution (see e.g., FIG. 27c). After imaging live, cells were fixed with paraformaldehyde, permeabilized using a light detergent, and stained for HA tag. With this, robust staining was observed for the GFP-GPI control and for AUA vectors co-transfected with active MCP-ADAR (see e.g., FIG. 27d). However, there was not much staining for cells co-transfected with the AUA construct and catalytically inactivated MCP-dADAR.

This data demonstrates that the AUA to AUI creation of a start codon can be used to add short motifs to existing proteins.

### Destruction of an AUG Start Codon to JUG

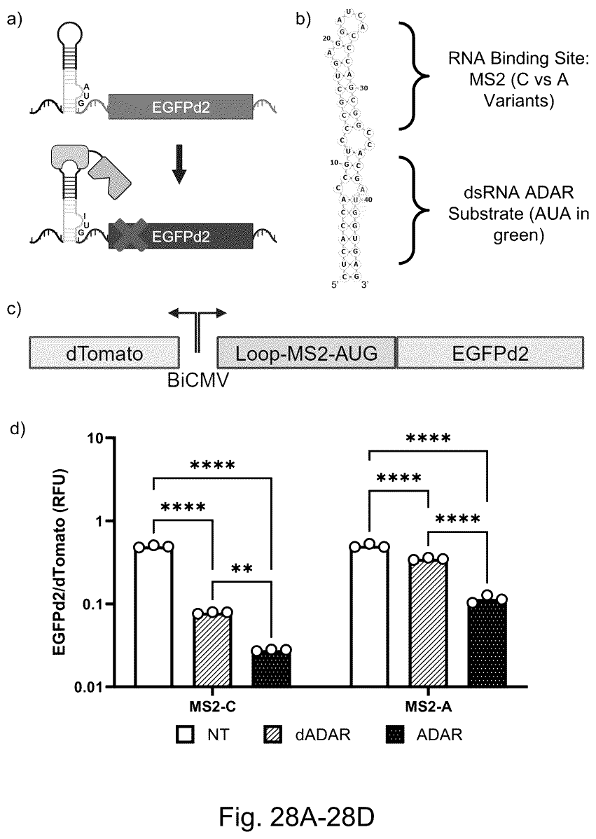

The inverse of the above scheme would be the destruction of an AUG start codon by deamination of the adenosine to IUG (see e.g., FIG. 28a). This edited codon would be most likely skipped by the pre-initiation complex (if read as GUG), leading to a decrease in the translation efficiency of a gene of interest. This can be used to make an “OFF” switch when there is no alternative start that leads to productive translation.

To test this, an ADAR substrate was designed similar to the substrates tested above, but with an AUG codon in a dsRNA hairpin with a mismatch to promote the deamination of the correct adenine (see e.g., FIG. 28b). This scheme was similarly tested with both the high affinity MS2-C and lower affinity MS2-A variants by cloning these substrates into a pcDNA3 vector with a bidirectional CMV driving the expression of dTomato and EGFPd2 (see e.g., FIG. 28c).

HEK cells were co-transfected with the two different reporter constructs (MS2-C and MS2-A) and either filler DNA, MCP-dADAR, or MCP-ADAR. 48 hours post-transfection, cells were lifted and analyzed by flow cytometry. Transfected cells were gated by dTomato, and the median EGFPd2-to-dTomato ratio for the population was calculated per replicate (n=3) by FLOWJO. 2-Way ANOVA was performed to determine significance.

Co-transfection of the high affinity MS2-C based construct with either MCP fusion protein (active or inactive ADAR) led to a significant decrease in translational efficiency of EGFPd2 (see e.g., FIG. 28d). Additionally, there was a further significant difference between the active and inactive ADAR variants, indicating that the translational repression due to the protein-RNA interaction was further enhanced by the active ADAR (e.g., through deamination of the correct start codon). Similar to MS2-C, the MS2-A based construct also had a significant decrease in translational efficiency for both MCP-dADAR and MCP-ADAR (see e.g., FIG. 28d). Co-transfection with the active ADAR variant led to a further, significant decrease in relative EGFPd2 levels in the cells.

These experiments demonstrate that a start codon can be edited to increase or decrease expression of a gene of interest from the ADAR platform.

### Sense Codon Editing

In addition to controlling gene activity by editing the initiation or termination of translation, this iADAR technology can be used to recode a protein by deamination of in-frame codons (e.g., to introduce mutations to specific sites). In order to do this, one would need to introduce a dsRNA loop and an RNA binding motif to an adjacent codon of interest (see e.g., FIG. 29a). The expression of active ADAR would lead to deamination of an adenine base to inosine, which would be interpreted as guanine and a different amino acid would be inserted into the growing polypeptide chain. The number of possible recoding permutations is constrained by the genetic code, e.g., in human cells, it is impossible to use adenosine deamination to convert from or to a proline, as the four proline codons are CCN (see e.g., Table 15 for possible functional recoding). However, there are still scenarios where this functionality may be desirable.

To show inducible ADAR recoding of a protein's amino acid composition, a construct was designed that contained: a CD8 signal peptide, an HA epitope tag, the self-labeling HaloTag, a mutant variant of the thosea asigna virus 2A self-cleaving peptide that is defective for ribosome skipping (Gly18Arg mutation), the DNA binding domain of Gal4, and a 4× repeat of the transcriptional activation domain from herpes virus (VP64) (see e.g., FIG. 29a). The penultimate glycine in 2A peptides is invariant (see e.g., FIG. 29b) and any mutation leads to defective ribosome skipping. The codon for the G18R residue (AGG) is in a dsRNA hairpin with the high affinity MS2-C motif, which acts as a substrate for targeted MCP-ADAR. In the absence of ADAR editing, the defective T2A sequence will lead to the topological sequestering of the Gal4-VP64 transcription factor in the ER lumen due to the fusion to the secreted HaloTag (see e.g., FIG. 29c). Upon ADAR editing of the Arg18 codon to glycine (AGG to IGG), the T2A regains the function of ribosome skipping. This leads to the protein domains downstream of the T2A sequence to be translated as a separate polypeptide in the cytoplasm, whereby the GAL4-VP64 transcription factor will translocate to the nucleus and turn on the expression of a fluorescent H2B-mCherry reporter (see e.g., FIG. 29c). This demonstrates the ability of directed ADAR domains to edit and recode a protein sequence of interest to modulate protein function.

To test this, an in-house clonal HEK293FT cell line that contains an integrated UAS-H2B-mCherry reporter was co-transfected with the T2A(G18R) construct and either filler DNA, MCP-dADAR, or MCP-ADAR. A GAL4-VP64 positive control was also transfected into HEK cells. One day post transfection, cells were imaged via microscopy to check for transcriptional activation via H2B-mCherry expression (see e.g., Example 16).

### Two-Input Logic

### Two-Input AND-Gate by Using Multiple STOP Codons and RNA Motifs

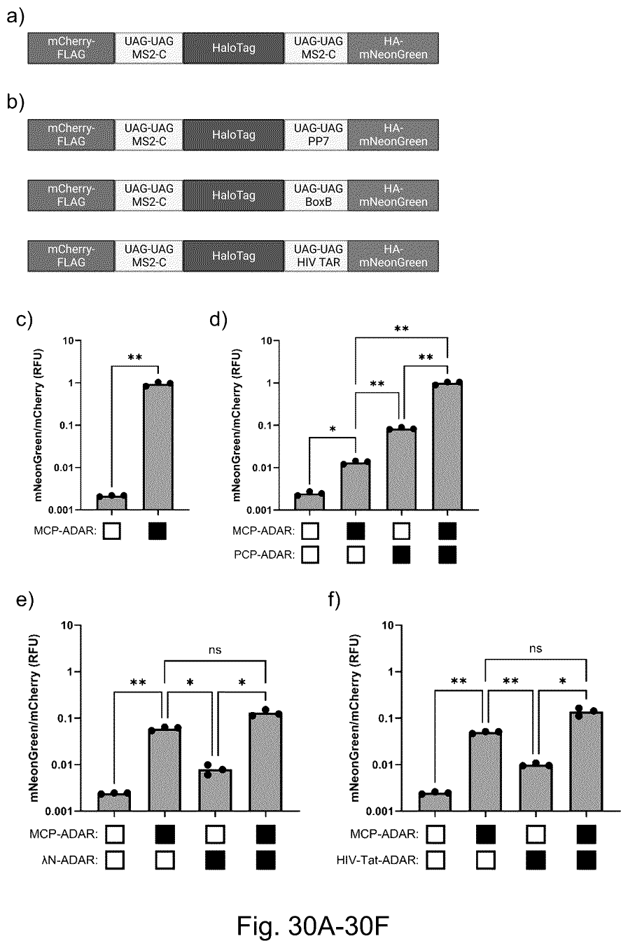

In other reporter designs, adding additional STOP codons and RNA binding motifs (RBM) was found to lead to reduced background of the system without severely affecting the maximum editing efficiency (e.g., mCherry-2×STOP-MS2-HaloTag-2×STOP-MS2-mNeonGreen) (see e.g., FIG. 30a). In addition, the RBM of the reporter and the RNA binding domain (RBD) fused to ADAR2-DD can be swapped to get a level of orthogonal activation by editing.

Multi-input logic gates can be constructed from different RBMs and RBDs and used in series on a single transcript (see e.g., FIG. 30b). The expression of one active RBD-ADAR fusion construct alone can lead to minimal editing of all 4 STOP codons, and expression of both RBD fusion proteins can allow get full editing and expression of the downstream gene. This can then be applied to make multi-input iADAR circuits on a single mRNA.

To test this, three reporters were constructed that were composed of an upstream mCherry-FLAG-P2A-T2A, 2×UAG stop codon loop beside an MS2 RBM, a HaloTag-T7 tag spacer, another 2×UAG stop codon loop with a different RBM (PP7, BoxB or HIV TAR), and a downstream HA-mNeonGreen(M10K) reporter (see e.g., FIG. 30b). 75,000 HEK293FT cells were then co-transfected with 50 ng of reporter DNA and either 30 ng of filler DNA, 15 ng of MCP-ADAR and 15 ng of filler DNA, 15 ng of the respective RBD-ADAR fusion (PCP, λN, or HIV-Tat) and 15 ng of filler DNA, or 15 ng of both MCP-ADAR and RBD-ADAR. 48 hours post-transfection, cells were analyzed by flow cytometry, gated for mCherry expression, and the median mNeonGreen to mCherry value per well was computed. One-way ANOVA statistical analysis was performed (n=3).

When compared to the reporter consisting of two MS2-C loops (see e.g., FIG. 30c), the background and maximal activation of the dual-input MS2 and PP7 reporter was very similar (see e.g., FIG. 30d). Co-transfection of both MCP-ADAR and PCP-ADAR led to significantly higher translational efficiency compared to cells that were singly transfected with MCP-ADAR or PCP-ADAR alone (see e.g., FIG. 30d). However, the same result was not observed for the dual-input reporters consisting of MS2 & BoxB (see e.g., FIG. 30e) or MS2 & HIV-TAR (see e.g., FIG. 30f).

This data demonstrates the capability of the system to be extended by encoding multiple ADAR substrates and RBMs on a single transcript.

### STOP-Codon Insertion Between Functional Domains

In other iterations of the STOP codon editing iADAR reporters, the STOP codons were positioned between two polypeptides that were separated by self-cleaving 2A peptides, and functioned as distinct components. As an example, the reporter shown in FIG. 30a has three separate protein domains (mCherry, HaloTag, and mNeonGreen) that all operate independently of each other.

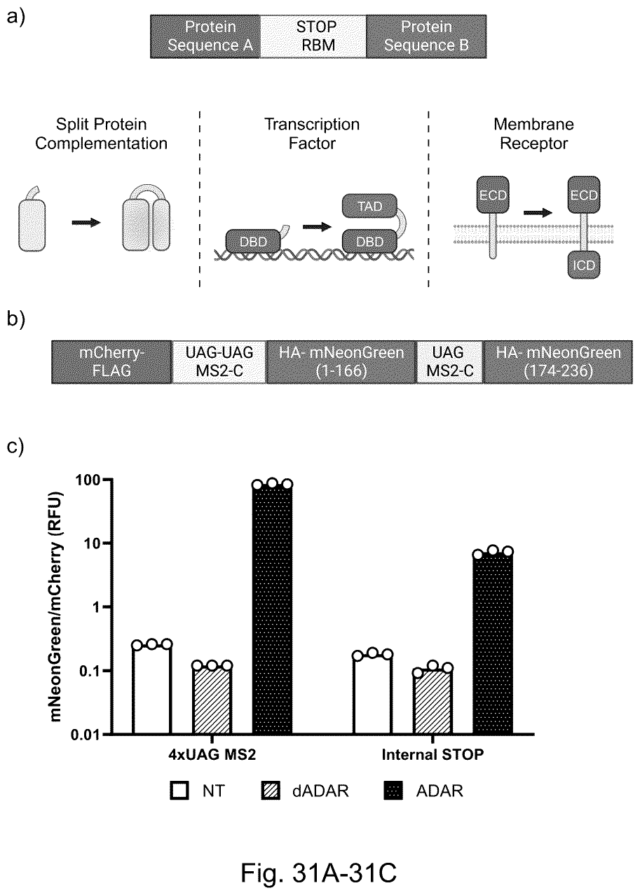

In certain circumstances, it can be advantageous to have the recoding of a STOP codon to a tryptophan codon lead to the creation of a new fusion protein, where the upstream and downstream domains are fused together as a single, functional polypeptide chain (see e.g., FIG. 31a). Examples where this can be useful include split proteins of interest (N-terminal domain and C-terminal domain), transcription factors (DNA-binding domain (DBD) and transcriptional activation domain (TAD)), membrane receptors (extracellular domain, transmembrane domain and intracellular domain), or any multi-domain protein (see e.g., FIG. 31a). One advantage of this approach can be the ability to modulate protein effectors more sharply by having the pre-edited translation product have an opposing effect to the post-edited product. In the case of transcription factors, expressing the pre-edited DBD can act to repress transcription initially, and the post-edited DBD-TAD fusion protein will lead to transcription (see e.g., FIG. 31a). This is one example, but this can be a generally desirable feature.

To demonstrate the capability of the iADAR platform to create functional fusion proteins post-editing, a reporter was designed and tested that contained an MS2-directed editable STOP codons between 2A peptides and in the middle of mNeonGreen (see e.g., FIG. 31b). The STOP codon loop inserted into mNeonGreen was designed adjacent to a flexible loop, and all amino acids outside of the loop were retained (other than the W167 position which was mutated to a STOP codon). In the case of this reporter, mNeonGreen fluorescence only occurs when editing of both sets of STOP codons occurs. This would therefore serve as a demonstration of STOP codon editing leading to a functional fusion protein.

To test this, 70,000 HEK293FT cells were co-transfected with 50 ng of either the 2×UAG-Internal-STOP-MS2 reporter or the previously tested 4×UAG-MS2 reporter plasmids, and 30 ng of either non-ADAR encoding DNA (NT), inactive MCP-dADAR (dADAR), and active MCP-ADAR (ADAR) plasmids. 48 hours post-transfection, cells were lifted and analyzed by flow cytometry, gated for mCherry expression, and the median mNeonGreen to mCherry value per well was computed. n=3 transfections.

The insertion of the editable STOP codon and MS2 loop into the coding sequence for mNeonGreen (Internal STOP) led to editing dependent mNeonGreen expression (see e.g., FIG. 31c). Compared to the original 4×UAG-MS2 reporter, the induced fold change was reduced, but the background fluorescence was also lower.

Although the construct was initially designed to determine if there was a more sensitive iADAR reporter, this data demonstrates iADAR mRNA variants where the activity of the payload is dependent on the fusion of the pre-STOP and post-STOP components.

### Example 14: iADAR Protein Variants

This section details new or enhanced designs of the protein components of the iADAR platform (i.e. the engineering of the enzymatic domain of adenosine deaminase).

### Inclusion of ADAR Mutations and Localization Domain for Antigen Sensing

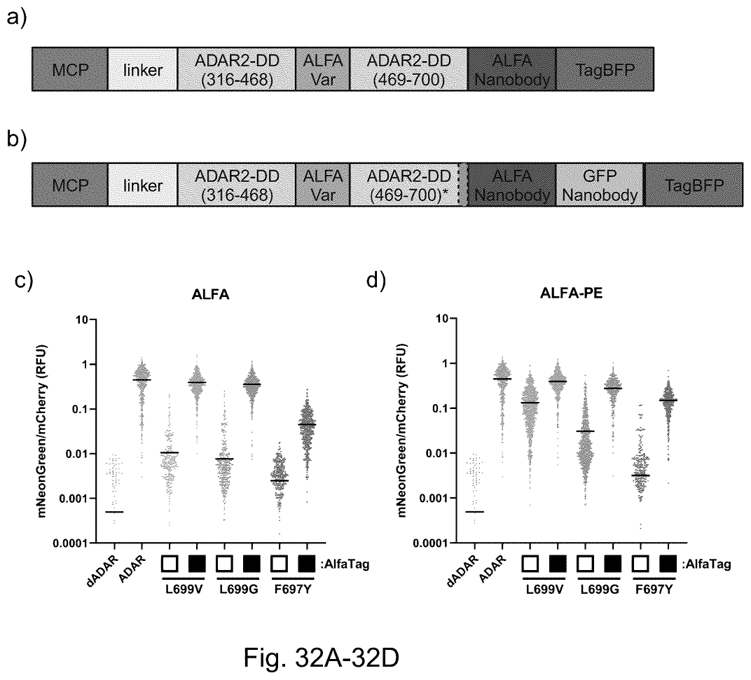

The AlfaTag epitope can be sensed by an AlfaTag inducible ADAR domain which contains: MS2 coat protein, N-ADAR2-DD(316-468), AlfaTag epitope or epitope variants, C-ADAR2-DD(469-700), and Alfa-Nanobody (AlfaNb) (see e.g., FIG. 32a). Lower affinity epitope variants (AlfaPE & Alfa78) can be used, however they had increased background activity independent of soluble AlfaTag expression. Introducing certain mutations into the ADAR2-DD led to decreased background and an increase in fold change in drug inducible iADAR systems (Bcl-xL and Mcl-1). The same mutations were introduced into the weaker AlfaTag epitope variants to reduce the background of the system.

To test this, constructs were created that were similar to those before but with the addition of the mutations to the ADAR2-DD (L699V, L699G & F697Y) and an additional single-domain antibody fragment capable of binding to GFP and its derivatives (VHH9) (see e.g., FIG. 32b). In addition, non-fluorescent EGFP(R96M) and EGFP(R96M)-AlfaTag fusion proteins were constructed as the control and experimental stimuli respectively. HEK293FT cells were then co-transfected with 4×UAG-MS2 iADAR reporter (see e.g., FIG. 32a), either EGFP(R96M) or EGFP(R96M)-AlfaTag, and the different mutations of the AlfaTag inducible iADAR. 48 hours after transfection, cells were imaged via microscopy and the mNeonGreen-to-mCherry ratio was computed for individual iADAR-BFP-positive cells via ImageJ and the mean was computed by GRAPHPAD PRISM. n=1 replicate of transfected HEK.

For the high-affinity ALFA variant of the iADAR, the addition of stronger mutations did not greatly reduce the mean background (L699V—0.010 RFU, L699G—0.0077 RFU, F697Y—0.0025 RFU) or increase the mean fold change of the uninduced to induced system (L699V—37×, L699G—46×, F697Y—18×) (see e.g., FIG. 32c). However, the insertion of the weaker variant, ALFA-PE, into the iADAR system did greatly affect the mean background level (L699V—0.13 RFU, L699G—0.030 RFU, F697Y—0.0031 RFU) and the mean fold change of the system (L699V—3.0×, L699G—9.1×, F697Y—47×) (see e.g., FIG. 32d).

This data demonstrates that the addition of mutations decreases the background and increases the fold-change for the lower-affinity nanobody-epitope pairs. While ALFA-AlfaNb interaction is reported to be picomolar, the ALFA-PE-AlfaNb interaction is reported to be nanomolar, which is similar to other intrabody-epitope interaction strengths.

Multi-Input iADAR Proteins

While the dual-input reporter data (Two-Input Logic) indicated that it is possible to construct multi-input logic using multiple RBD-iADAR fusion proteins with a single mRNA transcript, it is advantageous to be able to encode this multi-input logic with a single RBD-iADAR protein sensor. An auto-inhibited ADAR-DD can require two events (e.g., antigen, drug, protease, etc.) to fully relieve the autoinhibited state of a single enzymatic domain (see e.g., FIG. 33a).

In order to test this, a plasmid was constructed that combined Bcl-xL-BAD and AlfaNb-ALFA iADARs into a single protein (see e.g., FIG. 33b). The BAD-peptide was fused to the N-terminus of ADAR2-DD and the ALFA-PE peptide was inserted at the 5′ RNA binding site. On the C-terminus of ADAR2-DD, the AlfaNb and Bcl-xL were fused in tandem. The F697Y mutation was also used to decrease the potential of background.

With this system, iADAR activity only occurs when soluble AlfaTag (purple squiggle) was expressed and the Bcl-xL inhibitor A-1331852 (orange hexagon) was added, as the addition of either alone would not disturb the other intramolecular interaction (see e.g., FIG. 33a).

HEK293FT cells were co-transfected with the dual-input iADAR, the 4×UAG-MS2 reporter, and either EGFP(R96M) or EGFP(R96M)-AlfaTag on day 0. At the time of transfection, 500 nM of A-1331852 or DMSO was added to their respective conditions. 48 hours after transfection, cells were lifted and run via flow cytometry. The median mCherry to mNeonGreen ratio was computed for transfected cells were gated by BFP expression and mCherry expression. The experiment was then analyzed via one-way ANOVA (n=3 transfected cell populations per condition).

The highest median relative fluorescence was achieved when both A-1331852 and EGFP(R96M)-AlfaTag were present (see e.g., FIG. 33c), and was significantly higher than either alone (˜110× and ˜4.6× greater than A-1331852 and AlfaTag respectively). Transfection with AlfaTag and treatment with DMSO was also significantly higher than the negative condition or A-1331852 treatment.

This data demonstrates capability of “daisy-chaining” protein components together under the iADAR platform to create dual-input logic. The utility of an optimized version of this system is that it allows for exogenous control (e.g., through small-molecule drugs) of antigen sensing, which can be used in therapeutic contexts.

### Grazoprevir Inducible Systems Updates

Mutation of NS3-peptide based system

An iADAR was constructed and tested that was induced by approved antiviral drugs that relied on the NS3 HCV-protease and a high affinity peptide (see e.g., Development of Grazoprevir Activated ADAR). This system functioned by the same mechanism as the Bcl-xL and Mcl-1 systems, e.g., drug based displacement of a high-affinity intramolecular interaction (see e.g., FIG. 34a).

HEK293FT cells were co-transfected with the 4×UAG MS2 reporter and either an MCP-ADAR containing a peptide insertion at the 5′ RNA binding site (see e.g., FIG. 34b, Pep), grazoprevir iADAR (Pep-NS3) or a mutant version of the iADAR (Pep-NS3 (K690R)). At the time of transfection, 3 μM of DMSO or grazoprevir was added. 48 hours after transfection, cells were lifted and run via flow cytometry. The median mCherry to mNeonGreen ratio was computed for transfected cells that were gated by BFP expression and mCherry expression. The experiment was then analyzed via two-way ANOVA (n=3 transfected cell populations per condition).

The fold change of the grazoprevir treated iADAR compared to DMSO treated was ˜31× for the WT system and ˜43× for the iADAR K690R (see e.g., FIG. 34c). This further demonstrates the utility of various mutations in developing more sensitive iADAR systems.

Ligand-INducible Connection Based iADAR

Catalytically active NS3 and protease inhibitors can be used to create conditional fusion proteins, termed Ligand-INducible Connections (LINCs). The scheme relies on fusion of the NS3 protease and its corresponding cut site(s) between separate or split domains of a protein that relies on both components to function (see e.g., FIG. 35a).

In the context of iADAR, the LiNC system was used to create a grazoprevir inducible ADAR by inserting the NS5A/5B cutsite in the 5′ RNA binding loop (see e.g., FIG. 35b, green). When active NS3 protease is fused to the C-terminus, cleavage occurs and the two ADAR halves dissociates (see e.g., FIG. 35a). When the protease inhibitor grazoprevir is added or a catalytically inactive NS3 (dNS3) is used, the halves remain intact and the iADAR remains active.

To test this, LiNC-ADAR constructs were created with WT, L699G, or F697Y mutations (see e.g., FIG. 35b) and HEK293FT cells were co-transfected with them and the 4×UAG-MS2 reporter in the presence of 2 μM grazoprevir or DMSO vehicle. 48 hours after transfection and drug treatment, cells were lifted and analyzed by flow cytometry. The median mCherry to mNeonGreen ratio was computed for transfected cells that were gated by BFP expression and mCherry expression. (n=3 transfected cell populations per condition).

While the WT LiNC had only a modest drug dependent increase in ADAR editing activity, the L699G mutant had better drug dependent performance (˜13× fold increase when grazoprevir is added) (see e.g., FIG. 35c). This data demonstrates the versatility of engineering ADAR and inserting different epitopes in the 5′ RNA binding site.

### Example 15: Combined Transcript and iADAR—IRES Control

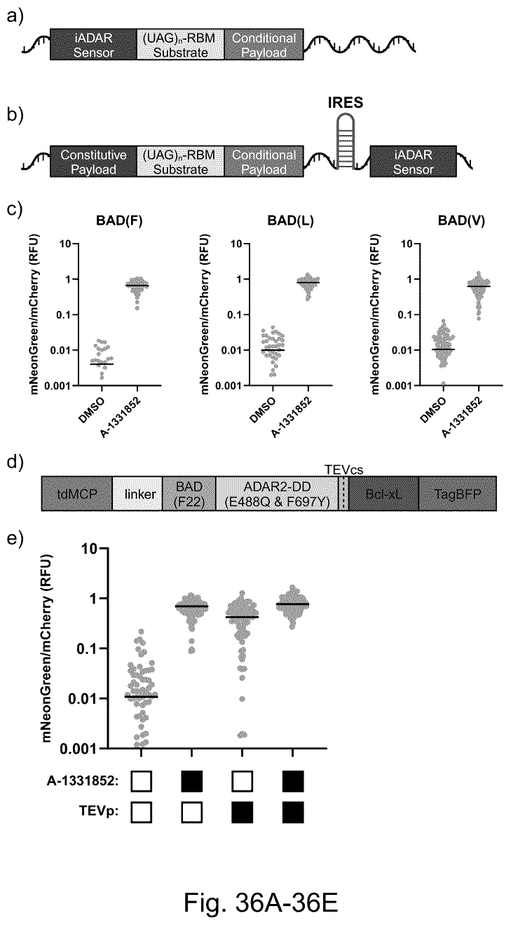

This section details single transcript designs that combine the iADAR-sensor and iADAR-substrate. The iADAR-sensor can be encoded before the first set of editable UAG stop codons to create a single construct capable of sensing and responding to stimuli (see e.g., FIG. 36a). This design is advantageous for linear mRNA therapeutics due to all components of the circuit being deliverable as a single transcript.

As an additional mechanism of achieving the same “all-in-one” design, an mRNA transcript was designed and constructed that encoded both a reporter construct (in this case, the 4×UAG reporter) whose translation is driven by canonical, cap-dependent translation and a downstream iADAR sensor driven by a virally-derived internal ribosome entry site (IRES) (see e.g., FIG. 36b). In this way, a single mRNA, in the absence of the iADAR inducer, would drive the translation of the iADAR sensor and protein of interest (in the case of the data, mCherry). Upon induction, a second, downstream protein (in this case, mNeonGreen) would be translated after stop codon editing. This design can offer the advantage of tunability of iADAR-sensor expression without affecting the expression of the payload after induction. This scheme can also be adaptable to circular mRNA or other modalities that rely on IRES driven expression in cells.

To test this, drug and protease inducible iADAR IRES circuits were constructed. For the drug-inducible iADAR IRES, low background BclXL-BAD pairs were used that contained variant BAD peptides (F22, F22L, & F22V) inserted between AA site 468/469 in the ADAR2-DD and the R522M mutation in the ADAR2-DD domain. The EMCV IRES and the BAD iADARs were placed in the 3′UTR of the 4×UAG reporter construct to create the single construct. 70,000 HEK cells were then transfected with 50 ng of the respective plasmids in the presence of 1 μM A-1331852 or DMSO, and cells were imaged 48 hours later via microscopy. Images were then analyzed by IMAGEJ, where the mNeonGreen-to-mCherry ratio was calculated for individual, transfected cells (gated by mCherry expression). All of the IRES-iADAR constructs were induced by A-1331852 addition relative to DMSO treatment, with fold changes of the mean RFU of 160× for BAD(F22), 80× for BAD(F22L), and 60× for BAD(F22V) (see e.g., FIG. 36c). This showed that this configuration worked as expected, and that using an IRES to drive the expression of iADAR is a viable strategy.

The construction of a protease-inducible iADAR was also tested with the IRES design. To do this, a TEV-inducible iADAR design was tested, composed of: a tandem-dimer MCP, BAD peptide fused to the N-terminus of ADAR2-DD(E488Q & F697Y), a TEV cut site, Bcl-xL, and TagBFP (see e.g., FIG. 36d). This protein functions as an OR-gate, as it can be induced by either dissociation of the BAD-BclxL by addition of A-1331852 or by TEV-based proteolysis of the linker between the C-terminus of the deaminase domain and Bcl-xL.

70,000 HEK cells were then transfected with 50 ng of the plasmid and 3 ng of either filler DNA or a plasmid encoding TEV protease. Each transfection condition was plated in the presence of 1 μM A-1331852 or DMSO. Cells were imaged 48 hours later via microscopy. Images were then analyzed by IMAGEJ, where the mNeonGreen-to-mCherry ratio was calculated for individual, transfected cells (gated by mCherry expression).

All of the IRES-iADAR construct was induced by either the A-1331852 drug or by TEV protease (see e.g., FIG. 36e). The fold change of the mean RFU was 27× for A-1331852 alone, 17× for TEV protease, and 32× for the dual addition of A-1331852 and TEV protease. This demonstrates both the versatility of the IRES based “all-in-one” scheme and the ability for a single iADAR protein to be activated by different stimuli (OR-gate compared to the previous AND-gate).

### Example 16: Sense Codon Editing

In addition to controlling gene activity by editing the initiation or termination of translation, this iADAR technology can be used to recode a protein by deamination of in-frame codons (i.e. introduce mutations to specific sites). In order to do this, one can introduce a dsRNA loop and an RNA binding motif to an adjacent codon of interest (see e.g., FIG. 37a). The expression of active ADAR leads to deamination of an adenine base to inosine, which would be interpreted as guanine and a different amino acid would be inserted into the growing polypeptide chain. The number of possible recoding permutations is constrained by the genetic code, e.g., in human cells, it is impossible to use adenosine deamination to convert from or to a proline, as the four proline codons are CCN (see e.g., Table 15 for possible functional recoding). However, there are still scenarios where this functionality may be desirable.

To test inducible ADAR recoding of a protein's amino acid composition, a construct was designed that contained: a CD8 signal peptide, an HA epitope tag, the self-labeling HaloTag, a mutant variant of the thosea asigna virus 2A self-cleaving peptide that is defective for ribosome skipping (Gly18Arg mutation), the DNA binding domain of Gal4, and a 4× repeat of the transcriptional activation domain from herpes virus (VP64) (see e.g., FIG. 37a). The penultimate glycine in 2A peptides is invariant (see e.g., FIG. 37b) and any mutation leads to defective ribosome skipping. The codon for the G18R residue (AGG) is in a dsRNA hairpin with the high affinity MS2-C motif, which acts as a substrate for targeted MCP-ADAR. In the absence of ADAR editing, the defective T2A sequence will lead to the topological sequestering of the Gal4-VP64 transcription factor in the ER lumen due to the fusion to the secreted HaloTag (see e.g., FIG. 37c). Upon ADAR editing of the Arg18 codon to glycine (AGG to IGG), the T2A regains the function of ribosome skipping. This leads to the protein domains downstream of the T2A sequence to be translated as a separate polypeptide in the cytoplasm, whereby the GAL4-VP64 transcription factor translocate to the nucleus and turn on the expression of a fluorescent H2B-mCherry reporter (see e.g., FIG. 37c). This demonstrates the ability of directed ADAR domains to edit and recode a protein sequence of interest to modulate protein function.

To test this, an in-house clonal HEK293FT cell line that contains an integrated UAS-H2B-mCherry reporter was co-transfected with the editable T2A (also called T2A*) construct (3 ng, 0.3 ng and 0.03 ng) and either MCP-dADAR or MCP-ADAR (30 ng). A GAL4-VP64 positive control was also transfected into HEK cells. One day post-transfection, cells were lifted and analyzed by flow cytometry. Transfected cells were gated by TagBFP, the median H2B-mCherry fluorescence for the population was calculated and H2B-mCherry positive populations within the transfected cells were determined by the percentage of cells above 0.5% of non-transfected control cells per replicate (n=3) by FLOWJO.

Additionally, the clonal HEK line was co-transfected with 3 ng of the T2A* construct and 30 ng of either filler DNA, MCP-dADAR, or MCP-ADAR. 48 hours later lysates were collected with 1×LDS-PAGE loading buffer (THERMOFISHER) on ice. The samples were sonicated, centrifuged, and boiled at 70° C. for 10 min after the addition of 1×Reducing Agent (THERMOFISHER). The samples were run on a 4%-12% bis-tris polyacrylamide gel at 175V for 45 minutes, and the samples were transferred onto a nitrocellulose membrane using the IBLOT (THERMOFISHER). Samples were probed for FLAG tag (Direct-Blot™ HRP anti-DYKDDDDK Tag Antibody, “DYKDDDDK” is disclosed as SEQ ID NO: 406) and GAPDH-loading control (Direct-Blot™ HRP anti-GAPDH Antibody) respectively.

Co-transfecting 3 ng the editable T2A* with the active MCP-ADAR led to a significant increase in the median H2B-mCherry expression compared to the inactive MCP-dADAR control (see e.g., FIG. 37d). The population of the HEK293FT clonal lines that expressed any H2B-mCherry when co-transfected with 0.3 ng (see e.g., FIG. 37e) or 0.03 ng (see e.g., FIG. 37f) of the T2A* construct was significantly higher when co-transfected with MCP-ADAR compared to dADAR, though the percentage of activated cells were lower than the Gal4-VP64 positive control.

Cells that co-transfected with the T2A* construct and active MCP-ADAR had a reduced staining for the main HaloTag-T2A*-FLAG-Gal4-VP64 fusion product (predicted mass of ˜67 kDa) compared to the control and dADAR conditions (see e.g., FIG. 37g). Additionally, the proportion of FLAG-Gal4-VP64 (predicted mass of ˜30 kDa) relative to the main product was increased (although there appears to still be signal in the other conditions). Finally, additional bands corresponding to potential cleavage products are reduced.

These data combined demonstrate the capability of the iADAR system to lead to the functional recoding of a sense codon in a protein-encoding transcript to lead to a change in protein localization and activity.

### Example 17: Exemplary Sequences

TABLE 15
Functional Re-coding Events

| | Unedited | Recoded |
|---|---|---|
| | Codon and | Codon and |
| | Amino Acid | Amino Acid |
| First Position— | AUH-ILE | IUH-VAL |
| ANN to INN | AUG-MET | IUG-VAL |
| | ACN-THR | GCN-ALA |
| | AAY-ASN | IAY-ASP |
| | AAR-LYS | IAR-GLU |
| | AGY-SER | IGY-GLY |
| | AGR-ARG | IGR-GLY |
| Second Position— | UAY-TYR | UIY-CYS |
| NAN to NIN | UAG-STOP | UIG-TRP |
| | CAY-HIS | CIY-ARG |
| | CAR-GLN | CIR-ARG |
| | AAY-ASN | AIY-SER |
| | AAR-LYS | AIR-ARG |
| | GAY-ASP | GIY-GLY |
| | GAR-GLU | GIR-GLY |
| Third Position— | UGA-STOP | UGI-TRP |
| NNA to NNI | AUA-ILE | AUI-MET |
| First & Second Position — | AAY-ASN | IIY-GLY |
| AAN to IIN | AAR-LYS | IIR-GLY |
| Second & Third Position— | UAA-STOP | UII-TRP |
| NAA to NII | | |

*Bold are the recoding events tested herein. Without wishing to be bound by theory, it is hypothesized that any of the above Unedited Codons can be edited into the corresponding Recoded Codon by the iAD polypeptides described herein.

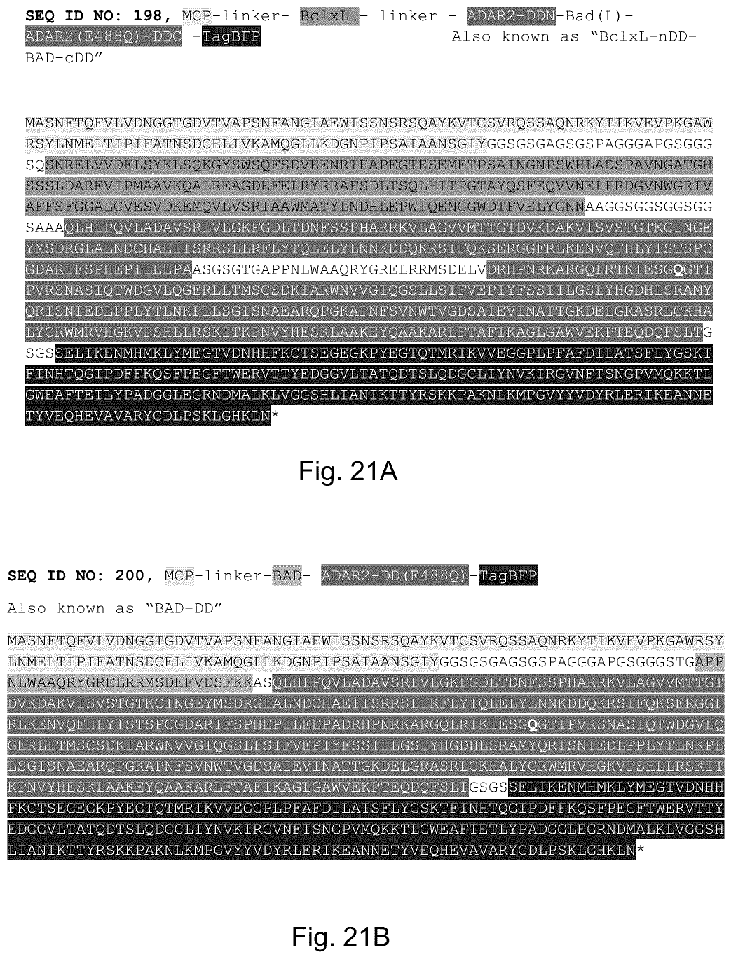

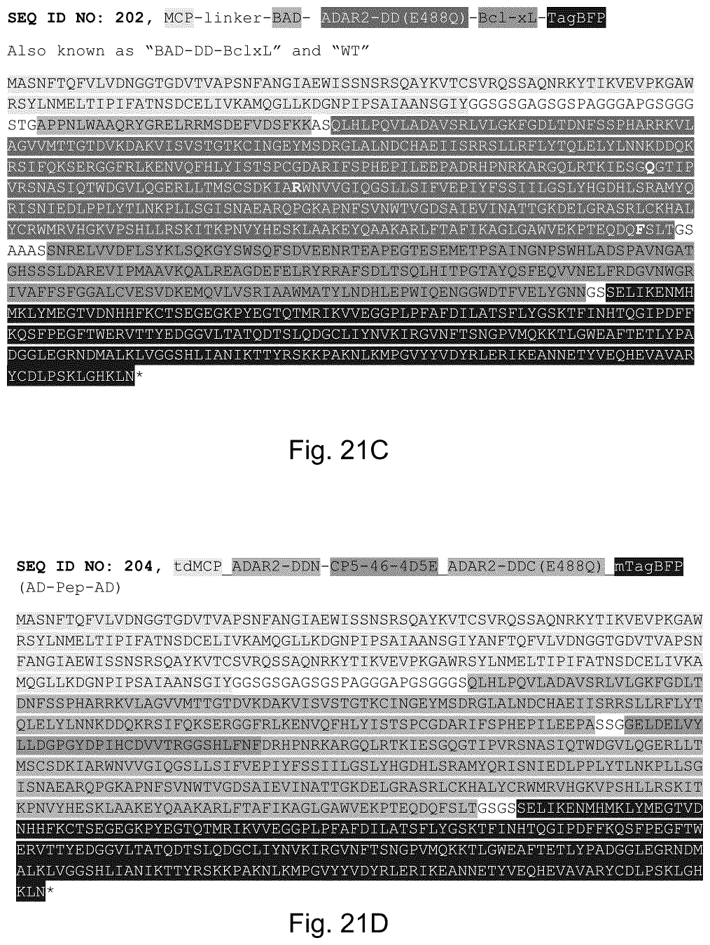

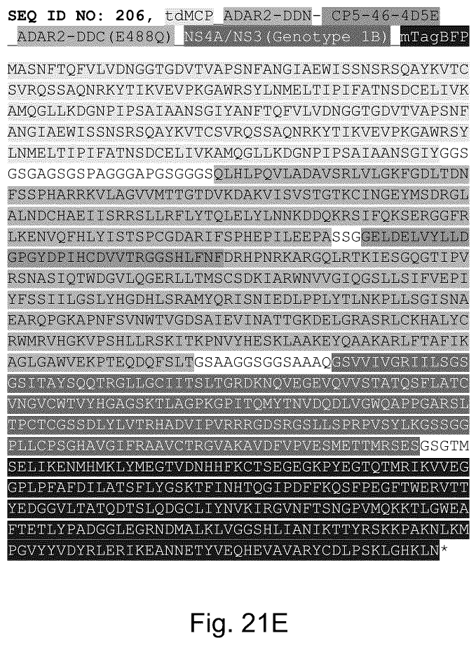

Exemplary sequences (see e.g., FIG. 21A-21E).

SEQ ID NO: 198, MCP-linker-BclxL-
linker-ADAR2-DDN-Bad(L)-ADAR2(E488Q)-
DDC-TagBFP Also known as “BclxL-nDD-BAD-cDD”
(see e.g., FIG. 21A)
MASNFTQFVLVDNGGTGDVTVAPSNFANGIAEWISSNSRSQAYKV
TCSVRQSSAQNRKYTIKVEVPKGAWRSYLNMELTIPIFATNSDCE
LIVKAMQGLLKDGNPIPSAIAANSGIYGGSGSGAGSGSPAGGGAP
GSGGGSQSNRELVVDFLSYKLSQKGYSWSQFSDVEENRTEAPEGT
ESEMETPSAINGNPSWHLADSPAVNGATGHSSSLDAREVIPMAAV
KQALREAGDEFELRYRRAFSDLTSQLHITPGTAYQSFEQVVNELF
RDGVNWGRIVAFFSFGGALCVESVDKEMQVLVSRIAAWMATYLND
HLEPWIQENGGWDTFVELYGNNAAGGSGGSGGSGGSAAAQLHLPQ
VLADAVSRLVLGKFGDLTDNFSSPHARRKVLAGVVMTTGTDVKDA
KVISVSTGTKCINGEYMSDRGLALNDCHAEIISRRSLLRFLYTQL
ELYLNNKDDQKRSIFQKSERGGFRLKENVQFHLYISTSPCGDARI
FSPHEPILEEPAASGSGTGAPPNLWAAQRYGRELRRMSDELVDRH
PNRKARGQLRTKIESGQGTIPVRSNASIQTWDGVLQGERLLTMSC
SDKIARWNVVGIQGSLLSIFVEPIYFSSIILGSLYHGDHLSRAMY
QRISNIEDLPPLYTLNKPLLSGISNAEARQPGKAPNFSVNWTVGD
SAIEVINATTGKDELGRASRLCKHALYCRWMRVHGKVPSHLLRSK
ITKPNVYHESKLAAKEYQAAKARLFTAFIKAGLGAWVEKPTEQDQ
FSLTGSGSSELIKENMHMKLYMEGTVDNHHFKCTSEGEGKPYEGT
QTMRIKVVEGGPLPFAFDILATSFLYGSKTFINHTQGIPDFFKQS
FPEGFTWERVTTYEDGGVLTATQDTSLQDGCLIYNVKIRGVNFTS
NGPVMQKKTLGWEAFTETLYPADGGLEGRNDMALKLVGGSHLIAN
IKTTYRSKKPAKNLKMPGVYYVDYRLERIKEANNETYVEQHEVAV
ARYCDLPSKLGHKLN*
SEQ ID NO: 199,
TAATACGACTCACTATAGGGAGACCCAAGCTGGctagaggatcga
acccttaaggccaccatggcgtccaatttcactcagtttgtgctg
gttgacaacggcgggaccggggacgttacggtagccccctcaaac
tttgccaacggtatagcggagtggataagcagcaattctaggagt
caagcatacaaagttacatgcagcgtgcgccaatctagcgctcag
aatcgcaagtacaccattaaagtagaggtccccaagggagcctgg
agaagctatcttaacatggagttgaccataccaatcttcgctacc
aactctgactgtgaactcattgtgaaagccatgcaaggtctgctc
aaggatggtaacccaattccgtccgctatcgctgccaactctggg
atttacgggggcagtgggagcggtgcaggatctggtagtccagct
gggggaggagcaccgggtagcggtggggggtctcagAGTAACCGG
GAGCTGGTGGTTGACTTTCTCTCCTACAAGCTTTCCCAGAAAGGA
TACAGCTGGAGTCAGTTTAGTGATGTGGAAGAGAACAGGACTGAG
GCCCCAGAAGGGACTGAATCGGAGATGGAGACCCCCAGTGCCATC
AATGGCAACCCATCCTGGCACCTGGCAGACAGCCCCGCGGTGAAT
GGAGCCACTGGCCACAGCAGCAGTTTGGATGCCCGGGAGGTGATC
CCCATGGCAGCAGTAAAGCAAGCGCTGAGGGAGGCAGGCGACGAG
TTTGAACTGCGGTACCGGCGGGCATTCAGTGACCTGACATCCCAG
CTCCACATCACCCCAGGGACAGCATATCAGAGCTTTGAACAGGTA
GTGAATGAACTCTTCCGGGATGGGGTAAACTGGGGTCGCATTGTG
GCCTTTTTCTCCTTCGGCGGGGCACTGTGCGTGGAAAGCGTAGAC
AAGGAGATGCAGGTATTGGTGAGTCGGATCGCAGCTTGGATGGCC
ACTTACCTGAATGACCACCTAGAGCCTTGGATCCAGGAGAACGGC
GGCTGGGATACTTTTGTGGAACTCTATGGGAACAATGCGGCCGGA
GGTAGCGGCGGAAGCGGTGGCTCTGGAGGCTCAGCGGCCGCTCAA
TTAcacctgccccaggttctcgcagacgccgtatcccgccttgta
ctgggcaagtttggtgatcttactgacaatttttcatctcctcat
gcgaggcggaaagtactcgcaggcgtcgtcatgacgaccggaact
gacgtgaaagacgccaaagtcatctctgtctccacgggcacaaag
tgcataaacggggagtacatgagcgaccgggggctggcactgaat
gattgtcacgctgaaataatatctaggcgatctctgcttagattt
ctctacactcaactcgaattgtaccttaacaacaaagatgaccag
aaacgcagtatatttcagaaatcagaacgcggcggatttcgactt
aaggaaaacgttcagttccacttgtatatcagcacatccccttgc
ggtgacgcccgaatcttttccccgcacgagccgatattggaggag
cccgcgGCTAGCGGGTCGGGCACCGGTGCTCCACCCAATCTCTGG
GCAGCGCAGCGCTACGGCCGTGAGCTCAGAAGGATGTCCGATGAG
CTGGTCGACagacatcctaataggaaggctagaggccaacttcgg
acgaagattgaaagtggccagggtactatcccggtgcggtccaac
gctagtattcaaacgtgggacggagtccttcaaggtgaacggctg
ttgacaatgagctgctcagacaaaatcgcgcgctggaatgtagtg
ggaatccaaggcagcctcttgagcatattcgtagaacccatatat
ttctcatccattattttgggctctctgtatcatggtgaccatctg
tcaagggctatgtaccaacgaatttctaatatcgaggatcttcct
ccactctatacactcaataagcctctcttgtccgggatatcaaac
gctgaggcccgccagccagggaaagctcctaacttcagtgttaac
tggaccgttggtgattctgcgatagaggtcatcaacgccacgaca
ggtaaggatgagctcggtagagcctcacgcctgtgtaaacacgcg
ttgtattgtagatggatgagagtacatgggaaggtcccatctcac
ttgctccgaagcaagatcactaagcctaatgtgtatcatgagtca
aaactcgcggctaaagaataccaggcagccaaagctcgacttttt
acagcttttattaaggcagggctcggggcatgggtcgagaagccg
accgagcaggaccaattctctctgacggggagcggatccAGCGAG
CTGATTAAGGAGAACATGCACATGAAGCTGTACATGGAGGGCACC
GTGGACAACCATCACTTCAAGTGCACATCCGAGGGCGAAGGCAAG
CCCTACGAGGGCACCCAGACCATGAGAATCAAGGTGGTCGAGGGC
GGCCCTCTCCCCTTCGCCTTCGACATCCTGGCTACTAGCTTCCTC
TACGGCAGCAAGACCTTCATCAACCACACCCAGGGCATCCCCGAC
TTCTTCAAGCAGTCCTTCCCTGAGGGCTTCACATGGGAGAGAGTC
ACCACATACGAAGACGGGGGCGTGCTGACCGCTACCCAGGACACC
AGCCTCCAGGACGGCTGCCTCATCTACAACGTCAAGATCAGAGGG
GTGAACTTCACATCCAACGGCCCTGTGATGCAGAAGAAAACACTC
GGCTGGGAGGCCTTCACCGAGACGCTGTACCCCGCTGACGGCGGC
CTGGAAGGCAGAAACGACATGGCCCTGAAGCTCGTGGGCGGGAGC
CATCTGATCGCAAACATCAAGACCACATATAGATCCAAGAAACCC
GCTAAGAACCTCAAGATGCCTGGCGTCTACTATGTGGACTACAGA
CTGGAAAGAATCAAGGAGGCCAACAACGAGACCTACGTCGAGCAG
CACGAGGTGGCAGTGGCCAGATACTGCGACCTCCCTAGCAAACTG
GGGCACAAGCTTAATtAAGGGCCCGTTTAAACCCGCTGATCAGCC
TCGACTGTGCCTTCTA
SEQ ID NO: 200,
MCP-linker-BAD-ADAR2-DD(E488Q)-TagBFP Also
known as “BAD-DD” (see e.g., FIG. 21B)
MASNFTQFVLVDNGGTGDVTVAPSNFANGIAEWISSNSRSQAYKV
TCSVRQSSAQNRKYTIKVEVPKGAWRSYLNMELTIPIFATNSDCE
LIVKAMQGLLKDGNPIPSAIAANSGIYGGSGSGAGSGSPAGGGAP
GSGGGSTGAPPNLWAAQRYGRELRRMSDEFVDSFKKASQLHLPQV
LADAVSRLVLGKFGDLTDNFSSPHARRKVLAGVVMTTGTDVKDAK
VISVSTGTKCINGEYMSDRGLALNDCHAEIISRRSLLRFLYTQLE
LYLNNKDDQKRSIFQKSERGGFRLKENVQFHLYISTSPCGDARIF
SPHEPILEEPADRHPNRKARGQLRTKIESGQGTIPVRSNASIQTW
DGVLQGERLLTMSCSDKIARWNVVGIQGSLLSIFVEPIYFSSIIL
GSLYHGDHLSRAMYQRISNIEDLPPLYTLNKPLLSGISNAEARQP
GKAPNFSVNWTVGDSAIEVINATTGKDELGRASRLCKHALYCRWM
RVHGKVPSHLLRSKITKPNVYHESKLAAKEYQAAKARLFTAFIKA
GLGAWVEKPTEQDQFSLTGSGSSELIKENMHMKLYMEGTVDNHHF
KCTSEGEGKPYEGTQTMRIKVVEGGPLPFAFDILATSFLYGSKTF
INHTQGIPDFFKQSFPEGFTWERVTTYEDGGVLTATQDTSLQDGC
LIYNVKIRGVNFTSNGPVMQKKTLGWEAFTETLYPADGGLEGRND
MALKLVGGSHLIANIKTTYRSKKPAKNLKMPGVYYVDYRLERIKE
ANNETYVEQHEVAVARYCDLPSKLGHKLN*
SEQ ID NO: 201,
TAATACGACTCACTATAGGGAGACCCAAGCTGGctagaggatcga
acccttaaggccaccatggcgtccaatttcactcagtttgtgctg
gttgacaacggcgggaccggggacgttacggtagccccctcaaac
tttgccaacggtatagcggagtggataagcagcaattctaggagt
caagcatacaaagttacatgcagcgtgcgccaatctagcgctcag
aatcgcaagtacaccattaaagtagaggtccccaagggagcctgg
agaagctatcttaacatggagttgaccataccaatcttcgctacc
aactctgactgtgaactcattgtgaaagccatgcaaggtctgctc
aaggatggtaacccaattccgtccgctatcgctgccaactctggg
atttacgggggcagtgggagcggtgcaggatctggtagtccagct
gggggaggagcaccgggtagcggtggggggtctACCGGTGCTCCA
CCCAATCTCTGGGCAGCGCAGCGCTACGGCCGTGAGCTCAGAAGG
ATGTCCGATGAGTTCGTCGATTCCTTCAAAAAGGCTAGCcagctg
cacctgccccaggttctcgcagacgccgtatcccgccttgtactg
ggcaagtttggtgatcttactgacaatttttcatctcctcatgcg
aggcggaaagtactcgcaggcgtcgtcatgacgaccggaactgac
gtgaaagacgccaaagtcatctctgtctccacgggcacaaagtgc
ataaacggggagtacatgagcgaccgggggctggcactgaatgat
tgtcacgctgaaataatatctaggcgatctctgcttagatttctc
tacactcaactcgaattgtaccttaacaacaaagatgaccagaaa
cgcagtatatttcagaaatcagaacgcggcggatttcgacttaag
gaaaacgttcagttccacttgtatatcagcacatccccttgcggt
gacgcccgaatcttttccccgcacgagccgatattggaggagccc
gcggacagacatcctaataggaaggctagaggccaacttcggacg
aagattgaaagtggccagggtactatcccggtgcggtccaacgct
agtattcaaacgtgggacggagtccttcaaggtgaacggctgttg
acaatgagctgctcagacaaaatcgcgcgctggaatgtagtggga
atccaaggcagcctcttgagcatattcgtagaacccatatatttc
tcatccattattttgggctctctgtatcatggtgaccatctgtca
agggctatgtaccaacgaatttctaatatcgaggatcttcctcca
ctctatacactcaataagcctctcttgtccgggatatcaaacgct
gaggcccgccagccagggaaagctcctaacttcagtgttaactgg
accgttggtgattctgcgatagaggtcatcaacgccacgacaggt
aaggatgagctcggtagagcctcacgcctgtgtaaacacgcgttg
tattgtagatggatgagagtacatgggaaggtcccatctcacttg
ctccgaagcaagatcactaagcctaatgtgtatcatgagtcaaaa
ctcgcggctaaagaataccaggcagccaaagctcgactttttaca
gcttttattaaggcagggctcggggcatgggtcgagaagccgacc
gagcaggaccaattctctctgacggggagcggatccAGCGAGCTG
ATTAAGGAGAACATGCACATGAAGCTGTACATGGAGGGCACCGTG
GACAACCATCACTTCAAGTGCACATCCGAGGGCGAAGGCAAGCCC
TACGAGGGCACCCAGACCATGAGAATCAAGGTGGTCGAGGGCGGC
CCTCTCCCCTTCGCCTTCGACATCCTGGCTACTAGCTTCCTCTAC
GGCAGCAAGACCTTCATCAACCACACCCAGGGCATCCCCGACTTC
TTCAAGCAGTCCTTCCCTGAGGGCTTCACATGGGAGAGAGTCACC
ACATACGAAGACGGGGGCGTGCTGACCGCTACCCAGGACACCAGC
CTCCAGGACGGCTGCCTCATCTACAACGTCAAGATCAGAGGGGTG
AACTTCACATCCAACGGCCCTGTGATGCAGAAGAAAACACTCGGC
TGGGAGGCCTTCACCGAGACGCTGTACCCCGCTGACGGCGGCCTG
GAAGGCAGAAACGACATGGCCCTGAAGCTCGTGGGCGGGAGCCAT
CTGATCGCAAACATCAAGACCACATATAGATCCAAGAAACCCGCT
AAGAACCTCAAGATGCCTGGCGTCTACTATGTGGACTACAGACTG
GAAAGAATCAAGGAGGCCAACAACGAGACCTACGTCGAGCAGCAC
GAGGTGGCAGTGGCCAGATACTGCGACCTCCCTAGCAAACTGGGG
CACAAGCTTAATtAAGGGCCCGTTTAAACCCGCTGATCAGCCTCG
ACTGTGCCTTCTA
SEQ ID NO: 202, MCP-linker-BAD-ADAR2-DD
(E488Q)-Bcl-xL-TagBFP Also known as
“BAD-DD-BclxL” and “WT” (see e.g., FIG. 21C)
MASNFTQFVLVDNGGTGDVTVAPSNFANGIAEWISSNSRSQAYKV
TCSVRQSSAQNRKYTIKVEVPKGAWRSYLNMELTIPIFATNSDCE
LIVKAMQGLLKDGNPIPSAIAANSGIYGGSGSGAGSGSPAGGGAP
GSGGGSTGAPPNLWAAQRYGRELRRMSDEFVDSFKKASQLHLPQV
LADAVSRLVLGKFGDLTDNFSSPHARRKVLAGVVMTTGTDVKDAK
VISVSTGTKCINGEYMSDRGLALNDCHAEIISRRSLLRFLYTQLE
LYLNNKDDQKRSIFQKSERGGFRLKENVQFHLYISTSPCGDARIF
SPHEPILEEPADRHPNRKARGQLRTKIESGQGTIPVRSNASIQTW
DGVLQGERLLTMSCSDKIARWNVVGIQGSLLSIFVEPIYFSSIIL
GSLYHGDHLSRAMYQRISNIEDLPPLYTLNKPLLSGISNAEARQP
GKAPNFSVNWTVGDSAIEVINATTGKDELGRASRLCKHALYCRWM
RVHGKVPSHLLRSKITKPNVYHESKLAAKEYQAAKARLFTAFIKA
GLGAWVEKPTEQDQFSLTGSAAASSNRELVVDFLSYKLSQKGYSW
SQFSDVEENRTEAPEGTESEMETPSAINGNPSWHLADSPAVNGAT
GHSSSLDAREVIPMAAVKQALREAGDEFELRYRRAFSDLTSQLHI
TPGTAYQSFEQVVNELFRDGVNWGRIVAFFSFGGALCVESVDKEM
QVLVSRIAAWMATYLNDHLEPWIQENGGWDTFVELYGNNGSSELI
KENMHMKLYMEGTVDNHHFKCTSEGEGKPYEGTQTMRIKVVEGGP
LPFAFDILATSFLYGSKTFINHTQGIPDFFKQSFPEGFTWERVTT
YEDGGVLTATQDTSLQDGCLIYNVKIRGVNFTSNGPVMQKKTLGW
EAFTETLYPADGGLEGRNDMALKLVGGSHLIANIKTTYRSKKPAK
NLKMPGVYYVDYRLERIKEANNETYVEQHEVAVARYCDLPSKLGH
KLN*
SEQ ID NO: 203,
TAATACGACTCACTATAGGGAGACCCAAGCTGGctagaggatcga
acccttaaggccaccatggcgtccaatttcactcagtttgtgctg
gttgacaacggcgggaccggggacgttacggtagccccctcaaac
tttgccaacggtatagcggagtggataagcagcaattctaggagt
caagcatacaaagttacatgcagcgtgcgccaatctagcgctcag
aatcgcaagtacaccattaaagtagaggtccccaagggagcctgg
agaagctatcttaacatggagttgaccataccaatcttcgctacc
aactctgactgtgaactcattgtgaaagccatgcaaggtctgctc
aaggatggtaacccaattccgtccgctatcgctgccaactctggg
atttacgggggcagtgggagcggtgcaggatctggtagtccagct
gggggaggagcaccgggtagcggtggggggtctACCGGTGCTCCA
CCCAATCTCTGGGCAGCGCAGCGCTACGGCCGTGAGCTCAGAAGG
ATGTCCGATGAGTTCGTCGATTCCTTCAAAAAGGCTAGCcagctg
cacctgccccaggttctcgcagacgccgtatcccgccttgtactg
ggcaagtttggtgatcttactgacaatttttcatctcctcatgcg
aggcggaaagtactcgcaggcgtcgtcatgacgaccggaactgac
gtgaaagacgccaaagtcatctctgtctccacgggcacaaagtgc
ataaacggggagtacatgagcgaccgggggctggcactgaatgat
tgtcacgctgaaataatatctaggcgatctctgcttagatttctc
tacactcaactcgaattgtaccttaacaacaaagatgaccagaaa
cgcagtatatttcagaaatcagaacgcggcggatttcgacttaag
gaaaacgttcagttccacttgtatatcagcacatccccttgcggt
gacgcccgaatcttttccccgcacgagccgatattggaggagccc
gcggacagacatcctaataggaaggctagaggccaacttcggacg
aagattgaaagtggccagggtactatcccggtgcggtccaacgct
agtattcaaacgtgggacggagtccttcaaggtgaacggctgttg
acaatgagctgctcagacaaaatcgcgcgctggaatgtagtggga
atccaaggcagcctcttgagcatattcgtagaacccatatatttc
tcatccattattttgggctctctgtatcatggtgaccatctgtca
agggctatgtaccaacgaatttctaatatcgaggatcttcctcca
ctctatacactcaataagcctctcttgtccgggatatcaaacgct
gaggcccgccagccagggaaagctcctaacttcagtgttaactgg
accgttggtgattctgcgatagaggtcatcaacgccacgacaggt
aaggatgagctcggtagagcctcacgcctgtgtaaacacgcgttg
tattgtagatggatgagagtacatgggaaggtcccatctcacttg
ctccgaagcaagatcactaagcctaatgtgtatcatgagtcaaaa
ctcgcggctaaagaataccaggcagccaaagctcgactttttaca
gcttttattaaggcagggctcggggcatgggtcgagaagccgacc
gagcaggaccaattctctctgacggggagcgcggccgccTCAAGT
AACCGGGAGCTGGTGGTTGACTTTCTCTCCTACAAGCTTTCCCAG
AAAGGATACAGCTGGAGTCAGTTTAGTGATGTGGAAGAGAACAGG
ACTGAGGCCCCAGAAGGGACTGAATCGGAGATGGAGACCCCCAGT
GCCATCAATGGCAACCCATCCTGGCACCTGGCAGACAGCCCCGCG
GTGAATGGAGCCACTGGCCACAGCAGCAGTTTGGATGCCCGGGAG
GTGATCCCCATGGCAGCAGTAAAGCAAGCGCTGAGGGAGGCAGGC
GACGAGTTTGAACTGCGGTACCGGCGGGCATTCAGTGACCTGACA
TCCCAGCTCCACATCACCCCAGGGACAGCATATCAGAGCTTTGAA
CAGGTAGTGAATGAACTCTTCCGGGATGGGGTAAACTGGGGTCGC
ATTGTGGCCTTTTTCTCCTTCGGCGGGGCACTGTGCGTGGAAAGC
GTAGACAAGGAGATGCAGGTATTGGTGAGTCGGATCGCAGCTTGG
ATGGCCACTTACCTGAATGACCACCTAGAGCCTTGGATCCAGGAG
AACGGCGGCTGGGATACTTTTGTGGAACTCTATGGGAACAATgga
tccAGCGAGCTGATTAAGGAGAACATGCACATGAAGCTGTACATG
GAGGGCACCGTGGACAACCATCACTTCAAGTGCACATCCGAGGGC
GAAGGCAAGCCCTACGAGGGCACCCAGACCATGAGAATCAAGGTG
GTCGAGGGCGGCCCTCTCCCCTTCGCCTTCGACATCCTGGCTACT
AGCTTCCTCTACGGCAGCAAGACCTTCATCAACCACACCCAGGGC
ATCCCCGACTTCTTCAAGCAGTCCTTCCCTGAGGGCTTCACATGG
GAGAGAGTCACCACATACGAAGACGGGGGCGTGCTGACCGCTACC
CAGGACACCAGCCTCCAGGACGGCTGCCTCATCTACAACGTCAAG
ATCAGAGGGGTGAACTTCACATCCAACGGCCCTGTGATGCAGAAG
AAAACACTCGGCTGGGAGGCCTTCACCGAGACGCTGTACCCCGCT
GACGGCGGCCTGGAAGGCAGAAACGACATGGCCCTGAAGCTCGTG
GGCGGGAGCCATCTGATCGCAAACATCAAGACCACATATAGATCC
AAGAAACCCGCTAAGAACCTCAAGATGCCTGGCGTCTACTATGTG
GACTACAGACTGGAAAGAATCAAGGAGGCCAACAACGAGACCTAC
GTCGAGCAGCACGAGGTGGCAGTGGCCAGATACTGCGACCTCCCT
AGCAAACTGGGGCACAAGCTTAATtAAGGGCCCGTTTAAACCCGC
TGATCAGCCTCGACTGTGCCTTCTA
SEQ ID NO: 204, tdMCP_ADAR2-DDN-CP5-46-4D5E_
ADAR2-DDC(E488Q)_mTagBFP
(AD-Pep-AD) (see e.g., FIG. 21D)
MASNFTQFVLVDNGGTGDVTVAPSNFANGIAEWISSNSRSQAYKV
TCSVRQSSAQNRKYTIKVEVPKGAWRSYLNMELTIPIFATNSDCE
LIVKAMQGLLKDGNPIPSAIAANSGIYANFTQFVLVDNGGTGDVT
VAPSNFANGIAEWISSNSRSQAYKVTCSVRQSSAQNRKYTIKVEV
PKGAWRSYLNMELTIPIFATNSDCELIVKAMQGLLKDGNPIPSAI
AANSGIYGGSGSGAGSGSPAGGGAPGSGGGSQLHLPQVLADAVSR
LVLGKFGDLTDNFSSPHARRKVLAGVVMTTGTDVKDAKVISVSTG
TKCINGEYMSDRGLALNDCHAEIISRRSLLRFLYTQLELYLNNKD
DQKRSIFQKSERGGFRLKENVQFHLYISTSPCGDARIFSPHEPIL
EEPASSGGELDELVYLLDGPGYDPIHCDVVTRGGSHLFNFDRHPN
RKARGQLRTKIESGQGTIPVRSNASIQTWDGVLQGERLLTMSCSD
KIARWNVVGIQGSLLSIFVEPIYFSSIILGSLYHGDHLSRAMYQR
ISNIEDLPPLYTLNKPLLSGISNAEARQPGKAPNFSVNWTVGDSA
IEVINATTGKDELGRASRLCKHALYCRWMRVHGKVPSHLLRSKIT
KPNVYHESKLAAKEYQAAKARLFTAFIKAGLGAWVEKPTEQDQFS
LTGSGSSELIKENMHMKLYMEGTVDNHHFKCTSEGEGKPYEGTQT
MRIKVVEGGPLPFAFDILATSFLYGSKTFINHTQGIPDFFKQSFP
EGFTWERVTTYEDGGVLTATQDTSLQDGCLIYNVKIRGVNFTSNG
PVMQKKTLGWEAFTETLYPADGGLEGRNDMALKLVGGSHLIANIK
TTYRSKKPAKNLKMPGVYYVDYRLERIKEANNETYVEQHEVAVAR
YCDLPSKLGHKLN*
SEQ ID NO: 205, Nucleic Acid-
TAATACGACTCACTATAGGGAGACCCAAGCTGGctagaggatcga
acccttaaggccaccatgGCGAGCAATTTTACCCAGTTTGTGCTT
GTGGACAACGGCGGCACCGGGGACGTGACGGTGGCCCCCTCCAAT
TTTGCCAATGGCATTGCAGAATGGATAAGCTCTAACAGCAGGAGC
CAGGCATACAAGGTGACCTGCAGCGTGAGGCAGTCAAGCGCTCAA
AACAGGAAGTACACCATTAAGGTCGAAGTGCCCAAAGGAGCTTGG
AGGTCTTACCTGAACATGGAACTGACAATTCCTATCTTCGCGACC
AATAGCGACTGTGAGCTGATCGTGAAGGCCATGCAAGGCCTGCTG
AAAGACGGGAATCCCATACCCAGCGCCATCGCCGCTAACTCAGGC
ATTTACGCTAATTTCACTCAGTTCGTACTGGTTGACAATGGGGGA
ACCGGCGACGTTACCGTGGCTCCAAGCAACTTCGCTAACGGGATC
GCCGAGTGGATCAGCAGTAATTCACGCTCCCAAGCCTACAAAGTA
ACCTGCTCTGTACGGCAGAGTTCAGCCCAGAACCGAAAGTATACC
ATCAAAGTGGAGGTGCCGAAGGGCGCCTGGCGGAGCTATCTGAAT
ATGGAGCTGACCATCCCCATCTTTGCCACGAACAGCGATTGCGAG
CTCATCGTCAAGGCGATGCAGGGCTTGCTGAAGGATGGCAACCCT
ATCCCGAGCGCAATAGCAGCCAACAGCGGCATCTATgggggcagt
gggagcggtgcaggatctggtagtccagctgggggaggagcaccg
ggtagcggtggggggtctcagctgcacctgccccaggttctcgca
gacgccgtatcccgccttgtactgggcaagtttggtgatcttact
gacaatttttcatctcctcatgcgaggcggaaagtactcgcaggc
gtcgtcatgacgaccggaactgacgtgaaagacgccaaagtcatc
tctgtctccacgggcacaaagtgcataaacggggagtacatgagc
gaccgggggctggcactgaatgattgtcacgctgaaataatatct
aggcgatctctgcttagatttctctacactcaactcgaattgtac
cttaacaacaaagatgaccagaaacgcagtatatttcagaaatca
gaacgcggcggatttcgacttaaggaaaacgttcagttccacttg
tatatcagcacatccccttgcggtgacgcccgaatcttttccccg
cacgagccgatattggaggagcccgcgTCGTCCGgtggagaactt
gatgaattggtatacttactagatgggccaggttatgaccctata
catTGCGATGTAGTGACAAGGGGCGGCAGCCACCTTTTCAATTTT
GACagacatcctaataggaaggctagaggccaacttcggacgaag
attgaaagtggccagggtactatcccggtgcggtccaacgctagt
attcaaacgtgggacggagtccttcaaggtgaacggctgttgaca
atgagctgctcagacaaaatcgcgcgctggaatgtagtgggaatc
caaggcagcctcttgagcatattcgtagaacccatatatttctca
tccattattttgggctctctgtatcatggtgaccatctgtcaagg
gctatgtaccaacgaatttctaatatcgaggatcttcctccactc
tatacactcaataagcctctcttgtccgggatatcaaacgctgag
gcccgccagccagggaaagctcctaacttcagtgttaactggacc
gttggtgattctgcgatagaggtcatcaacgccacgacaggtaag
gatgagctcggtagagcctcacgcctgtgtaaacacgcgttgtat
tgtagatggatgagagtacatgggaaggtcccatctcacttgctc
cgaagcaagatcactaagcctaatgtgtatcatgagtcaaaactc
gcggctaaagaataccaggcagccaaagctcgactttttacagct
tttattaaggcagggctcggggcatgggtcgagaagccgaccgag
caggaccaattctctctgacggggagcggatccAGCGAGCTGATT
AAGGAGAACATGCACATGAAGCTGTACATGGAGGGCACCGTGGAC
AACCATCACTTCAAGTGCACATCCGAGGGCGAAGGCAAGCCCTAC
GAGGGCACCCAGACCATGAGAATCAAGGTGGTCGAGGGCGGCCCT
CTCCCCTTCGCCTTCGACATCCTGGCTACTAGCTTCCTCTACGGC
AGCAAGACCTTCATCAACCACACCCAGGGCATCCCCGACTTCTTC
AAGCAGTCCTTCCCTGAGGGCTTCACATGGGAGAGAGTCACCACA
TACGAAGACGGGGGCGTGCTGACCGCTACCCAGGACACCAGCCTC
CAGGACGGCTGCCTCATCTACAACGTCAAGATCAGAGGGGTGAAC
TTCACATCCAACGGCCCTGTGATGCAGAAGAAAACACTCGGCTGG
GAGGCCTTCACCGAGACGCTGTACCCCGCTGACGGCGGCCTGGAA
GGCAGAAACGACATGGCCCTGAAGCTCGTGGGCGGGAGCCATCTG
ATCGCAAACATCAAGACCACATATAGATCCAAGAAACCCGCTAAG
AACCTCAAGATGCCTGGCGTCTACTATGTGGACTACAGACTGGAA
AGAATCAAGGAGGCCAACAACGAGACCTACGTCGAGCAGCACGAG
GTGGCAGTGGCCAGATACTGCGACCTCCCTAGCAAACTGGGGCAC
AAGCTTAATtAAGGGCCCGTTTAAACCCGCTGATCAGCCTCGACT
GTGCCTTCTA
SEQ ID NO: 206, tdMCP_ADAR2-
DDN-CP5-46-4D5E ADAR2-DDC(E488Q)_NS4A/NS3
(Genotype 1B)_mTagBFP (see e.g., FIG. 21E)
MASNFTQFVLVDNGGTGDVTVAPSNFANGIAEWISSNSRSQAYKV
TCSVRQSSAQNRKYTIKVEVPKGAWRSYLNMELTIPIFATNSDCE
LIVKAMQGLLKDGNPIPSAIAANSGIYANFTQFVLVDNGGTGDVT
VAPSNFANGIAEWISSNSRSQAYKVTCSVRQSSAQNRKYTIKVEV
PKGAWRSYLNMELTIPIFATNSDCELIVKAMQGLLKDGNPIPSAI
AANSGIYGGSGSGAGSGSPAGGGAPGSGGGSQLHLPQVLADAVSR
LVLGKFGDLTDNFSSPHARRKVLAGVVMTTGTDVKDAKVISVSTG
TKCINGEYMSDRGLALNDCHAEIISRRSLLRFLYTQLELYLNNKD
DQKRSIFQKSERGGFRLKENVQFHLYISTSPCGDARIFSPHEPIL
EEPASSGGELDELVYLLDGPGYDPIHCDVVTRGGSHLFNFDRHPN
RKARGQLRTKIESGQGTIPVRSNASIQTWDGVLQGERLLTMSCSD
KIARWNVVGIQGSLLSIFVEPIYFSSIILGSLYHGDHLSRAMYQR
ISNIEDLPPLYTLNKPLLSGISNAEARQPGKAPNFSVNWTVGDSA
IEVINATTGKDELGRASRLCKHALYCRWMRVHGKVPSHLLRSKIT
KPNVYHESKLAAKEYQAAKARLFTAFIKAGLGAWVEKPTEQDQFS
LTGSAAGGSGGSAAAQGSVVIVGRIILSGSGSITAYSQQTRGLLG
CIITSLTGRDKNQVEGEVQVVSTATQSFLATCVNGVCWTVYHGAG
SKTLAGPKGPITQMYTNVDQDLVGWQAPPGARSLTPCTCGSSDLY
LVTRHADVIPVRRRGDSRGSLLSPRPVSYLKGSSGGPLLCPSGHA
VGIFRAAVCTRGVAKAVDFVPVESMETTMRSESGSGTMSELIKEN
MHMKLYMEGTVDNHHFKCTSEGEGKPYEGTQTMRIKVVEGGPLPF
AFDILATSFLYGSKTFINHTQGIPDFFKQSFPEGFTWERVTTYED
GGVLTATQDTSLQDGCLIYNVKIRGVNFTSNGPVMQKKTLGWEAF
TETLYPADGGLEGRNDMALKLVGGSHLIANIKTTYRSKKPAKNLK
MPGVYYVDYRLERIKEANNETYVEQHEVAVARYCDLPSKLGHKLN
*
SEQ ID NO: 207,
TAATACGACTCACTATAGGGAGACCCAAGCTGGctagaggatcga
acccttaaggccaccatgGCGAGCAATTTTACCCAGTTTGTGCTT
GTGGACAACGGCGGCACCGGGGACGTGACGGTGGCCCCCTCCAAT
TTTGCCAATGGCATTGCAGAATGGATAAGCTCTAACAGCAGGAGC
CAGGCATACAAGGTGACCTGCAGCGTGAGGCAGTCAAGCGCTCAA
AACAGGAAGTACACCATTAAGGTCGAAGTGCCCAAAGGAGCTTGG
AGGTCTTACCTGAACATGGAACTGACAATTCCTATCTTCGCGACC
AATAGCGACTGTGAGCTGATCGTGAAGGCCATGCAAGGCCTGCTG
AAAGACGGGAATCCCATACCCAGCGCCATCGCCGCTAACTCAGGC
ATTTACGCTAATTTCACTCAGTTCGTACTGGTTGACAATGGGGGA
ACCGGCGACGTTACCGTGGCTCCAAGCAACTTCGCTAACGGGATC
GCCGAGTGGATCAGCAGTAATTCACGCTCCCAAGCCTACAAAGTA
ACCTGCTCTGTACGGCAGAGTTCAGCCCAGAACCGAAAGTATACC
ATCAAAGTGGAGGTGCCGAAGGGCGCCTGGCGGAGCTATCTGAAT
ATGGAGCTGACCATCCCCATCTTTGCCACGAACAGCGATTGCGAG
CTCATCGTCAAGGCGATGCAGGGCTTGCTGAAGGATGGCAACCCT
ATCCCGAGCGCAATAGCAGCCAACAGCGGCATCTATgggggcagt
gggagcggtgcaggatctggtagtccagctgggggaggagcaccg
ggtagcggtggggggtctcagctgcacctgccccaggttctcgca
gacgccgtatcccgccttgtactgggcaagtttggtgatcttact
gacaatttttcatctcctcatgcgaggcggaaagtactcgcaggc
gtcgtcatgacgaccggaactgacgtgaaagacgccaaagtcatc
tctgtctccacgggcacaaagtgcataaacggggagtacatgagc
gaccgggggctggcactgaatgattgtcacgctgaaataatatct
aggcgatctctgcttagatttctctacactcaactcgaattgtac
cttaacaacaaagatgaccagaaacgcagtatatttcagaaatca
gaacgcggcggatttcgacttaaggaaaacgttcagttccacttg
tatatcagcacatccccttgcggtgacgcccgaatcttttccccg
cacgagccgatattggaggagcccgcgTCGTCCGgtggagaactt
gatgaattggtatacttactagatgggccaggttatgaccctata
catTGCGATGTAGTGACAAGGGGCGGCAGCCACCTTTTCAATTTT
GACagacatcctaataggaaggctagaggccaacttcggacgaag
attgaaagtggccagggtactatcccggtgcggtccaacgctagt
attcaaacgtgggacggagtccttcaaggtgaacggctgttgaca
atgagctgctcagacaaaatcgcgcgctggaatgtagtgggaatc
caaggcagcctcttgagcatattcgtagaacccatatatttctca
tccattattttgggctctctgtatcatggtgaccatctgtcaagg
gctatgtaccaacgaatttctaatatcgaggatcttcctccactc
tatacactcaataagcctctcttgtccgggatatcaaacgctgag
gcccgccagccagggaaagctcctaacttcagtgttaactggacc
gttggtgattctgcgatagaggtcatcaacgccacgacaggtaag
gatgagctcggtagagcctcacgcctgtgtaaacacgcgttgtat
tgtagatggatgagagtacatgggaaggtcccatctcacttgctc
cgaagcaagatcactaagcctaatgtgtatcatgagtcaaaactc
gcggctaaagaataccaggcagccaaagctcgactttttacagct
tttattaaggcagggctcggggcatgggtcgagaagccgaccgag
caggaccaattctctctgacggggagcGCGGCCGGAGGTAGCGGC
GGAAGCGCGGCCGCTcaggggtctgttgttattgttggtagaatt
attttatctggtagtggtagtatcacggcctactcccaacagacg
cggggcctacttggttgcatcatcactagcctcacaggccgggac
aagaaccaggtcgaaggggaggttcaagtggtttctaccgcaaca
caatctttcctggcgacctgcgtcaacggcgtgtgctggactgtc
taccatggcgctggctcgaagaccctagccggtccaaaaggtcca
atcacccaaatgtacaccaatgtagaccaggacctcgtcggctgg
caggcgcctccaggggcgcgctccttgacaccatgcacctgtggc
agctcggacctttacttggtcacgagacatgctgatgtcattccg
gtgcgccggcgaggcgacagcaggggaagtctactctcccccagg
cccgtctcctacctgaaaggctccTCAggtggtccattgctttgc
ccttcggggcacgctgtgggcatcttccgggctgctgtgtgcacc
cggggggtcgcgaaggcggtggacttcgtgcccgttgagtctatg
gaaactaccatgcggtctGAGAGTGGATCAGGTACCATGAGCGAG
CTGATTAAGGAGAACATGCACATGAAGCTGTACATGGAGGGCACC
GTGAACAACCATCACTTCAAGTGCACATCCGAGGGCGAAGGCAAG
CCCTACGAGGGCACCCAGACCATGAGAATCAAGGTGGTCGAGGGC
GGCCCTCTCCCCTTCGCCTTCGACATCCTGGCTACTAGCTTCCTC
TACGGCAGCAAGACCTTCATCAACCACACCCAGGGCATCCCCGAC
TTCTTCAAGCAGTCCTTCCCTGAGGGCTTCACATGGGAGAGAGTC
ACCACATACGAAGACGGGGGCGTGCTGACCGCTACCCAGGACACC
AGCCTCCAGGACGGCTGCCTCATCTACAACGTCAAGATCAGAGGG
GTGAACTTCACATCCAACGGCCCTGTGATGCAGAAGAAAACACTC
GGCTGGGAGGCCTTCACCGAGACGCTGTACCCCGCTGACGGCGGC
CTGGAAGGCAGAAACGACATGGCCCTGAAGCTCGTGGGCGGGAGC
CATCTGATCGCAAACATCAAGACCACATATAGATCCAAGAAACCC
GCTAAGAACCTCAAGATGCCTGGCGTCTACTATGTGGACTACAGA
CTGGAAAGAATCAAGGAGGCCAACAACGAGACCTACGTCGAGCAG
CACGAGGTGGCAGTGGCCAGATACTGCGACCTCCCTAGCAAACTG
GGGCACAAGCTTAATTtAAGGGCCCGTTTAAACCCGCTGATCAGC
CTCGACTGTGCCTTCTA

### Example 18: Transfection of In Vitro Transcribed mRNA

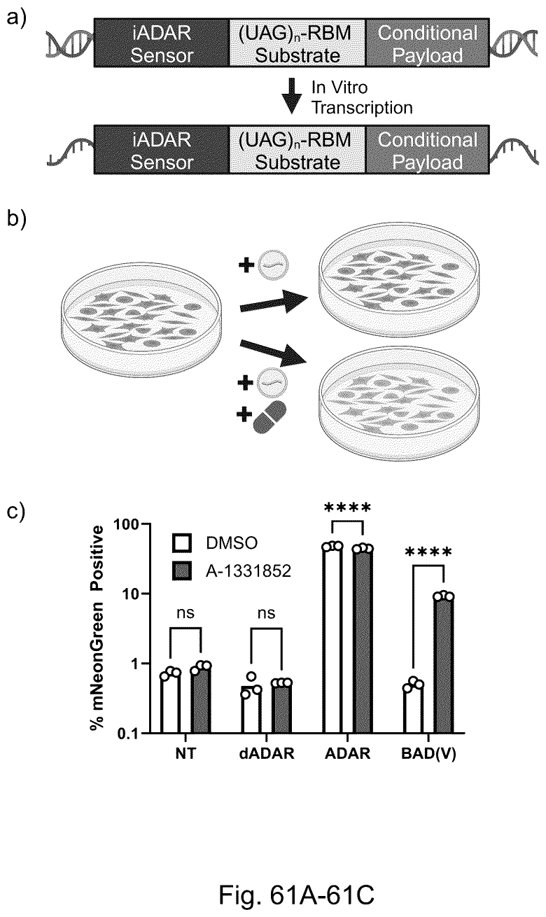

This example shows that constructs encoding an iADAR sensor and substrate on the same mRNA (see e.g., FIG. 61A) can be transcribed in vitro and delivered to cells as a single mRNA construct. To test this, iADAR constructs were cloned that contained the catalytically inactive ADAR2-DD (dADAR), constitutively active ADAR2-DD (ADAR), or conditionally active ADAR2-DD that is dependent on the addition of Bcl inhibitor A-1331852 (BAD(V)) upstream of editable stop codons and an mNeonGreen fluorescent protein (see e.g., FIG. 61A). In this case, the dADAR and ADAR constructs serve as negative and positive controls respectively. For the BAD(V) construct, cells only express mNeonGreen if A-1331852 is present during the transfection (see e.g., FIG. 61B).

To test this, the 3′ end of the plasmid was subsequently digested using PmeI, a DNA clean-up was performed, ARCA-capped mRNA was transcribed with T7 polymerase, the remaining DNA were digested with DNAse I, and a poly-A tail was added with E. coli Poly(A) Polymerase (all reagents and protocols from NEW ENGLAND BIO's HiScribe® T7 ARCA mRNA Kit (with tailing)). 70,000 HEK293FT cells were then transfected in suspension with 100 ng of each mRNA construct using Lipofectamine™ MessengerMAX™ Transfection Reagent. Cells were also treated with either DMSO or 1 μM of A-1331852. ˜48 hours after transfection, cells were analyzed by flow cytometry. mNeonGreen positive cells were determined as greater than the top 1% of gated non-transfected HEK cells (NT).

There was an insignificant population of mNeonGreen positive cells for the non-transfected cells (NT) or dADAR negative control, and a significant population of mNeonGreen positive cells for the active ADAR case (see e.g., FIG. 61C). The extent of mNeonGreen positive cells for the BAD(V) construct was dependent on the presence of A-1331852 (see e.g., FIG. 61C), with an increase of ˜18× when A-1331852 was present.

Combined, these data demonstrate that the iADAR system is compatible with a delivery system where in-vitro transcribed mRNA is delivered directly to cells.

### DNA Sequences

SEQ ID NO: 390, MCP_ADAR2-DD(E396A &
E488Q)_mCherry_FLAG_P2A_T2A_loop(UAG-UAG)_
MS2(C)_HaloTag_loop(UAG-UAG)_MS2(C)_
P2A_T2A HA_mNeonGreen(M10K)
TAATACGACTCACTATAGGGAGACCCAAGCTGGctagaggatcga
acccttaaggccaccatggcgtccaatttcactcagtttgtgctg
gttgacaacggcgggaccggggacgttacggtagccccctcaaac
tttgccaacggtatagcggagtggataagcagcaattctaggagt
caagcatacaaagttacatgcagcgtgcgccaatctagcgctcag
aatcgcaagtacaccattaaagtagaggtccccaagggagcctgg
agaagctatcttaacatggagttgaccataccaatcttcgctacc
aactctgactgtgaactcattgtgaaagccatgcaaggtctgctc
aaggatggtaacccaattccgtccgctatcgctgccaactctggg
atttacgggggcagtgggagcggtgcaggatctggtagtccagct
gggggaggagcaccgggtagcggtggggggtctcagctgcacctg
ccccaggttctcgcagacgccgtatcccgccttgtactgggcaag
tttggtgatcttactgacaatttttcatctcctcatgcgaggcgg
aaagtactcgcaggcgtcgtcatgacgaccggaactgacgtgaaa
gacgccaaagtcatctctgtctccacgggcacaaagtgcataaac
ggggagtacatgagcgaccgggggctggcactgaatgattgtcac
gctGCCataatatctaggcgatctctgcttagatttctctacact
caactcgaattgtaccttaacaacaaagatgaccagaaacgcagt
atatttcagaaatcagaacgcggcggatttcgacttaaggaaaac
gttcagttccacttgtatatcagcacatccccttgcggtgacgcc
cgaatcttttccccgcacgagccgatattggaggagcccgcggac
agacatcctaataggaaggctagaggccaacttcggacgaagatt
gaaagtggccagggtactatcccggtgcggtccaacgctagtatt
caaacgtgggacggagtccttcaaggtgaacggctgttgacaatg
agctgctcagacaaaatcgcgcgctggaatgtagtgggaatccaa
ggcagcctcttgagcatattcgtagaacccatatatttctcatcc
attattttgggctctctgtatcatggtgaccatctgtcaagggct
atgtaccaacgaatttctaatatcgaggatcttcctccactctat
acactcaataagcctctcttgtccgggatatcaaacgctgaggcc
cgccagccagggaaagctcctaacttcagtgttaactggaccgtt
ggtgattctgcgatagaggtcatcaacgccacgacaggtaaggat
gagctcggtagagcctcacgcctgtgtaaacacgcgttgtattgt
agatggatgagagtacatgggaaggtcccatctcacttgctccga
agcaagatcactaagcctaatgtgtatcatgagtcaaaactcgcg
gctaaagaataccaggcagccaaagctcgactttttacagctttt
attaaggcagggctcggggcatgggtcgagaagccgaccgagcag
gaccaattctctctgacggggagcggatccAGCGAGCTGATTAAG
GAGAACATGCACATGAAGCGCCCatcggtcgccaccatggtgagc
aagggcgaggaggataacatggccatcatcaaggagttcatgcgc
ttcaaggtgcacatggagggctccgtgaacggccacgagttcgag
atcgagggcgagggcgagggccgcccctacgagggcacccagacc
gccaagctgaaggtgaccaagggtggccccctgcccttcgcctgg
gacatcctgtcccctcagttcatgtacggctccaaggcctacgtg
aagcaccccgccgacatccccgactacttgaagctgtccttcccc
gagggcttcaagtgggagcgcgtgatgaacttcgaggacggcggc
gtggtgaccgtgacccaggactcctccctgcaggacggcgagttc
atctacaaggtgaagctgcgcggcaccaacttcccctccgacggc
cccgtaatgcagaagaagaccatgggctgggaggcctcctccgag
cggatgtaccccgaggacggcgccctgaagggcgagatcaagcag
aggctgaagctgaaggacggcggccactacgacgctgaggtcaag
accacctacaaggccaagaagcccgtgcagctgcccggcgcctac
aacgtcaacatcaagttggacatcacctcccacaacgaggactac
accatcgtggaacagtacgaacgcgccgagggccgccactccacc
ggcggcatggacgagctgTACaaggattacaaggatgacgatga
caaaGGTAGCGGGGCAACTAATTTTAGCTTACTCAAACAGGCTGG
GGACGTCGAGGAGAATCCAGGCCCTGCATCCGCTGGCTCTGGAGA
AGGACGAGGCTCCTTGCTCACCTGTGGAGATGTCGAAGAGAACCC
AGGTCCTGCAACCGGGAATTCCGCGTAGCGCTAGCTTTGCCAGCG
CCACGCGaaACATGAGGATcACCCATGTGCCGCTATGGCAGAAAT
CGGTACTGGCTTTCCATTCGACCCCCATTATGTGGAAGTCCTGGG
CGAGCGCATGCACTACGTCGATGTTGGTCCGCGCGATGGCACCCC
TGTGCTGTTCCTGCACGGTAACCCGACCTCCTCCTACGTGTGGCG
CAACATCATCCCGCATGTTGCACCGACCCATCGCTGCATTGCTCC
AGACCTGATCGGTATGGGCAAATCCGACAAACCAGACCTGGGTTA
TTTCTTCGACGACCACGTCCGCTTCATGGATGCCTTCATCGAAGC
CCTGGGTCTGGAAGAGGTCGTCCTGGTCATTCACGACTGGGGCTC
CGCTCTGGGTTTCCACTGGGCCAAGCGCAATCCAGAGCGCGTCAA
AGGTATTGCATTTATGGAGTTCATCCGCCCTATCCCGACCTGGGA
CGAATGGCCAGAATTTGCCCGCGAGACCTTCCAGGCCTTCCGCAC
CACCGACGTCGGCCGCAAGCTGATCATCGATCAGAACGTTTTTAT
CGAGGGTACGCTGCCGATGGGTGTCGTCCGCCCGCTGACTGAAGT
CGAGATGGACCATTACCGCGAGCCGTTCCTGAATCCTGTTGACCG
CGAGCCACTGTGGCGCTTCCCAAACGAGCTGCCAATCGCCGGTGA
GCCAGCGAACATCGTCGCGCTGGTCGAAGAATACATGGACTGGCT
GCACCAGTCCCCTGTCCCGAAGCTGCTGTTCTGGGGCACCCCAGG
CGTTCTGATCCCACCGGCCGAAGCCGCTCGCCTGGCCAAAAGCCT
GCCTAACTGCAAGGCTGTGGACATCGGCCCGGGTCTGAATCTGCT
GCAAGAAGACAACCCGGACCTGATCGGCAGCGAGATCGCGCGCTG
GCTGTCGACGCTCGAGATTTCTGGCACCGGTATGGCATCTATGAC
TGGAGGCCAACAGATGgGTCCTGCAACCGGGAATTCCGCGTAGCG
CTAGCTTTGCCAGCGCCACGCGaaACATGAGGATcACCCATGTAC
TAGTGCCACAAACTTCTCTCTGCTAAAGCAAGCAGGTGATGTTGA
AGAAAACCCAGGGCCTGGAGGGTCCGAGGGCAGGGGAAGTCTCCT
AACATGCGGGGACGTGGAGGAAAATCCCGGCCCATCCGGATATCC
CTACGATGTGCCCGATTACGCTATCGATgtgagcaagggcgaAga
AgataacaAggcctctctcccagcgacacatgagttacacatctt
tggctccatcaacggtgtggactttgacatggtgggtcagggcac
cggcaatccaaatgatggttatgaggagttaaacctgaagtccac
caagggtgacctccagttctccccctggattctggtccctcatat
cgggtatggcttccatcagtacctgccctaccctgacgggatgtc
gcctttccaggccgccatggtagatggcAGCggataccaagtcca
tcgcacaatgcagtttgaagatggtgcctcccttactgttaacta
ccgctacacctacgagggaagccacatcaaaggagaggcccaggt
gaaggggactggtttccctgctgacggtcctgtgatgaccaactc
gctgaccgctgcggactggtgcaggtcgaagaagacttaccccaa
cgacaaaaccatcatcagtacctttaagtggagttacaccactgg
aaatggcaagAGAtaccggagcactgcgcggaccacctacacctt
tgccaagccaatggcggctaactatctgaagaaccagccgatgta
cgtgttccgtaagacggagctcaagcactccaagaccgagctcaa
cttcaaggagtggcaaaaggcctttaccgatgtgatgGGAatgga
cGAGCTGTATaagGCTAGCTAAGCGGCCGCTCGAGTCTAGAGGGC
CCGCGGTTCGAAGGTAAGCCTATCCCTAACCCTCTCCTCGGTCTC
GATTCTACGCGTACCGGTCATCATCACCATCACCATTGAGTTTAA
ACCCGCTGATCAGCCTCGACTGTGCCTTCTA
SEQ ID NO: 391, MCP_ADAR2-DD
(E488Q)_mCherry_FLAG_P2A_T2A_loop(UAG-
UAG)_MS2(C)_HaloTag_loop(UAG-UAG)_MS2(C)_
P2A_T2A_HA_mNeonGreen(M10K)
TAATACGACTCACTATAGGGAGACCCAAGCTGGctagaggatcga
acccttaaggccaccatggcgtccaatttcactcagtttgtgctg
gttgacaacggcgggaccggggacgttacggtagccccctcaaac
tttgccaacggtatagcggagtggataagcagcaattctaggagt
caagcatacaaagttacatgcagcgtgcgccaatctagcgctcag
aatcgcaagtacaccattaaagtagaggtccccaagggagcctgg
agaagctatcttaacatggagttgaccataccaatcttcgctacc
aactctgactgtgaactcattgtgaaagccatgcaaggtctgctc
aaggatggtaacccaattccgtccgctatcgctgccaactctggg
atttacgggggcagtgggagcggtgcaggatctggtagtccagct
gggggaggagcaccgggtagcggtggggggtctcagctgcacctg
ccccaggttctcgcagacgccgtatcccgccttgtactgggcaag
tttggtgatcttactgacaatttttcatctcctcatgcgaggcgg
aaagtactcgcaggcgtcgtcatgacgaccggaactgacgtgaaa
gacgccaaagtcatctctgtctccacgggcacaaagtgcataaac
ggggagtacatgagcgaccgggggctggcactgaatgattgtcac
gctgaaataatatctaggcgatctctgcttagatttctctacact
caactcgaattgtaccttaacaacaaagatgaccagaaacgcagt
atatttcagaaatcagaacgcggcggatttcgacttaaggaaaac
gttcagttccacttgtatatcagcacatccccttgcggtgacgcc
cgaatcttttccccgcacgagccgatattggaggagcccgcggac
agacatcctaataggaaggctagaggccaacttcggacgaagatt
gaaagtggccagggtactatcccggtgcggtccaacgctagtatt
caaacgtgggacggagtccttcaaggtgaacggctgttgacaatg
agctgctcagacaaaatcgcgcgctggaatgtagtgggaatccaa
ggcagcctcttgagcatattcgtagaacccatatatttctcatcc
attattttgggctctctgtatcatggtgaccatctgtcaagggct
atgtaccaacgaatttctaatatcgaggatcttcctccactctat
acactcaataagcctctcttgtccgggatatcaaacgctgaggcc
cgccagccagggaaagctcctaacttcagtgttaactggaccgtt
ggtgattctgcgatagaggtcatcaacgccacgacaggtaaggat
gagctcggtagagcctcacgcctgtgtaaacacgcgttgtattgt
agatggatgagagtacatgggaaggtcccatctcacttgctccga
agcaagatcactaagcctaatgtgtatcatgagtcaaaactcgcg
gctaaagaataccaggcagccaaagctcgactttttacagctttt
attaaggcagggctcggggcatgggtcgagaagccgaccgagcag
gaccaattctctctgacggggagcggatccAGCGAGCTGATTAAG
GAGAACATGCACATGAAGCGCCCatcggtcgccaccatggtgagc
aagggcgaggaggataacatggccatcatcaaggagttcatgcgc
ttcaaggtgcacatggagggctccgtgaacggccacgagttcgag
atcgagggcgagggcgagggccgcccctacgagggcacccagacc
gccaagctgaaggtgaccaagggtggccccctgcccttcgcctgg
gacatcctgtcccctcagttcatgtacggctccaaggcctacgtg
aagcaccccgccgacatccccgactacttgaagctgtccttcccc
gagggcttcaagtgggagcgcgtgatgaacttcgaggacggcggc
gtggtgaccgtgacccaggactcctccctgcaggacggcgagttc
atctacaaggtgaagctgcgcggcaccaacttcccctccgacggc
cccgtaatgcagaagaagaccatgggctgggaggcctcctccgag
cggatgtaccccgaggacggcgccctgaagggcgagatcaagcag
aggctgaagctgaaggacggcggccactacgacgctgaggtcaag
accacctacaaggccaagaagcccgtgcagctgcccggcgcctac
aacgtcaacatcaagttggacatcacctcccacaacgaggactac
accatcgtggaacagtacgaacgcgccgagggccgccactccacc
ggcggcatggacgagctgTACaaggattacaaggatgacgatga
caaaGGTAGCGGGGCAACTAATTTTAGCTTACTCAAACAGGCTGG
GGACGTCGAGGAGAATCCAGGCCCTGCATCCGCTGGCTCTGGAGA
AGGACGAGGCTCCTTGCTCACCTGTGGAGATGTCGAAGAGAACCC
AGGTCCTGCAACCGGGAATTCCGCGTAGCGCTAGCTTTGCCAGCG
CCACGCGaaACATGAGGATcACCCATGTGCCGCTATGGCAGAAAT
CGGTACTGGCTTTCCATTCGACCCCCATTATGTGGAAGTCCTGGG
CGAGCGCATGCACTACGTCGATGTTGGTCCGCGCGATGGCACCCC
TGTGCTGTTCCTGCACGGTAACCCGACCTCCTCCTACGTGTGGCG
CAACATCATCCCGCATGTTGCACCGACCCATCGCTGCATTGCTCC
AGACCTGATCGGTATGGGCAAATCCGACAAACCAGACCTGGGTTA
TTTCTTCGACGACCACGTCCGCTTCATGGATGCCTTCATCGAAGC
CCTGGGTCTGGAAGAGGTCGTCCTGGTCATTCACGACTGGGGCTC
CGCTCTGGGTTTCCACTGGGCCAAGCGCAATCCAGAGCGCGTCAA
AGGTATTGCATTTATGGAGTTCATCCGCCCTATCCCGACCTGGGA
CGAATGGCCAGAATTTGCCCGCGAGACCTTCCAGGCCTTCCGCAC
CACCGACGTCGGCCGCAAGCTGATCATCGATCAGAACGTTTTTAT
CGAGGGTACGCTGCCGATGGGTGTCGTCCGCCCGCTGACTGAAGT
CGAGATGGACCATTACCGCGAGCCGTTCCTGAATCCTGTTGACCG
CGAGCCACTGTGGCGCTTCCCAAACGAGCTGCCAATCGCCGGTGA
GCCAGCGAACATCGTCGCGCTGGTCGAAGAATACATGGACTGGCT
GCACCAGTCCCCTGTCCCGAAGCTGCTGTTCTGGGGCACCCCAGG
CGTTCTGATCCCACCGGCCGAAGCCGCTCGCCTGGCCAAAAGCCT
GCCTAACTGCAAGGCTGTGGACATCGGCCCGGGTCTGAATCTGCT
GCAAGAAGACAACCCGGACCTGATCGGCAGCGAGATCGCGCGCTG
GCTGTCGACGCTCGAGATTTCTGGCACCGGTATGGCATCTATGAC
TGGAGGCCAACAGATGgGTCCTGCAACCGGGAATTCCGCGTAGCG
CTAGCTTTGCCAGCGCCACGCGaaACATGAGGATcACCCATGTAC
TAGTGCCACAAACTTCTCTCTGCTAAAGCAAGCAGGTGATGTTGA
AGAAAACCCAGGGCCTGGAGGGTCCGAGGGCAGGGGAAGTCTCCT
AACATGCGGGGACGTGGAGGAAAATCCCGGCCCATCCGGATATCC
CTACGATGTGCCCGATTACGCTATCGATgtgagcaagggcgaAga
AgataacaAggcctctctcccagcgacacatgagttacacatctt
tggctccatcaacggtgtggactttgacatggtgggtcagggcac
cggcaatccaaatgatggttatgaggagttaaacctgaagtccac
caagggtgacctccagttctccccctggattctggtccctcatat
cgggtatggcttccatcagtacctgccctaccctgacgggatgtc
gcctttccaggccgccatggtagatggcAGCggataccaagtcca
tcgcacaatgcagtttgaagatggtgcctcccttactgttaacta
ccgctacacctacgagggaagccacatcaaaggagaggcccaggt
gaaggggactggtttccctgctgacggtcctgtgatgaccaactc
gctgaccgctgcggactggtgcaggtcgaagaagacttaccccaa
cgacaaaaccatcatcagtacctttaagtggagttacaccactgg
aaatggcaagAGAtaccggagcactgcgcggaccacctacacctt
tgccaagccaatggggctaactatctgaagaaccagccgatgtac
gtgttccgtaagacggagctcaagcactccaagaccgagctcaac
ttcaaggagtggcaaaaggcctttaccgatgtgatgGGAatggac
GAGCTGTATaagGCTAGCTAAGCGGCCGCTCGAGTCTAGAGGGCC
CGCGGTTCGAAGGTAAGCCTATCCCTAACCCTCTCCTCGGTCTCG
ATTCTACGCGTACCGGTCATCATCACCATCACCATTGAGTTTAAA
CCCGCTGATCAGCCTCGACTGTGCCTTCTA
SEQ ID NO: 392, MCP_ADAR2-DDN_BAD
(V)_ADAR2-DDC(E488Q & K672R)_Bcl-
xL_mCherry_FLAG_P2A_T2A_loop(UAG-UAG)_MS2(C)_
HaloTag_loop(UAG-UAG)_MS2(C)_P2A_T2A
HA_mNeonGreen(M10K)
TAATACGACTCACTATAGGGAGACCCAAGCTGGctagaggatcga
acccttaaggccaccatggcgtccaatttcactcagtttgtgctg
gttgacaacggcgggaccggggacgttacggtagccccctcaaac
tttgccaacggtatagcggagtggataagcagcaattctaggagt
caagcatacaaagttacatgcagcgtgcgccaatctagcgctcag
aatcgcaagtacaccattaaagtagaggtccccaagggagcctgg
agaagctatcttaacatggagttgaccataccaatcttcgctacc
aactctgactgtgaactcattgtgaaagccatgcaaggtctgctc
aaggatggtaacccaattccgtccgctatcgctgccaactctggg
atttacgggggcagtgggagcggtgcaggatctggtagtccagct
gggggaggagcaccgggtagcggtggggggtctcagctgcacctg
ccccaggttctcgcagacgccgtatcccgccttgtactgggcaag
tttggtgatcttactgacaatttttcatctcctcatgcgaggcgg
aaagtactcgcaggcgtcgtcatgacgaccggaactgacgtgaaa
gacgccaaagtcatctctgtctccacgggcacaaagtgcataaac
ggggagtacatgagcgaccgggggctggcactgaatgattgtcac
gctgaaataatatctaggcgatctctgcttagatttctctacact
caactcgaattgtaccttaacaacaaagatgaccagaaacgcagt
atatttcagaaatcagaacgcggcggatttcgacttaaggaaaac
gttcagttccacttgtatatcagcacatccccttgcggtgacgcc
cgaatcttttccccgcacgagccgatattggaggagcccgcgGCT
AGCGGGTCGGGCACCGGTGCTCCACCCAATCTCTGGGCAGCGCAG
CGCTACGGCCGTGAGCTCAGAAGGATGTCCGATGAGGTGGTCGAC
agacatcctaataggaaggctagaggccaacttcggacgaagatt
gaaagtggccagggtactatcccggtgcggtccaacgctagtatt
caaacgtgggacggagtccttcaaggtgaacggctgttgacaatg
agctgctcagacaaaatcgcgcgctggaatgtagtgggaatccaa
ggcagcctcttgagcatattcgtagaacccatatatttctcatcc
attattttgggctctctgtatcatggtgaccatctgtcaagggct
atgtaccaacgaatttctaatatcgaggatcttcctccactctat
acactcaataagcctctcttgtccgggatatcaaacgctgaggcc
cgccagccagggaaagctcctaacttcagtgttaactggaccgtt
ggtgattctgcgatagaggtcatcaacgccacgacaggtaaggat
gagctcggtagagcctcacgcctgtgtaaacacgcgttgtattgt
agatggatgagagtacatgggaaggtcccatctcacttgctccga
agcaagatcactaagcctaatgtgtatcatgagtcaaaactcgcg
gctaaagaataccaggcagccaGGgctcgactttttacagctttt
attaaggcagggctcggggcatgggtcgagaagccgaccgagcag
gaccaattctctctgacggggagcGCGGCCGGAGGTAGCGGCGGA
AGCGCGGCCGCCTCAAGTAACCGGGAGCTGGTGGTTGACTTTCTC
TCCTACAAGCTTTCCCAGAAAGGATACAGCTGGAGTCAGTTTAGT
GATGTGGAAGAGAACAGGACTGAGGCCCCAGAAGGGACTGAATCG
GAGATGGAGACCCCCAGTGCCATCAATGGCAACCCATCCTGGCAC
CTGGCAGACAGCCCCGCGGTGAATGGAGCCACTGGCCACAGCAGC
AGTTTGGATGCCCGGGAGGTGATCCCCATGGCAGCAGTAAAGCAA
GCGCTGAGGGAGGCAGGCGACGAGTTTGAACTGCGGTACCGGCGG
GCATTCAGTGACCTGACATCCCAGCTCCACATCACCCCAGGGACA
GCATATCAGAGCTTTGAACAGGTAGTGAATGAACTCTTCCGGGAT
GGGGTAAACTGGGGTCGCATTGTGGCCTTTTTCTCCTTCGGCGGG
GCACTGTGCGTGGAAAGCGTAGACAAGGAGATGCAGGTATTGGTG
AGTCGGATCGCAGCTTGGATGGCCACTTACCTGAATGACCACCTA
GAGCCTTGGATCCAGGAGAACGGCGGCTGGGATACTTTTGTGGAA
CTCTATGGGAACAATggatccAGCGAGCTGATTAAGGAGAACATG
CACATGAAGCGCCCatcggtcgccaccatggtgagcaagggcgag
gaggataacatggccatcatcaaggagttcatgcgcttcaaggtg
cacatggagggctccgtgaacggccacgagttcgagatcgagggc
gagggcgagggccgcccctacgagggcacccagaccgccaagctg
aaggtgaccaagggtggccccctgcccttcgcctgggacatcctg
tcccctcagttcatgtacggctccaaggcctacgtgaagcacccc
gccgacatccccgactacttgaagctgtccttccccgagggcttc
aagtgggagcgcgtgatgaacttcgaggacggcggcgtggtgacc
gtgacccaggactcctccctgcaggacggcgagttcatctacaag
gtgaagctgcgcggcaccaacttcccctccgacggccccgtaatg
cagaagaagaccatgggctgggaggcctcctccgagcggatgtac
cccgaggacggcgccctgaagggcgagatcaagcagaggctgaag
ctgaaggacggcggccactacgacgctgaggtcaagaccacctac
aaggccaagaagcccgtgcagctgcccggcgcctacaacgtcaac
atcaagttggacatcacctcccacaacgaggactacaccatcgtg
gaacagtacgaacgcgccgagggccgccactccaccggcggcatg
gacgagctgTACaaggattacaaggatgacgatgacaaaGGTAGC
GGGGCAACTAATTTTAGCTTACTCAAACAGGCTGGGGACGTCGAG
GAGAATCCAGGCCCTGCATCCGCTGGCTCTGGAGAAGGACGAGGC
TCCTTGCTCACCTGTGGAGATGTCGAAGAGAACCCAGGTCCTGCA
ACCGGGAATTCCGCGTAGCGCTAGCTTTGCCAGCGCCACGCGaaA
CATGAGGATcACCCATGTGCCGCTATGGCAGAAATCGGTACTGGC
TTTCCATTCGACCCCCATTATGTGGAAGTCCTGGGCGAGCGCATG
CACTACGTCGATGTTGGTCCGCGCGATGGCACCCCTGTGCTGTTC
CTGCACGGTAACCCGACCTCCTCCTACGTGTGGCGCAACATCATC
CCGCATGTTGCACCGACCCATCGCTGCATTGCTCCAGACCTGATC
GGTATGGGCAAATCCGACAAACCAGACCTGGGTTATTTCTTCGAC
GACCACGTCCGCTTCATGGATGCCTTCATCGAAGCCCTGGGTCTG
GAAGAGGTCGTCCTGGTCATTCACGACTGGGGCTCCGCTCTGGGT
TTCCACTGGGCCAAGCGCAATCCAGAGCGCGTCAAAGGTATTGCA
TTTATGGAGTTCATCCGCCCTATCCCGACCTGGGACGAATGGCCA
GAATTTGCCCGCGAGACCTTCCAGGCCTTCCGCACCACCGACGTC
GGCCGCAAGCTGATCATCGATCAGAACGTTTTTATCGAGGGTACG
CTGCCGATGGGTGTCGTCCGCCCGCTGACTGAAGTCGAGATGGAC
CATTACCGCGAGCCGTTCCTGAATCCTGTTGACCGCGAGCCACTG
TGGCGCTTCCCAAACGAGCTGCCAATCGCCGGTGAGCCAGCGAAC
ATCGTCGCGCTGGTCGAAGAATACATGGACTGGCTGCACCAGTCC
CCTGTCCCGAAGCTGCTGTTCTGGGGCACCCCAGGCGTTCTGATC
CCACCGGCCGAAGCCGCTCGCCTGGCCAAAAGCCTGCCTAACTGC
AAGGCTGTGGACATCGGCCCGGGTCTGAATCTGCTGCAAGAAGAC
AACCCGGACCTGATCGGCAGCGAGATCGCGCGCTGGCTGTCGACG
CTCGAGATTTCTGGCACCGGTATGGCATCTATGACTGGAGGCCAA
CAGATGgGTCCTGCAACCGGGAATTCCGCGTAGCGCTAGCTTTGC
CAGCGCCACGCGaaACATGAGGATcACCCATGTACTAGTGCCACA
AACTTCTCTCTGCTAAAGCAAGCAGGTGATGTTGAAGAAAACCCA
GGGCCTGGAGGGTCCGAGGGCAGGGGAAGTCTCCTAACATGCGGG
GACGTGGAGGAAAATCCCGGCCCATCCGGATATCCCTACGATGTG
CCCGATTACGCTATCGATgtgagcaagggcgaAgaAgataacaAg
gcctctctcccagcgacacatgagttacacatctttggctccatc
aacggtgtggactttgacatggtgggtcagggcaccggcaatcca
aatgatggttatgaggagttaaacctgaagtccaccaagggtgac
ctccagttctccccctggattctggtccctcatatcgggtatggc
ttccatcagtacctgccctaccctgacgggatgtcgcctttccag
gccgccatggtagatggcAGCggataccaagtccatcgcacaatg
cagtttgaagatggtgcctcccttactgttaactaccgctacacc
tacgagggaagccacatcaaaggagaggcccaggtgaaggggact
ggtttccctgctgacggtcctgtgatgaccaactcgctgaccgct
gcggactggtgcaggtcgaagaagacttaccccaacgacaaaacc
atcatcagtacctttaagtggagttacaccactggaaatggcaag
AGAtaccggagcactgcgcggaccacctacacctttgccaagcca
atggcggctaactatctgaagaaccagccgatgtacgtgttccgt
aagacggagctcaagcactccaagaccgagctcaacttcaaggag
tggcaaaaggcctttaccgatgtgatgGGAatggacGAGCTGTAT
aagGCTAGCTAAGCGGCCGCTCGAGTCTAGAGGGCCCGCGGTTCG
AAGGTAAGCCTATCCCTAACCCTCTCCTCGGTCTCGATTCTACGC
GTACCGGTCATCATCACCATCACCATTGAGTTTAAACCCGCTGAT
CAGCCTCGACTGTGCCTTCTA

### REFERENCES

The disclosures of all publications, patents, patent applications and published patent applications referred to herein are hereby incorporated herein by reference in their entirety.

US20210355494

WO2023020574

US20200263180

Kügler, J. et al. High Affinity Peptide Inhibitors of the Hepatitis C Virus NS3-4A Protease Refractory to Common Resistant Mutants. J Biol Chem 287, 39224-39232 (2012).

Tague, E. P., McMahan, J. B., Tague, N., Dunlop, M. J. & Ngo, J. T. Controlled Protein Activities with Viral Proteases, Antiviral Peptides, and Antiviral Drugs. Acs Chem Biol 18, 1228-1236 (2023).

D. Cunningham-Bryant, E. M. Dieter, G. W. Foight, J. C. Rose, D. E. Loutey, D. J. Maly, A Chemically Disrupted Proximity System for Controlling Dynamic Cellular Processes. J Am Chem Soc. 141, 3352-3355 (2019).

Jiang, K. et al. Programmable eukaryotic protein synthesis with RNA sensors by harnessing ADAR. Nat Biotechnol 1-10 (2022) doi:10.1038/s41587-022-01534-5.

Katrekar, D. et al. In vivo RNA editing of point mutations via RNA-guided adenosine deaminases. Nat Methods 16, 239-242 (2019).

Klauer, A. A. & Hoof, A. van. Degradation of mRNAs that lack a stop codon: a decade of nonstop progress. Wiley Interdiscip Rev RNA 3, 649-660 (2012)

Matthews, M. M. et al. Structures of human ADAR2 bound to dsRNA reveal base-flipping mechanism and basis for site selectivity. Nat Struct Mol Biol 23, 426-433 (2016).

Park, S. et al. High-throughput mutagenesis reveals unique structural features of human ADAR1. Nat Commun 11, 5130 (2020).

Katrekar, D. et al. Comprehensive interrogation of the ADAR2 deaminase domain for engineering enhanced RNA editing activity and specificity. Elife 11, e75555 (2022).

Goreshnik, I. & Maly, D. J. A Small Molecule-Regulated Guanine Nucleotide Exchange Factor. J Am Chem Soc 132, 938-940 (2010).

Rose, J. C. et al. Rapidly inducible Cas9 and DSB-ddPCR to probe editing kinetics. Nat Methods 14, 891-896 (2017).

Wang, L. et al. Discovery of A 1331852, a First-in-Class, Potent, and Orally-Bioavailable BCL XL Inhibitor. Acs Med Chem Lett 11, 1829-1836 (2020).

Kotschy, A. et al. The MCL1 inhibitor S63845 is tolerable and effective in diverse cancer models. Nature 538, 477-482 (2016).

Götzke, H. et al. The ALFA-tag is a highly versatile tool for nanobody-based bioscience applications. Nat Commun 10, 4403 (2019).

STEFFEN, F., HANSJOERG, G., FELIPE, O. D. L., GUSTAV, S. P. E. & MARKEL, M. C. EPITOPE TAGS RECOGNIZED BY SPECIFIC BINDERS.

Zakeri, B. et al. Peptide tag forming a rapid covalent bond to a protein, through engineering a bacterial adhesin. Proc National Acad Sci 109, E690-E697 (2012).

Li, L., Fierer, J. O., Rapoport, T. A. & Howarth, M. Structural Analysis and Optimization of the Covalent Association between SpyCatcher and a Peptide Tag. J Mol Biol 426, 309-317 (2014).

Liu, Z. et al. Systematic comparison of 2A peptides for cloning multi-genes in a polycistronic vector. Sci Rep-uk 7, 2193 (2017).

Zhang, W. et al. Optogenetic control with a photocleavable protein, PhoCl. Nat Methods 14, 391-394 (2017).

Chung, H. K. et al. A compact synthetic pathway rewires cancer signaling to therapeutic effector release. Science eaat6982 (2019) doi: 10.1126/science.aat6982.

Drabkin, H. J. & RajBhandary, U. L. Initiation of Protein Synthesis in Mammalian Cells with Codons Other Than AUG and Amino Acids Other Than Methionine. Mol. Cell. Biol. 18, 5140-5147 (1998).

Stripecke, R., Oliveira, C. C., Mccarthy, J. E. G. & Hentze, M. W. Proteins Binding to 5′ Untranslated Region Sites: a General Mechanism for Translational Regulation of mRNAs in Human and Yeast Cells. Mol. Cell. Biol. 14, 5898-5909 (1994).

Buenrostro, J. D. et al. Quantitative analysis of RNA-protein interactions on a massively parallel array reveals biophysical and evolutionary landscapes. Nat Biotechnol 32, 562-568 (2014).

Johansson, H. E. et al. A thermodynamic analysis of the sequence-specific binding of RNA by bacteriophage MS2 coat protein. Proc National Acad Sci 95, 9244-9249 (1998).

Sharma, P. et al. 2A peptides provide distinct solutions to driving stop-carry on translational recoding. Nucleic Acids Res. 40, 3143-3151 (2012).

Feng, S. et al. Improved split fluorescent proteins for endogenous protein labeling. Nat Commun 8, 370 (2017).

Wood, T. I. et al. Defining the Role of Arginine 96 in Green Fluorescent Protein Fluorophore Biosynthesis †, ‡. Biochemistry 44, 16211-16220 (2005).

Götzke, H. et al. The ALFA-tag is a highly versatile tool for nanobody-based bioscience applications. Nat Commun 10, 4403 (2019).

STEFFEN, F., HANSJOERG, G., FELIPE, O. D. L., GUSTAV, S. P. E. & MARKEL, M. C. EPITOPE TAGS RECOGNIZED BY SPECIFIC BINDERS.

Tague, E. P., Dotson, H. L., Tunney, S. N., Sloas, D. C. & Ngo, J. T. Chemogenetic control of gene expression and cell signaling with antiviral drugs. Nat Methods 15, 519-522 (2018).

Jacobs, C. L., Badiee, R. K. & Lin, M. Z. StaPLs: versatile genetically encoded modules for engineering drug-inducible proteins. Nat. Methods 15, 523-526 (2018).

What is claimed herein is:

1. A fusion protein comprising:
(a) a first portion of a deaminase domain (DD) of an adenosine deaminase;
(b) a first member of a binding pair associated with the first portion of the DD;
(c) a second portion of the DD; and
(d) a second member of a binding pair associated with the second portion of the DD,
wherein the first member of the binding pair is capable of binding to the second member of the binding pair in the absence of an inducer, resulting in allosteric inhibition of the first and second portions of the DD, and
wherein the first member of the binding pair is not capable of binding to the second member of the binding pair in the presence of the inducer, resulting in activation of the first and second portions of the DD.

2. The fusion protein of claim 1, wherein in the allosteric inhibition of the first and second portions of the DD comprises deformation of the inositol hexaphosphate (IP6) binding pocket of first and second portions of the DD.

3. The fusion protein of claim 1, wherein in the activation of the first and second portions of the DD comprises deaminase activity.

4. The fusion protein of claim 1, further comprising an RNA-binding domain, wherein the RNA-binding domain (RBD) is capable of binding to a binding motif for the RBD on an RNA molecule.

5. The fusion protein of claim 4, wherein the RNA-binding domain is selected from the group consisting of MCP, PCP, λN, and HIV tat.

6. The fusion protein of claim 1, wherein the deaminase domain is capable of:
(a) deamination of an adenosine nucleotide into an inosine nucleotide in an RNA molecule;
(b) converting at least one stop codon into at least one non-stop codon;
(c) converting at least one start codon into at least one non-start codon;
(d) converting at least one non-start codon into at least one start codon;
and/or
(d) converting at least one sense codon encoding a first amino acid into at least one mutated sense codon encoding a second amino acid.

7. The fusion protein of claim 1, wherein the adenosine deaminase comprises Adenosine Deaminase Acting on RNA (ADAR), Adenosine Deaminase TRNA Specific (ADAT), or Adenosine Deaminase Domain Containing (ADAD).

8. The fusion protein of claim 7, wherein the ADAR is ADAR1, ADAR2, or ADAR3; the ADAT is ADAT1; or the ADAD is ADAD1 or ADAD2.

9. The fusion protein of claim 1, wherein in the presence of the inducer, the DD is constitutively active.

10. The fusion protein of claim 9, wherein the constitutively active deaminase domain comprises: an E1008Q mutation in ADAR1; an E488Q mutation in ADAR2; or an E527Q mutation in ADAR3.

11. The fusion protein of claim 1, wherein the DD comprises at least one mutation in the IP6 binding pocket that decreases background activity.

12. The fusion protein of claim 11, wherein the at least one mutation is in ADAR2 in an amino acid residue selected from the group consisting of: T375, R400, R522, Y658, K662, Y668, K672, V688, K690, F697, and L699.

13. The fusion protein of claim 4, comprising from N-terminus to C-terminus:
(a) the RNA-binding domain;
(b) the first portion of the deaminase domain;
(c) the first member of the binding pair;
(d) the second portion of the deaminase domain; and
(e) the second member of the binding pair; or
comprising from N-terminus to C-terminus
(f) the RNA-binding domain;
(g) the first member of the binding pair;
(h) the first portion of deaminase domain;
(i) the second portion of the deaminase domain; and
(j) the second member of the binding pair.

14. The fusion protein of claim 1, wherein the first and second portions of the deaminase domain are split at an RNA binding loop.

15. The fusion protein of claim 14, wherein the RNA binding loop comprises:
(a) residues G969 to K999 of ADAR1:
(SEQ ID NO: 134)
GALFDKSCSDRAMESTESRHYPVFENPKQGK
of ADAR1;
(b) residues A454 to Q479 of ADAR2:
(SEQ ID NO: 135)
ARIFSPHEPILEEPADRHPNRKARGQ;
(c) residues A493 to H518 of ADAR3:
(SEQ ID NO: 136)
ARLHSPYEITTDLHSSKHLVRKFRGH;
(d) residues A334 to K365 of ADAD1:
(SEQ ID NO: 137)
AQIKSQLRLNPHSISAFEANEELCLHVAVEGK;
(e) residues A347 to Q375 of ADAD2:
(SEQ ID NO: 138)
AARDIYLPPTSEGGLPHSPPMRLQAHVLGQ;
(f) residues K974 to S986 of ADAR1:
(SEQ ID NO: 139)
KSCSDRAMES;
(g) residues F457 to D469 of ADAR2:
(SEQ ID NO: 140)
FSPHEPILEEPAD;
(h) residues P498 to S508 of ADAR3:
(SEQ ID NO: 141)
PYEITTDLHSS;
(i) residues Q339 to P344 of ADADI:
(SEQ ID NO: 142)
QLRLNP;
or
(j) residues P352 to P360 of ADAD2:
(SEQ ID NO: 143)
PPTSEGGLP.

16. The fusion protein of claim 14, wherein the first and second portions of the deaminase domain are split between:
(a) residues S977 and D978 of ADAR1;
(b) residues T984 and E985 of ADAR1;
(c) residues A468 and D469 of ADAR2;
(d) residues S507 and S508 of ADAR3;
(e) residues L340 and R341 of ADAD1; or
(f) residues G357 and G358 of ADAD2.

17. The fusion protein of claim 1, wherein the first and second members of the binding pair are:
(a) Bad and Bcl-xL, and the inducer of the first and second binding pairs is A-1331852 or ABT-737;
(b) Bim and Bcl-xL, and the inducer of the first and second binding pairs is A-1331852;
(c) MS1 and MCL-1, and the inducer of the first and second binding pairs is S63845; or
(d) a repressible protease and a protease-binding peptide, and the inducer of the first and second binding pairs is an inhibitor of the repressible protease.

18. The fusion protein of claim 1, wherein:
(a) the first member of the binding pair comprises an antigen-binding domain,
(b) the second member of the binding pair comprises a first antigen, and
(c) the inducer of the first and second binding pairs comprises a second antigen;
wherein the antigen-binding domain is capable of binding to the second antigen with a similar or higher affinity than to the first antigen.

19. The fusion protein of claim 1, wherein the fusion protein further comprises a cleavable linker between the second portion of the deaminase domain the second member of the binding pair.

20. A fusion protein comprising:
(a) a first portion of a deaminase domain (DD) of an adenosine deaminase;
(b) a repressible protease associated with the first portion of the DD;
(c) a second portion of the DD; and
(d) a protease cleavage site associated with the first and second portions of the DD;
wherein the repressible protease is capable of binding to the protease cleavage site in the absence of an inhibitor for the repressible protease, resulting in cleavage of the protease cleavage site and inactivation of the first and second portions of the DD; and
wherein the repressible protease is not capable of binding to the protease cleavage site in the presence of the inhibitor for the repressible protease, resulting in activation of the first and second portions of the DD.

21. An RNA molecule comprising:
(I)
(a) a double-stranded region comprising:
(i) at least one target codon; and
(ii) an RNA binding motif capable of being bound by an RNA-binding domain; and
(b) at least one open reading frame, wherein the at least one open reading frame is operatively linked to the double-stranded region; or
(II)
(a) a first open reading frame;
(b) a double-stranded region comprising:
(i) at least one target codon; and
(ii) an RNA binding motif capable of being bound by an RNA-binding domain; and
(c) a second open reading frame, wherein the second open reading frame is operatively linked to the double-stranded region; or
(III)
(a) an open reading frame;
(b) a double-stranded region comprising:
(i) at least one target codon; and
(ii) an RNA binding motif capable of being bound by an RNA-binding domain; and
(c) a poly-A tail.

22. The RNA molecule of claim 21,
wherein the open reading frame encodes for a fusion protein or an effector protein;
wherein the first open reading frame encodes for a first polypeptide and the second open reading frame encodes for a second polypeptide;
wherein the first open reading frame encodes for a first portion of a polypeptide, and the second open reading frame encodes for a second portion of the polypeptide;
wherein the first or second open reading frame encodes for a fusion protein; or
wherein the first or second open reading frame encodes for at least one effector protein.

23. The RNA of claim 21, wherein the target codon is a stop codon, a start codon, a non-start codon, or a sense codon; and wherein the target codon comprises at least one adenosine nucleotide.

24. The RNA molecule of claim 21, wherein:
(a) the RNA-binding domain comprises MCP, and the RNA binding motif comprises MS2;
(b) the RNA-binding domain comprises PCP, and the RNA binding motif comprises PP7;
(c) the RNA-binding domain comprises λN, and the RNA binding motif comprises BoxB; or
(d) the RNA-binding domain comprises HIV Tat, and the RNA binding motif comprises TAR.

25. The RNA molecule of claim 21, wherein the double-stranded region of the RNA molecule comprises:
at least one hairpin.
one hairpin comprising the at least one target codon and the RNA binding motif; or
a first hairpin comprising the at least one target codon and a second hairpin comprising the RNA binding motif.

26. A system for modulating RNA translation comprising:
(a) a fusion protein comprising:
(i) an RNA-binding domain;
(ii) a first portion of a deaminase domain (DD) of an adenosine deaminase;
(iii) a first member of a binding pair associated with the first portion of the DD;
(iv) a second portion of the DD; and
(v) a second member of a binding pair associated with the second portion of the DD; and
(b) the RNA molecule of claim 21.

27. The system of claim 26, further comprising an inducer of the first and second binding pairs.

28. The system of claim 26, wherein the first and second members of the binding pair of the fusion protein are capable of binding to each other in the absence of an inducer of the first and second binding pairs and reduce or prevent:
(a) the formation of the inositol hexaphosphate (IP6) binding pocket of first and second portions of the deaminase domain;
(b) deaminase activity of the first and second portions of the deaminase domain;
(c) conversion by the deaminase domain of the at least one target codon of the RNA molecule into at least one inosine-comprising codon;
(d) conversion by the deaminase domain of the at least one stop codon of the RNA molecule into at least one non-stop codon;
(e) conversion by the deaminase domain of the at least one start codon of the RNA molecule into at least one non-start codon;
(f) conversion by the deaminase domain of the at least one non-start codon of the RNA molecule into at least one start codon; and/or
(g) conversion by the deaminase domain of the at least one sense codon of the RNA molecule encoding for a first amino acid into at least one mutated codon encoding for a second amino acid.

29. The system of claim 26, wherein the first and second members of the binding pair of the fusion protein are not capable of binding to each other in the presence of an inducer of the first and second binding pairs, allowing for:
(a) the formation of the inositol hexaphosphate (IP6) binding pocket of first and second portions of the deaminase domain;
(b) deaminase activity of the first and second portions of the deaminase domain;
(c) conversion by the deaminase domain of the at least one target codon of the RNA molecule into at least one inosine-comprising codon;
(d) conversion by the deaminase domain of the at least one stop codon of the RNA molecule into at least one non-stop codon;
(e) conversion by the deaminase domain of the at least one start codon of the RNA molecule into at least one non-start codon;
(f) conversion by the deaminase domain of the at least one non-start codon of the RNA molecule into at least one start codon; and/or
(g) conversion by the deaminase domain of the at least one sense codon of the RNA molecule encoding for a first amino acid into at least one mutated sense codon encoding for a second amino acid.

30. The system of claim 29, wherein conversion by the deaminase domain of at least one target codon in the RNA molecule into at least one inosine-comprising codon increases or decreases:
(a) translation of the reading frame of the RNA molecule;
(b) translation of the second reading frame of the RNA molecule;
(c) degradation of the RNA molecule; and/or
(d) translation of a variant polypeptide encoded by the RNA molecule.
